(19) United States
(12) Patent Application Publication
(10) Pub. No.: US 2022/0089592 A1
(43) Pub. Date: Mar. 24, 2022

(54) IMIDAZO[1,2-A]PYRIDINYL DERIVATIVES AS IRAK4 INHIBITORS

(71) Applicant: BIOGEN MA INC., Cambridge, MA (US)

(72) Inventors: Brian T. Hopkins, Newton, MA (US); Magnus Pfaffenbach, Chelsea, MA (US); Tricia May-dracka, Third Lake, IL (US); Ryan Evans, Somerville, MA (US); Fang Gao, Somerville, MA (US); Istvan Enyedy, Milton, MA (US); Zhili Xin, Lexington, MA (US); Philippe Bolduc, Weymouth, MA (US); Emily Anne Peterson, Belmont, MA (US)

(21) Appl. No.: 17/421,348

(22) PCT Filed: Jan. 17, 2020
(86) PCT No.: PCT/US2020/014126

§ 371 Date: Jul. 7, 2021

Related U.S. Application Data
(60) Provisional application No. 62/794,098, filed on Jan. 18, 2019.

(51) Int. Cl. C07D 471/04; C07D 519/00; C07D 487/04

(52) U.S. Cl. CPC C07D 471/04; C07D 487/04; C07D 519/00

(57) ABSTRACT

This invention relates to Imidazo[1,2-a]pyridinyl Derivatives of formula (I′), or pharmaceutically acceptable salts thereof, in which all of the variables are as defined in the specification, capable of modulating the activity of IRAK4. The invention further provides a method of manufacturing compounds of the invention, and methods for their therapeutic use. The invention further provides methods to their preparation, to their medical use, in particular to their use in the treatment and management of diseases or disorders including inflammatory disease, autoimmune disease, cancer, cardiovascular disease, a disease of the central nervous system, disease of the skin, an ophthalmic disease and condition, and a bone disease.

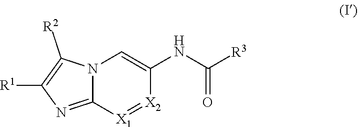

### RELATED APPLICATION

This application claims the benefit of the filing date under 35 U.S.C. § 119(e), of U.S. Provisional Patent Application No. 62/794,098, filed on Jan. 18, 2019, the entire content of which is hereby incorporated by reference.

### FIELD OF THE INVENTION

The present invention relates to imidazo[1,2-a]pyridinyl derivatives and pharmaceutically acceptable salts thereof, compositions of these compounds, either alone or in combination with at least one additional therapeutic agent, processes for their preparation, their use in the treatment of diseases, their use, either alone or in combination with at least one additional therapeutic agent and optionally in combination with a pharmaceutically acceptable carrier, for the manufacture of pharmaceutical preparations, use of the pharmaceutical preparations for the treatment of diseases, and a method of treatment of said diseases, comprising administering the Imidazo[1,2-a]pyridinyl derivatives to a warm-blooded animal, especially a human.

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is the protein kinase family.

Kinases catalyze the phosphorylation of proteins, lipids, sugars, nucleosides and other cellular metabolites and play key roles in all aspects of eukaryotic cell physiology. Especially, protein kinases and lipid kinases participate in the signaling events which control the activation, growth, differentiation and survival of cells in response to extracellular mediators or stimuli such as growth factors, cytokines or chemokines. In general, protein kinases are classified in two groups, those that preferentially phosphorylate tyrosine residues and those that preferentially phosphorylate serine and/or threonine residues.

Kinases are important therapeutic targets for the development of anti-inflammatory drugs (Cohen, 2009. Current Opinion in Cell Biology 21, 1-8), for example kinases that are involved in the orchestration of adaptive and innate immune responses. Kinase targets of particular interest are members of the IRAK family.

The interleukin-1 receptor-associated kinases (IRAKs) are critically involved in the regulation of intracellular signaling networks controlling inflammation (Ringwood and Li, 2008. Cytokine 42, 1-7). IRAKs are expressed in many cell types and can mediate signals from various cell receptors including toll-like receptors (TLRs). IRAK4 is thought to be the initial protein kinase activated downstream of the interleukin-1 (IL-1) receptor and all toll-like-receptors (TLRs) except TLR3, and initiates signaling in the innate immune system via the rapid activation of IRAK1 and slower activation of IRAK2. IRAK1 was first identified through biochemical purification of the IL-1 dependent kinase activity that co-immunoprecipitates with the IL-1 type 1 receptor (Cao et al., 1996. Science 271(5252): 1128-31). IRAK2 was identified by the search of the human expressed sequence tag (EST) database for sequences homologous to IRAK1 (Muzio et al., 1997. Science 278(5343): 1612-5). IRAK3 (also called IRAKM) was identified using a murine EST sequence encoding a polypeptide with significant homology to IRAK1 to screen a human phytohemagglutinin-activated peripheral blood leukocyte (PBL) cDNA library (Wesche et al., 1999. J. Biol. Chem. 274(27): 19403-10). IRAK4 was identified by database searching for IRAK-like sequences and PCR of a universal cDNA library (Li et al., 2002. Proc. Natl. Acad. Sci. USA 99(8):5567-5572). Many diseases are associated with abnormal cellular responses triggered by kinase-mediated events.

In particular, novel Imidazo[1,2-a]pyridinyl inhibitor compounds of formula (I) of the present invention possess a therapeutic role of inhibiting IRAK4 useful in the area of diseases and/or disorders that include, but are not limited to, cancers, allergic diseases, autoimmune diseases, inflammatory diseases and/or disorders and/or conditions associated with inflammation and pain, proliferative diseases, hematopoietic disorders, hematological malignancies, bone disorders, fibrosis diseases and/or disorders, metabolic disorders, muscle diseases and/or disorders, respiratory diseases, pulmonary disorders, genetic development diseases, neurological and neurodegenerative diseases and/or disorders, chronic inflammatory demyelinating neuropathies, cardiovascular, vascular or heart diseases, epilepsy, multiple sclerosis, Ischemic stroke, ophthalmic diseases, ocular diseases, asthma, Alzheimer's disease, Amyotrophic Lateral Sclerosis, Parkinson's disease, traumatic brain injury, Chronic Traumatic Encephalopathy and hormone-related diseases.

In view of the above, IRAK4 inhibitors of formula (I) are considered to be of value in the treatment and/or prevention for multiple therapeutic indications over a wide range of unmet needs.

### SUMMARY OF THE INVENTION

In a first aspect, the invention relates to a compound of formula (I):

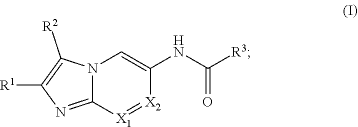

or a pharmaceutically acceptable salt thereof, wherein:

R¹ is selected from the group consisting of halogen, C₁₋₅ alkyl, C₃₋₆ cycloalkyl, —C₁₋₂ alkyl-C₃₋₆ cycloalkyl, C(O)NR⁴R⁵, C₁₋₄ alkyl-NR⁴R⁵, a fully saturated 4 to 7 membered heterocycle containing 1 to 2 heteroatoms independently selected from nitrogen, sulfur and oxygen, 5 to 6 membered heteroaryl, phenyl, —C₁₋₂ alkyl-C₄₋₇ heterocycle, wherein the C₄₋₇ heterocycle may be fully or partially saturated and contains 1 to 2 heteroatoms independently selected from nitrogen, sulfur and oxygen, —C₁₋₂ alkyl-C₅₋₆heteroaryl wherein the heteroaryl contains 1, 2 or 3 heteroatoms independently selected from nitrogen, sulfur and oxygen, —C₁₋₄ alkyl-O—C₁₋₂ alkyl, —C₁₋₂ alkyl-O—C₅₋₆ heteroaryl wherein the heteroaryl contains 1 or 2 heteroatoms independently selected from nitrogen, sulfur and oxygen, a fully saturated 5 to 10 membered bridged-carbocyclic ring, a 5 to 10 membered bridged-heterocyclic ring system having 1 to 2 heteroatoms independently selected from nitrogen and oxygen, a 5 to 10 membered fused heterobicyclic ring system having 1 to 2 heteroatoms independently selected from nitrogen and oxygen, and a 5 to 10 membered spiro heterobicyclic ring system having 1 to 2 heteroatoms independently selected from nitrogen and oxygen, wherein R¹ may be optionally substituted with 1, 2 or 3 substituents which are independently selected from halo, nitrile, oxo, —C(O)OH, halo-substitutedC₁₋₄ alkyl, hydroxy-substitutedC₁₋₄ alkyl, C₁₋₄ alkyl, C₄₋₇heterocycle containing 1 to 2 heteroatoms independently selected from nitrogen and oxygen, C₁₋₄ alkyl-O—C₁₋₂ alkyl, hydroxy, C(O)R⁶ and C₁₋₄ alkoxy;

R² is hydrogen, C₁₋₄ alkyl or halogen; or

R¹ and R² are taken together with their intervening atoms to form a 4 to 7 membered partially saturated carbocyclic ring or a partially saturated heterocyclic ring having one nitrogen said nitrogen may be optionally substituted with C₁₋₄ alkyl;

R⁴ and R⁵ are independently selected from hydrogen, hydroxy-substituted-C₁₋₄ alkyl and C₁₋₄ alkyl; or

R⁴ and R⁵ are taken together with the nitrogen to which they are connected form a 4 to 7 membered saturated heterocyclic ring, said ring optionally having an additional heteroatom selected from nitrogen and oxygen wherein said nitrogen may be optionally substituted with C₁₋₄ alkyl;

R⁶ is C₁₋₄ alkyl, halo-substituted-C₁₋₄ alkyl or O—C₁₋₄ alkyl;

R³ is selected from the group consisting of
i. a 5 or 6 membered heteroaryl having 1 to 2 heteroatoms independently selected from nitrogen, oxygen and sulfur, said heteroaryl is optionally substituted with 1 to 3 substituents independently selected from the group consisting of R⁷;
ii. phenyl optionally substituted with 1 to 3 substituents independently selected from the group consisting of R⁷;
iii. a 6 membered partially saturated heterocycle having 1 to 2 nitrogen atoms, said heterocycle may be optionally substituted with 1 to 3 substituents independently selected from the group consisting of R⁷; and
iv. an 8 to 10 membered fused bicyclic ring system optionally having 1, 2 or 3 heteroatoms independently selected from nitrogen and oxygen, said ring system is optionally substituted with 1 to 3 substituents independently selected from the group consisting of R⁷;

X₁ and X₂ are independently selected from N, CH and CR⁸; provided at least one of X₁ and X₂ is CR⁸;

R⁸ is selected from halogen, nitrile, NR⁴R⁵, —OR⁹ and a partially or fully saturated 4 to 7 membered heterocycle which contains 1 or 2 heteroatoms independently selected from nitrogen and oxygen, said C₁₋₄alkyl and said heterocycle is optionally substituted with 1 to 3 substituents selected from R^{4a};

R^{4a} for each occurrence, is independently selected from hydrogen, oxo, C₁₋₄ alkyl, halo-substitutedC₁₋₄ alkyl, C₁₋₄alkoxy, and C₃₋₆cycloalkyl; or two R^{4a} groups taken together with the carbon to which they are attached may combine to form a spiro 3-8 membered cycloalkyl;

R⁹ is hydrogen, a C₃₋₆cycloalkyl, a partially or fully saturated 4 to 7 membered heterocycle which contains 1 or 2 heteroatoms independently selected from nitrogen and oxygen, or a C₁₋₅alkyl, wherein said C₃₋₆cycloalkyl, said partially or fully saturated 4 to 7 membered heterocycle and said C₁₋₅alkyl represented by R⁹ are optionally substituted with 1 to 3 substituents independently selected from halogen, hydroxyl, nitrile, oxo, NR⁴R⁵, halo-substitutedC₁₋₄alkyl, C₁₋₄alkoxy, halo-substituted C₁₋₄alkoxy, C₃₋₆cycloalkyl, phenyl and a 4 to 7 membered partially or fully saturated heterocycle containing 1 or 2 heteroatoms selected from nitrogen and oxygen, wherein said C₃₋₆cycloalkyl, phenyl and 4 to 7 membered heterocycle may be optionally substituted with 1 to 3 R¹⁰;

R¹⁰ is independently selected from oxo, halo, halo-substitutedC₁₋₄ alkyl and C₁₋₄ alkyl;

R⁷ for each occurrence, is independently selected from C₁₋₄ alkyl, nitrile, oxo, halo, halo-substitutedC₁₋₄alkyl, —NR¹¹R¹², C₁₋₄ alkoxy, halo-substitutedC₁₋₄ alkoxy, a C₃₋₆cycloalkyl, a C₃₋₆cycloalkoxy, 4 to 7 membered partially or fully saturated heterocycle containing 1 or 2 heteroatoms selected from nitrogen and oxygen, and a 5 or 6 membered heteroaryl having 1 to 2 heteroatoms independently selected from nitrogen, oxygen and sulfur, said C₃₋₆cycloalkyl and heteroaryl may be optionally substituted with 1 to 2 substituents independently selected from the group consisting of C₁₋₄ alkyl, hydroxy and halogen and said C₁₋₄ alkyl and C₁₋₄alkoxy may be optionally substituted with C₁₋₄alkoxy; and

R¹¹ and R¹² are each independently selected from hydrogen, —C(O)C₁₋₄ alkyl and C₁₋₄ alkyl; or R¹¹ and R¹² may combine to form a 4 to 6 membered saturated ring optionally containing one additional heteroatom selected from nitrogen or oxygen wherein said additional nitrogen may be optionally substituted with C₁₋₄ alkyl,

provided when X₁ is CR⁸, X₂ is CH, and R⁸ is halogen, then R⁷ is not a 4 to 7 membered partially or fully saturated heterocycle containing 1 or 2 heteroatoms selected from nitrogen and oxygen; and provided that the compound is not:

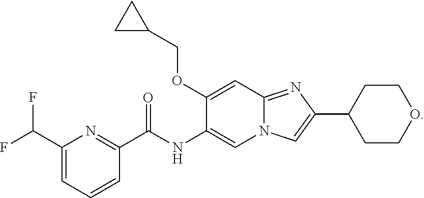 [SMILES: O=C(Nc1cn2cc(C3CCOCC3)nc2cc1OCC1CC1)c1cccc(C(F)F)n1]

In one embodiment, the present invention provides a compound of Formula (I): or a pharmaceutically acceptable salt thereof, wherein:

R¹ is selected from the group consisting of C₁₋₅ alkyl, C₃₋₆cycloalkyl, —C₁₋₂ alkyl-C₃₋₆cycloalkyl, C(O)NR⁴R⁵, C₁₋₄ alkyl-NR⁴R⁵, a fully saturated 5 to 10 membered bridged-carbocyclic ring, a fully saturated 4 to 7 membered heterocycle containing 1 to 2 heteroatoms independently selected from nitrogen, sulfur and oxygen, —C₁₋₂ alkyl-C₄₋₇ heterocycle, wherein the C₄₋₇ heterocycle may be fully or partially saturated and contains 1 to 2 heteroatoms independently selected from nitrogen, sulfur and oxygen, —C₁₋₂ alkyl-C₅₋₆ heteroaryl wherein the heteroaryl contains 1, 2 or 3 heteroatoms independently selected from nitrogen, sulfur and oxygen, —C₁₋₄ alkyl-O—C₁₋₂ alkyl, —C₁₋₂ alkyl-O—C₅₋₆ heteroaryl wherein the heteroaryl contains 1 or 2 heteroatoms independently selected from nitrogen, sulfur and oxygen, a 5 to 10 membered fused heterobicyclic ring system having 1 to 2 heteroatoms independently selected from nitrogen and oxygen and a 5 to 10 membered spiro heterobicyclic ring system having 1 to 2 heteroatoms independently selected from nitrogen and oxygen, wherein R¹ may be optionally substituted with 1, 2 or 3 substituents which are independently selected from halo, nitrile, oxo, halo-substitutedC₁₋₄ alkyl, hydroxy-substitutedC₁₋₄ alkyl, C₁₋₄ alkyl, C₄₋₇heterocycle containing 1 to 2 heteroatoms independently selected from nitrogen and oxygen, C₁₋₄ alkyl-O—C₁₋₂ alkyl, hydroxy, C(O)R⁶ and C₁₋₄ alkoxy;

R² is hydrogen, C₁₋₄ alkyl or halogen; or

R¹ and R² are taken together with their intervening atoms to form a 4 to 7 membered partially saturated carbocyclic ring or a partially saturated heterocyclic ring having one nitrogen said nitrogen may be optionally substituted with C₁₋₄ alkyl;

R⁴ and R⁵ are independently selected from hydrogen, hydroxy-substituted-C₁₋₄ alkyl and C₁₋₄ alkyl; or

R⁴ and R⁵ are taken together with the nitrogen to which they are connected form a 4 to 7 membered saturated heterocyclic ring, said ring optionally having an additional heteroatom selected from nitrogen and oxygen wherein said nitrogen may be optionally substituted with C₁₋₄ alkyl;

R⁶ is C₁₋₄ alkyl, halo-substitutedC₁₋₄ alkyl or O—C₁₋₄ alkyl;

R³ is selected from the group consisting of
i. a 5 or 6 membered heteroaryl having 1 to 2 heteroatoms independently selected from nitrogen, oxygen and sulfur, said heteroaryl is optionally substituted with 1 to 3 substituents independently selected from the group consisting of R⁷;
ii. Phenyl optionally substituted with 1 to 3 substituents independently selected from the group consisting of R⁷;
iii. a 6 membered partially saturated heterocycle having 1 to 2 nitrogen atoms, said heterocycle may be optionally substituted with 1 to 3 substituents independently selected from the group consisting of R⁷; and
iv. an 8 to 10 membered fused bicyclic ring system optionally having 1, 2 or 3 heteroatoms independently selected from nitrogen and oxygen, said ring system is optionally substituted with 1 to 3 substituents independently selected from the group consisting of R⁷;

X₁ and X₂ are independently selected from N, CH and CR⁸, wherein only one of X₁ or X₂ may be CR⁸;

R⁸ is selected from halogen, nitrile, NR⁴R⁵, —OR⁹ and a partially or fully or partially saturated 4 to 7 membered heterocycle which contains 1 or 2 heteroatoms independently selected from nitrogen and oxygen, said heterocycle is optionally substituted with 1 to 3 substituents selected from R^{4a};

R^{4a} for each occurrence, is independently selected from hydrogen, oxo, C₁₋₄ alkyl, halo-substitutedC₁₋₄ alkyl and C₃₋₆cycloalkyl; or two R^{4a} groups taken together with the carbon to which they are attached may combine to form a spiro 3-8 membered cycloalkyl;

R⁹ is hydrogen or an optionally substituted C₁₋₅alkyl having 1 to 3 substituents independently selected from halogen, hydroxyl, NR⁴R⁵, C₁₋₄alkoxy, C₃₋₆cycloalkyl, phenyl and a 4 to 7 membered partially or fully saturated heterocycle containing 1 or 2 heteroatoms selected from nitrogen and oxygen, wherein said C₃₋₆cycloalkyl, phenyl and 4 to 7 membered heterocycle may be optionally substituted with 1 to 3 R¹⁰;

R¹⁰ is independently selected from oxo, halo, halo-substitutedC₁₋₄ alkyl and C₁₋₄ alkyl;

R⁷ for each occurrence, is independently selected from C₁₋₄ alkyl, oxo, halo, halo-substitutedC₁₋₄alkyl, —NR¹¹R¹², C₁₋₄ alkoxy, halo-substitutedC₁₋₄ alkoxy, a C₃₋₆cycloalkyl and a 5 or 6 membered heteroaryl having 1 to 2 heteroatoms independently selected from nitrogen, oxygen and sulfur, said C₃₋₆cycloalkyl and heteroaryl may be optionally substituted with 1 to 2 substituents independently selected from the group consisting of C₁₋₄ alkyl, hydroxy and halogen; and

R¹¹ and R¹² are each independently selected from hydrogen, —C(O)C₁₋₄ alkyl and C₁₋₄ alkyl; or R¹¹ and R¹² may combine to form a 4 to 6 membered saturated ring optionally containing one additional heteroatom selected from nitrogen or oxygen wherein said additional nitrogen may be optionally substituted with C₁₋₄ alkyl;

or a pharmaceutically acceptable salt thereof.

Another aspect of the invention relates to pharmaceutical compositions comprising compounds of the invention or pharmaceutically acceptable salts thereof, and a pharmaceutical carrier. Such compositions can be administered in accordance with a method of the invention, typically as part of a therapeutic regimen for the treatment or prevention of conditions and disorders related to interleukin-1 receptor-associated kinases activity. In a particular aspect, the pharmaceutical compositions may additionally comprise further one or more therapeutically active ingredients suitable for the use in combination with the compounds of the invention. In a more particular aspect, the further therapeutically active ingredient is an agent for the treatment of autoimmune diseases, inflammatory diseases, bone diseases, metabolic diseases, neurological and neurodegenerative diseases, cancer, cardiovascular diseases, allergies, asthma, Alzheimer's disease, and hormone-related diseases. Accordingly, there remains a need to find protein kinase inhibitors useful as therapeutic agents.

Another aspect of the invention relates to the pharmaceutical combinations comprising compounds of the invention and other therapeutic agents for the use as a medicament in the treatment of patients having disorders related to interleukin-1 receptor-associated kinases activity. Such combinations can be administered in accordance with a method of the invention, typically as part of a therapeutic regiment for the treatment or prevention of autoimmune diseases, inflammatory diseases, bone diseases, metabolic diseases, neurological and neurodegenerative diseases, cancer, cardiovascular diseases, allergies, asthma, Alzheimer's disease, and hormone-related diseases. Accordingly, there remains a need to find protein kinase inhibitors useful as therapeutic agents.

### DETAILED DESCRIPTION OF THE INVENTION

The present invention provides compounds and pharmaceutical formulations thereof that may be useful in the treatment or prevention of conditions and/or disorders through mediation of IRAK4 function, such as neurological and neurodegenerative diseases, Alzheimer's disease, Ischemic stroke, Cerebral Ischemia, hypoxia, TBI (Traumatic Brain Injury), CTE (Chronic Traumatic Encephalopathy), epilepsy, multiple sclerosis (MS), Parkinson's disease (PD), and Amyotrophic Lateral Sclerosis (ALS).

In a first embodiment, the invention provides a compound of formula (I′):

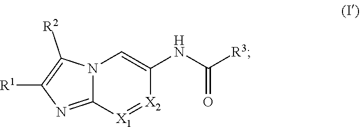

or a pharmaceutically acceptable salt thereof, wherein:

R¹ is selected from the group consisting of halogen, C₁₋₅ alkyl, C₃₋₆cycloalkyl, —C₁₋₂ alkyl-C₃₋₆cycloalkyl, C(O)NR⁴R⁵, C₁₋₄ alkyl-NR⁴R⁵, a fully or partially saturated 4 to 7 membered heterocycle containing 1 to 2 heteroatoms independently selected from nitrogen, sulfur and oxygen, 5 to 6 membered heteroaryl containing 1, 2 or 3 heteroatoms independently selected from nitrogen, sulfur and oxygen, phenyl, —C₁₋₂ alkyl-C₄₋₇ heterocycle, —C₁₋₂ alkyl-C₅₋₆ heteroaryl wherein —C₁₋₄ alkyl-O—C₁₋₂ alkyl, —C₁₋₂ alkyl-O—C₅₋₆heteroaryl, a fully saturated 5 to 10 membered bridged-carbocyclic ring, a 5 to 10 membered bridged-heterobicyclic ring system having 1 to 2 heteroatoms independently selected from nitrogen and oxygen, a 5 to 10 membered fused heterobicyclic ring system having 1 to 2 heteroatoms independently selected from nitrogen and oxygen and a 5 to 10 membered spiro heterobicyclic ring system having 1 to 2 heteroatoms independently selected from nitrogen and oxygen, wherein R¹ is optionally substituted with 1, 2 or 3 substituents independently selected from halo, nitrile, oxo, —C(O)OH, halo-substitutedC₁₋₄ alkyl, hydroxy-substitutedC₁₋₄ alkyl, C₁₋₄ alkyl, C₄₋₇ heterocycle containing 1 to 2 heteroatoms independently selected from nitrogen and oxygen, C₁₋₄ alkyl-O—C₁₋₂ alkyl, hydroxy, C(O)R⁶ and C₁₋₄ alkoxy, wherein the C₄₋₇ heterocycle described above is fully or partially saturated and contains 1 to 2 heteroatoms independently selected from nitrogen, sulfur and oxygen, and the C₅₋₆heteroaryl described above contains 1, 2 or 3 heteroatoms independently selected from nitrogen, sulfur and oxygen, and;

R² is hydrogen, C₁₋₄ alkyl or halogen; or

R¹ and R² are taken together with their intervening atoms to form a 4 to 7 membered partially saturated carbocyclic ring or a partially saturated heterocyclic ring having one nitrogen said nitrogen, wherein the 4 to 7 membered partially saturated carbocyclic ring and the partially saturated heterocyclic ring are optionally substituted with C₁₋₄ alkyl;

R⁴ and R⁵ are each independently selected from hydrogen, hydroxy-substituted-C₁₋₄ alkyl and C₁₋₄ alkyl; or

R⁴ and R⁵ are taken together with the nitrogen to which they are connected form a 4 to 7 membered saturated heterocyclic ring, said ring optionally having an additional heteroatom selected from nitrogen and oxygen wherein said nitrogen may be optionally substituted with C₁₋₄ alkyl;

R⁶ is C₁₋₄ alkyl, halo-substitutedC₁₋₄ alkyl or O—C₁₋₄ alkyl;

R³ is selected from the group consisting of
v. a 5 or 6 membered heteroaryl having 1 to 2 heteroatoms independently selected from nitrogen, oxygen and sulfur, said heteroaryl is optionally substituted with 1 to 3 substituents independently selected from the group consisting of R⁷;
vi. phenyl optionally substituted with 1 to 3 substituents independently selected from the group consisting of R⁷;
vii. a 6 membered partially saturated heterocycle having 1 to 2 nitrogen atoms, said heterocycle may be optionally substituted with 1 to 3 substituents independently selected from the group consisting of R⁷; and
viii. an 8 to 10 membered fused bicyclic ring system optionally having 1, 2 or 3 heteroatoms independently selected from nitrogen and oxygen, said ring system is optionally substituted with 1 to 3 substituents independently selected from the group consisting of R⁷;

X₁ and X₂ are independently selected from N, CH and CR⁸;

R⁸ is selected from C₁₋₄alkyl, halo-substituted C₁₋₄alkyl, hydroxy-substituted-C₁₋₄ alkyl, halogen, nitrile, NR⁴R⁵, —OR⁹ and a partially or fully saturated 4 to 7 membered heterocycle which contains 1 or 2 heteroatoms independently selected from nitrogen and oxygen, said C₁₋₄alkyl and said heterocycle is optionally substituted with 1 to 3 substituents selected from R^{4a};

R^{4a} for each occurrence, is independently selected from hydrogen, oxo, C₁₋₄ alkyl, halo-substitutedC₁₋₄ alkyl, C₁₋₄alkoxy, and C₃₋₆cycloalkyl; or two R^{4a} groups taken together with the carbon to which they are attached may combine to form a spiro 3-8 membered cycloalkyl;

R⁹ is hydrogen, a C₃₋₆cycloalkyl, a partially or fully saturated 4 to 7 membered heterocycle which contains 1 or 2 heteroatoms independently selected from nitrogen and oxygen, or a C₁₋₅alkyl, wherein said C₃₋₆cycloalkyl, said partially or fully saturated 4 to 7 membered heterocycle and said C₁₋₅alky represented by R⁹ are optionally substituted with 1 to 3 substituents independently selected from halogen, hydroxyl, nitrile, oxo, NR⁴R⁵, halo-substitutedC₁₋₄alkyl, C₁₋₄alkoxy, halo-substituted C₁₋₄alkoxy, C₃₋₆cycloalkyl, phenyl and a 4 to 7 membered partially or fully saturated heterocycle containing 1 or 2 heteroatoms selected from nitrogen and oxygen, wherein said C₃₋₆cycloalkyl, phenyl and 4 to 7 membered heterocycle may be optionally substituted with 1 to 3 R¹⁰;

R¹⁰ is independently selected from oxo, halo, halo-substitutedC₁₋₄ alkyl and C₁₋₄ alkyl;

R⁷ for each occurrence, is independently selected from C₁₋₄ alkyl, nitrile, oxo, halo, halo-substitutedC₁₋₄alkyl, —NR¹¹R¹², C₁₋₄ alkoxy, halo-substitutedC₁₋₄ alkoxy, a C₃₋₆cycloalkyl, a C₃₋₆cycloalkoxy, 4 to 7 membered partially or fully saturated heterocycle containing 1 or 2 heteroatoms selected from nitrogen and oxygen, and a 5 or 6 membered heteroaryl having 1 to 2 heteroatoms independently selected from nitrogen, oxygen and sulfur, said C₃₋₆cycloalkyl and heteroaryl may be optionally substituted with 1 to 2 substituents independently selected from the group consisting of C₁₋₄ alkyl, hydroxy and halogen and said C₁₋₄ alkyl and C₁₋₄alkoxy may be optionally substituted with C₁₋₄alkoxy; and

R¹¹ and R¹² are each independently selected from hydrogen, —C(O)C₁₋₄ alkyl and C₁₋₄ alkyl; or R¹¹ and R¹² may combine to form a 4 to 6 membered saturated ring optionally containing one additional heteroatom selected from nitrogen or oxygen wherein said additional nitrogen may be optionally substituted with C₁₋₄ alkyl.

In a specific embodiment, for compounds of formula (I′) or a pharmaceutically acceptable salt thereof described in the first embodiment, R⁸ is selected from halogen, nitrile, NR⁴R⁵, —OR⁹ and a partially or fully saturated 4 to 7 membered heterocycle which contains 1 or 2 heteroatoms independently selected from nitrogen and oxygen, said C₁₋₄alkyl and said heterocycle is optionally substituted with 1 to 3 substituents selected from R^{4a}; and the remaining variables are as defined in the first embodiment, provided when X₁ is CR⁸, X₂ is CH, and R⁸ is halogen, then R⁷ is not a 4 to 7 membered partially or fully saturated heterocycle containing 1 or 2 heteroatoms selected from nitrogen and oxygen; and provided that the compound is not:

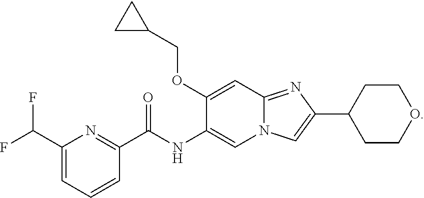 [SMILES: O=C(Nc1cn2cc(C3CCOCC3)nc2cc1OCC1CC1)c1cccc(C(F)F)n1]

In a second embodiment, the invention provides a compound of formula (I):

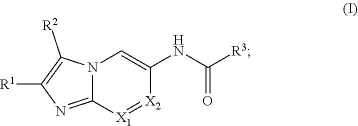

or a pharmaceutically acceptable salt thereof, wherein:

R¹ is selected from the group consisting of C₁₋₅ alkyl, C₃₋₆cycloalkyl, —C₁₋₂ alkyl-C₃₋₆cycloalkyl, C(O)NR⁴R⁵, C₁₋₄alkyl-NR⁴R⁵, a fully saturated 4 to 7 membered heterocycle containing 1 to 2 heteroatoms independently selected from nitrogen, sulfur and oxygen, —C₁₋₂ alkyl-C₄₋₇ heterocycle, wherein the C₄₋₇heterocycle may be fully or partially saturated and contains 1 to 2 heteroatoms independently selected from nitrogen, sulfur and oxygen, —C₁₋₂ alkyl-C₅₋₆heteroaryl wherein the heteroaryl contains 1, 2 or 3 heteroatoms independently selected from nitrogen, sulfur and oxygen, —C₁₋₄ alkyl-O—C₁₋₂ alkyl, —C₁₋₂ alkyl-O—C₅₋₆heteroaryl wherein the heteroaryl contains 1 or 2 heteroatoms independently selected from nitrogen, sulfur and oxygen, a fully saturated 5 to 10 membered bridged-carbocyclic ring, a 5 to 10 membered fused heterobicyclic ring system having 1 to 2 heteroatoms independently selected from nitrogen and oxygen and a 5 to 10 membered spiro heterobicyclic ring system having 1 to 2 heteroatoms independently selected from nitrogen and oxygen, wherein R¹ may be optionally substituted with 1, 2 or 3 substituents which are independently selected from halo, nitrile, oxo, halo-substitutedC₁₋₄ alkyl, hydroxy-substitutedC₁₋₄ alkyl, C₁₋₄ alkyl, C₄₋₇heterocycle containing 1 to 2 heteroatoms independently selected from nitrogen and oxygen, C₁₋₄ alkyl-O—C₁₋₂ alkyl, hydroxy, C(O)R⁶ and C₁₋₄ alkoxy;

R² is hydrogen, C₁₋₄ alkyl or halogen; or

R¹ and R² are taken together with their intervening atoms to form a 4 to 7 membered partially saturated carbocyclic ring or a partially saturated heterocyclic ring having one nitrogen said nitrogen may be optionally substituted with C₁₋₄ alkyl;

R⁴ and R⁵ are independently selected from hydrogen, hydroxy-substituted-C₁₋₄ alkyl and C₁₋₄ alkyl; or

R⁴ and R⁵ are taken together with the nitrogen to which they are connected form a 4 to 7 membered saturated heterocyclic ring, said ring optionally having an additional heteroatom selected from nitrogen and oxygen wherein said nitrogen may be optionally substituted with C₁₋₄ alkyl;

R⁶ is C₁₋₄ alkyl, halo-substitutedC₁₋₄ alkyl or O—C₁₋₄ alkyl;

R³ is selected from the group consisting of
i. a 5 or 6 membered heteroaryl having 1 to 2 heteroatoms independently selected from nitrogen, oxygen and sulfur, said heteroaryl is optionally substituted with 1 to 3 substituents independently selected from the group consisting of R⁷;
ii. phenyl optionally substituted with 1 to 3 substituents independently selected from the group consisting of R⁷;
iii. a 6 membered partially saturated heterocycle having 1 to 2 nitrogen atoms, said heterocycle may be optionally substituted with 1 to 3 substituents independently selected from the group consisting of R⁷; and
iv. an 8 to 10 membered fused bicyclic ring system optionally having 1, 2 or 3 heteroatoms independently selected from nitrogen and oxygen, said ring system is optionally substituted with 1 to 3 substituents independently selected from the group consisting of R⁷;

X₁ and X₂ are independently selected from N, CH and CR⁸, wherein only one of X₁ or X₂ may be CR⁸;

R⁸ is selected from halogen, nitrile, NR⁴R⁵, —OR⁹ and a partially or fully or partially saturated 4 to 7 membered heterocycle which contains 1 or 2 heteroatoms independently selected from nitrogen and oxygen, said heterocycle is optionally substituted with 1 to 3 substituents selected from R^{4a}; R^{4a} for each occurrence, is independently selected from hydrogen, oxo, C₁₋₄ alkyl, halo-substitutedC₁₋₄ alkyl and C₃₋₆cycloalkyl; or two R^{4a} groups taken together with the carbon to which they are attached may combine to form a spiro 3-8 membered cycloalkyl;

R⁹ is hydrogen or an optionally substituted C₁₋₅alkyl having 1 to 3 substituents independently selected from halogen, hydroxyl, NR⁴R⁵, C₁₋₄alkoxy, C₃₋₆cycloalkyl, phenyl and a 4 to 7 membered partially or fully saturated heterocycle containing 1 or 2 heteroatoms selected from nitrogen and oxygen, wherein said C₃₋₆cycloalkyl, phenyl and 4 to 7 membered heterocycle may be optionally substituted with 1 to 3 R¹⁰;

R¹⁰ is independently selected from oxo, halo, halo-substitutedC₁₋₄ alkyl and C₁₋₄ alkyl;

R⁷ for each occurrence, is independently selected from C₁₋₄ alkyl, oxo, halo, halo-substitutedC₁₋₄alkyl, —NR¹¹R¹², C₁₋₄ alkoxy, halo-substitutedC₁₋₄ alkoxy, a C₃₋₆cycloalkyl and a 5 or 6 membered heteroaryl having 1 to 2 heteroatoms independently selected from nitrogen, oxygen and sulfur, said C₃₋₆cycloalkyl and heteroaryl may be optionally substituted with 1 to 2 substituents independently selected from the group consisting of C₁₋₄ alkyl, hydroxy and halogen; and

R¹¹ and R¹² are each independently selected from hydrogen, —C(O)C₁₋₄ alkyl and C₁₋₄ alkyl; or R¹¹ and R¹² may combine to form a 4 to 6 membered saturated ring optionally containing one additional heteroatom selected from nitrogen or oxygen wherein said additional nitrogen may be optionally substituted with C₁₋₄ alkyl;

or a pharmaceutically acceptable salt thereof.

In a specific embodiment, for compounds of formula (I′) or (I) or a pharmaceutically acceptable salt thereof described in the first or second embodiment, when X₁ and X₂ are CH and R³ is phenyl, then R⁷ is not a 5 or 6 membered heteroaryl and provided that the compound is not

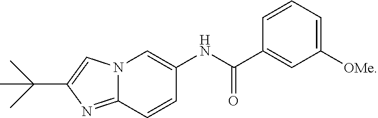 [SMILES: COc1cccc(C(=O)Nc2ccc3nc(C(C)(C)C)cn3c2)c1]

In another specific embodiment, for compounds of formula (I′) or (I) or a pharmaceutically acceptable salt thereof described in the first or second embodiment, the compound is not:

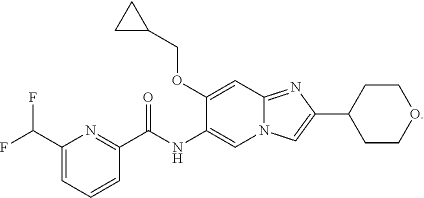 [SMILES: O=C(Nc1cn2cc(C3CCOCC3)nc2cc1OCC1CC1)c1cccc(C(F)F)n1]

In another specific embodiment, for compounds of formula (I′) or (I), or a pharmaceutically acceptable salt thereof described in the first or second embodiment, at least one of X₁ and X₂ are CR⁸.

In a third embodiment, the invention provides a compound of formula (Ia):

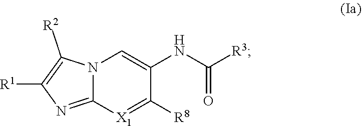

or a pharmaceutically acceptable salt thereof, wherein X₁ is N or CH; and the remaining variables are as defined in the first or second embodiment or any specific embodiments described therein.

In a fourth embodiment, the invention provides a compound of formula (Ib):

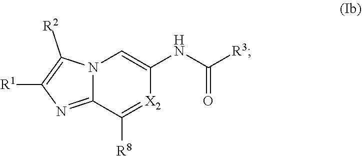

or a pharmaceutically acceptable salt thereof, wherein X₂ is N or CH; and the remaining variables are as defined in the first or second embodiment or any specific embodiments described therein.

A fifth embodiment of the invention provides a compound or a pharmaceutically acceptable salt thereof according to any of the preceding embodiments wherein:

R¹ is selected from the group consisting of C₁₋₅ alkyl, C₃₋₆cycloalkyl, —C₁₋₂ alkyl-C₃₋₆ cycloalkyl, C(O)NR⁴R⁵, C₁₋₄ alkyl-NR⁴R⁵, a fully saturated 4 to 7 membered heterocycle containing 1 to 2 heteroatoms independently selected from nitrogen, sulfur and oxygen, —C₁₋₂ alkyl-C₄₋₇ heterocycle, wherein the C₄₋₇heterocycle may be fully or partially saturated and contains 1 to 2 heteroatoms independently selected from nitrogen, sulfur and oxygen, —C₁₋₂ alkyl-C₅₋₆heteroaryl wherein the heteroaryl contains 1, 2 or 3 heteroatoms independently selected from nitrogen, sulfur and oxygen, —C₁₋₄ alkyl-O—C₁₋₂ alkyl, —C₁₋₂ alkyl-O—C₅₋₆heteroaryl wherein the heteroaryl contains 1 or 2 heteroatoms independently selected from nitrogen, sulfur and oxygen, a 5 to 10 membered fused heterobicyclic ring system having 1 to 2 heteroatoms independently selected from nitrogen and oxygen and a 5 to 10 membered spiro heterobicyclic ring system having 1 to 2 heteroatoms independently selected from nitrogen and oxygen, wherein R¹ may be optionally substituted with 1, 2 or 3 substituents which are independently selected from halo, nitrile, oxo, halo-substitutedC₁₋₄ alkyl, hydroxy-substitutedC₁₋₄ alkyl, C₁₋₄ alkyl, C₄₋₇heterocycle containing 1 to 2 heteroatoms independently selected from nitrogen and oxygen, C₁₋₄ alkyl-O—C₁₋₂ alkyl, hydroxy, C(O)R⁶ and C₁₋₄ alkoxy;

R² is hydrogen, C₁₋₄ alkyl or halogen; and the remaining variables are as defined in the first, second, third or fourth embodiment or any specific embodiments described therein.

In a sixth embodiment, the invention provides a compound of formula (IIa):

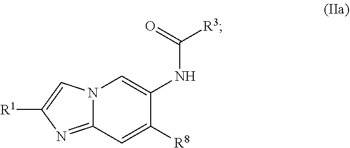

or a pharmaceutically acceptable salt thereof, wherein the variables are as defined in the first, second or fifth embodiment or any specific embodiments described therein.

In a seventh embodiment, the invention provides a compound of formula (IIb):

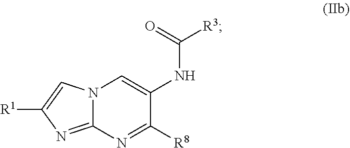

or a pharmaceutically acceptable salt thereof, wherein the variables are as defined in the first, second or fifth embodiment or any specific embodiments described therein.

In a eighth embodiment, the invention provides a compound of formula (IIc):

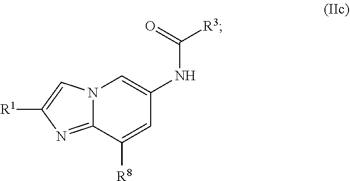

or a pharmaceutically acceptable salt thereof, wherein the variables are as defined in the first, second or fifth embodiment or any specific embodiments described therein.

In a ninth embodiment, the invention provides a compound of formula (IId):

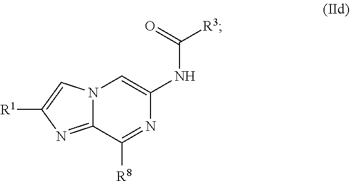

or a pharmaceutically acceptable salt thereof, wherein the variables are as defined in the first, second or fifth embodiment or any specific embodiments described therein.

In a tenth embodiment, the invention provides a compound of formula (IIe):

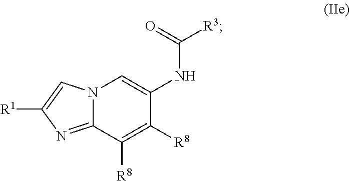

or a pharmaceutically acceptable salt thereof, wherein the variables are as defined in the first or fifth embodiment or any specific embodiments described therein.

In an eleventh embodiment, the invention provides a compound of formula (IIIa):

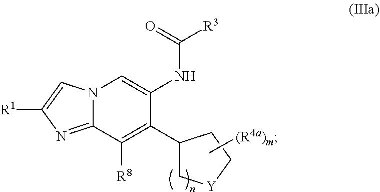

or a pharmaceutically acceptable salt thereof, wherein:

Y is nitrogen, carbon or oxygen;

n is 1 or 2; and

m is 0, 1, 2 or 3; and the remaining variables are as define in the first, second or fifth embodiment or any specific embodiments described therein.

In a twelfth embodiment, the invention provides a compound of formula (IIIb):

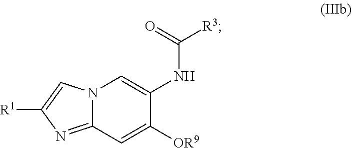

or a pharmaceutically acceptable salt thereof, wherein the variables are as define in the first, second or fifth embodiment or any specific embodiments described therein.

In a thirteenth embodiment, the invention provides a compound of formula (IIIc):

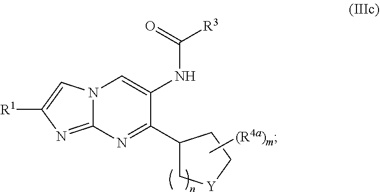

or a pharmaceutically acceptable salt thereof, wherein:

Y is nitrogen, carbon or oxygen;

n is 1 or 2; and

m is 0, 1, 2 or 3; and the remaining variables are as define in the first, second or fifth embodiment or any specific embodiments described therein.

In a fourteenth embodiment, the invention provides a compound of formula (IIId):

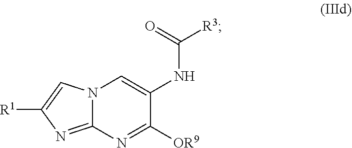

or a pharmaceutically acceptable salt thereof, wherein the variables are as define in the first, second or fifth embodiment or any specific embodiments described therein.

In a fifteenth embodiment, the invention provides a compound of formula (IIIe):

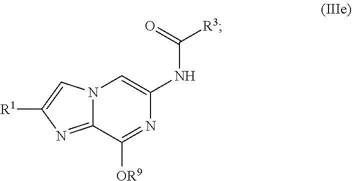

or a pharmaceutically acceptable salt thereof, wherein the variables are as define in the first, second or fifth embodiment or any specific embodiments described therein.

In a sixteenth embodiment, the invention provides a compound of formula (IIIf):

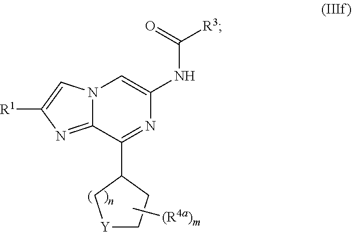

or a pharmaceutically acceptable salt thereof, wherein:

Y is nitrogen, carbon or oxygen;

n is 1 or 2; and

m is 0, 1, 2 or 3; and

the remaining variables are as define in the first, second or fifth embodiment or any specific embodiments described therein.

In a seventeenth embodiment, the invention provides a compound of formula (IIIg):

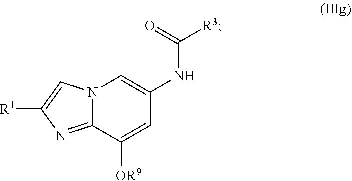

or a pharmaceutically acceptable salt thereof, wherein the variables are as define in the first, second or fifth embodiment or any specific embodiments described therein.

In an eighteenth embodiment, the invention provides a compound of formula (IIIh):

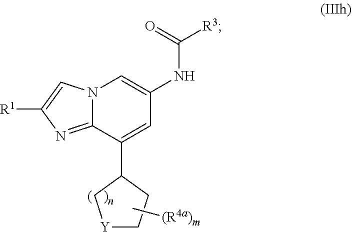

or a pharmaceutically acceptable salt thereof, wherein:

Y is nitrogen, carbon or oxygen;

n is 1 or 2; and

m is 0, 1, 2 or 3; and the remaining variables are as define in the first, second or fifth embodiment or any specific embodiments described therein.

In a nineteenth embodiment, the invention provides a compound of formula (IIIi) or (IIIj)

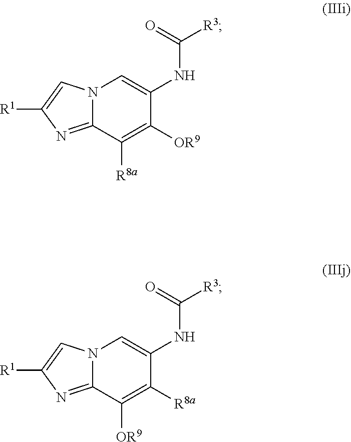

or a pharmaceutically acceptable salt thereof, wherein R^{8a} is C₁₋₄alkyl, halo-substituted C₁₋₄alkyl, or halogen; and the remaining variables are as define in the first, second or fifth embodiment or any specific embodiments described therein.

In a twentieth embodiment, for compounds or pharmaceutically acceptable salts thereof described in any one of the preceding embodiments (e.g., compounds of formula (I′), (I), (Ia), (Ib), (IIa), (IIb), (IIc), (IId), (IIe), (IIIb), (IIId), (IIIe), (IIIg), (IIIh), (IIIi), or (IIIj) or pharmaceutically acceptable salts thereof), R⁹ is hydrogen or an optionally substituted C₁₋₅alkyl having 1 to 3 substituents independently selected from halogen, hydroxyl, NR⁴R⁵, C₁₋₄alkoxy, phenyl and a 4 to 7 membered partially or fully saturated heterocycle containing 1 or 2 heteroatoms selected from nitrogen and oxygen, wherein said phenyl and 4 to 7 membered heterocycle may be optionally substituted with 1 to 3 R¹⁰; R¹⁰ is independently selected from oxo, halo, halo-substitutedC₁₋₄ alkyl and C₁₋₄ alkyl; and the remaining variables are as defined in any of the preceding embodiments.

In a twenty-first embodiment, for compounds or pharmaceutically acceptable salts thereof described in any one of the preceding embodiments (e.g., compounds of formula (I′), (I), (Ia), (Ib), (IIa), (IIb), (IIc), (IId), (IIe), (IIIb), (IIId), (IIIe), (Thug), (IIIh), (IIIi), or (IIIj) or pharmaceutically acceptable salts thereof), R⁹ is C₁₋₅alkyl; and the remaining variables are as defined in any of the preceding embodiments.

In a twenty-second embodiment, the invention provides a compound or a pharmaceutically acceptable salt thereof according to any of the preceding embodiments, wherein:

R³ is selected from the group consisting of
i. a 5 to 6 membered heteroaryl selected from pyridyl, oxazolyl, pyrazinyl, oxadazoyl, thiazolyl, pyrazolyl and imidazolyl, said heteroaryl is optionally substituted with 1 to 2 substituents independently selected from the group consisting of R⁷;
ii. phenyl optionally substituted with 1 to 2 substituents independently selected from the group consisting of R⁷;
iii. pyridinyl-2(1H)-one optionally substituted with 1 to 2 substituents independently selected from the group consisting of R⁷; and
iv. an 8 to 10 membered fused bicyclic ring system selected from the group consisting of 1,3-dihydroisobenzofuran, 4-methyl-3,4-dihydro-2H-benzo[b][1,4]oxazine, 5,6-dihydro-4H-pyrrolo[1,2-b]pyrazole, 6,7-dihydro-5H-cyclopenta[b]pyridine, pyrazolo[1,5-a]pyrimidine, indolin-2-one, 2,3-dihydrobenzofuran, 1-methyl-2-oxo-1,2,3,4-tetrahydroquinoline, 3,4-dihydroquinolin-2(1H)-one, and isochromane, wherein said fused bicyclic ring system is optionally substituted with 1 to 2 substituents independently selected from the group consisting of R⁷.

In a twenty-third embodiment, the invention provides a compound or a pharmaceutically acceptable salt thereof according to any of the preceding embodiments, wherein: R³ is selected from pyridyl, oxazolyl, pyrazinyl, oxadazoyl, thiazolyl, pyrazolyl, imidazolyl, said R³ is optionally substituted with 1 to 2 substituents independently selected from the group consisting of halo, halo-substitutedC₁₋₄ alkyl, —NR¹¹R¹², and C₁₋₄ alkyl.

In a twenty-fourth embodiment, the invention provides a compound or a pharmaceutically acceptable salt thereof according to the first through twenty-second embodiments wherein: R³ is pyridinyl-2(1H)-one optionally substituted with 1 to 2 substituents independently selected from the group consisting of halo, halo-substitutedC₁₋₄ alkyl, —NR¹¹R¹², and C₁₋₄ alkyl.

In a twenty-fifth embodiment, the invention provides a compound or a pharmaceutically acceptable salt thereof according to the first through twenty-second embodiments, wherein: R³ is phenyl, said phenyl is optionally substituted with 1 to 2 substituents independently selected from the group consisting of halo, halo-substitutedC₁₋₄ alkyl, —NR¹¹R¹², and C₁₋₄ alkyl.

In a twenty-sixth embodiment, the invention provides a compound or a pharmaceutically acceptable salt thereof according to the first through twenty-second embodiments, wherein:

R³ is selected from the group consisting of 1,3-dihydroisobenzofuran, 4-methyl-3,4-dihydro-2H-benzo[b][1,4]oxazine, 5,6-dihydro-4H-pyrrolo[1,2-b]pyrazole, 6,7-dihydro-5H-cyclopenta[b]pyridine, indolin-2-one, 2,3-dihydrobenzofuran, pyrazolo[1,5-a]pyrimidine, 1-methyl-2-oxo-1,2,3,4-tetrahydroquinoline, 3,4-dihydroquinolin-2(1H)-one, and isochromane, wherein said R³ is optionally substituted with 1 to 2 substituents independently selected from the group consisting halo, halo-substitutedC₁₋₄ alkyl, —NR¹¹R¹², and C₁₋₄ alkyl.

In a twenty-seventh embodiment, the invention provides a compound or a pharmaceutically acceptable salt thereof according to the first through twenty-second embodiments, wherein:

R³ is an 8 to 10 membered fused bicyclic heteroaryl or an 8 to 10 membered fused heterobicyclic ring, each of which is optionally substituted with 1 or 2 substituents independently selected from the group consisting halo, halo-substitutedC₁₋₄ alkyl, —NR¹¹R¹², C₁₋₄alkoxy and C₁₋₄ alkyl; and the remaining variables are as described in the first through twenty-second embodiments.

In a twenty-eighth embodiment, the invention provides a compound or a pharmaceutically acceptable salt thereof according to the first through twenty-second embodiments, wherein:

R³ is selected from 2,3-dihydrofuro[2,3-b]pyridine, 5,6,7,8-tetrahydroindolizine, 2,3-dihydrothieno[3,4-b][1,4]dioxine, 6,7-dihydro-5H-cyclopenta[b]pyridine, 2,3-dihydrobenzofuran, 2,3-dihydro-1H-pyrrolizine, pyrazolo[1,5-a]pyridine, pyrazolo[1,5-a]pyrimidine, and [1,2,4]triazolo[1,5-a]pyridine, each of which is optionally substituted with 1 or 2 substituents independently selected from the group consisting of halo, halo-substitutedC₁₋₄ alkyl, —NR¹¹R¹², C₁₋₄alkoxy and C₁₋₄ alkyl; and the remaining variables are as defined in the first through twenty-second embodiments.

In a twenty-ninth embodiment, the invention provides a compound or a pharmaceutically acceptable salt thereof according to the first through twenty-second embodiments, wherein: R³ is pyridyl optionally substituted with 1 or 2 substituents independently selected from R⁷,

R⁷ is nitrile, halo, C₁₋₄alkyl, halo-substitutedC₁₋₄alkyl, C₁₋₄alkoxy, halo-substitutedC₁₋₄alkoxy, C₃₋₆cycloalkyl, C₃₋₆cycloalkoxy, —NR¹¹R¹², and 4 to 7 membered fully saturated heterocycle containing 1 or 2 heteroatoms selected fro nitrogen and oxygen, wherein the C₁₋₄alkyl and C₁₋₄alkoxy is optionally substituted with C₁₋₄alkoxy;

R¹¹ and R¹² are each independently hydrogen or C₁₋₄alkyl; and the remaining variables are as defined in the first through twenty-second embodiments.

In a specific embodiment, for compounds or pharmaceutically acceptable salts thereof described in the twenty-ninth embodiment, R³ is 2-pyridyl or 3-pyridyl, each of which is optionally substituted with 1 or 2 substituents independently selected from R⁷; and the remaining variables are as described in the twenty-ninth embodiment.

In a thirtieth embodiment, for compounds or pharmaceutically acceptable salts thereof described in the twenty-ninth embodiment, R⁷, for each occurrence, is independently tetrahydrofuran, F, —CN, —CH₃, —CH(CH₃)₂, —CH₂CH₃, —CHF—CH₂F, —CHF₂, —CF₂CH₃, —CF₃, —OCH₃, —OCH(CH₃)₂, —OCHF₂, —OCH₂CF₃, —OCH₂CHF₂, —OCH₂CH₂OCH₃, —CH₂OCH₃, —O— cyclopropyl, —O-cyclobutyl, cyclopropyl, or —N(CH₃)₂; and the remaining variables are as described in the twenty-ninth embodiment.

A thirty-first embodiment of the invention provides a compound of formula (IV):

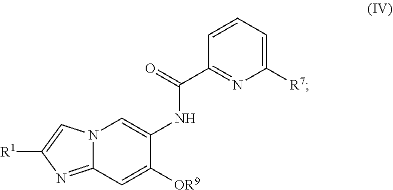

or a pharmaceutically acceptable salt thereof, wherein:

R⁹ is an optionally substituted C₁₋₅alkyl having 1 to 3 substituents independently selected from halogen, hydroxyl, NR⁴R⁵, C₁₋₄alkoxy, C₃₋₆cycloalkyl, phenyl and a 4 to 7 membered partially or fully saturated heterocycle containing 1 or 2 heteroatoms selected from nitrogen and oxygen, wherein said C₃₋₆cycloalkyl, phenyl and 4 to 7 membered heterocycle may be optionally substituted with 1 to 3 R¹⁰; and the remaining variables are as defined in the first or second embodiment.

A third-second embodiment of the invention provides a compound or a pharmaceutically acceptable salt thereof according to any one of the preceding embodiments, wherein:

R¹ is C₃₋₆cycloalkyl optionally substituted with 1 or 2 substituents independently selected from the group consisting of C₁₋₄alkyl, halogen, halo-substitutedC₁₋₄ alkyl, hydroxyl and C₁₋₄alkoxy, or R¹ is a C_{1I}_alkyl which is optionally substituted with 1 or 3 substituents independently selected from the group consisting of halogen, halo-substitutedC₁₋₄ alkyl, hydroxy-substitutedC₁₋₄ alkyl, hydroxyl, C₁₋₄alkoxy and C₃₋₆cycloalkyl, wherein said C₃₋₆cycloalkyl is optionally substituted with 1 or 2 substituents independently selected from the group consisting of halogen, halo-substitutedC₁₋₄ alkyl, hydroxyl and C₁₋₄alkoxy; and the remaining variables are as defined in any one of the preceding embodiments.

A thirty-third embodiment of the invention provides a compound or a pharmaceutically acceptable salt thereof according to any one of the preceding embodiments, wherein: R¹ is an C₃₋₆cycloalkyl optionally substituted with 1 or 2 substituents independently selected from the group consisting of C₁₋₄alkyl, halogen, halo-substitutedC₁₋₄ alkyl, hydroxyl and C₁₋₄alkoxy; and the remaining variables are as defined in any one of the preceding embodiments.

A thirty-fourth embodiment of the invention provides a compound or a pharmaceutically acceptable salt thereof according to any one of embodiments one to thirty-first, wherein: R¹ is a C₁₋₅ alkyl which is optionally substituted with 1 or 3 substituents independently selected from the group consisting of halogen, halo-substitutedC₁₋₄ alkyl, hydroxyl, C₁₋₄alkoxy and C₃₋₆cycloalkyl, wherein said C₃₋₆cycloalkyl is optionally substituted with 1 or 2 substituents independently selected from the group consisting of halogen, halo-substitutedC₁₋₄ alkyl, hydroxyl and C₁₋₄alkoxy; and the remaining variables are as defined in any one of embodiments one to thirty-first.

A thirty-fifth embodiment of the invention provides a compound or a pharmaceutically acceptable salt thereof according to any one of embodiments one to thirty, wherein:

R¹ is an C₃₋₆cycloalkyl optionally substituted with 1 or 2 substituents independently selected from the group consisting of C₁₋₄alkyl, halogen, halo-substitutedC₁₋₄ alkyl, hydroxyl and C₁₋₄alkoxy; and

R³ is optionally substituted with 1 or 2 substituents independently selected from and C₁₋₄ alkyl and halo-substitutedC₁₋₄ alkyl; and the remaining variables are as defined in any one of embodiments one to thirty.

A thirty-sixth embodiment of the invention provides a compound or a pharmaceutically acceptable salt thereof according to any one of embodiments one to thirty-first, wherein R¹ is a 5 to 10 membered bridged-heterocyclic ring containing 1 to 3 heteroatoms independently selected from nitrogen and oxygen; wherein the 5 to 10 membered bridged-heterocyclic ring is optionally substituted with 1 or 3 substituents independently selected from C₁₋₄alkyl, halogen, halo-substitutedC₁₋₄ alkyl, hydroxyl and C₁₋₄alkoxy; and the remaining variables are as defined in the first to thirty-first embodiments. In a specific embodiment, R¹ is a 6 to 8 membered bridged-heterocyclic ring containing 1 or 2 heteroatoms independently selected from nitrogen and oxygen, wherein the 6 to 8 membered bridged-heterocyclic ring is optionally substituted with 1 or 2 substituents independently selected from C₁₋₄alkyl, halogen, halo-substitutedC₁₋₄ alkyl, hydroxyl and C₁₋₄alkoxy.

A thirty-seventh embodiment provides a compound or a pharmaceutically acceptable salt thereof according to any one of embodiments one to thirty-first, wherein R¹ is selected from 2-oxabicyclo[2.1.1]hexan-4-yl 8-oxabicyclo[3.2.1]octan-3-yl, 2-oxabicyclo[2.2.1]heptan-4-yl, 2-oxabicyclo[2.2.2]octan-4-yl, 2-oxabicyclo[3.1.1]heptan-5-yl, each of which is optionally substituted with 1 to 3 substituents independently selected from C₁₋₄alkyl, halogen, halo-substitutedC₁₋₄ alkyl hydroxyl and C₁₋₄alkoxy. In a specific embodiment R is selected from:

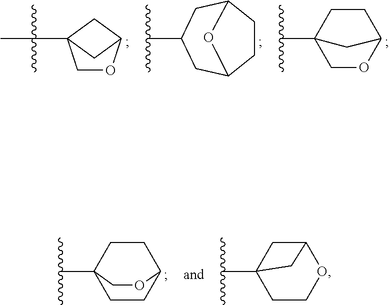

each of which is optionally substituted with 1 to 3 substituents independently selected from C₁₋₄alkyl, halogen, halo-substitutedC₁₋₄ alkyl, hydroxyl and C₁₋₄alkoxy.

In a more specific embodiments, for the compounds or pharmaceutically acceptable salts thereof described the thirty-sixth or thirty-seventh embodiment, R¹ is optionally substituted with 1 to 3 substituents independently selected from —CH₃ and —CH₂F.

A thirty-eighth embodiment provides a compound or a pharmaceutically acceptable salt thereof according to any one of embodiments one to thirty-first, wherein:

R¹ is selected from halogen, —C(═O)NR⁴R⁵, C₁₋₄alkyl, C₃₋₆cycloalkyl, —C₁₋₂alkyl-C₃₋₆cycloalkyl, —C₁₋₄alkyl-O—C₁₋₂alkyl, —C₁₋₂alkyl-C₄₋₇heterocycle, —C₁₋₂alkyl-C₅₋₆heteroaryl, —C₁₋₂alkyl-O—C₅₋₆heteroaryl, a fully saturated 4 to 6 membered heterocycle, 5 to 6 membered heteroaryl, phenyl, 5 to 6 membered bridged-carbocyclic ring, 6 to 8 membered fused heterobicyclic ring, 6 to 8 membered spiro heterobicyclic ring, and 6 to 8 membered bridged heterobicyclic ring, wherein R¹ is optionally substituted with 1 to 3 substituents independently selected from halo, hydroxyl, nitrile, C₁₋₄alkyl, C₁₋₄alkoxy, halo-substitutedC₁₋₄alkyl, hydroxyl-substitutedC₁₋₄alkyl, C₁₋₄alkyl-O—C₁₋₂alkyl, —C(═O)OH, —C(═O)—C₁₋₄alkyl, —C(═O)OC₁₋₄alkyl, and 4 to 6 membered heterocycle;

R⁴ and R⁵ are each independently H or C₁₋₄alkyl; and the remaining variables are as described in the first to thirty-first embodiments. In a specific embodiment, R¹ is selected from —Cl, —C(═O)N(CH₃)₂, —CH₃, —CH₂CH₃, —CH₂F, —CHF₂, —CF₂CH₃, —CH₂CH₂CH₂OCH₃, —CF₂CH₂CH₂OCH₃, —CH₂OCH₂CH₃, —CH₂OCH₃, —C(CH₃)₂CH₂OCH₃, —C(CH₃)₂OH, —CH₂CH₂CH₂OH, —CH₂CH₂C(CH₃)₂OH, —CH₂CHFCH₂OH, —CH₂CH₂OCF₃, —CH₂O-(pyridin-2-yl), —CH₂-(1,2,4-triazol-1-yl), —C(CH₃)₂CN, —CH(CH₃)CN, —C(CH₃)₂C(═O)OCH₃, —C(CH₃)₂C(═O)OH, —CH(OH)-cyclopropyl, —CH₂-tetrahydrofuran, —CH(CH₃)-tetrahydrofuran, —CHF-tetrahydrofuran, —CH₂-tetrahydropyranyl, —CH₂-(1,4-dixoan-2-yl), —CH₂-(2-oxopyrrolidin-1-yl), —CH₂-(pyridin-2-yloxy), —CH₂-morpholinyl, C₃₋₆cycloalkyl selected from cyclopropyl, cyclobutyl, cyclopentyl, and cyclohexyl, bicyclo[1.1.1]pentan-1-yl optionally substituted with F, —CH₃, —OCH₃, —CN, CHF₂, —C(CH₃)₂OH, —CH₂OH, and —CH₂OCH₃, 6 to 8 membered fused heterobicyclic ring selected from 3-oxabicyclo[4.1.0]heptan-6-yl, 3-oxabicyclo[3.1.0]hexan-6-yl, and 3-oxabicyclo[3.1.0]hexan-1-yl, 6 to 8 membered fused heterobicyclic ring selected from 5-oxaspiro[2.4]heptan-1-yl, 6-oxaspiro[2.5]octan-1-yl, and 6-oxaspiro[3.4]octan-2-yl, a fully saturated 4 to 6 membered heterocycle selected from tetrahydrofuran-3-yl, tetrahydro-2H-pyran-4-yl, tetrahydro-2H-pyran-3-yl, azetidinyl, tetrahydrothiophen-3-yl, pyrrolidin-3-yl, 2-oxopyrrolidin-3-yl, piperidin-4-yl, 1-methyl-6-oxopiperidin-2-yl, morpholinyl, and 1,4-dioxan-2-yl, phenyl optionally substituted with —F, pyrazolyl optionally substituted with 1 or 2 substituents independently selected from —CH₃ and —CHF₂, a 6 to 8 membered bridged heterobicyclic ring selected from 2-oxabicyclo[2.1.1]hexan-4-yl 8-oxabicyclo[3.2.1]octan-3-yl, 2-oxabicyclo[2.2.1]heptan-4-yl, 2-oxabicyclo[2.2.2]octan-4-yl, wherein the C₃₋₆cycloalkyl is optionally substituted with 1 to 2 substituents independently selected from F, —CN, —OH, —OCH₃, —OCH(CH₃)₂, and —CH₂OH, optionally substituted with 1 to 2 substituents independently selected from —CH₃, OCH₂CH₃, —OH, CN, —CH₂CHF₂, —CH₂CH₂OCH₃, —C(═O)CH₃, and oxetan-3-yl, and the 6 to 8 membered bridged heterobicyclic ring is optionally substituted with 1 to 2 substituents independently selected form —CH₃.

In a thirty-ninth embodiment, the invention provides a compound of formula (I′), (I), (Ia), (Ib), (IIa), (IIb), (IIc), (IId), (IIe), (IIIa), (IIIb), (IIIc), (IIId), (IIIe), (IIIf), (IIIg), (IIIh), (IIIi), or (IIIj) or a pharmaceutically acceptable salt thereof, wherein:

R¹ is selected from halogen, —C(═O)NR⁴R⁵, C₁₋₄alkyl, C₃₋₆cycloalkyl, —C₁₋₂alkyl-C₃₋₆cycloalkyl, —C₁₋₄alkyl-O—C₁₋₂alkyl, —C₁₋₂alkyl-C₄₋₇heterocycle, —C₁₋₂alkyl-C₅₋₆heteroaryl, —C₁₋₂alkyl-O—C₅₋₆heteroaryl, a fully saturated 4 to 6 membered heterocycle, 5 to 6 membered heteroaryl, phenyl, 5 to 6 membered bridged-carbocyclic ring, 6 to 8 membered fused heterobicyclic ring, 6 to 8 membered spiro heterobicyclic ring, and 6 to 8 membered bridged heterobicyclic ring, wherein R¹ is optionally substituted with 1 to 3 substituents independently selected from halo, hydroxyl, nitrile, C₁₋₄alkyl, C₁₋₄alkoxy, halo-substitutedC₁₋₄alkyl, hydroxyl-substitutedC₁₋₄alkyl, C₁₋₄alkyl-O—C₁₋₂alkyl, —C(═O)OH, —C(═O)—C₁₋₄alkyl, —C(═O)OC₁₋₄alkyl, and 4 to 6 membered heterocycle;

R² is hydrogen, C₁₋₄ alkyl or halogen;

R³ is selected from pyridyl, oxazolyl, pyrazinyl, oxadiazoyl, thiazolyl, pyrazolyl, imidazolyl, pyridinyl-2(1H)-one, phenyl, an 8 to 10 membered fused bicyclic heteroaryl and an 8 to 10 membered fused heterobicyclic ring, each of which is optionally substituted with 1 or 2 substituents independently selected from the group consisting halo, halo-substitutedC₁₋₄ alkyl, —NR¹¹R¹², C₁₋₄alkoxy and C₁₋₄ alkyl;

R⁴ and R⁵ are each independently H or C₁₋₄alkyl;

R⁸ is C₁₋₄alkyl, halo-substituted C₁₋₄alkyl, hydroxy-substituted-C₁₋₄ alkyl, halogen, nitrile, NR⁴R⁵, or —OR⁹;

R⁹ is hydrogen or an optionally substituted C₁₋₅alkyl having 1 to 3 substituents independently selected from halogen, hydroxyl, NR⁴R⁵, C₁₋₄alkoxy, phenyl and a 4 to 7 membered partially or fully saturated heterocycle containing 1 or 2 heteroatoms selected from nitrogen and oxygen, wherein said phenyl and 4 to 7 membered heterocycle may be optionally substituted with 1 to 3 R¹⁰; and

R¹⁰ is independently selected from oxo, halo, halo-substitutedC₁₋₄ alkyl and C₁₋₄ alkyl; and the remaining variables are as defined in the first to nineteenth embodiments.

In a fortieth embodiment, the invention provides a compound of formula (I′), (I), (Ia), (Ib), (IIa), (IIb), (IIc), (IId), (IIe), (IIIa), (IIIb), (IIc), (IIId), (IIIe), (IIIf), (IIIg), (IIIh), (IIIi), or (IIIj) or a pharmaceutically acceptable salt thereof, wherein:

R¹ is selected from C₃₋₆cycloalkyl, a fully saturated 4 to 6 membered heterocycle containing 1 to 2 heteroatoms independently selected from nitrogen and oxygen, 5 to 6 membered bridged-carbocyclic ring, 6 to 8 membered spiro heterobicyclic ring containing 1 to 2 heteroatoms independently selected from nitrogen and oxygen, and 6 to 8 membered bridged heterobicyclic ring containing 1 to 2 heteroatoms independently selected from nitrogen and oxygen, wherein R¹ is optionally substituted with 1 to 3 substituents independently selected from halo, hydroxyl, nitrile, C₁₋₄alkyl, C₁₋₄alkoxy, halo-substitutedC₁₋₄alkyl, hydroxyl-substitutedC₁₋₄alkyl, C₁₋₄alkyl-O—C₁₋₂alkyl, —C(═O)OH, —C(═O)—C₁₋₄alkyl, and —C(═O)OC₁₋₄alkyl;

R² is hydrogen, C₁₋₄ alkyl or halogen;

R³ is selected from pyridyl, oxazolyl, pyrazinyl, oxadazoyl, thiazolyl, pyrazolyl, imidazolyl, pyridinyl-2(1H)-one, phenyl, an 8 to 10 membered fused bicyclic heteroaryl and an 8 to 10 membered fused heterobicyclic ring, wherein R³ is optionally substituted with 1 or 2 substituents independently selected from the group consisting halo, halo-substitutedC₁₋₄ alkyl, —NR¹¹R¹², C₁₋₄alkoxy and C₁₋₄ alkyl;

R⁴ and R⁵ are each independently H or C₁₋₄alkyl;

R⁸ is C₁₋₄alkyl, halo-substituted C₁₋₄alkyl, halogen, or —OR⁹;

R⁹ is C₁₋₅alkyl having 1 to 3 substituents independently selected from halogen; and the remaining variables are as defined in the first to nineteenth embodiments.

In a forty-first embodiment, the invention provides a compound of formula (I′), (I), (Ia), (Ib), (IIa), (IIb), (IIc), (IId), (IIe), (IIIa), (IIIb), (IIIc), (IIId), (IIIe), (IIIf), (IIIg), (IIIh), (IIIi), or (IIIj) or a pharmaceutically acceptable salt thereof, wherein:

R¹ is a C₃₋₆cycloalkyl selected from cyclopropyl, cyclobutyl, cyclopentyl, and cyclohexyl, bicyclo[1.1.1]pentan-1-yl, a 6 to 8 membered fused heterobicyclic ring selected from 3-oxabicyclo[4.1.0]heptan-6-yl, 3-oxabicyclo[3.1.0]hexan-6-yl, and 3-oxabicyclo[3.1.0]hexan-1-yl, a 6 to 8 membered fused heterobicyclic ring selected from 5-oxaspiro[2.4]heptan-1-yl, 6-oxaspiro[2.5]octan-1-yl, and 6-oxaspiro[3.4]octan-2-yl, or a fully saturated 4 to 6 membered heterocycle selected from tetrahydrofuran-3-yl, tetrahydro-2H-pyran-4-yl, tetrahydro-2H-pyran-3-yl, azetidinyl, tetrahydrothiophen-3-yl, pyrrolidin-3-yl, 2-oxopyrrolidin-3-yl, piperidin-4-yl, 1-methyl-6-oxopiperidin-2-yl, morpholinyl, and 1,4-dioxan-2-yl, wherein R¹ is optionally substituted with 1 to 3 substituents independently selected from halo, hydroxyl, nitrile, C₁₋₄alkyl, C₁₋₄alkoxy, halo-substitutedC₁₋₄alkyl, hydroxyl-substitutedC₁₋₄alkyl, C₁₋₄alkyl-O—C₁₋₂alkyl, —C(═O)OH, —C(═O)—C₁₋₄alkyl, and —C(═O)OC₁₋₄alkyl;

R² is hydrogen, C₁₋₄ alkyl or halogen;

R³ is selected from pyridyl, oxazolyl, pyrazinyl, oxadazoyl, thiazolyl, pyrazolyl, imidazolyl, pyridinyl-2(1H)-one, phenyl, 2,3-dihydrofuro[2,3-b]pyridine, 5,6,7,8-tetrahydroindolizine, 2,3-dihydrothieno[3,4-b][1,4]dioxine, 6,7-dihydro-5H-cyclopenta[b]pyridine, 2,3-dihydrobenzofuran, 2,3-dihydro-1H-pyrrolizine, pyrazolo[1,5-a]pyridine, pyrazolo[1,5-a]pyrimidine, and [1,2,4]triazolo[1,5-a]pyridine, wherein R³ is optionally substituted with 1 or 2 substituents independently selected from the group consisting halo, halo-substitutedC₁₋₄ alkyl, —NR¹¹R¹², C₁₋₄alkoxy and C₁₋₄ alkyl;

R⁴ and R⁵ are each independently H or C₁₋₄alkyl;

R⁸ is C₁₋₄alkyl, halo-substituted C₁₋₄alkyl, halogen, or —OR⁹.

R⁹ is C₁₋₅alkyl having 1 to 3 substituents independently selected from halogen; and the remaining variables are as defined in the first to nineteenth embodiments.

In a forty-second embodiment, the invention provides a compound represented by formula (V), (VI), (VII) or (VIII):

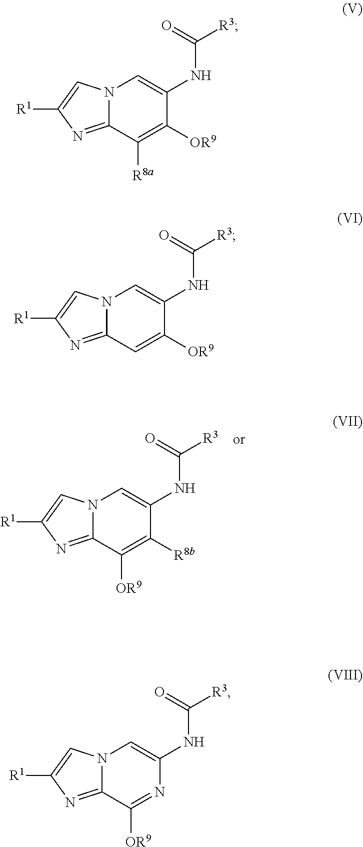

or a pharmaceutically acceptable salt thereof, wherein:

R¹ is a 6 to 8 membered bridged-heterocyclic ring containing 1 to 2 heteroatoms independently selected from oxygen and nitrogen, wherein the 6 to 8 membered bridged-heterocyclic ring is optionally substituted with 1 or 2 substituents independently selected from C₁₋₄alkyl, halogen, halo-substitutedC₁₋₄ alkyl, hydroxyl and C₁₋₄alkoxy;

R³ is a pyridyl, pyridinyl-2(1H)-one, a 9 to 10 membered fused heterobicyclic ring having 1 to 2 heteroatoms independently selected from oxygen and nitrogen, or a 9 to 10 membered fused bicyclic heteroaryl having 1 to 3 heteroatoms independently selected from oxygen and nitrogen, wherein the pyridyl, the 9 to 10 membered fused heterobicyclic ring, and the 9 to 10 membered fused bicyclic heteroaryl represented by R³ are each optionally substituted with 1 to 2 substituents independently selected from C₁₋₄alkyl, halogen, halo-substitutedC₁₋₄ alkyl, hydroxyl and C₁₋₄alkoxy;

R^{8b} is H or halogen; and

R⁹ is C₁₋₅alkyl.

In a specific embodiment, for the compounds or pharmaceutically acceptable salts thereof described in the forty-second embodiments,

R¹ is a 6 or 7 membered bridged-heterocyclic ring having an oxygen atom, optionally substituted with a C₁₋₄alkyl;

R³ is pyridyl, pyridinyl-2(1H)-one, 9 membered fused heterobicyclic ring having an oxygen atom or a 9 membered fused bicyclic heteroaryl having 2 or 3 nitrogen atoms, wherein the pyridyl, the 9 membered fused heterobicyclic ring, and the 9 membered fused bicyclic heteroaryl represented by R³ are each optionally substituted with 1 to 2 substituents independently selected from halogen, halo-substitutedC₁₋₄ alkyl and C₁₋₄alkoxy;

R^{8b} is H or F; and

R⁹ is C₁₋₄alkyl; and the remaining variables are as defined in the forty-second embodiment.

In another specific embodiment, for the compounds or pharmaceutically acceptable salts thereof described in the forty-second embodiment, R¹ is selected from 2-oxabicyclo[2.1.1]hexan-4-yl 8-oxabicyclo[3.2.1]octan-3-yl, 2-oxabicyclo[2.2.1]heptan-4-yl, 2-oxabicyclo[2.2.2]octan-4-yl, 2-oxabicyclo[3.1.1]heptan-5-yl, each of which is optionally substituted with C₁₋₄alkyl;

R³ is selected from 2-pyridyl, 3-pyridyl, pyridinyl-2(1H)-one, 2,3-dihydrobenzofuran, pyrazolo[1,5-a]pyridine, and [1,2,4]triazolo[1,5-a]pyridine, each of which is optionally substituted with 1 to 2 substituents independently selected from halo, halo-substitutedC_₄ alkyl and C₁₋₄alkoxy;

R⁹ is C₁₋₄alkyl; and the remaining variables are as defined in the forty-second embodiment.

A forty-third embodiment of the invention provides a compound selected from the group consisting of:

| Ex. | |
|---|---|
| 1 | 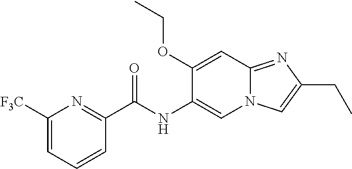 [SMILES: CCOc1cc2nc(CC)cn2cc1NC(=O)c1cccc(C(F)(F)F)n1] |
| 2 | 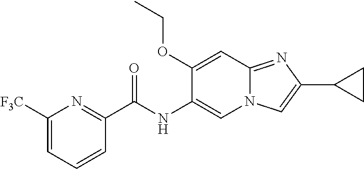 [SMILES: CCOc1cc2nc(C3CC3)cn2cc1NC(=O)c1cccc(C(F)(F)F)n1] |
| 3 | 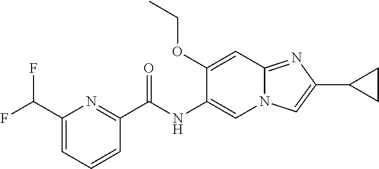 [SMILES: CCOc1cc2nc(C3CC3)cn2cc1NC(=O)c1cccc(C(F)F)n1] |
| 4 | 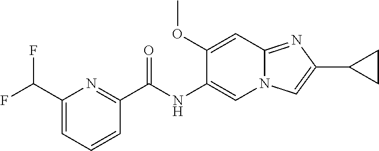 [SMILES: COc1cc2nc(C3CC3)cn2cc1NC(=O)c1cccc(C(F)F)n1] |
| 5 | 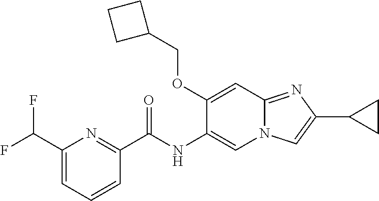 [SMILES: O=C(Nc1cn2cc(C3CC3)nc2cc1OCC1CCC1)c1cccc(C(F)F)n1] |
| 6 | 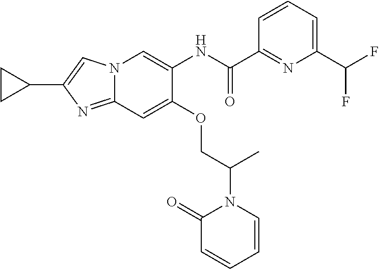 [SMILES: CC(COc1cc2nc(C3CC3)cn2cc1NC(=O)c1cccc(C(F)F)n1)n1ccccc1=O] |
| 7 | 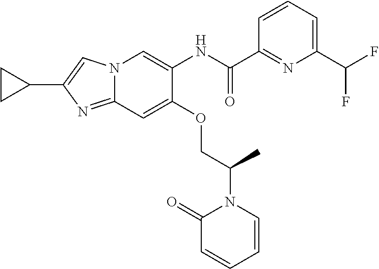 [SMILES: C[C@H](COc1cc2nc(C3CC3)cn2cc1NC(=O)c1cccc(C(F)F)n1)n1ccccc1=O] |
| 8 | 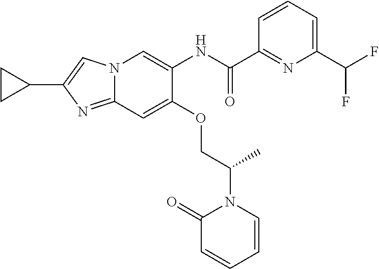 [SMILES: C[C@@H](COc1cc2nc(C3CC3)cn2cc1NC(=O)c1cccc(C(F)F)n1)n1ccccc1=O] |
| 9 | 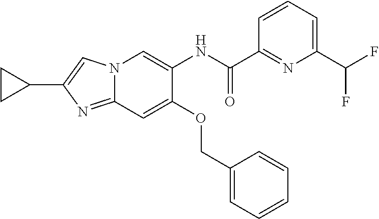 [SMILES: O=C(Nc1cn2cc(C3CC3)nc2cc1OCc1ccccc1)c1cccc(C(F)F)n1] |
| 10 | 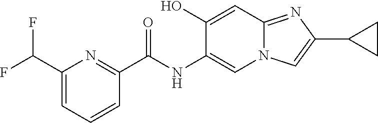 [SMILES: O=C(Nc1cn2cc(C3CC3)nc2cc1O)c1cccc(C(F)F)n1] |
| 11 | 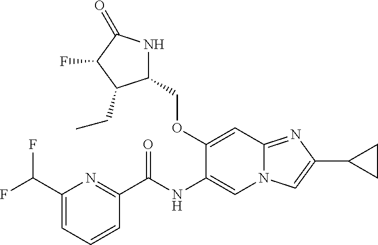 [SMILES: CC[C@@H]1[C@H](F)C(=O)N[C@@H]1COc1cc2nc(C3CC3)cn2cc1NC(=O)c1cccc(C(F)F)n1] |
| 12 | 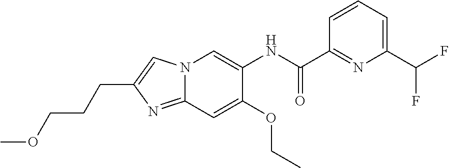 [SMILES: CCOc1cc2nc(CCCOC)cn2cc1NC(=O)c1cccc(C(F)F)n1] |
| 13 | 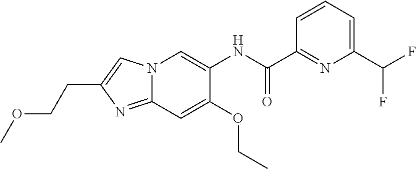 [SMILES: CCOc1cc2nc(CCOC)cn2cc1NC(=O)c1cccc(C(F)F)n1] |
| 14 | 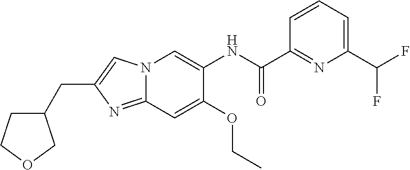 [SMILES: CCOc1cc2nc(CC3CCOC3)cn2cc1NC(=O)c1cccc(C(F)F)n1] |
| 15 | 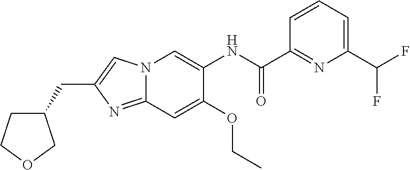 [SMILES: CCOc1cc2nc(C[C@H]3CCOC3)cn2cc1NC(=O)c1cccc(C(F)F)n1] |
| 16 | 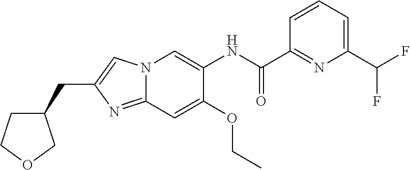 [SMILES: CCOc1cc2nc(C[C@@H]3CCOC3)cn2cc1NC(=O)c1cccc(C(F)F)n1] |
| 17 | 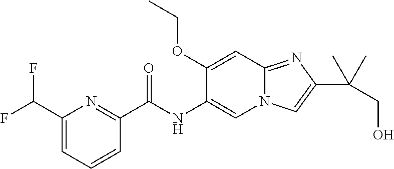 [SMILES: CCOc1cc2nc(C(C)(C)CO)cn2cc1NC(=O)c1cccc(C(F)F)n1] |
| 18 | 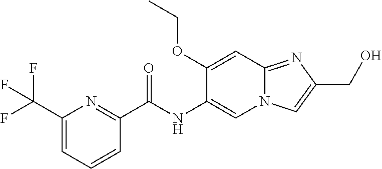 [SMILES: CCOc1cc2nc(CO)cn2cc1NC(=O)c1cccc(C(F)(F)F)n1] |
| 19 | 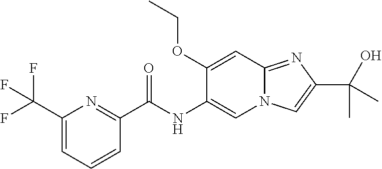 [SMILES: CCOc1cc2nc(C(C)(C)O)cn2cc1NC(=O)c1cccc(C(F)(F)F)n1] |
| 20 | 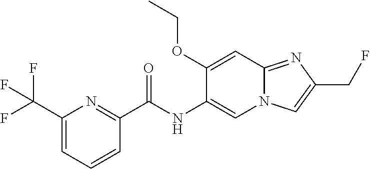 [SMILES: CCOc1cc2nc(CF)cn2cc1NC(=O)c1cccc(C(F)(F)F)n1] |
| 21 | 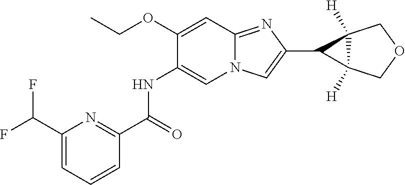 |
| 22 | 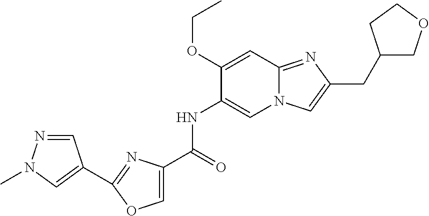 [SMILES: CCOc1cc2nc(CC3CCOC3)cn2cc1NC(=O)c1coc(-c2cnn(C)c2)n1] |
| 23 | 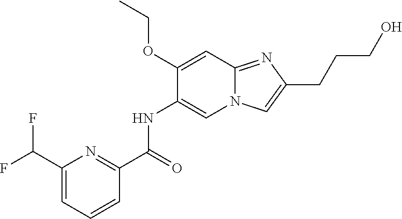 [SMILES: CCOc1cc2nc(CCCO)cn2cc1NC(=O)c1cccc(C(F)F)n1] |
| 24 | 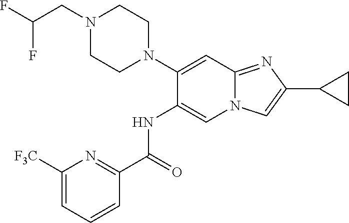 [SMILES: O=C(Nc1cn2cc(C3CC3)nc2cc1N1CCN(CC(F)F)CC1)c1cccc(C(F)(F)F)n1] |
| 25 | 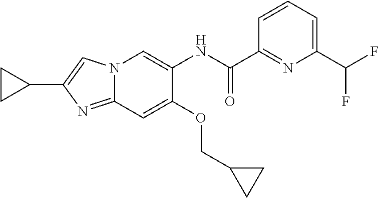 [SMILES: O=C(Nc1cn2cc(C3CC3)nc2cc1OCC1CC1)c1cccc(C(F)F)n1] |
| 26 | 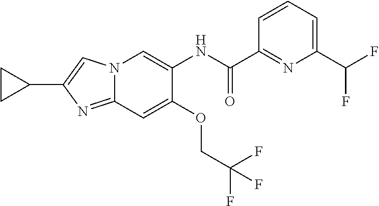 [SMILES: O=C(Nc1cn2cc(C3CC3)nc2cc1OCC(F)(F)F)c1cccc(C(F)F)n1] |
| 27 | 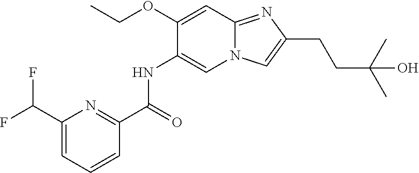 [SMILES: CCOc1cc2nc(CCC(C)(C)O)cn2cc1NC(=O)c1cccc(C(F)F)n1] |
| 28 | 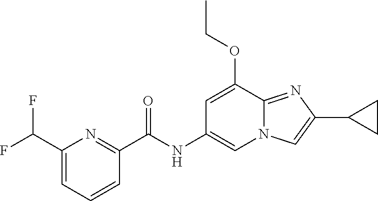 [SMILES: CCOc1cc(NC(=O)c2cccc(C(F)F)n2)cn2cc(C3CC3)nc12] |
| 29 | 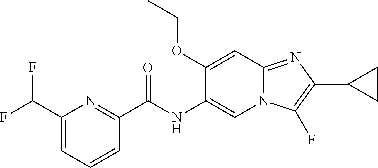 [SMILES: CCOc1cc2nc(C3CC3)c(F)n2cc1NC(=O)c1cccc(C(F)F)n1] |
| 30 | 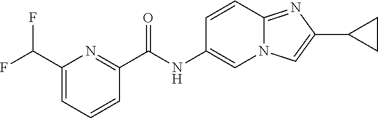 [SMILES: O=C(Nc1ccc2nc(C3CC3)cn2c1)c1cccc(C(F)F)n1] |
| 31 | 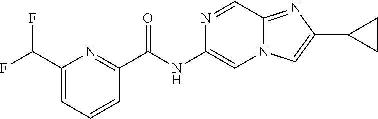 [SMILES: O=C(Nc1cn2cc(C3CC3)nc2cn1)c1cccc(C(F)F)n1] |
| 32 | 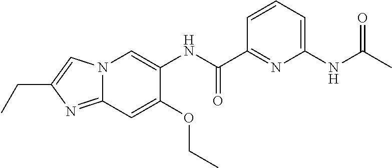 [SMILES: CCOc1cc2nc(CC)cn2cc1NC(=O)c1cccc(NC(C)=O)n1] |
| 33 | 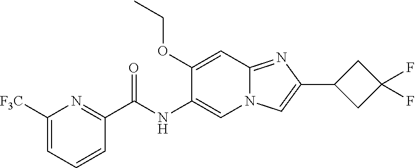 [SMILES: CCOc1cc2nc(C3CC(F)(F)C3)cn2cc1NC(=O)c1cccc(C(F)(F)F)n1] |
| 34 | 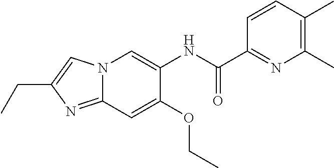 [SMILES: CCOc1cc2nc(CC)cn2cc1NC(=O)c1ccc(C)c(C)n1] |
| 35 | 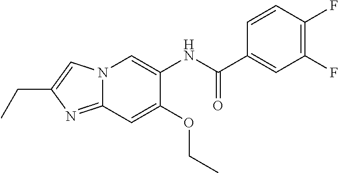 [SMILES: CCOc1cc2nc(CC)cn2cc1NC(=O)c1ccc(F)c(F)c1] |
| 36 | 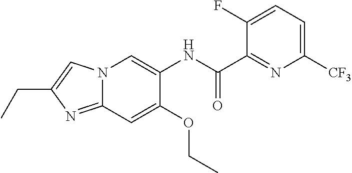 [SMILES: CCOc1cc2nc(CC)cn2cc1NC(=O)c1nc(C(F)(F)F)ccc1F] |
| 37 | 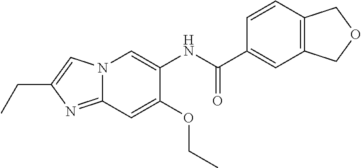 [SMILES: CCOc1cc2nc(CC)cn2cc1NC(=O)c1ccc2c(c1)COC2] |
| 38 | 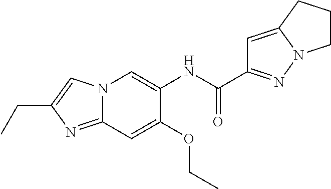 [SMILES: CCOc1cc2nc(CC)cn2cc1NC(=O)c1cc2n(n1)CCC2] |
| 39 | 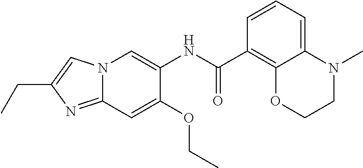 [SMILES: CCOc1cc2nc(CC)cn2cc1NC(=O)c1cccc2c1OCCN2C] |
| 40 | 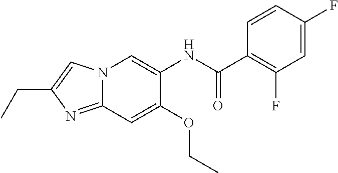 [SMILES: CCOc1cc2nc(CC)cn2cc1NC(=O)c1ccc(F)cc1F] |
| 41 | 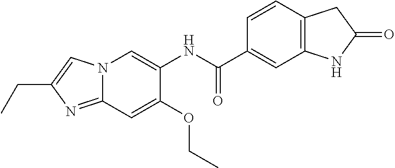 [SMILES: CCOc1cc2nc(CC)cn2cc1NC(=O)c1ccc2c(c1)NC(=O)C2] |
| 42 | 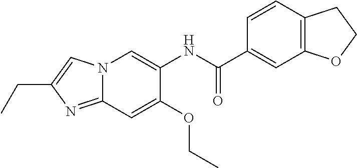 [SMILES: CCOc1cc2nc(CC)cn2cc1NC(=O)c1ccc2c(c1)OCC2] |
| 43 | 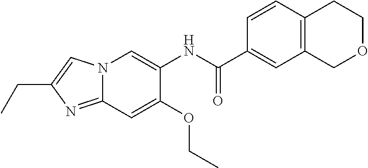 [SMILES: CCOc1cc2nc(CC)cn2cc1NC(=O)c1ccc2c(c1)COCC2] |
| 44 | 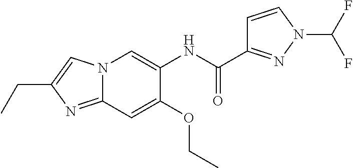 [SMILES: CCOc1cc2nc(CC)cn2cc1NC(=O)c1ccn(C(F)F)n1] |
| 45 | 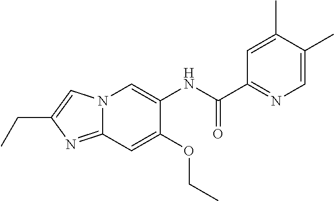 [SMILES: CCOc1cc2nc(CC)cn2cc1NC(=O)c1cc(C)c(C)cn1] |
| 46 | 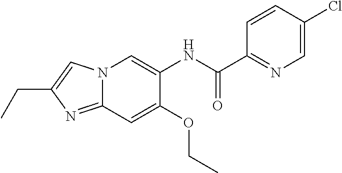 [SMILES: CCOc1cc2nc(CC)cn2cc1NC(=O)c1ccc(Cl)cn1] |
| 47 | 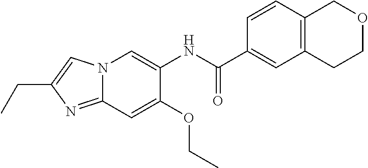 [SMILES: CCOc1cc2nc(CC)cn2cc1NC(=O)c1ccc2c(c1)CCOC2] |
| 48 | 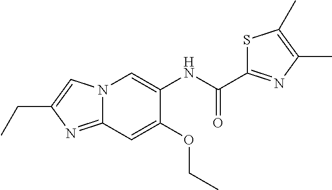 [SMILES: CCOc1cc2nc(CC)cn2cc1NC(=O)c1nc(C)c(C)s1] |
| 49 | 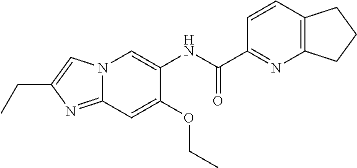 [SMILES: CCOc1cc2nc(CC)cn2cc1NC(=O)c1ccc2c(n1)CCC2] |
| 50 | 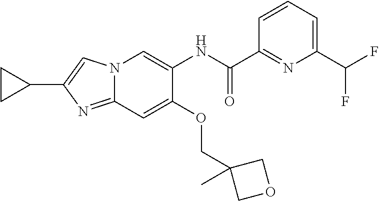 [SMILES: CC1(COc2cc3nc(C4CC4)cn3cc2NC(=O)c2cccc(C(F)F)n2)COC1] |
| 51 | 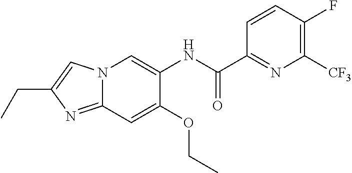 [SMILES: CCOc1cc2nc(CC)cn2cc1NC(=O)c1ccc(F)c(C(F)(F)F)n1] |
| 52 | 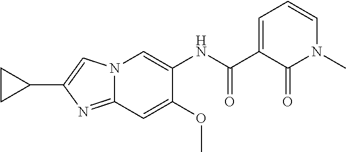 [SMILES: COc1cc2nc(C3CC3)cn2cc1NC(=O)c1cccn(C)c1=O] |
| 53 | 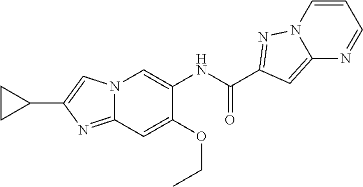 [SMILES: CCOc1cc2nc(C3CC3)cn2cc1NC(=O)c1cc2ncccn2n1] |
| 54 | 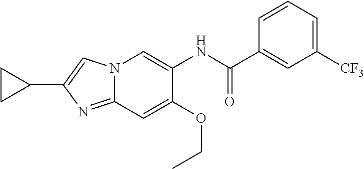 [SMILES: CCOc1cc2nc(C3CC3)cn2cc1NC(=O)c1cccc(C(F)(F)F)c1] |
| 55 | 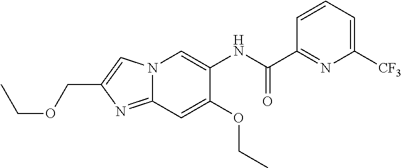 [SMILES: CCOCc1cn2cc(NC(=O)c3cccc(C(F)(F)F)n3)c(OCC)cc2n1] |
| 56 | 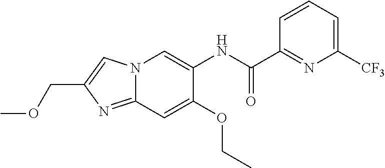 [SMILES: CCOc1cc2nc(COC)cn2cc1NC(=O)c1cccc(C(F)(F)F)n1] |
| 57 | 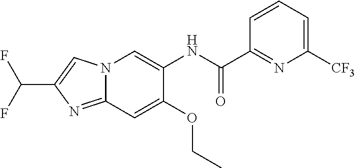 [SMILES: CCOc1cc2nc(C(F)F)cn2cc1NC(=O)c1cccc(C(F)(F)F)n1] |
| 58 | 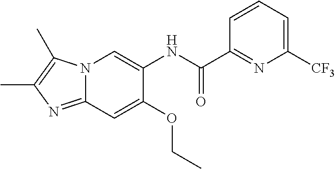 [SMILES: CCOc1cc2nc(C)c(C)n2cc1NC(=O)c1cccc(C(F)(F)F)n1] |
| 59 | 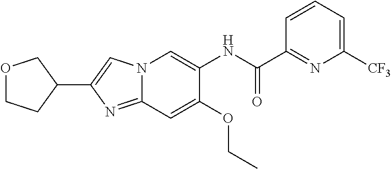 [SMILES: CCOc1cc2nc(C3CCOC3)cn2cc1NC(=O)c1cccc(C(F)(F)F)n1] |
| 60 | 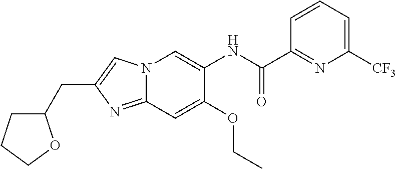 [SMILES: CCOc1cc2nc(CC3CCCO3)cn2cc1NC(=O)c1cccc(C(F)(F)F)n1] |
| 61 | 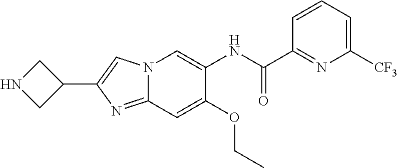 [SMILES: CCOc1cc2nc(C3CNC3)cn2cc1NC(=O)c1cccc(C(F)(F)F)n1] |
| 62 | 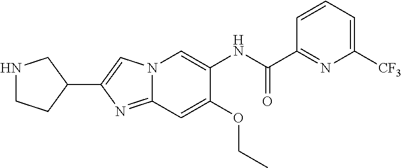 [SMILES: CCOc1cc2nc(C3CCNC3)cn2cc1NC(=O)c1cccc(C(F)(F)F)n1] |
| 63 | 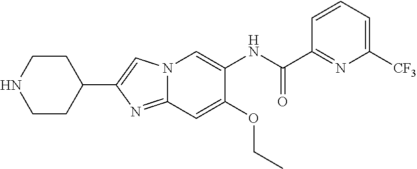 [SMILES: CCOc1cc2nc(C3CCNCC3)cn2cc1NC(=O)c1cccc(C(F)(F)F)n1] |
| 64 | 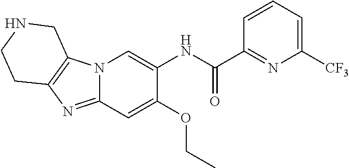 [SMILES: CCOc1cc2nc3c(n2cc1NC(=O)c1cccc(C(F)(F)F)n1)CNCC3] |
| 65 | 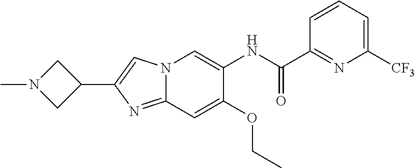 [SMILES: CCOc1cc2nc(C3CN(C)C3)cn2cc1NC(=O)c1cccc(C(F)(F)F)n1] |
| 66 | 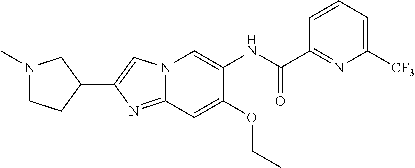 [SMILES: CCOc1cc2nc(C3CCN(C)C3)cn2cc1NC(=O)c1cccc(C(F)(F)F)n1] |
| 67 | 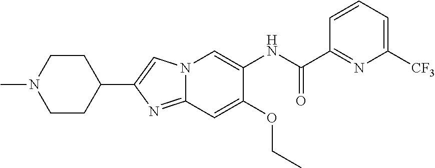 [SMILES: CCOc1cc2nc(C3CCN(C)CC3)cn2cc1NC(=O)c1cccc(C(F)(F)F)n1] |
| 68 | 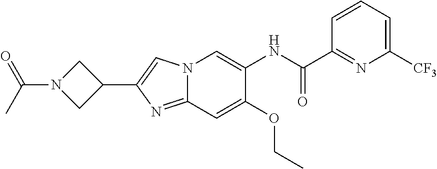 [SMILES: CCOc1cc2nc(C3CN(C(C)=O)C3)cn2cc1NC(=O)c1cccc(C(F)(F)F)n1] |
| 69 | 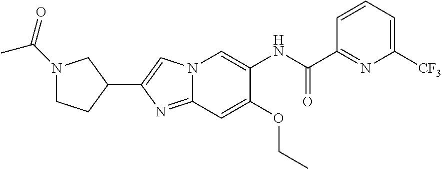 [SMILES: CCOc1cc2nc(C3CCN(C(C)=O)C3)cn2cc1NC(=O)c1cccc(C(F)(F)F)n1] |
| 70 | 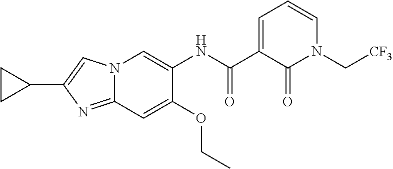 [SMILES: CCOc1cc2nc(C3CC3)cn2cc1NC(=O)c1cccn(CC(F)(F)F)c1=O] |
| 71 | 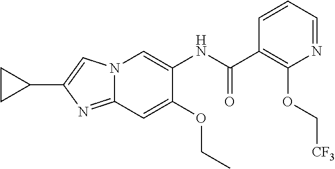 [SMILES: CCOc1cc2nc(C3CC3)cn2cc1NC(=O)c1cccnc1OCC(F)(F)F] |
| 72 | 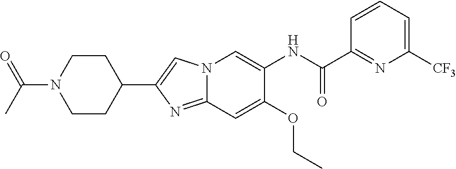 [SMILES: CCOc1cc2nc(C3CCN(C(C)=O)CC3)cn2cc1NC(=O)c1cccc(C(F)(F)F)n1] |
| 73 | 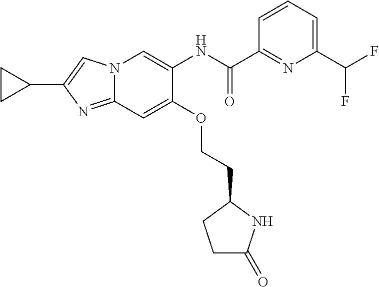 [SMILES: O=C1CC[C@@H](CCOc2cc3nc(C4CC4)cn3cc2NC(=O)c2cccc(C(F)F)n2)N1] |
| 74 | 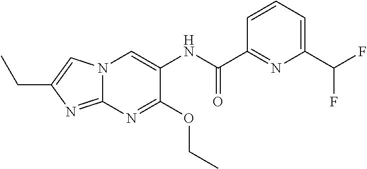 [SMILES: CCOc1nc2nc(CC)cn2cc1NC(=O)c1cccc(C(F)F)n1] |
| 75 | 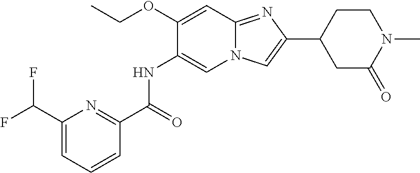 [SMILES: CCOc1cc2nc(C3CCN(C)C(=O)C3)cn2cc1NC(=O)c1cccc(C(F)F)n1] |
| 76 | 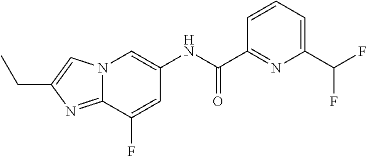 [SMILES: CCc1cn2cc(NC(=O)c3cccc(C(F)F)n3)cc(F)c2n1] |
| 77 | 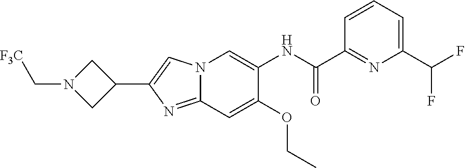 [SMILES: CCOc1cc2nc(C3CN(CC(F)(F)F)C3)cn2cc1NC(=O)c1cccc(C(F)F)n1] |
| 78 | 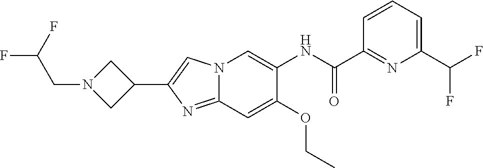 [SMILES: CCOc1cc2nc(C3CN(CC(F)F)C3)cn2cc1NC(=O)c1cccc(C(F)F)n1] |
| 79 | 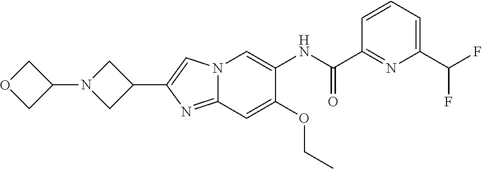 [SMILES: CCOc1cc2nc(C3CN(C4COC4)C3)cn2cc1NC(=O)c1cccc(C(F)F)n1] |
| 80 | 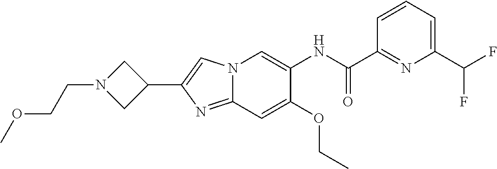 [SMILES: CCOc1cc2nc(C3CN(CCOC)C3)cn2cc1NC(=O)c1cccc(C(F)F)n1] |
| 81 | 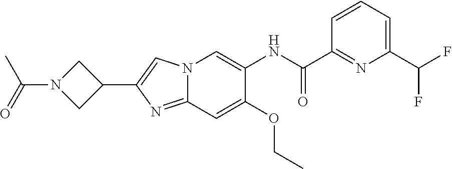 [SMILES: CCOc1cc2nc(C3CN(C(C)=O)C3)cn2cc1NC(=O)c1cccc(C(F)F)n1] |
| 82 | 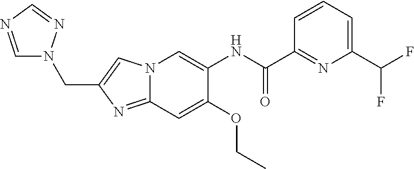 [SMILES: CCOc1cc2nc(Cn3cncn3)cn2cc1NC(=O)c1cccc(C(F)F)n1] |
| 83 | 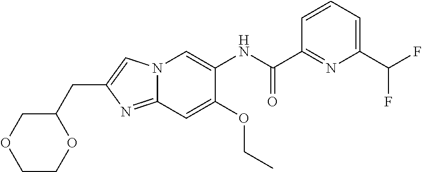 [SMILES: CCOc1cc2nc(CC3COCCO3)cn2cc1NC(=O)c1cccc(C(F)F)n1] |
| 84 | 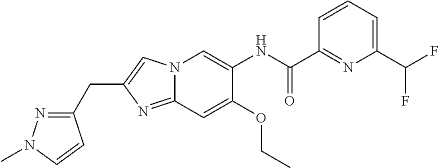 [SMILES: CCOc1cc2nc(Cc3ccn(C)n3)cn2cc1NC(=O)c1cccc(C(F)F)n1] |
| 85 | 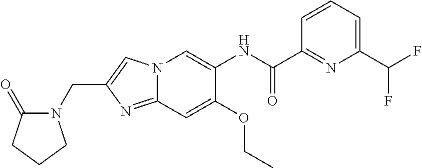 [SMILES: CCOc1cc2nc(CN3CCCC3=O)cn2cc1NC(=O)c1cccc(C(F)F)n1] |
| 86 | 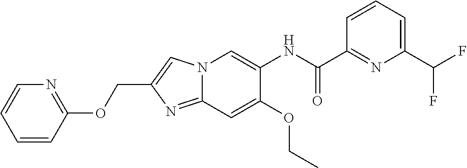 [SMILES: CCOc1cc2nc(COc3ccccn3)cn2cc1NC(=O)c1cccc(C(F)F)n1] |
| 87 | 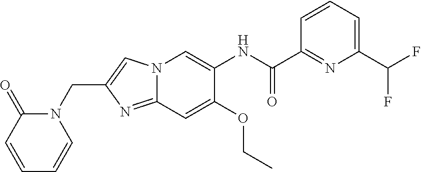 [SMILES: CCOc1cc2nc(Cn3ccccc3=O)cn2cc1NC(=O)c1cccc(C(F)F)n1] |
| 88 | 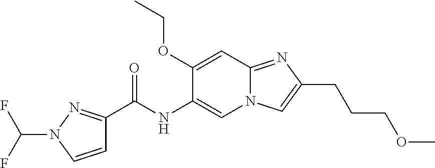 [SMILES: CCOc1cc2nc(CCCOC)cn2cc1NC(=O)c1ccn(C(F)F)n1] |
| 89 | 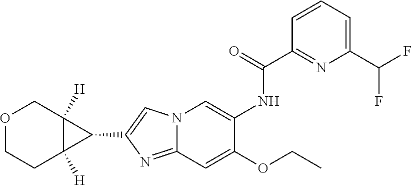 [SMILES: CCOc1cc2nc([C@H]3[C@@H]4COCC[C@@H]43)cn2cc1NC(=O)c1cccc(C(F)F)n1] |
| 90 | 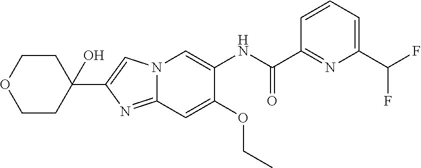 [SMILES: CCOc1cc2nc(C3(O)CCOCC3)cn2cc1NC(=O)c1cccc(C(F)F)n1] |
| 91 | 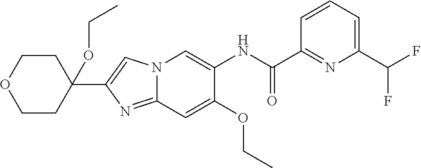 [SMILES: CCOc1cc2nc(C3(OCC)CCOCC3)cn2cc1NC(=O)c1cccc(C(F)F)n1] |
| 92 | 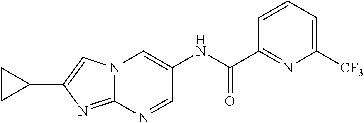 [SMILES: O=C(Nc1cnc2nc(C3CC3)cn2c1)c1cccc(C(F)(F)F)n1] |
| 93 | 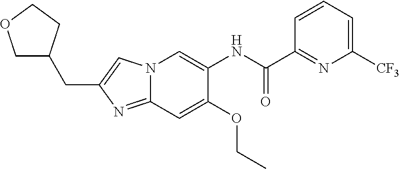 [SMILES: CCOc1cc2nc(CC3CCOC3)cn2cc1NC(=O)c1cccc(C(F)(F)F)n1] |
| 94 | 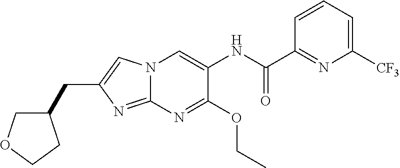 |
| 95 | 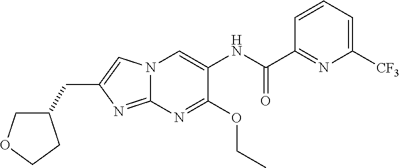 |
| 96 | 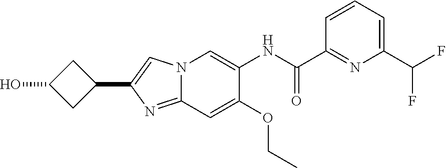 [SMILES: CCOc1cc2nc([C@H]3C[C@H](O)C3)cn2cc1NC(=O)c1cccc(C(F)F)n1] |
| 97 | 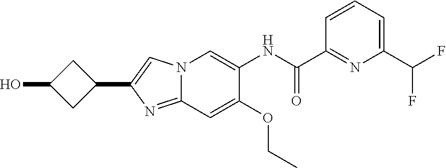 [SMILES: CCOc1cc2nc([C@H]3C[C@@H](O)C3)cn2cc1NC(=O)c1cccc(C(F)F)n1] |
| 98 | 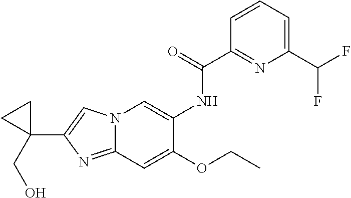 [SMILES: CCOc1cc2nc(C3(CO)CC3)cn2cc1NC(=O)c1cccc(C(F)F)n1] |
| 99 | 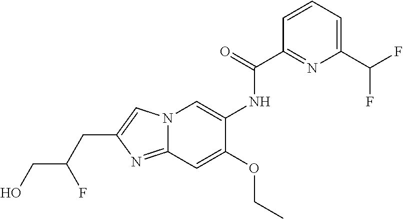 [SMILES: CCOc1cc2nc(CC(F)CO)cn2cc1NC(=O)c1cccc(C(F)F)n1] |
| 100 | 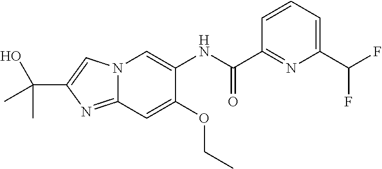 [SMILES: CCOc1cc2nc(C(C)(C)O)cn2cc1NC(=O)c1cccc(C(F)F)n1] |
| 101 | 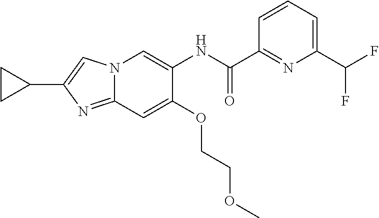 [SMILES: COCCOc1cc2nc(C3CC3)cn2cc1NC(=O)c1cccc(C(F)F)n1] |
| 102 | 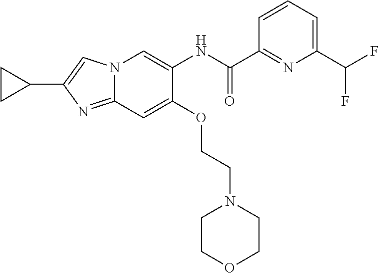 [SMILES: O=C(Nc1cn2cc(C3CC3)nc2cc1OCCN1CCOCC1)c1cccc(C(F)F)n1] |
| 103 | 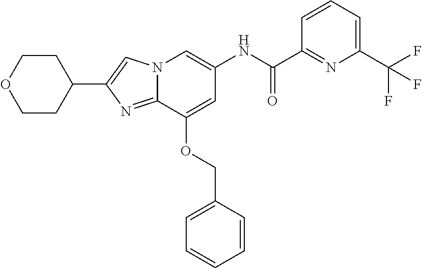 [SMILES: O=C(Nc1cc(OCc2ccccc2)c2nc(C3CCOCC3)cn2c1)c1cccc(C(F)(F)F)n1] |
| 104 | 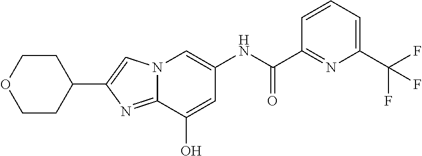 [SMILES: O=C(Nc1cc(O)c2nc(C3CCOCC3)cn2c1)c1cccc(C(F)(F)F)n1] |
| 105 | 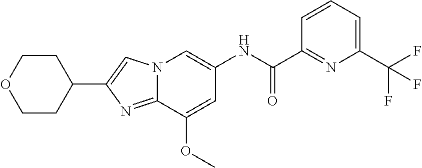 [SMILES: COc1cc(NC(=O)c2cccc(C(F)(F)F)n2)cn2cc(C3CCOCC3)nc12] |
| 106 | 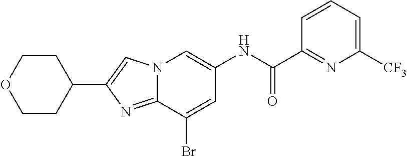 [SMILES: O=C(Nc1cc(Br)c2nc(C3CCOCC3)cn2c1)c1cccc(C(F)(F)F)n1] |
| 107 | 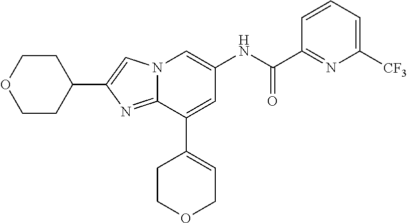 [SMILES: O=C(Nc1cc(C2=CCOCC2)c2nc(C3CCOCC3)cn2c1)c1cccc(C(F)(F)F)n1] |
| 108 | 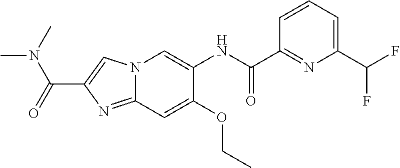 [SMILES: CCOc1cc2nc(C(=O)N(C)C)cn2cc1NC(=O)c1cccc(C(F)F)n1] |
| 109 | 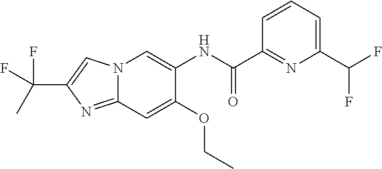 [SMILES: CCOc1cc2nc(C(C)(F)F)cn2cc1NC(=O)c1cccc(C(F)F)n1] |
| 110 | 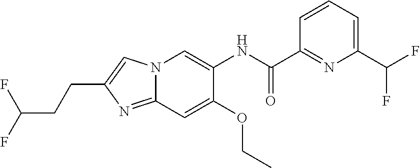 [SMILES: CCOc1cc2nc(CCC(F)F)cn2cc1NC(=O)c1cccc(C(F)F)n1] |
| 111 | 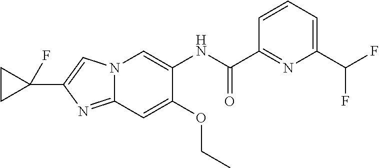 [SMILES: CCOc1cc2nc(C3(F)CC3)cn2cc1NC(=O)c1cccc(C(F)F)n1] |
| 112 | 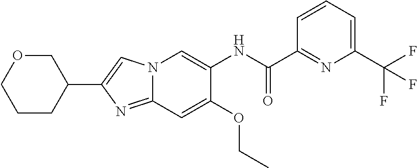 [SMILES: CCOc1cc2nc(C3CCCOC3)cn2cc1NC(=O)c1cccc(C(F)(F)F)n1] |
| 113 | 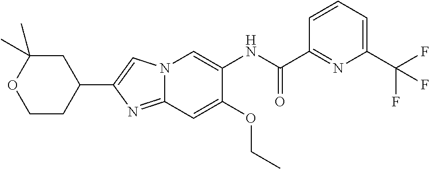 [SMILES: CCOc1cc2nc(C3CCOC(C)(C)C3)cn2cc1NC(=O)c1cccc(C(F)(F)F)n1] |
| 114 | 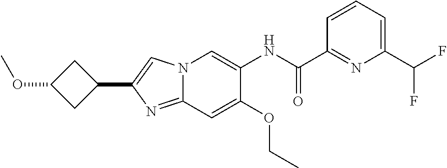 [SMILES: CCOc1cc2nc([C@H]3C[C@H](OC)C3)cn2cc1NC(=O)c1cccc(C(F)F)n1] |
| 115 | 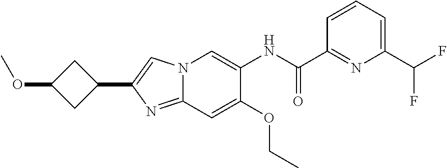 [SMILES: CCOc1cc2nc([C@H]3C[C@@H](OC)C3)cn2cc1NC(=O)c1cccc(C(F)F)n1] |
| 116 | 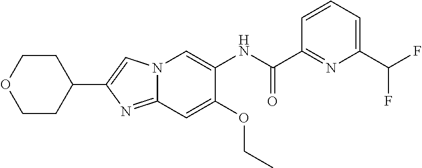 [SMILES: CCOc1cc2nc(C3CCOCC3)cn2cc1NC(=O)c1cccc(C(F)F)n1] |
| 117 | 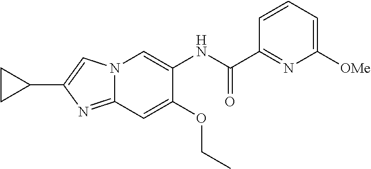 [SMILES: CCOc1cc2nc(C3CC3)cn2cc1NC(=O)c1cccc(OC)n1] |
| 118 | 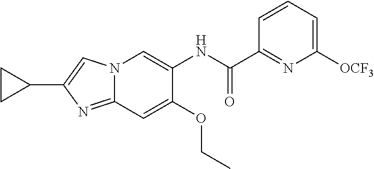 [SMILES: CCOc1cc2nc(C3CC3)cn2cc1NC(=O)c1cccc(OC(F)(F)F)n1] |
| 119 | 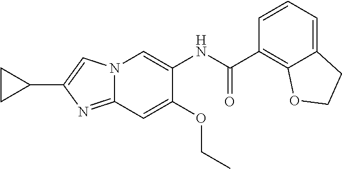 [SMILES: CCOc1cc2nc(C3CC3)cn2cc1NC(=O)c1cccc2c1OCC2] |
| 120 | 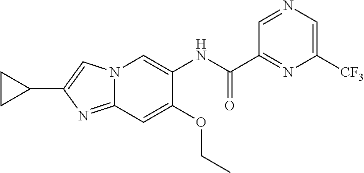 [SMILES: CCOc1cc2nc(C3CC3)cn2cc1NC(=O)c1cncc(C(F)(F)F)n1] |
| 121 | 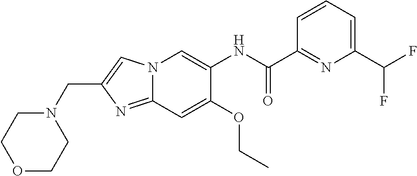 [SMILES: CCOc1cc2nc(CN3CCOCC3)cn2cc1NC(=O)c1cccc(C(F)F)n1] |
| 122 | 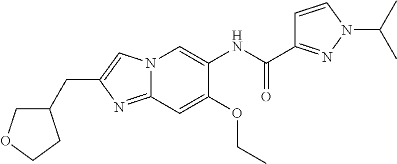 [SMILES: CCOc1cc2nc(CC3CCOC3)cn2cc1NC(=O)c1ccn(C(C)C)n1] |
| 123 | 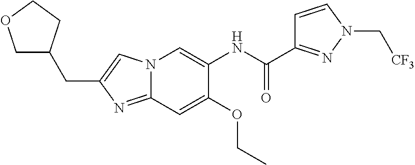 [SMILES: CCOc1cc2nc(CC3CCOC3)cn2cc1NC(=O)c1ccn(CC(F)(F)F)n1] |
| 124 | 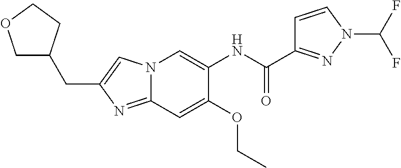 [SMILES: CCOc1cc2nc(CC3CCOC3)cn2cc1NC(=O)c1ccn(C(F)F)n1] |
| 125 | 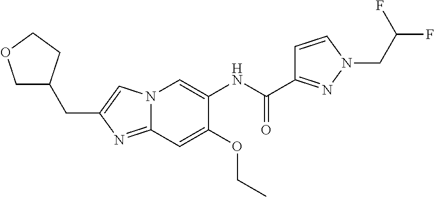 [SMILES: CCOc1cc2nc(CC3CCOC3)cn2cc1NC(=O)c1ccn(CC(F)F)n1] |
| 126 | 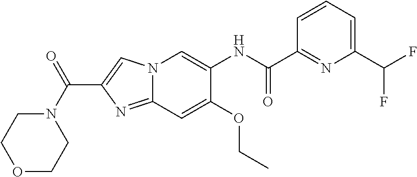 [SMILES: CCOc1cc2nc(C(=O)N3CCOCC3)cn2cc1NC(=O)c1cccc(C(F)F)n1] |
| 127 | 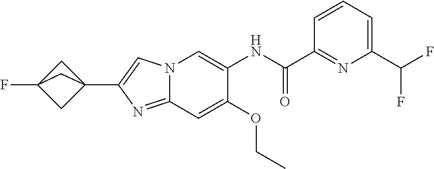 [SMILES: CCOc1cc2nc(C34CC(F)(C3)C4)cn2cc1NC(=O)c1cccc(C(F)F)n1] |
| 128 | 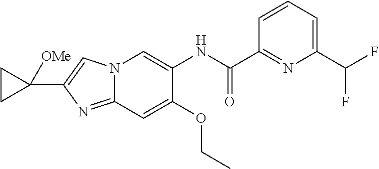 [SMILES: CCOc1cc2nc(C3(OC)CC3)cn2cc1NC(=O)c1cccc(C(F)F)n1] |
| 129 | 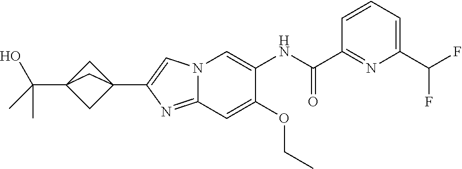 [SMILES: CCOc1cc2nc(C34CC(C(C)(C)O)(C3)C4)cn2cc1NC(=O)c1cccc(C(F)F)n1] |
| 130 | 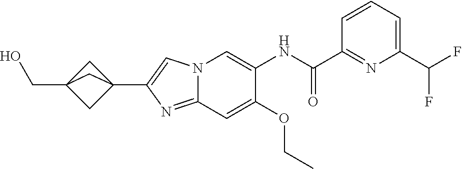 [SMILES: CCOc1cc2nc(C34CC(CO)(C3)C4)cn2cc1NC(=O)c1cccc(C(F)F)n1] |
| 131 | 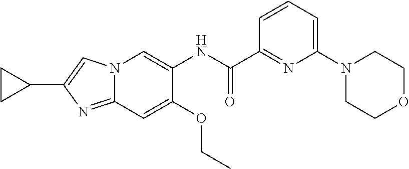 [SMILES: CCOc1cc2nc(C3CC3)cn2cc1NC(=O)c1cccc(N2CCOCC2)n1] |
| 132 | 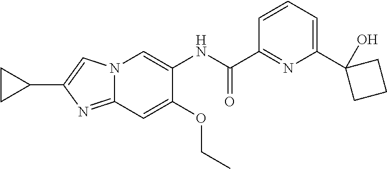 [SMILES: CCOc1cc2nc(C3CC3)cn2cc1NC(=O)c1cccc(C2(O)CCC2)n1] |
| 133 | 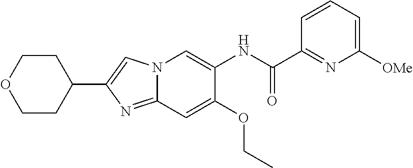 [SMILES: CCOc1cc2nc(C3CCOCC3)cn2cc1NC(=O)c1cccc(OC)n1] |
| 134 | 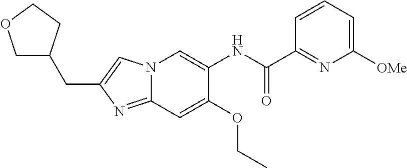 [SMILES: CCOc1cc2nc(CC3CCOC3)cn2cc1NC(=O)c1cccc(OC)n1] |
| 135 | 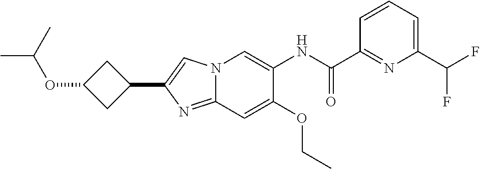 [SMILES: CCOc1cc2nc([C@H]3C[C@H](OC(C)C)C3)cn2cc1NC(=O)c1cccc(C(F)F)n1] |
| 136 | 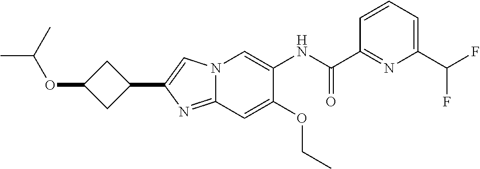 [SMILES: CCOc1cc2nc([C@H]3C[C@@H](OC(C)C)C3)cn2cc1NC(=O)c1cccc(C(F)F)n1] |
| 137 | 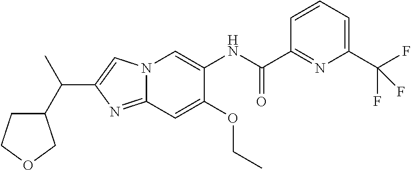 [SMILES: CCOc1cc2nc(C(C)C3CCOC3)cn2cc1NC(=O)c1cccc(C(F)(F)F)n1] |
| 138 | 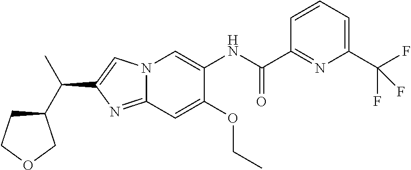 [SMILES: CCOc1cc2nc([C@H](C)[C@@H]3CCOC3)cn2cc1NC(=O)c1cccc(C(F)(F)F)n1] |
| 139 | 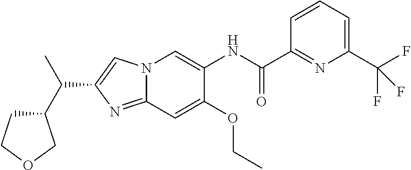 |
| 140 | 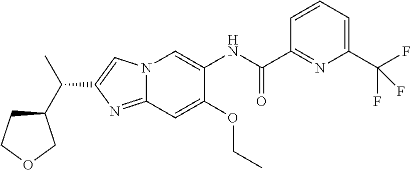 [SMILES: CCOc1cc2nc([C@@H](C)[C@@H]3CCOC3)cn2cc1NC(=O)c1cccc(C(F)(F)F)n1] |
| 141 | 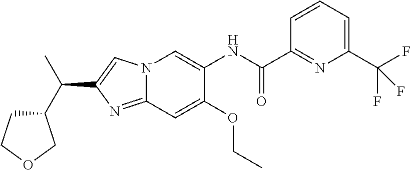 [SMILES: CCOc1cc2nc([C@H](C)[C@H]3CCOC3)cn2cc1NC(=O)c1cccc(C(F)(F)F)n1] |
| 142 | 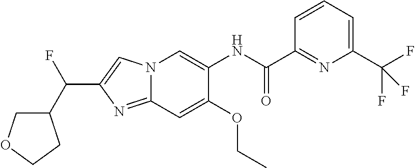 [SMILES: CCOc1cc2nc(C(F)C3CCOC3)cn2cc1NC(=O)c1cccc(C(F)(F)F)n1] |
| 143 | 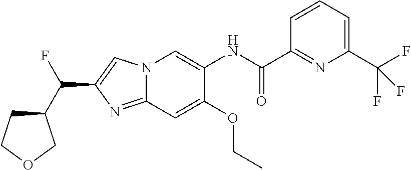 |
| 144 | 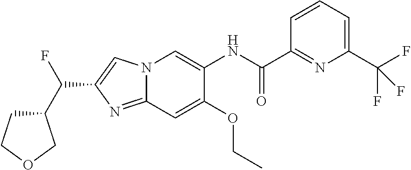 |
| 145 | 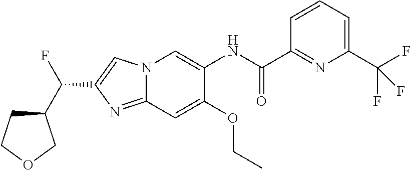 [SMILES: CCOc1cc2nc([C@@H](F)[C@@H]3CCOC3)cn2cc1NC(=O)c1cccc(C(F)(F)F)n1] |
| 146 | 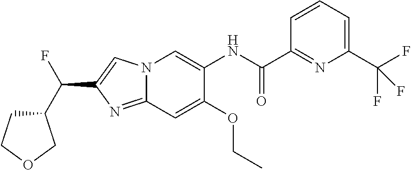 [SMILES: CCOc1cc2nc([C@H](F)[C@H]3CCOC3)cn2cc1NC(=O)c1cccc(C(F)(F)F)n1] |
| 147 | 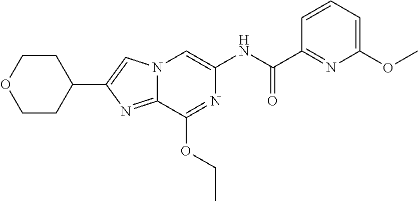 [SMILES: CCOc1nc(NC(=O)c2cccc(OC)n2)cn2cc(C3CCOCC3)nc12] |
| 148 | 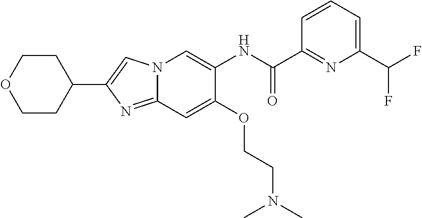 [SMILES: CN(C)CCOc1cc2nc(C3CCOCC3)cn2cc1NC(=O)c1cccc(C(F)F)n1] |
| 149 | 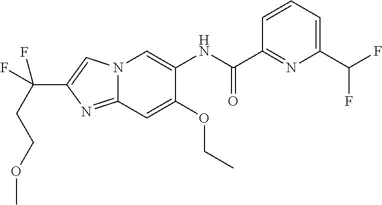 [SMILES: CCOc1cc2nc(C(F)(F)CCOC)cn2cc1NC(=O)c1cccc(C(F)F)n1] |
| 150 | 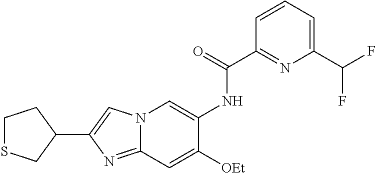 [SMILES: CCOc1cc2nc(C3CCSC3)cn2cc1NC(=O)c1cccc(C(F)F)n1] |
| 151 | 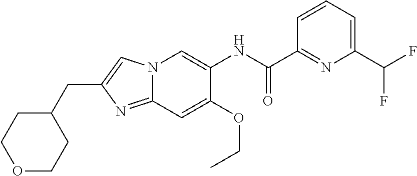 [SMILES: CCOc1cc2nc(CC3CCOCC3)cn2cc1NC(=O)c1cccc(C(F)F)n1] |
| 152 | 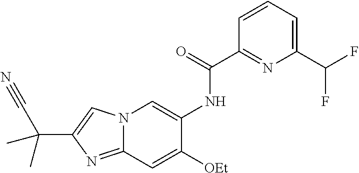 [SMILES: CCOc1cc2nc(C(C)(C)C#N)cn2cc1NC(=O)c1cccc(C(F)F)n1] |
| 153 | 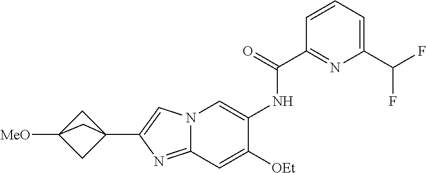 [SMILES: CCOc1cc2nc(C34CC(OC)(C3)C4)cn2cc1NC(=O)c1cccc(C(F)F)n1] |
| 154 | 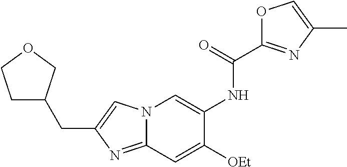 [SMILES: CCOc1cc2nc(CC3CCOC3)cn2cc1NC(=O)c1nc(C)co1] |
| 155 | 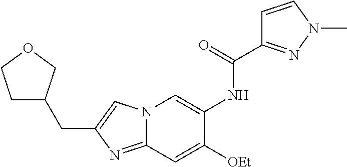 [SMILES: CCOc1cc2nc(CC3CCOC3)cn2cc1NC(=O)c1ccn(C)n1] |
| 156 | 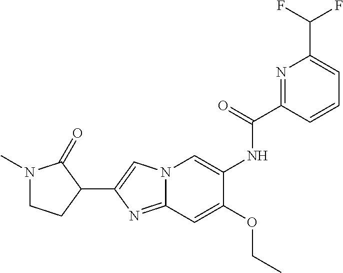 [SMILES: CCOc1cc2nc(C3CCN(C)C3=O)cn2cc1NC(=O)c1cccc(C(F)F)n1] |
| 157 | 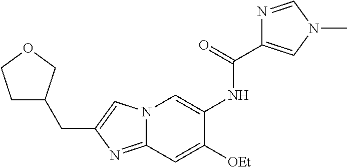 [SMILES: CCOc1cc2nc(CC3CCOC3)cn2cc1NC(=O)c1cn(C)cn1] |
| 158 | 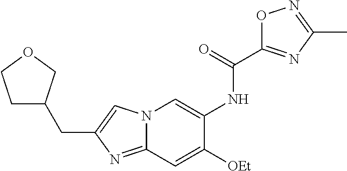 [SMILES: CCOc1cc2nc(CC3CCOC3)cn2cc1NC(=O)c1nc(C)no1] |
| 159 | 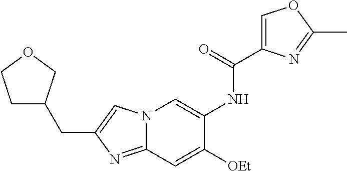 [SMILES: CCOc1cc2nc(CC3CCOC3)cn2cc1NC(=O)c1coc(C)n1] |
| 160 | 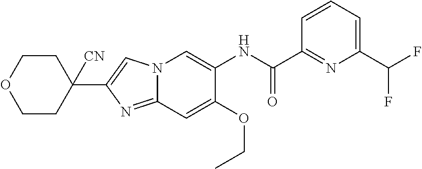 [SMILES: CCOc1cc2nc(C3(C#N)CCOCC3)cn2cc1NC(=O)c1cccc(C(F)F)n1] |
| 161 | 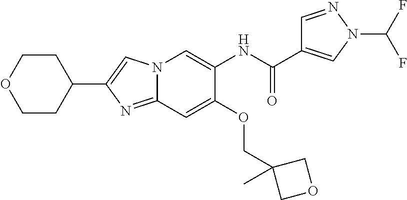 [SMILES: CC1(COc2cc3nc(C4CCOCC4)cn3cc2NC(=O)c2cnn(C(F)F)c2)COC1] |
| 162 | 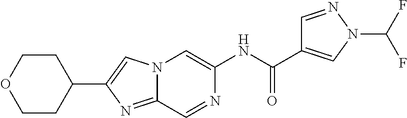 [SMILES: O=C(Nc1cn2cc(C3CCOCC3)nc2cn1)c1cnn(C(F)F)c1] |
| 163 | 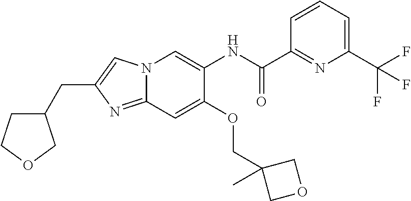 [SMILES: CC1(COc2cc3nc(CC4CCOC4)cn3cc2NC(=O)c2cccc(C(F)(F)F)n2)COC1] |
| 164 | 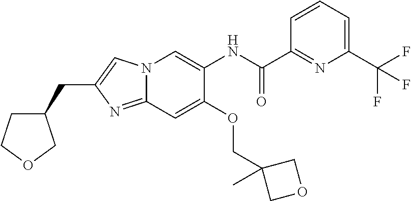 [SMILES: CC1(COc2cc3nc(C[C@@H]4CCOC4)cn3cc2NC(=O)c2cccc(C(F)(F)F)n2)COC1] |
| 165 | 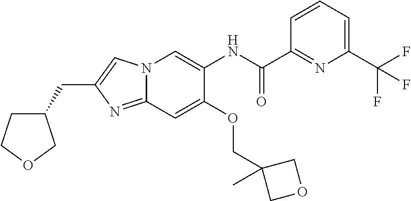 [SMILES: CC1(COc2cc3nc(C[C@H]4CCOC4)cn3cc2NC(=O)c2cccc(C(F)(F)F)n2)COC1] |
| 166 | 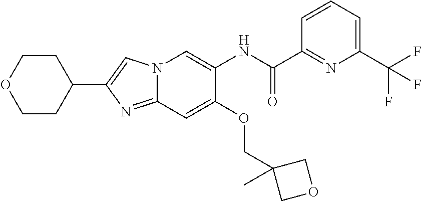 [SMILES: CC1(COc2cc3nc(C4CCOCC4)cn3cc2NC(=O)c2cccc(C(F)(F)F)n2)COC1] |
| 167 | 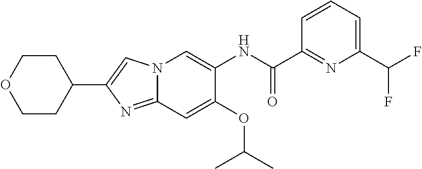 [SMILES: CC(C)Oc1cc2nc(C3CCOCC3)cn2cc1NC(=O)c1cccc(C(F)F)n1] |
| 168 | 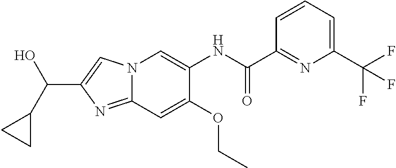 [SMILES: CCOc1cc2nc(C(O)C3CC3)cn2cc1NC(=O)c1cccc(C(F)(F)F)n1] |
| 169 | 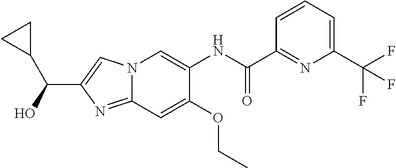 [SMILES: CCOc1cc2nc([C@@H](O)C3CC3)cn2cc1NC(=O)c1cccc(C(F)(F)F)n1] |
| 170 | 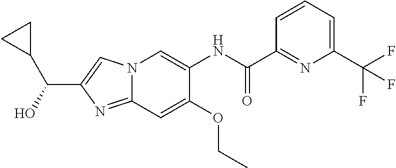 [SMILES: CCOc1cc2nc([C@H](O)C3CC3)cn2cc1NC(=O)c1cccc(C(F)(F)F)n1] |
| 171 | 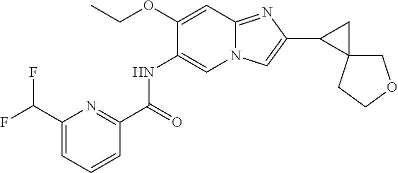 [SMILES: CCOc1cc2nc(C3CC34CCOC4)cn2cc1NC(=O)c1cccc(C(F)F)n1] |
| 172 | 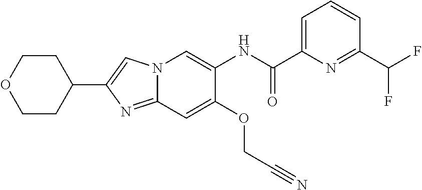 [SMILES: N#CCOc1cc2nc(C3CCOCC3)cn2cc1NC(=O)c1cccc(C(F)F)n1] |
| 173 | 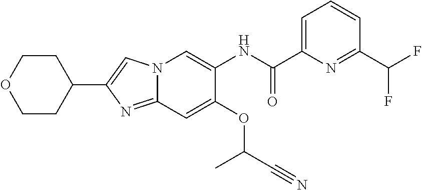 [SMILES: CC(C#N)Oc1cc2nc(C3CCOCC3)cn2cc1NC(=O)c1cccc(C(F)F)n1] |
| 174 | 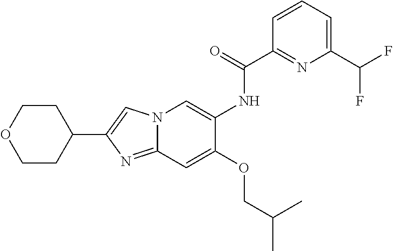 [SMILES: CC(C)COc1cc2nc(C3CCOCC3)cn2cc1NC(=O)c1cccc(C(F)F)n1] |
| 175 | 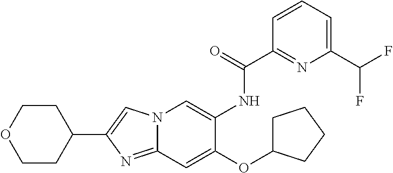 [SMILES: O=C(Nc1cn2cc(C3CCOCC3)nc2cc1OC1CCCC1)c1cccc(C(F)F)n1] |
| 176 | 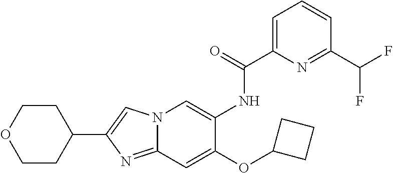 [SMILES: O=C(Nc1cn2cc(C3CCOCC3)nc2cc1OC1CCC1)c1cccc(C(F)F)n1] |
| 177 | 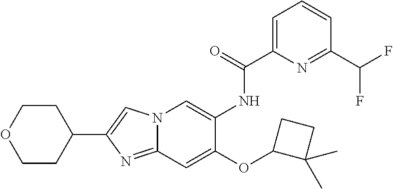 [SMILES: CC1(C)CCC1Oc1cc2nc(C3CCOCC3)cn2cc1NC(=O)c1cccc(C(F)F)n1] |
| 178 | 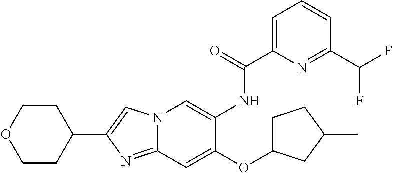 [SMILES: CC1CCC(Oc2cc3nc(C4CCOCC4)cn3cc2NC(=O)c2cccc(C(F)F)n2)C1] |
| 179 | 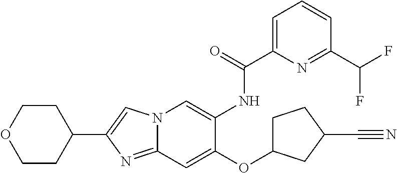 [SMILES: N#CC1CCC(Oc2cc3nc(C4CCOCC4)cn3cc2NC(=O)c2cccc(C(F)F)n2)C1] |
| 180 | 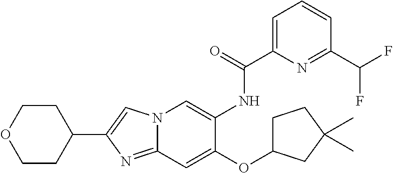 [SMILES: CC1(C)CCC(Oc2cc3nc(C4CCOCC4)cn3cc2NC(=O)c2cccc(C(F)F)n2)C1] |
| 181 | 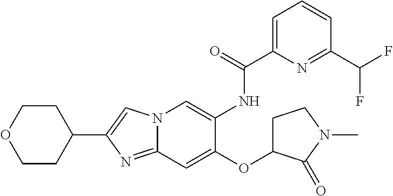 [SMILES: CN1CCC(Oc2cc3nc(C4CCOCC4)cn3cc2NC(=O)c2cccc(C(F)F)n2)C1=O] |
| 182 | 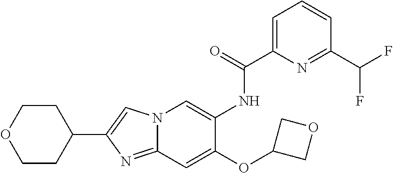 [SMILES: O=C(Nc1cn2cc(C3CCOCC3)nc2cc1OC1COC1)c1cccc(C(F)F)n1] |
| 183 | 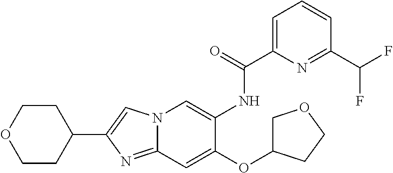 [SMILES: O=C(Nc1cn2cc(C3CCOCC3)nc2cc1OC1CCOC1)c1cccc(C(F)F)n1] |
| 184 | 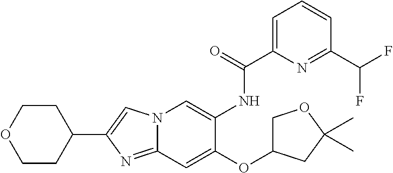 [SMILES: CC1(C)CC(Oc2cc3nc(C4CCOCC4)cn3cc2NC(=O)c2cccc(C(F)F)n2)CO1] |
| 185 | 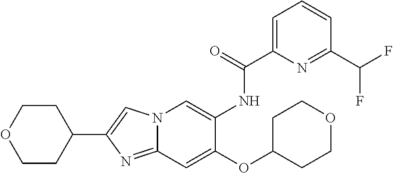 [SMILES: O=C(Nc1cn2cc(C3CCOCC3)nc2cc1OC1CCOCC1)c1cccc(C(F)F)n1] |
| 186 | 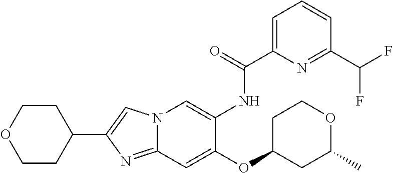 [SMILES: C[C@@H]1C[C@@H](Oc2cc3nc(C4CCOCC4)cn3cc2NC(=O)c2cccc(C(F)F)n2)CCO1] |
| 187 | 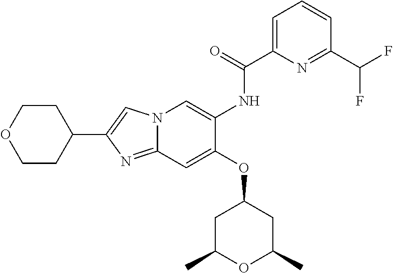 [SMILES: C[C@@H]1C[C@H](Oc2cc3nc(C4CCOCC4)cn3cc2NC(=O)c2cccc(C(F)F)n2)C[C@H](C)O1] |
| 188 | 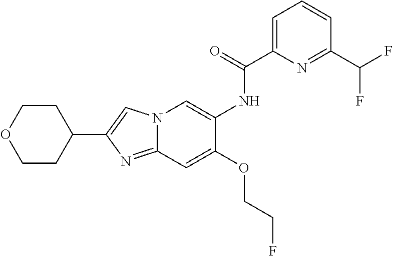 [SMILES: O=C(Nc1cn2cc(C3CCOCC3)nc2cc1OCCF)c1cccc(C(F)F)n1] |
| 189 | 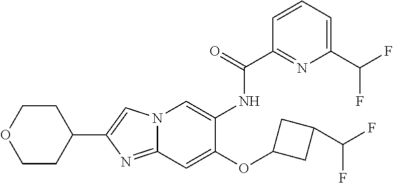 [SMILES: O=C(Nc1cn2cc(C3CCOCC3)nc2cc1OC1CC(C(F)F)C1)c1cccc(C(F)F)n1] |
| 190 | 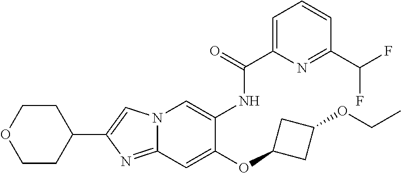 [SMILES: CCO[C@H]1C[C@H](Oc2cc3nc(C4CCOCC4)cn3cc2NC(=O)c2cccc(C(F)F)n2)C1] |
| 191 | 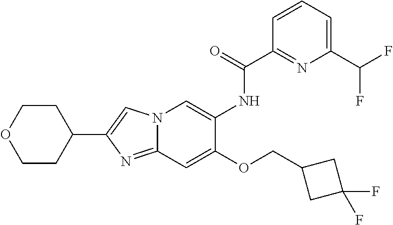 [SMILES: O=C(Nc1cn2cc(C3CCOCC3)nc2cc1OCC1CC(F)(F)C1)c1cccc(C(F)F)n1] |
| 192 | 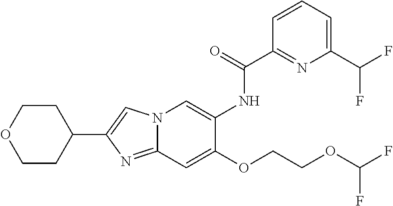 [SMILES: O=C(Nc1cn2cc(C3CCOCC3)nc2cc1OCCOC(F)F)c1cccc(C(F)F)n1] |
| 193 | 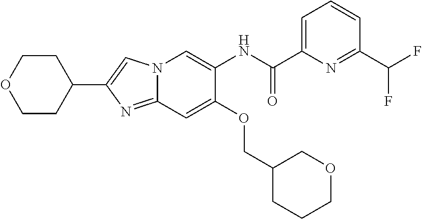 [SMILES: O=C(Nc1cn2cc(C3CCOCC3)nc2cc1OCC1CCCOC1)c1cccc(C(F)F)n1] |
| 194 | 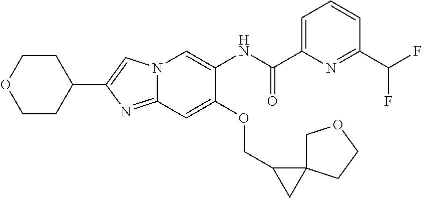 [SMILES: O=C(Nc1cn2cc(C3CCOCC3)nc2cc1OCC1CC12CCOC2)c1cccc(C(F)F)n1] |
| 195 | 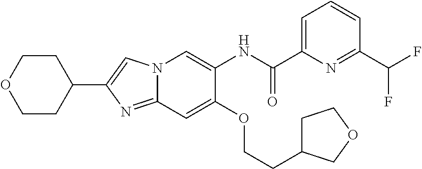 [SMILES: O=C(Nc1cn2cc(C3CCOCC3)nc2cc1OCCC1CCOC1)c1cccc(C(F)F)n1] |
| 196 | 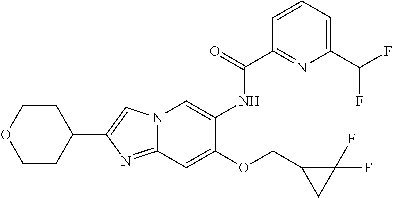 [SMILES: O=C(Nc1cn2cc(C3CCOCC3)nc2cc1OCC1CC1(F)F)c1cccc(C(F)F)n1] |
| 197 | 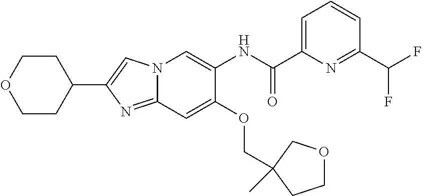 [SMILES: CC1(COc2cc3nc(C4CCOCC4)cn3cc2NC(=O)c2cccc(C(F)F)n2)CCOC1] |
| 198 | 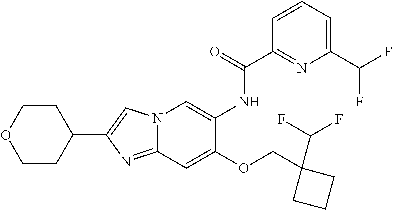 [SMILES: O=C(Nc1cn2cc(C3CCOCC3)nc2cc1OCC1(C(F)F)CCC1)c1cccc(C(F)F)n1] |
| 199 | 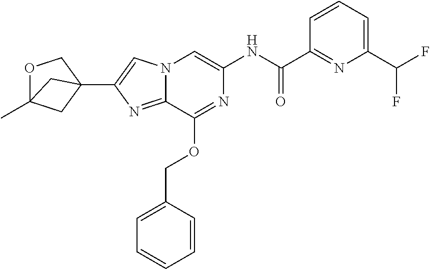 [SMILES: CC12CC(c3cn4cc(NC(=O)c5cccc(C(F)F)n5)nc(OCc5ccccc5)c4n3)(CO1)C2] |
| 200 | 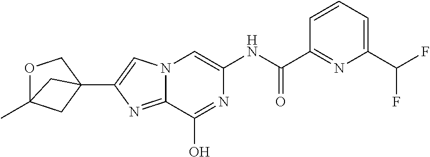 [SMILES: CC12CC(c3cn4cc(NC(=O)c5cccc(C(F)F)n5)nc(O)c4n3)(CO1)C2] |
| 201 | 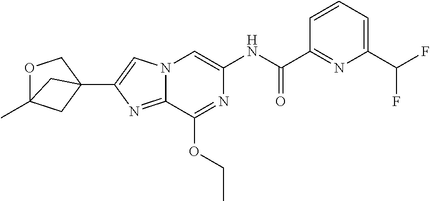 [SMILES: CCOc1nc(NC(=O)c2cccc(C(F)F)n2)cn2cc(C34COC(C)(C3)C4)nc12] |
| 202 | 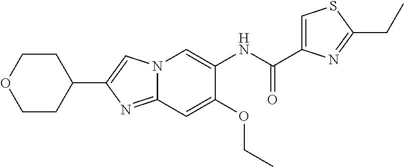 [SMILES: CCOc1cc2nc(C3CCOCC3)cn2cc1NC(=O)c1csc(CC)n1] |
| 203 | 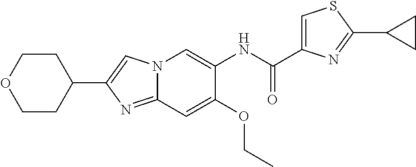 [SMILES: CCOc1cc2nc(C3CCOCC3)cn2cc1NC(=O)c1csc(C2CC2)n1] |
| 204 | 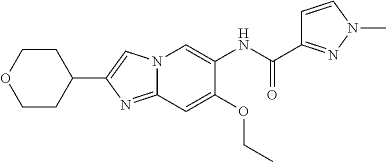 [SMILES: CCOc1cc2nc(C3CCOCC3)cn2cc1NC(=O)c1ccn(C)n1] |
| 205 | 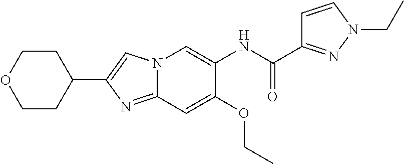 [SMILES: CCOc1cc2nc(C3CCOCC3)cn2cc1NC(=O)c1ccn(CC)n1] |
| 206 | 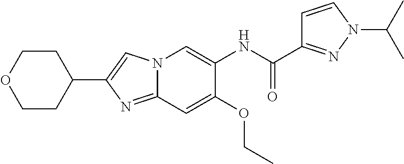 [SMILES: CCOc1cc2nc(C3CCOCC3)cn2cc1NC(=O)c1ccn(C(C)C)n1] |
| 207 | 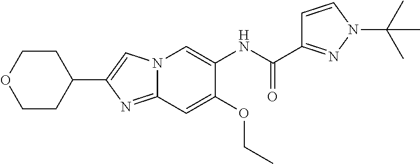 [SMILES: CCOc1cc2nc(C3CCOCC3)cn2cc1NC(=O)c1ccn(C(C)(C)C)n1] |
| 208 | 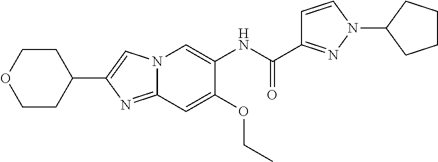 [SMILES: CCOc1cc2nc(C3CCOCC3)cn2cc1NC(=O)c1ccn(C2CCCC2)n1] |
| 209 | 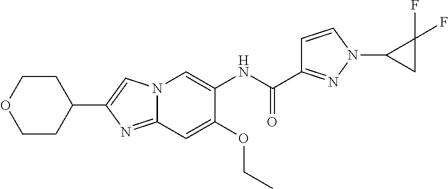 [SMILES: CCOc1cc2nc(C3CCOCC3)cn2cc1NC(=O)c1ccn(C2CC2(F)F)n1] |
| 210 | 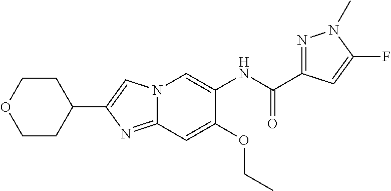 [SMILES: CCOc1cc2nc(C3CCOCC3)cn2cc1NC(=O)c1cc(F)n(C)n1] |
| 211 | 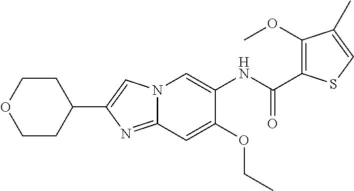 [SMILES: CCOc1cc2nc(C3CCOCC3)cn2cc1NC(=O)c1scc(C)c1OC] |
| 212 | 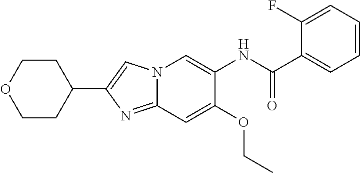 [SMILES: CCOc1cc2nc(C3CCOCC3)cn2cc1NC(=O)c1ccccc1F] |
| 213 | 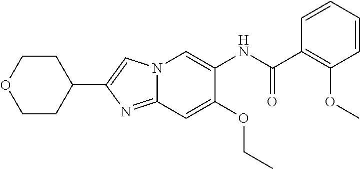 [SMILES: CCOc1cc2nc(C3CCOCC3)cn2cc1NC(=O)c1ccccc1OC] |
| 214 | 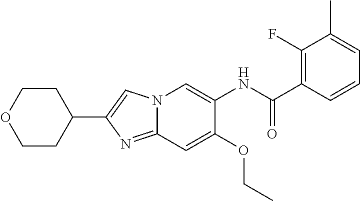 [SMILES: CCOc1cc2nc(C3CCOCC3)cn2cc1NC(=O)c1cccc(C)c1F] |
| 215 | 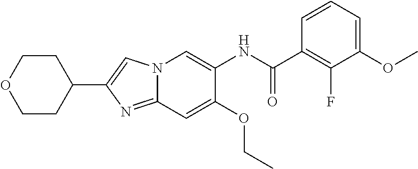 [SMILES: CCOc1cc2nc(C3CCOCC3)cn2cc1NC(=O)c1cccc(OC)c1F] |
| 216 | 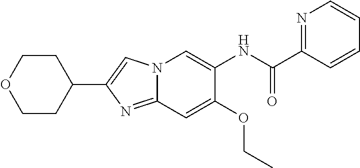 [SMILES: CCOc1cc2nc(C3CCOCC3)cn2cc1NC(=O)c1ccccn1] |
| 217 | 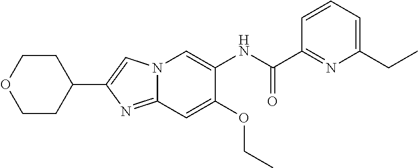 [SMILES: CCOc1cc2nc(C3CCOCC3)cn2cc1NC(=O)c1cccc(CC)n1] |
| 218 | 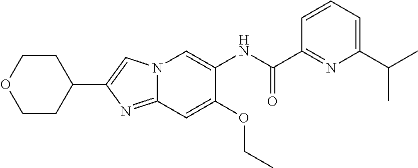 [SMILES: CCOc1cc2nc(C3CCOCC3)cn2cc1NC(=O)c1cccc(C(C)C)n1] |
| 219 | 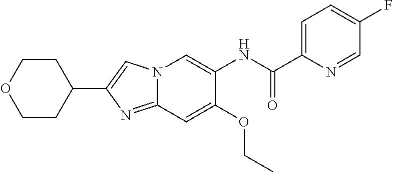 [SMILES: CCOc1cc2nc(C3CCOCC3)cn2cc1NC(=O)c1ccc(F)cn1] |
| 220 | 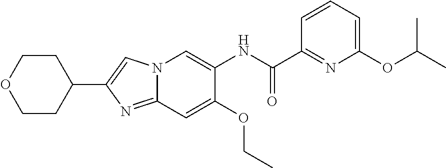 [SMILES: CCOc1cc2nc(C3CCOCC3)cn2cc1NC(=O)c1cccc(OC(C)C)n1] |
| 221 | 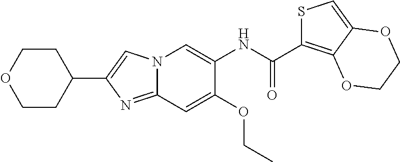 [SMILES: CCOc1cc2nc(C3CCOCC3)cn2cc1NC(=O)c1scc2c1OCCO2] |
| 222 | 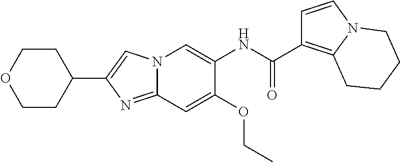 [SMILES: CCOc1cc2nc(C3CCOCC3)cn2cc1NC(=O)c1ccn2c1CCCC2] |
| 223 | 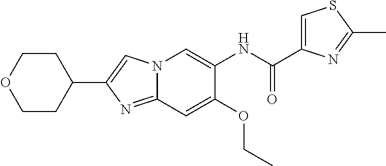 [SMILES: CCOc1cc2nc(C3CCOCC3)cn2cc1NC(=O)c1csc(C)n1] |
| 224 | 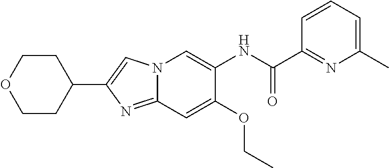 [SMILES: CCOc1cc2nc(C3CCOCC3)cn2cc1NC(=O)c1cccc(C)n1] |
| 225 | 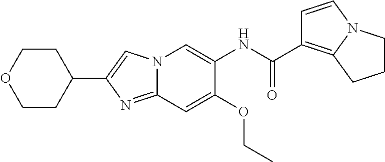 [SMILES: CCOc1cc2nc(C3CCOCC3)cn2cc1NC(=O)c1ccn2c1CCC2] |
| 226 | 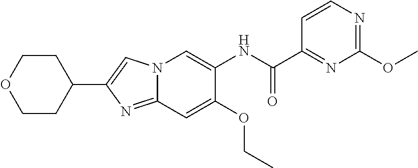 [SMILES: CCOc1cc2nc(C3CCOCC3)cn2cc1NC(=O)c1ccnc(OC)n1] |
| 227 | 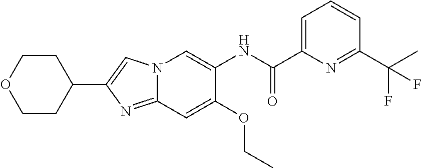 [SMILES: CCOc1cc2nc(C3CCOCC3)cn2cc1NC(=O)c1cccc(C(C)(F)F)n1] |
| 228 | 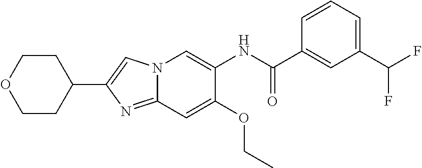 [SMILES: CCOc1cc2nc(C3CCOCC3)cn2cc1NC(=O)c1cccc(C(F)F)c1] |
| 229 | 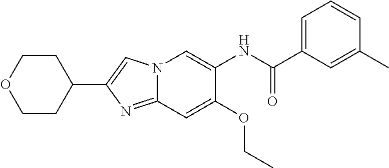 [SMILES: CCOc1cc2nc(C3CCOCC3)cn2cc1NC(=O)c1cccc(C)c1] |
| 230 | 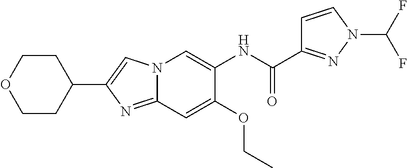 [SMILES: CCOc1cc2nc(C3CCOCC3)cn2cc1NC(=O)c1ccn(C(F)F)n1] |
| 231 | 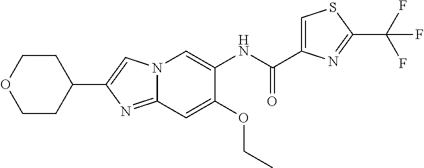 [SMILES: CCOc1cc2nc(C3CCOCC3)cn2cc1NC(=O)c1csc(C(F)(F)F)n1] |
| 232 | 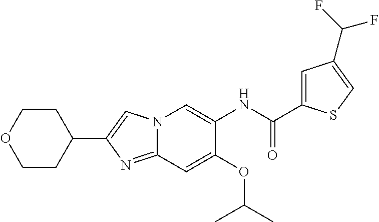 [SMILES: CC(C)Oc1cc2nc(C3CCOCC3)cn2cc1NC(=O)c1cc(C(F)F)cs1] |
| 233 | 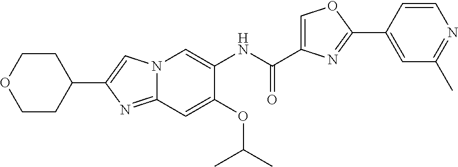 [SMILES: Cc1cc(-c2nc(C(=O)Nc3cn4cc(C5CCOCC5)nc4cc3OC(C)C)co2)ccn1] |
| 234 | 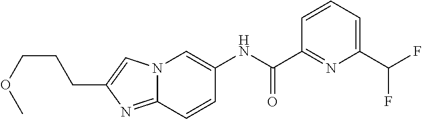 [SMILES: COCCCc1cn2cc(NC(=O)c3cccc(C(F)F)n3)ccc2n1] |
| 235 | 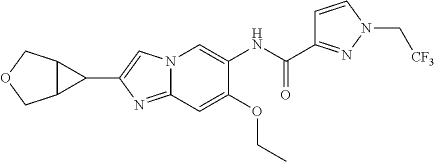 [SMILES: CCOc1cc2nc(C3C4COCC43)cn2cc1NC(=O)c1ccn(CC(F)(F)F)n1] |
| 236 | 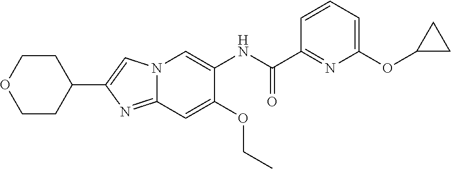 [SMILES: CCOc1cc2nc(C3CCOCC3)cn2cc1NC(=O)c1cccc(OC2CC2)n1] |
| 237 | 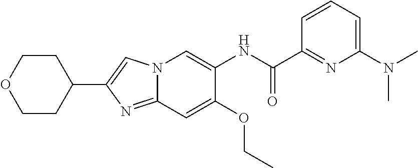 [SMILES: CCOc1cc2nc(C3CCOCC3)cn2cc1NC(=O)c1cccc(N(C)C)n1] |
| 238 | 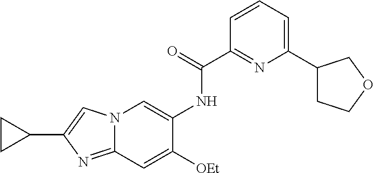 [SMILES: CCOc1cc2nc(C3CC3)cn2cc1NC(=O)c1cccc(C2CCOC2)n1] |
| 239 | 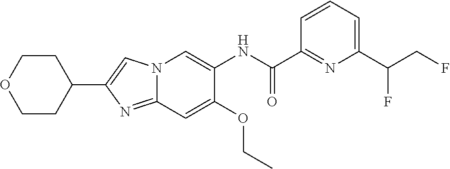 [SMILES: CCOc1cc2nc(C3CCOCC3)cn2cc1NC(=O)c1cccc(C(F)CF)n1] |
| 240 | 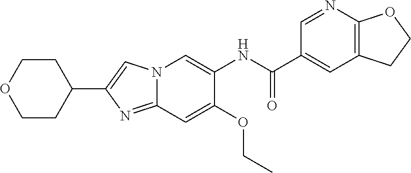 [SMILES: CCOc1cc2nc(C3CCOCC3)cn2cc1NC(=O)c1cnc2c(c1)CCO2] |
| 241 | 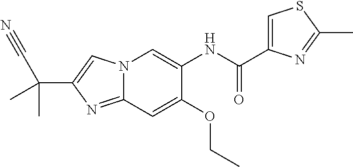 [SMILES: CCOc1cc2nc(C(C)(C)C#N)cn2cc1NC(=O)c1csc(C)n1] |
| 242 | 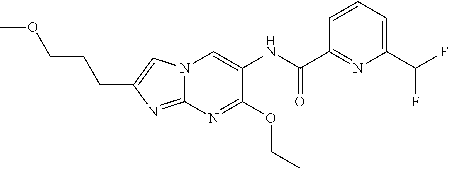 [SMILES: CCOc1nc2nc(CCCOC)cn2cc1NC(=O)c1cccc(C(F)F)n1] |
| 243 | 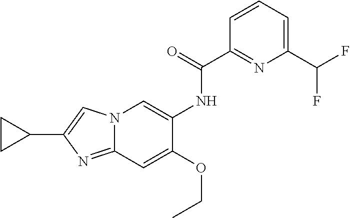 [SMILES: CCOc1cc2nc(C3CC3)cn2cc1NC(=O)c1cccc(C(F)F)n1] |
| 244 | 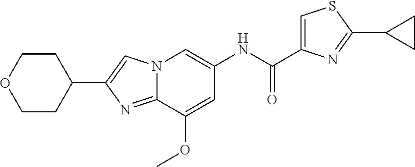 [SMILES: COc1cc(NC(=O)c2csc(C3CC3)n2)cn2cc(C3CCOCC3)nc12] |
| 245 | 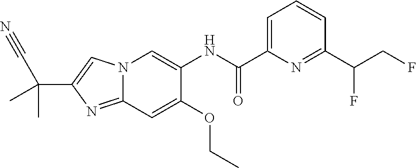 [SMILES: CCOc1cc2nc(C(C)(C)C#N)cn2cc1NC(=O)c1cccc(C(F)CF)n1] |
| 246 | 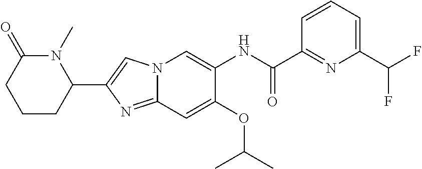 [SMILES: CC(C)Oc1cc2nc(C3CCCC(=O)N3C)cn2cc1NC(=O)c1cccc(C(F)F)n1] |
| 247 | 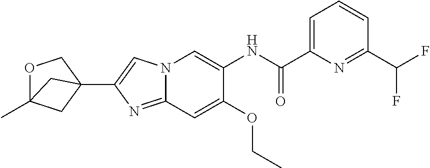 [SMILES: CCOc1cc2nc(C34COC(C)(C3)C4)cn2cc1NC(=O)c1cccc(C(F)F)n1] |
| 248 | 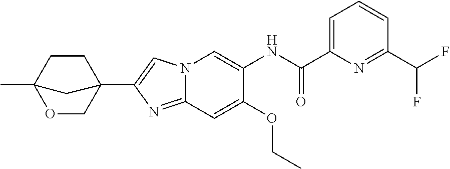 [SMILES: CCOc1cc2nc(C34CCC(C)(C3)OC4)cn2cc1NC(=O)c1cccc(C(F)F)n1] |
| 249 | 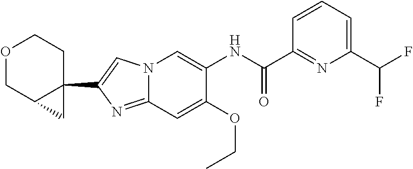 [SMILES: CCOc1cc2nc([C@]34CCOC[C@H]3C4)cn2cc1NC(=O)c1cccc(C(F)F)n1] |
| 250 | 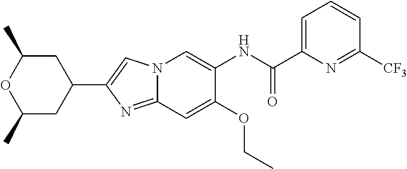 [SMILES: CCOc1cc2nc(C3C[C@@H](C)O[C@@H](C)C3)cn2cc1NC(=O)c1cccc(C(F)(F)F)n1] |
| 251 | 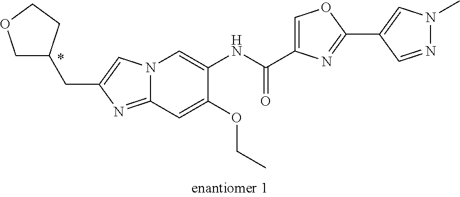 [SMILES: CCOc1cc2nc(CC3CCOC3)cn2cc1NC(=O)c1coc(-c2cnn(C)c2)n1] |
| 252 | 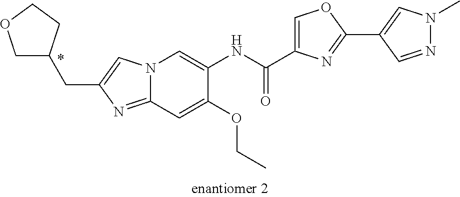 [SMILES: CCOc1cc2nc(CC3CCOC3)cn2cc1NC(=O)c1coc(-c2cnn(C)c2)n1] |
| 253 | 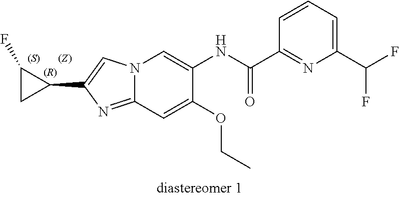 |
| 254 | 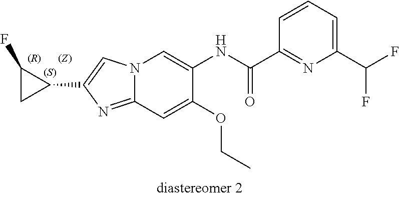 |
| 255 | 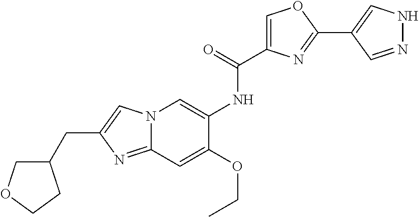 [SMILES: CCOc1cc2nc(CC3CCOC3)cn2cc1NC(=O)c1coc(-c2cn[nH]c2)n1] |
| 256 | 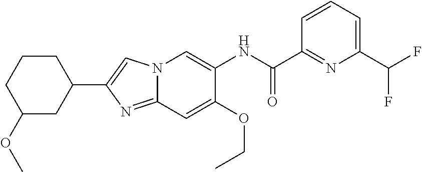 [SMILES: CCOc1cc2nc(C3CCCC(OC)C3)cn2cc1NC(=O)c1cccc(C(F)F)n1] |
| 257 | 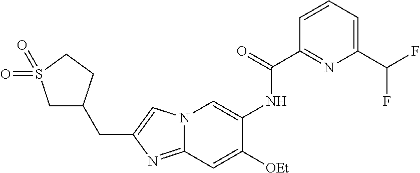 [SMILES: CCOc1cc2nc(CC3CCS(=O)(=O)C3)cn2cc1NC(=O)c1cccc(C(F)F)n1] |
| 258 | 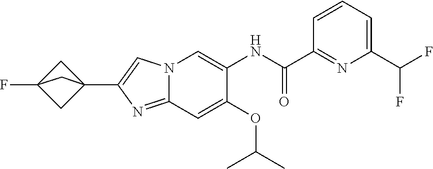 [SMILES: CC(C)Oc1cc2nc(C34CC(F)(C3)C4)cn2cc1NC(=O)c1cccc(C(F)F)n1] |
| 259 | 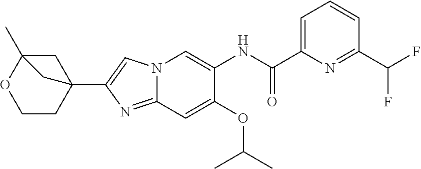 [SMILES: CC(C)Oc1cc2nc(C34CCOC(C)(C3)C4)cn2cc1NC(=O)c1cccc(C(F)F)n1] |
| 260 | 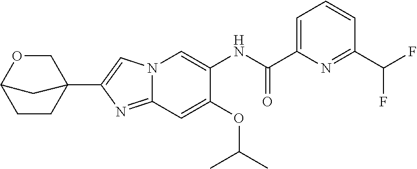 [SMILES: CC(C)Oc1cc2nc(C34CCC(C3)OC4)cn2cc1NC(=O)c1cccc(C(F)F)n1] |
| 261 | 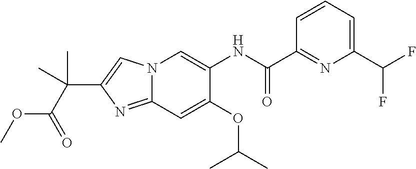 [SMILES: COC(=O)C(C)(C)c1cn2cc(NC(=O)c3cccc(C(F)F)n3)c(OC(C)C)cc2n1] |
| 262 | 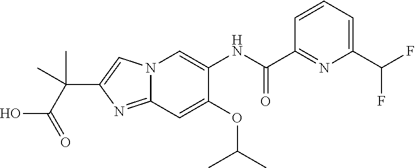 [SMILES: CC(C)Oc1cc2nc(C(C)(C)C(=O)O)cn2cc1NC(=O)c1cccc(C(F)F)n1] |
| 263 | 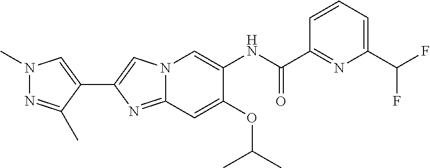 [SMILES: Cc1nn(C)cc1-c1cn2cc(NC(=O)c3cccc(C(F)F)n3)c(OC(C)C)cc2n1] |
| 264 | 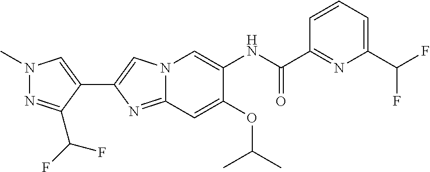 [SMILES: CC(C)Oc1cc2nc(-c3cn(C)nc3C(F)F)cn2cc1NC(=O)c1cccc(C(F)F)n1] |
| 265 | 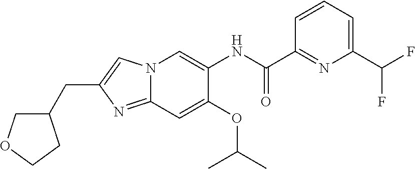 [SMILES: CC(C)Oc1cc2nc(CC3CCOC3)cn2cc1NC(=O)c1cccc(C(F)F)n1] |
| 266 | 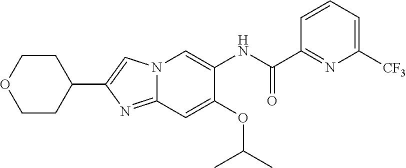 [SMILES: CC(C)Oc1cc2nc(C3CCOCC3)cn2cc1NC(=O)c1cccc(C(F)(F)F)n1] |
| 267 | 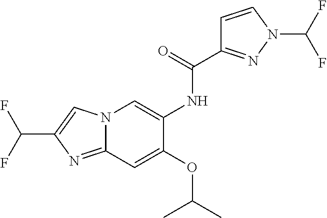 [SMILES: CC(C)Oc1cc2nc(C(F)F)cn2cc1NC(=O)c1ccn(C(F)F)n1] |
| 268 | 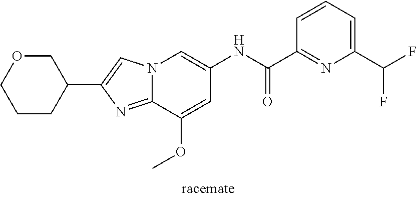 [SMILES: COc1cc(NC(=O)c2cccc(C(F)F)n2)cn2cc(C3CCCOC3)nc12] |
| 269 | 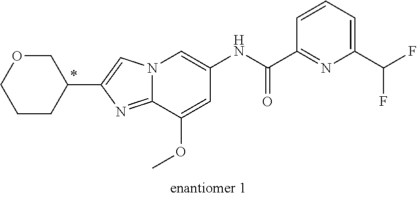 [SMILES: COc1cc(NC(=O)c2cccc(C(F)F)n2)cn2cc(C3CCCOC3)nc12] |
| 270 | 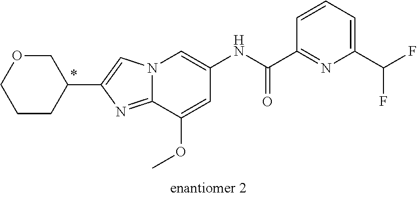 [SMILES: COc1cc(NC(=O)c2cccc(C(F)F)n2)cn2cc(C3CCCOC3)nc12] |
| 271 | 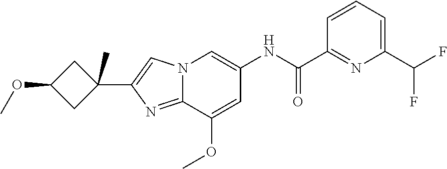 [SMILES: COc1cc(NC(=O)c2cccc(C(F)F)n2)cn2cc([C@]3(C)C[C@@H](OC)C3)nc12] |
| 272 | 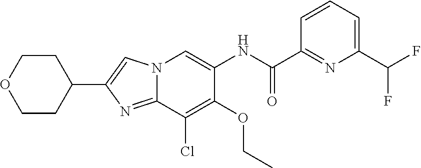 [SMILES: CCOc1c(NC(=O)c2cccc(C(F)F)n2)cn2cc(C3CCOCC3)nc2c1Cl] |
| 273 | 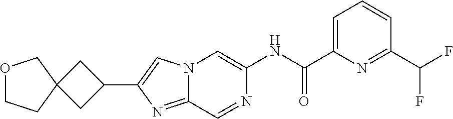 [SMILES: O=C(Nc1cn2cc(C3CC4(CCOC4)C3)nc2cn1)c1cccc(C(F)F)n1] |
| 274 | 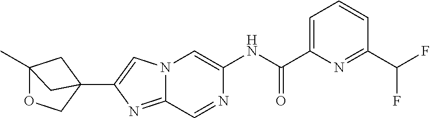 [SMILES: CC12CC(c3cn4cc(NC(=O)c5cccc(C(F)F)n5)ncc4n3)(CO1)C2] |
| 275 | 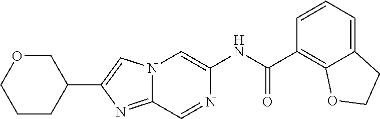 [SMILES: O=C(Nc1cn2cc(C3CCCOC3)nc2cn1)c1cccc2c1OCC2] |
| 276 | 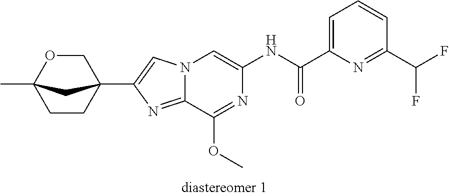 [SMILES: COc1nc(NC(=O)c2cccc(C(F)F)n2)cn2cc([C@@]34CC[C@@](C)(C3)OC4)nc12] |
| 277 | 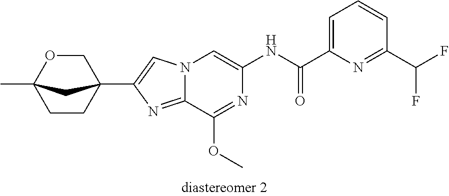 [SMILES: COc1nc(NC(=O)c2cccc(C(F)F)n2)cn2cc([C@@]34CC[C@@](C)(C3)OC4)nc12] |
| 278 | 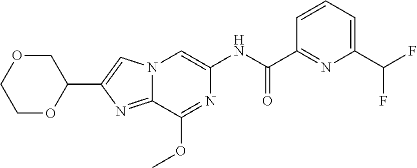 [SMILES: COc1nc(NC(=O)c2cccc(C(F)F)n2)cn2cc(C3COCCO3)nc12] |
| 279 | 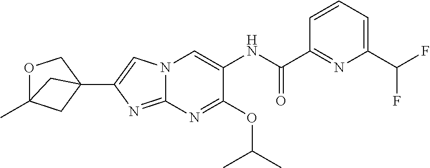 [SMILES: CC(C)Oc1nc2nc(C34COC(C)(C3)C4)cn2cc1NC(=O)c1cccc(C(F)F)n1] |
| 280 | 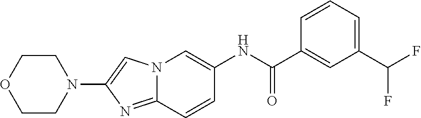 [SMILES: O=C(Nc1ccc2nc(N3CCOCC3)cn2c1)c1cccc(C(F)F)c1] |
| 281 | 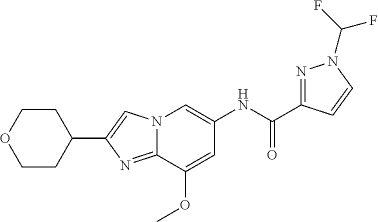 [SMILES: COc1cc(NC(=O)c2ccn(C(F)F)n2)cn2cc(C3CCOCC3)nc12] |
| 282 | 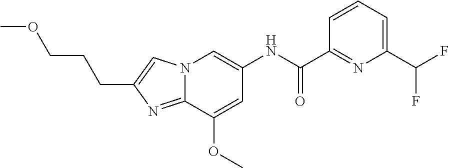 [SMILES: COCCCc1cn2cc(NC(=O)c3cccc(C(F)F)n3)cc(OC)c2n1] |
| 283 | 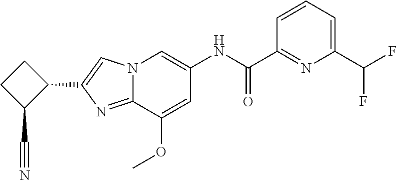 [SMILES: COc1cc(NC(=O)c2cccc(C(F)F)n2)cn2cc([C@H]3CC[C@@H]3C#N)nc12] |
| 284 | 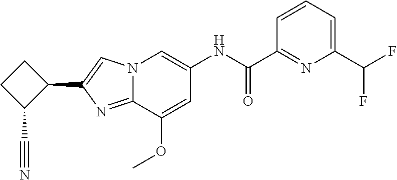 [SMILES: COc1cc(NC(=O)c2cccc(C(F)F)n2)cn2cc([C@@H]3CC[C@H]3C#N)nc12] |
| 285 | 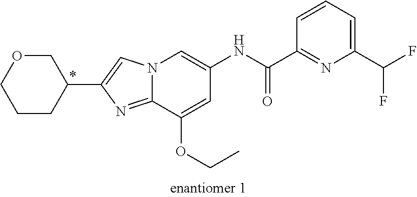 [SMILES: CCOc1cc(NC(=O)c2cccc(C(F)F)n2)cn2cc(C3CCCOC3)nc12] |
| 286 | 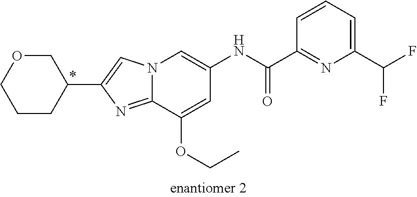 [SMILES: CCOc1cc(NC(=O)c2cccc(C(F)F)n2)cn2cc(C3CCCOC3)nc12] |
| 287 | 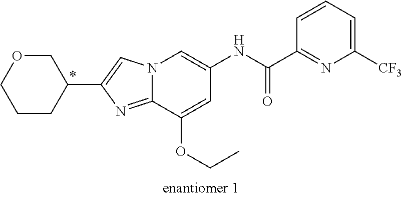 [SMILES: CCOc1cc(NC(=O)c2cccc(C(F)(F)F)n2)cn2cc(C3CCCOC3)nc12] |
| 288 | 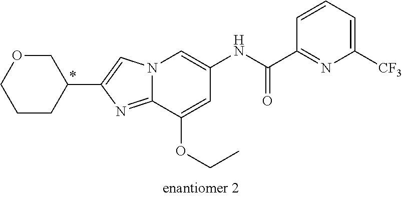 [SMILES: CCOc1cc(NC(=O)c2cccc(C(F)(F)F)n2)cn2cc(C3CCCOC3)nc12] |
| 289 | 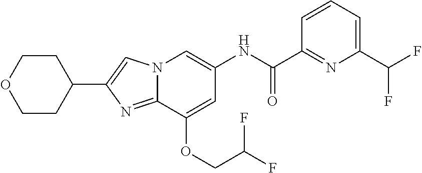 [SMILES: O=C(Nc1cc(OCC(F)F)c2nc(C3CCOCC3)cn2c1)c1cccc(C(F)F)n1] |
| 290 | 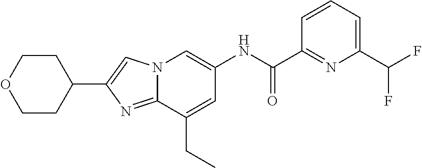 [SMILES: CCc1cc(NC(=O)c2cccc(C(F)F)n2)cn2cc(C3CCOCC3)nc12] |
| 291 | 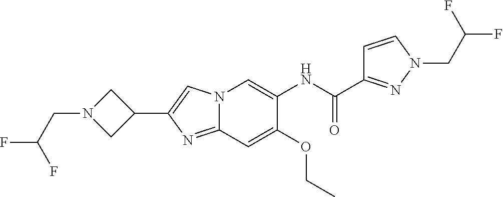 [SMILES: CCOc1cc2nc(C3CN(CC(F)F)C3)cn2cc1NC(=O)c1ccn(CC(F)F)n1] |
| 292 | 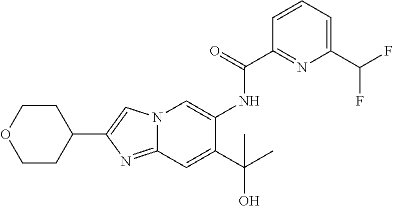 [SMILES: CC(C)(O)c1cc2nc(C3CCOCC3)cn2cc1NC(=O)c1cccc(C(F)F)n1] |
| 293 | 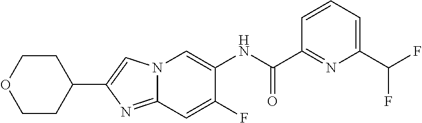 [SMILES: O=C(Nc1cn2cc(C3CCOCC3)nc2cc1F)c1cccc(C(F)F)n1] |
| 294 | 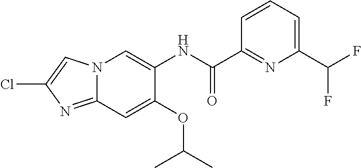 [SMILES: CC(C)Oc1cc2nc(Cl)cn2cc1NC(=O)c1cccc(C(F)F)n1] |
| 295 | 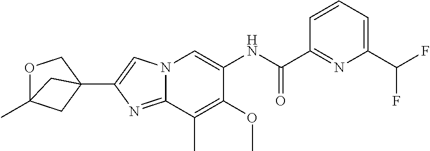 [SMILES: COc1c(NC(=O)c2cccc(C(F)F)n2)cn2cc(C34COC(C)(C3)C4)nc2c1C] |
| 296 | 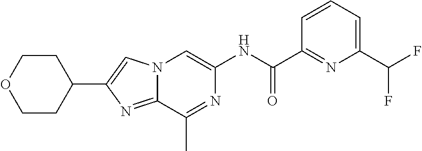 [SMILES: Cc1nc(NC(=O)c2cccc(C(F)F)n2)cn2cc(C3CCOCC3)nc12] |
| 297 | 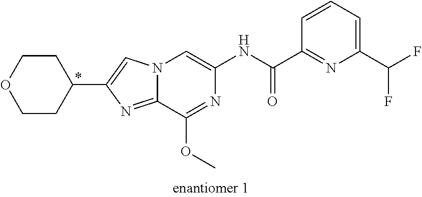 [SMILES: COc1nc(NC(=O)c2cccc(C(F)F)n2)cn2cc(C3CCOCC3)nc12] |
| 298 | 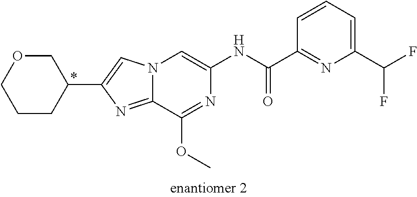 [SMILES: COc1nc(NC(=O)c2cccc(C(F)F)n2)cn2cc(C3CCCOC3)nc12] |
| 299 | 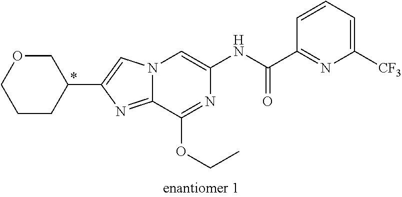 [SMILES: CCOc1nc(NC(=O)c2cccc(C(F)(F)F)n2)cn2cc(C3CCCOC3)nc12] |
| 300 | 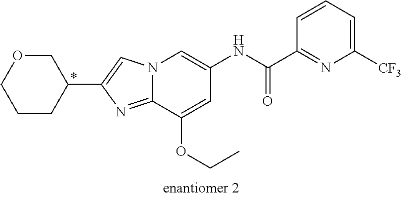 [SMILES: CCOc1cc(NC(=O)c2cccc(C(F)(F)F)n2)cn2cc(C3CCCOC3)nc12] |
| 301 | 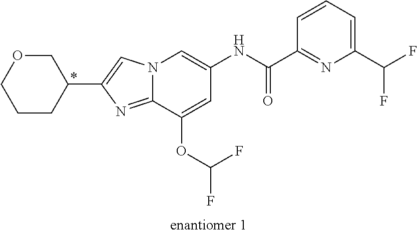 [SMILES: O=C(Nc1cc(OC(F)F)c2nc(C3CCCOC3)cn2c1)c1cccc(C(F)F)n1] |
| 302 | 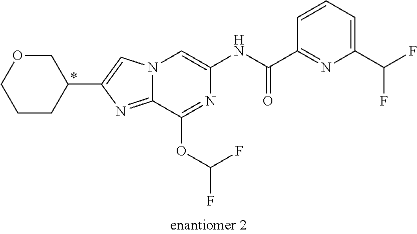 [SMILES: O=C(Nc1cn2cc(C3CCCOC3)nc2c(OC(F)F)n1)c1cccc(C(F)F)n1] |
| 303 | 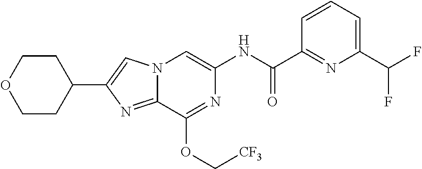 [SMILES: O=C(Nc1cn2cc(C3CCOCC3)nc2c(OCC(F)(F)F)n1)c1cccc(C(F)F)n1] |
| 304 | 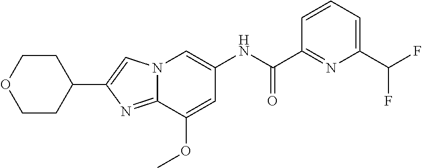 [SMILES: COc1cc(NC(=O)c2cccc(C(F)F)n2)cn2cc(C3CCOCC3)nc12] |
| 305 | 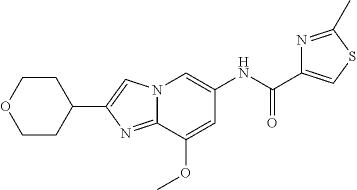 [SMILES: COc1cc(NC(=O)c2csc(C)n2)cn2cc(C3CCOCC3)nc12] |
| 306 | 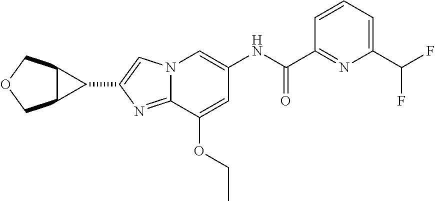 |
| 307 | 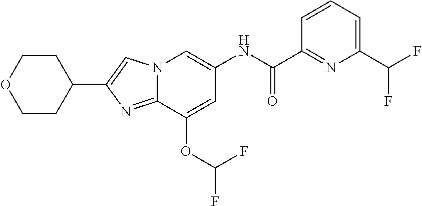 [SMILES: O=C(Nc1cc(OC(F)F)c2nc(C3CCOCC3)cn2c1)c1cccc(C(F)F)n1] |
| 308 | 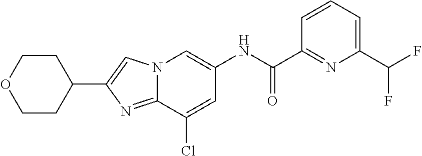 [SMILES: O=C(Nc1cc(Cl)c2nc(C3CCOCC3)cn2c1)c1cccc(C(F)F)n1] |
| 309 | 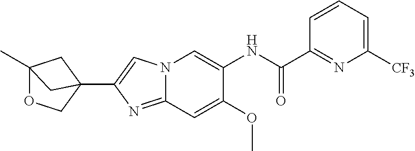 [SMILES: COc1cc2nc(C34COC(C)(C3)C4)cn2cc1NC(=O)c1cccc(C(F)(F)F)n1] |
| 310 | 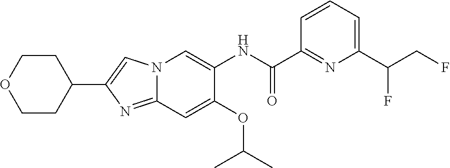 [SMILES: CC(C)Oc1cc2nc(C3CCOCC3)cn2cc1NC(=O)c1cccc(C(F)CF)n1] |
| 311 | 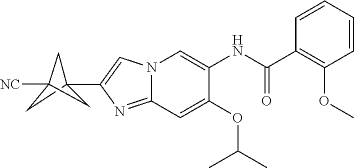 [SMILES: COc1ccccc1C(=O)Nc1cn2cc(C34CC(C#N)(C3)C4)nc2cc1OC(C)C] |
| 312 | 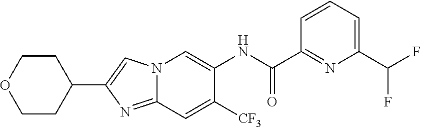 [SMILES: O=C(Nc1cn2cc(C3CCOCC3)nc2cc1C(F)(F)F)c1cccc(C(F)F)n1] |
| 313 | 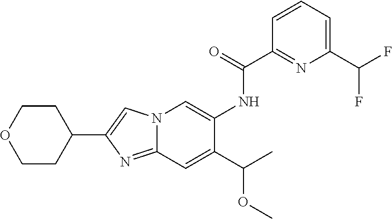 [SMILES: COC(C)c1cc2nc(C3CCOCC3)cn2cc1NC(=O)c1cccc(C(F)F)n1] |
| 314 | 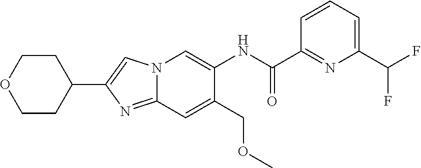 [SMILES: COCc1cc2nc(C3CCOCC3)cn2cc1NC(=O)c1cccc(C(F)F)n1] |
| 315 | 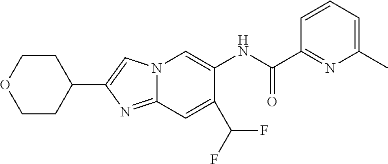 [SMILES: Cc1cccc(C(=O)Nc2cn3cc(C4CCOCC4)nc3cc2C(F)F)n1] |
| 316 | 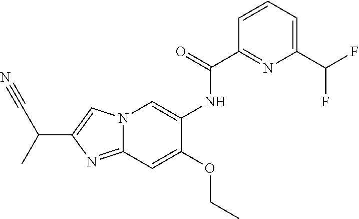 [SMILES: CCOc1cc2nc(C(C)C#N)cn2cc1NC(=O)c1cccc(C(F)F)n1] |
| 317 | 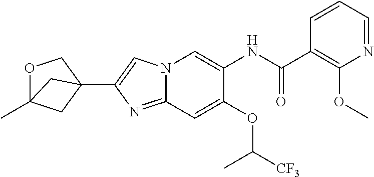 |
| 318 | 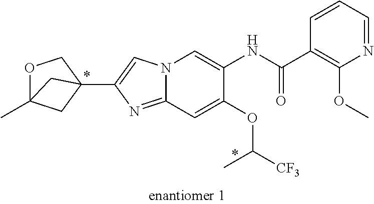 |
| 319 | 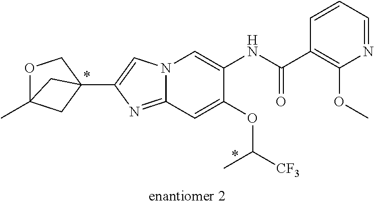 |
| 320 | 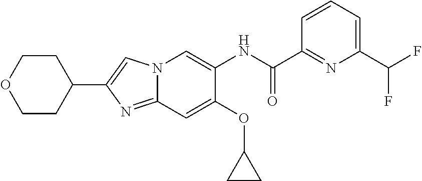 [SMILES: O=C(Nc1cn2cc(C3CCOCC3)nc2cc1OC1CC1)c1cccc(C(F)F)n1] |
| 321 | 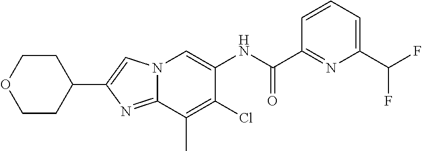 [SMILES: Cc1c(Cl)c(NC(=O)c2cccc(C(F)F)n2)cn2cc(C3CCOCC3)nc12] |
| 322 | 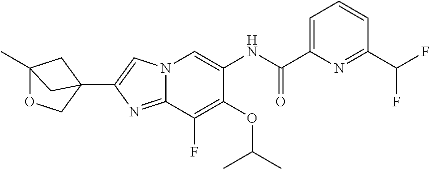 [SMILES: CC(C)Oc1c(NC(=O)c2cccc(C(F)F)n2)cn2cc(C34COC(C)(C3)C4)nc2c1F] |
| 323 | 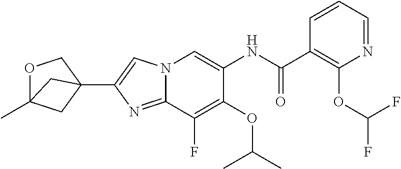 [SMILES: CC(C)Oc1c(NC(=O)c2cccnc2OC(F)F)cn2cc(C34COC(C)(C3)C4)nc2c1F] |
| 324 | 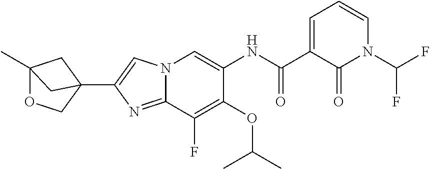 [SMILES: CC(C)Oc1c(NC(=O)c2cccn(C(F)F)c2=O)cn2cc(C34COC(C)(C3)C4)nc2c1F] |
| 325 | 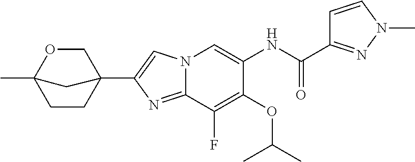 |
| 326 | 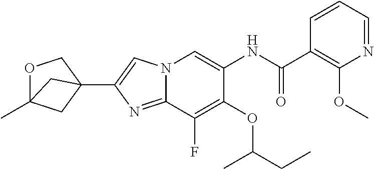 |
| 327 | 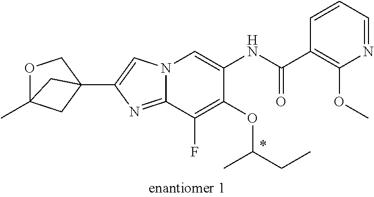 |
| 328 | 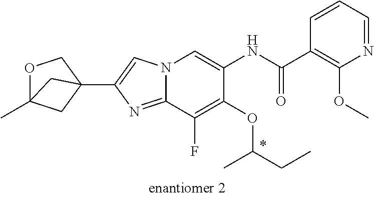 |
| 329 | 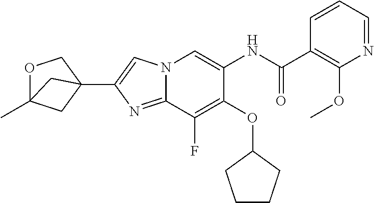 [SMILES: COc1ncccc1C(=O)Nc1cn2cc(C34COC(C)(C3)C4)nc2c(F)c1OC1CCCC1] |
| 330 | 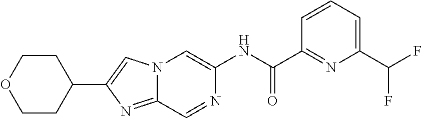 [SMILES: O=C(Nc1cn2cc(C3CCOCC3)nc2cn1)c1cccc(C(F)F)n1] |
| 331 | 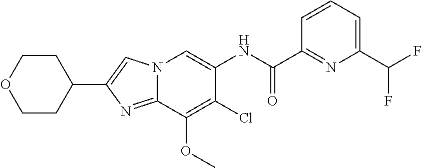 [SMILES: COc1c(Cl)c(NC(=O)c2cccc(C(F)F)n2)cn2cc(C3CCOCC3)nc12] |
| 332 | 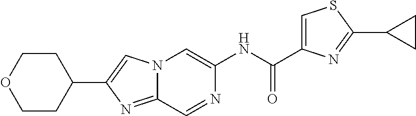 [SMILES: O=C(Nc1cn2cc(C3CCOCC3)nc2cn1)c1csc(C2CC2)n1] |
| 333 | 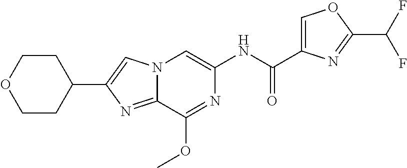 [SMILES: COc1nc(NC(=O)c2coc(C(F)F)n2)cn2cc(C3CCOCC3)nc12] |
| 334 | 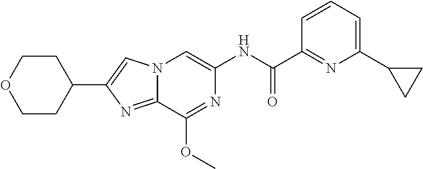 [SMILES: COc1nc(NC(=O)c2cccc(C3CC3)n2)cn2cc(C3CCOCC3)nc12] |
| 335 | 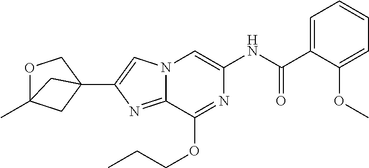 |
| 336 | 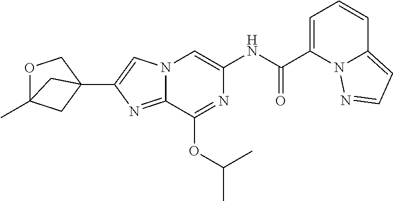 [SMILES: CC(C)Oc1nc(NC(=O)c2cccc3ccnn23)cn2cc(C34COC(C)(C3)C4)nc12] |
| 337 | 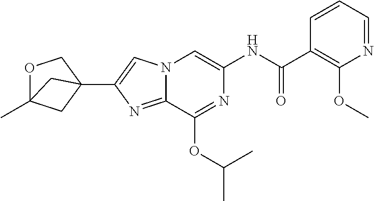 |
| 338 | 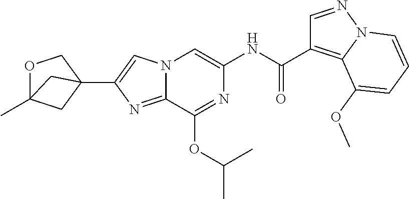 [SMILES: COc1cccn2ncc(C(=O)Nc3cn4cc(C56COC(C)(C5)C6)nc4c(OC(C)C)n3)c12] |
| 339 | 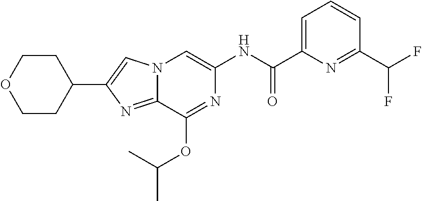 [SMILES: CC(C)Oc1nc(NC(=O)c2cccc(C(F)F)n2)cn2cc(C3CCOCC3)nc12] |
| 340 | 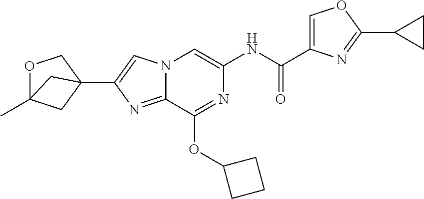 [SMILES: CC12CC(c3cn4cc(NC(=O)c5coc(C6CC6)n5)nc(OC5CCC5)c4n3)(CO1)C2] |
| 341 | 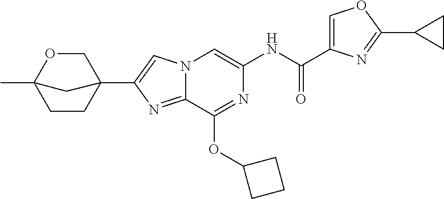 [SMILES: CC12CCC(c3cn4cc(NC(=O)c5coc(C6CC6)n5)nc(OC5CCC5)c4n3)(CO1)C2] |
| 342 | 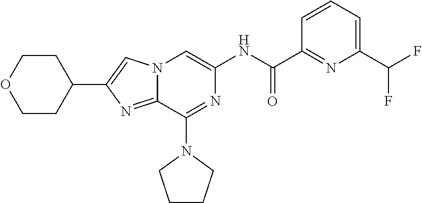 [SMILES: O=C(Nc1cn2cc(C3CCOCC3)nc2c(N2CCCC2)n1)c1cccc(C(F)F)n1] |
| 343 | 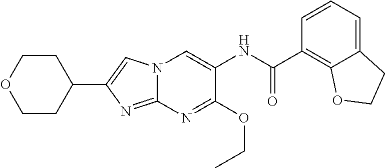 [SMILES: CCOc1nc2nc(C3CCOCC3)cn2cc1NC(=O)c1cccc2c1OCC2] |
| 344 | 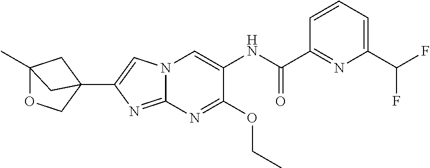 [SMILES: CCOc1nc2nc(C34COC(C)(C3)C4)cn2cc1NC(=O)c1cccc(C(F)F)n1] |
| 345 | 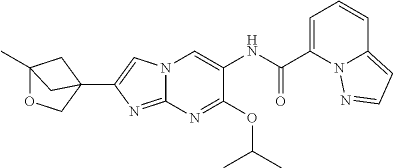 [SMILES: CC(C)Oc1nc2nc(C34COC(C)(C3)C4)cn2cc1NC(=O)c1cccc2ccnn12] |
| 346 | 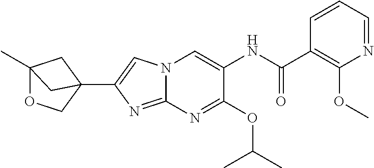 |
| 347 | 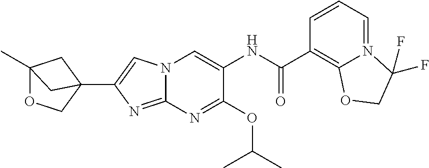 |
| 348 | 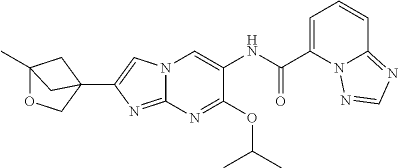 [SMILES: CC(C)Oc1nc2nc(C34COC(C)(C3)C4)cn2cc1NC(=O)c1cccc2ncnn12] |
| 349 | 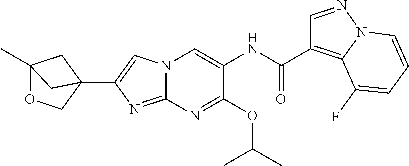 [SMILES: CC(C)Oc1nc2nc(C34COC(C)(C3)C4)cn2cc1NC(=O)c1cnn2cccc(F)c12] |
| 350 | 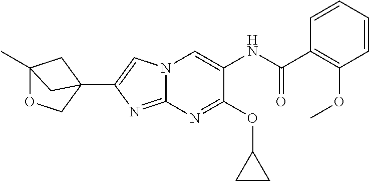 [SMILES: COc1ccccc1C(=O)Nc1cn2cc(C34COC(C)(C3)C4)nc2nc1OC1CC1] |
| 351 | 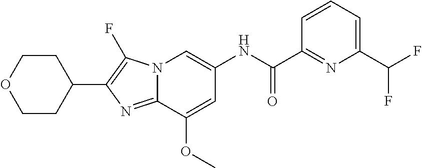 [SMILES: COc1cc(NC(=O)c2cccc(C(F)F)n2)cn2c(F)c(C3CCOCC3)nc12] |
| 352 | 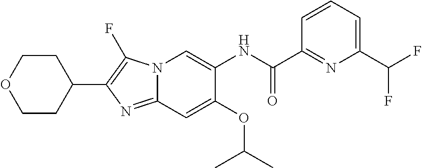 [SMILES: CC(C)Oc1cc2nc(C3CCOCC3)c(F)n2cc1NC(=O)c1cccc(C(F)F)n1] |
| 353 | 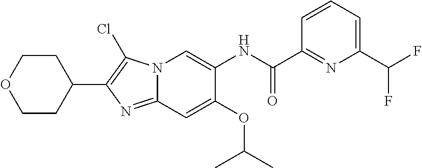 [SMILES: CC(C)Oc1cc2nc(C3CCOCC3)c(Cl)n2cc1NC(=O)c1cccc(C(F)F)n1] |
| 354 | 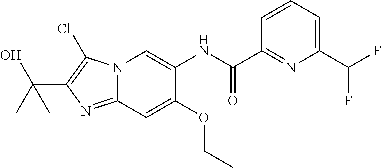 [SMILES: CCOc1cc2nc(C(C)(C)O)c(Cl)n2cc1NC(=O)c1cccc(C(F)F)n1] |
| 355 | 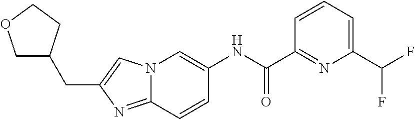 [SMILES: O=C(Nc1ccc2nc(CC3CCOC3)cn2c1)c1cccc(C(F)F)n1] |
| 356 | 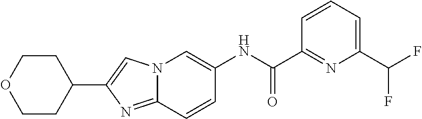 [SMILES: O=C(Nc1ccc2nc(C3CCOCC3)cn2c1)c1cccc(C(F)F)n1] |
| 357 | 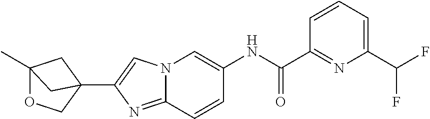 [SMILES: CC12CC(c3cn4cc(NC(=O)c5cccc(C(F)F)n5)ccc4n3)(CO1)C2] |
| 358 | 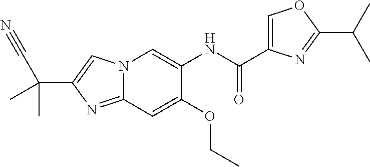 [SMILES: CCOc1cc2nc(C(C)(C)C#N)cn2cc1NC(=O)c1coc(C(C)C)n1] |
| 359 | 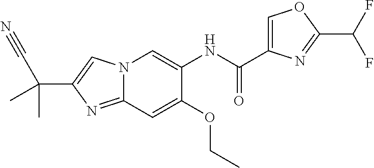 [SMILES: CCOc1cc2nc(C(C)(C)C#N)cn2cc1NC(=O)c1coc(C(F)F)n1] |
| 360 | 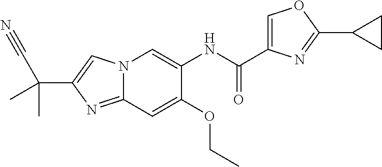 [SMILES: CCOc1cc2nc(C(C)(C)C#N)cn2cc1NC(=O)c1coc(C2CC2)n1] |
| 361 | 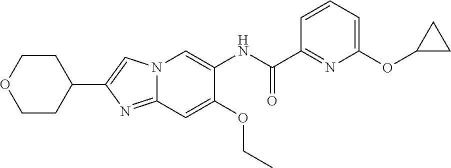 [SMILES: CCOc1cc2nc(C3CCOCC3)cn2cc1NC(=O)c1cccc(OC2CC2)n1] |
| 362 | 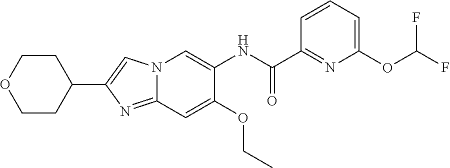 [SMILES: CCOc1cc2nc(C3CCOCC3)cn2cc1NC(=O)c1cccc(OC(F)F)n1] |
| 363 | 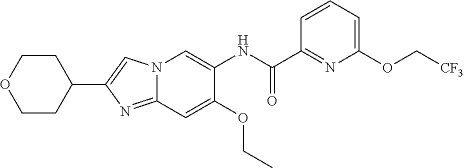 [SMILES: CCOc1cc2nc(C3CCOCC3)cn2cc1NC(=O)c1cccc(OCC(F)(F)F)n1] |
| 364 | 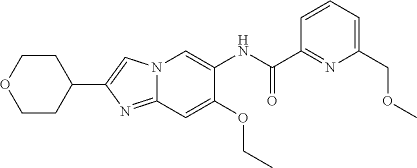 [SMILES: CCOc1cc2nc(C3CCOCC3)cn2cc1NC(=O)c1cccc(COC)n1] |
| 365 | 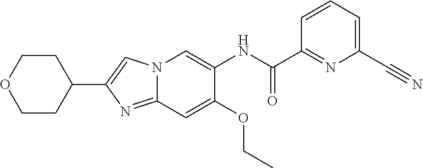 [SMILES: CCOc1cc2nc(C3CCOCC3)cn2cc1NC(=O)c1cccc(C#N)n1] |
| 366 | 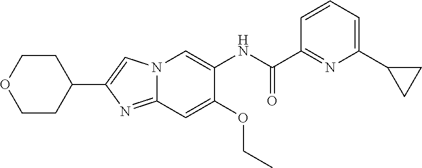 [SMILES: CCOc1cc2nc(C3CCOCC3)cn2cc1NC(=O)c1cccc(C2CC2)n1] |
| 367 | 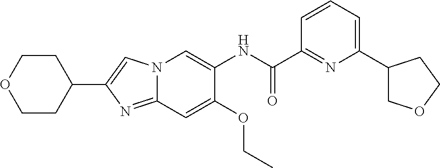 [SMILES: CCOc1cc2nc(C3CCOCC3)cn2cc1NC(=O)c1cccc(C2CCOC2)n1] |
| 368 | 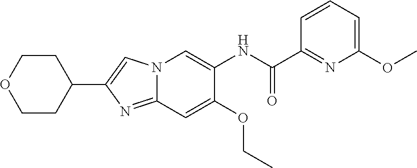 [SMILES: CCOc1cc2nc(C3CCOCC3)cn2cc1NC(=O)c1cccc(OC)n1] |
| 369 | 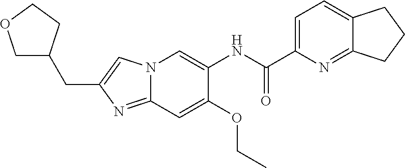 [SMILES: CCOc1cc2nc(CC3CCOC3)cn2cc1NC(=O)c1ccc2c(n1)CCC2] |
| 370 | 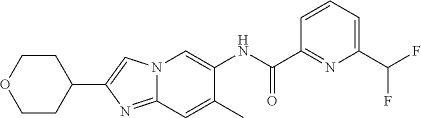 [SMILES: Cc1cc2nc(C3CCOCC3)cn2cc1NC(=O)c1cccc(C(F)F)n1] |
| 371 | 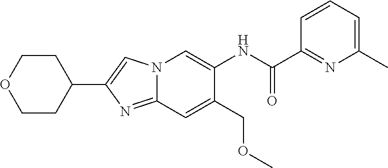 [SMILES: COCc1cc2nc(C3CCOCC3)cn2cc1NC(=O)c1cccc(C)n1] |
| 372 | 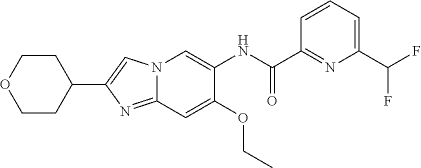 [SMILES: CCOc1cc2nc(C3CCOCC3)cn2cc1NC(=O)c1cccc(C(F)F)n1] |
| 373 | 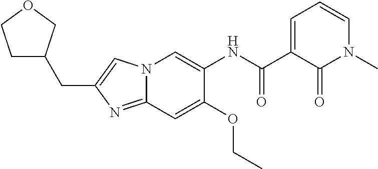 [SMILES: CCOc1cc2nc(CC3CCOC3)cn2cc1NC(=O)c1cccn(C)c1=O] |
| 374 | 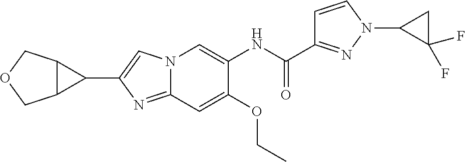 [SMILES: CCOc1cc2nc(C3C4COCC43)cn2cc1NC(=O)c1ccn(C2CC2(F)F)n1] |
| 375 | 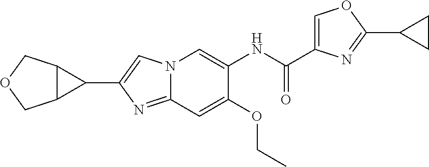 [SMILES: CCOc1cc2nc(C3C4COCC43)cn2cc1NC(=O)c1coc(C2CC2)n1] |
| 376 | 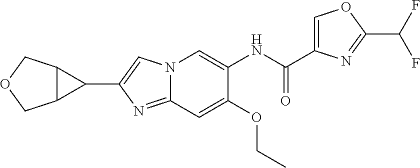 [SMILES: CCOc1cc2nc(C3C4COCC43)cn2cc1NC(=O)c1coc(C(F)F)n1] |
| 377 | 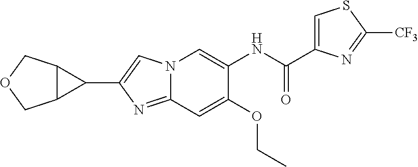 [SMILES: CCOc1cc2nc(C3C4COCC43)cn2cc1NC(=O)c1csc(C(F)(F)F)n1] |
| 378 | 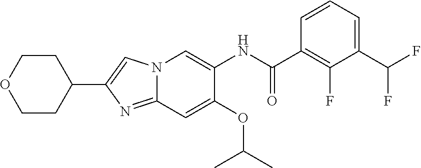 [SMILES: CC(C)Oc1cc2nc(C3CCOCC3)cn2cc1NC(=O)c1cccc(C(F)F)c1F] |
| 379 | 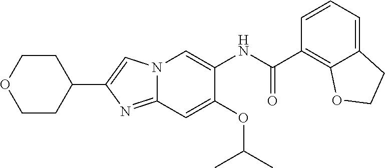 [SMILES: CC(C)Oc1cc2nc(C3CCOCC3)cn2cc1NC(=O)c1cccc2c1OCC2] |
| 380 | 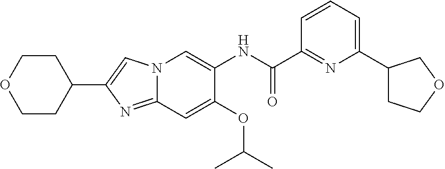 [SMILES: CC(C)Oc1cc2nc(C3CCOCC3)cn2cc1NC(=O)c1cccc(C2CCOC2)n1] |
| 381 | 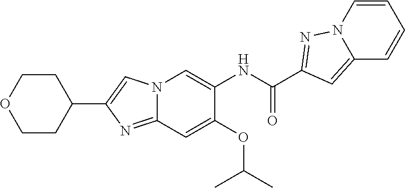 [SMILES: CC(C)Oc1cc2nc(C3CCOCC3)cn2cc1NC(=O)c1cc2ccccn2n1] |
| 382 | 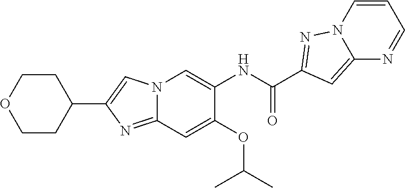 [SMILES: CC(C)Oc1cc2nc(C3CCOCC3)cn2cc1NC(=O)c1cc2ncccn2n1] |
| 383 | 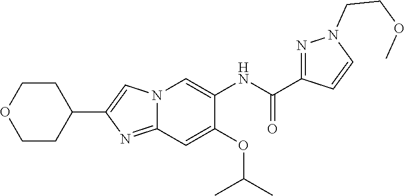 [SMILES: COCCn1ccc(C(=O)Nc2cn3cc(C4CCOCC4)nc3cc2OC(C)C)n1] |
| 384 | 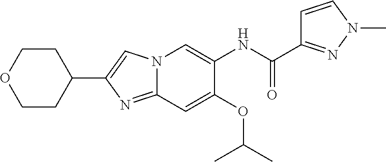 [SMILES: CC(C)Oc1cc2nc(C3CCOCC3)cn2cc1NC(=O)c1ccn(C)n1] |
| 385 | 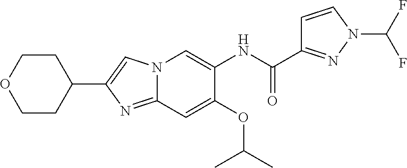 [SMILES: CC(C)Oc1cc2nc(C3CCOCC3)cn2cc1NC(=O)c1ccn(C(F)F)n1] |
| 386 | 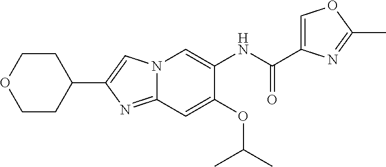 [SMILES: Cc1nc(C(=O)Nc2cn3cc(C4CCOCC4)nc3cc2OC(C)C)co1] |
| 387 | 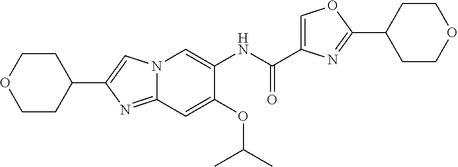 [SMILES: CC(C)Oc1cc2nc(C3CCOCC3)cn2cc1NC(=O)c1coc(C2CCOCC2)n1] |
| 388 | 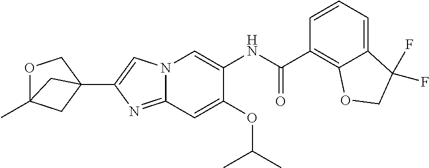 [SMILES: CC(C)Oc1cc2nc(C34COC(C)(C3)C4)cn2cc1NC(=O)c1cccc2c1OCC2(F)F] |
| 389 | 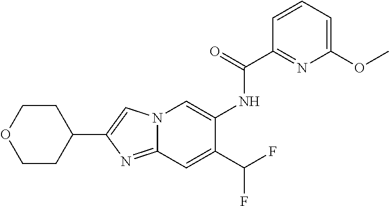 [SMILES: COc1cccc(C(=O)Nc2cn3cc(C4CCOCC4)nc3cc2C(F)F)n1] |
| 390 | 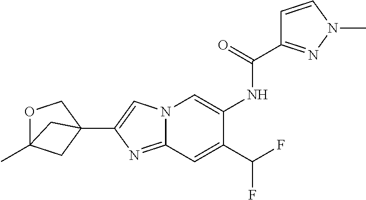 |
| 391 | 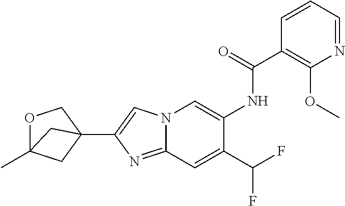 |
| 392 | 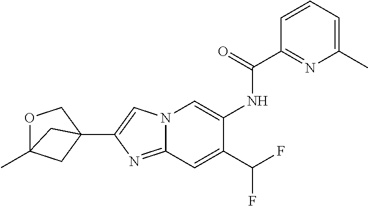 |
| 393 | 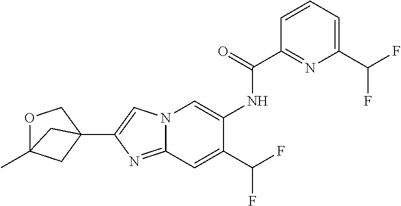 [SMILES: CC12CC(c3cn4cc(NC(=O)c5cccc(C(F)F)n5)c(C(F)F)cc4n3)(CO1)C2] |
| 394 | 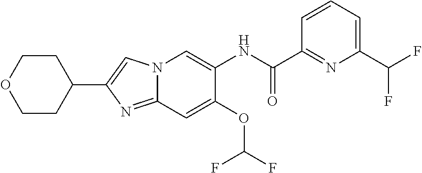 [SMILES: O=C(Nc1cn2cc(C3CCOCC3)nc2cc1OC(F)F)c1cccc(C(F)F)n1] |
| 395 | 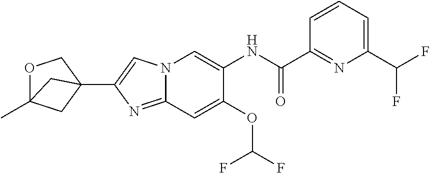 [SMILES: CC12CC(c3cn4cc(NC(=O)c5cccc(C(F)F)n5)c(OC(F)F)cc4n3)(CO1)C2] |
| 396 | 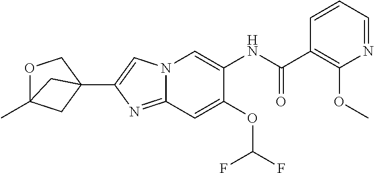 |
| 397 | 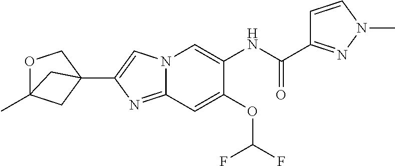 |
| 398 | 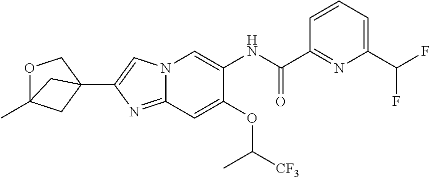 [SMILES: CC(Oc1cc2nc(C34COC(C)(C3)C4)cn2cc1NC(=O)c1cccc(C(F)F)n1)C(F)(F)F] |
| 399 | 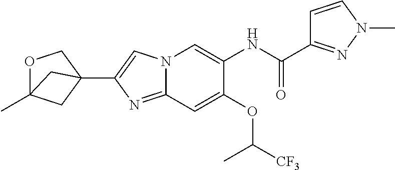 |
| 400 | 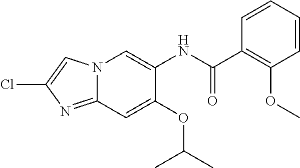 [SMILES: COc1ccccc1C(=O)Nc1cn2cc(Cl)nc2cc1OC(C)C] |
| 401 | 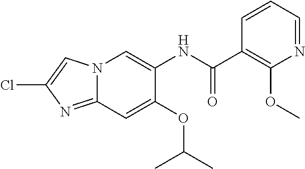 [SMILES: COc1ncccc1C(=O)Nc1cn2cc(Cl)nc2cc1OC(C)C] |
| 402 | 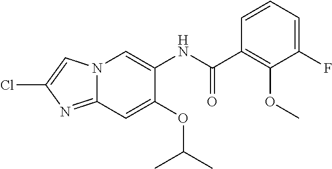 [SMILES: COc1c(F)cccc1C(=O)Nc1cn2cc(Cl)nc2cc1OC(C)C] |
| 403 | 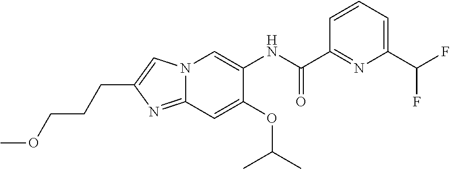 [SMILES: COCCCc1cn2cc(NC(=O)c3cccc(C(F)F)n3)c(OC(C)C)cc2n1] |
| 404 | 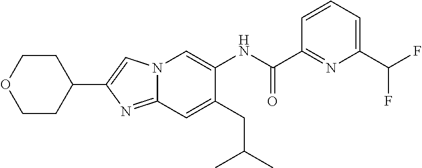 [SMILES: CC(C)Cc1cc2nc(C3CCOCC3)cn2cc1NC(=O)c1cccc(C(F)F)n1] |
| 405 | 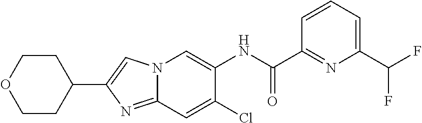 [SMILES: O=C(Nc1cn2cc(C3CCOCC3)nc2cc1Cl)c1cccc(C(F)F)n1] |
| 406 | 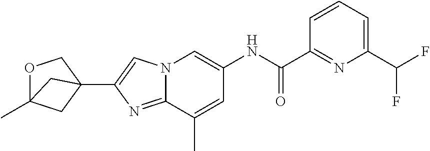 [SMILES: Cc1cc(NC(=O)c2cccc(C(F)F)n2)cn2cc(C34COC(C)(C3)C4)nc12] |
| 407 | 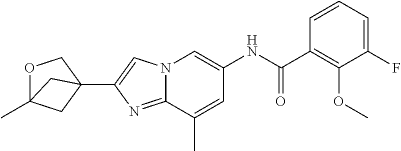 [SMILES: COc1c(F)cccc1C(=O)Nc1cc(C)c2nc(C34COC(C)(C3)C4)cn2c1] |
| 408 | 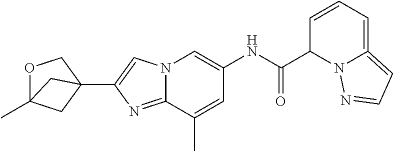 |
| 409 | 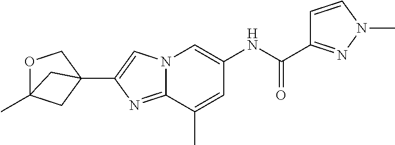 |
| 410 | 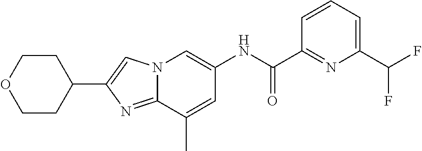 [SMILES: Cc1cc(NC(=O)c2cccc(C(F)F)n2)cn2cc(C3CCOCC3)nc12] |
| 411 | 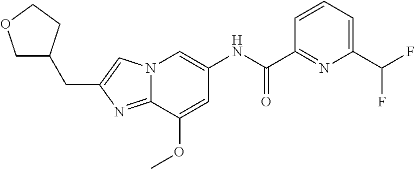 [SMILES: COc1cc(NC(=O)c2cccc(C(F)F)n2)cn2cc(CC3CCOC3)nc12] |
| 412 | 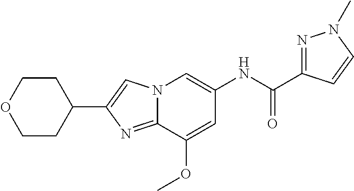 [SMILES: COc1cc(NC(=O)c2ccn(C)n2)cn2cc(C3CCOCC3)nc12] |
| 413 | 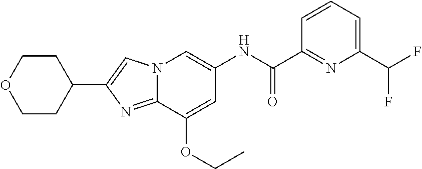 [SMILES: CCOc1cc(NC(=O)c2cccc(C(F)F)n2)cn2cc(C3CCOCC3)nc12] |
| 414 | 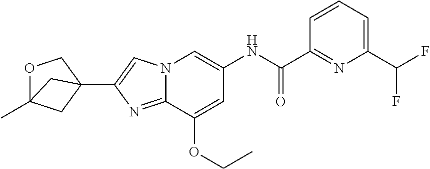 [SMILES: CCOc1cc(NC(=O)c2cccc(C(F)F)n2)cn2cc(C34COC(C)(C3)C4)nc12] |
| 415 | 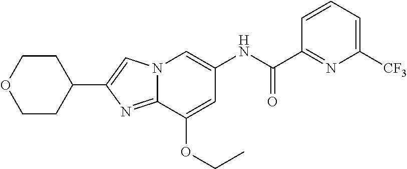 [SMILES: CCOc1cc(NC(=O)c2cccc(C(F)(F)F)n2)cn2cc(C3CCOCC3)nc12] |
| 416 | 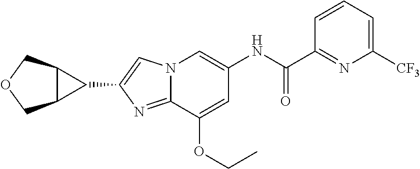 |
| 417 | 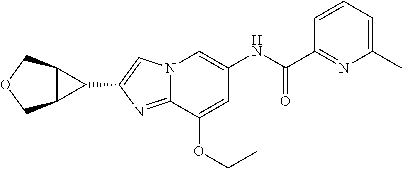 |
| 418 | 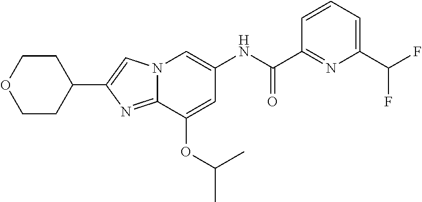 [SMILES: CC(C)Oc1cc(NC(=O)c2cccc(C(F)F)n2)cn2cc(C3CCOCC3)nc12] |
| 419 | 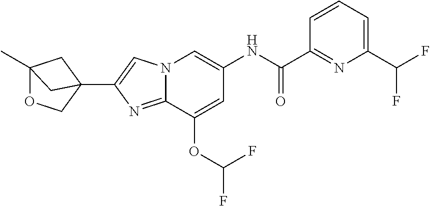 [SMILES: CC12CC(c3cn4cc(NC(=O)c5cccc(C(F)F)n5)cc(OC(F)F)c4n3)(CO1)C2] |
| 420 | 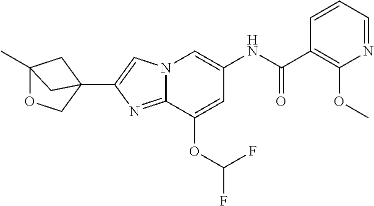 |
| 421 | 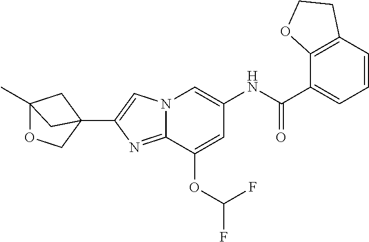 [SMILES: CC12CC(c3cn4cc(NC(=O)c5cccc6c5OCC6)cc(OC(F)F)c4n3)(CO1)C2] |
| 422 | 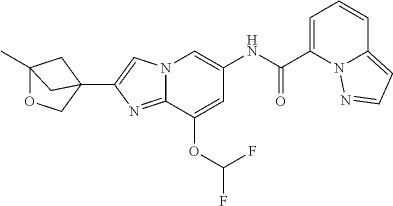 [SMILES: CC12CC(c3cn4cc(NC(=O)c5cccc6ccnn56)cc(OC(F)F)c4n3)(CO1)C2] |
| 423 | 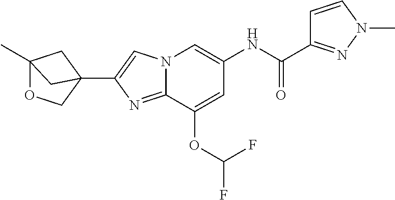 |
| 424 | 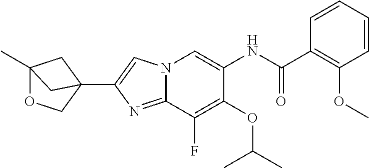 |
| 425 | 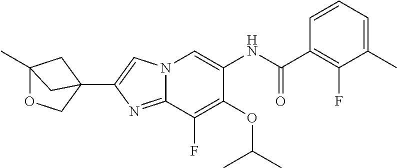 |
| 426 | 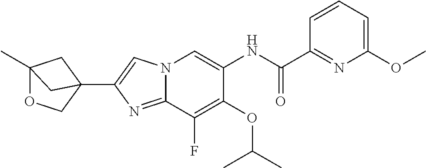 |
| 427 | 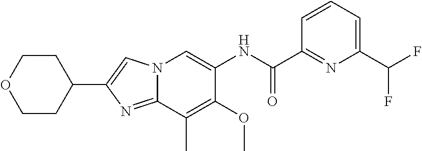 [SMILES: COc1c(NC(=O)c2cccc(C(F)F)n2)cn2cc(C3CCOCC3)nc2c1C] |
| 428 | 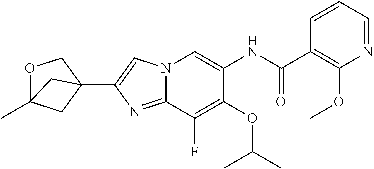 [SMILES: COc1ncccc1C(=O)Nc1cn2cc(C34COC(C)(C3)C4)nc2c(F)c1OC(C)C] |
| 429 | 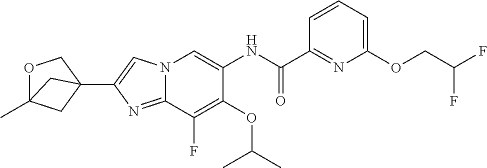 [SMILES: CC(C)Oc1c(NC(=O)c2cccc(OCC(F)F)n2)cn2cc(C34COC(C)(C3)C4)nc2c1F] |
| 430 | 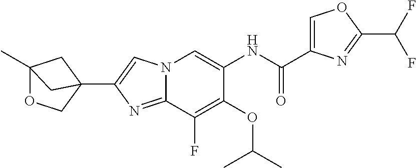 [SMILES: CC(C)Oc1c(NC(=O)c2coc(C(F)F)n2)cn2cc(C34COC(C)(C3)C4)nc2c1F] |
| 431 | 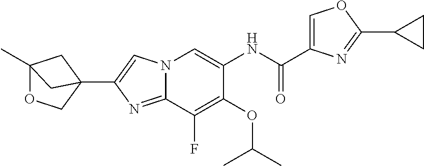 [SMILES: CC(C)Oc1c(NC(=O)c2coc(C3CC3)n2)cn2cc(C34COC(C)(C3)C4)nc2c1F] |
| 432 | 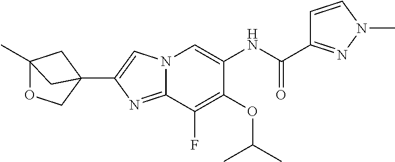 |
| 433 | 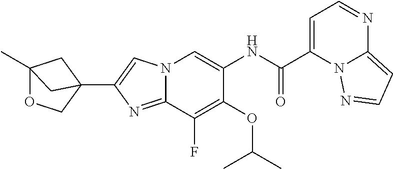 [SMILES: CC(C)Oc1c(NC(=O)c2ccnc3ccnn23)cn2cc(C34COC(C)(C3)C4)nc2c1F] |
| 434 | 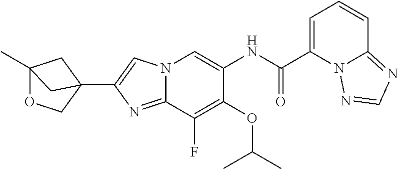 [SMILES: CC(C)Oc1c(NC(=O)c2cccc3ncnn23)cn2cc(C34COC(C)(C3)C4)nc2c1F] |
| 435 | 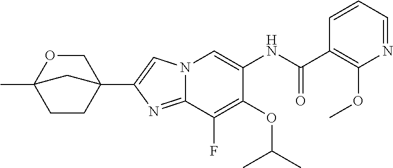 [SMILES: COc1ncccc1C(=O)Nc1cn2cc(C34CCC(C)(C3)OC4)nc2c(F)c1OC(C)C] |
| 436 | 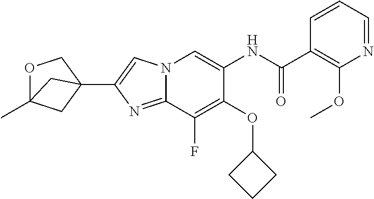 [SMILES: COc1ncccc1C(=O)Nc1cn2cc(C34COC(C)(C3)C4)nc2c(F)c1OC1CCC1] |
| 437 | 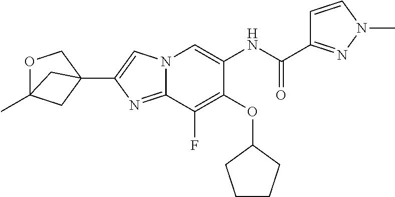 |
| 438 | 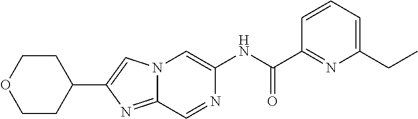 [SMILES: CCc1cccc(C(=O)Nc2cn3cc(C4CCOCC4)nc3cn2)n1] |
| 439 | 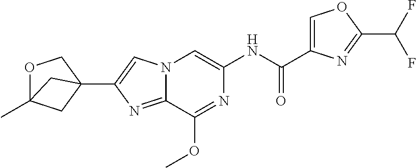 [SMILES: COc1nc(NC(=O)c2coc(C(F)F)n2)cn2cc(C34COC(C)(C3)C4)nc12] |
| 440 | 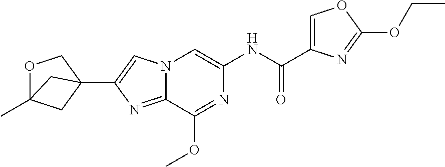 [SMILES: CCOc1nc(C(=O)Nc2cn3cc(C45COC(C)(C4)C5)nc3c(OC)n2)co1] |
| 441 | 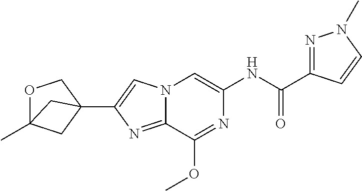 [SMILES: COc1nc(NC(=O)c2ccn(C)n2)cn2cc(C34COC(C)(C3)C4)nc12] |
| 442 | 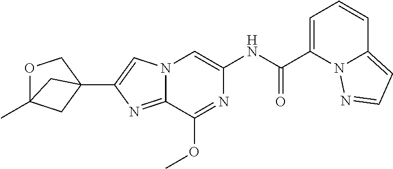 [SMILES: COc1nc(NC(=O)c2cccc3ccnn23)cn2cc(C34COC(C)(C3)C4)nc12] |
| 443 | 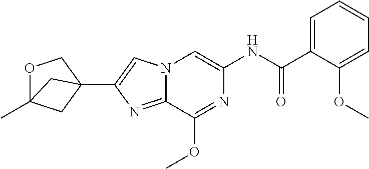 |
| 444 | 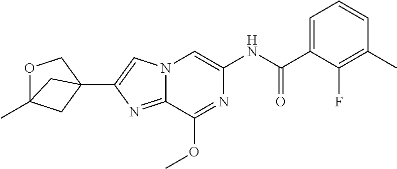 |
| 445 | 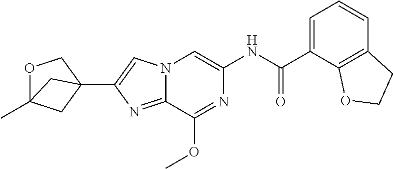 [SMILES: COc1nc(NC(=O)c2cccc3c2OCC3)cn2cc(C34COC(C)(C3)C4)nc12] |
| 446 | 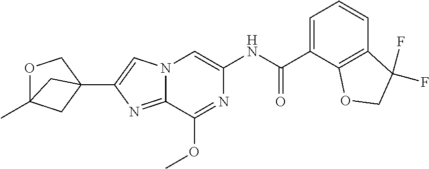 [SMILES: COc1nc(NC(=O)c2cccc3c2OCC3(F)F)cn2cc(C34COC(C)(C3)C4)nc12] |
| 447 | 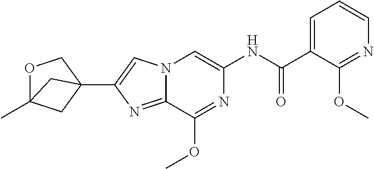 |
| 448 | 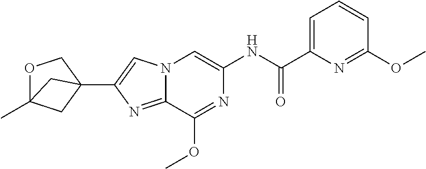 |
| 449 | 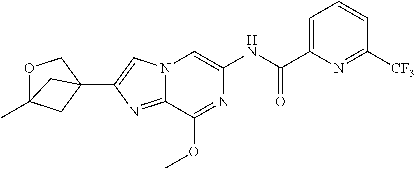 [SMILES: COc1nc(NC(=O)c2cccc(C(F)(F)F)n2)cn2cc(C34COC(C)(C3)C4)nc12] |
| 450 | 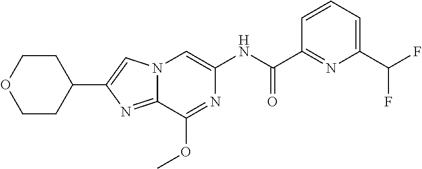 [SMILES: COc1nc(NC(=O)c2cccc(C(F)F)n2)cn2cc(C3CCOCC3)nc12] |
| 451 | 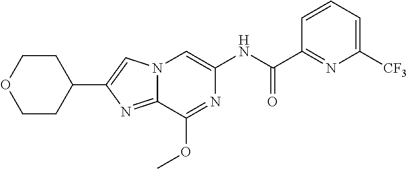 [SMILES: COc1nc(NC(=O)c2cccc(C(F)(F)F)n2)cn2cc(C3CCOCC3)nc12] |
| 452 | 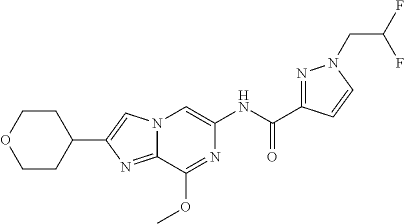 [SMILES: COc1nc(NC(=O)c2ccn(CC(F)F)n2)cn2cc(C3CCOCC3)nc12] |
| 453 | 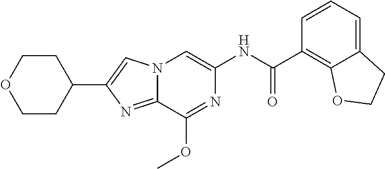 [SMILES: COc1nc(NC(=O)c2cccc3c2OCC3)cn2cc(C3CCOCC3)nc12] |
| 454 | 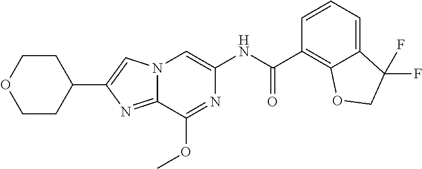 [SMILES: COc1nc(NC(=O)c2cccc3c2OCC3(F)F)cn2cc(C3CCOCC3)nc12] |
| 455 | 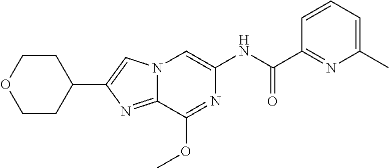 [SMILES: COc1nc(NC(=O)c2cccc(C)n2)cn2cc(C3CCOCC3)nc12] |
| 456 | 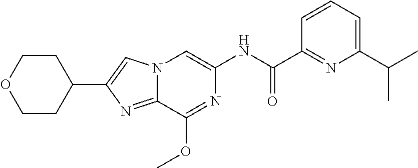 [SMILES: COc1nc(NC(=O)c2cccc(C(C)C)n2)cn2cc(C3CCOCC3)nc12] |
| 457 | 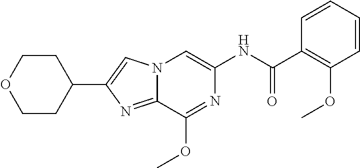 [SMILES: COc1ccccc1C(=O)Nc1cn2cc(C3CCOCC3)nc2c(OC)n1] |
| 458 | 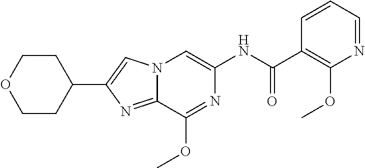 [SMILES: COc1ncccc1C(=O)Nc1cn2cc(C3CCOCC3)nc2c(OC)n1] |
| 459 | 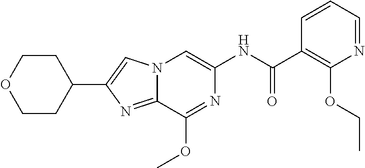 [SMILES: CCOc1ncccc1C(=O)Nc1cn2cc(C3CCOCC3)nc2c(OC)n1] |
| 460 | 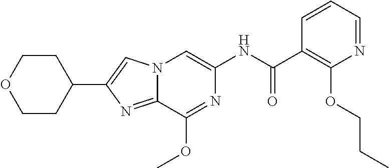 [SMILES: CCCOc1ncccc1C(=O)Nc1cn2cc(C3CCOCC3)nc2c(OC)n1] |
| 461 | 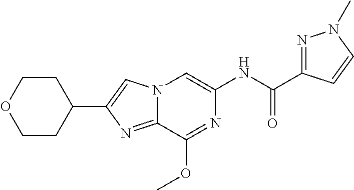 [SMILES: COc1nc(NC(=O)c2ccn(C)n2)cn2cc(C3CCOCC3)nc12] |
| 462 | 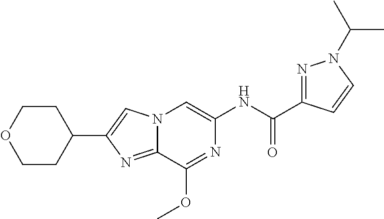 [SMILES: COc1nc(NC(=O)c2ccn(C(C)C)n2)cn2cc(C3CCOCC3)nc12] |
| 463 | 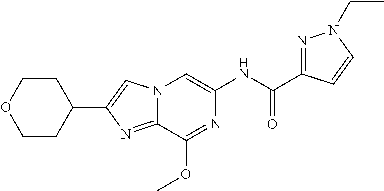 [SMILES: CCn1ccc(C(=O)Nc2cn3cc(C4CCOCC4)nc3c(OC)n2)n1] |
| 464 | 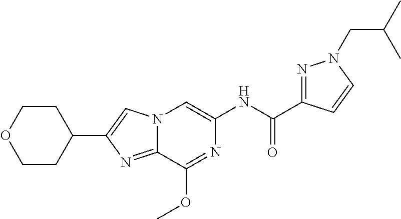 [SMILES: COc1nc(NC(=O)c2ccn(CC(C)C)n2)cn2cc(C3CCOCC3)nc12] |
| 465 | 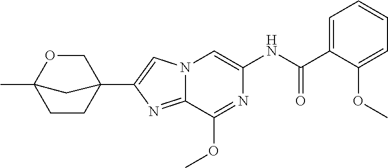 |
| 466 | 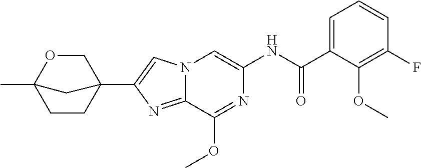 [SMILES: COc1c(F)cccc1C(=O)Nc1cn2cc(C34CCC(C)(C3)OC4)nc2c(OC)n1] |
| 467 | 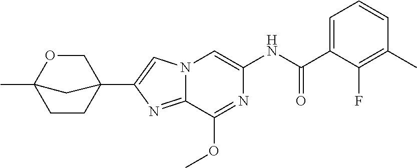 |
| 468 | 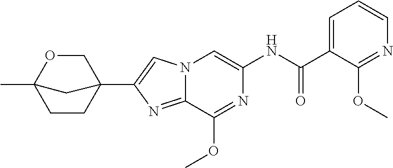 |
| 469 | 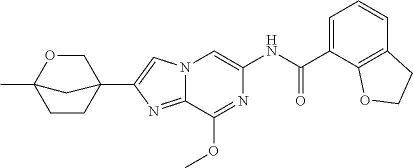 [SMILES: COc1nc(NC(=O)c2cccc3c2OCC3)cn2cc(C34CCC(C)(C3)OC4)nc12] |
| 470 | 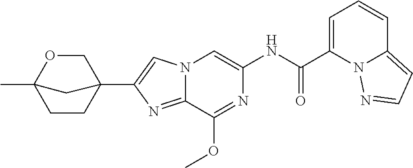 [SMILES: COc1nc(NC(=O)c2cccc3ccnn23)cn2cc(C34CCC(C)(C3)OC4)nc12] |
| 471 | 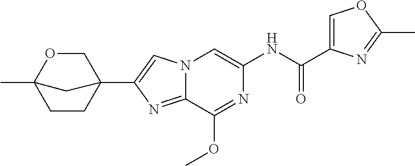 |
| 472 | 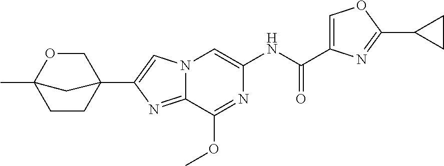 [SMILES: COc1nc(NC(=O)c2coc(C3CC3)n2)cn2cc(C34CCC(C)(C3)OC4)nc12] |
| 473 | 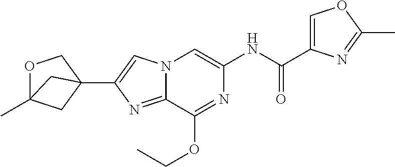 |
| 474 | 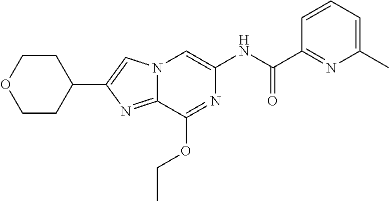 [SMILES: CCOc1nc(NC(=O)c2cccc(C)n2)cn2cc(C3CCOCC3)nc12] |
| 475 | 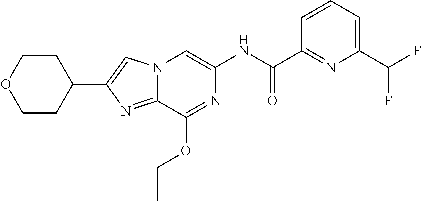 [SMILES: CCOc1nc(NC(=O)c2cccc(C(F)F)n2)cn2cc(C3CCOCC3)nc12] |
| 476 | 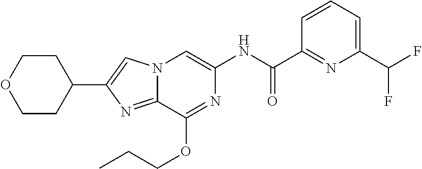 [SMILES: CCCOc1nc(NC(=O)c2cccc(C(F)F)n2)cn2cc(C3CCOCC3)nc12] |
| 477 | 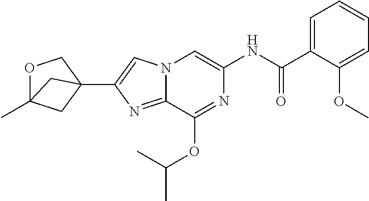 |
| 478 | 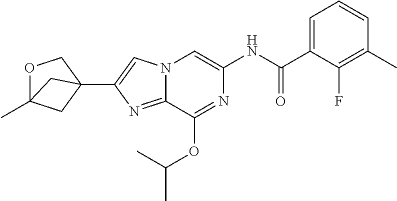 |
| 479 | 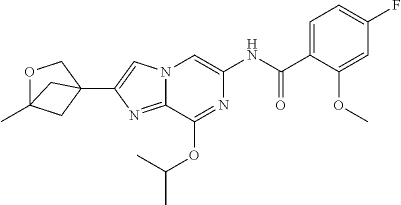 [SMILES: COc1cc(F)ccc1C(=O)Nc1cn2cc(C34COC(C)(C3)C4)nc2c(OC(C)C)n1] |
| 480 | 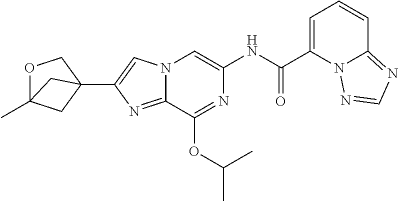 [SMILES: CC(C)Oc1nc(NC(=O)c2cccc3ncnn23)cn2cc(C34COC(C)(C3)C4)nc12] |
| 481 | 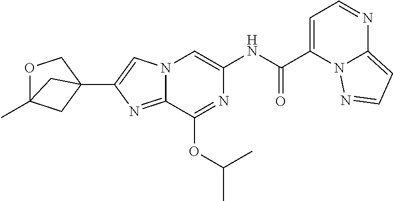 [SMILES: CC(C)Oc1nc(NC(=O)c2ccnc3ccnn23)cn2cc(C34COC(C)(C3)C4)nc12] |
| 482 | 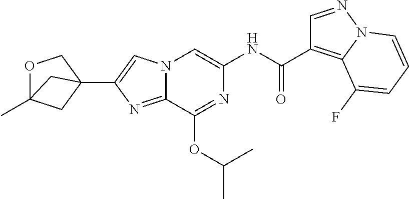 [SMILES: CC(C)Oc1nc(NC(=O)c2cnn3cccc(F)c23)cn2cc(C34COC(C)(C3)C4)nc12] |
| 483 | 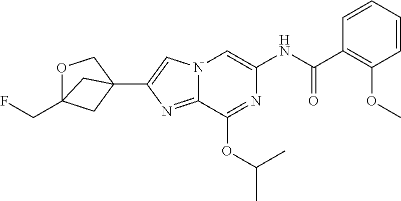 [SMILES: COc1ccccc1C(=O)Nc1cn2cc(C34COC(CF)(C3)C4)nc2c(OC(C)C)n1] |
| 484 | 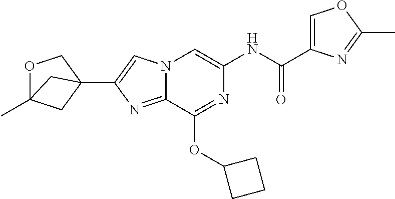 |
| 485 | 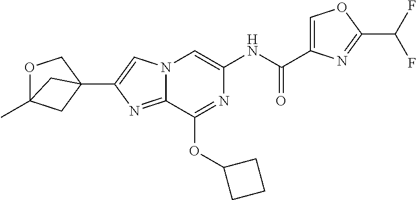 [SMILES: CC12CC(c3cn4cc(NC(=O)c5coc(C(F)F)n5)nc(OC5CCC5)c4n3)(CO1)C2] |
| 486 | 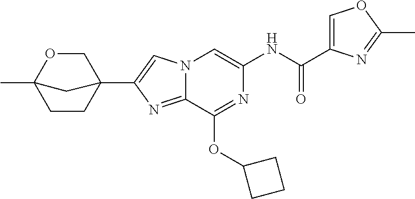 |
| 487 | 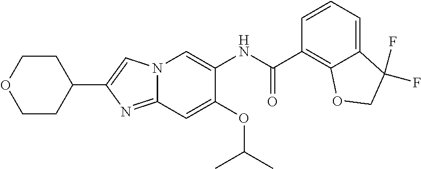 [SMILES: CC(C)Oc1cc2nc(C3CCOCC3)cn2cc1NC(=O)c1cccc2c1OCC2(F)F] |
| 488 | 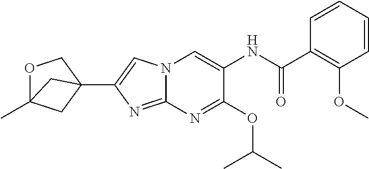 |
| 489 | 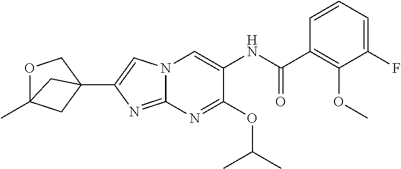 [SMILES: COc1c(F)cccc1C(=O)Nc1cn2cc(C34COC(C)(C3)C4)nc2nc1OC(C)C] |
| 490 | 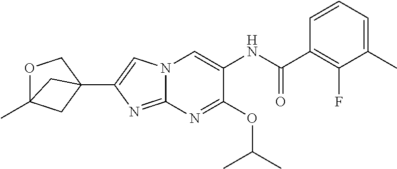 |
| 491 | 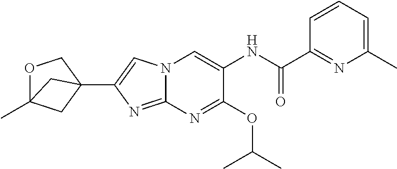 |
| 492 | 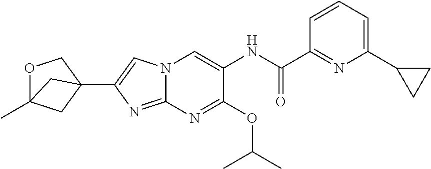 [SMILES: CC(C)Oc1nc2nc(C34COC(C)(C3)C4)cn2cc1NC(=O)c1cccc(C2CC2)n1] |
| 493 | 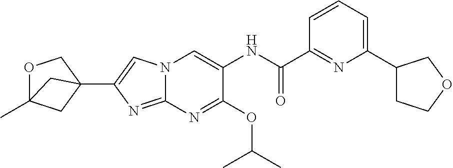 [SMILES: CC(C)Oc1nc2nc(C34COC(C)(C3)C4)cn2cc1NC(=O)c1cccc(C2CCOC2)n1] |
| 494 | 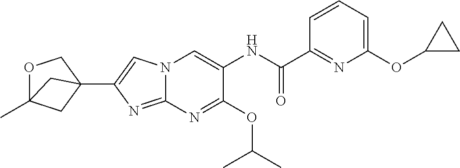 [SMILES: CC(C)Oc1nc2nc(C34COC(C)(C3)C4)cn2cc1NC(=O)c1cccc(OC2CC2)n1] |
| 495 | 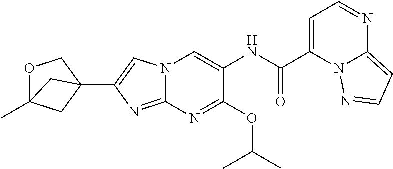 [SMILES: CC(C)Oc1nc2nc(C34COC(C)(C3)C4)cn2cc1NC(=O)c1ccnc2ccnn12] |
| 496 | 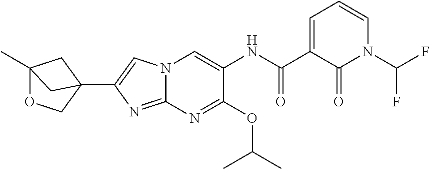 [SMILES: CC(C)Oc1nc2nc(C34COC(C)(C3)C4)cn2cc1NC(=O)c1cccn(C(F)F)c1=O] |
| 497 | 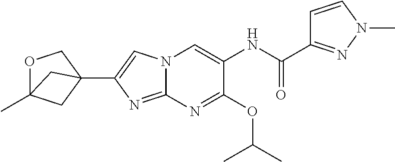 |
| 498 | 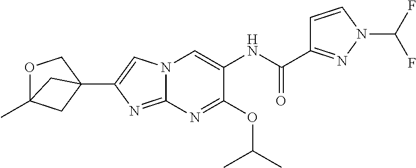 [SMILES: CC(C)Oc1nc2nc(C34COC(C)(C3)C4)cn2cc1NC(=O)c1ccn(C(F)F)n1] |
| 499 | 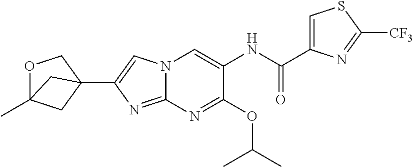 [SMILES: CC(C)Oc1nc2nc(C34COC(C)(C3)C4)cn2cc1NC(=O)c1csc(C(F)(F)F)n1] |
| 500 | 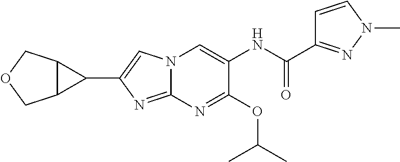 [SMILES: CC(C)Oc1nc2nc(C3C4COCC43)cn2cc1NC(=O)c1ccn(C)n1] |
| 501 | 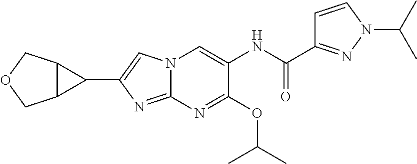 [SMILES: CC(C)Oc1nc2nc(C3C4COCC43)cn2cc1NC(=O)c1ccn(C(C)C)n1] |
| 502 | 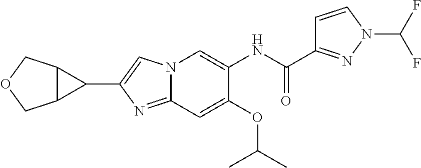 [SMILES: CC(C)Oc1cc2nc(C3C4COCC43)cn2cc1NC(=O)c1ccn(C(F)F)n1] |
| 503 | 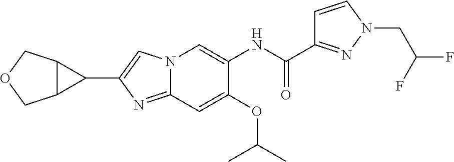 [SMILES: CC(C)Oc1cc2nc(C3C4COCC43)cn2cc1NC(=O)c1ccn(CC(F)F)n1] |
| 504 | 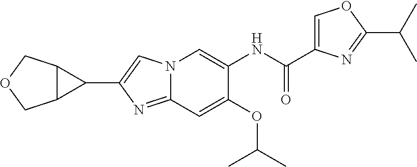 [SMILES: CC(C)Oc1cc2nc(C3C4COCC43)cn2cc1NC(=O)c1coc(C(C)C)n1] |
| 505 | 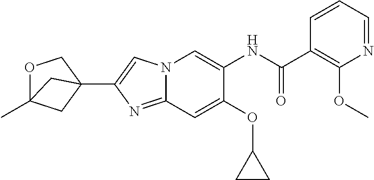 |
| 506 | 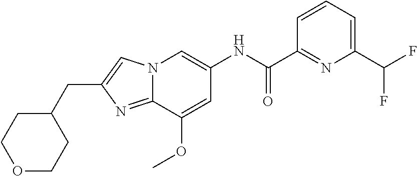 [SMILES: COc1cc(NC(=O)c2cccc(C(F)F)n2)cn2cc(CC3CCOCC3)nc12] |
| 507 | 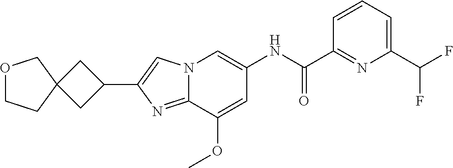 [SMILES: COc1cc(NC(=O)c2cccc(C(F)F)n2)cn2cc(C3CC4(CCOC4)C3)nc12] |
| 508 | 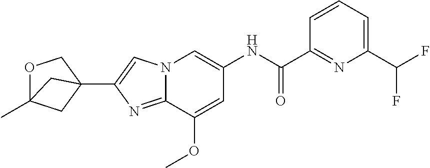 [SMILES: COc1cc(NC(=O)c2cccc(C(F)F)n2)cn2cc(C34COC(C)(C3)C4)nc12] |
| 509 | 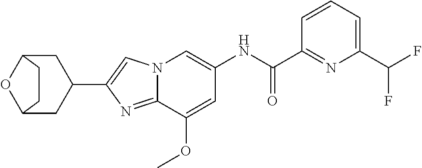 [SMILES: COc1cc(NC(=O)c2cccc(C(F)F)n2)cn2cc(C3CC4CCC(C3)O4)nc12] |
| 510 | 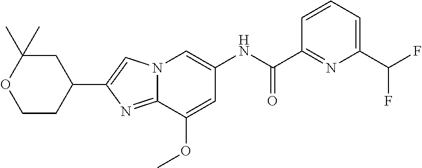 [SMILES: COc1cc(NC(=O)c2cccc(C(F)F)n2)cn2cc(C3CCOC(C)(C)C3)nc12] |
| 511 | 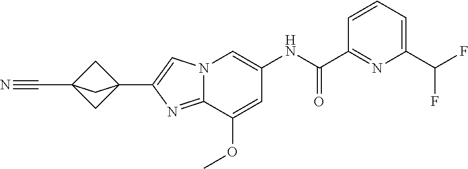 [SMILES: COc1cc(NC(=O)c2cccc(C(F)F)n2)cn2cc(C34CC(C#N)(C3)C4)nc12] |
| 512 | 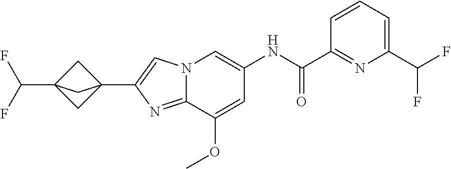 [SMILES: COc1cc(NC(=O)c2cccc(C(F)F)n2)cn2cc(C34CC(C(F)F)(C3)C4)nc12] |
| 513 | 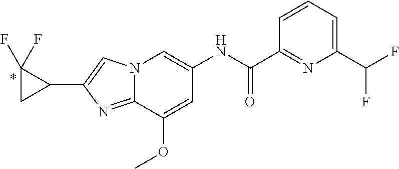 [SMILES: COc1cc(NC(=O)c2cccc(C(F)F)n2)cn2cc(C3CC3(F)F)nc12] |
| 514 | 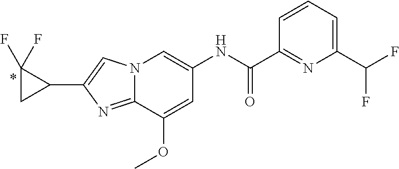 [SMILES: COc1cc(NC(=O)c2cccc(C(F)F)n2)cn2cc(C3CC3(F)F)nc12] |
| 515 | 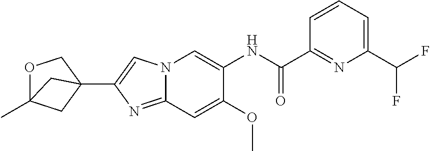 [SMILES: COc1cc2nc(C34COC(C)(C3)C4)cn2cc1NC(=O)c1cccc(C(F)F)n1] |
| 516 | 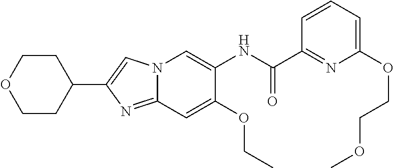 [SMILES: CCOc1cc2nc(C3CCOCC3)cn2cc1NC(=O)c1cccc(OCCOC)n1] |
| 517 | 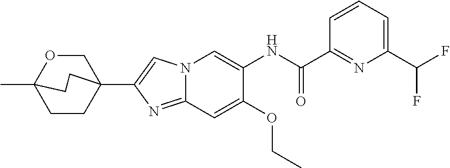 [SMILES: CCOc1cc2nc(C34CCC(C)(CC3)OC4)cn2cc1NC(=O)c1cccc(C(F)F)n1] |
| 518 | 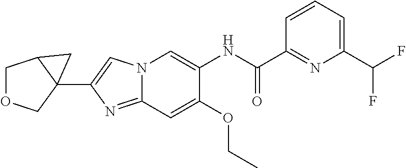 [SMILES: CCOc1cc2nc(C34COCC3C4)cn2cc1NC(=O)c1cccc(C(F)F)n1] |
| 519 | 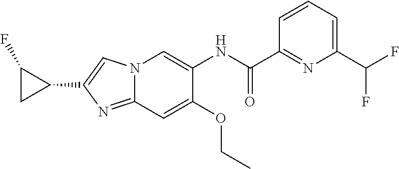 [SMILES: CCOc1cc2nc([C@@H]3C[C@@H]3F)cn2cc1NC(=O)c1cccc(C(F)F)n1] |
| 520 | 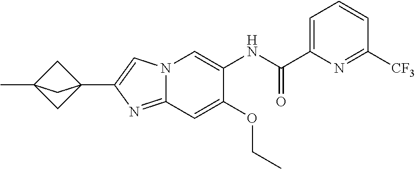 [SMILES: CCOc1cc2nc(C34CC(C)(C3)C4)cn2cc1NC(=O)c1cccc(C(F)(F)F)n1] |
| 521 | 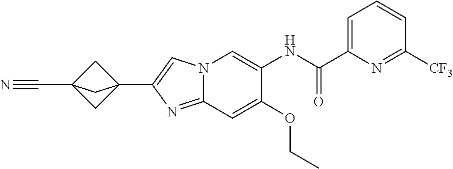 [SMILES: CCOc1cc2nc(C34CC(C#N)(C3)C4)cn2cc1NC(=O)c1cccc(C(F)(F)F)n1] |
| 522 | 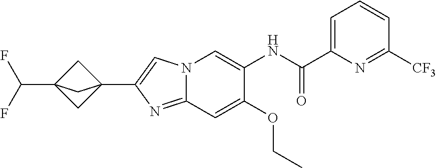 [SMILES: CCOc1cc2nc(C34CC(C(F)F)(C3)C4)cn2cc1NC(=O)c1cccc(C(F)(F)F)n1] |
| 523 | 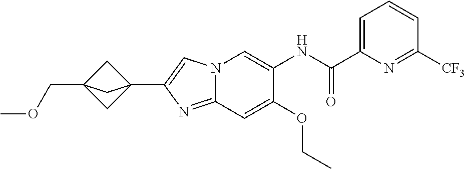 [SMILES: CCOc1cc2nc(C34CC(COC)(C3)C4)cn2cc1NC(=O)c1cccc(C(F)(F)F)n1] |
| 524 | 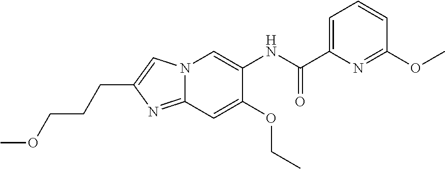 |
| 525 | 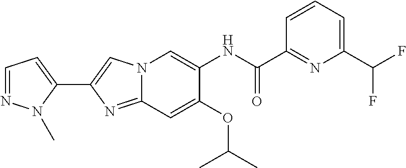 [SMILES: CC(C)Oc1cc2nc(-c3ccnn3C)cn2cc1NC(=O)c1cccc(C(F)F)n1] |
| 526 | 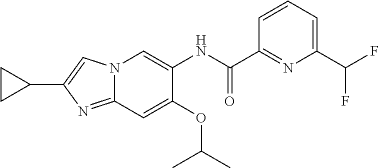 [SMILES: CC(C)Oc1cc2nc(C3CC3)cn2cc1NC(=O)c1cccc(C(F)F)n1] |
| 527 | 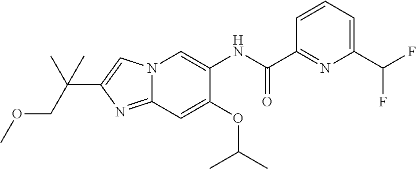 [SMILES: COCC(C)(C)c1cn2cc(NC(=O)c3cccc(C(F)F)n3)c(OC(C)C)cc2n1] |
| 528 | 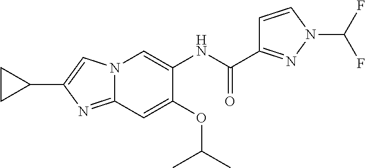 [SMILES: CC(C)Oc1cc2nc(C3CC3)cn2cc1NC(=O)c1ccn(C(F)F)n1] |
| 529 | 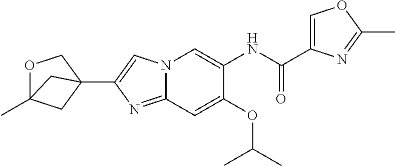 |
| 530 | 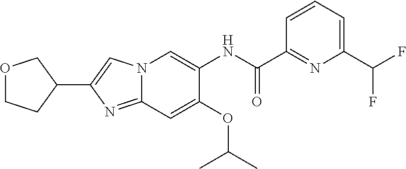 [SMILES: CC(C)Oc1cc2nc(C3CCOC3)cn2cc1NC(=O)c1cccc(C(F)F)n1] |
| 531 | 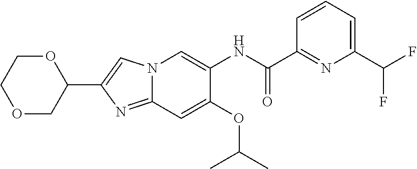 [SMILES: CC(C)Oc1cc2nc(C3COCCO3)cn2cc1NC(=O)c1cccc(C(F)F)n1] |
| 532 | 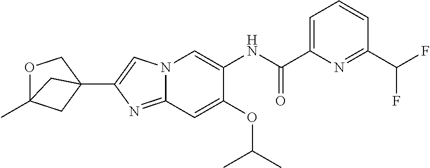 [SMILES: CC(C)Oc1cc2nc(C34COC(C)(C3)C4)cn2cc1NC(=O)c1cccc(C(F)F)n1] |
| 533 | 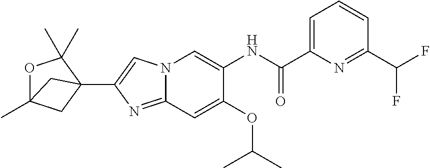 [SMILES: CC(C)Oc1cc2nc(C34CC(C)(C3)OC4(C)C)cn2cc1NC(=O)c1cccc(C(F)F)n1] |
| 534 | 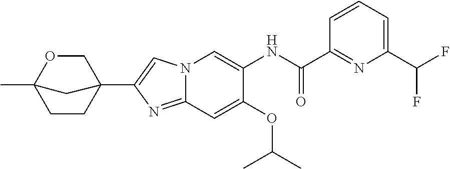 [SMILES: CC(C)Oc1cc2nc(C34CCC(C)(C3)OC4)cn2cc1NC(=O)c1cccc(C(F)F)n1] |
| 535 | 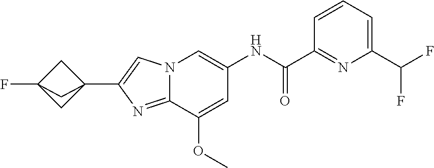 [SMILES: COc1cc(NC(=O)c2cccc(C(F)F)n2)cn2cc(C34CC(F)(C3)C4)nc12] |
| 536 | 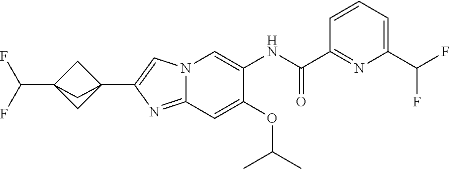 [SMILES: CC(C)Oc1cc2nc(C34CC(C(F)F)(C3)C4)cn2cc1NC(=O)c1cccc(C(F)F)n1] |
| 537 | 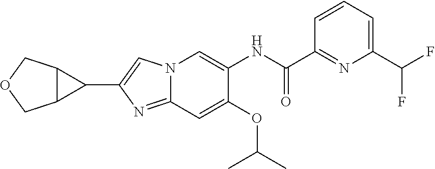 [SMILES: CC(C)Oc1cc2nc(C3C4COCC43)cn2cc1NC(=O)c1cccc(C(F)F)n1] |
| 538 | 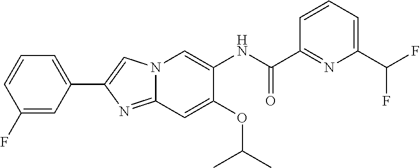 [SMILES: CC(C)Oc1cc2nc(-c3cccc(F)c3)cn2cc1NC(=O)c1cccc(C(F)F)n1] |
| 539 | 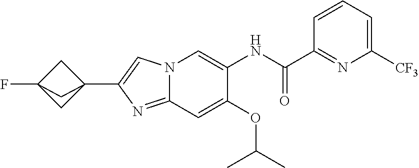 [SMILES: CC(C)Oc1cc2nc(C34CC(F)(C3)C4)cn2cc1NC(=O)c1cccc(C(F)(F)F)n1] |
| 540 | 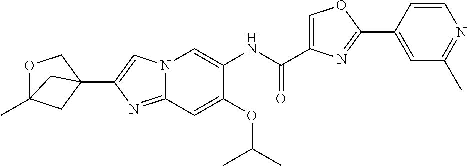 |
| 541 | 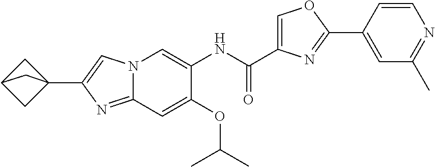 [SMILES: Cc1cc(-c2nc(C(=O)Nc3cn4cc(C56CC(C5)C6)nc4cc3OC(C)C)co2)ccn1] |
| 542 | 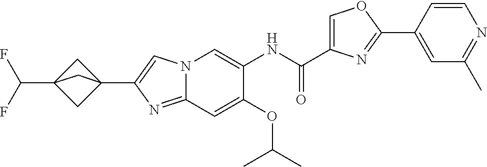 [SMILES: Cc1cc(-c2nc(C(=O)Nc3cn4cc(C56CC(C(F)F)(C5)C6)nc4cc3OC(C)C)co2)ccn1] |
| 543 | 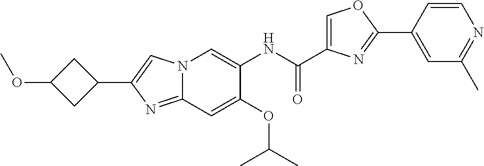 |
| 544 | 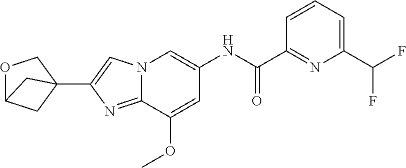 [SMILES: COc1cc(NC(=O)c2cccc(C(F)F)n2)cn2cc(C34COC(C3)C4)nc12] |
| 545 | 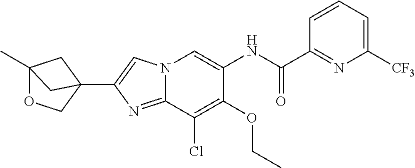 [SMILES: CCOc1c(NC(=O)c2cccc(C(F)(F)F)n2)cn2cc(C34COC(C)(C3)C4)nc2c1Cl] |
| 546 | 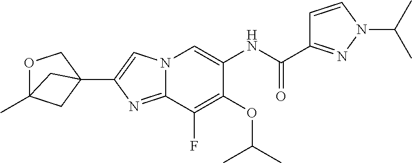 [SMILES: CC(C)Oc1c(NC(=O)c2ccn(C(C)C)n2)cn2cc(C34COC(C)(C3)C4)nc2c1F] |
| 547 | 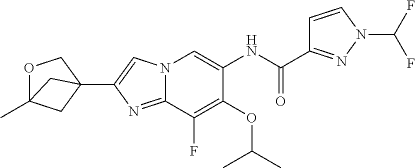 [SMILES: CC(C)Oc1c(NC(=O)c2ccn(C(F)F)n2)cn2cc(C34COC(C)(C3)C4)nc2c1F] |
| 548 | 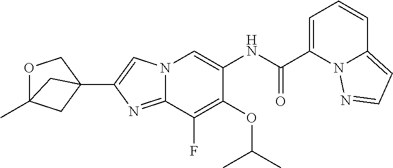 [SMILES: CC(C)Oc1c(NC(=O)c2cccc3ccnn23)cn2cc(C34COC(C)(C3)C4)nc2c1F] |
| 549 | 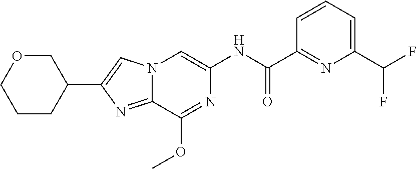 [SMILES: COc1nc(NC(=O)c2cccc(C(F)F)n2)cn2cc(C3CCCOC3)nc12] |
| 550 | 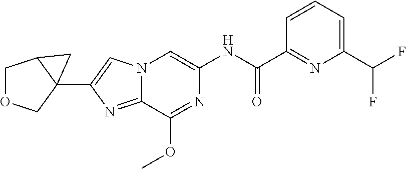 [SMILES: COc1nc(NC(=O)c2cccc(C(F)F)n2)cn2cc(C34COCC3C4)nc12] |
| 551 | 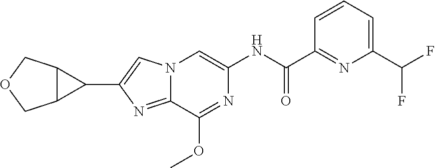 [SMILES: COc1nc(NC(=O)c2cccc(C(F)F)n2)cn2cc(C3C4COCC43)nc12] |
| 552 | 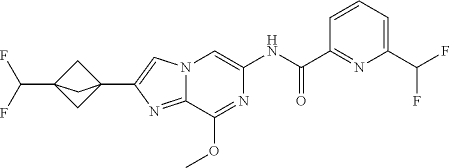 [SMILES: COc1nc(NC(=O)c2cccc(C(F)F)n2)cn2cc(C34CC(C(F)F)(C3)C4)nc12] |
| 553 | 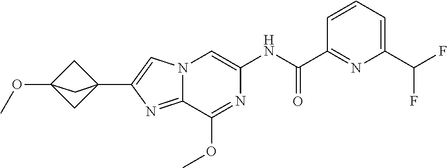 [SMILES: COc1nc(NC(=O)c2cccc(C(F)F)n2)cn2cc(C34CC(OC)(C3)C4)nc12] |
| 554 | 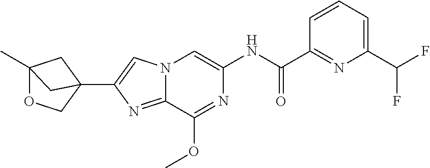 [SMILES: COc1nc(NC(=O)c2cccc(C(F)F)n2)cn2cc(C34COC(C)(C3)C4)nc12] |
| 555 | 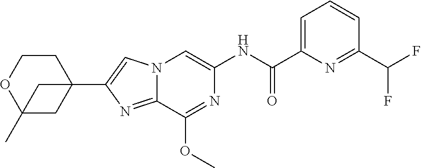 [SMILES: COc1nc(NC(=O)c2cccc(C(F)F)n2)cn2cc(C34CCOC(C)(C3)C4)nc12] |
| 556 | 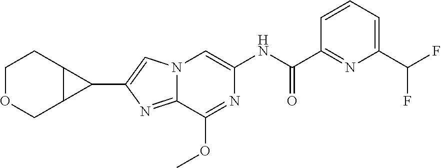 [SMILES: COc1nc(NC(=O)c2cccc(C(F)F)n2)cn2cc(C3C4CCOCC43)nc12] |
| 557 | 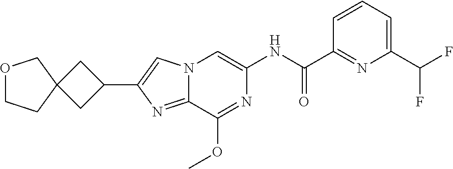 [SMILES: COc1nc(NC(=O)c2cccc(C(F)F)n2)cn2cc(C3CC4(CCOC4)C3)nc12] |
| 558 | 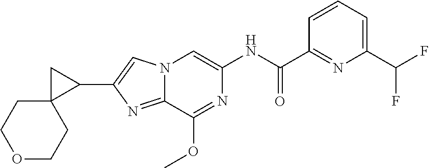 [SMILES: COc1nc(NC(=O)c2cccc(C(F)F)n2)cn2cc(C3CC34CCOCC4)nc12] |
| 559 | 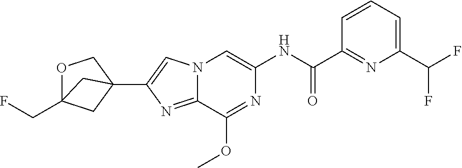 [SMILES: COc1nc(NC(=O)c2cccc(C(F)F)n2)cn2cc(C34COC(CF)(C3)C4)nc12] |
| 560 | 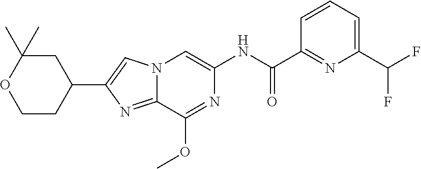 [SMILES: COc1nc(NC(=O)c2cccc(C(F)F)n2)cn2cc(C3CCOC(C)(C)C3)nc12] |
| 561 | 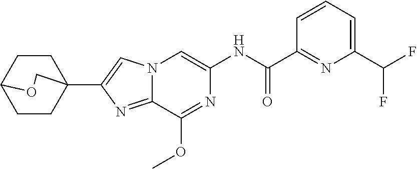 [SMILES: COc1nc(NC(=O)c2cccc(C(F)F)n2)cn2cc(C34CCC(CC3)OC4)nc12] |
| 562 | 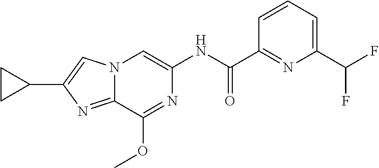 [SMILES: COc1nc(NC(=O)c2cccc(C(F)F)n2)cn2cc(C3CC3)nc12] |
| 563 | 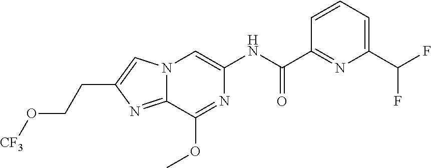 [SMILES: COc1nc(NC(=O)c2cccc(C(F)F)n2)cn2cc(CCOC(F)(F)F)nc12] |
| 564 | 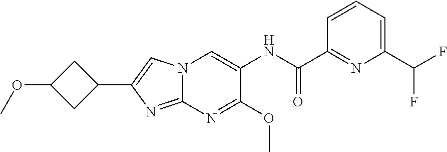 [SMILES: COc1nc2nc(C3CC(OC)C3)cn2cc1NC(=O)c1cccc(C(F)F)n1] |
| 565 | 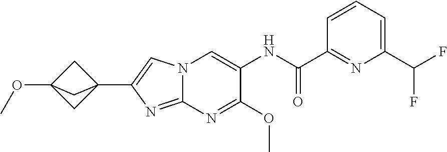 [SMILES: COc1nc2nc(C34CC(OC)(C3)C4)cn2cc1NC(=O)c1cccc(C(F)F)n1] |
| 566 | 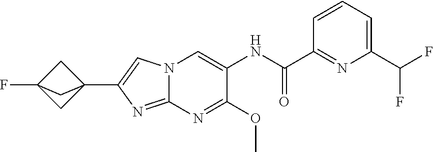 [SMILES: COc1nc2nc(C34CC(F)(C3)C4)cn2cc1NC(=O)c1cccc(C(F)F)n1] |
| 567 | 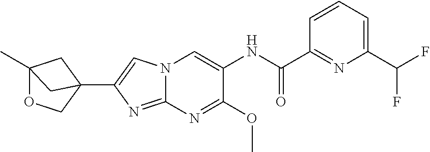 [SMILES: COc1nc2nc(C34COC(C)(C3)C4)cn2cc1NC(=O)c1cccc(C(F)F)n1] |
| 568 | 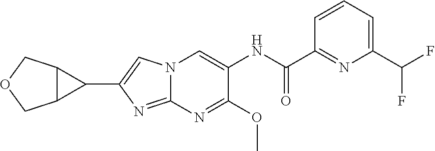 [SMILES: COc1nc2nc(C3C4COCC43)cn2cc1NC(=O)c1cccc(C(F)F)n1] |
| 569 | 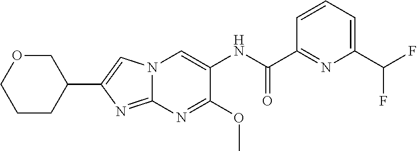 [SMILES: COc1nc2nc(C3CCCOC3)cn2cc1NC(=O)c1cccc(C(F)F)n1] |
| 570 | 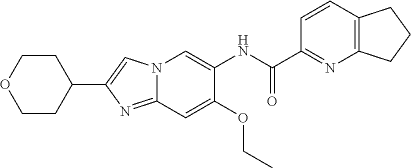 [SMILES: CCOc1cc2nc(C3CCOCC3)cn2cc1NC(=O)c1ccc2c(n1)CCC2] |
| 571 | 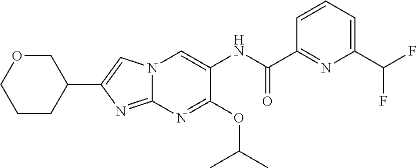 [SMILES: CC(C)Oc1nc2nc(C3CCCOC3)cn2cc1NC(=O)c1cccc(C(F)F)n1] |
| 572 | 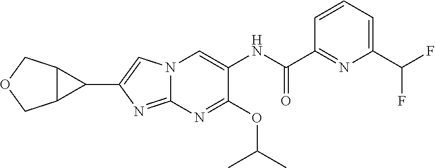 [SMILES: CC(C)Oc1nc2nc(C3C4COCC43)cn2cc1NC(=O)c1cccc(C(F)F)n1] |
| 573 | 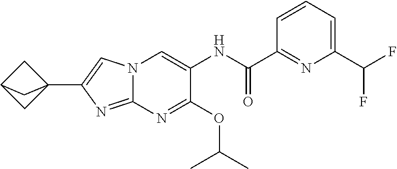 [SMILES: CC(C)Oc1nc2nc(C34CC(C3)C4)cn2cc1NC(=O)c1cccc(C(F)F)n1] |
| 574 | 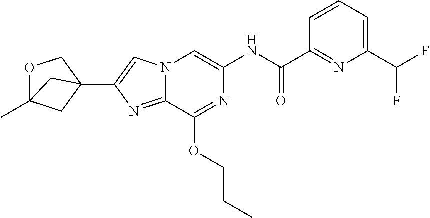 [SMILES: CCCOc1nc(NC(=O)c2cccc(C(F)F)n2)cn2cc(C34COC(C)(C3)C4)nc12] |
| 575 | 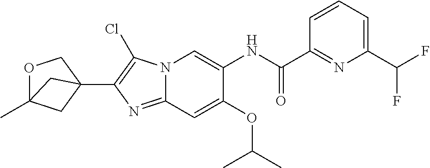 [SMILES: CC(C)Oc1cc2nc(C34COC(C)(C3)C4)c(Cl)n2cc1NC(=O)c1cccc(C(F)F)n1] |
| 576 | 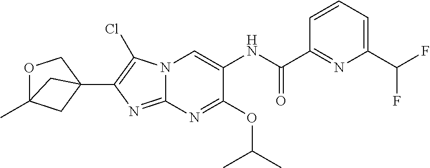 [SMILES: CC(C)Oc1nc2nc(C34COC(C)(C3)C4)c(Cl)n2cc1NC(=O)c1cccc(C(F)F)n1] |
| and 577 | 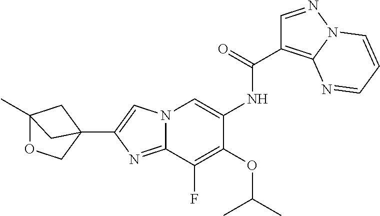 [SMILES: CC(C)Oc1c(NC(=O)c2cnn3cccnc23)cn2cc(C34COC(C)(C3)C4)nc2c1F] |

or a pharmaceutically acceptable salt thereof.

A forty-fourth embodiment of the invention provides a pharmaceutical composition comprising a compound according to any one of the preceding embodiments, or a pharmaceutically acceptable salt thereof.

A forty-fifth embodiment of the invention provides a pharmaceutical composition according to the forty-fourth embodiment, or a pharmaceutically acceptable salt thereof and one or more pharmaceutically acceptable carriers, or diluents.

A forty-sixth embodiment of the invention provides a pharmaceutical composition according to the forty-fifth embodiment, further comprising one or more additional pharmaceutical agent(s).

One embodiment of the invention includes a method of decreasing the expression or activity of IRAK4, or to otherwise affect the properties and/or behavior of IRAK4 polypeptides or polynucleotides comprising administering to said mammal an effective amount of at least one compound described herein, or a pharmaceutically acceptable salt thereof.

A forty-seventh embodiment of the invention is a method of treating an IRAK4 mediated disease in a subject comprising administering to the subject a compound or a pharmaceutically acceptable salt thereof of any one of embodiments one to forty-three or a pharmaceutical composition thereof of any one of embodiments forty-four to forty-six.

A forty-eighth embodiment, the invention provides the use of a compound or a pharmaceutically acceptable salt thereof according to any one of embodiments one to forty-three, or a pharmaceutical composition thereof of any one of embodiments forty-four to forty-six for the treatment of a disorder or disease in a subject mediated by IRAK4.

A forty-ninth embodiment, the invention provides the use of a compound or a pharmaceutically acceptable salt thereof according to any one of embodiments one to forty-three or a pharmaceutical composition thereof of any one of embodiments forty-four to forty-six in manufacture of a medicament for the treatment of a disorder or disease in a subject mediated by IRAK4.

A fifty embodiment of the invention comprising a method of treatment according to embodiment forty-seven, wherein the IRAK4 mediated disease is selected from an autoimmune disease, an inflammatory disease, bone diseases, metabolic diseases, neurological and neurodegenerative diseases and/or disorders, cancer, cardiovascular diseases, allergies, asthma, Alzheimer's disease, hormone-related diseases, Ischemic stroke, Cerebral Ischemia, hypoxia, TBI (Traumatic Brain Injury), CTE (Chronic Traumatic Encephalopathy), epilepsy, multiple sclerosis (MS), Parkinson's disease (PD), and Amyotrophic Lateral Sclerosis (ALS).

A fifty-first embodiment of the invention comprising a method of treatment according to embodiment forty-seven, wherein the IRAK4 mediated disease is selected from disorders and/or conditions associated with inflammation and pain, proliferative diseases, hematopoietic disorders, hematological malignancies, bone disorders, fibrosis diseases and/or disorders, metabolic disorders, muscle diseases and/or disorders, respiratory diseases, pulmonary disorders, genetic development diseases, chronic inflammatory demyelinating neuropathies, vascular or heart diseases, ophthalmic diseases and ocular diseases.

A fifty-second embodiment of the invention comprising a use of a compound according to embodiment forty-eight, wherein the IRAK4 mediated disease is selected from an autoimmune disease, an inflammatory disease, bone diseases, metabolic diseases, neurological and neurodegenerative diseases and/or disorders, cancer, cardiovascular diseases, allergies, asthma, Alzheimer's disease, hormone-related diseases, Ischemic stroke, Cerebral Ischemia, hypoxia, TBI (Traumatic Brain Injury), CTE (Chronic Traumatic Encephalopathy), epilepsy, multiple sclerosis (MS), Parkinson's disease (PD), and Amyotrophic Lateral Sclerosis (ALS).

A fifty-third embodiment of the invention comprising a use of a compound according to embodiment forty-eight, wherein the IRAK4 mediated disease is selected from disorders and/or conditions associated with inflammation and pain, proliferative diseases, hematopoietic disorders, hematological malignancies, bone disorders, fibrosis diseases and/or disorders, metabolic disorders, muscle diseases and/or disorders, respiratory diseases, pulmonary disorders, genetic development diseases, chronic inflammatory demyelinating neuropathies, vascular or heart diseases ophthalmic diseases and ocular diseases.

The compounds, or pharmaceutically acceptable salts thereof described herein may be used to decrease the expression or activity of IRAK4, or to otherwise affect the properties and/or behavior of IRAK4 polypeptides or polynucleotides, e.g., stability, phosphorylation, kinase activity, interactions with other proteins, etc.

One embodiment of the invention includes a method of decreasing the expression or activity of IRAK1, or to otherwise affect the properties and/or behavior of IRAK1 polypeptides or polynucleotides comprising administering to said mammal an effective amount of at least one compound described herein, or a pharmaceutically acceptable salt thereof.

In one embodiment, R¹ is elected from the group consisting of:

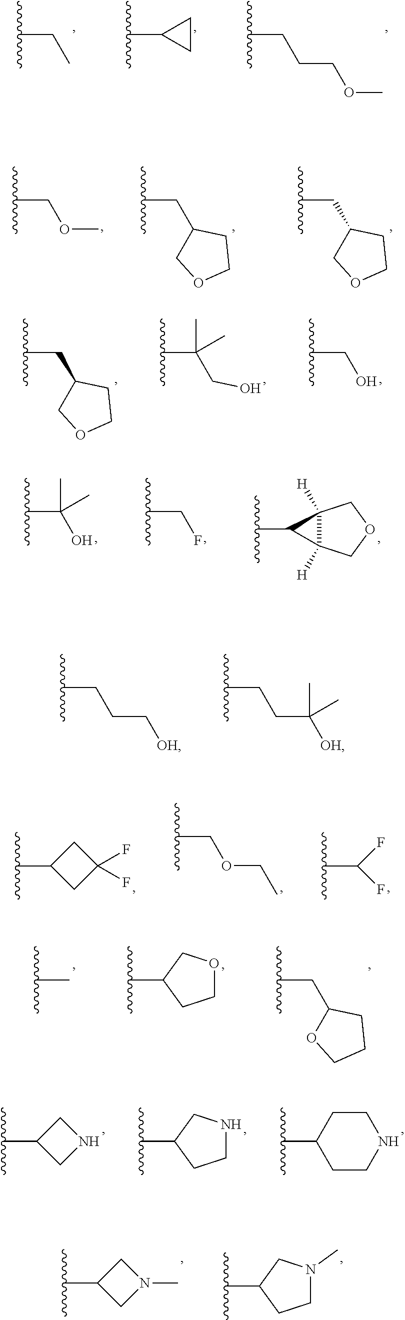

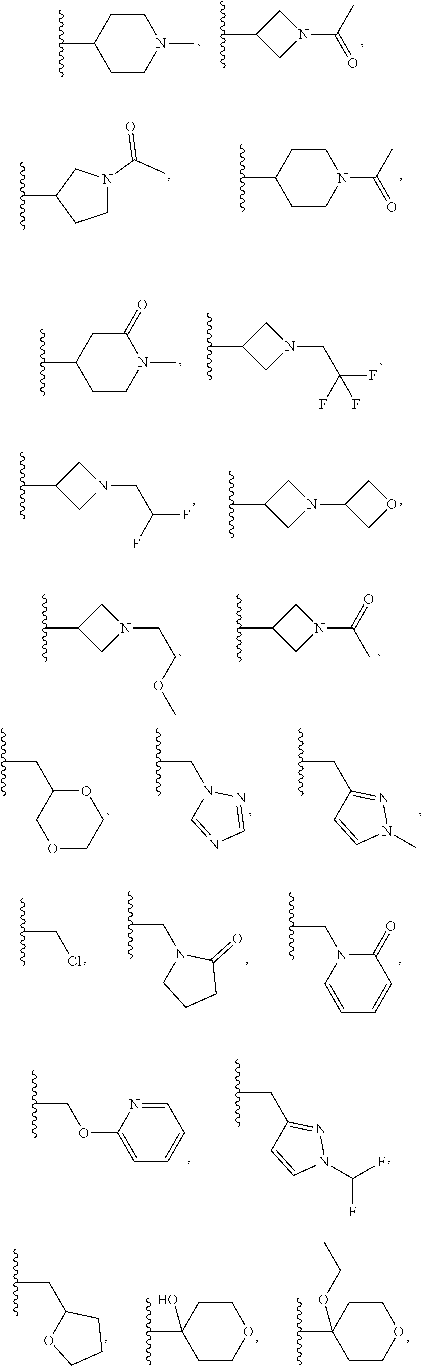

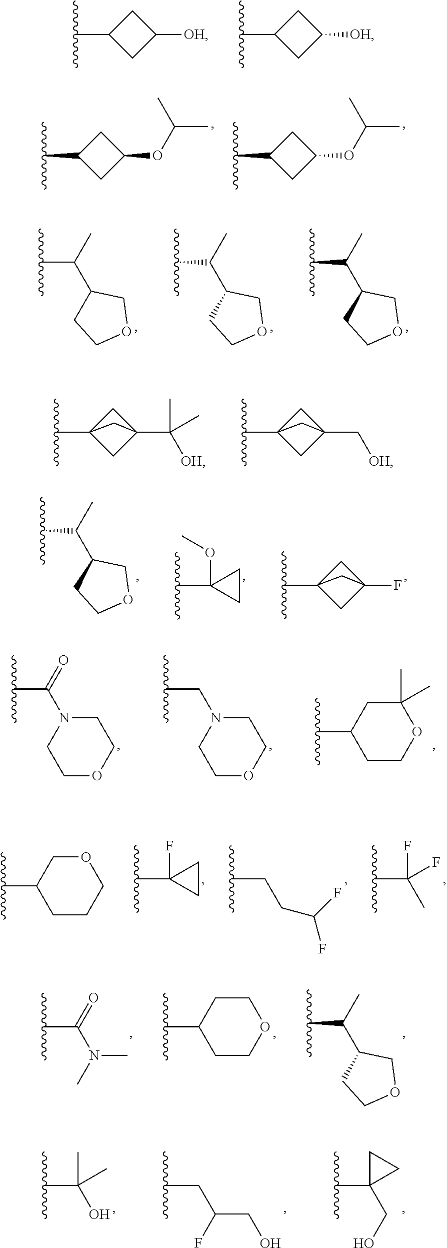

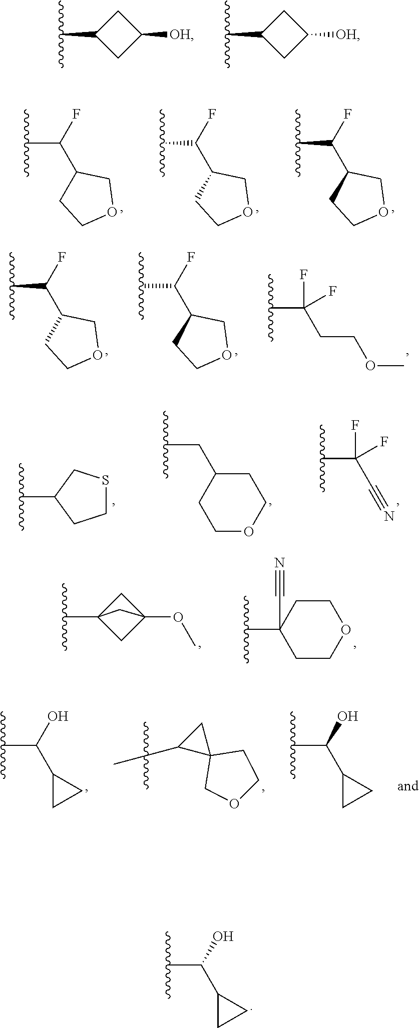

In one embodiment, R¹ is selected from the group consisting of:

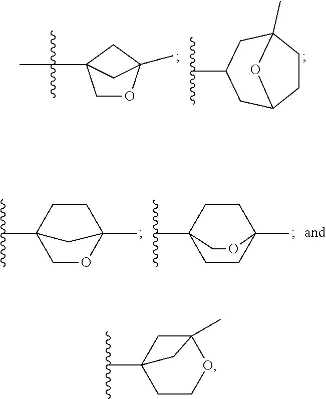

In one embodiment, R³ is elected from the group consisting of

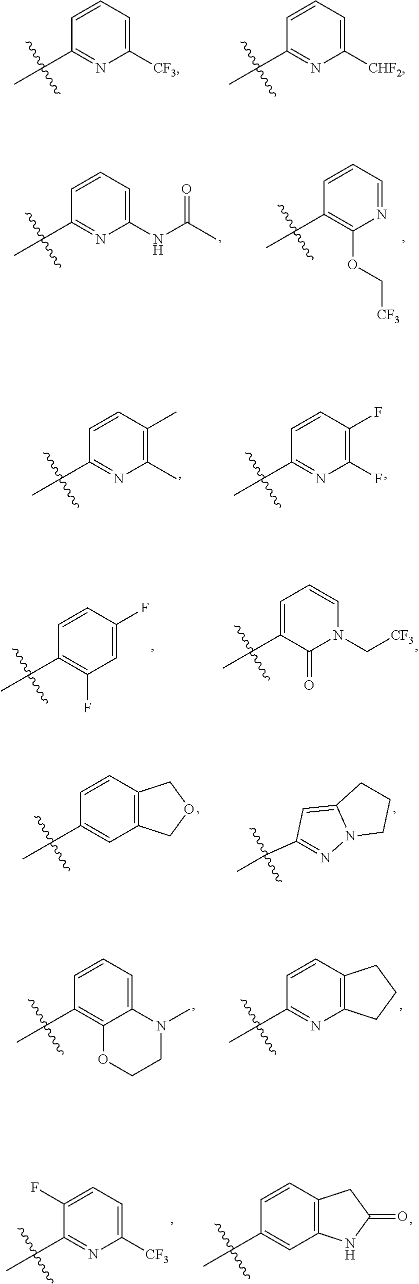

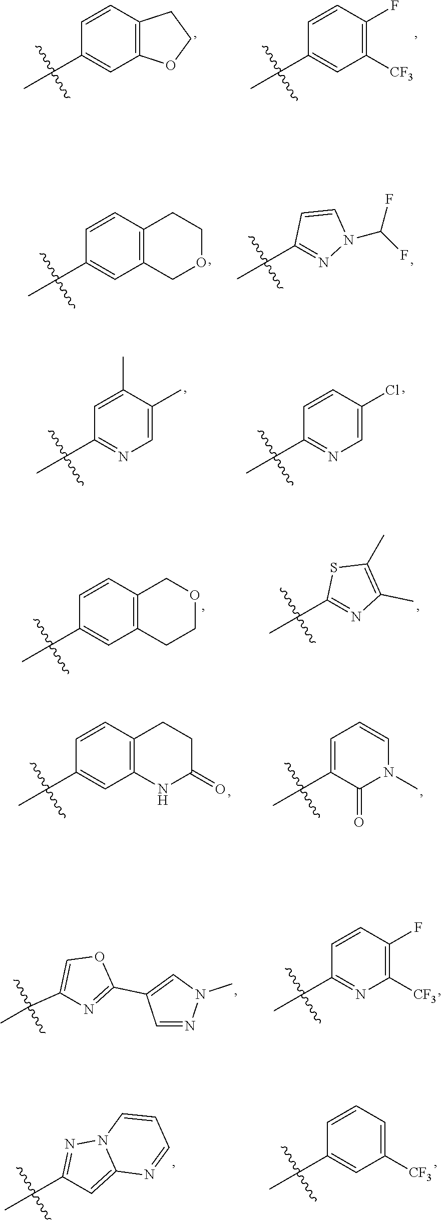

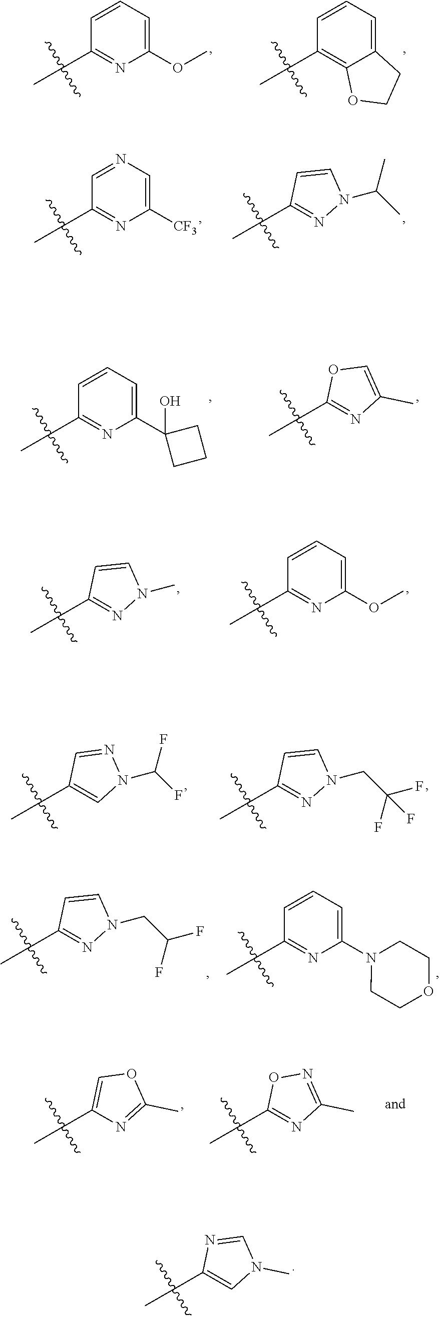

In one embodiment, R³ is selected from the group consisting of:

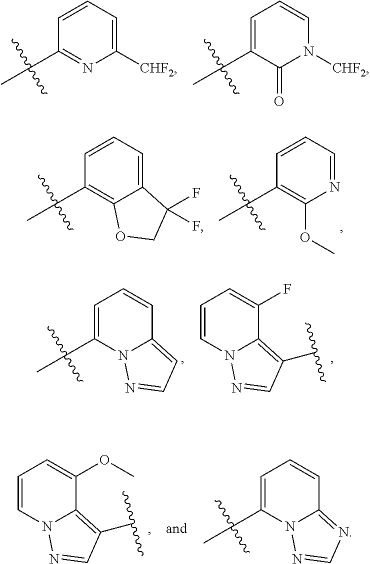

In one embodiment, R⁸ is elected from the group consisting of

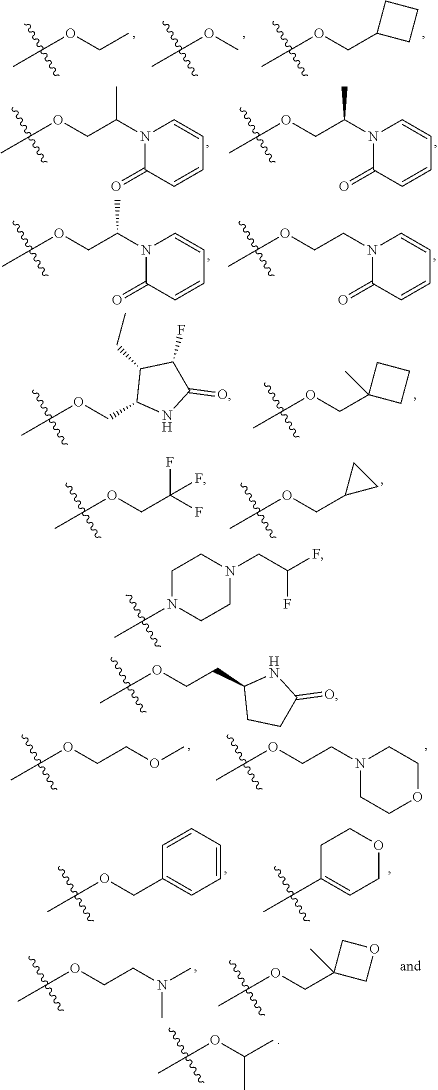

One embodiment of the invention includes a method of decreasing the expression or activity of IRAK4, or to otherwise affect the properties and/or behavior of IRAK4 polypeptides or polynucleotides comprising administering to said subject an effective amount of at least one compound described herein, or a pharmaceutically acceptable salt thereof.

One embodiment of the invention includes a method for treating an inflammatory disease in a subject, the method comprising administering to the patient a therapeutically effective amount of a compound described herein, or a pharmaceutically acceptable salt thereof, thereby treating the inflammatory disease in the subject.

In one embodiment, the inflammatory disease is a pulmonary disease or a disease of the airway.

In one embodiment, the pulmonary disease and disease of the airway is selected from Adult Respiratory Disease Syndrome (ARDS), Chronic Obstructive Pulmonary Disease (COPD), pulmonary fibrosis, interstitial lung disease, asthma, chronic cough, and allergic rhinitis.

In one embodiment, the inflammatory disease is selected from transplant rejection, CD14 mediated sepsis, non-CD14 mediated sepsis, inflammatory bowel disease, Behcet's syndrome, ankylosing spondylitis, sarcoidosis, and gout.

One embodiment of the invention includes a method for treating an autoimmune disease, cancer, cardiovascular disease, a disease of the central nervous system, a disease of the skin, an ophthalmic disease and condition, and bone disease in a subject, the method comprising administering to the patient a therapeutically effective amount of a compound disclosed herein, or a pharmaceutically acceptable salt thereof, thereby treating the autoimmune disease, cancer, cardiovascular disease, disease of the central nervous system, disease of the skin, ophthalmic disease and condition, and bone disease in the subject.

In one embodiment, the autoimmune disease is selected from rheumatoid arthritis, systemic lupus erythematosus, multiple sclerosis, diabetes, systemic sclerosis, and Sjogren's syndrome.

In one embodiment, the autoimmune disease is type 1 diabetes.

In one embodiment, the autoimmune disease is multiple sclerosis.

In one embodiment, the autoimmune disease is epilepsy.

In one embodiment, the cancer is selected from Waldenstrim's macroglobulinemia, solid tumors, skin cancer, and lymphoma.

In one embodiment, the cardiovascular disease is selected from stroke and atherosclerosis.

In one embodiment, the disease of the central nervous system is a neurodegenerative disease.

In one embodiment, the disease of the skin is selected from rash, contact dermatitis, psoriasis, and atopic dermatitis.

In one embodiment, the bone disease is selected from osteoporosis and osteoarthritis.

In one embodiment, the inflammatory bowel disease is selected from Crohn's disease and ulcerative colitis.

One embodiment of the invention includes a method for treating an ischemic fibrotic disease, the method comprising administering to the patient a therapeutically effective amount of a compound described herein, or a pharmaceutically acceptable salt thereof, thereby treating the ischemic fibrotic disease in the subject. In one embodiment, the ischemic fibrotic disease is selected from stroke, acute lung injury, acute kidney injury, ischemic cardiac injury, acute liver injury, and ischemic skeletal muscle injury.

One embodiment of the invention includes a method for treating post-organ transplantation fibrosis, the method comprising administering to the patient a therapeutically effective amount of a compound described herein, or a pharmaceutically acceptable salt thereof, thereby treating post-organ transplantation fibrosis in the subject.

One embodiment of the invention includes a method for treating hypertensive or diabetic end organ disease, the method comprising administering to the patient a therapeutically effective amount of a compound described herein, or a pharmaceutically acceptable salt thereof, thereby treating hypertensive or diabetic end organ disease in the subject.

One embodiment of the invention includes a method for treating hypertensive kidney disease, the method comprising administering to the patient a therapeutically effective amount of a compound described herein, or a pharmaceutically acceptable salt thereof, thereby treating hypertensive kidney disease in the subject.

One embodiment of the invention includes a method for treating idiopathic pulmonary fibrosis (IPF), the method comprising administering to the patient a therapeutically effective amount of a compound described herein, or a pharmaceutically acceptable salt thereof, thereby treating IPF in the subject.

One embodiment of the invention includes a method for treating scleroderma or systemic sclerosis, the method comprising administering to the patient a therapeutically effective amount of a compound described herein, or a pharmaceutically acceptable salt thereof, thereby treating scleroderma or systemic sclerosis in the subject.

One embodiment of the invention includes a method for treating liver cirrhosis, the method comprising administering to the patient a therapeutically effective amount of a compound described herein, or a pharmaceutically acceptable salt thereof, thereby treating liver cirrhosis in the subject.

One embodiment of the invention includes a method for treating fibrotic diseases wherein tissue injury and/or inflammation are present, the method comprising administering to the patient a therapeutically effective amount of a compound described herein, or a pharmaceutically acceptable salt thereof, thereby treating fibrotic diseases where tissue injury and/or inflammation are present in the subject. The fibrotic diseases include, for example, pancreatitis, peritonitis, burns, glomerulonephritis, complications of drug toxicity, and scarring following infections.

Scarring of the internal organs is a major global health problem, which is the consequence of subclinical injury to the organ over a period of time or as the sequela of acute severe injury or inflammation. All organs may be affected by scarring and currently there are few therapies the specifically target the evolution of scarring. Increasing evidence indicates that scarring per se provokes further decline in organ function, inflammation and tissue ischemia. This may be directly due the deposition of the fibrotic matrix which impairs function such as in contractility and relaxation of the heart and vasculature or impaired inflation and deflation of lungs, or by increasing the space between microvasculature and vital cells of the organ that are deprived of nutrients and distorting normal tissue architecture. However recent studies have shown that myofibroblasts themselves are inflammatory cells, generating cytokines, chemokines and radicals that promote injury; and myofibroblasts appear as a result of a transition from cells that normally nurse and maintain the microvasculature, known as pericytes. The consequence of this transition of phenotype is an unstable microvasculature that leads to aberrant angiogenesis, or rarefaction.

The present disclosure relates to methods and compositions for treating, preventing, and/or reducing scarring in organs. More particularly, the present disclosure relates to methods and composition for treating, preventing, and/or reducing scarring in kidneys.

It is contemplated that the present disclosure, methods and compositions described herein can be used as an antifibrotic, or used to treat, prevent, and/or reduce the severity and damage from fibrosis.

It is additionally contemplated that the present disclosure, methods and compositions described herein can be used to treat, prevent, and/or reduce the severity and damage from fibrosis.

It is further contemplated that the present disclosure, methods and compositions described herein can used as an anti-inflammatory, used to treat inflammation.

Some non-limiting examples of organs include: kidney, hearts, lungs, stomach, liver, pancreas, hypothalamus, stomach, uterus, bladder, diaphragm, pancreas, intestines, colon, and so forth.

In certain embodiments, the present invention relates to the aforementioned methods, wherein said compound is administered parenterally.

In certain embodiments, the present invention relates to the aforementioned methods, wherein said compound is administered intramuscularly, intravenously, subcutaneously, orally, pulmonary, rectally, intrathecally, topically or intranasally.

In certain embodiments, the present invention relates to the aforementioned methods, wherein said compound is administered systemically.

In certain embodiments, the present invention relates to the aforementioned methods, wherein said subject is a mammal.

In certain embodiments, the present invention relates to the aforementioned methods, wherein said subject is a primate.

In certain embodiments, the present invention relates to the aforementioned methods, wherein said subject is a human.

The compounds and intermediates described herein may be isolated and used as the compound per se. Alternatively, when a moiety is present that is capable of forming a salt, the compound or intermediate may be isolated and used as its corresponding salt. As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Isotopically-labeled compounds of formula (I′) or (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

It will be recognized by those skilled in the art that the compounds of the present invention may contain chiral centers and as such may exist in different stereoisomeric forms. As used herein, the term “an optical isomer” or “a stereoisomer” refers to any of the various stereo isomeric configurations which may exist for a given compound of the present invention. It is understood that a substituent may be attached at a chiral center of a carbon atom. Therefore, the invention includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term is used to designate a racemic mixture where appropriate. When designating the stereochemistry for the compounds of the present invention, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g, (1RS,2RS) as a racemic mixture of (1R,2R) and (1S,2S); (1RS,2SR) as a racemic mixture of (1R,2S) and (1S,2R)). “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Unless specified otherwise, the compounds of the present invention are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or HPLC chromatography columns, such as CHIRALPAKR™ and CHIRALCEL® available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

### PHARMACOLOGY AND UTILITY

Compounds of the present invention have been found to modulate IRAK4 activity and may be beneficial for the treatment of neurological, neurodegenerative and other additional diseases.

Another aspect of the invention provides a method for treating or lessening the severity of a disease, disorder, or condition associated with the modulation of IRAK4 in a subject, which comprises administering to the subject a compound of Formula (I′) or (I) or a pharmaceutically acceptable salt thereof.

In certain embodiments, the present invention provides a method of treating a condition, disease or disorder implicated by a deficiency of IRAK4 activity, the method comprising administering a composition comprising a compound of formula (I′) or (I) to a subject, preferably a mammal, in need of treatment thereof.

According to the invention an “effective dose” or an “effective amount” of the compound or pharmaceutical composition is that amount effective for treating or lessening the severity of one or more of the diseases, disorders or conditions as recited above.

The compounds and compositions, according to the methods of the present invention, may be administered using any amount and any route of administration effective for treating or lessening the severity of one or more of the diseases, disorders or conditions recited above.

The compounds of the present invention are typically used as a pharmaceutical composition (e.g., a compound of the present invention and at least one pharmaceutically acceptable carrier). As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this invention, solvates and hydrates are considered pharmaceutical compositions comprising a compound of the present invention and a solvent (i.e., solvate) or water (i.e., hydrate).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present invention is generally formulated for use as a parenteral or oral administration.

For example, the pharmaceutical oral compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with

a) diluents, e.g., lactose, dextrose, sucrose, mannitol, sorbitol, cellulose and/or glycine;

b) lubricants, e.g., silica, talcum, stearic acid, its magnesium or calcium salt and/or polyethyleneglycol; for tablets also

c) binders, e.g., magnesium aluminum silicate, starch paste, gelatin, tragacanth, methylcellulose, sodium carboxymethylcellulose and/or polyvinylpyrrolidone; if desired

d) disintegrants, e.g., starches, agar, alginic acid or its sodium salt, or effervescent mixtures; and/or

e) absorbents, colorants, flavors and sweeteners.

Tablets may be either film coated or enteric coated according to methods known in the art.

Suitable compositions for oral administration include a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present invention or pharmaceutical composition thereof for use in a subject (e.g., human) is typically administered orally or parenterally at a therapeutic dose of less than or equal to about 100 mg/kg, 75 mg/kg, 50 mg/kg, 25 mg/kg, 10 mg/kg, 7.5 mg/kg, 5.0 mg/kg, 3.0 mg/kg, 1.0 mg/kg, 0.5 mg/kg, 0.05 mg/kg or 0.01 mg/kg, but preferably not less than about 0.0001 mg/kg. When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations.

### Combination Therapy

The compounds of the present invention can be used, alone or in combination with other therapeutic agents, in the treatment of various conditions or disease states. The compound(s) of the present invention and other therapeutic agent(s) may be administered simultaneously (either in the same dosage form or in separate dosage forms) or sequentially.

Two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

The present invention includes the use of a combination of an IRAK inhibitor compound as provided in the compound of formula (I′) or (I) and one or more additional pharmaceutically active agent(s). If a combination of active agents is administered, then they may be administered sequentially or simultaneously, in separate dosage forms or combined in a single dosage form. Accordingly, the present invention also includes pharmaceutical compositions comprising an amount of: (a) a first agent comprising a compound of formula (I′) or (I) or a pharmaceutically acceptable salt of the compound; (b) a second pharmaceutically active agent; and (c) a pharmaceutically acceptable carrier, vehicle or diluent.

The compounds of the present invention can be administered alone or in combination with one or more additional therapeutic agents. By “administered in combination” or “combination therapy” it is meant that a compound of the present invention and one or more additional therapeutic agents are administered concurrently to the mammal being treated. When administered in combination each component may be administered at the same time or sequentially in any order at different points in time. Thus, each component may be administered separately but sufficiently closely in time to provide the desired therapeutic effect. Thus, the methods of prevention and treatment described herein include use of combination agents.

The combination agents are administered to a mammal, including a human, in a therapeutically effective amount. By “therapeutically effective amount” it is meant an amount of a compound of the present invention that, when administered alone or in combination with an additional therapeutic agent to a mammal, is effective to treat the desired disease/condition e.g., inflammatory condition such as systemic lupus erythematosus. See also, T. Koutsokeras and T. Healy, Systemic lupus erythematosus and lupus nephritis, Nat Rev Drug Discov, 2014, 13(3), 173-174, for therapeutic agents useful treating lupus.

In particular, it is contemplated that the compounds of the invention may be administered with the following therapeutic agents: Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, T F blockers, IL-1 RA, azathioprine, cyclophosphamide, and sulfasalazine; immunomodulatory and immunosuppressive agents such as cyclosporin, tacrolimus, rapamycin, rnycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents: agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3 A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

### Definitions

As used herein, a “patient,” “subject” or “individual” are used interchangeably and refer to either a human or non-human animal. The term includes mammals such as humans. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder, refers to the management and care of a patient for the purpose of combating the disease, condition, or disorder and includes the administration of a compound of the present invention to prevent the onset of the symptoms or complications, alleviating the symptoms or complications, or eliminating the disease, condition or disorder.

As used herein the term “stroke” has the meaning normally accepted in the art. The term can broadly refer to the development of neurological deficits associated with the impaired blood flow regardless of cause. Potential causes include, but are not limited to, thrombosis, hemorrhage and embolism. The term “ischemic stroke” refers more specifically to a type of stroke that is of limited extent and caused due to a blockage of blood flow.

As used herein, a subject is “in need of” a treatment if such subject would benefit biologically, medically or in quality of life from such treatment (preferably, a human).

As used herein the term “co-administer” refers to the presence of two active agents in the blood of an individual. Active agents that are co-administered can be concurrently or sequentially delivered.

The term “combination therapy” or “in combination with” or “pharmaceutical combination” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent being administered prior to, concurrent with, or sequentially to each other with no specific time limits. In each case, the treatment regimen will provide beneficial effects of the drug combination in treating the conditions or disorders described herein.

As used herein, the phrase “optionally substituted” is used interchangeably with the phrase “substituted or unsubstituted.” In general, the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position.

As used herein, the term “C₁₋₅alkyl” refers to a fully saturated branched or unbranched hydrocarbon moiety having 1 to 5 carbon atoms. The terms “C₁₋₄alkyl”, “C₁₋₃alkyl” and “C₁₋₂alkyl” are to be construed accordingly. Representative examples of “C₁₋₅alkyl” include, but are not limited to, methyl, ethyl, n-propyl, iso-propyl, n-butyl, sec-butyl, iso-butyl, tert-butyl, n-pentyl, isopentyl and neopentyl. Similarly, the alkyl portion (i.e., alkyl moiety) of an alkoxy have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls). “Halo-substituted alkyl” refers to an alkyl group having at least one halogen substitution.

As used herein, the term “C₁₋₄ alkoxy” refers to a fully saturated branched or unbranched alkyl moiety attached through an oxygen bridge (i.e. a —O— C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. Preferably, alkoxy groups have about 1-4 carbons, more preferably about 1-2 carbons. The term “C₁₋₂ alkoxy” is to be construed accordingly.

“Halogen” or “halo” may be fluorine, chlorine, bromine or iodine (preferred halogens as substituents are fluorine and chlorine).

As used herein, the term “halo-substituted-C₁₋₄alkyl” or “halo-C₁₋₄ alkyl” refers to a C₁₋₄ alkyl group as defined herein, wherein at least one of the hydrogen atoms is replaced by a halo atom. The halo-C₁₋₄alkyl group can be monohalo-C₁₋₄alkyl, dihalo-C₁₋₄alkyl or polyhalo-C₁₋₄ alkyl including perhalo-C₁₋₄alkyl. A monohalo-C₁₋₄alkyl can have one iodo, bromo, chloro or fluoro within the alkyl group. Dihalo-C₁₋₄alkyl and polyhalo-C₁₋₄alkyl groups can have two or more of the same halo atoms or a combination of different halo groups within the alkyl. Typically, the polyhalo-C₁₋₄alkyl group contains up to 9, or 8, or 7, or 6, or 5, or 4, or 3, or 2 halo groups. Non-limiting examples of halo-C₁₋₄alkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-C₁₋₄alkyl group refers to a C₁₋₄alkyl group having all hydrogen atoms replaced with halo atoms.

As used herein, the term “halo-substituted-C₁₋₄alkoxy” or “halo-C₁₋₄alkoxy” refers to C₁₋₄ alkoxy group as defined herein above wherein at least one of the hydrogen atoms is replaced by a halo atom. Non-limiting examples of halo-substituted-C₁₋₄alkoxy include fluoromethoxy, difluoromethoxy, trifluoromethoxy, chloromethoxy, dichloromethoxy, trichloromethoxy, difluorochloromethoxy, dichlorofluoromethoxy, difluoroethoxy, difluoropropoxy, dichloroethoxy and dichloropropoxy and the like.

As used herein “Hydroxyl” or “Hydroxy” refers to the group —OH.

As used herein, the term “hydroxy-substituted-C₁₋₄ alkyl” refers to a C₁₋₄ alkyl group as defined herein, wherein at least one of the hydrogen atoms is replaced by a hydroxyl group. The hydroxy-substituted-C₁₋₄ alkyl group can be monohydroxy-C₁₋₄ alkyl, dihydroxy-C₁₋₄ alkyl or polyhydroxy-C₁₋₄ alkyl including perhydroxy-C₁₋₄ alkyl. A monohydroxy-C₁₋₄ alkyl can have one hydroxyl group within the alkyl group. Dihydroxy-C₁₋₄ alkyl and polyhydroxy-C₁₋₄ alkyl groups can have two or more of the same hydroxyl groups or a combination of different hydroxyl groups within the alkyl. Typically, the polyhydroxy-C1-4alkyl group contains up to 9, or 8, or 7, or 6, or 5, or 4, or 3, or 2 hydroxy groups. Non-limiting examples of hydroxy substituted-C₁₋₄ alkyl include hydroxy-methyl, dihydroxy-methyl, pentahydroxy-ethyl, dihydroxyethyl, and dihydroxypropyl. A perhydroxy-C₁₋₄ alkyl group refers to a C₁₋₄ alkyl group having all hydrogen atoms replaced with hydroxy atoms.

The term “oxo” (═O) refers to an oxygen atom connected to a carbon or sulfur atom by a double bond. Examples include carbonyl, sulfinyl, or sulfonyl groups (—C(O)—, —S(O)— or —S(O)₂—) such as, a ketone, aldehyde, or part of an acid, ester, amide, lactone, or lactam group and the like.

The term “aryl or C₆₋₁₀ aryl” refers to 6- to 10-membered aromatic carbocyclic moieties having a single (e.g., phenyl) or a fused ring system (e.g., naphthalene.). A typical aryl group is phenyl group.

The term “heteroaryl” refers to aromatic ring moieties containing at least one heteroatom (e.g., oxygen, sulfur, nitrogen or combinations thereof) within a 5- to 6-membered aromatic ring system (e.g., pyrrolyl, pyridyl, pyrazolyl, thienyl, furanyl, oxazolyl, imidazolyl, tetrazolyl, triazinyl, pyrimidyl, pyrazinyl, thiazolyl, and the like), or within a 9- to 10-membered fused aromatic ring system (e.g., indolyl, indazolyl, benzofuranyl, quinoxalinyl and the like.)

The term “5 to 6 membered heteroaryl” or “C₅₋₆ heteroaryl” refers to aromatic heterocyclic moieties containing at least one heteroatom (e.g., oxygen, sulfur, nitrogen or combinations thereof) within a 5- to 6-membered aromatic ring system.

The term “9 to 10 membered heteroaryl” or “C₉₋₁₀ heteroaryl” refers to aromatic moieties containing at least one heteroatom (e.g., oxygen, sulfur, nitrogen or combinations thereof) within a 9- to 10-membered fused aromatic ring system.

The term “fully or partially saturated carbocyclic ring” refers to a nonaromatic hydrocarbon ring that is either partially or fully saturated and may exist as a single ring, bicyclic ring (including fused, spiral or bridged carbocyclic rings). Unless specified otherwise, the carbocyclic ring generally contains 4- to 7-ring members.

The term “C₃₋₆ cycloalkyl” refers to a fully saturated carbocyclic ring containing 3- to 6-ring carbon atoms. Examples of C₃₋₆ cycloalkyl include, but are not limited to, cyclopropyl, cyclobutyl, cyclopentyl, and cyclohexyl.

The term “C₃₋₆ cycloalkoxy” refers to —O—C₃₋₆ cycloalkyl moiety, in which the cycloalkyl moiety is attached through an oxygen bridge, wherein C₃₋₆ cycloalkyl is as defined herein.

The term “fully or partially saturated heterocycle” or “fully or partially saturated 4 to 7 membered heterocycle” refers to a nonaromatic ring that is either partially or fully saturated and may exist as a single ring, bicyclic ring (including fused heterocyclic rings) or a spiral ring. Unless specified otherwise, the heterocyclic ring is generally a 4 to 7-membered ring containing 1 to 3 heteroatoms (preferably 1, 2 or 3 heteroatoms) independently selected from sulfur, oxygen and/or nitrogen. A partially saturated heterocyclic ring also includes groups wherein the heterocyclic ring is fused to an aryl or heteroaryl ring (e.g., 2,3-dihydrobenzofuranyl, indolinyl (or 2,3-dihydroindolyl), 2,3-dihydrobenzothiophenyl, 2,3-dihydrobenzothiazolyl, 1,2,3,4-tetrahydroquinolinyl, 1,2,3,4-tetrahydroisoquinolinyl, 5,6,7,8-tetrahydropyrido[3,4-b]pyrazinyl). Partially saturated or fully saturated heterocyclic rings include groups such as epoxy, aziridinyl, azetidinyl, tetrahydrofuranyl, dihydrofuranyl, dihydropyridinyl, pyrrolidinyl, imidazolidinyl, imidazolinyl, 1H-dihydroimidazolyl, hexahydropyrimidinyl, piperidinyl, piperazinyl, pyrazolidinyl, 2H-pyranyl, 4H-pyranyl, oxazinyl, morpholino, thiomorpholino, tetrahydrothienyl, tetrahydrothienyl 1,1-dioxide, oxazolidinyl, thiazolidinyl, 7-oxabicyclo[2.2.1]heptane, and the like. In certain embodiment, the “fully or partially saturated 4 to 7 membered heterocycle” or “C₄₋₇ heterocycle” refers to a fully or partially saturated monocyclic ring containing 4 to 7 ring atoms, which includes 1 to 2 heteroatoms independently selected from sulfur, oxygen and/or nitrogen. A typical “C₄₋₇ heterocycle” group includes oxtanyl, tetrahydrofuranyl, dihydrofuranyl, 1,4-dioxanyl, morpholinyl, 1,4-dithianyl, piperazinyl, piperidinyl, 1,3-dioxolanyl, pyrrolinyl, pyrrolidinyl, tetrahydropyranyl, oxathiolanyl, dithiolanyl, 1,3-dioxanyl, 1,3-dithianyl, oxathianyl, thiomorpholinyl, thiomorpholinyl 1,1 dioxide, tetrahydro-thiopyran 1,1-dioxide, 1,4-diazepanyl.

The term “spiro ring system,” as used herein, is a ring system that has two rings each of which are independently selected from a carbocyclic ring or a heterocyclic ring, wherein the two ring structures having one ring atom in common. In one embodiment, spiro ring systems have from 5 to 10 ring atoms.

The term “fused ring system”, as used herein, is a ring system that has two rings each of which are independently selected from a carbocyclic ring or a heterocyclic ring, wherein the two ring structures share two adjacent ring atoms. In one embodiment, the fused ring system is a fused bicyclic ring system including 8 to 10 ring atoms. An 8 to 10 membered fused bicyclic ring system can be a non-aromatic ring system or an aromatic ring system (e.g., aryl and 9 to 10 membered heteroaryl as defined herein). In some embodiment, the 8 to 10 membered fused bicyclic ring system optionally includes 1, 2, or 3 heteroatoms independently selected from oxygen, nitrogen, and sulfur.

The term “bridged ring system”, as used herein, is a ring system that has a carbocyclic or heterocyclic ring wherein two non-adjacent atoms of the ring are connected (bridged) by one or more (preferably from one to three) atoms independently selected from C, N, O, and S. In one embodiment, a bridged ring system has 5 to 10 ring atoms.

As used herein the term “5 to 10 membered spiro heterobicyclic ring system” means a two-ring system containing 5 to 10 ring atoms, at least one of which is a heteroatom selected from O, N, and S, wherein the two rings share one common atom. Examples of spiro rings include oxaspiro[2.4]heptane, 2,6-diazaspiro[3.3]heptanyl, -oxa-6-azaspiro[3.3]heptane, 2,2,6-diazaspiro[3.3]heptane, 3-azaspiro[5.5]undecanyl, 3,9-diazaspiro[5.5]undecanyl, 7-azaspiro[3.5]nonane, 2,6-diazaspiro[3.4]octane, 8-azaspiro[4.5]decane, 1,6-diazaspiro[3.3]heptane, 5-azaspiro[2.5]octane, 4,7-diazaspiro[2.5]octane, 5-oxa-2-azaspiro[3.4]octane, 6-oxa-1-azaspiro[3.3]heptane, 3-azaspiro[5.5]undecanyl, 3,9-diazaspiro[5.5]undecanyl, and the like.

As used herein the term “3 to 8 membered spirocycloalkyl” means a two-cycloalkyl-ring system containing 3 to 8 ring atoms, wherein the two rings share one common carbon atom. Examples of spiro 3-8 membered cycloalkyl rings include spiro[2.5]octane, spiro[2.4]heptane, spiro[3.4]octane and the like. The term “fused heterocycle” or “8 to 10 membered fused heterobicyclic ring system” refers to two ring system containing 8- to 10-ring atoms, at least one of which is a heteroatom selected from O, N, and S, wherein the two rings share two adjacent ring atoms. Examples of fused heterocycles include fully or partially saturated groups such as 1,3-dihydroisobenzofuran, 4-methyl-3,4-dihydro-2H-benzo[b][1,4]oxazine, pyrazolo[1,5-a]pyrimidine, 5,6-dihydro-4H-pyrrolo[1,2-b]pyrazole, 6,7-dihydro-5H-cyclopenta[b]pyridine, indolin-2-one, 2,3-dihydrobenzofuran, 1-methyl-2-oxo-1,2,3,4-tetrahydroquinoline, 3,4-dihydroquinolin-2(1H)-one, and isochromane, 4,5,6,7-tetrahydro-3H-imidazo[4,5-c]pyridine, 8-azabicyclo[3.2.1]octan-3-ol, octahydropyrrolo[1,2-a]pyrazine, 5,6,7,8-tetrahydroimidazo[1,2-a]pyrazine, 3,8 diazabicyclo[3.2.1]octane, 8-oxa-3-azabicyclo[3.2.1]octane, 7-oxabicyclo[2.2.1]heptane, 1H-pyrazole, 2,5-diazabicyclo[2.2.1]heptane, 5,6,7,8-tetrahydro-[1,2,4]triazolo[4,3-a]pyrazine or 3-azabicyclo[3.1.0]hexane. A partially saturated heterocyclic ring also includes groups wherein the heterocyclic ring is fused to an aryl or heteroaryl ring (e.g., 2,3-dihydrobenzofuranyl, indolinyl (or 2,3-dihydroindolyl), 2,3-dihydrobenzothiophenyl, 2,3-dihydrobenzothiazolyl, 1,2,3,4-tetrahydroquinolinyl, 1,2,3,4-tetrahydroisoquinolinyl, 5,6,7,8-tetrahydropyrido[3,4-b]pyrazinyl, and the like).

As used herein the term “bridged-carbocyclic ring” or “5 to 10 membered bridged-carbocyclic ring” refers to a fully or partially saturated 5 to 10 membered carbocyclic ring system, wherein two non-adjacent atoms of the ring are connect (bridged) by one or more C atoms, Examples of bridged-carbocyclic ring include bicyclo[1.1.1]pentane, bicyclo [2.2.1]heptane and bicyclo [3.2.1] octane.

As used herein the term “bridged-heterocyclic ring” or “5 to 10 membered bridged-heterocyclic ring” refers to a 5 to 10 membered heterobicyclic ring system containing at least one heteroatom (e.g., oxygen, sulfur, nitrogen or combinations thereof), wherein two non-adjacent atoms of the ring are connected (bridged) by one or more (preferably from one to three) atoms selected from C, N, O, and S.

The phrase “pharmaceutically acceptable” indicates that the substance, composition or dosage form must be compatible chemically and/or toxicologically, with the other ingredients comprising a formulation, and/or the mammal being treated therewith.

Unless specified otherwise, the term “compounds of the present invention” refers to compounds of formula (I′) or (I), as well as all stereoisomers (including diastereoisomers and enantiomers), rotamers, tautomers, isotopically labeled compounds (including deuterium substitutions), and inherently formed moieties (e.g., polymorphs, solvates and/or hydrates). When a moiety is present that is capable of forming a salt, then salts are included as well, in particular pharmaceutically acceptable salts.

As used herein, the term “a,” “an,” “the” and similar terms used in the context of the present invention (especially in the context of the claims) are to be construed to cover both the singular and plural unless otherwise indicated herein or clearly contradicted by the context. The use of any and all examples, or exemplary language (e.g. “such as”) provided herein is intended merely to better illuminate the invention and does not pose a limitation on the scope of the invention otherwise claimed.

In one Embodiment, there is provided a compound of the Examples as an isolated stereoisomer wherein the compound has one stereocenter and the stereoisomer is in the R configuration.

In one Embodiment, there is provided a compound of the Examples as an isolated stereoisomer wherein the compound has one stereocenter and the stereoisomer is in the S configuration.

In one Embodiment, there is provided a compound of the Examples as an isolated stereoisomer wherein the compound has two stereocenters and the stereoisomer is in the R R configuration.

In one Embodiment, there is provided a compound of the Examples as an isolated stereoisomer wherein the compound has two stereocenters and the stereoisomer is in the R S configuration.

In one Embodiment, there is provided a compound of the Examples as an isolated stereoisomer wherein the compound has two stereocenters and the stereoisomer is in the S R configuration.

In one Embodiment, there is provided a compound of the Examples as an isolated stereoisomer wherein the compound has two stereocenters and the stereoisomer is in the S S configuration.

In one Embodiment, there is provided a compound of the Examples, wherein the compound has one or two stereocenters, as a racemic mixture.

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

In one Embodiment, the invention relates to a compound of the formula (I′) or (I) as defined herein, in free form. In another Embodiment, the invention relates to a compound of the formula (I′) or (I) as defined herein, in salt form. In another Embodiment, the invention relates to a compound of the formula (I′) or (I) as defined herein, in acid addition salt form. In a further Embodiment, the invention relates to a compound of the formula (I′) or (I) as defined herein, in pharmaceutically acceptable salt form. In yet a further Embodiment, the invention relates to a compound of the formula (I′) or (I) as defined herein, in pharmaceutically acceptable acid addition salt form. In yet a further Embodiment, the invention relates to any one of the compounds of the Examples in free form. In yet a further Embodiment, the invention relates to any one of the compounds of the Examples in salt form. In yet a further Embodiment, the invention relates to any one of the compounds of the Examples in acid addition salt form. In yet a further Embodiment, the invention relates to any one of the compounds of the Examples in pharmaceutically acceptable salt form. In still another Embodiment, the invention relates to any one of the compounds of the Examples in pharmaceutically acceptable acid addition salt form.

Furthermore, the compounds of the present invention, including their salts, may also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

Compounds of the invention, i.e. compounds of formula (I′) or (I) that contain groups capable of acting as donors and/or acceptors for hydrogen bonds may be capable of forming co-crystals with suitable co-crystal formers. These co-crystals may be prepared from compounds of formula (I′) or (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I′) or (I).

The compounds of the present invention, including salts, hydrates and solvates thereof, may inherently or by design form polymorphs.

Compounds of the present invention may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

The further optional reduction, oxidation or other functionalization of compounds of formula (I′) or (I) may be carried out according to methods well known to those skilled in the art. Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, and in H.-D. Jakubke and H. Jeschkeit, “Aminosauren, Peptide, Proteine” (Amino acids, Peptides, Proteins), Verlag Chemie, Weinheim, Deerfield Beach, and Basel 1982. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of the present invention having at least one salt-forming group may be prepared in a manner known to those skilled in the art. For example, acid addition salts of compounds of the present invention are obtained in customary manner, e.g. by treating the compounds with an acid or a suitable anion exchange reagent. Salts can be converted into the free compounds in accordance with methods known to those skilled in the art. Acid addition salts can be converted, for example, by treatment with a suitable basic agent.

Any resulting mixtures of isomers can be separated on the basis of the physicochemical differences of the constituents, into the pure or substantially pure geometric or optical isomers, diastereomers, racemates, for example, by chromatography and/or fractional crystallization.

For those compounds containing an asymmetric carbon atom, the compounds exist in individual optically active isomeric forms or as mixtures thereof, e.g. as racemic or diastereomeric mixtures. Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a commercially available chiral HPLC column.

The invention further includes any variant of the present processes, in which the reaction components are used in the form of their salts or optically pure material. Compounds of the invention and intermediates can also be converted into each other according to methods generally known to those skilled in the art.

For illustrative purposes, the reaction schemes depicted below provide potential routes for synthesizing the compounds of the present invention as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### GENERAL METHODS

The compounds of the Examples were analyzed or purified according to one of the Purification Methods referred to below unless otherwise described.

Chromatography on silica gel was carried out using 20-40 μM (particle size), 250-400 mesh, or 400-632 mesh silica gel using either a Teledyne ISCO Combiflash RF or a Grace Reveleris X2 with ELSD purification systems.

### Analytical Methods

ESI-MS data (also reported herein as simply MS) were recorded using Waters System (Acquity HPLC and a Micromass ZQ mass spectrometer); all masses reported are the m/z of the protonated parent ions unless recorded otherwise.

### LC/MS:

The sample is dissolved in a suitable solvent such as MeCN, DMSO or MeOH and is injected directly into the column using an automated sample handler. The analysis using one of the following methods:

The invention further includes any variant of the present processes, in which the reaction components are used in the form of their salts or optically pure material. Compounds of the invention and intermediates can also be converted into each other according to methods generally known to those skilled in the art.

### Analytical HPLC

Acidic HPLC: Conducted on a Shimadza 20A instrument with an ultimate C18 3.0×50 mm, 3 μm column eluting with 2.75 mL/4 L TFA in water (solvent A) and 2.5 mL/4 L TFA in acetonitrile (solvent B) by the following methods:

Method A: using the following elution gradient 0%-60% (solvent B) over 6 minutes and holding at 60% for 2 minutes at a flow rate of 1.2 ml/minutes. Wavelength: UV 220 nm, 215 nm and 254 nm.

Method B: using the following elution gradient 10%-80% (solvent B) over 6 minutes and holding at 60% for 2 minutes at a flow rate of 1.2 ml/minutes. Wavelength: UV 220 nm, 215 nm and 254 nm.

Method C: using the following elution gradient 30%-90% (solvent B) over 6 minutes and holding at 60% for 2 minutes at a flow rate of 1.2 ml/minutes. Wavelength: UV 220 nm, 215 nm and 254 nm.

Basic HPLC: Conducted on a Shimadza 20A instrument with Xbrige Shield RP-18, 5 um, 2.1×50 mm column eluting with 2 mL/4 L NH3H2O in water (solvent A) and acetonitrile (solvent B), by the following methods:

Method D: using the following elution gradient 0%-60% (solvent B) over 4.0 minutes and holding at 60% for 2 minutes at a flow rate of 1.2 ml/minutes.

Method E: using the following elution gradient 10%-80% (solvent B) over 4.0 minutes and holding at 60% for 2 minutes at a flow rate of 1.2 ml/minutes.

Method F: using the following elution gradient 30%-90% (solvent B) over 4.0 minutes and holding at 60% for 2 minutes at a flow rate of 1.2 ml/minutes.

### Analytical LCMS

Acidic LCMS: Conducted on a Shimadza 2010 Series, Shimadza 2020 Series, or Waters Acquity UPLC BEH. (MS ionization: ESI) instrument equipped with a C18 column (2.1 mm×30 mm, 3.0 mm or 2.1 mm×50 mm, C18, 1.7 μm), eluting with 1.5 mL/4 L TFA in water (solvent A) and 0.75 mL/4LTFA in acetonitrile (solvent B) using the methods below:

1.5 Minute Methods:

General method: using the following elution gradient 5%-95% (solvent B) over 0.7 minutes and holding at 95% for 0.4 minutes at a flow rate of 1.5 ml/minutes. Wavelength: UV 220 nm and 254 nm.

2 Minute Methods:

Method A: using the following elution gradient 0%-60% (solvent B) over 0.9 minutes and holding at 60% for 0.6 minutes at a flow rate of 1.2 ml/minutes. Wavelength: UV 220 nm and 254 nm.

Method B: using the following elution gradient 10%-80% (solvent B) over 0.9 minutes and holding at 60% for 0.6 minutes at a flow rate of 1.2 ml/minutes. Wavelength: UV 220 nm and 254 nm.

Method C: using the following elution gradient 30%-90% (solvent B) over 0.9 minutes and holding at 60% for 0.6 minutes at a flow rate of 1.2 ml/minutes. Wavelength: UV 220 nm and 254 nm.

3.5 Minute Method:

Initial conditions, solvent A-95%: solvent B-5%; hold at initial from 0.0-0.1 min; Linear Ramp to solvent A-5%: solvent B-95% between 0.1-3.25 min; hold at solvent A-5%:solvent B-95% between 3.25-3.5 min. Diode array/MS detection.

4 Minute Methods:

Method A: using the following elution gradient 0%-60% (solvent B) over 3 minutes and holding at 60% for 0.5 minutes at a flow rate of 0.8 ml/minutes. Wavelength: UV 220 nm and 254 nm.

Method B: using the following elution gradient 10%-80% (solvent B) over 3 minutes and holding at 60% for 0.5 minutes at a flow rate of 0.8 ml/minutes. Wavelength: UV 220 nm and 254 nm.

Method C: using the following elution gradient 30%-90% (solvent B) over 3 minutes and holding at 60% for 0.5 minutes at a flow rate of 0.8 ml/minutes. Wavelength: UV 220 nm and 254 nm.

7 Minute Methods:

Method A: using the following elution gradient 0%-60% (solvent B) over 6 minutes and holding at 60% for 0.5 minutes at a flow rate of 0.8 ml/minutes. Wavelength: UV 220 nm and 254 nm.

Method B: using the following elution gradient 10%-80% (solvent B) over 6 minutes and holding at 60% for 0.5 minutes at a flow rate of 0.8 ml/minutes. Wavelength: UV 220 nm and 254 nm.

Method C: using the following elution gradient 30%-900% (solvent B) over 6 minutes and holding at 60% for 0.5 minutes at a flow rate of 0.8 ml/minutes. Wavelength: UV 220 nm and 254 nm.

Basic LCMS: Conducted on a Shimadza 2020 Series or Waters Acquity UPLC BEH (MS ionization: ESI) instrument equipped with XBridge Shield RP18, 5 um column (2.1 mm×30 mm, 3.0 mm i.d.) or 2.1 mm×50 mm, C18, 1.7 μm column, eluting with 2 mL/4 L NH3H2O in water (solvent A) and acetonitrile (solvent B) using the methods below:

3 Minute Methods:

Method A: using the following elution gradient 0%-60% (solvent B) over 2 minutes and holding at 60% for 0.48 minutes at a flow rate of 1 ml/minutes. Wavelength: UV 220 nm and 254 nm.

Method B: using the following elution gradient 10%-80% (solvent B) over 2 minutes and holding at 60% for 0.48 minutes at a flow rate of 1 ml/minutes. Wavelength: UV 220 nm and 254 nm.

Method C: using the following elution gradient 30%-90% (solvent B) over 2 minutes and holding at 60% for 0.48 minutes at a flow rate of 1 ml/minutes. Wavelength: UV 220 nm and 254 nm.

3.5 Minute Method:

Initial conditions, solvent A-95%:solvent B-5%; hold at initial from 0.0-0.1 min; Linear Ramp to solvent A-5%:solvent B-95% between 0.1-3.25 min; hold at solvent A-5%:solvent B-95% between 3.25-3.5 min. Diode array/MS detection.

7 Minute Methods:

Method A: using the following elution gradient 0%-60% (solvent B) over 6 minutes and holding at 60% for 0.5 minutes at a flow rate of 0.8 ml/minutes. Wavelength: UV 220 nm and 254 nm.

Method B: using the following elution gradient 10%-80% (solvent B) over 6 minutes and holding at 60% for 0.5 minutes at a flow rate of 0.8 ml/minutes. Wavelength: UV 220 nm and 254 nm.

Method C: using the following elution gradient 30%-90% (solvent B) over 6 minutes and holding at 60% for 0.5 minutes at a flow rate of 0.8 ml/minutes. Wavelength: UV 220 nm and 254 nm.

### SFC Analytical Separation

Instrument: Waters UPC2 analytical SFC (SFC-H). Column: ChiralCel OJ, 150×4.6 mm I.D., 3 μm. Mobile phase: A for CO2 and B for Ethanol (0.05% DEA). Gradient: B 40%. Flow rate: 2.5 mL/min. Back pressure: 100 bar. Column temperature: 35° C. Wavelength: 220 nm

### Preparative HPLC Purification

General Method: Preparative HPLC was performed on a Gilson UV/VIS-156 with UV detection at 220/254 nm Gilson 281 automatic collection.

Acidic condition: Two acid grading systems used: Hydrochloride acid and Formic acid.

Method A: Hydrochloride acid: YMC-Actus Triart C18 150×30 mm×5 um, Gradient used 0-100% acetonitrile with water and corresponding acid (0.05% HCl).

Method B: Formic acid: Phenomenex Synergi C18 150×30 mm×4 um, Gradient used 0-100% acetonitrile with water and corresponding acid (0.225% formic acid), the gradient shape was optimized for individual separations.

Neutral condition: Xtimate C18 150×25 mm×5 um, Gradient used 0-100% (water (10 mM NH₄HCO₃)-ACN), the gradient shape was optimized for individual separations.

Basic condition: Waters Xbridge Prep OBD C18 150×30 10 um, Gradient used 0-100% water (0.04% NH₃H₂O+10 mM NH₄HCO₃)-acetonitrile, the gradient shape was optimized for individual separations.

### Preparative HPLC-MS Purification

Columns Used:

Acid: Waters SunFire Prep, C18 Sum, OBD 19×100 mm

Base: Waters XSelect CSH Prep C18 5 um OBD 19×100 mm

Gradient Profile: 12 min Run: Initial conditions: A-95%: B-5%; hold at initial from 0.0-0.5 min; linear ramp from A-5% to variable B-% (typical range is from B-40% to B-75%) between 0.5-7.5 min; linear ramp from B-% to B-95% from 7.5-8.0 min; hold at A-5%:B-95% between 8.0-10.0 min; end of DAD/MS detection; linear ramp down to initial conditions between 10.0-10.5 min and hold at initial for 1.5 min.

Mobile Phase: Acid: A: 0.1% trifluoroacetic acid in water (v/v); Mobile phase B: 0.1% trifluoroacetic acid in acetonitrile (v/v). Base: A: 0.1% ammonia in water (v/v); Mobile phase B: 0.1% ammonia in acetonitrile (v/v)

### Preparative SFC Purification

Instrument: MG III preparative SFC (SFC-1). Column: ChiralCel OJ, 250×30 mm I.D., 5 μm. Mobile phase: A for CO2 and B for Ethanol (0.1% NH3H2O). Gradient: B 50%. Flow rate: 40 mL/min. Back pressure: 100 bar. Column temperature: 38° C. Wavelength: 220 nm. Cycle time: ˜8 min.

### ¹H-NMR

The NMR spectra were recorded on Bruker Avance III HD 500 MHz, Bruker Avance III 500 MHz, Bruker Avance III 400 MHz, Varian-400 VNMRS, or Varian-400 MR. Chemical shifts are expressed in parts per million (ppm) units. Coupling constants (J) are in units of hertz (Hz). Splitting patterns describe apparent multiplicities and are designated as s (single), d (double), t (triplet), dd (double doublet), dt (double triplet), dq (double quartet), m (multiplet), br (broad).

Typically, the compounds of Formula (I) can be prepared according to the schemes provided below. The following examples serve to illustrate the invention without limiting the scope thereof. Methods for preparing such compounds are described hereinafter Abbreviations:

Abbreviations used are those conventional in the art or the following:

| | |
|---|---|
| AcOH,: acetic acid | Min(s): minute(s) |
| EtOAc or EA: ethyl acetate | m/z: mass to charge ratio |
| LC and LCMS: liquid chromatography and | MeOH: methanol |
| liquid chromatography-mass spectrometry | |
| HRMS: high resolution mass spectrometry | Pd₂(dba)₃: |
| | Tris(dibenzylideneacetone)dipalladium(0) |
| ESI: electrospray ionization | MeCN: acetonitrile |
| DCM: dichloromethane | DMF: dimethylformamide |
| EtOH: ethanol | DMSO: dimethylsulfoxide |
| Na₂SO₄: sodium sulfate | Pd(OAc)₂: Palladium(II) acetate |
| DIPEA: diisopropyl ethylamine | SCX: strong cation exchange sorbent, solid |
| | phase purification reagent |
| T3P: 2,4,6-Tripropyl-1,3,5,2,4,6- | N2: nitrogen |
| trioxatriphosphorinane-2,4,6-trioxide solution | |
| BOP: (Benzotriazol-1- | Cs₂CO₃: cesium carbonate |
| yloxy)tris(dimethylamino)phosphonium | |
| hexafluorophosphate | |
| THF: tetrahydrofuran | LiOH: lithium hydroxide |
| HCl: hydrochloric acid | NBS: N-bromosuccinimide |
| NaHCO₃: sodium bicarbonate | ABPR: Automated Back Pressure Regulator |
| MBPR: manual back pressure regular | DEA” diethylamine |
| PE: petroleum ether | HATU: 1-[Bis(dimethylamino)methylene]- |
| | 1H-1,2,3-triazolo [4,5-b]pyridinium 3-oxid |
| | hexafluorophosphate |
| NIS: N-Iodosuccinimide | TFA: 2,2,2-trifluoroacetic acid |
| NaHMDS: Sodium bis(trimethylsilyl)amide | t-BuOK: Potassium t-butoxide |

For illustrative purposes, the reaction schemes depicted below provide potential routes for synthesizing the compounds of the present invention as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.
SCHEMES: Scheme I and II provide potential routes for making compounds of formula (I).

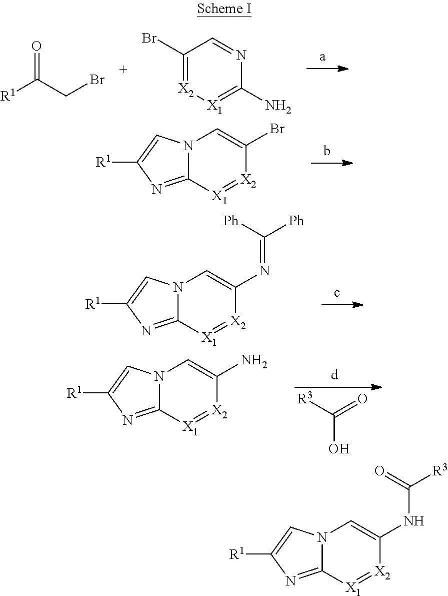

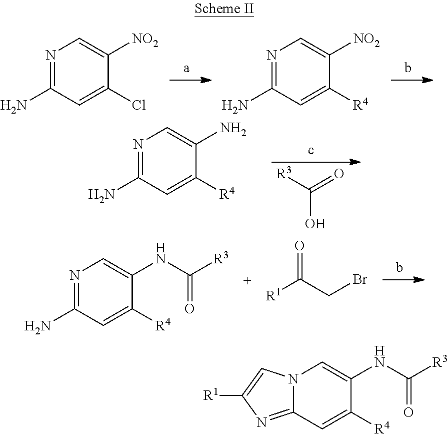

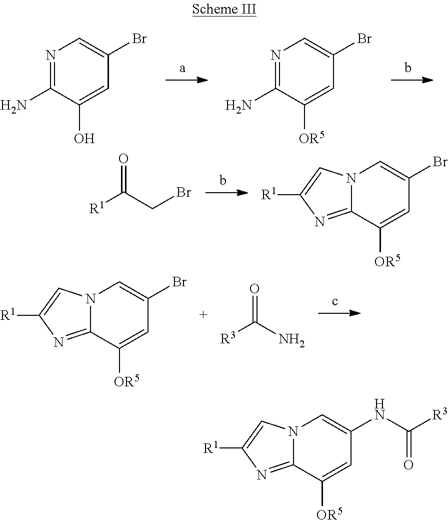

The starting materials for the above reaction scheme are commercially available or can be prepared according to methods known to one skilled in the art or by methods disclosed herein. In general, compounds of the invention can be prepared by the above reaction Schemes as follows:
Scheme I: a) Condensation of an appropriately substituted 2-aminopyridine with a 2-bromoketone to form the 5,6-heterocyclic core. b) Cross-coupling of benzophenone imine with the aryl bromide is then followed directly with hydrolysis of the imine (step c) furnishes the aniline which is then utilized in an amide coupling reaction to provide the target compound.
Scheme II: a) Displacement of aryl halide with nucleophile R⁴ followed by reduction of the nitro group (b) provides a diaminopyridine, which undergoes selective amide coupling (c) at the 5-amine group to furnish the requisite amide. d) Condensation of the resulting aminopyridine with an appropriately substituted bromoketone furnishes the target compound.
Scheme III: a) Mitsunobu reaction of the phenol with R⁵—OH, followed by condensation of the 2-aminopyridine moiety with the appropriately substituted bromoketone in step b provides the 8-alkoxyimidazopyridine. Step c: cross-coupling of the amide with the bromoimidazopyridine furnishes the target compound.

### Intermediate 1: 6-bromo-7-ethoxy-2-ethylimidazo[1,2-a]pyridine

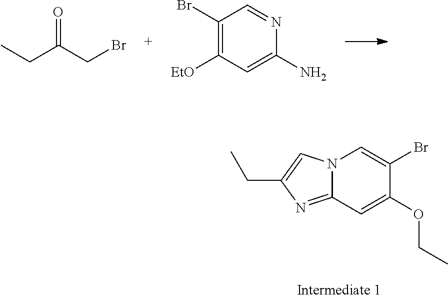

To a vial charged with 5-bromo-4-ethoxy-pyridin-2-amine (284 mg, 1.31 mmol), was added 1-bromobutan-2-one (218 mg, 1.4 mmol, 150 μL), EtOH (5 mL) followed by sodium bicarbonate (331 mg, 3.9 mmol) as a solid, open to air. The vial was sealed and heated at 85° C. for 18 h. LCMS indicated the presence of desired product 6-bromo-7-ethoxy-2-ethylimidazo[1,2-a]pyridine, as well as residual 5-bromo-4-ethoxy-pyridin-2-amine. Additional 1-bromobutan-2-one (218 mg, 1.4 mmol, 150 μL) was added and the mixture was heated an additional 4 h. Silica gel was added and the resulting mixture was purified by silica gel chromatography using a gradient of 5%-65% of a 3:1 EtOAc:EtOH blend in heptanes to provide Intermediate 1: 6-bromo-7-ethoxy-2-ethylimidazo[1,2-a]pyridine (158 mg, 45% yield). LCMS (ESI) m/z 269.0 (M+H)⁺. ¹H NMR (500 MHz, CHLOROFORM-d) δ ppm 1.31 (t, J=7.33 Hz, 3H) 1.36 (t, J=7.02 Hz, 3H) 2.73 (q, J=7.33 Hz, 2H) 3.94 (q, J=7.12 Hz, 2H) 6.69 (s, 1H) 7.02 (s, 1H) 7.34 (s, 1H)

### Intermediate 2: 6-bromo-2-cyclopropyl-7-ethoxyimidazo[1,2-a]pyridine

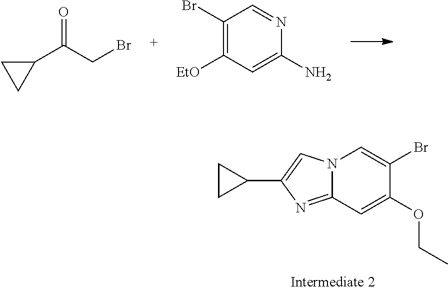 [SMILES: O=C(CBr)C1CC1 ; CCOc1cc(N)ncc1Br ; CCOc1cc2nc(C3CC3)cn2cc1Br]

A vial was charged with 5-bromo-4-ethoxy-pyridin-2-amine (270 mg, 1.24 mmol), 2-bromo-1-cyclopropyl-ethanone (235 mg, 1.44 mmol) followed by EtOH (5 mL). To this mixture was added sodium bicarbonate (330 mg, 3.93 mmol) as a solid, open to air. The vial was sealed and heated at 85° C. for 18 h at which time additional 2-bromo-1-cyclopropyl-ethanone (235 mg, 1.44 mmol) was added and the mixture was heated at 85° C. for 4 h. The mixture was transferred to a 30 mL vial and silica was added. The mixture was dried and loaded into a dry loading column. The residue was then eluted through a 40 g Reveleris 40 micron silica gel column (Grace X2 automated purification machine) using a gradient of 5%-65% of a 3:1EtOAc:EtOH blend in heptanes to provide Intermediate 2: 6-bromo-2-cyclopropyl-7-ethoxy-imidazo[1,2-a]pyridine. LCMS (ESI) m/z 280.9 (M+H)⁺. ¹H NMR (500 MHz, CHLOROFORM-d) δ ppm 0.92-0.99 (m, 4H) 1.52 (t, J=7.02 Hz, 3H) 1.93-2.03 (m, 1H) 4.11 (q, J=7.33 Hz, 3H) 6.83 (s, 1H) 7.18 (s, 1H) 8.14 (s, 1H).

### Intermediate 3a: 4-ethoxypyridine-2,5-diamine

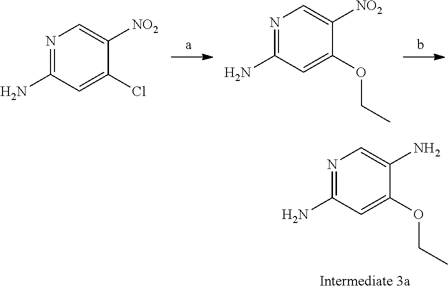

Step a: A solution of 4-chloro-5-nitropyridin-2-amine (1.0 g, 5.8 mmol) and sodium ethoxide (588 mg, 8.64 mmol) in EtOH (20 mL) was stirred in a sealed tube at 80° C. for 1 h. The described reaction was run in 5 concurrent batches which were combined and then H₂O (200 mL) was added to the suspension to form a yellow precipitate. The mixture was filtered to obtain a yellow solid, which was successively washed with H₂O (50 mL), EtOH (10 mL) and petroleum ether (50 mL) and then dried in vacuo to provide 4-ethoxy-5-nitropyridin-2-amine (4.1 g, 78% yield) as yellow solid. ¹H NMR: (400 MHz DMSO-d₆) δ 8.62 (s, 1H), 7.16 (br s, 2H), 6.06 (s, 1H), 4.13 (q, J=6.8 Hz, 2H), 1.37 (t, J=7.2 Hz, 3H).

Step b: To a solution of 4-ethoxy-5-nitropyridin-2-amine (4.1 g, 22.4 mmol) in MeOH (150 mL) was added palladium on carbon (2 g, 10 wt %). The suspension was stirred at 30° C. under H₂ (15 psi) for 2.5 h. The mixture was then filtered through a celite pad, washing with MeOH (100 mL). The filtrate was concentrated in vacuo to provide Intermediate 3a: 4-ethoxypyridine-2,5-diamine (3.4 g, 99% yield) as a black brown solid. ¹H NMR: (400 MHz DMSO-d₆) δ 7.29 (s, 1H), 6.00 (s, 1H), 4.94 (s, 2H), 3.97 (q, J=7.2 Hz, 2H), 3.83 (s, 2H), 1.36-1.31 (m, 3H).

### Intermediate 3b: 6-(difluoromethyl)picolinic Acid

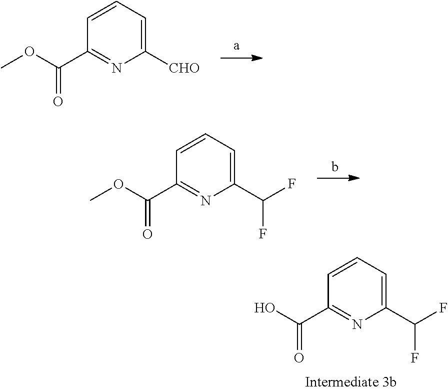

Step a: To a solution of methyl 6-formylpicolinate (4 g, 24 mmol) and EtOH (0.4 mL, 6.84 mmol, 0.4 mL, 2.82e-1 eq) in DCM (140 mL) was added N-ethyl-N-(trifluoro-sulfanyl)ethanamine (15.6 g, 96.9 mmol, 12.80 mL) at 0° C. The mixture was stirred at 0° C. for 1.5 h. Then the mixture was poured into ice-water (300 mL) and extracted with DCM (3×50 mL). The combined organic layers were washed with brine (2×50 mL), dried over Na₂SO₄, filtered and concentrated in vacuo to provide methyl 6-(difluoromethyl)picolinate (4.5 g, 92% yield) was obtained as brown oil, which was used without further purification. ¹H NMR: (400 MHz CDCl₃) δ 8.26-8.26 (m, 1H), 8.05-8.00 (m, 1H), 7.87-7.85 (m, 1H), 6.90-6.61 (m, 1H), 4.03 (s, 3H).

Step b: A mixture of methyl 6-(difluoromethyl)picolinate (4.47 g, 23.9 mmol) in HCl (12 M, 22 mL) was stirred at 90° C. for 6 h. The mixture was concentrated in vacuo and then diluted with water (50 mL). The resulting mixture was extracted with DCM (3×50 mL). The combined organic layers were washed with brine (2×20 mL), dried over Na₂SO₄, filtered and concentrated in vacuo to provide Intermediate 3b: 6-(difluoromethyl)picolinic acid (2.5 g, 60% yield) as an off-white solid, which was used in the next step without further purification. ¹H NMR (400 MHz, CDCl₃) δ 8.39-8.37 (m, 1H), 8.18-8.14 (m, 1H), 7.96-7.94 (m, 1H), 6.85-6.58 (m, 1H).

### Intermediate 3c: N-(6-amino-4-ethoxypyridin-3-yl)-6-(difluoromethyl)picolinamide

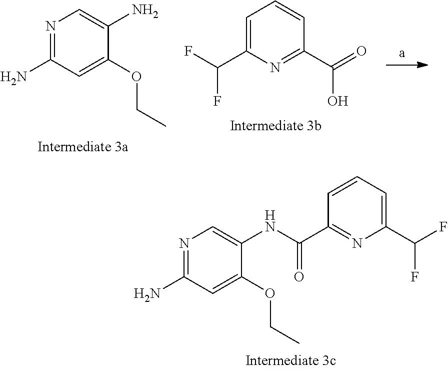 [SMILES: CCOc1cc(N)ncc1N ; O=C(O)c1cccc(C(F)F)n1 ; CCOc1cc(N)ncc1NC(=O)c1cccc(C(F)F)n1]

Step a: To a solution of Intermediate 3b: 6-(difluoromethyl)picolinic acid (2.7 g, 15.7 mmol) in DMF (24 mL) was added HATU (6.55 g, 17.2 mmol), DIPEA (8.2 mL, 47 mmol) and Intermediate 3a, 4-ethoxypyridine-2,5-diamine (2.9 g, 18.9 mmol). The mixture was stirred at 30° C. for 3 h. The mixture was diluted with H₂O (150 mL) and then extracted with EtOAc (4×150 mL). The combined organics were washed with H₂O (200 mL), brine (200 mL), dried over Na₂SO₄, then filtered and concentrated in vacuo. The residue was purified by silica gel chromatography using a gradient of 2% EtOAc in petroleum ether to 100% EtOAc to provide Intermediate 3c: N-(6-amino-4-ethoxypyridin-3-yl)-6-(difluoromethyl)picolinamide (3.5 g, 11.3 mmol, 72% yield) as a brown solid. LCMS (ESI) m/z 309.3 (M+H)⁺.

### Intermediate 4: N-(6-amino-4-ethoxypyridin-3-yl)-6-(trifluoromethyl)picolinamide

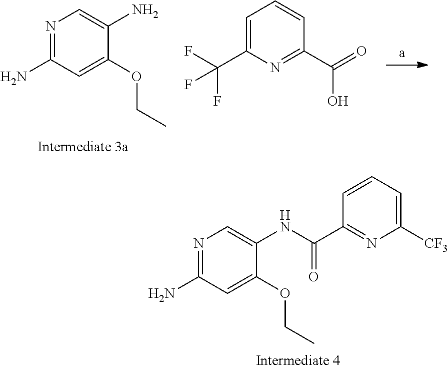 [SMILES: CCOc1cc(N)ncc1N ; O=C(O)c1cccc(C(F)(F)F)n1 ; CCOc1cc(N)ncc1NC(=O)c1cccc(C(F)(F)F)n1]

Intermediate 4 N-(6-amino-4-ethoxypyridin-3-yl)-6-(trifluoromethyl)picolinamide was prepared by standard amide coupling in the same manner as Intermediate 3c, substituting commercially available 6-(trifluoromethyl)picolinic acid in place of Intermediate 3b: 6-(difluoromethyl)picolinic acid. LCMS (ESI) m/z 327.4 (M+H)⁺.

### Intermediate 5: 7-ethoxy-2-ethylimidazo[1,2-a]pyridin-6-amine

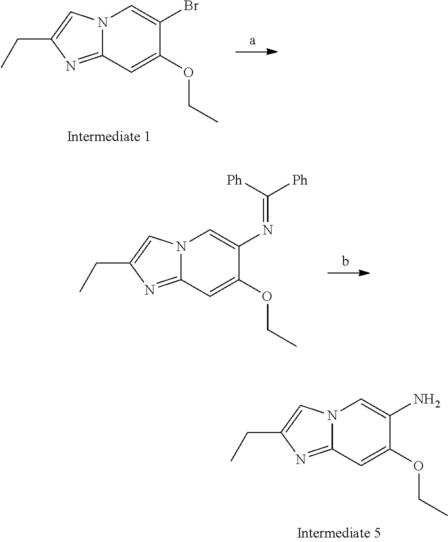 [SMILES: CCOc1cc2nc(CC)cn2cc1Br ; CCOc1cc2nc(CC)cn2cc1N=C(c1ccccc1)c1ccccc1 ; CCOc1cc2nc(CC)cn2cc1N]

Step a: In a vial, Intermediate 1: A vial was charged with 6-bromo-7-ethoxy-2-ethylimidazo[1,2-a]pyridine (1.6 g, 5.94 mmol), sodium tert-butoxide (799 mg, 8.32 mmol), Pd₂(dba)₃ (163 mg, 178 μmol), [1-(2-diphenylphosphanyl-1-naphthyl)-2-naphthyl]-diphenyl-phosphane (296 mg, 475 μmol). The vial was flushed with N₂, and then toluene (6 mL) was added, followed by diphenylmethanimine (1.29 g, 7.13 mmol, 1.20 mL). The resulting mixture was heated at 100° C. overnight. The reaction was then concentrated onto silica gel purified via column chromatography 5% 3:1 EtOAc:EtOH ramping to 100% 3:1 EtOAc:EtOH to give N-(7-ethoxy-2-ethyl-imidazo[1,2-a]pyridin-6-yl)-1,1-diphenyl-methanimine (1.00 g, 2.71 mmol, 46% yield) LCMS (ESI) m/z 370.5 (M+H)⁺.

Step b: N-(7-ethoxy-2-ethyl-imidazo[1,2-a]pyridin-6-yl)-1,1-diphenyl-methanimine (1.00 g, 2.71 mmol) in DCM (27 mL) and MeOH (27 mL) was treated with HCl (4 M in Dioxane, 1.7 mL) and stirred for 1 hour. The reaction was then concentrated, then purified via a SCX column, first eluting with MeOH, and then 2 M NH₃ in MeOH, to give Intermediate 5: 7-ethoxy-2-ethyl-imidazo[1,2-a]pyridin-6-amine (450.00 mg, 2.19 mmol, 80.81% yield) LCMS (ESI) m/z 206.3 (M+H)⁺.

### Intermediate 5b: 6-bromo-2-cyclopropyl-7-ethoxy-3-fluoroimidazo[1,2-a]pyridine

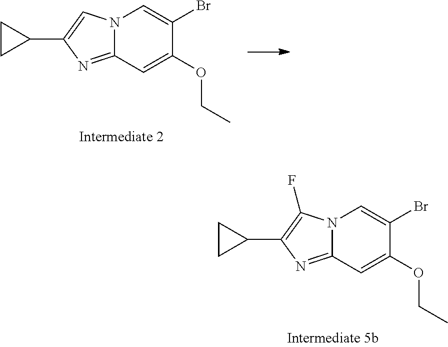 [SMILES: CCOc1cc2nc(C3CC3)cn2cc1Br ; CCOc1cc2nc(C3CC3)c(F)n2cc1Br]

N,N-dimethylpyridin-4-amine (DMAP) (35 mg, 284.5 μmol) and 1-(chloromethyl)-4-fluoro-1,4-diazoniabicyclo[2.2.2]octane; ditetrafluoroborate (111 mg, 313 μmol) were added to a mixture of Intermediate 2: 6-bromo-2-cyclopropyl-7-ethoxy-imidazo[1,2-a]pyridine (80 mg, 284 μmol) in CHCl₃ (2.9 mL) and water (0.7 mL) at 0° C. The mixture was stirred vigorously at 0° C. for 3 h, then allowed to warm to rt overnight. The reaction mixture was quenched with aq. sat. Na₂CO₃, extracted three times with EtOAc, dried over MgSO₄, filtered, concentrated, and purified by silica gel chromatography (0-70% of a 3:1 EtOAc:EtOH blend in heptanes) to obtain Intermediate 5b: 6-bromo-2-cyclopropyl-7-ethoxy-3-fluoroimidazo[1,2-a]pyridine (15.00 mg, 50.14 μmol, 17.62% yield) as an inseparable mixture with Intermediate 2: 6-bromo-2-cyclopropyl-7-ethoxyimidazo[1,2-a]pyridine. Mixture used without further purification in the next reaction. LCMS (ESI) m/z 300.9 (M+H)⁺.

### Intermediate 5c: 2-cyclopropyl-7-ethoxyimidazo[1,2-a]pyridin-6-amine

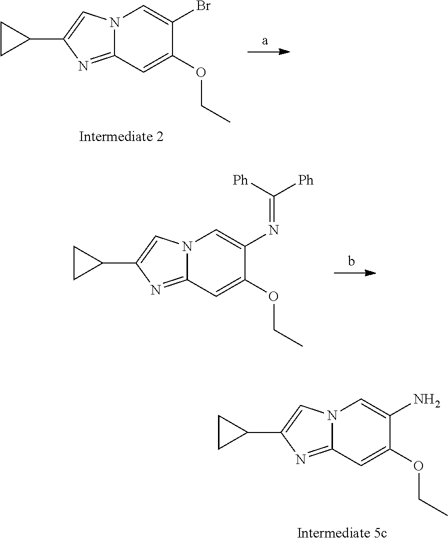 [SMILES: CCOc1cc2nc(C3CC3)cn2cc1Br ; CCOc1cc2nc(C3CC3)cn2cc1N=C(c1ccccc1)c1ccccc1 ; CCOc1cc2nc(C3CC3)cn2cc1N]

Step a: A vial was charged with Intermediate 2: 6-bromo-2-cyclopropyl-7-ethoxy-imidazo[1,2-a]pyridine (162 mg, 577 μmol), diphenylmethanimine (126 mg, 693 μmol), sodium tert-butoxide (78 mg, 808 μmol), Pd₂(dba)₃ (16 mg, 17 μmol), and [1-(2-diphenylphosphanyl-1-naphthyl)-2-naphthyl]-diphenyl-phosphane (29 mg, 47 μmol). The vial was sealed with a septum cap and flushed with N₂. Toluene (3 mL) was added, and the mixture was stirred at rt for 2 min while sparging with N₂. To this mixture was added diphenylmethanimine (126 mg, 693 μmol, 116 μL) and the resulting mixture was heated at 90° C. for 18 h. The mixture was concentrated onto silica gel and purified by silica gel chromatography using 5%-60% MeOH in EtOAc to provide N-(2-cyclopropyl-7-ethoxyimidazo[1,2-a]pyridin-6-yl)-1,1-diphenylmethanimine (185 mg, 84% yield). LCMS (ESI) m/z 382.2 (M+H)⁺.

Step b: A vial was charged with N-(2-cyclopropyl-7-ethoxy-imidazo[1,2-a]pyridin-6-yl)-1,1-diphenyl-methanimine (185 mg, 485 μmol) followed by DCM (1 mL), and MeOH (1 mL). To the resulting solution was added a dioxane solution of hydrogen chloride (4 M, 200 μL). The mixture was maintained at rt for 1 h at which time it was concentrated in vacuo to provide Intermediate 5c: 2-cyclopropyl-7-ethoxyimidazo[1,2-a]pyridin-6-amine hydrochloride (115 mg, 453 μmol, 93% yield) which was used directly without further purification. LCMS (ESI) m/z 218.1 (M+H)⁺. ¹H NMR (500 MHz, METHANOL-d₄) δ ppm 0.88-0.97 (m, 4H) 1.50 (t, J=7.02 Hz, 3H) 1.93-2.00 (m, 1H) 4.16 (q, J=6.71 Hz, 2H) 6.69 (s, 1H) 7.20 (s, 1H) 7.65 (s, 1H)

### Intermediate 6: 6-bromo-2-cyclopropyl-7-methoxyimidazo[1,2-a]pyridine

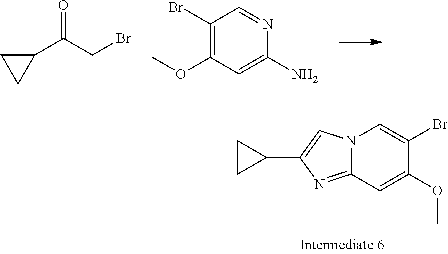

Intermediate 6: 6-bromo-2-cyclopropyl-7-methoxyimidazo[1,2-a]pyridine was prepared in a similar manner to that described for Intermediate 2, substituting 5-bromo-4-methoxypyridin-2-amine in place of 5-bromo-4-ethoxypyridin-2-amine. LCMS (ESI) m/z 267.0 (M+H)⁺.

### Intermediate 7: 2-cyclopropyl-6-nitroimidazo[1,2-a]pyridine

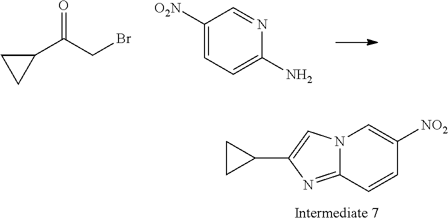 [SMILES: O=C(CBr)C1CC1 ; Nc1ccc([N+](=O)[O-])cn1 ; O=[N+]([O-])c1ccc2nc(C3CC3)cn2c1]

A mixture of 5-nitropyridin-2-amine (1 g, 7.2 mmol) and 2-bromo-1-cyclopropyl-ethanone (1.2 g, 7.2 mmol, 0.7 mL) in MeCN (9 mL) and toluene (2.4 mL) was stirred at 100° C. for 14 h. Additional 2-bromo-1-cyclopropyl-ethanone (1.2 g, 7.2 mmol, 0.7 mL) was added and the reaction mixture was stirred at 100° C. overnight before being cooled to rt. MeOH and silica were added, the mixture was concentrated and purified by silica gel chromatography using a gradient of 0-70% of a 3:1 EtOAc:EtOH blend in heptanes to obtain Intermediate 7: 2-cyclopropyl-6-nitroimidazo[1,2-a]pyridine (590 mg, 2.9 mmol, 40% yield). LCMS (ESI) m/z 204.1 (M+H)⁺, ¹H NMR (400 MHz, METHANOL-d₄) δ ppm 0.93-0.97 (m, 2H) 1.03-1.09 (m, 2H) 2.09 (tt, J=8.28, 4.89 Hz, 1H) 7.51 (dt, J=10.04, 0.75 Hz, 1H) 7.84 (s, 1H) 7.99 (dd, J=10.04, 2.26 Hz, 1H) 9.56 (dd, J=2.26, 0.75 Hz, 1H).

### Intermediate 8: 6-bromo-2-cyclopropylimidazo[1,2-a]pyrazine

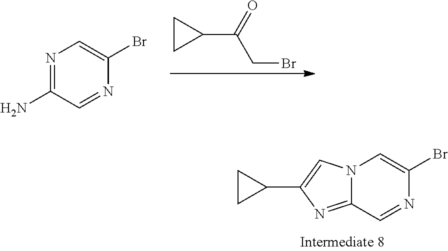

6-bromo-2-cyclopropylimidazo[1,2-a]pyrazine was prepared in a similar manner to that described for Intermediate 1, starting from 5-bromopyrazin-2-amine. LCMS (ESI) m/z 240.0 (M+H)+.

### Example 1: N-(7-ethoxy-2-ethylimidazo[1,2-a]pyridin-6-yl)-6-(trifluoromethyl)picolinamide

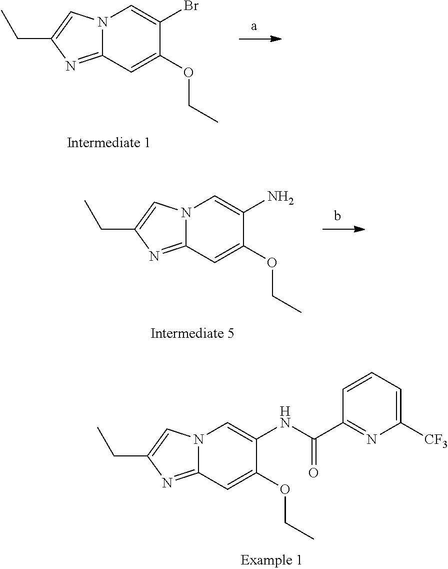 [SMILES: CCOc1cc2nc(CC)cn2cc1Br ; CCOc1cc2nc(CC)cn2cc1N ; CCOc1cc2nc(CC)cn2cc1NC(=O)c1cccc(C(F)(F)F)n1]

Step a: A vial was charged with Intermediate 1: 6-bromo-7-ethoxy-2-ethylimidazo[1,2-a]pyridine (153 mg, 569 μmol), copper (I) iodide (22 mg, 114 μmol), (2S)-pyrrolidine-2-carboxylic acid (27 mg, 228 μmol), and potassium carbonate (118 mg, 853 μmol). The vial was sealed with a septum cap and flushed with N₂. DMSO (1 mL) was added and the mixture stirred at rt for 2 min while sparging with N₂. To this mixture was added an aqueous solution of 28% ammonium hydroxide (0.1 mL, 0.8 mmol). The resulting mixture was heated at 90° C. for 18 h. The mixture was then concentrated onto silica gel and purified by silica gel chromatography using 5%-60% MeOH in EtOAc to provide 6-bromo-7-ethoxy-2-ethylimidazo[1,2-a]pyridine (57 mg, 49% yield). LCMS (ESI) m/z 206.1 (M+H)⁺.

Step b: To a flask charged with 6-(trifluoromethyl)pyridine-2-carboxylic acid (58 mg, 305 μmol), was added 7-ethoxy-2-ethyl-imidazo[1,2-a]pyridin-6-amine (57 mg, 278 μmol) followed by DCM (3 mL). To this mixture was added DIPEA (833 μmol, 150 μL) followed by an EtOAc solution of 2,4,6-Tripropyl-1,3,5,2,4,6-trioxatriphosphorinane-2,4,6-trioxide solution (T3P®) (555 μmol, 250 μL, 50 wt. %). The mixture was maintained at rt for 18 h and was then partitioned between 1 N aqueous NaOH (10 mL) and EtOAc (20 mL). The layers were separated, and the aqueous layer was extracted with EtOAc (3×15 mL). The combined organic layers were dried (Na₂SO₄) and concentrated. The crude residue was purified by silica gel chromatography, using a gradient of 5%-55% of a 3:1 EtOAc:EtOH blend in heptanes to provide Example 1: N-(7-ethoxy-2-ethylimidazo[1,2-a]pyridin-6-yl)-6-(trifluoromethyl)picolinamide (49 mg, 127 μmol, 46% yield). LCMS (ESI) m/z 379.1 (M+H)⁺. ¹H NMR (500 MHz, methanol-d4) δ 9.35 (d, J=2.44 Hz, 1H), 8.39 (d, J=7.94 Hz, 1H), 8.28 (t, J=7.94 Hz, 1H), 8.04 (d, J=7.94 Hz, 1H), 7.34-7.43 (m, 1H), 6.73-6.88 (m, 1H), 4.13-4.28 (m, 2H), 2.71 (q, J=7.33 Hz, 2H), 1.56 (t, J=7.02 Hz, 3H), 1.27-1.37 (m, 3H).

### Example 2: N-(2-cyclopropyl-7-ethoxyimidazo[1,2-a]pyridin-6-yl)-6-(trifluoromethyl)picolinamide

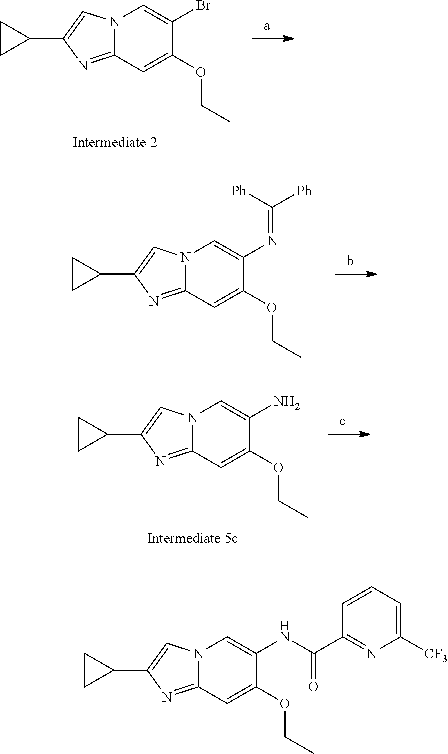 [SMILES: CCOc1cc2nc(C3CC3)cn2cc1Br ; CCOc1cc2nc(C3CC3)cn2cc1N=C(c1ccccc1)c1ccccc1 ; CCOc1cc2nc(C3CC3)cn2cc1N ; CCOc1cc2nc(C3CC3)cn2cc1NC(=O)c1cccc(C(F)(F)F)n1]

Step a: A vial was charged with Intermediate 2: 6-bromo-2-cyclopropyl-7-ethoxy-imidazo[1,2-a]pyridine (162 mg, 577 μmol), diphenylmethanimine (126 mg, 693 μmol), sodium tert-butoxide (78 mg, 808 μmol), Pd₂(dba)₃ (16 mg, 17 μmol), and [1-(2-diphenylphosphanyl-1-naphthyl)-2-naphthyl]-diphenyl-phosphane (29 mg, 47 μmol). The vial was sealed with a septum cap and flushed with N₂. Toluene (3 mL) was added, and the mixture was stirred at rt for 2 min while sparging with N₂. To this mixture was added diphenylmethanimine (126 mg, 693 μmol, 116 μL) and the resulting mixture was heated at 90° C. for 18 h. The mixture was concentrated onto silica gel and purified by silica gel chromatography using 5%-60% MeOH in EtOAc to provide N-(2-cyclopropyl-7-ethoxyimidazo[1,2-a]pyridin-6-yl)-1,1-diphenylmethanimine (185 mg, 84% yield). LCMS (ESI) m/z 382.2 (M+H)⁺.

Step b: A vial was charged with N-(2-cyclopropyl-7-ethoxy-imidazo[1,2-a]pyridin-6-yl)-1,1-diphenyl-methanimine (185 mg, 485 μmol) followed by DCM (1 mL), MeOH (1 mL). To the resulting solution was added a dioxane solution of hydrogen chloride (4 M, 200 μL). The mixture was maintained at rt for 1 h, at which time it was concentrated in vacuo to provide Intermediate 5c: 2-cyclopropyl-7-ethoxyimidazo[1,2-a]pyridin-6-amine hydrochloride (115 mg, 453 μmol, 93% yield), which was used directly without further purification. LCMS (ESI) m/z 218.1 (M+H)⁺.

Step c: A flask was charged with 6-(trifluoromethyl)pyridine-2-carboxylic acid (101 mg, 529 μmol), Intermediate 5c: 2-cyclopropyl-7-ethoxyimidazo[1,2-a]pyridin-6-amine hydrochloride (115 mg, 529 μmol) followed by EtOAc (5 mL). To the resulting heterogeneous mixture was added DIPEA (0.3 mL, 1.6 mmol) followed by an EtOAc solution of 2,4,6-Tripropyl-1,3,5,2,4,6-trioxatriphosphorinane-2,4,6-trioxide solution (T3P®) (0.5 mL, 1.1 mmol, 50 wt %).

The mixture was stirred at rt for 18 h, at which time it was partitioned between 1 N aqueous NaOH (10 mL) and EtOAc (20 mL). The layers were separated, and the aqueous layer was extracted with EtOAc (3×15 mL). The combined organic layers were dried (Na₂SO₄) and concentrated. The crude residue was taken up in minimal DMSO and filtered through a 0.45 micron frit, and purified by reverse-phase HPLC (Gilson, column: SunFire Prep C18 OBD, 5 micrometers, 30×50 nM column) eluting with a %1 TFA modified gradient of 25%-90% MeCN in H₂O to provide N-(2-cyclopropyl-7-ethoxyimidazo[1,2-a]pyridin-6-yl)-6-(trifluoromethyl)picolinamide (50 mg, 18% yield). LCMS (ESI) m/z 391.2 (M+H)⁺. ¹H NMR (500 MHz, DMSO-d6) ä ppm 0.83-0.94 (m, 2H) 1.04-1.18 (m, 2H) 1.53 (t, J=7.02 Hz, 3H) 2.09-2.19 (m, 1H) 4.41 (q, J=6.71 Hz, 2H) 7.31 (s, 1H) 8.08 (s, 1H) 8.29 (dd, J=7.33, 1.22 Hz, 1H) 8.40-8.53 (m, 2H) 9.62-9.72 (m, 1H) 10.53 (s, 1H).

### Example 3: N-(2-cyclopropyl-7-ethoxyimidazo[1,2-a]pyridin-6-yl)-6-(difluoromethyl)picolinamide

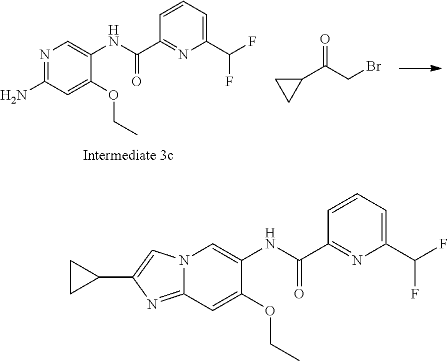 [SMILES: CCOc1cc(N)ncc1NC(=O)c1cccc(C(F)F)n1 ; O=C(CBr)C1CC1 ; CCOc1cc2nc(C3CC3)cn2cc1NC(=O)c1cccc(C(F)F)n1]

Example 3: N-(2-cyclopropyl-7-ethoxyimidazo[1,2-a]pyridin-6-yl)-6-(difluoromethyl)picolinamide was prepared from Intermediate 3c through the cyclization reaction with 2-bromo-1-cyclopropyl-ethanone in a similar manner to that described for the preparation of Intermediate 2. LCMS (ESI) m/z 373.2 (M+H)⁺. 1H NMR (400 MHz, METHANOL-d4) δ ppm 0.91-0.99 (m, 2H), 1.16-1.24 (m, 2H), 1.68 (t, J=6.90 Hz, 3H), 2.09-2.18 (m, 1H), 4.46 (q, J=6.86 Hz, 2H), 6.88 (t, J=56.0 Hz, 1H), 7.25 (s, 1H), 7.80 (s, 1H), 7.99 (d, J=7.78 Hz, 1H), 8.29 (t, J=7.66 Hz, 1H), 8.42 (d, J=7.78 Hz, 1H), 9.73 (s, 1H).

### Example 4: N-(2-cyclopropyl-7-methoxyimidazo[1,2-a]pyridin-6-yl)-6-(difluoromethyl)picolinamide

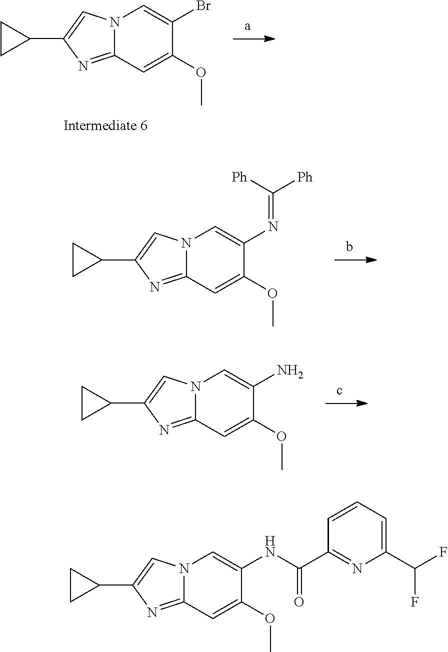

Example 4: N-(2-cyclopropyl-7-methoxyimidazo[1,2-a]pyridin-6-yl)-6-(difluoromethyl)picolinamide was prepared from Intermediate 6 through the same synthetic sequence as described for the preparation of Example 2, except for substituting Intermediate 3b: 6-(difluoromethyl)picolinic acid in place of 6-(trifluoromethyl)picolinic acid in step c. LCMS (ESI) m/z 359.1 (M+H)⁺. 1H NMR (400 MHz, METHANOL-d4) δ ppm 9.60 (s, 1H), 8.28 (dd, J=0.88, 7.91 Hz, 1H), 8.16 (t, J=7.78 Hz, 1H), 7.87 (d, J=7.78 Hz, 1H), 7.67 (s, 1H), 7.19 (s, 1H), 6.79 (t, J=56.0 Hz, 1H), 4.14 (s, 3H), 1.95-2.07 (m, 1H), 1.04-1.13 (m, 2H), 0.80-0.90 (m, 2H).

### Example 5: N-(7-(cyclobutylmethoxy)-2-cyclopropylimidazo[1,2-a]pyridin-6-yl)-6-(difluoromethyl)picolinamide

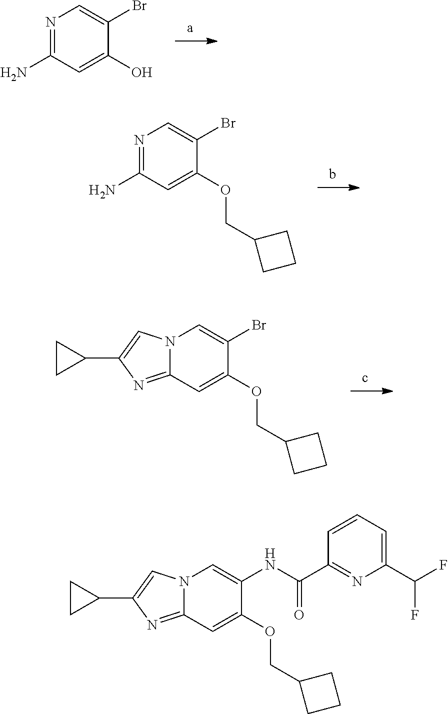 [SMILES: Nc1cc(O)c(Br)cn1 ; Nc1cc(OCC2CCC2)c(Br)cn1 ; Brc1cn2cc(C3CC3)nc2cc1OCC1CCC1 ; O=C(Nc1cn2cc(C3CC3)nc2cc1OCC1CCC1)c1cccc(C(F)F)n1]

Step a: To a vial charged with a mixture of cyclobutylmethanol (86 mg, 1.0 mmol), triphenylphosphine (262 mg, 1.0 mmol), 2-amino-5-bromo-pyridin-4-ol (189 mg, 1.00 mmol) in THF (3 mL) was added diisopropyl azodicarboxylate (196 μL, 1.00 mmol) dropwise at rt. After addition, the vial was capped, and the reaction mixture was stirred at rt for 16 h. The mixture was then concentrated to give a crude residue, which was purified by silica gel chromatography using a gradient of 0-100% of a 3:1 blend of EtOAc:EtOH in heptanes to give 5-bromo-4-(cyclobutylmethoxy)pyridin-2-amine (386.7 mg, 1.50 mmol, 150.39% yield; mixed with triphenylphosphine oxide, used directly in the next step). LCMS (ESI) m/z 257.0 (M+H)⁺.

Step b: To a vial charged with 5-bromo-4-(cyclobutylmethoxy)pyridin-2-amine (100 mg, 389 μmol), 2-bromo-1-cyclopropyl-ethanone (60 mg, 370 μmol) was added EtOH (3 mL), followed by sodium bicarbonate (98 mg, 1.2 mmol) as a solid in one portion, open to air. The vial was sealed and heated at 85° C. for 18 h at which time the reaction suspension was filtered through a Celite filter and washed with MeOH. The filtrate was dried (Na₂SO₄) and the residue was loaded into a dry loading column. The residue was then eluted through a 40 g Reveleris 40 micron silica gel column using a gradient of 0%-100% of a blend of 3:1 EtOAc:EtOH in heptane. Clean fractions were collected and concentrated to provide 6-bromo-7-(cyclobutylmethoxy)-2-cyclopropyl-imidazo[1,2-a]pyridine (20 mg, 62 μmol, 16% yield). LCMS (ESI) m/z 321.1 (M+H)⁺.

Step c: A vial was charged with 6-bromo-7-(cyclobutylmethoxy)-2-cyclopropyl-imidazo[1,2-a]pyridine (20 mg, 62 μmol), Pd(OAc)₂ (2.8 mg, 12 μmol), (5-diphenylphosphanyl-9,9-dimethyl-xanthen-4-yl)-diphenyl-phosphane (14.4 mg, 24.9 μmol), and Cs₂CO₃ (40.6 mg, 124 μmol) was evacuated, backfilled with N₂ and closed with a screw cap with septa. A dioxane (1 mL) solution of 6-(difluoromethyl)pyridine-2-carboxamide (21 mg, 124 μmol) was added via a syringe at 22° C. The vial was sealed and heated at 100° C. for 16 h (turned yellow-brown). LCMS showed formation of the desired product as a major peak. Then the reaction mixture was allowed to cool to room temperature, filtered through Celite, concentrated and purified by silica gel chromatography using a gradient of 0-100% of a 3:1 blend of EtOAc:EtOH in heptanes to afford N-(7-(cyclobutylmethoxy)-2-cyclopropylimidazo[1,2-a]pyridin-6-yl)-6-(difluoromethyl)picolinamide (13.8 mg, 33.5 μmol, 53.7% yield) as a white solid. LCMS (ESI) m/z 413.2 (M+H)⁺. ¹H NMR (400 MHz, METHANOL-d4) Q ppm 0.88-0.99 (m, 2H), 1.15-1.24 (m, 2H), 2.00-2.19 (m, 5H), 2.24-2.40 (m, 2H), 2.98-3.09 (m, 1H), 4.35 (d, J=6.53 Hz, 2H), 6.83 (t, J=56.0 Hz, 1H), 7.28 (s, 1H), 7.77 (s, 1H), 7.97 (d, J=7.78 Hz, 1H), 8.27 (t, J=7.91 Hz, 1H), 8.40 (d, J=7.78 Hz, 1H), 9.73 (s, 1H).

### Example 6: N-(2-cyclopropyl-7-(2-(2-oxopyridin-1(2H)-yl)propoxy)imidazo[1,2-a]pyridin-6-yl)-6-(difluoromethyl)picolinamide

N-(2-cyclopropyl-7-(2-(2-oxopyridin-1(2H)-yl)propoxy)imidazo[1,2-a]pyridin-6-yl)-6-(difluoromethyl)picolinamide was prepared through the same synthetic sequence as described for the preparation of Example 5: except for substituting 1-(2-hydroxy-1-methyl-ethyl)pyridin-2-one in place of cyclobutylmethanol in Step a. LCMS (ESI) m/z 480.2 (M+H)⁺. ¹H NMR (400 MHz, CHLOROFORM-d) δ ppm 0.86-1.02 (m, 4H), 1.73 (d, J=7.28 Hz, 3H), 1.91-2.03 (m, 1H), 4.25-4.41 (m, 2H), 5.62-5.77 (m, 1H), 6.09 (td, J=6.78, 1.26 Hz, 1H), 6.56 (d, J=9.04 Hz, 1H), 6.69-7.04 (m, 2H), 7.20-7.31 (m, 2H), 7.60 (dd, J=6.90, 1.88 Hz, 1H), 7.87 (d, J=7.53 Hz, 1H), 8.13 (t, J=7.78 Hz, 1H), 8.42 (d, J=7.78 Hz, 1H), 9.42 (s, 1H).

Enantiomers of Example 6 were separated by chiral SFC under the conditions of:
CHIRALPAK IA 30×250 mm, 5 μm
Method: 40% EtOH w/0.1% DEA in CO₂ (flow rate: 100 mL/min, ABPR 120 bar, MBPR 40 psi, column temp 40 deg C.)

### Example 7: (R)—N-(2-cyclopropyl-7-(2-(2-oxopyridin-1(2H)-yl)propoxy)imidazo[1,2-a]pyridin-6-yl)-6-(difluoromethyl)picolinamide

Example 7 is peak 1 from the chiral SFC separation and the stereochemistry is arbitrarily assigned. (>99% ee)

### Example 8: (S)—N-(2-cyclopropyl-7-(2-(2-oxopyridin-1(2H)-yl)propoxy)imidazo[1,2-a]pyridin-6-yl)-6-(difluoromethyl)picolinamide

Example 8 is peak 2 from the chiral SFC separation and the stereochemistry is arbitrarily assigned. (91% ee)

### Example 9: N-(7-(benzyloxy)-2-cyclopropylimidazo[1,2-a]pyridin-6-yl)-6-(difluoromethyl)picolinamide

N-(7-(benzyloxy)-2-cyclopropylimidazo[1,2-a]pyridin-6-yl)-6-(difluoromethyl)picolinamide was prepared through the same synthetic sequence as described for the preparation of Example 5, except for substituting phenylmethanol in place of cyclobutylmethanol in Step a. LCMS (ESI) m/z 435.2 (M+H)⁺. ¹H NMR (400 MHz, CHLOROFORM-d) δ ppm 0.88-0.99 (m, 4H), 1.94-2.02 (m, 1H), 5.16 (s, 2H), 6.28 (t, J=56.0 Hz, 1H), 6.97 (s, 1H), 7.22 (s, 1H), 7.40-7.52 (m, 3H), 7.52-7.58 (m, 2H), 7.79 (d, J=7.78 Hz, 1H), 8.05 (t, J=7.91 Hz, 1H), 8.33 (dd, J=7.78, 0.75 Hz, 1H), 9.39 (s, 1H), 10.49 (s, 1H).

### Example 10: N-(2-cyclopropyl-7-hydroxyimidazo[1,2-a]pyridin-6-yl)-6-(difluoromethyl)picolinamide

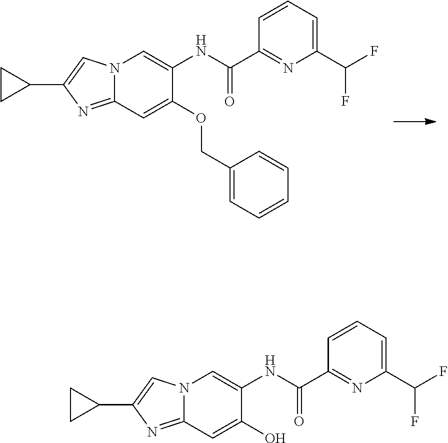 [SMILES: O=C(Nc1cn2cc(C3CC3)nc2cc1OCc1ccccc1)c1cccc(C(F)F)n1 ; O=C(Nc1cn2cc(C3CC3)nc2cc1O)c1cccc(C(F)F)n1]

N-(7-(benzyloxy)-2-cyclopropylimidazo[1,2-a]pyridin-6-yl)-6-(difluoromethyl)picolinamide (28 mg, 64.5 μmol), and Palladium on Carbon (13.7 mg, 12.9 μmol, 10% purity) were suspended in methanol (2 mL) and ethyl acetate (2 mL) in a pressure vessel. The vessel was pressurized with 15 psi of H₂. The reaction was stirred for 2 h at 22° C. and then filtered through Celite and the volatiles were removed under vacuum to afford N-(2-cyclopropyl-7-hydroxyimidazo[1,2-a]pyridin-6-yl)-6-(difluoromethyl)picolinamide (22.1 mg, 64.2 μmol, 99% yield). LCMS (ESI) m/z 345.1 (M+H)⁺. ¹H NMR (400 MHz, METHANOL-d4) δ ppm 0.83-0.84 (m, 2H), 1.01-1.11 (m, 2H), 1.91-2.04 (m, 1H), 6.41 (br s, 1H), 6.88 (t, J=56.0 Hz, 1H), 7.33 (br s, 1H), 7.94 (d, J=7.78 Hz, 1H), 8.23 (t, J=7.78 Hz, 1H), 8.37 (d, J=8.03 Hz, 1H), 9.33 (br s, 1H).

### Example 11: N-(2-cyclopropyl-7-(((2S,3S,4S)-3-ethyl-4-fluoro-5-oxopyrrolidin-2-yl)methoxy)imidazo[1,2-a]pyridin-6-yl)-6-(difluoromethyl)picolinamide

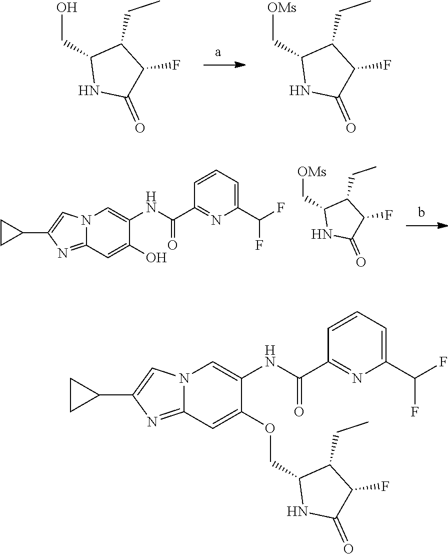

Step a: To a solution of (3S,4S,5S)-4-ethyl-3-fluoro-5-(hydroxymethyl)pyrrolidin-2-one (50 mg, 310 μmol) in dichloromethane (2 mL) was added triethylamine (94 mg, 931 μmol, 130 μL) and followed by methanesulfonyl chloride (53 mg, 465 μmol, 36.0 μL) at 0° C. The resulting mixture was stirred at 25° C. for 1 h. It was then concentrated in vacuo and the residue was re-suspended in DMF and used without further manipulation in the next step assuming quantitative yield. Step b: A 4 mL vial equipped with a stir bar was charged with N-(2-cyclopropyl-7-hydroxy-imidazo[1,2-a]pyridin-6-yl)-6-(difluoromethyl)pyridine-2-carboxamide (15 mg, 43 μmol), a DMF solution of [(2S,3S,4S)-3-ethyl-4-fluoro-5-oxo-pyrrolidin-2-yl]methyl methanesulfonate (10.4 mg, 44 μmol), and cesium carbonate (28 mg, 87 μmol) and DMF (300 μL). The heterogeneous solution was heated to 110° C. and stirred over night (16 hours). The reaction was then allowed to cool to room temperature, filtered and directly purified by mass-directed HPLC on a reverse phase column. The fractions were dried to afford N-(2-cyclopropyl-7-(((2S,3S,4S)-3-ethyl-4-fluoro-5-oxopyrrolidin-2-yl)methoxy)imidazo[1,2-a]pyridin-6-yl)-6-(difluoromethyl)picolinamide (16.4 mg, 27.3 μmol, 62.6% yield) as a white solid. LCMS (ESI) m/z 488.2 (M+H)⁺. ¹H NMR (400 MHz, METHANOL-d4) δ ppm 0.90-1.03 (m, 2H), 1.06-1.16 (m, 3H), 1.17-1.28 (m, 3H), 1.71-1.86 (m, 2H), 2.00-2.19 (m, 1H), 4.25-4.43 (m, 2H), 4.53 (dd, J=9.91, 4.39 Hz, 1H), 4.96-5.07 (m, 1H), 7.00 (t, J=56.0 Hz, 1H), 7.37 (s, 1H), 7.82 (s, 1H), 7.96-8.03 (m, 1H), 8.24-8.32 (m, 1H), 8.39-8.46 (m, 1H), 9.76 (s, 1H).

### Example 12: 6-(difluoromethyl)-N-(7-ethoxy-2-(3-methoxypropyl)imidazo[1,2-a]pyridin-6-yl)picolinamide

A sealed tube was charged with a mixture of Intermediate 3c: N-(6-amino-4-ethoxypyridin-3-yl)-6-(difluoromethyl)picolinamide (300 mg, 0.97 mmol), 1-chloro-5-methoxypentan-2-one (440 mg, 2.9 mmol), NaHCO₃ (245 mg, 2.9 mmol) and EtOH (6 mL). The tube was sealed and the resulting mixture was heated at 80° C. for 16 h at which time the mixture was cooled to room temperature and diluted with H₂O (25 mL). A yellow precipitate was formed and collected by vacuum filtration. The filter cake was purified by Prep-HPLC (column: Shim-pack C18 150×25×10 μm; eluting with a 0.2% formic acid modified gradient of 25%-46% MeCN in H₂O to provide 6-(difluoromethyl)-N-(7-ethoxy-2-(3-methoxypropyl)imidazo[1,2-a]pyridin-6-yl)picolinamide as a white solid. LCMS (ESI) m/z 405.1 (M+H)⁺. ¹H NMR (400 MHz DMSO-d₆) δ 10.46 (s, 1H), 9.39 (s, 1H), 8.38-8.26 (m, 2H), 8.05-7.95 (m, 1H), 7.64 (s, 1H), 7.30-6.93 (m, 2H), 4.22 (q, J=6.8 Hz, 2H), 3.37 (br t, J=6.8 Hz, 2H), 3.24 (s, 3H), 2.69-2.60 (m, 2H), 1.95-1.83 (m, 2H), 1.49 (t, J=7.2 Hz, 3H).

### Example 13: 6-(difluoromethyl)-N-(7-ethoxy-2-(2-methoxyethyl)imidazo[1,2-a]pyridin-6-yl)picolinamide

6-(Difluoromethyl)-N-(7-ethoxy-2-(2-methoxyethyl)imidazo[1,2-a]pyridin-6-yl)picolinamide was prepared in a similar fashion to that described for Example 12: using 1-chloro-4-methoxybutan-2-one in place of 1-chloro-5-methoxypentan-2-one to provide 6-(difluoromethyl)-N-(7-ethoxy-2-(2-methoxyethyl)imidazo[1,2-a]pyridin-6-yl)picolinamide as a yellow solid. LCMS (ESI) m/z 391.0 (M+H)⁺. ¹H NMR (400 MHz DMSO-d₆) δ 10.47 (s, 1H), 9.40 (s, 1H), 8.37-8.28 (m, 2H), 8.06-7.97 (m, 1H), 7.67 (s, 1H), 7.30-6.93 (m, 2H), 4.23 (q, J=6.8 Hz, 2H), 3.64 (t, J=7.2 Hz, 2H), 3.26 (s, 3H), 2.85 (t, J=6.8 Hz, 2H), 1.49 (t, J=7.2 Hz, 3H).

### Example 14: 6-(difluoromethyl)-N-(7-ethoxy-2-((tetrahydrofuran-3-yl)methyl)imidazo[1,2-a]pyridin-6-yl)picolinamide

A sealed tube was charged with Intermediate 3c: N-(6-amino-4-ethoxypyridin-3-yl)-6-(difluoromethyl)picolinamide (300 mg, 1.0 mmol), 1-chloro-3-(tetrahydrofuran-3-yl)propan-2-one (475 mg, 2.9 mmol), NaHCO₃ (245 mg, 2.9 mmol) and EtOH (6 mL). The tube was sealed and the mixture was heated at 80° C. for 16 h. The mixture was cooled to room temperature and concentrated in vacuo. The residue was partitioned between EtOAc (30 mL) and H₂O (30 mL). The layers were separated, and the aqueous layer was extracted with EtOAc (2×30 mL). The combined organic layers were dried over Na₂SO₄, filtered and concentrated in vacuo. The residue was purified by Prep-HPLC (column: Phenomenex Synergi C18 150×30 mm×4 um; eluting with a 0.2% formic acid modified gradient of 25%-55% MeCN in H₂O to provide 6-(difluoromethyl)-N-(7-ethoxy-2-((tetrahydrofuran-3-yl)methyl)imidazo[1,2-a]pyridin-6-yl)picolinamide (126 mg, 31% yield) as a yellow solid. LCMS (ESI) m/z 417.2 (M+H)⁺. ¹H NMR (400 MHz DMSO-d₆) δ 10.47 (s, 1H), 9.39 (s, 1H), 8.36-8.26 (m, 2H), 8.05-7.96 (m, 1H), 7.68 (s, 1H), 7.30-6.94 (m, 2H), 4.23 (q, J=6.8 Hz, 2H), 3.81-3.71 (m, 2H), 3.64 (q, J=7.6 Hz, 1H), 3.41 (br dd, J=6.4, 8.4 Hz, 2H), 2.71-2.64 (m, 2H), 2.08-1.93 (m, 1H), 1.68-1.55 (m, 1H), 1.49 (t, J=7.2 Hz, 3H).

### Example 15: (S)-6-(difluoromethyl)-N-(7-ethoxy-2-((tetrahydrofuran-3-yl)methyl)imidazo[1,2-a]pyridin-6-yl)picolinamide

The compound of Example 15 was obtained as Peak 1 from chiral SFC separation of Example 14: the stereochemistry was arbitrarily assigned. Column: Chiralpak IG 30×250 mm, 5 μm; Elution gradient: 30% EtOH w/0.1% DEA in CO₂ (flow rate: 100 mL/min, ABPR 120 bar, MBPR 40 psi, column temp 40 deg C.) to provide (S)-6-(difluoromethyl)-N-(7-ethoxy-2-((tetrahydrofuran-3-yl)methyl)imidazo[1,2-a]pyridin-6-yl)picolinamide (>99% ee). LCMS (ESI) m/z 417.2 (M+H)⁺, ¹H NMR (500 MHz, CDCl₃) δ ppm 1.62 (t, J=6.90 Hz, 3H) 1.65-1.79 (m, 1H) 2.01-2.16 (m, 1H) 2.70-2.80 (m, 1H) 2.80-2.86 (m, 2H) 3.55 (dd, J=8.28, 6.02 Hz, 1H) 3.80 (q, J=7.78 Hz, 1H) 3.88-3.99 (m, 2H) 4.22 (q, J=6.86 Hz, 2H) 6.50-6.85 (m, 1H) 6.88 (s, 1H) 7.25 (s, 1H) 7.85 (d, J=7.78 Hz, 1H) 8.12 (t, J=7.78 Hz, 1H) 8.40 (d, J=7.53 Hz, 1H) 9.43 (s, 1H) 10.54 (s, 1H).

### Example 16: (R)-6-(difluoromethyl)-N-(7-ethoxy-2-((tetrahydrofuran-3-yl)methyl)imidazo[1,2-a]pyridin-6-yl)picolinamide

The compound of Example 16 was obtained as Peak 2 from chiral SFC separation of Example 14: the stereochemistry was arbitrarily assigned. Column: Chiralpak IG 30×250 mm, 5 μm; Elution gradient: 30% EtOH w/0.1% DEA in CO₂ (flow rate: 100 mL/min, ABPR 120 bar, MBPR 40 psi, column temp 40° C.) to provide (R)-6-(difluoromethyl)-N-(7-ethoxy-2-((tetrahydrofuran-3-yl)methyl)imidazo[1,2-a]pyridin-6-yl)picolinamide (>91% ee). LCMS (ESI) m/z 417.2 (M+H)+, ¹H NMR (500 MHz, CDCl₃) δ ppm 1.62 (t, J=6.90 Hz, 3H) 1.65-1.79 (m, 1H) 2.01-2.16 (m, 1H) 2.70-2.80 (m, 1H) 2.80-2.86 (m, 2H) 3.55 (dd, J=8.28, 6.02 Hz, 1H) 3.80 (q, J=7.78 Hz, 1H) 3.88-3.99 (m, 2H) 4.22 (q, J=6.86 Hz, 2H) 6.50-6.85 (m, 1H) 6.88 (s, 1H) 7.25 (s, 1H) 7.85 (d, J=7.78 Hz, 1H) 8.12 (t, J=7.78 Hz, 1H) 8.40 (d, J=7.53 Hz, 1H) 9.43 (s, 1H) 10.54 (s, 1H).

### Example 17: 6-(difluoromethyl)-N-(7-ethoxy-2-(1-hydroxy-2-methylpropan-2-yl)imidazo[1,2-a]pyridin-6-yl)picolinamide

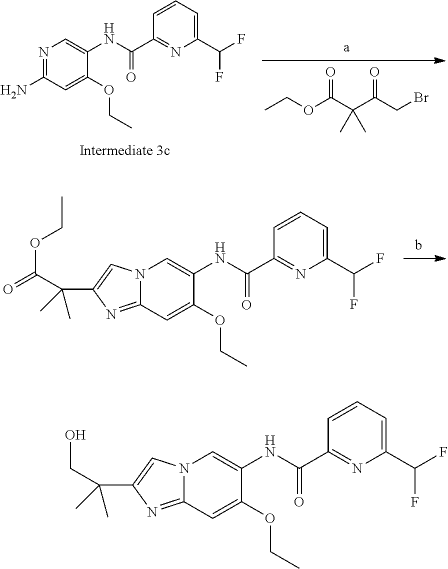 [SMILES: CCOc1cc(N)ncc1NC(=O)c1cccc(C(F)F)n1 ; CCOC(=O)C(C)(C)C(=O)CBr ; CCOC(=O)C(C)(C)c1cn2cc(NC(=O)c3cccc(C(F)F)n3)c(OCC)cc2n1 ; CCOc1cc2nc(C(C)(C)CO)cn2cc1NC(=O)c1cccc(C(F)F)n1]

Step a: Intermediate 3c was condensed with ethyl 4-bromo-2,2-dimethyl-3-oxobutanoate following the procedure described for Intermediate 1, substituting ethyl 4-bromo-2,2-dimethyl-3-oxobutanoate in place of 1-bromobutan-2-one to provide ethyl 2-(6-(6-(difluoromethyl)picolinamido)-7-ethoxyimidazo[1,2-a]pyridin-2-yl)-2-methylpropanoate. LCMS (ESI) m/z 447.1 (M+H)⁺, ¹H NMR (400 MHz, METHANOL-d₄) δ ppm 1.14 (t, J=7.03 Hz, 3H) 1.35-1.42 (m, 3H) 1.53 (s, 6H) 3.94-4.03 (m, 2H) 4.06 (q, J=7.03 Hz, 2H) 6.46-6.77 (m, 2H) 7.35-7.42 (m, 1H) 7.68 (br d, J=7.28 Hz, 1H) 7.93-8.08 (m, 2H) 9.10-9.16 (m, 1H).

Step b: To a solution of ethyl 2-[6-[[6-(difluoromethyl)pyridine-2-carbonyl]amino]-7-ethoxy-imidazo[1,2-a]pyridin-2-yl]-2-methyl-propanoate (120 mg, 269 μmol) in THF (1.8 ml) was added a THF solution of lithium aluminum hydride (1 M, 540 μL) at 0° C. After stirring for 2.5 h, the reaction mixture was quenched carefully with saturated aqueous NaCl solution (10 mL), diluted with EtOAc (10 mL) and saturated aqueous Rochelle salt solution (10 mL), and stirred vigorously for 20 min at rt. The aqueous phase was extracted with EtOAc, (3×10 mL), dried over MgSO₄, filtered, concentrated, and purified by silica column chromatography (0-80% gradient of a 3:1 EtOAc:EtOH blend in heptanes) to obtain 6-(difluoromethyl)-N-(7-ethoxy-2-(1-hydroxy-2-methylpropan-2-yl)imidazo[1,2-a]pyridin-6-yl)picolinamide (65 mg, 160.7 μmol, 60% yield). LCMS (ESI) m/z 405.1 (M+H)+, ¹H NMR (400 MHz, CHLOROFORM-d) δ ppm 1.29 (d, J=1.00 Hz, 6H) 1.52 (td, J=6.90, 0.75 Hz, 3H) 3.37-3.43 (m, 1H) 3.37-3.43 (m, 1H) 3.59 (d, J=1.00 Hz, 2H) 4.06-4.16 (m, 2H) 6.42-6.73 (m, 1H) 6.76 (s, 1H) 7.15 (s, 1H) 7.74 (d, J=7.78 Hz, 1H) 8.00 (t, J=7.78 Hz, 1H) 8.26 (d, J=7.78 Hz, 1H) 9.32 (s, 1H).

### Example 18: N-(7-ethoxy-2-(hydroxymethyl)imidazo[1,2-a]pyridin-6-yl)-6-(trifluoromethyl)picolinamide

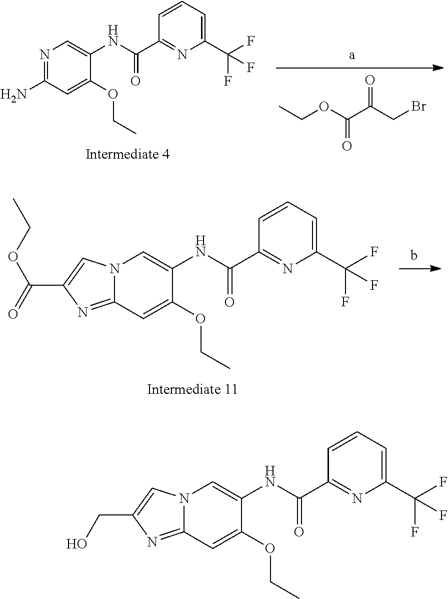

Step a: Intermediate 4 was reacted with ethyl 3-bromo-2-oxopropanoate following the procedure described for Intermediate 1, substituting ethyl 3-bromo-2-oxopropanoate in place of 1-bromobutan-2-one to provide Intermediate 11: ethyl 7-ethoxy-6-(6-(trifluoromethyl)picolinamido)imidazo[1,2-a]pyridine-2-carboxylate. LCMS (ESI) m/z 423.2 (M+H)+

Step b: N-(7-ethoxy-2-(hydroxymethyl)imidazo[1,2-a]pyridin-6-yl)-6-(trifluoromethyl)picolinamide was prepared in a similar manner to that described for Example 17: Step b, starting with ethyl 7-ethoxy-6-(6-(trifluoromethyl)picolinamido)imidazo[1,2-a]pyridine-2-carboxylate. LCMS (ESI) m/z 381.1 (M+H)⁺, ¹H NMR (400 MHz, METHANOL-d₄) δ ppm 1.66 (t, J=6.90 Hz, 2H) 1.63-1.70 (m, 1H) 4.45 (q, J=7.03 Hz, 2H) 4.79 (d, J=1.00 Hz, 2H) 7.30 (s, 1H) 7.97 (d, J=0.75 Hz, 1H) 8.12 (dd, J=7.78, 1.00 Hz, 1H) 8.36 (t, J=7.91 Hz, 1H) 8.48-10.00 (m, 1H) 8.49-8.54 (m, 1H) 8.49-8.54 (m, 1H) 8.51 (d, J=7.53 Hz, 1H).

### Example 19: N-(7-ethoxy-2-(2-hydroxypropan-2-yl)imidazo[1,2-a]pyridin-6-yl)-6-(trifluoromethyl)picolinamide

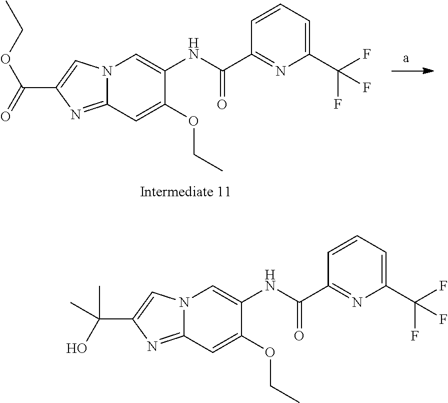 [SMILES: CCOC(=O)c1cn2cc(NC(=O)c3cccc(C(F)(F)F)n3)c(OCC)cc2n1 ; CCOc1cc2nc(C(C)(C)O)cn2cc1NC(=O)c1cccc(C(F)(F)F)n1]

Step a: To a solution of Intermediate 11: ethyl 7-ethoxy-6-(6-(trifluoromethyl)picolinamido)imidazo[1,2-a]pyridine-2-carboxylate (50 mg, 118.4 μmol) in THF (1 mL) at −78° C. was added methyl lithium (1.6 M Et₂O solution, 0.3 mL). After stirring for 3 h, the reaction mixture was quenched with saturated aqueous NHCl₄ solution. The aqueous phase was extracted with EtOAc (3×10 mL), dried over MgSO₄, filtered, concentrated, and purified by TFA-modified mass-directed HPLC on a reverse phase column (5-95% gradient of water to acetonitrile) to obtain N-(7-ethoxy-2-(2-hydroxypropan-2-yl)imidazo[1,2-a]pyridin-6-yl)-6-(trifluoromethyl)picolinamide (3.6 mg, 8% yield). LCMS (ESI) m/z 409.2 (M+H)⁺, ¹H NMR (400 MHz, METHANOL-d₄) δ ppm 1.61 (br d, J=5.02 Hz, 1H) 1.62-1.69 (m, 1H) 1.63-1.70 (m, 7H) 4.42-4.49 (m, 2H) 7.21-7.26 (m, 1H) 7.94 (s, 1H) 8.10-8.15 (m, 1H) 8.34-8.39 (m, 1H) 8.49-8.54 (m, 1H) 9.76-9.82 (m, 1H).

### Example 20: N-(7-ethoxy-2-(fluoromethyl)imidazo[1,2-a]pyridin-6-yl)-6-(trifluoromethyl)picolinamide

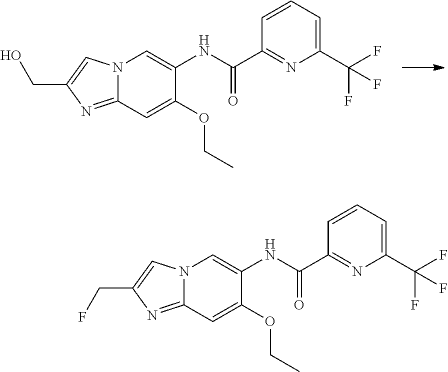 [SMILES: CCOc1cc2nc(CO)cn2cc1NC(=O)c1cccc(C(F)(F)F)n1 ; CCOc1cc2nc(CF)cn2cc1NC(=O)c1cccc(C(F)(F)F)n1]

To a solution of Example 18: N-(7-ethoxy-2-(hydroxymethyl)imidazo[1,2-a]pyridin-6-yl)-6-(trifluoromethyl)picolinamide (65 mg, 170.9 μmol) in DCM (2 mL) at 0° C. was added N-ethyl-N-(trifluoro-λ⁴-sulfanyl)ethanamine (188 μmol, 25 μL). After stirring for 1 h, the reaction mixture was concentrated and purified by TFA-modified mass-directed HPLC on a reverse phase column (5-95% gradient of water to acetonitrile) to obtain N-(7-ethoxy-2-(fluoromethyl)imidazo[1,2-a]pyridin-6-yl)-6-(trifluoromethyl)picolinamide (4.10 mg, 6% yield). LCMS (ESI) m/z 383.3 (M+H)+, ¹H NMR (400 MHz, METHANOL-d₄) δ ppm 1.66 (t, J=7.03 Hz, 3H) 4.47 (q, J=7.03 Hz, 2H) 5.55 (s, 1H) 5.67 (d, J=0.75 Hz, 1H) 7.35 (s, 1H) 8.13 (dd, J=7.91, 0.88 Hz, 1H) 8.22 (d, J=3.77 Hz, 1H) 8.32-8.39 (m, 1H) 8.52 (d, J=8.28 Hz, 1H) 9.84 (s, 1H).

### Example 21: N-(2-((1R,5S,6s)-3-oxabicyclo[3.1.0]hexan-6-yl)-7-ethoxyimidazo[1,2-a]pyridin-6-yl)-6-(difluoromethyl)picolinamide

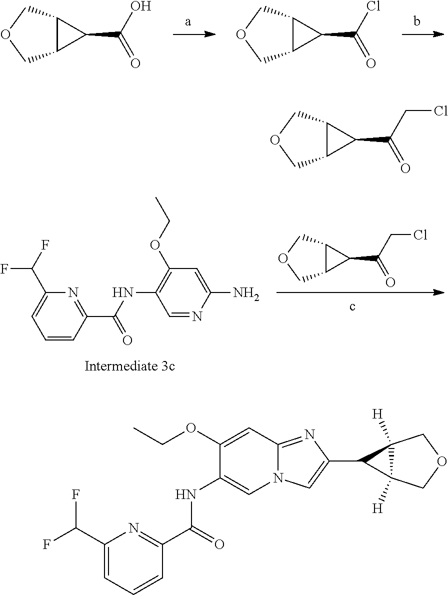

Step a: (1R,5S,6r)-3-oxabicyclo[3.1.0]hexane-6-carboxylic acid (500 mg, 3.90 mmol), a drop of DMF and oxalyl chloride (544 mg, 4.29 mmol), were mixed in DCM (20 mL) at rt. for 20 h. The reaction mixture was then concentrated under reduced pressure to provide (1R,5S,6r)-3-oxabicyclo[3.1.0]hexane-6-carbonyl chloride, which was used directly without further purification.

Step b: To a solution of (1R,5S,6r)-3-oxabicyclo[3.1.0]hexane-6-carbonyl chloride in MeCN (20 mL) was added diazomethyl(trimethyl)silane (2M in Et₂O, 2.2 mL). The resulting solution was maintained at rt. for 20 h, at which time aqueous hydrochloric acid (15%, 2 eq) was added and the resulting mixture was stirred for 4 h. To this mixture was added a saturated aqueous solution of NaHCO₃ in a portion-wise manner until pH≈7. The organic phase was separated, concentrated under reduced pressure and distilled at 20 mBar (b.p. 120° C.) to yield 1-((1R,5S,6r)-3-oxabicyclo[3.1.0]hexan-6-yl)-2-chloroethan-1-one (440 mg, 63% yield, 90% purity). 1H NMR (500 MHz, Chloroform-d) δ 4.20 (s, 2H), 3.95 (dd, J=8.2, 4.4 Hz, 2H), 3.78 (m, 2H), 2.28 (m, 2H), 2.18 (m, 1H).

Step c: 1-((1R,5S,6r)-3-oxabicyclo[3.1.0]hexan-6-yl)-2-chloroethan-1-one (278 mg, 1.56 mmol) and Intermediate 3c (462 mg, 1.20 mmol) were heated under reflux in propionitrile (20 mL) for 20 h. The mixture was cooled to rt and saturated aqueous Na₂CO₃ (5 mL) was added and the resulting mixture was stirred for additional 3 h. The organic phase was separated and concentrated under reduced pressure and the residue was purified by reverse-phase to yield N-(2-((1R,5S,6s)-3-oxabicyclo[3.1.0]hexan-6-yl)-7-ethoxyimidazo[1,2-a]pyridin-6-yl)-6-(difluoromethyl)picolinamide (37 mg, 7.4% yield). LCMS (ESI) m/z 415.2 (M+H)⁺; 1H NMR (400 MHz, DMSO-d6) δ 10.45 (s, 1H), 9.36 (s, 1H), 8.36-8.24 (m, 2H), 7.99 (m 1H), 7.67 (s, 1H), 7.10 (t, J=55.6 Hz, 1H), 7.01 (s, 1H), 4.20 (q, J=6.9 Hz, 2H), 3.88 (d, J=8.4 Hz, 2H), 3.69 (d, J=8.4 Hz, 2H), 2.01 (d, J=3.6 Hz, 2H), 1.77 (t, J=3.6 Hz, 1H), 1.48 (t, J=6.9 Hz, 3H).

### Example 22: N-(7-ethoxy-2-((tetrahydrofuran-3-yl)methyl)imidazo[1,2-a]pyridin-6-yl)-2-(1-methyl-1H-pyrazol-4-yl)oxazole-4-carboxamide

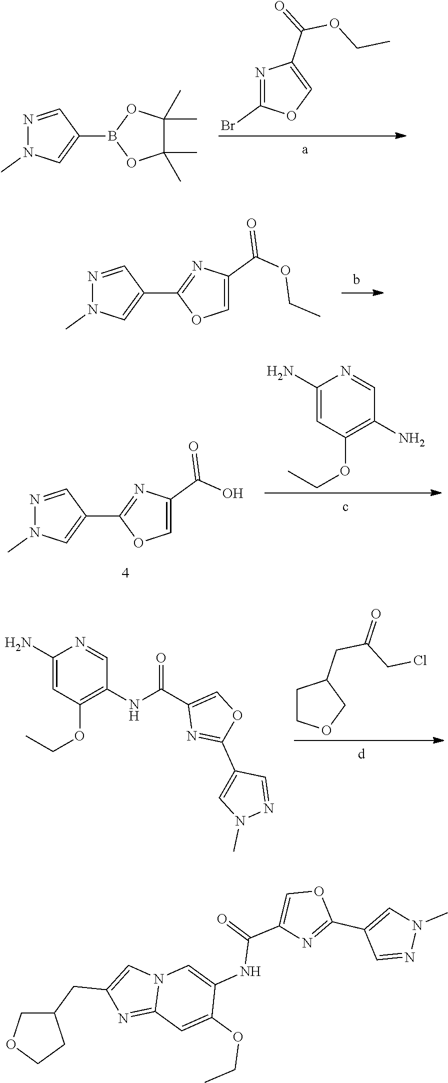

Step a: A 100-mL, three-necked, round-bottomed flask equipped with a magnetic stirring bar, condenser, and an argon gas inlet was charged with ethyl 2-bromooxazole-4-carboxylate (1.01 g, 4.58 mmol), 1-methyl-4-(4,4,5,5-tetramethyl-1,3,2-dioxaborolan-2-yl)-1H-pyrazole (1.00 g, 4.81 mmol), butyl alcohol (50 mL), H₂O (10 mL) and potassium phosphate tribasic (1.17 g, 5.50 mmol). The resulting mixture was stirred at rt for 15-20 min under argon atmosphere. Then Pd(dppf)Cl₂ (335 mg, 458 μmol) was added to the mixture and it was heated to reflux with stirring under an argon atmosphere overnight. After that, the solvents were evaporated under reduced pressure, the mixture was diluted with DCM and the resulting mixture was filtered through a short pad of Al₂O₃. The mother liquor was collected and concentrated in vacuo to give 1.5 g of crude product as a dark viscous oil, which was then purified by silica gel column chromatography to provide ethyl 2-(1-methyl-1H-pyrazol-4-yl)oxazole-4-carboxylate (400 mg, 1.81 mmol, 39% yield) as a pale yellow solid. LCMS (ESI) m/z 222 (M+H)⁺; 1H NMR (500 MHz, Chloroform-d): δ 8.17 (s, 1H), 8.03 (s, 1H), 7.99 (s, 1H), 4.42 (q, J=7.1 Hz, 2H), 3.98 (s, 3H), 1.41 (t, J=7.1 Hz, 3H).

Step b: A 25-mL round-bottomed flask, equipped with a magnetic stirrer, was charged with ethyl 2-(1-methyl-1H-pyrazol-4-yl)oxazole-4-carboxylate (300 mg, 1.35 mmol), K₂CO₃ (468 mg, 3.38 mmol, 2.5), H₂O (5 mL) and EtOH (3 mL). The resulting mixture was stirred at rt. for 48 h. Then, the EtOH was evaporated in vacuo, H₂O and activated carbon were added, and the mixture was filtered. The resulting filtrate was acidified with conc. HCl to pH 4-5 and a precipitate was formed, which was collected by vacuum filtration, washing with water to provide 2-(1-methyl-1H-pyrazol-4-yl)oxazole-4-carboxylic acid (210 mg, 1.09 mmol) as a white solid. LCMS (ESI) m/z 194 (M+H)⁺; 1H NMR (400 MHz, DMSO-d6): δ 8.66 (s, 1H), 8.38 (s, 1H), 7.94 (s, 1H), 3.91 (s, 3H).

Step c: To a solution of 2-(1-methyl-1H-pyrazol-4-yl)oxazole-4-carboxylic acid (200 mg, 1.04 mmol) in DMF (3 mL), was added HATU (433 mg, 1.138 mmol), DIPEA (401.4 mg, 3.1 mmol, 540 μL) and Intermediate 3a, 4-ethoxypyridine-2,5-diamine (190 mg, 1.24 mmol). The mixture was stirred at 35° C. overnight. After this period, the mixture was diluted with H₂O (10 mL) and extracted with EtOAc (3×10 mL). The combined organic layers were dried over Na₂SO₄, filtered and evaporated under reduced pressure to give 340 mg of crude product as dark-violet viscous oil, which contained 62% of the desired product N-(6-amino-4-ethoxypyridin-3-yl)-2-(1-methyl-1H-pyrazol-4-yl)oxazole-4-carboxamide by LCMS. This crude product was used in the next step without further purification. LCMS (ESI) m/z 329 (M+H)⁺.

Step d: A 25-mL round-bottomed flask, equipped with a magnetic stirrer and a condenser, was charged with N-(6-amino-4-ethoxypyridin-3-yl)-2-(1-methyl-1H-pyrazol-4-yl)oxazole-4-carboxamide (340 mg, 1.04 mmol), 1-chloro-3-(tetrahydrofuran-3-yl)propan-2-one (253 mg, 1.55 mmol, 1.5 eq), NaHCO₃ (130 mg, 1.55 mmol) and EtOH (10 mL). The resulting mixture was heated at 80° C. overnight. Then, the solvent was evaporated in vacuo, H₂O was added, and the product was extracted with CHCl₃ (3×10 mL). The combined organic layers were dried over Na₂SO₄ and concentrated. The residue was purified by HPLC to provide N-(7-ethoxy-2-((tetrahydrofuran-3-yl)methyl)imidazo[1,2-a]pyridin-6-yl)-2-(1-methyl-1H-pyrazol-4-yl)oxazole-4-carboxamide (125 mg, 0.29 mmol, 27% yield). LCMS (ESI) m/z 437 (M+H)⁺; 1H NMR (400 MHz, DMSO-d₆): δ 9.35 (s, 1H), 9.22 (s, 1H), 8.81 (s, 1H), 8.42 (s, 1H), 7.98 (s, 1H), 7.65 (s, 1H), 7.02 (s, 1H), 4.24 (q, J=6.9 Hz, 2H), 3.95 (s, 3H), 3.79-3.70 (m, 2H), 3.67-3.59 (m, 1H), 3.43-3.37 (m, 1H), 2.66 (d, J=7.0 Hz, 2H), 2.60-2.50 (m, 1H), 2.02-1.94 (m, 1H), 1.64-1.56 (m, 1H), 1.48 (t, J=6.9 Hz, 3H).

### Example 23: 6-(difluoromethyl)-N-(7-ethoxy-2-(3-hydroxypropyl)imidazo[1,2-a]pyridin-6-yl)picolinamide

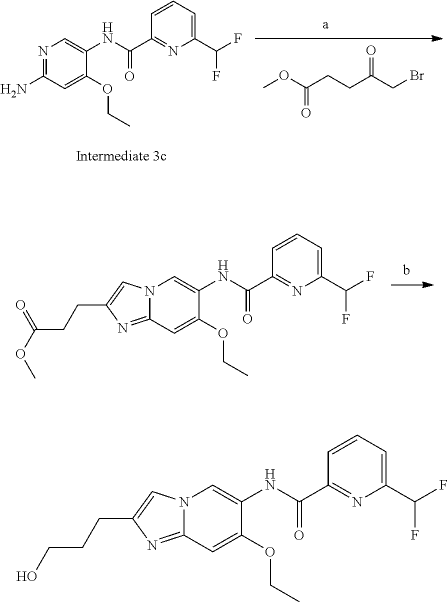

Step a: Methyl 3-(6-(6-(difluoromethyl)picolinamido)-7-ethoxyimidazo[1,2-a]pyridin-2-yl)propanoate was prepared in a similar manner to that described for Intermediate 1, substituting methyl 5-bromo-4-oxopentanoate in place of 1-bromobutan-2-one. LCMS (ESI) m/z 419.3 (M+H)+, ¹H NMR (400 MHz, METHANOL-d₄) δ ppm 1.44 (t, J=6.90 Hz, 3H) 2.61-2.68 (m, 2H) 2.84-2.89 (m, 2H) 3.56-3.61 (m, 3H) 4.05-4.13 (m, 2H) 6.53-6.81 (m, 2H) 7.29 (s, 1H) 7.76 (br d, J=7.53 Hz, 1H) 8.04-8.09 (m, 1H) 8.16 (br d, J=6.78 Hz, 1H) 9.20 (d, J=4.52 Hz, 1H).

Step b: 6-(Difluoromethyl)-N-(7-ethoxy-2-(3-hydroxypropyl)imidazo[1,2-a]pyridin-6-yl)picolinamide was prepared in a similar manner to that described for Example 17: starting with methyl 3-(6-(6-(difluoromethyl)picolinamido)-7-ethoxyimidazo[1,2-a]pyridin-2-yl)propanoate. LCMS (ESI) m/z 391.3 (M+H)⁺, ¹H NMR (400 MHz, METHANOL-d₄) δ ppm 1.57 (t, J=6.90 Hz, 3H) 1.91-2.00 (m, 2H) 2.76 (t, J=7.65 Hz, 2H) 3.65 (t, J=6.53 Hz, 2H) 4.21 (q, J=6.86 Hz, 2H) 6.64-6.93 (m, 2H) 7.41 (s, 1H) 7.88 (d, J=7.78 Hz, 1H) 8.16-8.22 (m, 1H) 8.26-8.30 (m, 1H) 9.34 (s, 1H).

### Example 24: N-(2-cyclopropyl-7-(4-(2,2-difluoroethyl)piperazin-1-yl)imidazo[1,2-a]pyridin-6-yl)-6-(trifluoromethyl)picolinamide

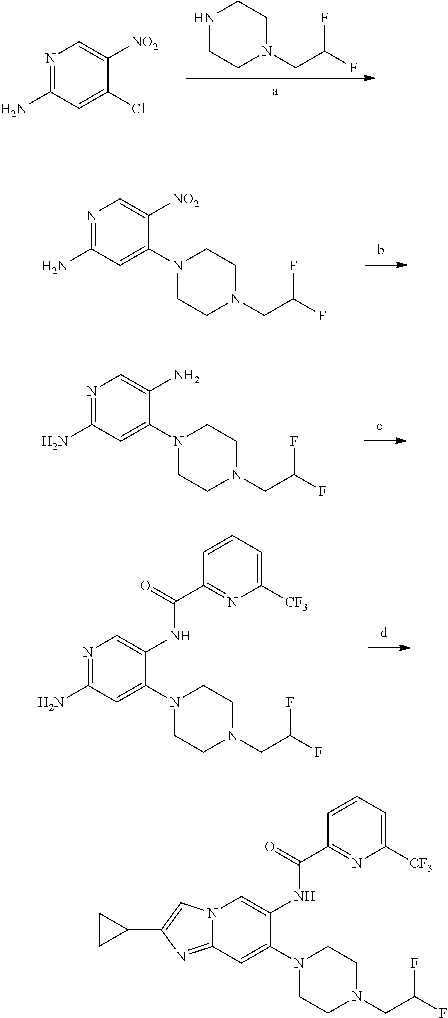 [SMILES: Nc1cc(Cl)c([N+](=O)[O-])cn1 ; FC(F)CN1CCNCC1 ; Nc1cc(N2CCN(CC(F)F)CC2)c([N+](=O)[O-])cn1 ; Nc1cc(N2CCN(CC(F)F)CC2)c(N)cn1 ; Nc1cc(N2CCN(CC(F)F)CC2)c(NC(=O)c2cccc(C(F)(F)F)n2)cn1 ; O=C(Nc1cn2cc(C3CC3)nc2cc1N1CCN(CC(F)F)CC1)c1cccc(C(F)(F)F)n1]

Step a: A mixture of 4-chloro-5-nitro-pyridin-2-amine (500 mg, 2.88 mmol), 1-(2,2-difluoroethyl)piperazine (455 mg, 2.9 mmol) and cesium carbonate (2.0 g, 6.0 mmol) in DMF (6 mL) was stirred 12 h at 90° C. The mixture was quenched with brine, extracted with EtOAc (3×50 mL), washed with brine (2×10 mL), dried over MgSO₄, filtered and concentrated to provide 4-(4-(2,2-difluoroethyl)piperazin-1-yl)-5-nitropyridin-2-amine which was used without further purification in the next reaction assuming 100% yield. LCMS (ESI) m/z 288.0 (M+H)⁺.

Step b: To a solution of crude 4-(4-(2,2-difluoroethyl)piperazin-1-yl)-5-nitropyridin-2-amine (827 mg, 2.88 mmol) in a 3:1 blend of EtOAc/EtOH (7.2 mL) was added palladium on carbon (150 mg, 10 wt %). The suspension was stirred at room temperature under 1 atmosphere of hydrogen (balloon) for 18 h. The mixture was then filtered through a celite pad, washing with MeOH (50 mL). The filtrate was concentrated in vacuo to provide 4-(4-(2,2-difluoroethyl)piperazin-1-yl)pyridine-2,5-diamine as a black solid which was used without further purification in the next reaction assuming 100% yield; LCMS (ESI) m/z 258.0 (M+H)⁺.

Step c: N-(6-amino-4-(4-(2,2-difluoroethyl)piperazin-1-yl)pyridin-3-yl)-6-(trifluoromethyl)picolinamide was prepared in a similar manner to that described for Intermediate 3c starting from 4-(4-(2,2-difluoroethyl)piperazin-1-yl)-5-nitropyridin-2-amine instead of 4-ethoxy-5-nitropyridin-2-amine. LCMS (ESI) m/z 431.0 (M+H)⁺, ¹H NMR (400 MHz, METHANOL-d₄) δ ppm 1.37-1.40 (m, 1H) 2.82-2.89 (m, 5H) 3.00-3.07 (m, 4H) 5.85-6.18 (m, 1H) 6.41 (s, 1H) 8.08 (dd, J=7.91, 0.88 Hz, 1H) 8.32 (t, J=7.91 Hz, 1H) 8.48 (d, J=7.78 Hz, 1H) 8.83 (s, 1H).

Step d: N-(2-cyclopropyl-7-(4-(2,2-difluoroethyl)piperazin-1-yl)imidazo[1,2-a]pyridin-6-yl)-6-(trifluoromethyl)picolinamide was prepared from N-(6-amino-4-(4-(2,2-difluoroethyl)piperazin-1-yl)pyridin-3-yl)-6-(trifluoromethyl)picolinamide through the cyclization reaction with 2-bromo-1-cyclopropyl-ethanone in a similar manner to that described for the preparation of Intermediate 2. LCMS (ESI) m/z 495.1 (M+H)⁺, ¹H NMR (500 MHz, METHANOL-d₄) δ ppm 0.95-1.01 (m, 2H) 1.20-1.27 (m, 2H) 2.16 (tt, J=8.85, 4.27 Hz, 1H) 3.37-3.50 (m, 1H) 3.38-3.47 (m, 9H) 3.47-3.63 (m, 1H) 6.18-6.44 (m, 1H) 7.51 (d, J=1.83 Hz, 1H) 7.85-7.91 (m, 1H) 8.18 (dd, J=7.94, 1.22 Hz, 1H) 8.40 (t, J=7.63 Hz, 1H) 8.58 (d, J=7.94 Hz, 1H) 9.74-9.80 (m, 1H).

### Example 25: N-(2-cyclopropyl-7-(cyclopropylmethoxy)imidazo[1,2-a]pyridin-6-yl)-6-(difluoromethyl)picolinamide

N-(2-cyclopropyl-7-(cyclopropylmethoxy)imidazo[1,2-a]pyridin-6-yl)-6-difluoromethyl)picolinamide was prepared in a similar manner to that described for Example 5: substituting cyclopropylmethanol in place of cyclobutylmethanol. LCMS (ESI) m/z 399.3 (M+H)+, ¹H NMR (400 MHz, METHANOL-d₄) δ ppm 0.54-0.62 (m, 2H) 0.84 (q, J=6.11 Hz, 2H) 0.90-0.97 (m, 2H) 1.15-1.21 (m, 2H) 1.52 (br s, 1H) 2.07-2.16 (m, 1H) 4.27 (br d, J=7.28 Hz, 2H) 6.71-7.04 (m, 1H) 7.22 (s, 1H) 7.79 (s, 1H) 7.99 (br d, J=7.28 Hz, 1H) 8.29 (t, J=7.91 Hz, 1H) 8.40 (br s, 1H) 9.73 (br s, 1H).

### Intermediate 17: 5-bromo-4-(2,2,2-trifluoroethoxy)pyridin-2-amine

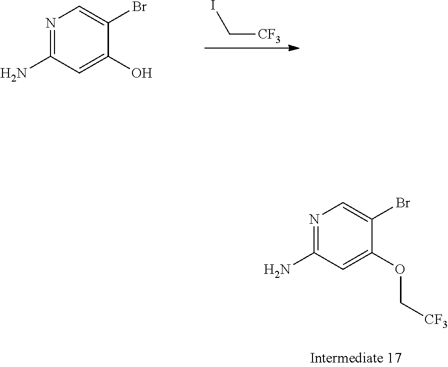 [SMILES: Nc1cc(O)c(Br)cn1 ; FC(F)(F)CI ; Nc1cc(OCC(F)(F)F)c(Br)cn1]

A mixture of 2-amino-5-bromo-pyridin-4-ol (500 mg, 2.65 mmol), K₂CO₃ (1.1 g, 7.95 mmol), and 1,1,1-trifluoro-2-iodo-ethane (834 mg, 3.98 mmol, 390 μL) in DMF (4.4 mL) was stirred at 110° C. for 14 h. After cooling to rt, the reaction mixture was diluted with brine (100 mL) and EtOAc (100 mL), the layers were separated and the aqueous layer was extracted with EtOAc (3×50 mL). The combined organic layers were washed with brine (2×10 mL), dried over MgSO₄, filtered, concentrated, and purified via silica column chromatography using a gradient of 0-70% of a 3:1 EtOAc:EtOH blend in heptanes to obtain 5-bromo-4-(2,2,2-trifluoroethoxy)pyridin-2-amine (520 mg, 1.92 mmol, 72% yield). LCMS (ESI) m/z 270.8 (M+H)⁺, ¹H NMR (400 MHz, METHANOL-d₄) δ ppm 4.61 (q, J=8.28 Hz, 2H) 6.19-6.25 (m, 1H) 7.86-7.91 (m, 1H).

### Example 26: N-(2-cyclopropyl-7-(2,2,2-trifluoroethoxy)imidazo[1,2-a]pyridin-6-yl)-6-(difluoromethyl)picolinamide

N-(2-cyclopropyl-7-(2,2,2-trifluoroethoxy)imidazo[,2-a]pyridin-6-yl)-6-(difluoromethyl)picolinamide was prepared in a similar manner to that described for Example 5: Step b, substituting 5-bromo-4-(2,2,2-trifluoroethoxy)pyridin-2-amine (Intermediate 17) in place of N-(6-amino-4-(cyclobutylmethoxy)pyridin-3-yl)-6-(difluoromethyl)picolinamide. LCMS (ESI) m/z 427.1 (M+H)⁺, ¹H NMR (400 MHz, METHANOL-d₄) δ ppm 0.94-1.00 (m, 2H) 1.18-1.25 (m, 2H) 2.10-2.18 (m, 1H) 5.07 (q, J=8.03 Hz, 2H) 6.62-6.92 (m, 1H) 7.49 (s, 1H) 7.86 (s, 1H) 7.99 (d, J=7.78 Hz, 1H) 8.29 (t, J=7.78 Hz, 1H) 8.40 (d, J=7.78 Hz, 1H) 9.77 (s, 1H).

### Example 27: 6-(difluoromethyl)-N-(7-ethoxy-2-(3-hydroxy-3-methylbutyl)imidazo[1,2-a]pyridin-6-yl)picolinamide

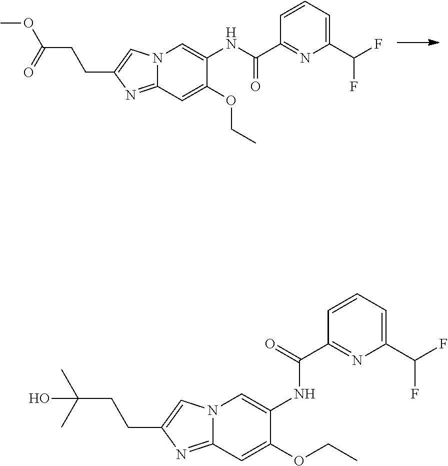 [SMILES: CCOc1cc2nc(CCC(=O)OC)cn2cc1NC(=O)c1cccc(C(F)F)n1 ; CCOc1cc2nc(CCC(C)(C)O)cn2cc1NC(=O)c1cccc(C(F)F)n1]

To a solution of methyl 3-(6-(6-(difluoromethyl)picolinamido)-7-ethoxyimidazo[1,2-a]pyridin-2-yl)propanoate from Example 23: Step a (87 mg, 208 μmol) in THF (2 mL) at 0° C. was added MeMgBr (3.0 M, 0.7 mL). After stirring for 4 h, the reaction mixture was quenched with saturated aqueous NHCl₄ solution. The layers were separated and the aqueous phase was extracted with EtOAc (3×10 mL), dried over MgSO₄, filtered, concentrated, and purified by TFA-modified mass-directed HPLC on a reverse phase column (5-95% gradient of water to acetonitrile) to obtain -(difluoromethyl)-N-(7-ethoxy-2-(3-hydroxy-3-methylbutyl)imidazo[1,2-a]pyridin-6-yl)picolinamide (39.6 mg, 94.6 μmol, 45% yield). LCMS (ESI) m/z 419.3 (M+H)+, ¹H NMR (400 MHz, METHANOL-d₄) δ ppm 1.27-1.33 (m, 6H) 1.61-1.67 (m, 3H) 1.88-1.95 (m, 2H) 2.88-2.95 (m, 2H) 4.38-4.45 (m, 2H) 6.68-6.98 (m, 1H) 7.25-7.29 (m, 1H) 7.77-7.81 (m, 1H) 7.93 (dd, J=7.78, 0.75 Hz, 1H) 8.24 (t, J=7.78 Hz, 1H) 8.34 (dd, J=7.78, 1.00 Hz, 1H) 9.65-9.69 (m, 1H).

### Example 28: N-(2-cyclopropyl-8-ethoxyimidazo[1,2-a]pyridin-6-yl)-6-(difluoromethyl)picolinamide

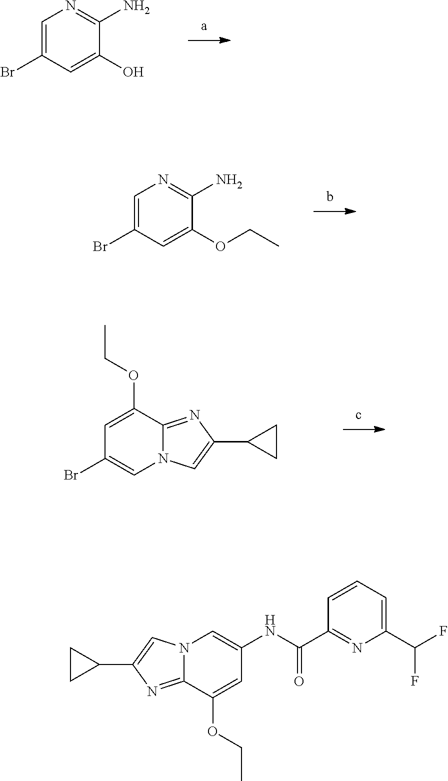 [SMILES: Nc1ncc(Br)cc1O ; CCOc1cc(Br)cnc1N ; CCOc1cc(Br)cn2cc(C3CC3)nc12 ; CCOc1cc(NC(=O)c2cccc(C(F)F)n2)cn2cc(C3CC3)nc12]

Step a: To a vial with a mixture of ethanol (536 mg, 11.6 mmol, 678 μL), triphenylphosphine (3.05 g, 11.6 mmol), 2-amino-5-bromo-pyridin-3-ol (2.00 g, 10.6 mmol) in THF (20 mL) was added diisopropyl azodicarboxylate (11.64 mmol, 2.3 mL) dropwise at 0° C. After addition, the reaction mixture was stirred at 22° C. for 16 h. MeOH and silica gel were added to the mixture, which was then concentrated and purified by silica gel chromatography (dry load onto silica gel, eluting with a 0-100% gradient of a 3:1 blend of EtOAc:EtOH in heptanes) to give 5-bromo-3-ethoxypyridin-2-amine (2.50 g, 11.5 mmol, 108% yield; mixed with triphenylphosphine oxide, used directly in the next step). LCMS (ESI) m/z 218.0 (M+H)⁺.

Step b: To a vial charged with 5-bromo-3-ethoxy-pyridin-2-amine (688 mg, 3.17 mmol), 2-bromo-1-cyclopropyl-ethanone (258 mg, 1.59 mmol) was added EtOH (9 mL), followed by sodium bicarbonate (798 mg, 9.51 mmol) as a solid in one portion, open to air. The vial was sealed and heated at 80° C. for 18 h. Then MeOH and silica gel were added to the mixture, which was then concentrated and purified by silica gel chromatography (dry load onto silica gel, eluting with a 0-100% gradient of a 3:1 blend of EtOAc:EtOH in heptanes) to provide 6-bromo-2-cyclopropyl-8-ethoxy-imidazo[1,2-a]pyridine (310 mg, 1.10 mmol, 34.8% yield). LCMS (ESI) m/z 321.1 (M+H)⁺.

Step c: A vial charged with 6-bromo-2-cyclopropyl-8-ethoxy-imidazo[1,2-a]pyridine (120 mg, 426 μmol), Pd(OAc)₂ (19.2 mg, 85.4 μmol), (5-diphenylphosphanyl-9,9-dimethyl-xanthen-4-yl)-diphenyl-phosphane (98 mg, 171 μmol), 6-(difluoromethyl)pyridine-2-carboxamide (184 mg, 1.07 mmol) and Cs₂CO₃ (278 mg, 853.64 μmol) was evacuated, backfilled with N₂ and closed with a screw cap with septa. Dioxane (3 mL) was added via syringe at 22° C. The vial was sealed and heated at 100° C. for 16 h. Then the reaction mixture was allowed to cool to room temperature, filtered through celite, concentrated and purified by TFA-modified mass-directed HPLC on a reverse phase column (5-95% gradient of water to acetonitrile) to afford N-(2-cyclopropyl-8-ethoxyimidazo[1,2-a]pyridin-6-yl)-6-(difluoromethyl)picolinamide (24 mg, 65 μmol, 15% yield). LCMS (ESI) m/z 391.3 (M+H)+, ¹H NMR (400 MHz, METHANOL-d₄) δ ppm 1.57 (t, J=6.90 Hz, 3H) 1.91-2.00 (m, 2H) 2.76 (t, J=7.65 Hz, 2H) 3.65 (t, J=6.53 Hz, 2H) 4.21 (q, J=6.86 Hz, 2H) 6.64-6.93 (m, 2H) 7.41 (s, 1H) 7.88 (d, J=7.78 Hz, 1H) 8.16-8.22 (m, 1H) 8.26-8.30 (m, 1H) 9.34 (s, 1H).

### Example 29: N-(2-cyclopropyl-7-ethoxy-3-fluoroimidazo[1,2-a]pyridin-6-yl)-6-(difluoromethyl)picolinamide

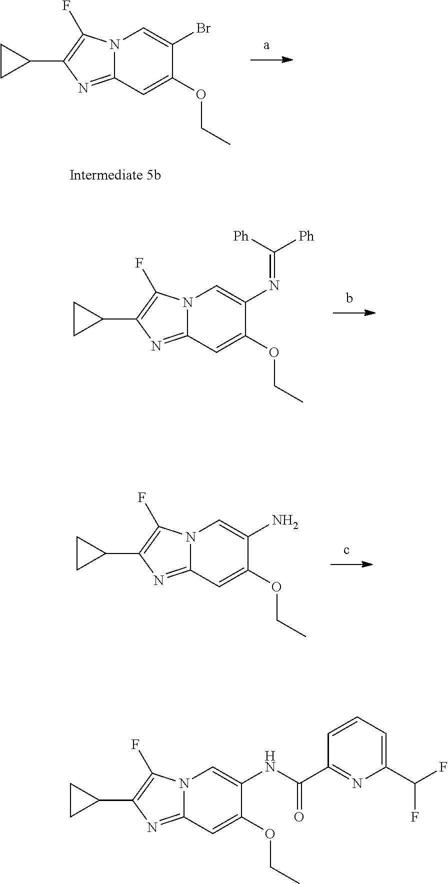 [SMILES: CCOc1cc2nc(C3CC3)c(F)n2cc1Br ; CCOc1cc2nc(C3CC3)c(F)n2cc1N=C(c1ccccc1)c1ccccc1 ; CCOc1cc2nc(C3CC3)c(F)n2cc1N ; CCOc1cc2nc(C3CC3)c(F)n2cc1NC(=O)c1cccc(C(F)F)n1]

N-(2-cyclopropyl-7-ethoxy-3-fluoroimidazo[1,2-a]pyridin-6-yl)-6-(difluoromethyl)picolinamide was prepared in a similar manner to that described for Example 4, starting from Intermediate 5b: 6-bromo-2-cyclopropyl-7-ethoxy-3-fluoroimidazo[1,2-a]pyridine. LCMS (ESI) m/z 391.2 (M+H)+, ¹H NMR (400 MHz, METHANOL-d₄) δ ppm 0.99-1.06 (m, 2H) 1.18-1.25 (m, 2H) 1.69 (t, J=6.90 Hz, 3H) 2.06-2.14 (m, 1H) 4.48 (q, J=6.86 Hz, 2H) 6.74-7.03 (m, 1H) 7.25 (d, J=1.25 Hz, 1H) 8.00 (d, J=7.78 Hz, 1H) 8.30 (t, J=7.78 Hz, 1H) 8.43 (dd, J=7.78, 0.75 Hz, 1H) 9.53 (s, 1H).

### Example 30: N-(2-cyclopropylimidazo[1,2-a]pyridin-6-yl)-6-(difluoromethyl)picolinamide

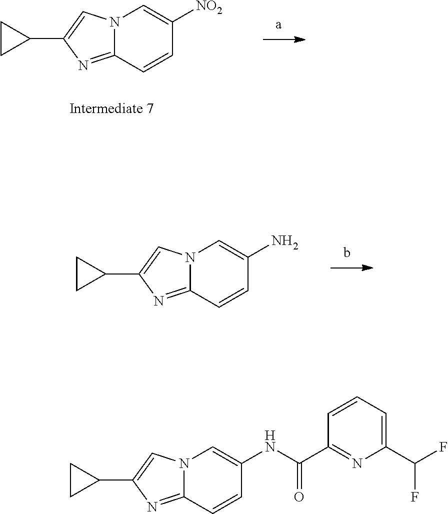 [SMILES: O=[N+]([O-])c1ccc2nc(C3CC3)cn2c1 ; Nc1ccc2nc(C3CC3)cn2c1 ; O=C(Nc1ccc2nc(C3CC3)cn2c1)c1cccc(C(F)F)n1]

Step a: To a solution of crude 2-cyclopropyl-6-nitroimidazo[1,2-a]pyridine, Intermediate 7: (300 mg, 1.48 mmol) in a 3:1 blend of EtOAc/EtOH (6 mL) was added palladium on carbon (39 mg, 10 wt %). The suspension was stirred at room temperature under 1 atmosphere of hydrogen (balloon) for 18 h. The mixture was then filtered through a celite pad, washing with MeOH (30 mL). The filtrate was concentrated in vacuo to provide 2-cyclopropylimidazo[1,2-a]pyridin-6-amine as a black solid which was used without further purification in the next reaction assuming 100% yield; LCMS (ESI) m/z 174.1 (M+H)+.

Step b: DIPEA (4.44 mmol, 0.8 mL) was added to a mixture of 2-cyclopropylimidazo[1,2-a]pyridin-6-amine (256 mg, 1.48 mmol), HATU (564 mg, 1.48 mmol) and 6-(difluoromethyl)pyridine-2-carboxylic acid (281 mg, 1.63 mmol) in DMF (4 mL) at 0° C. The reaction was warmed to rt and stirred for 12 h before being quenched with brine (50 mL), extracted with EtOAc (3×20 mL), washed with brine (3×10 mL), dried over MgSO₄, filtered, concentrated, and purified by silica gel chromatography eluting with a gradient of 0-70% of a 3:1 EtOAc:EtOH blend in heptanes to obtain N-(2-cyclopropylimidazo[1,2-a]pyridin-6-yl)-6-(difluoromethyl)picolinamide (264 mg, 804 μmol, 54% yield) as a pale yellow solid. LCMS (ESI) m/z 329.1 (M+H)+, ¹H NMR (400 MHz, CHLOROFORM-d) δ ppm 0.94-1.02 (m, 4H) 2.04 (tt, J=8.09, 5.33 Hz, 1H) 6.59-6.88 (m, 1H) 7.08-7.11 (m, 1H) 7.39 (s, 1H) 7.54 (d, J=9.54 Hz, 1H) 7.86 (d, J=7.53 Hz, 1H) 8.11 (t, J=7.78 Hz, 1H) 8.40 (dd, J=7.78, 0.75 Hz, 1H) 9.27 (d, J=1.25 Hz, 1H) 9.71 (s, 1H).

### Example 31: N-(2-cyclopropylimidazo[1,2-a]pyrazin-6-yl)-6-(difluoromethyl)picolinamide

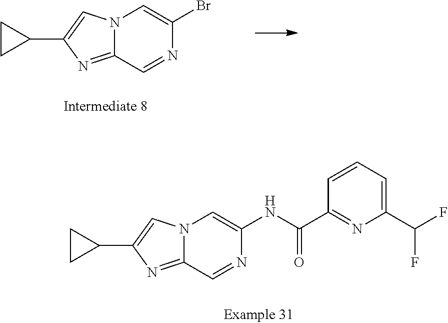 [SMILES: Brc1cn2cc(C3CC3)nc2cn1 ; O=C(Nc1cn2cc(C3CC3)nc2cn1)c1cccc(C(F)F)n1]

N-(2-cyclopropylimidazo[1,2-a]pyrazin-6-yl)-6-(difluoromethyl)picolinamide was prepared in a similar manner to that described for Example 5 (step c), starting from Intermediate 8: 6-bromo-2-cyclopropylimidazo[1,2-a]pyrazine. LCMS (ESI) m/z 330.1 (M+H)⁺, ¹H NMR (400 MHz, METHANOL-d₄) δ ppm 1.05-1.11 (m, 2H) 1.26-1.33 (m, 2H) 2.22-2.29 (m, 1H) 6.78-7.08 (m, 1H) 8.00 (d, J=7.28 Hz, 1H) 8.18 (s, 1H) 8.28 (t, J=7.78 Hz, 1H) 8.43 (dd, J=7.78, 0.75 Hz, 1H) 9.06-9.09 (m, 1H) 9.64 (d, J=1.51 Hz, 1H).

### Example 32: 6-acetamido-N-(7-ethoxy-2-ethylimidazo[1,2-a]pyridin-6-yl)picolinamide

To a solution of 6-acetamidopyridine-2-carboxylic acid (88 mg, 487 μmol) and DIPEA (63 mg, 487 μmol, 85 μL) in DMF (1 mL) is added 7-ethoxy-2-ethyl-imidazo[1,2-a]pyridin-6-amine (50 mg, 243 μmol) Then, T3P (310 mg, 487 μmol, 310 μL, 50% purity) is added dropwise with stirring and the reaction was maintained at rt for 16 h. Then, the reaction was quenched with water (10 mL), diluted with EtOAc (20 mL), and the layers separated. The aqueous layer was extracted with EtOAc (3×10 mL) and the combined organic layers were, concentrated. The resulting residue was taken up in 2 mL of DMSO and purified by reverse-phase HPLC (Gilson, column: SunFire Prep C18 OBD, 5 micrometers, 30×50 nM column) eluting with a %1 TFA modified gradient of 10%-90% MeCN gave 6-acetamido-N-(7-ethoxy-2-ethylimidazo[1,2-a]pyridin-6-yl)picolinamide (2.6 mg, 7.1 μmol, 2.9% yield). LCMS (ESI) m/z 368.1 (M+H)^{+ 1}H NMR (500 MHz, METHANOL-d₄) δ ppm 1.39-1.45 (m, 3H) 1.68 (t, J=7.02 Hz, 3H) 2.24-2.28 (m, 3H) 2.84-2.93 (m, 2H) 4.55 (q, J=6.71 Hz, 2H) 7.27-7.32 (m, 1H) 7.84 (s, 1H) 7.98-8.08 (m, 2H) 8.30 (d, J=7.94 Hz, 1H) 9.75-9.80 (m, 1H)

### Example 33: N-(2-(3,3-difluorocyclobutyl)-7-ethoxyimidazo[1,2-a]pyridin-6-yl)-6-(trifluoromethyl)picolinamide

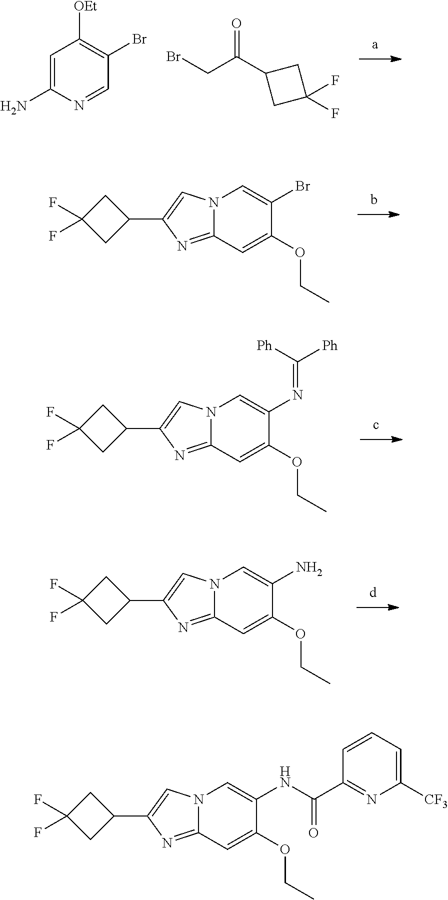

Step a: 5-bromo-4-ethoxy-pyridin-2-amine (254 mg, 1.17 mmol), 2-bromo-1-(3,3-difluorocyclobutyl)ethanone (250 mg, 1.17 mmol) were dissolved in EtOH (3 mL) and then sodium bicarbonate (295 mg, 3.51 mmol) was added. The vial was sealed and heated at 85° C. for 18 h. Silica was then added and the mixture was concentrated. The mixture was purified via column chromatography eluting with a gradient of 5-100% of a 3:1 blend of EtOAc:EtOH in heptanes to provide 6-bromo-2-(3,3-difluorocyclobutyl)-7-ethoxy-imidazo[1,2-a]pyridine (210 mg, 634 μmol, 54% yield). LCMS (ESI) m/z 331.3 (M+H)⁺.

Step b: A vial was charged with sodium tert-butoxide (85 mg, 887 μmol), Pd₂(dba)₃ (17 mg, 19 μmol), [1-(2-diphenylphosphanyl-1-naphthyl)-2-naphthyl]-diphenyl-phosphane (31 mg, 50 μmol) and 6-bromo-2-(3,3-difluorocyclobutyl)-7-ethoxyimidazo[1,2-a]pyridine (210 mg, 634 μmol). The vial was flushed with N₂, and toluene (1 mL), then diphenylmethanimine (137 mg, 761 μmol, 0.12 mL) were added. The vial was sealed, and the mixture heated at 100° C. overnight. The reaction was then concentrated onto silica gel and purified via silica gel column chromatography eluting with a gradient of 5-100% of a 3:1 blend of EtOAc:EtOH in heptanes to give N-[2-(3,3-difluorocyclobutyl)-7-ethoxy-imidazo[1,2-a]pyridin-6-yl]-1,1-diphenyl-methanimine (170 mg, 394 μmol, 62% yield) LCMS (ESI) m/z 432.5 (M+H)⁺.

Step c: N-[2-(3,3-difluorocyclobutyl)-7-ethoxy-imidazo[1,2-a]pyridin-6-yl]-1,1-diphenyl-methanimine (170 mg, 393 μmol) in DCM (4 mL) and MeOH (4 mL) was treated with HCl (4 M in Dioxane, 0.25 mL) and stirred for 1 hour. The reaction was then concentrated, then passed through a SCX column, eluting first with MeOH, and then 2 M ammonia in MeOH in to give 2-(3,3-difluorocyclobutyl)-7-ethoxy-imidazo[1,2-a]pyridin-6-amine (100 mg, 374 μmol, 95% yield). LCMS (ESI) m/z 268.4 (M+H)⁺.

Step d: A vial was charged with 6-(trifluoromethyl)pyridine-2-carboxylic acid (78 mg, 412 μmol), then 2-(3,3-difluorocyclobutyl)-7-ethoxy-imidazo[1,2-a]pyridin-6-amine (100 mg, 374 μmol) followed by DCM (1 mL). To this heterogeneous mixture was added DIPEA (145 mg, 1.12 mmol, 200 μL), followed by T3P (748 μmol, 340 μL, 50 wt % in EtOAc). The reaction was stirred overnight, then the reaction mixture was partitioned between 1 N NaOH (10 mL) and EtOAc (10 mL). The layers were separated and the aqueous layer was extracted with EtOAc (3×10 mL). The combined organic layers were dried (Na₂SO₄) and concentrated. The residue was purified via reverse phase HPLC (Gilson, column: SunFire Prep C18 OBD, 5 micrometers, 30×50 nM column) eluting with a %1 TFA modified gradient of 10%-90% MeCN to give N-(2-(3,3-difluorocyclobutyl)-7-ethoxyimidazo[1,2-a]pyridin-6-yl)-6-(trifluoromethyl)picolinamide (38 mg, 87 μmol, 23% yield). LCMS (ESI) m/z 441.5 (M+H)⁺. ¹H NMR (500 MHz, METHANOL-d₄) δ ppm 1.68 (t, J=7.02 Hz, 3H) 2.80-2.98 (m, 2H) 3.11-3.21 (m, 2H) 3.60-3.72 (m, 1H) 4.48 (q, J=7.33 Hz, 2H) 7.30 (s, 1H) 8.05 (s, 1H) 8.08-8.24 (m, 1H) 8.39 (t, J=7.94 Hz, 1H) 8.54 (d, J=7.94 Hz, 1H) 9.67-9.98 (m, 1H) 10.73 (s, 1H).

### Example 34: N-(7-ethoxy-2-ethylimidazo[1,2-a]pyridin-6-yl)-5,6-dimethylpicolinamide

7-ethoxy-2-ethyl-imidazo[1,2-a]pyridin-6-amine (25 mg, 122 μmol) and 5,6-dimethylpyridine-2-carboxylic acid (45 mg, 299 μmol) in DMF (1 mL) were treated with DIPEA (62 mg, 487 μmol, 85 μL), and BOP (108 mg, 243 μmol). The solution was maintained at rt overnight and was then directly purified via reverse phase HPLC (Gilson, column: SunFire Prep C18 OBD, 5 micrometers, 30×50 nM column) eluting with a %1 TFA modified gradient of 10%-90% MeCN to give N-(7-ethoxy-2-ethylimidazo[1,2-a]pyridin-6-yl)-5,6-dimethylpicolinamide (25 mg, 74 μmol, 61% yield). LCMS (ESI) m/z 339.5 (M+H)⁺. ¹H NMR (500 MHz, METHANOL-d₄) δ ppm 1.29-1.52 (m, 3H) 1.71 (t, J=7.02 Hz, 3H) 2.44 (s, 3H) 2.56-2.69 (s, 3H) 2.82-2.97 (m, 2H) 4.48 (q, J=6.71 Hz, 2H) 7.26 (s, 1H) 7.79 (d, J=7.94 Hz, 1H) 7.85 (s, 1H) 8.01 (d, J=7.94 Hz, 1H) 9.77 (s, 1H).

### Example 35: N-(7-ethoxy-2-ethylimidazo[1,2-a]pyridin-6-yl)-3,4-difluorobenzamide

Example 35: was prepared in a similar fashion to Example 34: with 3,4-difluorobenzoic acid replacing 5,6-dimethylpyridine-2-carboxylic acid to yield N-(7-ethoxy-2-ethylimidazo[1,2-a]pyridin-6-yl)-3,4-difluorobenzamide. ¹H NMR (500 MHz, METHANOL-d₄) δ ppm 1.41 (t, J=7.63 Hz, 3H) 1.59 (t, J=7.02 Hz, 3H) 2.82-2.95 (m, 2H) 4.42 (q, J=6.71 Hz, 2H) 7.26 (s, 1H) 7.50 (dt, J=10.38, 8.24 Hz, 1H) 7.81 (s, 1H) 7.84 (ddd, J=6.41, 4.27, 2.14 Hz, 1H) 7.93 (ddd, J=10.99, 7.63, 2.14 Hz, 1H) 9.42 (s, 1H).

### Example 36: N-(7-ethoxy-2-ethylimidazo[1,2-a]pyridin-6-yl)-3-fluoro-6-(trifluoromethyl)picolinamide

N-(7-ethoxy-2-ethylimidazo[1,2-a]pyridin-6-yl)-3-fluoro-6-(trifluoromethyl)picolinamide was prepared in a similar fashion to Example 34: with 3-fluoro-6-(trifluoromethyl)picolinic acid replacing 5,6-dimethylpyridine-2-carboxylic acid to yield N-(7-ethoxy-2-ethylimidazo[1,2-a]pyridin-6-yl)-3-fluoro-6-(trifluoromethyl)picolinamide. ¹H NMR (500 MHz, METHANOL-d₄) δ ppm 1.33-1.51 (m, 3H) 1.65 (t, J=7.02 Hz, 3H) 2.81-2.94 (m, 2H) 4.45 (q, J=6.71 Hz, 2H) 7.29 (s, 1H) 7.86 (s, 1H) 8.14 (t, J=9.16 Hz, 1H) 8.23 (dd, J=9.16, 3.05 Hz, 1H) 9.75 (s, 1H).

### Example 37: N-(7-ethoxy-2-ethylimidazo[1,2-a]pyridin-6-yl)-1,3-dihydroisobenzofuran-5-carboxamide

Example 37: was prepared in a similar fashion to Example 34: with 1,3-dihydroisobenzofuran-5-carboxylic acid replacing 5,6-dimethylpyridine-2-carboxylic acid to yield N-(7-ethoxy-2-ethylimidazo[1,2-a]pyridin-6-yl)-1,3-dihydroisobenzofuran-5-carboxamide. ¹H NMR (500 MHz, METHANOL-d₄) δ ppm 1.42 (t, J=7.33 Hz, 3H) 1.60 (t, J=6.71 Hz, 3H) 2.89 (q, J=7.53 Hz, 2 H) 4.43 (q, J=6.71 Hz, 2H) 5.17 (s, 4H) 7.26 (s, 1H) 7.49 (d, J=7.94 Hz, 1H) 7.82 (s, 1H) 7.87-7.94 (m, 2H) 9.48 (s, 1H)

### Example 38: N-(7-ethoxy-2-ethylimidazo[1,2-a]pyridin-6-yl)-5,6-dihydro-4H-pyrrolo[1,2-b]pyrazole-2-carboxamide

N-(7-ethoxy-2-ethylimidazo[1,2-a]pyridin-6-yl)-5,6-dihydro-4H-pyrrolo[1,2-b]pyrazole-2-carboxamide was prepared in a similar fashion to Example 34: with 5,6-dihydro-4H-pyrrolo[1,2-b]pyrazole-2-carboxylic acid replacing 5,6-dimethylpyridine-2-carboxylic acid to yield N-(7-ethoxy-2-ethylimidazo[1,2-a]pyridin-6-yl)-5,6-dihydro-4H-pyrrolo[1,2-b]pyrazole-2-carboxamide. ¹H NMR (500 MHz, METHANOL-d₄) δ ppm 1.31-1.51 (m, 3H) 1.56-1.69 (m, 3H) 2.70 (t, J=7.33 Hz, 2H) 2.83-2.91 (m, 2H) 2.99 (t, J=7.33 Hz, 2H) 4.19-4.29 (m, 2H) 4.46 (q, J=6.71 Hz, 2H) 6.61 (s, 1H) 7.26 (s, 1H) 7.82 (s, 1H) 9.66 (s, 1H).

### Example 39: N-(7-ethoxy-2-ethylimidazo[1,2-a]pyridin-6-yl)-4-methyl-3,4-dihydro-2H-benzo[b][1,4]oxazine-8-carboxamide

Example 39: was prepared in a similar fashion to Example 34: with 4-methyl-3,4-dihydro-2H-benzo[b][1,4]oxazine-8-carboxylic acid replacing 5,6-dimethylpyridine-2-carboxylic acid to yield N-(7-ethoxy-2-ethylimidazo[1,2-a]pyridin-6-yl)-4-methyl-3,4-dihydro-2H-benzo[b][1,4]oxazine-8-carboxamide. ¹H NMR (500 MHz, METHANOL-d₄) δ ppm 1.37-1.45 (m, 3H) 1.68 (t, J=7.02 Hz, 3H) 2.83-2.92 (m, 2H) 2.96 (s, 3H) 3.38-3.49 (m, 2H) 4.43 (q, J=7.33 Hz, 2H) 4.54-4.68 (m, 2H) 6.90-7.06 (m, 2H) 7.21 (s, 1H) 7.48 (dd, J=7.32, 1.83 Hz, 1H) 7.81 (s, 1H) 9.83 (s, 1H)

### Example 40: N-(7-ethoxy-2-ethylimidazo[1,2-a]pyridin-6-yl)-2,4-difluorobenzamide

Example 40 was prepared in a similar fashion to Example 34: with 2,4-difluorobenzoic acid replacing 5,6-dimethylpyridine-2-carboxylic acid to yield N-(7-ethoxy-2-ethylimidazo[1,2-a]pyridin-6-yl)-2,4-difluorobenzamide. ¹H NMR (500 MHz, METHANOL-d₄) δ ppm 1.41 (t, J=7.63 Hz, 3H) 1.62 (t, J=7.02 Hz, 3H) 2.84-2.98 (m, 2H) 4.43 (q, J=7.12 Hz, 2H) 7.17-7.34 (m, 3H) 7.83 (s, 1H) 8.16 (td, J=8.85, 6.71 Hz, 1H) 9.71 (s, 1H)

### Example 41: N-(7-ethoxy-2-ethylimidazo[1,2-a]pyridin-6-yl)-2-oxoindoline-6-carboxamide

Example 41 was prepared in a similar fashion to Example 34: with 2-oxoindoline-6-carboxylic acid replacing 5,6-dimethylpyridine-2-carboxylic acid to yield N-(7-ethoxy-2-ethylimidazo[1,2-a]pyridin-6-yl)-2-oxoindoline-6-carboxamide. LCMS (ESI) m/z 365.1 (M+H)⁺.

### Example 42: N-(7-ethoxy-2-ethylimidazo[1,2-a]pyridin-6-yl)-2,3-dihydrobenzofuran-6-carboxamide

Example 42 was prepared in a similar fashion to Example 34: with 2,3-dihydrobenzofuran-6-carboxylic acid replacing 5,6-dimethylpyridine-2-carboxylic acid to yield N-(7-ethoxy-2-ethylimidazo[1,2-a]pyridin-6-yl)-2,3-dihydrobenzofuran-6-carboxamide ¹H NMR (500 MHz, METHANOL-d₄) δ ppm 1.41 (t, J=7.63 Hz, 3H) 1.60 (t, J=6.71 Hz, 3H) 2.82-2.93 (m, 2H) 3.32 (s, 2H) 4.42 (q, J=7.32 Hz, 2H) 4.65 (t, J=8.55 Hz, 2H) 7.25 (s, 1H) 7.29 (d, J=1.22 Hz, 1H) 7.39 (d, J=7.94 Hz, 1H) 7.44-7.49 (m, 1H) 7.81 (s, 1H) 9.45 (s, 1H)

### Example 43: N-(7-ethoxy-2-ethylimidazo[1,2-a]pyridin-6-yl)isochromane-7-carboxamide

Example 43 was prepared in a similar fashion to Example 34: with isochromane-7-carboxylic acid replacing 5,6-dimethylpyridine-2-carboxylic acid to yield N-(7-ethoxy-2-ethylimidazo[1,2-a]pyridin-6-yl)isochromane-7-carboxamide. ¹H NMR (500 MHz, METHANOL-d₄) δ ppm 1.37-1.49 (m, 3H) 1.54-1.67 (m, 3H) 2.83-2.92 (m, 2H) 2.96 (t, J=5.49 Hz, 2H) 4.02 (t, J=5.80 Hz, 2H) 4.42 (q, J=6.71 Hz, 2H) 4.85 (s, 2H) 7.25 (s, 1H) 7.35 (d, J=7.94 Hz, 1H) 7.66 (s, 1H) 7.77 (dd, J=7.94, 1.83 Hz, 1H) 7.81 (s, 1H) 9.45 (s, 1H).

### Example 44: 1-(difluoromethyl)-N-(7-ethoxy-2-ethylimidazo[1,2-a]pyridin-6-yl)-1H-pyrazole-3-carboxamide

Example 44 was prepared in a similar fashion to Example 34: with 1-(difluoromethyl)-1H-pyrazole-3-carboxylic acid replacing 5,6-dimethylpyridine-2-carboxylic acid to yield 1-(difluoromethyl)-N-(7-ethoxy-2-ethylimidazo[1,2-a]pyridin-6-yl)-1H-pyrazole-3-carboxamide. ¹H NMR (500 MHz, METHANOL-d₄) δ ppm 1.33-1.48 (m, 3H) 1.63 (t, J=7.02 Hz, 3H) 2.83-2.95 (m, 2H) 4.46 (q, J=6.92 Hz, 2H) 7.09 (d, J=2.44 Hz, 1H) 7.27 (s, 1H) 7.53-7.81 (m, 1H) 7.84 (s, 1H) 8.27 (d, J=2.44 Hz, 1H) 9.65 (s, 1H).

### Example 45: N-(7-ethoxy-2-ethylimidazo[1,2-a]pyridin-6-yl)-4,5-dimethylpicolinamide

Example 45 was prepared in a similar fashion to Example 34: with 4,5-dimethylpicolinic acid replacing 5,6-dimethylpyridine-2-carboxylic acid to yield N-(7-ethoxy-2-ethylimidazo[1,2-a]pyridin-6-yl)-4,5-dimethylpicolinamide. ¹H NMR (500 MHz, METHANOL-d₄) δ ppm 1.36-1.46 (m, 3H) 1.66 (t, J=7.02 Hz, 3H) 2.40 (s, 3H) 2.44 (s, 3H) 2.81-2.93 (m, 2H) 4.47 (q, J=6.71 Hz, 2H) 7.26 (s, 1H) 7.83 (s, 1H) 8.03 (s, 1H) 8.43 (s, 1H) 9.76 (s, 1H).

### Example 46: 5-chloro-N-(7-ethoxy-2-ethylimidazo[1,2-a]pyridin-6-yl)picolinamide

Example 46 was prepared in a similar fashion to Example 34: with 5-chloropicolinic acid replacing 5,6-dimethylpyridine-2-carboxylic acid to yield 5-chloro-N-(7-ethoxy-2-ethylimidazo[1,2-a]pyridin-6-yl)picolinamide. ¹H NMR (500 MHz, METHANOL-d₄) δ ppm 1.34-1.47 (m, 3H) 1.66 (t, J=7.02 Hz, 3H) 2.80-2.94 (m, 2H) 4.48 (q, J=6.71 Hz, 2H) 7.29 (s, 1H) 7.84 (s, 1H) 8.14 (dd, J=8.55, 2.44 Hz, 1H) 8.25-8.30 (m, 1H) 8.74 (d, J=2.44 Hz, 1H) 9.77 (s, 1H)

### Example 47: N-(7-ethoxy-2-ethylimidazo[1,2-a]pyridin-6-yl)isochromane-6-carboxamide

Example 47 was prepared in a similar fashion to Example 34: with isochromane-6-carboxylic acid replacing 5,6-dimethylpyridine-2-carboxylic acid to yield N-(7-ethoxy-2-ethylimidazo[1,2-a]pyridin-6-yl)isochromane-6-carboxamide. ¹H NMR (500 MHz, METHANOL-d₄) δ ppm 1.36-1.47 (m, 3H) 1.60 (t, J=7.02 Hz, 3H) 2.82-2.93 (m, 2H) 2.97 (t, J=5.49 Hz, 2H) 4.02 (t, J=5.80 Hz, 2H) 4.42 (q, J=7.32 Hz, 2H) 4.84 (s, 2H) 7.19-7.28 (m, 2H) 7.73-7.85 (m, 3H) 9.46 (s, 1H).

### Example 48: N-(7-ethoxy-2-ethylimidazo[1,2-a]pyridin-6-yl)-4,5-dimethylthiazole-2-carboxamide

Example 48 was prepared in a similar fashion to Example 34: with 4,5-dimethylthiazole-2-carboxylic acid replacing 5,6-dimethylpyridine-2-carboxylic acid to yield N-(7-ethoxy-2-ethylimidazo[1,2-a]pyridin-6-yl)-4,5-dimethylthiazole-2-carboxamide. ¹H NMR (500 MHz, METHANOL-d₄) δ ppm 1.33-1.47 (m, 3H) 1.64 (t, J=7.02 Hz, 3H) 2.44 (s, 3H) 2.51 (s, 3H) 2.82-2.93 (m, 2H) 4.47 (q, J=6.71 Hz, 2H) 7.28 (s, 1H) 7.83 (s, 1H) 9.63 (s, 1H).

### Example 49: N-(7-ethoxy-2-ethylimidazo[1,2-a]pyridin-6-yl)-6,7-dihydro-5H-cyclopenta[b]pyridine-2-carboxamide

Example 49 was prepared in a similar fashion to Example 34: with 6,7-dihydro-5H-cyclopenta[b]pyridine-2-carboxylic acid replacing 5,6-dimethylpyridine-2-carboxylic acid to yield N-(7-ethoxy-2-ethylimidazo[1,2-a]pyridin-6-yl)-6,7-dihydro-5H-cyclopenta[b]pyridine-2-carboxamide. LCMS (ESI) m/z 351.1 (M+H)⁺

### Example 50: N-(2-cyclopropyl-7-((3-methyloxetan-3-yl)methoxy)imidazo[1,2-a]pyridin-6-yl)-6-(difluoromethyl)picolinamide

Example 50 N-(2-cyclopropyl-7-((3-methyloxetan-3-yl)methoxy)imidazo[1,2-a]pyridin-6-yl)-6-(difluoromethyl)picolinamide was prepared through the same synthetic sequence as described for the preparation of Example 5: except for substituting (3-methyloxetan-3-yl)methanol in place of cyclobutylmethanol in Step a. LCMS (ESI) m/z 429.2 (M+H)⁺. ¹H NMR (400 MHz, METHANOL-d4) δ ppm 9.29 (s, 1H), 8.19 (dd, J=0.75, 7.78 Hz, 1H), 8.09 (t, J=7.78 Hz, 1H), 7.80 (dd, J=0.88, 7.66 Hz, 1H), 7.30 (s, 1H), 6.66-6.98 (m, 2H), 4.83 (d, J=6.02 Hz, 2H), 4.53 (d, J=6.27 Hz, 2H), 4.13 (s, 2H), 1.87-1.97 (m, 1H), 1.55 (s, 3H), 0.91-1.00 (m, 2H), 0.78-0.86 (m, 2H).

### Example 51: N-(7-ethoxy-2-ethylimidazo[1,2-a]pyridin-6-yl)-5-fluoro-6-(trifluoromethyl)picolinamide

Example 51 was prepared in a similar fashion to Example 34: with 5-fluoro-6-(trifluoromethyl)picolinic acid replacing 5,6-dimethylpyridine-2-carboxylic acid to yield N-(7-ethoxy-2-ethylimidazo[1,2-a]pyridin-6-yl)-5-fluoro-6-(trifluoromethyl)picolinamide after additional purification using the base-modified reverse phase preparative HPLC conditions: Waters XSelect CSH Prep C18 5 um OBD 19×100 mm, modifier was ammonium hydroxide water (0.04% NH4OH+10 mM NH4HCO3)-acetonitrile; Gradient used 10-70%. ¹H NMR (500 MHz, METHANOL-d₄) δ ppm 1.33 (t, J=7.63 Hz, 3H) 1.59 (t, J=7.02 Hz, 3H) 2.70-2.80 (m, 2H) 4.27 (q, J=6.92 Hz, 2H) 6.89 (s, 1H) 7.46 (s, 1H) 8.13 (t, J=9.46 Hz, 1H) 8.54 (dd, J=8.55, 3.66 Hz, 1H) 9.43 (s, 1H).

### Example 52: N-(2-cyclopropyl-7-methoxyimidazo[1,2-a]pyridin-6-yl)-1-methyl-2-oxo-1,2-dihydropyridine-3-carboxamide

Example 52 was prepared in a similar fashion to Example 34: with 1-methyl-2-oxo-1,2-dihydropyridine-3-carboxylic acid replacing 5,6-dimethylpyridine-2-carboxylic acid and 2-cyclopropyl-7-methoxyimidazo[1,2-a]pyridin-6-amine replacing Intermediate 5: 7-ethoxy-2-ethylimidazo[1,2-a]pyridin-6-amine to yield N-(2-cyclopropyl-7-methoxyimidazo[1,2-a]pyridin-6-yl)-1-methyl-2-oxo-1,2-dihydropyridine-3-carboxamide. ¹H NMR (500 MHz, METHANOL-d₄) δ ppm 0.87-0.99 (m, 2H) 1.13-1.20 (m, 2H) 2.04-2.16 (m, 1H) 3.74 (s, 3H) 4.23 (s, 3H) 6.59-6.70 (m, 1H) 7.21 (s, 1H) 7.75 (s, 1H) 8.06 (dd, J=6.41, 2.14 Hz, 1H) 8.59 (dd, J=7.32, 2.44 Hz, 1H) 9.77 (s, 1H).

### Example 53: N-(2-cyclopropyl-7-ethoxyimidazo[1,2-a]pyridin-6-yl)pyrazolo[1,5-a]pyrimidine-2-carboxamide

Example 53 was prepared in a similar fashion to Example 34: with pyrazolo[1,5-a]pyrimidine-2-carboxylic acid replacing 5,6-dimethylpyridine-2-carboxylic acid and Intermediate 5c: 2-cyclopropyl-7-ethoxyimidazo[1,2-a]pyridin-6-amine replacing Intermediate 5: 7-ethoxy-2-ethylimidazo[1,2-a]pyridin-6-amine to yield N-(2-cyclopropyl-7-ethoxyimidazo[1,2-a]pyridin-6-yl)pyrazolo[1,5-a]pyrimidine-2-carboxamide. LCMS (ESI) m/z 363.5 (M+H)⁺. ¹H NMR (500 MHz, METHANOL-d₄) δ ppm 0.87-1.00 (m, 2H) 1.14-1.25 (m, 2H) 1.67 (t, J=6.71 Hz, 3H) 2.04-2.16 (m, 1H) 4.47 (q, J=7.33 Hz, 2H) 7.20 (dd, J=7.02, 3.97 Hz, 1H) 7.24 (d, J=9.16 Hz, 2H) 7.77 (s, 1H) 8.65 (dd, J=4.27, 1.83 Hz, 1H) 9.01-9.08 (m, 1H) 9.66 (s, 1H).

### Example 54: N-(2-cyclopropyl-7-ethoxyimidazo[1,2-a]pyridin-6-yl)-3-(trifluoromethyl)benzamide

Example 54 was prepared in a similar fashion to Example 34: with 3-(trifluoromethyl)benzoic acid replacing pyrazolo[1,5-a]pyrimidine-2-carboxylic acid. LCMS (ESI) m/z 391.5 (M+H)⁺. ¹H NMR (500 MHz, METHANOL-d₄) δ ppm 0.91-1.01 (m, 2H) 1.13-1.34 (m, 2H) 1.59 (t, J=7.02 Hz, 3H) 2.06-2.19 (m, 1H) 4.41 (q, J=7.12 Hz, 2H) 7.22 (s, 1H) 7.77 (s, 1H) 7.80 (t, J=7.63 Hz, 1H) 7.97 (d, J=7.94 Hz, 1H) 8.23 (d, J=7.94 Hz, 1H) 8.28 (s, 1H) 9.40 (s, 1H)

### Example 55: N-(7-ethoxy-2-(ethoxymethyl)imidazo[1,2-a]pyridin-6-yl)-6-(trifluoromethyl)picolinamide

Example 55 A vial was charged with Intermediate 4: N-(6-amino-4-ethoxy-3-pyridyl)-6-(trifluoromethyl)picolinamide (499 mg, 1.53 mmol), 1,3-dichloropropan-2-one (194 mg, 1.53 mmol, 140 μL) and EtOH (4 mL) followed by sodium bicarbonate (385 mg, 4.6 mmol). The vial was sealed at heated at 85° C. for 18 h. The reaction was then filtered, and the solvent was removed. The mixture was dried and loaded into a dry loading column. The residue was purified via reverse phase HPLC (Gilson, column: SunFire Prep C18 OBD, 5 micrometers, 30×50 nM column) eluting with a %1 TFA modified gradient of 10%-90% MeCN to give to give N-(7-ethoxy-2-(ethoxymethyl)imidazo[1,2-a]pyridin-6-yl)-6-(trifluoromethyl)picolinamide (1.8 mg, 4.4 μmol). LCMS (ESI) m/z 409.5 (M+H)⁺. ¹H NMR (500 MHz, METHANOL-d₄) δ ppm 1.29 (t, J=7.02 Hz, 3H) 1.67 (t, J=7.02 Hz, 3H) 3.68 (q, J=7.33 Hz, 2H) 4.46 (q, J=6.71 Hz, 2H) 4.65-4.76 (m, 2H) 7.23-7.41 (m, 1H) 8.05 (s, 1H) 8.14 (d, J=7.94 Hz, 1H) 8.38 (t, J=7.94 Hz, 1H) 8.52 (d, J=7.94 Hz, 1H) 9.78-9.94 (m, 1H).

### Example 56: N-(7-ethoxy-2-(methoxymethyl)imidazo[1,2-a]pyridin-6-yl)-6-(trifluoromethyl)picolinamide

Example 56 was prepared in a similar fashion as Example 55: except MeOH was used as the solvent, and the solution was only heated to 60° C. LCMS (ESI) m/z 395.5 (M+H)⁺. ¹H NMR (500 MHz, METHANOL-d₄) δ ppm 1.68 (t, J=7.02 Hz, 4H) 3.49 (s, 3H) 4.47 (q, J=6.71 Hz, 3H) 4.68 (s, 2H) 7.31 (s, 1H) 8.06 (s, 1H) 8.14 (d, J=7.94 Hz, 1H) 8.38 (t, J=7.94 Hz, 1H) 8.53 (d, J=7.33 Hz, 1H) 9.82 (s, 1H)

### Example 57: N-(2-(difluoromethyl)-7-ethoxyimidazo[1,2-a]pyridin-6-yl)-6-(trifluoromethyl)picolinamide

Example 57 was prepared in a similar fashion as Example 55: except 3-bromo-1,1-difluoropropan-2-one was used in place of 1,3-dichloropropan-2-one. LCMS (ESI) m/z 401.5 (M+H)⁺. ¹H NMR (500 MHz, METHANOL-d₄) δ ppm 1.68 (t, J=7.02 Hz, 3H) 4.46 (q, J=6.71 Hz, 2H) 7.01-7.35 (m, 2H) 8.14 (dd, J=7.94, 1.22 Hz, 1H) 8.34-8.42 (m, 2H) 8.53 (d, J=7.94 Hz, 1H) 9.81-9.85 (m, 1H)

### Example 58: N-(7-ethoxy-2,3-dimethylimidazo[1,2-a]pyridin-6-yl)-6-(trifluoromethyl)picolinamide

Example 58 was prepared in a similar fashion as Example 55: except 3-bromobutan-2-one was used in place of 1,3-dichloropropan-2-one. LCMS (ESI) m/z 379.4 (M+H)⁺. ¹H NMR (500 MHz, METHANOL-d₄) δ ppm 1.65-1.71 (m, 3H) 2.48 (s, 3H) 2.55 (s, 3H) 4.46 (q, J=6.71 Hz, 2H) 7.32 (s, 1H) 8.16 (s, 1H) 8.14 (s, 1H) 8.39 (t, J=7.94 Hz, 1H) 8.54 (br d, J=7.33 Hz, 1H) 9.53 (s, 1H)

### Example 59: N-(7-ethoxy-2-(tetrahydrofuran-3-yl)imidazo[1,2-a]pyridin-6-yl)-6-(trifluoromethyl)picolinamide

Example 59 was prepared in a similar fashion as Example 55, except 2-bromo-1-tetrahydrofuran-3-yl-ethanone was used in place of 1,3-dichloropropan-2-one. LCMS (ESI) m/z 421.5 (M+H)⁺.

### Example 60: N-(7-ethoxy-2-((tetrahydrofuran-2-yl)methyl)imidazo[1,2-a]pyridin-6-yl)-6-(trifluoromethyl)picolinamide

Example 60 was prepared in a similar fashion as Example 55, except 1-bromo-3-(tetrahydrofuran-2-yl)propan-2-one was used in place of 1,3-dichloropropan-2-one. LCMS (ESI) m/z 435.5 (M+H)⁺. ¹H NMR (500 MHz, METHANOL-d₄) δ ppm 1.61 (t, J=7.02 Hz, 3H) 1.65-1.76 (m, 1H) 1.88-2.01 (m, 2H) 2.01-2.11 (m, 1H) 2.82-2.91 (m, 1H) 2.98 (dd, J=14.65, 6.71 Hz, 1H) 3.71-3.84 (m, 1H) 3.89-4.01 (m, 1H) 4.20-4.37 (m, 3H) 6.94 (s, 1H) 7.57 (s, 1H) 8.10 (d, J=8.55 Hz, 1H) 8.34 (t, J=7.63 Hz, 1H) 8.49 (d, J=7.94 Hz, 1H) 9.36-9.55 (m, 1H).

### Example 61: N-(2-(azetidin-3-yl)-7-ethoxyimidazo[1,2-a]pyridin-6-yl)-6-(trifluoromethyl)picolinamide

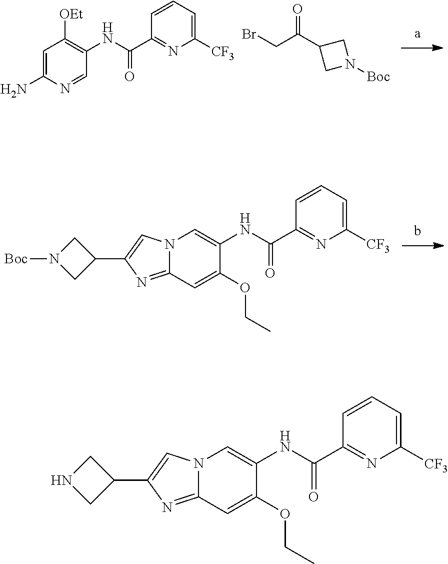 [SMILES: CCOc1cc(N)ncc1NC(=O)c1cccc(C(F)(F)F)n1 ; CC(C)(C)OC(=O)N1CC(C(=O)CBr)C1 ; CCOc1cc2nc(C3CN(C(=O)OC(C)(C)C)C3)cn2cc1NC(=O)c1cccc(C(F)(F)F)n1 ; CCOc1cc2nc(C3CNC3)cn2cc1NC(=O)c1cccc(C(F)(F)F)n1]

Step a: A vial was charged with Intermediate 4: N-(6-amino-4-ethoxy-3-pyridyl)-6-(trifluoromethyl)picolinamide (1.17 g, 3.60 mmol), sodium bicarbonate (907 mg, 10.8 mmol) and EtOH (9 mL) followed by tert-butyl 3-(2-bromoacetyl)azetidine-1-carboxylate (1.00 g, 3.60 mmol). The vial was sealed and heated at 85° C. for 18 h. Silica was added, and the mixture was dried and loaded into a dry loading column. The residue was then eluted through a 40 g Reveleris 40 micron silica gel column (Grace X2 automated purification machine) using a gradient of 5%-100% of a 3:1 EtOAc:EtOH blend in heptanes to provide tert-butyl 3-[7-ethoxy-6-[[6-(trifluoromethyl)pyridine-2-carbonyl]amino]imidazo[1,2-a]pyridin-2-yl]azetidine-1-carboxylate (260 mg, 514 μmol, 14% yield). LCMS (ESI) m/z 506.6 (M+H)⁺.

Step b: tert-butyl 3-[7-ethoxy-6-[[6-(trifluoromethyl)pyridine-2-carbonyl]amino]imidazo[1,2-a]pyridin-2-yl]azetidine-1-carboxylate (260 mg, 514 μmol) was dissolved in HCl (4 M in Dioxane, 20 mL) and the solution was stirred at room temperature for 2.5 h. The solution was concentrated, and a portion was purified via reverse phase HPLC to give Example 61: N-(2-(azetidin-3-yl)-7-ethoxyimidazo[1,2-a]pyridin-6-yl)-6-(trifluoromethyl)picolinamide (7.6 mg, 14.66 μmol). LCMS (ESI) m/z 406.4 (M+H)⁺. ¹H NMR (500 MHz, METHANOL-d₄) δ ppm 1.68 (t, J=7.02 Hz, 3H) 4.35-4.62 (m, 7H) 7.45 (s, 1H) 8.15 (t, J=3.97 Hz, 2H) 8.39 (t, J=7.94 Hz, 1H) 8.54 (d, J=7.94 Hz, 1H) 9.81-9.84 (m, 1H)

### Example 62: N-(7-ethoxy-2-(pyrrolidin-3-yl)imidazo[1,2-a]pyridin-6-yl)-6-(trifluoromethyl)picolinamide

Example 62 was prepared in a similar fashion as Example 61: except tert-butyl 3-(2-bromoacetyl)pyrrolidine-1-carboxylate was used in place of tert-butyl 3-(2-bromoacetyl)azetidine-1-carboxylate. LCMS (ESI) m/z 420.5 (M+H)⁺. ¹H NMR (500 MHz, METHANOL-d4) δ ppm 1.68 (t, J=7.02 Hz, 3H) 2.33 (dq, J=12.82, 8.55 Hz, 1H) 2.59-2.73 (m, 1H) 3.45-3.56 (m, 2H) 3.62 (ddd, J=12.21, 7.94, 4.27 Hz, 1H) 3.83-3.99 (m, 2H) 4.48 (q, J=6.71 Hz, 2H) 7.42 (s, 1H) 8.07 (s, 1H) 8.12-8.20 (m, 1H) 8.38 (t, J=7.94 Hz, 1H) 8.53 (d, J=7.94 Hz, 1H) 9.77-9.86 (m, 1H).

### Example 63: N-(7-ethoxy-2-(piperidin-4-yl)imidazo[1,2-a]pyridin-6-yl)-6-(trifluoromethyl)picolinamide

Example 63 was prepared in a similar fashion as Example 61: except tert-butyl 4-(2-bromoacetyl)piperidine-1-carboxylate was used in place of tert-butyl 3-(2-bromoacetyl)azetidine-1-carboxylate. LCMS (ESI) m/z 465.5 (M+Na)⁺. ¹H NMR (500 MHz, METHANOL-d₄) δ ppm 1.68 (t, J=7.02 Hz, 3H) 1.97-2.11 (m, 2H) 2.39 (br d, J=13.43 Hz, 2H) 3.24 (td, J=12.97, 2.75 Hz, 2H) 3.58 (br d, J=12.82 Hz, 2H) 4.47 (q, J=6.71 Hz, 2H) 7.41 (s, 1H) 7.99 (s, 1H) 8.15 (d, J=7.94 Hz, 1H) 8.38 (t, J=7.94 Hz, 1H) 8.53 (d, J=7.33 Hz, 1H) 9.81 (s, 1H).

### Example 64: N-(7-ethoxy-1,2,3,4-tetrahydroimidazo[1,2-a:5,4-c′]dipyridin-8-yl)-6-(trifluoromethyl)picolinamide

Example 64 was prepared in a similar fashion as Example 61: except tert-butyl 3-bromo-4-oxopiperidine-1-carboxylate was used in place of tert-butyl 3-(2-bromoacetyl)azetidine-1-carboxylate. LCMS (ESI) m/z 406.5 (M+H)⁺. ¹H NMR (500 MHz, METHANOL-d₄) δ ppm 1.68 (t, J=7.02 Hz, 3H) 3.27 (t, J=6.10 Hz, 2H) 3.75 (t, J=6.10 Hz, 2H) 4.49 (q, J=6.92 Hz, 2H) 4.75 (s, 2H) 7.48 (s, 1H) 8.16 (d, J=7.94 Hz, 1H) 8.37-8.41 (m, 1H) 8.54 (d, J=7.94 Hz, 1H) 9.50-9.56 (m, 1H)

### Example 65: N-(7-ethoxy-2-(1-methylazetidin-3-yl)imidazo[1,2-a]pyridin-6-yl)-6-(trifluoromethyl)picolinamide

Example 61: N-(2-(azetidin-3-yl)-7-ethoxyimidazo[1,2-a]pyridin-6-yl)-6-(trifluoromethyl)picolinamide (60 mg, 148 μmol) was dissolved in methanol (1 mL), and formaldehyde (14 mg, 177 μmol, 37% purity) and acetic acid (18 mg, 296 μmol, 20 μL) were added. The solution was stirred for 30 min before sodium cyanoborohydride (28 mg, 444 μmol) was added and the resulting mixture was stirred at rt overnight. The mixture was diluted with EtOAc (10 mL), washed with water (10 mL), dried, and concentrated in vacuo. The resulting residue was purified via prep HPLC to give N-(7-ethoxy-2-(1-methylazetidin-3-yl)imidazo[1,2-a]pyridin-6-yl)-6-(trifluoromethyl)picolinamide (20 mg, 37 μmol, 25% yield). LCMS (ESI) m/z 420.5 (M+H)⁺. ¹H NMR (500 MHz, METHANOL-d₄) δ ppm 1.68 (t, J=7.02 Hz, 3H) 3.06-3.15 (m, 3H) 4.25-4.78 (m, 7H) 7.48-7.54 (m, 1H) 8.08-8.21 (m, 2H) 8.38 (t, J=7.94 Hz, 1H) 8.53 (d, J=7.94 Hz, 1H) 9.81 (s, 1H).

### Example 66: N-(7-ethoxy-2-(1-methylpyrrolidin-3-yl)imidazo[1,2-a]pyridin-6-yl)-6-(trifluoromethyl)picolinamide

Example 66 was prepared in a similar fashion as Example 65: except Example 62 was used in place of Example 61: LCMS (ESI) m/z 434.5 (M+H)⁺. ¹H NMR (500 MHz, METHANOL-d₄) δ ppm 1.68 (t, J=7.02 Hz, 3H) 2.42 (br s, 1H) 2.74 (br s, 1H) 3.08 (s, 3H) 3.40-4.21 (m, 5H) 4.49 (q, J=7.12 Hz, 2H) 7.42 (s, 1H) 8.08 (s, 1H) 8.15 (d, J=7.94 Hz, 1H) 8.39 (t, J=7.94 Hz, 1H) 8.54 (d, J=7.94 Hz, 1H) 9.80-9.84 (m, 1H)

### Example 67: N-(7-ethoxy-2-(1-methylpiperidin-4-yl)imidazo[1,2-a]pyridin-6-yl)-6-(trifluoromethyl)picolinamide

Example 67 was prepared in a similar fashion as Example 65: except Example 63 was used in place of Example 61: LCMS (ESI) m/z 448.6 (M+H)⁺. ¹H NMR (500 MHz, METHANOL-d₄) δ ppm 1.68 (t, J=7.02 Hz, 3H) 2.10 (br dd, J=13.12, 3.36 Hz, 2H) 2.44 (br d, J=14.65 Hz, 2H) 2.97 (s, 3H) 3.20-3.30 (m, 3H) 3.72 (br d, J=12.21 Hz, 2H) 4.47 (q, J=6.71 Hz, 2H) 7.41 (s, 1H) 7.99 (s, 1H) 8.14 (d, J=7.94 Hz, 1H) 8.38 (t, J=7.94 Hz, 1H) 8.53 (d, J=7.94 Hz, 1H) 9.80 (s, 1H).

### Example 68: N-(2-(1-acetylazetidin-3-yl)-7-ethoxyimidazo[1,2-a]pyridin-6-yl)-6-(trifluoromethyl)picolinamide

N-(2-(azetidin-3-yl)-7-ethoxyimidazo[1,2-a]pyridin-6-yl)-6-(trifluoromethyl)picolinamide (62 mg, 151 μmol) in DMF (1 mL) was treated with DIPEA (49 mg, 380 μmol, 0.8 mL), then acetyl chloride (14 mg, 182 μmol, 13 μL). The resulting solution was stirred overnight at rt. Then the solution was diluted with a minimal amount of MeOH, and then purified directly via reverse-phase TFA modified preparative HPLC, to give N-(2-(1-acetylazetidin-3-yl)-7-ethoxyimidazo[1,2-a]pyridin-6-yl)-6-(trifluoromethyl)picolinamide (16 mg, 28 μmol, 19% yield). LCMS (ESI) m/z 448.5 (M+H)⁺. ¹H NMR (500 MHz, METHANOL-d₄) δ ppm 1.68 (t, J=7.02 Hz, 3H) 1.96 (s, 3H) 4.10-4.25 (m, 2H) 4.41 (dd, J=8.55, 5.49 Hz, 1H) 4.48 (q, J=6.92 Hz, 3H) 4.68-4.78 (m, 1H) 7.36 (s, 1H) 8.11 (s, 1H) 8.14 (d, J=7.32 Hz, 1H) 8.38 (t, J=7.63 Hz, 1H) 8.53 (d, J=7.94 Hz, 1H) 9.82 (s, 1H).

### Example 69: N-(2-(1-acetylpyrrolidin-3-yl)-7-ethoxyimidazo[1,2-a]pyridin-6-yl)-6-(trifluoromethyl)picolinamide

Example 69 was prepared in a similar fashion as Example 68: except Example 62 was used in place of Example 61: LCMS (ESI) m/z 462.5 (M+H)⁺. ¹H NMR (500 MHz, METHANOL-d₄) δ ppm 1.67 (t, J=7.02 Hz, 3H) 2.14 (d, J=7.94 Hz, 3H) 2.19-2.37 (m, 1H) 2.44-2.64 (m, 1H) 3.53-3.64 (m, 1H) 3.73 (ddd, J=10.84, 8.09, 2.44 Hz, 2H) 3.77-3.88 (m, 1H) 3.97-4.17 (m, 1H) 4.39-4.53 (m, 2H) 7.35 (d, J=12.21 Hz, 1H) 8.02 (d, J=14.65 Hz, 1H) 8.13 (d, J=7.94 Hz, 1H) 8.37 (t, J=7.94 Hz, 1H) 8.50 (d, J=7.94 Hz, 1H) 9.77 (d, J=4.27 Hz, 1H)

### Example 70 and 71: N-(2-cyclopropyl-7-ethoxyimidazo[1,2-a]pyridin-6-yl)-2-(2,2,2-trifluoroethoxy)nicotinamide and N-(2-cyclopropyl-7-ethoxyimidazo[1,2-a]pyridin-6-yl)-2-oxo-1-(2,2,2-trifluoroethyl)-1,2-dihydropyridine-3-carboxamide

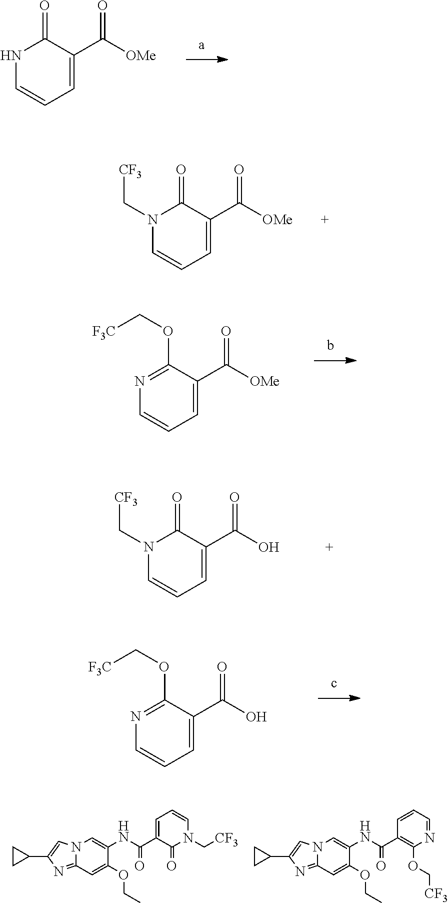

Step a: methyl 2-oxo-1H-pyridine-3-carboxylate (499 mg, 3.26 mmol) and Cs₂CO₃ (2.12 g, 6.52 mmol) in DMF (11 mL) was treated with 2,2,2-trifluoroethyl trifluoromethanesulfonate (832 mg, 3.59 mmol, 520 μL) and stirred for 5 hours at room temperature. The solution was then diluted with brine (200 mL), and extracted with EtOAc (2×100 mL). The combined organic layers were concentrated to provide a mixture of methyl 2-oxo-1-(2,2,2-trifluoroethyl)-1,2-dihydropyridine-3-carboxylate and methyl 2-(2,2,2-trifluoroethoxy)nicotinate. This mixture was not purified further, and was used in the next step without further manipulation assuming quantitative yield. LCMS (ESI) m/z 236.3 (M+H)⁺.

Step b: To a solution of methyl 2-oxo-1-(2,2,2-trifluoroethyl)pyridine-3-carboxylate and methyl 2-(2,2,2-trifluoroethoxy)nicotinate from Step a (766 mg, 3.26 mmol) in MeOH (4 mL), H₂O (4 mL), and THF (4 mL) was added LiOH (390 mg, 16.3 mmol). The mixture was stirred at 25° C. for 16 h. The reaction mixture was then concentrated and the residue was adjusted with HCl (1 M) to pH=3 and extracted with EtOAc (3×200 mL). The combined organic layers were washed with brine (100 mL), dried over Na₂SO₄, and filtered. The filtrate was concentrated to give the corresponding carboxylic acids, 2-oxo-1-(2,2,2-trifluoroethyl)-1,2-dihydropyridine-3-carboxylic acid and 2-(2,2,2-trifluoroethoxy)nicotinic acid as a mixture of regioisomers (41 mg, 185 μmol). This mixture was taken forward into the next step without further manipulation. LCMS (ESI) m/z 222.2 (M+H)⁺.

Step c: 2-cyclopropyl-7-ethoxy-imidazo[1,2-a]pyridin-6-amine (48 mg, 222 μmol) the regiomeric mixture from Step b (2-oxo-1-(2,2,2-trifluoroethyl)-1,2-dihydropyridine-3-carboxylic acid and 2-(2,2,2-trifluoroethoxy)nicotinic acid) (41 mg, 185.41 μmol) in DMF (1.8 mL) were treated with DIPEA (95 mg, 741 μmol, 0.13 mL), followed by BOP (164 mg, 371 μmol). The solution was maintained overnight at room temperature. The material was then directly purified via TFA-modified reverse phase preparative HPLC to give Example 70: N-(2-cyclopropyl-7-ethoxy-imidazo[1,2-a]pyridin-6-yl)-2-oxo-1-(2,2,2-trifluoroethyl)pyridine-3-carboxamide (21.80 mg, 40.87 μmol, 22.04% yield, Trifluoroacetate) and Example 71: N-(2-cyclopropyl-7-ethoxy-imidazo[1,2-a]pyridin-6-yl)-2-(2,2,2-trifluoroethoxy)pyridine-3-carboxamide (7.1 mg, 13 μmol, 7.2% yield). Data summarized below:

### Example 70: N-(2-cyclopropyl-7-ethoxyimidazo[1,2-a]pyridin-6-yl)-2-oxo-1-(2,2,2-trifluoroethyl)-1,2-dihydropyridine-3-carboxamide

LCMS (ESI) m/z 421.5 (M+H)⁺. ¹H NMR (500 MHz, METHANOL-d₄) δ ppm 0.89-0.96 (m, 2H) 1.15-1.21 (m, 2H) 1.63-1.68 (m, 3H) 2.05-2.14 (m, 1H) 4.42 (q, J=7.33 Hz, 2H) 5.01 (q, J=8.55 Hz, 2H) 6.69 (t, J=7.02 Hz, 1H) 7.18 (s, 1H) 7.73 (s, 1H) 8.03 (dd, J=6.71, 1.22 Hz, 1H) 8.63 (dd, J=7.32, 2.44 Hz, 1H) 9.73 (s, 1H).

### Example 71: N-(2-cyclopropyl-7-ethoxyimidazo[1,2-a]pyridin-6-yl)-2-(2,2,2-trifluoroethoxy)nicotinamide

LCMS (ESI) m/z 421.5 (M+H)⁺. ¹H NMR (500 MHz, METHANOL-d₄) δ ppm 0.91-0.99 (m, 2H) 1.16-1.24 (m, 2H) 1.61 (t, J=7.02 Hz, 3H) 2.07-2.16 (m, 1H) 4.52 (q, J=6.71 Hz, 2H) 5.32 (q, J=8.55 Hz, 2H) 7.25 (s, 1H) 7.32-7.42 (m, 1H) 7.79 (s, 1H) 8.42-8.51 (m, 1H) 8.56-8.70 (m, 1H) 9.83 (s, 1H).

### Example 72: N-(2-(1-acetylpiperidin-4-yl)-7-ethoxyimidazo[1,2-a]pyridin-6-yl)-6-(trifluoromethyl)picolinamide

Example 72 was prepared in a similar fashion as Example 68: except Example 63 was used in place of Example 61: LCMS (ESI) m/z 476.2 (M+H)⁺. ¹H NMR (500 MHz, METHANOL-d₄) δ ppm 1.67 (t, J=6.71 Hz, 5H) 2.10-2.30 (m, 5H) 2.82-2.93 (m, 1H) 3.17-3.23 (m, 1H) 3.35 (br d, J=1.22 Hz, 1H) 4.10 (br d, J=14.04 Hz, 1H) 4.46 (q, J=6.92 Hz, 2H) 4.67 (br d, J=14.04 Hz, 1H) 7.31 (s, 1H) 7.92 (s, 1H) 8.07-8.17 (m, 1H) 8.38 (t, J=7.63 Hz, 1H) 8.52 (d, J=7.94 Hz, 1H) 9.76-9.84 (m, 1H).

### Example 73: (S)—N-(2-cyclopropyl-7-(2-(5-oxopyrrolidin-2-yl)ethoxy)imidazo[1,2-a]pyridin-6-yl)-6-(difluoromethyl)picolinamide

Example 73 (S)—N-(2-cyclopropyl-7-(2-(5-oxopyrrolidin-2-yl)ethoxy)imidazo[1,2-a]pyridin-6-yl)-6-(difluoromethyl)picolinamide, was prepared through the same synthetic sequence as described for the preparation of Example 5, except for substituting (5S)-5-(2-hydroxyethyl)pyrrolidin-2-one in place of cyclobutylmethanol in step a. LCMS (ESI) m/z 456.2 (M+H)⁺. ¹H NMR (400 MHz, METHANOL-d4) δ ppm 0.89-1.00 (m, 2H), 1.15-1.27 (m, 2H), 1.91-2.04 (m, 1H), 2.07-2.18 (m, 1H), 2.23-2.31 (m, 2H), 2.34-2.53 (m, 3H), 4.15 (quin, J=6.65 Hz, 1H), 4.45-4.56 (m, 2H), 6.91 (t, J=56.0 Hz, 1H), 7.30 (s, 1H), 7.82 (s, 1H), 7.98 (d, J=7.78 Hz, 1H), 8.29 (t, J=7.78 Hz, 1H), 8.43 (dd, J=7.78, 0.75 Hz, 1H), 9.75 (s, 1H).

### Example 74: 6-(difluoromethyl)-N-(7-ethoxy-2-ethylimidazo[1,2-a]pyrimidin-6-yl)picolinamide

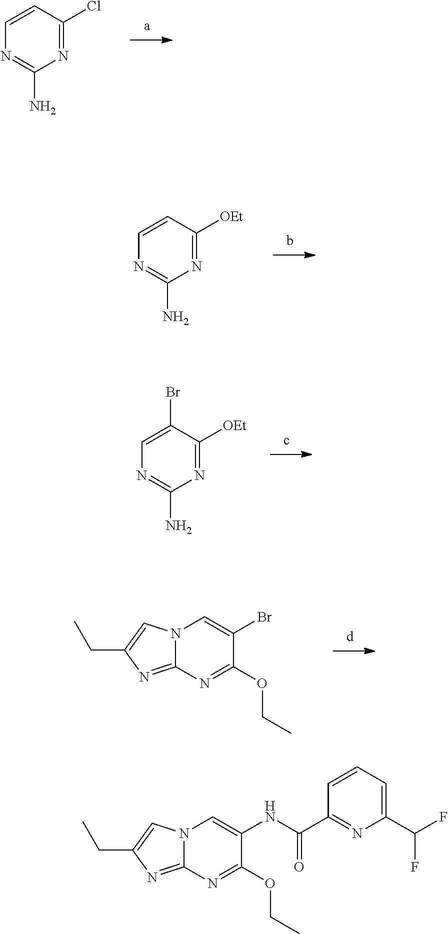 [SMILES: Nc1nccc(Cl)n1 ; CCOc1ccnc(N)n1 ; CCOc1nc(N)ncc1Br ; CCOc1nc2nc(CC)cn2cc1Br ; CCOc1nc2nc(CC)cn2cc1NC(=O)c1cccc(C(F)F)n1]

Step a: 4-chloropyrimidin-2-amine (2.00 g, 15.4 mmol) in EtOH (150 mL) was treated with sodium ethoxide (12.5 g, 38.6 mmol, 21 wt % in EtOH) and stirred at rt overnight. The pH of the solution was adjusted neutral with HCl, and the solution was concentrated to give 4-ethoxypyrimidin-2-amine, which was used without further purification assuming quantitative yield. LCMS (ESI) m/z 140.2 (M+H)⁺.

Step b: 4-ethoxypyrimidin-2-amine (2.15 g, 15.5 mmol) in chloroform (206 mL) was treated with NBS (2.75 g, 15.5 mmol) and stirred overnight in the dark. The solution was washed with sat. NaHCO₃, the organic layer was then dried and concentrated to give 5-bromo-4-ethoxy-pyrimidin-2-amine, which was used without further purification assuming quantitative yield. LCMS (ESI) m/z 218.2 (M+H)⁺.

Step c: A vial was charged with 5-bromo-4-ethoxy-pyrimidin-2-amine (1.40 g, 6.42 mmol) and 1-bromobutan-2-one (1.07 g, 7.06 mmol, 721 μL) followed by EtOH (16 mL) and sodium bicarbonate (1.62 g, 19.26 mmol). The vial was sealed at heated at 85° C. for 18 h. The reaction was transferred to a 30 mL vial and silica was added. The mixture was dried, and the resulting powder was then purified via silica gel column chromatography (gradient: 5-100% 3:1 EtOAc:EtOH blend in heptanes) to provide 6-bromo-7-ethoxy-2-ethyl-imidazo[1,2-a]pyrimidine (300 mg, 1.11 mmol, 17% yield), as a 3:1 mixture of regioisomers. LCMS (ESI) m/z 272.3 (M+H)⁺.

Step d: A vial charged with 6-bromo-7-ethoxy-2-ethyl-imidazo[1,2-a]pyrimidine (70 mg, 261 μmol), Pd(OAc)₂ (11 mg, 52 μmol), (5-diphenylphosphanyl-9,9-dimethyl-xanthen-4-yl)-diphenyl-phosphane (60 mg, 104 μmol), Cs₂CO₃ (170 mg, 522 μmol) and 6-(difluoromethyl)pyridine-2-carboxamide (90 mg, 523 μmol) was sealed with a septa cap and purged with N₂. Dioxane (1 mL) was added at rt and the vial was sealed with parafilm and heated at 100° C. for 16 h. The reaction was then cooled to rt and passed through a SCX column.

The filtrate was concentrated, and purified by TFA modified reverse-phase preparative HPLC to obtain 6-(difluoromethyl)-N-(7-ethoxy-2-ethyl-imidazo[1,2-a]pyrimidin-6-yl)pyridine-2-carboxamide (1.6 mg, 3.4 μmol, 1.3% yield) as a single regioisomer. LCMS (ESI) m/z 362.5 (M+H)⁺; ¹H NMR (500 MHz, METHANOL-d₄) δ ppm 1.27 (t, J=7.63 Hz, 3H) 1.51 (t, J=7.02 Hz, 3H) 2.66-2.79 (m, 2H) 4.64 (q, J=7.33 Hz, 2H) 6.63-6.98 (m, 1H) 7.56 (s, 1H) 7.88 (d, J=7.33 Hz, 1H) 8.17 (t, J=7.63 Hz, 1H) 8.28-8.33 (m, 1H) 9.71 (s, 1H).

### Example 75: 6-(difluoromethyl)-N-(7-ethoxy-2-(1-methyl-2-oxopiperidin-4-yl)imidazo[1,2-a]pyridin-6-yl)picolinamide

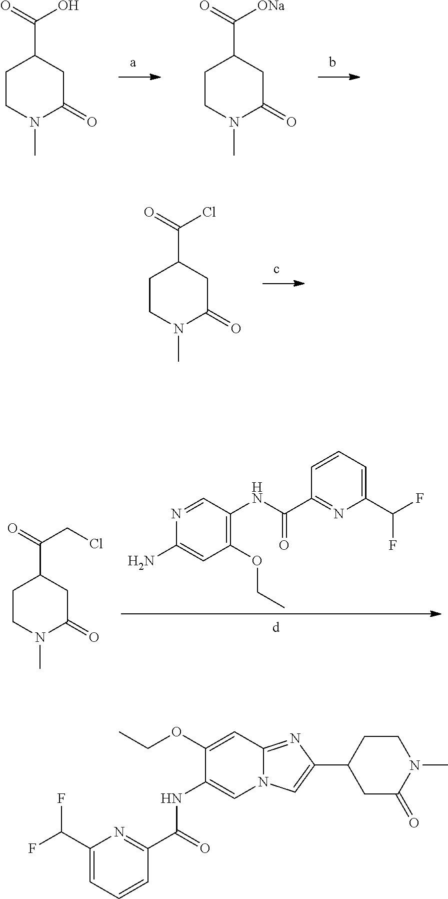

Step a: 1-methyl-2-oxopiperidine-4-carboxylic acid (500 mg, 3.18 mmol) and NaHCO₃ (267 mg, 3.2 mmol) were dissolved in water (10 mL). The solution was evaporated under reduced pressure to yield wet salt which was dried at 70° C. in high vacuo for 5 h to yield sodium 1-methyl-2-oxopiperidine-4-carboxylate (565 mg), which was used without further manipulation.

Step b: To a vial charged with sodium 1-methyl-2-oxopiperidine-4-carboxylate (565 mg, 3.1 mmol) and DCM (20 mL), was added oxalyl chloride (544 mg, 4.29 mmol). The resulting solution was maintained at room temperature for 20 h at which time it was concentrated in vacuo to provide 1-methyl-2-oxopiperidine-4-carbonyl chloride, which was used directly in the next step assuming quantitative yield.

Step c: 1-methyl-2-oxopiperidine-4-carbonyl chloride (3 mmol) was taken up in MeCN (20 mL) and THF solution of diazomethyl(trimethyl)silane (2M, 2.2 mL) was added and the mixture was kept at room temperature for 20 h. Aqueous hydrochloric acid (15%, 2 eq) was added and the mixture was stirred for 4 hours at which time, saturated aqueous NaHCO₃ was added portion-wise until pH-7. The organic phase was separated, concentrated under reduced pressure and distilled at 20 mBar (b.p. 120° C.) to yield 4-(2-chloroacetyl)-1-methylpiperidin-2-one (440 mg, 63% yield). 1H NMR (500 MHz, Chloroform-d) (500 MHz, DMSO-d6) 3.67 (d, J=3.4 Hz, 2H), 2.87 (dd, J=6.9, 3.6 Hz, 2H), 2.47-2.48 (m, 4H), 1.95-2.24 (m, 2H), 1.59-1.74 (m, 1H), 1.44 (m, 1H).

Step d: A vial was charged with 4-(2-chloroacetyl)-1-methylpiperidin-2-one (278 mg, 1.56 mmol), Intermediate 3c: N-(6-amino-4-ethoxypyridin-3-yl)-6-(difluoromethyl)picolinamide (Intermediate 3c) (462 mg, 1.20 mmol) and propionitrile. The resulting solution was heated at reflux for 20 hours. An aqueous solution of Na₂CO₃ (5 mL) was added and the mixture was stirred for 3 h. The organic phase was separated, concentrated under in vacuo, and purified by HPLC to provide 6-(difluoromethyl)-N-(7-ethoxy-2-(1-methyl-2-oxopiperidin-4-yl)imidazo[1,2-a]pyridin-6-yl)picolinamide (47 mg, 15% yield) as a racemic mixture. LCMS (ESI) m/z 362.5 (M+H)⁺; 1H NMR: (400 MHz, DMSO-d6) 10.48 (s, 1H), 9.42 (s, 1H), 8.34 (d, J=7.7 Hz, 1H), 8.26 (t, J=7.7 Hz, 1H), 7.93 (d, J=7.7 Hz, 1H), 7.52 (s, 1H), 6.96 (s, 1H), 6.86 (t, J=56 Hz, 1H), 4.25 (q, J=6.9 Hz, 2H), 3.11-3.49 (m, 3H), 2.87 (s, 3H), 2.48-2.60 (m, 2H), 2.03-2.19 (m, 2H), 1.59 (t, J=6.9 Hz, 3H).

### Example 76: 6-(difluoromethyl)-N-(2-ethyl-8-fluoroimidazo[1,2-a]pyridin-6-yl)picolinamide

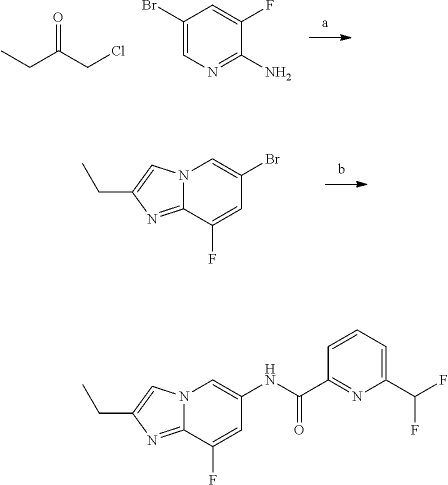

Step a: A vial was charged with 5-bromo-3-fluoro-pyridin-2-amine (1.00 g, 5.24 mmol), 1-chlorobutan-2-one (558 mg, 5.24 mmol), and EtOH (13 mL) followed by sodium bicarbonate (1.32 g, 15.7 mmol). The vial was sealed at heated at 85° C. for 18 h. A catalytic amount of NaI was added and the solution heated for an additional 24 h. Silica was then added, and the mixture was dried and purified via silica gel chromatography (gradient 5-100% 3:1 EtOAc:EtOH blend in heptanes) to provide 6-bromo-2-ethyl-8-fluoro-imidazo[1,2-a]pyridine (260 mg, 1.07 mmol, 20% yield). LCMS (ESI) m/z 243.2 (M+H)⁺.

Step b: A vial was charged with 6-bromo-2-ethyl-8-fluoro-imidazo[1,2-a]pyridine (260 mg, 1.07 mmol), Pd(OAc)₂ (48 mg, 214 μmol), (5-diphenylphosphanyl-9,9-dimethyl-xanthen-4-yl)-diphenyl-phosphane (247 mg, 428 μmol), Cs₂CO₃ (697 mg, 2.14 mmol) and 6-(difluoromethyl)pyridine-2-carboxamide (368 mg, 2.14 mmol). The vial was sealed with a septa cap and purged with N2. Dioxane (4 mL) was added at rt and the mixture was heated at 100° C. for 16 h. The mixture was cooled to rt, filtered through Celite, concentrated, and purified by TFA-modified mass-directed preparative reverse phase HPLC to obtain 6-(difluoromethyl)-N-(2-ethyl-8-fluoro-imidazo[1,2-a]pyridin-6-yl)pyridine-2-carboxamide (88 mg, 197 μmol, 18% yield). ¹H NMR (500 MHz, METHANOL-d₄) δ ppm 1.45 (t, J=7.63 Hz, 3H) 2.93-3.02 (m, 2H) 6.78-7.06 (m, 1H) 7.99 (d, J=7.94 Hz, 1H) 8.15-8.20 (m, 1H) 8.21-8.31 (m, 2H) 8.38-8.46 (m, 1H) 9.58 (d, J=1.83 Hz, 1H).

### Intermediate 9: N-(2-(azetidin-3-yl)-7-ethoxyimidazo[1,2-a]pyridin-6-yl)-6-(difluoromethyl)picolinamide

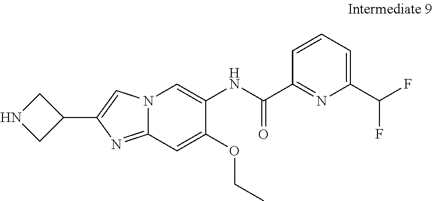 [SMILES: CCOc1cc2nc(C3CNC3)cn2cc1NC(=O)c1cccc(C(F)F)n1]

Intermediate 9: was prepared in a similar fashion as Example 61, except Intermediate 3b: 6-(difluoromethyl)picolinic acid was used in place of 6-(trifluoromethyl)picolinic acid. LCMS (ESI) m/z 388.5 (M+H)⁺.

### Example 77: 6-(difluoromethyl)-N-(7-ethoxy-2-(1-(2,2,2-trifluoroethyl)azetidin-3-yl)imidazo[1,2-a]pyridin-6-yl)picolinamide

N-(2-(azetidin-3-yl)-7-ethoxyimidazo[1,2-a]pyridin-6-yl)-6-(difluoromethyl)picolinamide (100 mg, 258 μmol) and Cs₂CO₃ (210 mg, 645 μmol) in DMF (1 mL) was treated with 2,2,2-trifluoroethyl trifluoromethanesulfonate (48 mg, 206 μmol, 30 μL) and stirred 5 h. The solution was then filtered, and then purified via TFA-modified reverse phase preparative HPLC to give 6-(difluoromethyl)-N-[7-ethoxy-2-[1-(2,2,2-trifluoroethyl)azetidin-3-yl]imidazo[1,2-a]pyridin-6-yl]pyridine-2-carboxamide (32 mg, 54 μmol, 21% yield) LCMS (ESI) m/z 470.5 (M+H) H NMR (500 MHz, METHANOL-d₄) δ ppm 1.68 (t, J=7.02 Hz, 3H) 3.81 (q, J=9.16 Hz, 2H) 4.09-4.14 (m, 2H) 4.20-4.29 (m, 1H) 4.29-4.38 (m, 2H) 4.46 (q, J=6.71 Hz, 2H) 6.73-7.01 (m, 1H) 7.43 (s, 1H) 7.97 (d, J=7.94 Hz, 1H) 8.07 (s, 1H) 8.27 (t, J=7.94 Hz, 1H) 8.39 (d, J=7.94 Hz, 1H) 9.77 (s, 1H)

### Example 78: N-(2-(1-(2,2-difluoroethyl)azetidin-3-yl)-7-ethoxyimidazo[1,2-a]pyridin-6-yl)-6-(difluoromethyl)picolinamide

Example 78 was prepared in a similar fashion as Example 77, except 2,2-difluoroethyl 4-methylbenzenesulfonate was used in place of 2,2,2-trifluoroethyl trifluoromethanesulfonate. LCMS (ESI) m/z 452.6 (M+H)⁺. ¹H NMR (500 MHz, METHANOL-d₄) δ ppm 1.69 (t, J=7.02 Hz, 3H) 3.78 (td, J=15.87, 3.05 Hz, 2H) 4.35-4.45 (m, 2H) 4.48 (q, J=7.12 Hz, 2H) 4.53-4.62 (m, 2H) 6.10-6.41 (m, 1H) 6.75-7.04 (m, 1H) 7.44 (s, 1H) 7.99 (d, J=7.94 Hz, 1H) 8.10 (s, 1H) 8.29 (t, J=7.63 Hz, 1H) 8.42 (d, J=7.32 Hz, 1H) 9.80 (s, 1H)

### Example 79: 6-(difluoromethyl)-N-(7-ethoxy-2-(1-(oxetan-3-yl)azetidin-3-yl)imidazo[1,2-a]pyridin-6-yl)picolinamide

N-(2-(azetidin-3-yl)-7-ethoxyimidazo[1,2-a]pyridin-6-yl)-6-(difluoromethyl)picolinamide (100 mg, 258 μmol) and Cs₂CO₃ (210 mg, 645 μmol) were dissolved in DMF (0.86 mL) and 3-bromooxetane (28 mg, 206 μmol, 17 μL) was added. The solution was heated to 70 C for a week, and then filtered. Purification via reverse phase HPLC gave 6-(difluoromethyl)-N-[7-ethoxy-2-[1-(oxetan-3-yl)azetidin-3-yl]imidazo[1,2-a]pyridin-6-yl]pyridine-2-carboxamide (1.5 mg, 3.4 μmol, 1.3% yield). LCMS (ESI) m/z 444.6 (M+H)⁺. ¹H NMR (500 MHz, METHANOL-d₄) δ ppm 1.62 (t, J=7.02 Hz, 3H) 3.43-3.54 (m, 2H) 3.77-3.98 (m, 4H) 4.30 (q, J=6.71 Hz, 2H) 4.58 (dd, J=6.71, 5.49 Hz, 2H) 4.79 (t, J=6.71 Hz, 2H) 6.72-6.99 (m, 2H) 7.66 (s, 1H) 7.95 (d, J=7.32 Hz, 1H) 8.26 (t, J=7.94 Hz, 1H) 8.38 (d, J=7.94 Hz, 1H) 9.48 (s, 1H)

### Example 80: 6-(difluoromethyl)-N-(7-ethoxy-2-(1-(2-methoxyethyl)azetidin-3-yl)imidazo[1,2-a]pyridin-6-yl)picolinamide

Example 80 was prepared in a similar fashion as Example 79, except 1-bromo-2-methoxyethane was used in place 3-bromooxetane. LCMS (ESI) m/z 446.6 (M+H)⁺. ¹H NMR (500 MHz, METHANOL-d₄) δ ppm 1.69 (t, J=7.02 Hz, 3H) 3.43 (s, 3H) 3.58-3.66 (m, 2H) 3.66-3.74 (m, 2H) 4.49 (q, J=6.71 Hz, 5H) 4.61-4.71 (m, 2H) 6.74-7.08 (m, 1H) 7.49 (s, 1H) 7.99 (d, J=7.94 Hz, 1H) 8.15 (s, 1H) 8.29 (t, J=7.63 Hz, 1H) 8.42 (d, J=7.33 Hz, 1H) 9.81 (s, 1H)

### Example 81: N-(2-(1-acetylazetidin-3-yl)-7-ethoxyimidazo[1,2-a]pyridin-6-yl)-6-(difluoromethyl)picolinamide

Example 81 was prepared in a similar fashion as Example 68, except N-(2-(azetidin-3-yl)-7-ethoxyimidazo[1,2-a]pyridin-6-yl)-6-(difluoromethyl)picolinamide was used in place N-(2-(azetidin-3-yl)-7-ethoxyimidazo[1,2-a]pyridin-6-yl)-6-(trifluoromethyl)picolinamide. LCMS (ESI) m/z 430.3 (M+H)⁺. ¹¹H NMR (500 MHz, METHANOL-d₄) δ ppm 1.69 (t, J=7.02 Hz, 3H) 1.96 (s, 3H) 4.12-4.26 (m, 2H) 4.39-4.52 (m, 4H) 4.68-4.77 (m, 1H) 6.67-7.10 (m, 1H) 7.37 (s, 1H) 7.98 (d, J=7.94 Hz, 1H) 8.09 (s, 1H) 8.28 (t, J=7.63 Hz, 1H) 8.40 (d, J=7.94 Hz, 1H) 9.79 (s, 1H)

### Example 82: N-(2-((1H-1,2,4-triazol-1-yl)methyl)-7-ethoxyimidazo[1,2-a]pyridin-6-yl)-6-(difluoromethyl)picolinamide

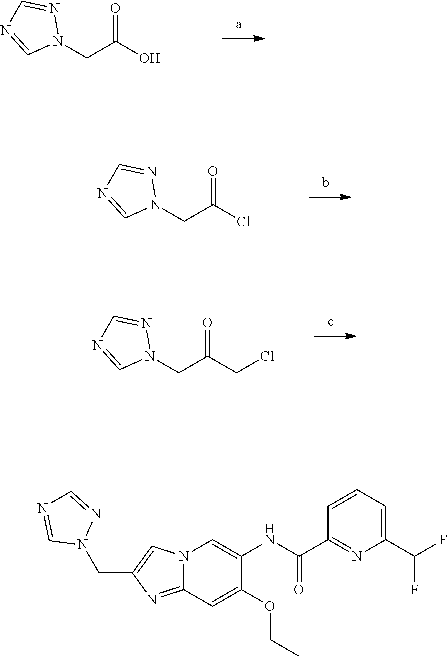 [SMILES: O=C(O)Cn1cncn1 ; O=C(Cl)Cn1cncn1 ; O=C(CCl)Cn1cncn1 ; CCOc1cc2nc(Cn3cncn3)cn2cc1NC(=O)c1cccc(C(F)F)n1]

Step a: 2-(1,2,4-triazol-1-yl)acetic acid (250 mg, 2.00 mmol) in DCM (3 mL) was treated with oxalyl chloride (500 mg, 3.94 mmol, 0.35 mL) and stirred overnight. The solution was then concentrated to give 2-(1,2,4-triazol-1-yl)acetyl chloride, which was used without further purification assuming quantitative yield.

Step b: 2-(1,2,4-triazol-1-yl)acetyl chloride (291 mg, 2.00 mmol) in THF (2.5 mL) and MeCN (2.5 mL) was cooled to 0° C., and diazomethyl(trimethyl)silane (2 M, 1.2 mL) was added dropwise. The solution was stirred for 1 hour at 0° C., before conc. HCl (12 M, 500 μL) was added. The solution was stirred for 3 hours and allowed to warm to room temperature. The reaction was quenched by the addition of saturated aqueous NaHCO₃ (100 mL). The product was extracted with EtOAc (3×50 mL), dried (Na₂SO₄), and concentrated to give 1-chloro-3-(1,2,4-triazol-1-yl)propan-2-one, which was used without any further purification. ¹H NMR (500 MHz, CHLOROFORM-d) δ ppm 4.05 (s, 2H) 5.17 (s, 2H) 7.86 (s, 1H) 8.05 (s, 1H).

Step c: A vial was charged with N-(6-amino-4-ethoxy-3-pyridyl)-6-(difluoromethyl)pyridine-2-carboxamide (619 mg, 2.01 mmol), 1-chloro-3-(1,2,4-triazol-1-yl)propan-2-one (320 mg, 2.01 mmol) and EtOH (5 mL) followed by sodium bicarbonate (506 mg, 6.03 mmol). The vial was sealed at heated at 85° C. for 18 h. The mixture was then filtered, and the resulting solution was purified via TFA-modified reverse phase preparative HPLC to give 6-(difluoromethyl)-N-[7-ethoxy-2-(1,2,4-triazol-1-ylmethyl)imidazo[1,2-a]pyridin-6-yl]pyridine-2-carboxamide (9.3 mg, 17.7 μmol, 1% yield). LCMS (ESI) m/z 414.5 (M+H). ¹H NMR (500 MHz, METHANOL-d₄) δ ppm 1.62 (t, J=7.02 Hz, 3H) 4.30 (q, J=7.33 Hz, 2H) 5.53 (s, 2H) 6.72-6.97 (m, 2H) 7.79 (s, 1H) 7.95 (d, J=7.94 Hz, 1H) 8.03 (s, 1H) 8.25 (t, J=7.63 Hz, 1H) 8.37 (d, J=7.94 Hz, 1H) 8.57 (s, 1H) 9.51 (s, 1H).

### Example 83: N-(2-((1,4-dioxan-2-yl)methyl)-7-ethoxyimidazo[1,2-a]pyridin-6-yl)-6-(difluoromethyl)picolinamide

Example 83 was prepared in a similar fashion as Example 82: except 1,4-dioxane-2-carboxylic acid was used in place 2-(1,2,4-triazol-1-yl)acetic acid. LCMS (ESI) m/z 433.5 (M+H)⁺. H NMR (500 MHz, METHANOL-d₄) δ ppm 1.69 (t, J=7.02 Hz, 3H) 2.87-2.96 (m, 1H) 2.98 (d, J=3.66 Hz, 1H) 3.36-3.44 (m, 1H) 3.59-3.67 (m, 1H) 3.71-3.78 (m, 2H) 3.84-3.89 (m, 2H) 3.89-3.95 (m, 1H) 4.47 (q, J=7.32 Hz, 2H) 6.75-7.03 (m, 1H) 7.29 (s, 1H) 7.92 (s, 1H) 7.99 (d, J=7.94 Hz, 1H) 8.29 (t, J=7.63 Hz, 1H) 8.42 (d, J=7.33 Hz, 1H) 9.79 (s, 1H)

### Example 84: 6-(difluoromethyl)-N-(7-ethoxy-2-((1-methyl-1H-pyrazol-3-yl)methyl)imidazo[1,2-a]pyridin-6-yl)picolinamide

Example 84 was prepared in a similar fashion as Example 82: except 1-methyl-1H-pyrazole-3-carboxylic acid was used in place 2-(1,2,4-triazol-1-yl)acetic acid. LCMS (ESI) m/z 427.5 (M+H)⁺. ¹H NMR (500 MHz, METHANOL-d₄) δ ppm 1.68 (t, J=7.02 Hz, 4H) 3.90 (s, 3H) 4.09 (s, 2H) 4.47 (q, J=6.71 Hz, 2H) 6.75-7.01 (m, 1H) 7.26 (s, 1H) 7.49 (s, 1H) 7.63 (s, 1H) 7.84 (s, 1H) 7.99 (d, J=7.94 Hz, 1H) 8.29 (t, J=7.94 Hz, 1H) 8.42 (d, J=7.94 Hz, 1H) 9.78 (s, 1H).

### Intermediate 10: N-[2-(chloromethyl)-7-ethoxy-imidazo[1,2-a]pyridin-6-yl]-6-(difluoromethyl)pyridine-2-carboxamide

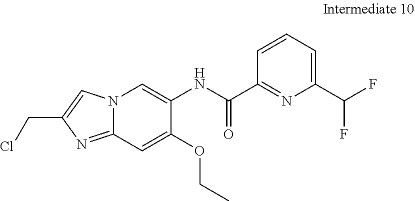 [SMILES: CCOc1cc2nc(CCl)cn2cc1NC(=O)c1cccc(C(F)F)n1]

A mixture of N-(6-amino-4-ethoxy-3-pyridyl)-6-(difluoromethyl)pyridine-2-carboxamide (499 mg, 1.62 mmol) and 1,3-dichloropropan-2-one (212 mg, 1.62 mmol) in MeCN (3 mL) and toluene (650 μL) was stirred at 100° C. overnight. Silica was then added, and the solution was then concentrated. purification via silica gel chromatography to give N-[2-(chloromethyl)-7-ethoxy-imidazo[1,2-a]pyridin-6-yl]-6-(difluoromethyl)pyridine-2-carboxamide (100.00 mg, 262.62 μmol, 16.21% yield). LCMS (ESI) m/z 381.3 (M+H)⁺. ¹H NMR (500 MHz, CHLOROFORM-d) δ ppm 1.62 (t, J=7.02 Hz, 3H) 4.17-4.33 (m, 2H) 4.73 (s, 2H) 6.52-6.83 (m, 1H) 6.92 (s, 1H) 7.50 (s, 1H) 7.85 (d, J=7.94 Hz, 1H) 8.12 (t, J=7.94 Hz, 1H) 8.38 (d, J=6.71 Hz, 1H) 9.46 (s, 1H).

### Example 85: 6-(difluoromethyl)-N-(7-ethoxy-2-((2-oxopyrrolidin-1-yl)methyl)imidazo[1,2-a]pyridin-6-yl)picolinamide

N-[2-(chloromethyl)-7-ethoxy-imidazo[1,2-a]pyridin-6-yl]-6-(difluoromethyl)pyridine-2-carboxamide (50 mg, 131 μmol), pyrrolidin-2-one (22 mg, 263 μmol, 20 μL), and K₂CO₃ (36 mg, 263 μmol) were dissolved in MeCN (0.5 mL) and the solution was heated to 50° C. overnight. Then NaHMDS (1 M in THF, 390 μL) was added, and the solution was stirred for 5 h at rt. The solution was quenched by the addition of a saturated aqueous solution of ammonium chloride (100 mL) and the resulting mixture was extracted with EtOAc (3×50 mL). The combined organic layers were dried (Na₂SO₄) and concentrated in vacuo. The resulting residue was purified via TFA-modified reverse phase preparative HPLC to give 6-(difluoromethyl)-N-[7-ethoxy-2-[(2-oxopyrrolidin-1-yl)methyl]imidazo[1,2-a]pyridin-6-yl]pyridine-2-carboxamide (2.5 mg, 4.6 μmol, 3.5% yield). LCMS (ESI) m/z 430.4 (M+H)⁺. ¹H NMR (500 MHz, METHANOL-d₄) δ ppm 1.69 (t, J=6.71 Hz, 3H) 2.09-2.19 (m, 2H) 2.49 (t, J=8.24 Hz, 2H) 3.53 (t, J=7.02 Hz, 2H) 4.49 (q, J=6.92 Hz, 2H) 4.71 (s, 2H) 6.73-7.09 (m, 1H) 7.31 (s, 1H) 7.99 (d, J=7.94 Hz, 1H) 8.07 (s, 1H) 8.30 (t, J=7.94 Hz, 1H) 8.43 (d, J=7.94 Hz, 1H) 9.82 (s, 1H).

### Example 86 and Example 87: 6-(difluoromethyl)-N-(7-ethoxy-2-((pyridin-2-yloxy)methyl)imidazo[1,2-a]pyridin-6-yl)picolinamide and 6-(difluoromethyl)-N-(7-ethoxy-2-((2-oxopyridin-1(2H)-yl)methyl)imidazo[1,2-a]pyridin-6-yl)picolinamide

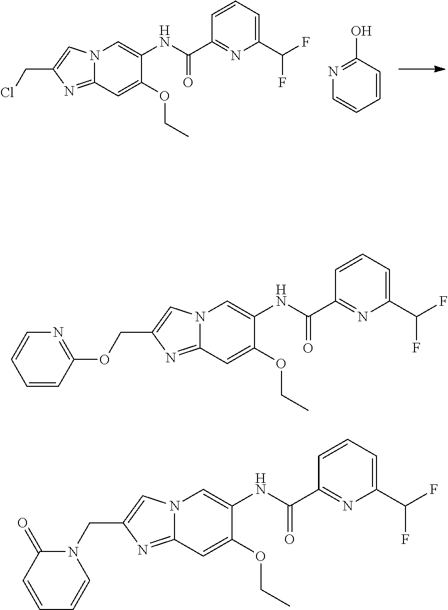

N-[2-(chloromethyl)-7-ethoxy-imidazo[1,2-a]pyridin-6-yl]-6-(difluoromethyl)pyridine-2-carboxamide (50 mg, 131 μmol), 1H-pyridin-2-one (25 mg, 262 μmol), and K₂CO₃ (36 mg, 263 μmol) were dissolved in MeCN (0.5 mL), and the solution was heated to 50° C. overnight. The solution was then filtered, and directly purified via TFA-modified reverse phase preparative HPLC to give Example 86: 6-(difluoromethyl)-N-[7-ethoxy-2-(2-pyridyloxymethyl)imidazo[1,2-a]pyridin-6-yl]pyridine-2-carboxamide (1.2 mg, 2.2 μmol, 2% yield) and Example 87: 6-(difluoromethyl)-N-[7-ethoxy-2-[(2-oxo-1-pyridyl)methyl]imidazo[1,2-a]pyridin-6-yl]pyridine-2-carboxamide (1.4 mg, 2.5 μmol, 2% yield).

### Example 86: 6-(difluoromethyl)-N-(7-ethoxy-2-((pyridin-2-yloxy)methyl)imidazo[1,2-a]pyridin-6-yl)picolinamide

LCMS (ESI) m/z 440.3 (M+H)⁺. ¹H NMR (500 MHz, METHANOL-d₄) δ ppm 1.67 (t, J=7.02 Hz, 3H) 4.46 (q, J=6.92 Hz, 2H) 5.59 (s, 2H) 6.73-6.98 (m, 2H) 7.01-7.07 (m, 1H) 7.31 (s, 1H) 7.73 (ddd, J=8.39, 6.87, 1.83 Hz, 1H) 7.97 (d, J=7.32 Hz, 1H) 8.18 (s, 1H) 8.21 (dd, J=4.88, 1.22 Hz, 1H) 8.27 (t, J=7.63 Hz, 1H) 8.41 (d, J=7.94 Hz, 1H) 9.82 (s, 1H).

### Example 87: 6-(difluoromethyl)-N-(7-ethoxy-2-((2-oxopyridin-1(2H)-yl)methyl)imidazo[1,2-a]pyridin-6-yl)picolinamide

LCMS (ESI) m/z 440.3 (M+H)⁺. ¹H NMR (500 MHz, METHANOL-d₄) δ ppm 1.62 (t, J=7.02 Hz, 3H) 4.30 (q, J=6.92 Hz, 2H) 5.26 (s, 2H) 6.42 (td, J=6.71, 1.22 Hz, 1H) 6.59 (d, J=9.16 Hz, 1H) 6.71-6.99 (m, 2H) 7.55 (ddd, J=8.85, 6.71, 2.14 Hz, 1H) 7.71 (s, 1H) 7.77 (dd, J=6.71, 1.83 Hz, 1H) 7.94 (d, J=7.32 Hz, 1H) 8.25 (t, J=7.63 Hz, 1H) 8.37 (d, J=7.94 Hz, 1H) 9.49 (s, 1H).

### Example 88: 1-(difluoromethyl)-N-(7-ethoxy-2-(3-methoxypropyl)imidazo[1,2-a]pyridin-6-yl)-1H-pyrazole-3-carboxamide

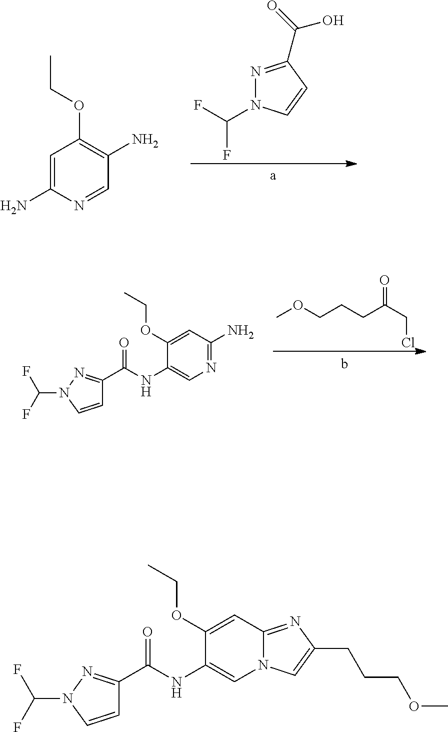

Step a: To a solution of 1-(difluoromethyl)-1H-pyrazole-3-carboxylic acid (87 mg, 539 μmol) in DMF (2 mL) was added HATU (226 mg, 593 μmol) and DIPEA (209 mg, 1.6 mmol, 283 μL). After stirring for 1-minute, Intermediate 3a, 4-ethoxypyridine-2,5-diamine (100 mg, 652 μmol) was added, and the mixture was stirred at 30° C. for 3 h. After this period, the solution was poured into water (25 mL), extracted with EtOAc (3×10 mL), dried over Na₂SO₄ and concentrated under reduced pressure to provide N-(6-amino-4-ethoxypyridin-3-yl)-1-(difluoromethyl)-1H-pyrazole-3-carboxamide (120 mg, 343 μmol, 63% yield) as a dark-red solid, which was used directly in the next step without additional purification. LCMS (ESI) m/z 298 (M+H)⁺.

Step b: N-(6-amino-4-ethoxypyridin-3-yl)-1-(difluoromethyl)-1H-pyrazole-3-carboxamide (90 mg, 302 μmol) and 1-chloro-5-methoxy-pentan-2-one (137 mg, 908 μmol) were dissolved together in EtOH (3 mL). To this solution, NaHCO₃ (76 mg, 908 μmol) was added, and the mixture was heated at reflux for 12 h. After cooling to rt, the solvent was evaporated. The residue was dissolved in EtOAc (50 mL), washed with H₂O (50 mL), dried over Na₂SO₄ and concentrated in vacuo. The residual red solid was purified by HPLC (Waters Xbridge Prep OBD C18 150×30 10 um, Gradient used 0-100% water in acetonitrile (0.04% NH₃H₂O+10 mM NH₄HCO₃ additive) to yield 1-(difluoromethyl)-N-(7-ethoxy-2-(3-methoxypropyl)imidazo[1,2-a]pyridin-6-yl)-1H-pyrazole-3-carboxamide (11 mg, 10% yield). LCMS (ESI) m/z 394.2 (M+H)⁺; 1H NMR (400 MHz, Methanol-d₄) δ 9.26 (s, 1H), 8.21 (d, J=2.7 Hz, 1H), 7.63 (t, J=59.4 Hz, 1H), 7.44 (s, 1H), 7.02 (d, J=2.7 Hz, 1H), 6.87 (s, 1H), 4.25 (q, J=7.0 Hz, 2H), 3.44 (t, J=6.4 Hz, 2H), 3.33 (s, 3H), 2.74 (t, J=7.7 Hz, 2H), 1.97 (m, 2H), 1.53 (t, J=7.0 Hz, 3H).

### Example 89: N-(2-((1R,6R,7R)-3-oxabicyclo[4.1.0]heptan-7-yl)-7-ethoxyimidazo[1,2-a]pyridin-6-yl)-6-(difluoromethyl)picolinamide

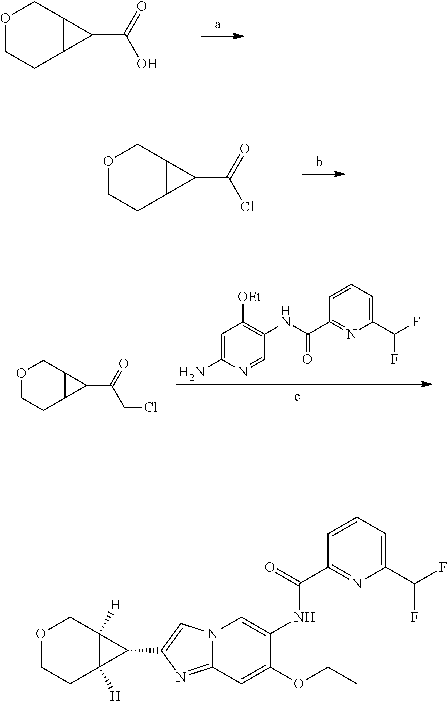 [SMILES: O=C(O)C1C2CCOCC21 ; O=C(Cl)C1C2CCOCC21 ; O=C(CCl)C1C2CCOCC21 ; CCOc1cc(N)ncc1NC(=O)c1cccc(C(F)F)n1 ; CCOc1cc2nc([C@H]3[C@@H]4COCC[C@@H]43)cn2cc1NC(=O)c1cccc(C(F)F)n1]

Step a: To a solution of 3-oxabicyclo[4.1.0]heptane-7-carboxylic acid (200 mg, 1.4 mmol) in DCM (5 mL) was added oxalyl chloride (357 mg, 2.8 mmol, 240 μL) at 0° C. under argon atmosphere. The mixture was stirred at 30° C. for 16 h. Then the mixture was concentrated in vacuo and residue was azeotroped with anhydrous DCM (3×5 mL) to give 3-oxabicyclo[4.1.0]heptane-7-carbonyl chloride as a yellow oil (220 mg, 1.37 mmol), which was used in the next step without any purification. GCMS (M:160).

Step b: To a solution of 3-oxabicyclo[4.1.0]heptane-7-carbonyl chloride (220 mg, 1.37 mmol) in THF (2.5 mL) and MeCN (2.5 mL) was added an Et₂O solution of diazomethyl(trimethyl)silane (2M, 685 μL) at 0° C. The mixture was stirred at 0° C. for 1 h at which time concentrated HCl (12 M, 353 μL) was added to the mixture at 0° C. The mixture was allowed to warm to rt and stirred at rt for 3 h. Then the mixture was diluted with EtOAc (20 mL) and basified with saturated aqueous NaHCO₃ to pH-8-9. The layers were separated, and the aqueous layer was extracted with EtOAc (3×20 mL). The combined organic layers were washed with brine (50 mL), dried over Na₂SO₄, filtered and concentrated in vacuo to give 1-(3-oxabicyclo[4.1.0]heptan-7-yl)-2-chloroethan-1-one (107 mg, 61% yield) as a dark brown viscous oil, which was used in the next step without further purification. LCMS (ESI) m/z 175 (M+H)⁺.

Step c: A 25-mL round-bottomed flask, equipped with a magnetic stirrer and a condenser, was charged with Intermediate 3c: N-(6-amino-4-ethoxypyridin-3-yl)-6-(difluoromethyl)picolinamide, Intermediate 3c: (145 mg, 471 μmol), 1-(3-oxabicyclo[4.1.0]heptan-7-yl)-2-chloroethan-1-one (107 mg, 613 μmol), NaHCO₃ (59.4 mg, 707 μmol) and EtOH (5 mL). The resulting mixture was heated at 80° C. overnight. Then, the solvent was evaporated in vacuo, H₂O (10 mL) was added, and the product was extracted with CHCl₃ (3×10 mL). The combined organic layers were dried over Na₂SO₄ and concentrated in vacuo to give a dark-violet viscous oil. The oil was purified by HPLC to provide N-(2-((1R,6R,7R)-3-oxabicyclo[4.1.0]heptan-7-yl)-7-ethoxyimidazo[1,2-a]pyridin-6-yl)-6-(difluoromethyl)picolinamide (34 mg, 17% yield) as a racemic mixture, designated stereochemistry is relative stereochemistry. LCMS (ESI) m/z 429 (M+H)⁺; 1H NMR (400 MHz, Chloroform-d) δ 10.50 (s, 1H), 9.37 (s, 1H), 8.36 (d, J=7.7 Hz, 1H), 8.08 (t, J=7.7 Hz, 1H), 7.81 (d, J=7.7 Hz, 1H), 7.23 (s, 1H), 6.83 (s, 1H), 6.64 (t, J=55.2 Hz, 1H), 4.21-4.11 (m, 2H), 4.05 (m, J=11.3 Hz, 1H), 3.97-3.88 (m, 1H), 3.61 (m, 1H), 3.31 (m, J=5.5 Hz, 1H), 1.98 (m, 2H), 1.66 (m, 2H), 1.57 (t, J=6.9 Hz, 3H), 1.22 (m, J=9.8 Hz, 1H).

### Example 90 and Example 91: 6-(difluoromethyl)-N-(7-ethoxy-2-(4-hydroxytetrahydro-2H-pyran-4-yl)imidazo[1,2-a]pyridin-6-yl)picolinamide and 6-(difluoromethyl)-N-(7-ethoxy-2-(4-ethoxytetrahydro-2H-pyran-4-yl)imidazo[1,2-a]pyridin-6-yl)picolinamide

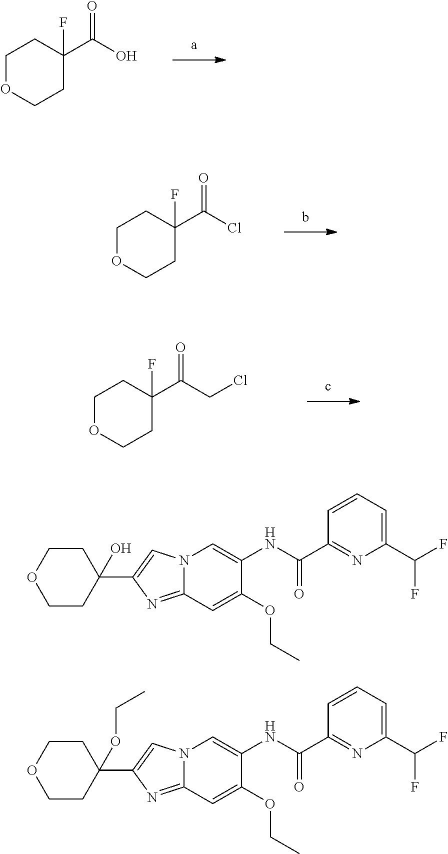 [SMILES: O=C(O)C1(F)CCOCC1 ; O=C(Cl)C1(F)CCOCC1 ; O=C(CCl)C1(F)CCOCC1 ; CCOc1cc2nc(C3(O)CCOCC3)cn2cc1NC(=O)c1cccc(C(F)F)n1 ; CCOc1cc2nc(C3(OCC)CCOCC3)cn2cc1NC(=O)c1cccc(C(F)F)n1]

Step a: 4-fluorotetrahydropyran-4-carboxylic acid (1.00 g, 6.75 mmol) in DCM (10 mL) was treated with oxalyl chloride (1.7 g, 14 mmol, 1.1 mL). The solution was stirred for 18 h, then concentrated to give 4-fluorotetrahydropyran-4-carbonyl chloride, which was used in the next step without further manipulation assuming quantitative yield. ¹H NMR (500 MHz, CHLOROFORM-d) δ ppm 1.89-2.07 (m, 2H) 2.15-2.34 (m, 2H) 3.72-3.86 (m, 2H) 3.90-4.03 (m, 2H).

Step b: 4-fluorotetrahydropyran-4-carbonyl chloride (1.12 g, 6.75 mmol) in MeCN (5 mL) and THF (5 mL) was cooled to 0° C., and diazomethyl(trimethyl)silane (2 M in ether, 4 mL) was added dropwise. The solution was maintained at 0° C. for 1 h, before conc. HCl (12 M, 1.7 mL) was added dropwise. The resulting solution was maintained at 0° C. for 3 h, and then quenched by the addition of a saturated aqueous solution of NaHCO₃ (100 mL). The layers were separated and the aqueous layer was extracted with EtOAc (3×50 mL), dried (Na₂SO₄), and concentrated to give 2-chloro-1-(4-fluorotetrahydropyran-4-yl)ethanone, which was used in the next step without further purification assuming quantitative yield. ¹H NMR (500 MHz, CHLOROFORM-d) δ ppm 1.54-1.68 (m, 2H) 1.89-2.14 (m, 2H) 3.58 (td, J=11.90, 2.44 Hz, 2H) 3.76 (ddd, J=11.45, 5.34, 1.53 Hz, 2H) 4.38 (d, J=3.05 Hz, 2H).

Step c: A vial was charged with N-(6-amino-4-ethoxy-3-pyridyl)-6-(difluoromethyl)pyridine-2-carboxamide (1.88 g, 6.09 mmol), 2-chloro-1-(4-fluorotetrahydropyran-4-yl)ethanone (1.10 g, 6.09 mmol), and EtOH (15 mL). To this mixture was added sodium bicarbonate (1.53 g, 18.27 mmol) and the vial was sealed and heated at 85° C. for 18 h. The reaction was filtered, and concentrated. The residue was purified by TFA-modified reverse phase HPLC to provide 6-(difluoromethyl)-N-[7-ethoxy-2-(4-hydroxytetrahydropyran-4-yl)imidazo[1,2-a]pyridin-6-yl]pyridine-2-carboxamide (Example 90, 9.6 mg, 17.6 μmol) and 6-(difluoromethyl)-N-[7-ethoxy-2-(4-ethoxytetrahydropyran-4-yl)imidazo[1,2-a]pyridin-6-yl]pyridine-2-carboxamide (Example 91, 304 mg, 530 μmol), data summarized below.

### Example 90: 6-(difluoromethyl)-N-(7-ethoxy-2-(4-hydroxytetrahydro-2H-pyran-4-yl)imidazo[1,2-a]pyridin-6-yl)picolinamide

LCMS (ESI) m/z 433.3 (M+H)⁺. ¹H NMR (500 MHz, METHANOL-d₄) δ ppm 1.57 (t, J=7.02 Hz, 3H) 1.83 (br dd, J=14.04, 1.83 Hz, 2H) 1.97-2.11 (m, 2H) 3.69-3.79 (m, 2H) 3.81-3.92 (m, 2H) 4.36 (q, J=6.71 Hz, 2H) 6.61-6.93 (m, 1H) 7.15 (s, 1H) 7.87 (d, J=7.94 Hz, 1H) 7.91 (s, 1H) 8.17 (t, J=7.63 Hz, 1H) 8.30 (d, J=7.94 Hz, 1H) 9.69 (s, 1H).

### Example 91: 6-(difluoromethyl)-N-(7-ethoxy-2-(4-ethoxytetrahydro-2H-pyran-4-yl)imidazo[1,2-a]pyridin-6-yl)picolinamide

LCMS (ESI) m/z 433.3 (M+H)⁺. ¹H NMR (500 MHz, METHANOL-d₄) δ ppm 1.57 (t, J=7.02 Hz, 3H) 1.83 (br dd, J=14.04, 1.83 Hz, 2H) 1.97-2.11 (m, 2H) 3.69-3.79 (m, 2H) 3.81-3.92 (m, 2H) 4.36 (q, J=6.71 Hz, 2H) 6.61-6.93 (m, 1H) 7.15 (s, 1H) 7.87 (d, J=7.94 Hz, 1H) 7.91 (s, 1H) 8.17 (t, J=7.63 Hz, 1H) 8.30 (d, J=7.94 Hz, 1H) 9.69 (s, 1H).

### Example 92: N-(2-cyclopropylimidazo[1,2-a]pyrimidin-6-yl)-6-(trifluoromethyl)picolinamide

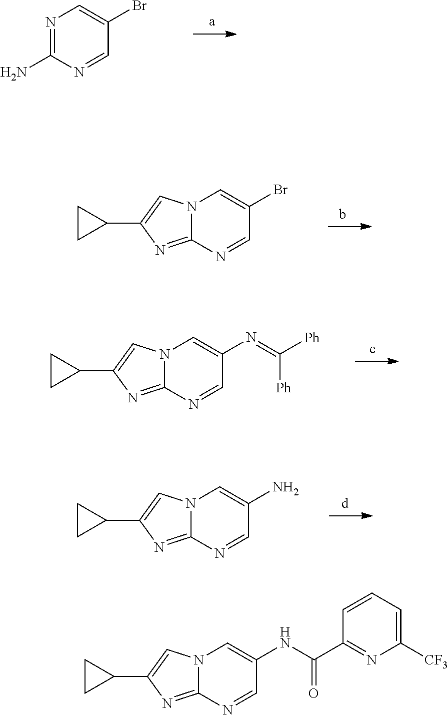 [SMILES: Nc1ncc(Br)cn1 ; Brc1cnc2nc(C3CC3)cn2c1 ; c1ccc(C(=Nc2cnc3nc(C4CC4)cn3c2)c2ccccc2)cc1 ; Nc1cnc2nc(C3CC3)cn2c1 ; O=C(Nc1cnc2nc(C3CC3)cn2c1)c1cccc(C(F)(F)F)n1]

Step a: A mixture of 5-bromopyrimidin-2-amine (650 mg, 3.74 mmol) and 2-bromo-1-cyclopropyl-ethanone (628 mg, 3.74 mmol, 375 μL) in MeCN (6 mL) and toluene (1.5 mL) was stirred at 100° C. for 16 h. Silica was then added, the solution was concentrated, and the mixture was purified by silica gel column chromatography to give 6-bromo-2-cyclopropyl-imidazo[1,2-a]pyrimidine (330 mg, 1.39 mmol, 37% yield). LCMS (ESI) m/z 240.1 (M+H)⁺.

Step b: A vial was charged with 6-bromo-2-cyclopropyl-imidazo[1,2-a]pyrimidine (330 mg, 1.39 mmol), [2-(2-aminophenyl)phenyl]-methylsulfonyloxy-palladium; ditert-butyl-[2-(2,4,6-triisopropylphenyl)phenyl]phosphane (55 mg, 69 μmol), K₃PO₄ (737 mg, 3.47 mmol). The vial was sealed with a septum cap and placed under an atmosphere of N₂. DME (3 mL) and benzophenone imine (300 mg, 1.67 mmol, 0.3 mL) were added via syringe. The mixture was heated to 45° C. for 18 h. Silica was added, and the solution was concentrated. Purification of the resulting mixture by silica gel column chromatography gave N-(2-cyclopropylimidazo[1,2-a]pyrimidin-6-yl)-1,1-diphenyl-methanimine (75 mg, 221 μmol, 16% yield). LCMS (ESI) m/z 339.2 (M+H)⁺.

Step c: N-(2-cyclopropylimidazo[1,2-a]pyrimidin-6-yl)-1,1-diphenyl-methanimine (75 mg, 221 μmol) in DCM (2 mL) and MeOH (2 mL) was treated with HCl (4 M in Dioxane, 0.25 mL) and stirred for 1 h. The reaction was then concentrated to give 2-cyclopropylimidazo[1,2-a]pyrimidin-6-amine hydrochloride which was used without further purification assuming quantitative yield. LCMS (ESI) m/z 175.2 (M+H)⁺.

Step d: 2-cyclopropylimidazo[1,2-a]pyrimidin-6-amine hydrochloride (26 mg, 126 μmol), 6-(trifluoromethyl)pyridine-2-carboxylic acid (28 mg, 149 μmol) in DMF (1.5 mL) were treated with DIPEA (77 mg, 597 μmol, 104 μL), followed by BOP (132 mg, 298 μmol). The reaction was stirred overnight at room temperature. The material was then directly purified via reverse phase HPLC to give N-(2-cyclopropylimidazo[1,2-a]pyrimidin-6-yl)-6-(trifluoromethyl)picolinamide (600 ug, 1.73 μmol, 1.2% yield). LCMS (ESI) m/z 348.2 (M+H)⁺. ¹H NMR (500 MHz, METHANOL-d₄) δ ppm 1.04-1.10 (m, 2H) 1.27 (br dd, J=8.55, 2.44 Hz, 2H) 2.18-2.29 (m, 1H) 7.89 (s, 1H) 8.06 (d, J=4.88 Hz, 1H) 8.22 (d, J=7.94 Hz, 1H) 8.41 (t, J=7.94 Hz, 1H) 8.58 (d, J=7.94 Hz, 1H) 8.88 (d, J=5.49 Hz, 1H).

### Example 93: N-(7-ethoxy-2-((tetrahydrofuran-3-yl)methyl)imidazo[1,2-a]pyrimidin-6-yl)-6-(trifluoromethyl)picolinamide

Example 93 was prepared in a similar fashion as Example 74, except 1-chloro-3-(tetrahydrofuran-3-yl)propan-2-one used in place of bromobutan-2-one, and 6-(trifluoromethyl)picolinic acid used in place of 6-(difluoromethyl)pyridine-2-carboxamide. LCMS (ESI) m/z 436.3 (M+H)⁺. ¹H NMR (500 MHz, METHANOL-d₄) δ ppm 1.64 (t, J=7.02 Hz, 3H) 1.70-1.80 (m, 1H) 2.11-2.25 (m, 1H) 2.62-2.77 (m, 1H) 2.93 (dd, J=7.63, 2.75 Hz, 2H) 3.56 (dd, J=8.55, 5.49 Hz, 1H) 3.82 (q, J=7.73 Hz, 1H) 3.88-3.99 (m, 2H) 4.78 (q, J=7.12 Hz, 2H) 7.84 (s, 1H) 8.16 (d, J=7.94 Hz, 1H) 8.39 (t, J=7.94 Hz, 1H) 8.53 (d, J=7.33 Hz, 1H) 9.91 (s, 1H).

### Example 94: (S)—N-(7-ethoxy-2-((tetrahydrofuran-3-yl)methyl)imidazo[1,2-a]pyrimidin-6-yl)-6-(trifluoromethyl)picolinamide

Example 94 was obtained as Peak 1 from chiral HPLC separation of Example 93: Column: CHIRALPAK IG 30×250 mm, 5 um Method: 45% MeOH w/0.1% DEA in CO2 (flow rate: 100 mL/min, ABPR 120 bar, MBPR 40 psi, column temp 40 deg C.)) to provide (S)—N-(7-ethoxy-2-((tetrahydrofuran-3-yl)methyl)imidazo[1,2-a]pyrimidin-6-yl)-6-(trifluoromethyl)picolinamide (>99% ee). ¹H NMR (500 MHz, METHANOL-d₄) δ ppm 1.55 (t, J=7.33 Hz, 3H) 1.67-1.75 (m, 1H) 2.04-2.15 (m, 1H) 2.71 (dd, J=14.04, 6.71 Hz, 1H) 2.75-2.78 (m, 2H) 3.51 (dd, J=8.55, 6.10 Hz, 1H) 3.73-3.82 (m, 1H) 3.85-3.94 (m, 2H) 4.63 (q, J=6.92 Hz, 2H) 7.45 (s, 1H) 8.09 (d, J=7.94 Hz, 1H) 8.33 (t, J=7.63 Hz, 1H) 8.48 (d, J=7.94 Hz, 1H) 9.62 (s, 1H).

### Example 95: (R)—N-(7-ethoxy-2-((tetrahydrofuran-3-yl)methyl)imidazo[1,2-a]pyrimidin-6-yl)-6-(trifluoromethyl)picolinamide

Example 95 was obtained as Peak 2 from chiral HPLC separation of Example 93, Column: CHIRALPAK IG 30×250 mm, 5 um Method: 45% MeOH w/0.1% DEA in CO2 (flow rate: 100 mL/min, ABPR 120 bar, MBPR 40 psi, column temp 40 deg C.)) to provide (R)—N-(7-ethoxy-2-((tetrahydrofuran-3-yl)methyl)imidazo[1,2-a]pyrimidin-6-yl)-6-(trifluoromethyl)picolinamide (95% ee). ¹H NMR (500 MHz, METHANOL-d₄) δ ppm 1.55 (t, J=7.02 Hz, 3H) 1.70 (dd, J=12.21, 6.71 Hz, 1H) 2.09 (dd, J=12.51, 5.19 Hz, 1H) 2.65-2.73 (m, 1H) 2.74-2.79 (m, 2H) 3.51 (dd, J=8.55, 6.10 Hz, 1H) 3.77 (q, J=7.73 Hz, 1H) 3.84-3.93 (m, 2H) 4.62 (q, J=7.12 Hz, 2H) 7.44 (s, 1H) 8.09 (d, J=7.94 Hz, 1H) 8.33 (t, J=7.94 Hz, 1H) 8.47 (d, J=7.94 Hz, 1H) 9.61 (s, 1H).

### Examples 96 and 97: N-(7-ethoxy-2-((1r,3r)-3-hydroxycyclobutyl)imidazo[1,2-a]pyridin-6-yl)-6-(trifluoromethyl)picolinamide and N-(7-ethoxy-2-((1s,3s)-3-hydroxycyclobutyl)imidazo[1,2-a]pyridin-6-yl)-6-(trifluoromethyl)picolinamide

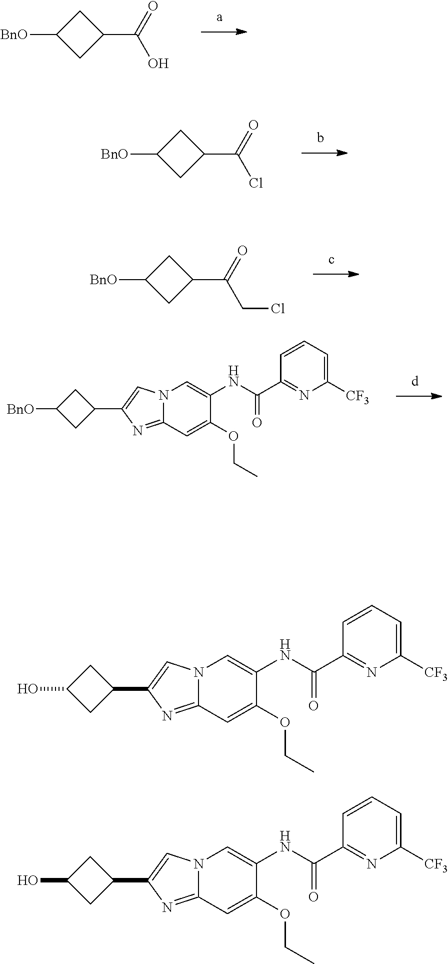

Step a: 3-benzyloxycyclobutanecarboxylic acid (mixture of cis/trans isomers, 1.0 g, 4.9 mmol, 280 μL) in DCM (7 mL) was treated with oxalyl chloride (1.2 g, 9.7 mmol, 0.82 mL). The solution was maintained at rt for 16 h. The solution was then concentrated to give 3-benzyloxycyclobutanecarbonyl chloride as a mixture of cis/trans isomers, which was used directly without further purification assuming quantitative yield.

Step b: A solution of 3-benzyloxycyclobutanecarbonyl chloride (mixture of cis/trans isomers, 1.1 g, 4.9 mmol) in MeCN (5 mL) and THF (5 mL) was cooled to 0° C., and diazomethyl(trimethyl)silane (2 M, 3.0 ml) was added. The solution was maintained at 0° C. for 1 h, and then conc. HCl (12 M, 1.25 ml) was added dropwise. The solution was stirred at 0° C. for 2 h, and then quenched by the addition of a saturated solution of NaHCO₃ (100 mL). The layers were separated and the organic layer was extracted with EtOAc (3×50 mL), dried (Na₂SO₄), and concentrated to give 1-(3-benzyloxycyclobutyl)-2-chloro-ethanone as a mixture of cis/trans isomers, which was used without further purification assuming quantitative yield.

Step c: A vial was charged with N-(6-amino-4-ethoxy-3-pyridyl)-6-(difluoromethyl)pyridine-2-carboxamide (1.42 g, 4.61 mmol), 1-(3-benzyloxycyclobutyl)-2-chloro-ethanone (1.1 g, 4.6 mmol) and EtOH (12 mL). Sodium bicarbonate (1.16 g, 13.8 mmol) was added and the vial was sealed at heated at 85° C. for 18 h. Silica gel was then added, and the mixture was concentrated.

Purification of the mixture by silica gel chromatography gave N-[2-(3-benzyloxycyclobutyl)-7-ethoxy-imidazo[1,2-a]pyridin-6-yl]-6-(difluoromethyl)pyridine-2-carboxamide as a mixture of cis/trans isomers (670 mg, 1.36 mmol, 30% yield). LCMS (ESI) m/z 493.3 (M+H)⁺. ¹H NMR (500 MHz, CHLOROFORM-d) δ ppm 0.80 (t, J=7.02 Hz, 3H) 1.52 (td, J=7.02, 2.44 Hz, 3H) 2.11-2.28 (m, 1H) 2.43-2.52 (m, 2H) 2.58-2.69 (m, 1H) 2.80 (s, 1H) 2.88 (s, 1H) 2.97-3.10 (m, 1H) 3.51-3.69 (m, 1H) 3.94-4.09 (m, 2H) 4.09-4.22 (m, 2H) 4.36 (br d, J=6.71 Hz, 1H) 4.40 (s, 2H) 6.41-6.72 (m, 1H) 6.77-6.90 (m, 1H) 7.02-7.39 (m, 7H) 7.75 (d, J=7.33 Hz, 1H) 8.02 (t, J=7.94 Hz, 1H) 8.30 (d, J=7.94 Hz, 1H) 9.34 (s, 1H) 10.45 (s, 1H).

Step d: N-[2-(3-benzyloxycyclobutyl)-7-ethoxy-imidazo[1,2-a]pyridin-6-yl]-6-(difluoromethyl)pyridine-2-carboxamide (670 mg, 1.36 mmol) and palladium on carbon (145 mg, 1.36 mmol, 10% w/w) were dissolved in a blend of 3:1 EtOAc:EtOH (50 mL), and placed under an atmosphere of H₂ for 24 h. The solution was then filtered through Celite, concentrated, and purified via SFC to give N-(7-ethoxy-2-((1r,3r)-3-hydroxycyclobutyl)imidazo[1,2-a]pyridin-6-yl)-6-(trifluoromethyl)picolinamide and N-(7-ethoxy-2-((1s,3s)-3-hydroxycyclobutyl)imidazo[1,2-a]pyridin-6-yl)-6-(trifluoromethyl)picolinamide. Data summarized below.

### Purification Details:

CHIRALPAK IB 30×250 mm, 5 μm
Method: 25% MeOH w/0.1% DEA in CO₂ (flow rate: 100 mL/min, ABPR 120 bar, MBPR 40 psi, column temp 40° C.)
Collection mode was UV collection monitoring at 282 nm.

### Example 96: N-(7-ethoxy-2-((1r,3r)-3-hydroxycyclobutyl)imidazo[1,2-a]pyridin-6-yl)-6-(trifluoromethyl)picolinamide

LCMS (ESI) m/z 403.3 (M+H)⁺. ¹H NMR (400 MHz, METHANOL-d₄) δ ppm 1.60 (t, J=7.03 Hz, 3H) 2.02-2.20 (m, 2H) 2.63-2.77 (m, 2H) 3.01 (m, 1H) 4.14-4.35 (m, 1H) 4.28 (q, J=6.86 Hz, 2H) 6.67-7.01 (m, 2H) 7.49 (s, 1H) 7.92 (d, J=7.53 Hz, 1H) 8.24 (t, J=7.78 Hz, 1H) 8.33-8.41 (m, 1H) 9.44 (s, 1H).

### Example 97: N-(7-ethoxy-2-((1s,3s)-3-hydroxycyclobutyl)imidazo[1,2-a]pyridin-6-yl)-6-(trifluoromethyl)picolinamide

LCMS (ESI) m/z 403.3 (M+H)⁺. ¹H NMR (400 MHz, METHANOL-d₄) δ ppm 1.60 (t, J=7.03 Hz, 3H) 2.32-2.46 (m, 2H) 2.48-2.61 (m, 2H) 3.46-3.65 (m, 1H) 4.28 (q, J=6.53 Hz, 2H) 4.51 (t, J=7.28 Hz, 1H) 6.67-7.01 (m, 2H) 7.55 (s, 1H) 7.93 (d, J=7.28 Hz, 1H) 8.24 (t, J=7.91 Hz, 1H) 8.36 (d, J=7.53 Hz, 1H) 9.44 (s, 1H).

### Intermediate 12: Ethyl 6-(6-(difluoromethyl)picolinamido)-7-ethoxyimidazo[1,2-a]pyridine-2-carboxylate

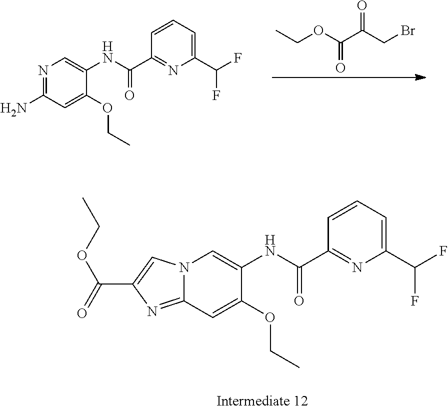

Ethyl 7-ethoxy-6-(6-(trifluoromethyl)picolinamido)imidazo[1,2-a]pyridine-2-carboxylate was prepared in a similar manner to that described for Intermediate 1, substituting ethyl 3-bromo-2-oxopropanoate in place of 1-bromobutan-2-one. LCMS (ESI) m/z 423.2 (M+H)⁺.

### Intermediate 13: methyl 1-(6-(6-(difluoromethyl)picolinamido)-7-ethoxyimidazo[1,2-a]pyridin-2-yl)cyclopropane-1-carboxylate

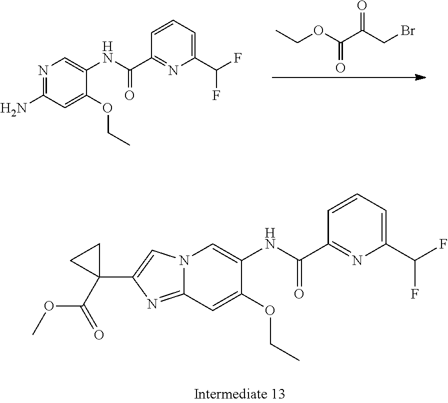

Methyl-2-(1-(6-(6-(difluoromethyl)picolinamido)-7-ethoxyimidazo[1,2-a]pyridin-2-yl)cyclopropyl)acetate was prepared in a similar manner to that described for Intermediate 1, substituting methyl 1-(2-bromoacetyl)cyclopropane-1-carboxylate in place of 1-bromobutan-2-one. LCMS (ESI) m/z 431.3 (M+H)⁺.

### Example 98: 6-(difluoromethyl)-N-(7-ethoxy-2-(1-(hydroxymethyl)cyclopropyl)imidazo[1,2-a]pyridin-6-yl)picolinamide

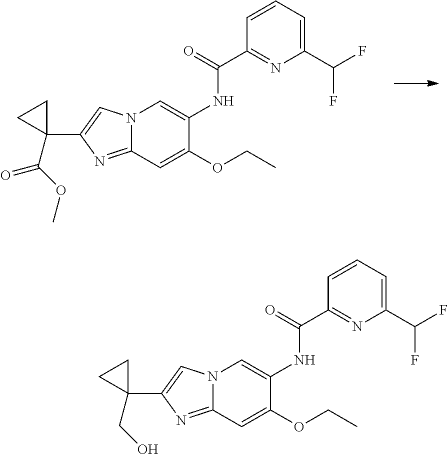 [SMILES: CCOc1cc2nc(C3(C(=O)OC)CC3)cn2cc1NC(=O)c1cccc(C(F)F)n1 ; CCOc1cc2nc(C3(CO)CC3)cn2cc1NC(=O)c1cccc(C(F)F)n1]

6-(difluoromethyl)-N-(7-ethoxy-2-(1-(hydroxymethyl)cyclopropyl)imidazo[1,2-a]pyridin-6-yl)picolinamide was prepared in a similar manner to that described for Example 17. LCMS (ESI) m/z 403.2 (M+H)+, ¹H NMR (400 MHz, METHANOL-d₄) δ ppm 1.11 (dt, J=4.27, 1.63 Hz, 4H) 1.66 (t, J=7.03 Hz, 3H) 3.75 (s, 2H) 4.44 (q, J=6.86 Hz, 2H) 6.70-7.02 (m, 1H) 7.23 (s, 1H) 7.89 (s, 1H) 7.96 (d, J=7.78 Hz, 1H) 8.26 (t, J=7.78 Hz, 1H) 8.39 (d, J=7.78 Hz, 1H) 9.73 (s, 1H).

### Intermediate 14: methyl 3-(6-(6-(difluoromethyl)picolinamido)-7-ethoxyimidazo[1,2-a]pyridin-2-yl)-2-fluoropropanoate

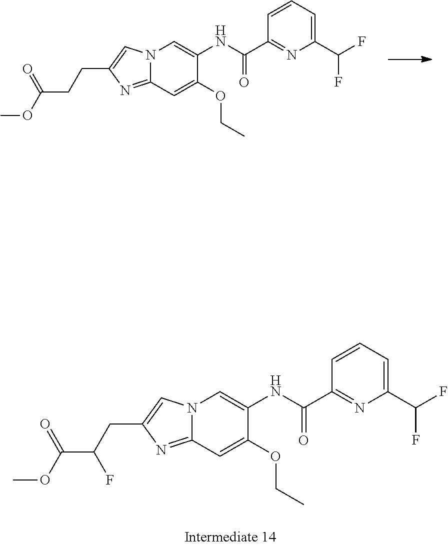 [SMILES: CCOc1cc2nc(CCC(=O)OC)cn2cc1NC(=O)c1cccc(C(F)F)n1 ; CCOc1cc2nc(CC(F)C(=O)OC)cn2cc1NC(=O)c1cccc(C(F)F)n1]

A THF solution of [bis(trimethylsilyl)amino]lithium (1 M, 1.7 mL) was added to a solution of methyl 3-(6-(6-(difluoromethyl)picolinamido)-7-ethoxyimidazo[1,2-a]pyridin-2-yl)propanoate from Example 23, Step a (208 mg, 497 μmol) in THF (2.5 mL) at −78° C. The resulting solution was allowed to warm to 0° C. and then maintained at 0° C. for 30 min. After cooling to −78° C., N-(benzenesulfonyl)-N-fluoro-benzenesulfonamide (470 mg, 1.49 mmol) was added as THF solution (in 1.5 mL) and the mixture was allowed to warm to rt overnight. The reaction mixture was quenched by the addition of NaHCO₃, the layers were separated and the aqueous layer was extracted with EtOAc (3×20 mL), dried over MgSO₄, filtered, concentrated, and purified via silica gel chromatography using a gradient of 0-100% of a blend of 3:1 EtOAc:EtOH in heptanes to obtain methyl 3-(6-(6-(difluoromethyl)picolinamido)-7-ethoxyimidazo[1,2-a]pyridin-2-yl)-2-fluoropropanoate (30 mg, 14% yield); LCMS (ESI) m/z 437.2 (M+H)⁺.

### Example 99: 6-(difluoromethyl)-N-(7-ethoxy-2-(2-fluoro-3-hydroxypropyl)imidazo[1,2-a]pyridin-6-yl)picolinamide

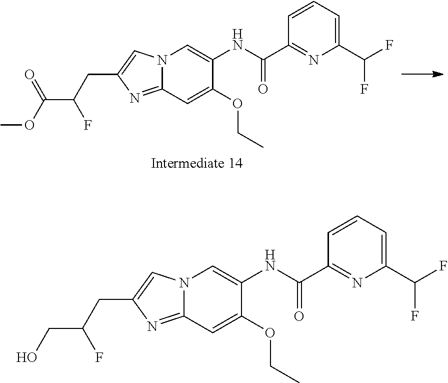 [SMILES: CCOc1cc2nc(CC(F)C(=O)OC)cn2cc1NC(=O)c1cccc(C(F)F)n1 ; CCOc1cc2nc(CC(F)CO)cn2cc1NC(=O)c1cccc(C(F)F)n1]

6-(difluoromethyl)-N-(7-ethoxy-2-(2-fluoro-3-hydroxypropyl)imidazo[1,2-a]pyridin-6-yl)picolinamide was prepared in a similar manner to that described for Example 17. LCMS (ESI) m/z 409.2 (M+H)⁺, ¹H NMR (400 MHz, METHANOL-d₄) δ ppm 1.65-1.71 (m, 1H) 1.67-1.69 (m, 1H) 1.71 (s, 1H) 3.19-3.32 (m, 1H) 3.19-3.30 (m, 1H) 3.34-3.39 (m, 1H) 3.69-3.90 (m, 2H) 4.45-4.52 (m, 2H) 6.74-7.04 (m, 1H) 7.29-7.35 (m, 1H) 7.95-8.02 (m, 2H) 8.26-8.33 (m, 1H) 8.40-8.45 (m, 1H) 9.80 (s, 1H).

### Example 100: 6-(difluoromethyl)-N-(7-ethoxy-2-(2-hydroxypropan-2-yl)imidazo[1,2-a]pyridin-6-yl)picolinamide

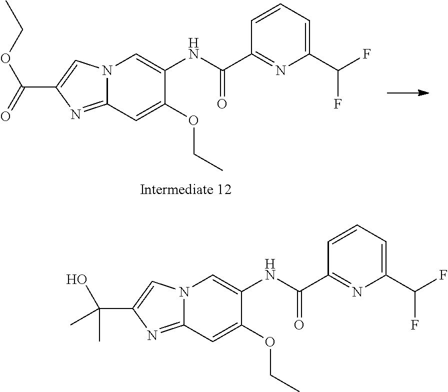 [SMILES: CCOC(=O)c1cn2cc(NC(=O)c3cccc(C(F)F)n3)c(OCC)cc2n1 ; CCOc1cc2nc(C(C)(C)O)cn2cc1NC(=O)c1cccc(C(F)F)n1]

6-(difluoromethyl)-N-(7-ethoxy-2-(2-hydroxypropan-2-yl)imidazo[1,2-a]pyridin-6-yl)picolinamide was prepared in a similar manner to that described for Example 17: starting from ethyl 6-(6-(difluoromethyl)picolinamido)-7-ethoxyimidazo[1,2-a]pyridine-2-carboxylate. LCMS (ESI) m/z 391.3 (M+H)⁺, ¹H NMR (400 MHz, METHANOL-d₄) δ ppm 1.64-1.70 (m, 9H) 4.42-4.49 (m, 2H) 6.71-7.02 (m, 1H) 7.24 (s, 1H) 7.92-7.98 (m, 2H) 8.26 (t, J=7.78 Hz, 1H) 8.39 (dd, J=7.78, 0.75 Hz, 1H) 9.76 (s, 1H).

### Example 101: N-(2-cyclopropyl-7-(2-methoxyethoxy)imidazo[1,2-a]pyridin-6-yl)-6-(difluoromethyl)picolinamide

N-(2-cyclopropyl-7-(2-methoxyethoxy)imidazo[1,2-a]pyridin-6-yl)-6-(difluoromethyl)picolinamide was prepared in a similar manner to that described for Example 5: substituting 2-methoxyethan-1-ol in place of cyclobutylmethanol. LCMS (ESI) m/z 403.2 (M+H)⁺, ¹H NMR (400 MHz, METHANOL-d₄) δ ppm 0.90-0.96 (m, 2H) 1.14-1.20 (m, 2H) 2.04-2.13 (m, 1H) 3.52 (s, 3H) 3.92-4.00 (m, 2H) 4.49-4.55 (m, 2H) 6.71-7.02 (m, 1H) 7.30 (s, 1H) 7.74-7.79 (m, 1H) 7.96 (d, J=7.78 Hz, 1H) 8.26 (t, J=7.78 Hz, 1H) 8.38 (d, J=7.53 Hz, 1H) 9.71 (d, J=2.76 Hz, 1H).

### Example 102: N-(2-cyclopropyl-7-(2-morpholinoethoxy)imidazo[1,2-a]pyridin-6-yl)-6-(difluoromethyl)picolinamide

N-(2-cyclopropyl-7-(2-morpholinoethoxy)imidazo[1,2-a]pyridin-6-yl)-6-(difluoromethyl)picolinamide was prepared in a similar manner to that described for Example 5: substituting 2-morpholinoethan-1-ol in place of cyclobutylmethanol. LCMS (ESI) m/z 458.3 (M+H)⁺, ¹H NMR (400 MHz, METHANOL-d₄) δ ppm 0.93-0.98 (m, 2H) 1.16-1.23 (m, 2H) 2.09-2.17 (m, 1H) 3.44-3.64 (m, 3H) 3.87-4.02 (m, 6H) 4.83-4.87 (m, 2H) 6.87-7.16 (m, 1H) 7.40 (s, 1H) 7.83 (s, 1H) 7.94-8.00 (m, 1H) 8.27 (t, J=7.78 Hz, 1H) 8.42 (dd, J=7.78, 0.75 Hz, 1H) 9.71 (s, 1H).

### Example 103: N-(8-(benzyloxy)-2-(tetrahydro-2H-pyran-4-yl)imidazo[1,2-a]pyridin-6-yl)-6-(trifluoromethyl)picolinamide

N-(8-(benzyloxy)-2-(tetrahydro-2H-pyran-4-yl)imidazo[1,2-a]pyridin-6-yl)-6-(trifluoromethyl)picolinamide was prepared in a similar manner to that described for Example 28: substituting phenylmethanol in place of ethanol and 2-bromo-1-(tetrahydro-2H-pyran-4-yl)ethan-1-one in place of 2-bromo-1-cyclopropylethan-1-one. LCMS (ESI) m/z 497.2 (M+H)⁺, ¹H NMR (400 MHz, METHANOL-d₄) δ ppm 1.73-1.87 (m, 2H) 1.96-2.03 (m, 2H) 2.93-3.01 (m, 1H) 3.58 (td, J=11.67, 2.01 Hz, 2H) 3.98-4.06 (m, 2H) 5.31 (s, 2H) 7.11 (d, J=1.51 Hz, 1H) 7.31-7.43 (m, 3H) 7.55-7.59 (m, 2H) 7.61 (s, 1H) 8.04 (dd, J=7.78, 0.75 Hz, 1H) 8.28 (t, J=7.91 Hz, 1H) 8.44 (d, J=7.78 Hz, 1H) 8.89 (d, J=1.51 Hz, 1H).

### Example 104: N-(8-hydroxy-2-(tetrahydro-2H-pyran-4-yl)imidazo[1,2-a]pyridin-6-yl)-6-(trifluoromethyl)picolinamide

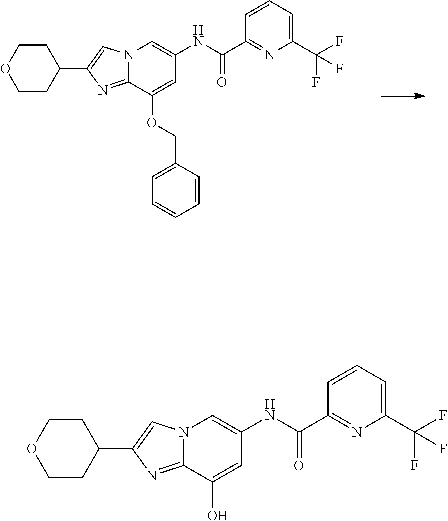 [SMILES: O=C(Nc1cc(OCc2ccccc2)c2nc(C3CCOCC3)cn2c1)c1cccc(C(F)(F)F)n1 ; O=C(Nc1cc(O)c2nc(C3CCOCC3)cn2c1)c1cccc(C(F)(F)F)n1]

A blend of EtOAc:EtOH 3:1 (3 mL) was added to a mixture of Example 103: N-(8-benzyloxy-2-tetrahydropyran-4-yl-imidazo[1,2-a]pyridin-6-yl)-6-(trifluoromethyl)picolinamide (265 mg, 533 μmol) and palladium on carbon 10 wt % (11.4 mg, 107 μmol) at rt and stirred under a balloon of H₂ gas for 18 h. The mixture was filtered through Celite (eluting with DCM), concentrated and purified via silica column chromatography using a gradient of 0-70% of a blend of 3:1 EtOAc:EtOH in heptanes to obtain N-(8-hydroxy-2-(tetrahydro-2H-pyran-4-yl)imidazo[1,2-a]pyridin-6-yl)-6-(trifluoromethyl)picolinamide (69 mg, 170 μmol, 32% yield), LCMS (ESI) m/z 407.2 (M+H)+, ¹H NMR (400 MHz, METHANOL-d₄) δ ppm 1.77-1.89 (m, 4H) 2.02 (br d, J=13.30 Hz, 4H) 2.97-3.07 (m, 2H) 3.60 (br t, J=11.80 Hz, 4H) 4.01-4.08 (m, 4H) 6.54 (d, J=7.53 Hz, 1H) 6.71 (t, J=7.15 Hz, 1H) 6.83 (d, J=1.76 Hz, 1H) 7.57-7.66 (m, 2H) 7.83 (d, J=6.53 Hz, 1H) 8.06 (d, J=8.03 Hz, 1H) 8.29 (t, J=7.91 Hz, 1H) 8.45 (d, J=7.78 Hz, 1H) 8.76 (d, J=1.76 Hz, 1H).

### Example 105: N-(8-methoxy-2-tetrahydropyran-4-yl-imidazo[1,2-a]pyridin-6-yl)-6-(trifluoromethyl)picolinamide

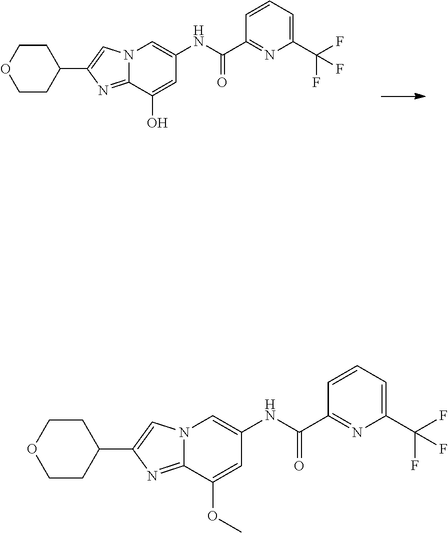 [SMILES: O=C(Nc1cc(O)c2nc(C3CCOCC3)cn2c1)c1cccc(C(F)(F)F)n1 ; COc1cc(NC(=O)c2cccc(C(F)(F)F)n2)cn2cc(C3CCOCC3)nc12]

A mixture of Example 104: N-(8-hydroxy-2-tetrahydropyran-4-yl-imidazo[1,2-a]pyridin-6-yl)-6-(trifluoromethyl)picolinamide (45 mg, 110 μmol), dipotassium carbonate (31 mg, 221 μmol) and iodomethane (16 mg, 110 μmol) was stirred at 60° C. for 2 h. The mixture was quenched by the addition of brine (10 mL), extracted with EtOAc (2×10 mL), washed with brine (10 mL), dried over MgSO₄, concentrated and purified by TFA-modified mass-directed HPLC on a reverse phase column (5-95% gradient of water to acetonitrile) to obtain N-(8-methoxy-2-tetrahydropyran-4-yl-imidazo[1,2-a]pyridin-6-yl)-6-(trifluoromethyl)picolinamide (14.1 mg, 33.5 μmol, 30% yield); LCMS (ESI) m/z 421.3 (M+H)⁺, ¹H NMR (400 MHz, METHANOL-d₄) δ ppm 1.87 (qd, J=12.30, 4.27 Hz, 2H) 2.04 (br dd, J=12.80, 1.76 Hz, 2H) 3.12-3.21 (m, 1H) 3.60 (td, J=11.73, 1.88 Hz, 2H) 4.07 (dd, J=11.04, 3.51 Hz, 2H) 4.17 (s, 3H) 7.71 (br s, 1H) 8.04 (br s, 1H) 8.10 (dd, J=7.78, 1.00 Hz, 1H) 8.32 (t, J=7.91 Hz, 1H) 8.49 (d, J=7.78 Hz, 1H) 9.30 (br s, 1H).

### Intermediate 15: 8-bromo-6-nitro-2-(tetrahydro-2H-pyran-4-yl)imidazo[1,2-a]pyridine

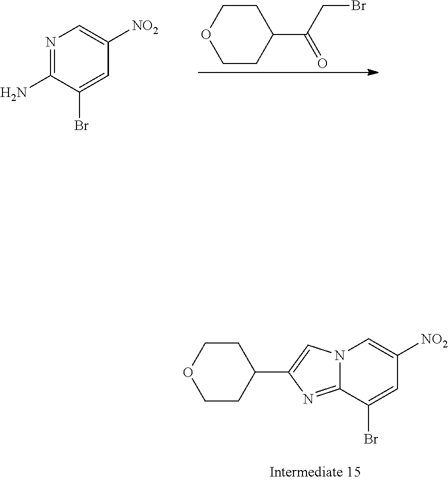 [SMILES: Nc1ncc([N+](=O)[O-])cc1Br ; O=C(CBr)C1CCOCC1 ; O=[N+]([O-])c1cc(Br)c2nc(C3CCOCC3)cn2c1]

A mixture of 3-bromo-5-nitro-pyridin-2-amine (1.0 g, 4.59 mmol), 2-bromo-1-tetrahydropyran-4-yl-ethanone (950 mg, 4.59 mmol), and sodium hydrogen carbonate (1.16 g, 13.8 mmol) in MeCN (9.2 mL) and Toluene (2.3 mL) was stirred at 100° C. for 16 h. After cooling to rt, MeOH and silica were added, concentrated, purified by silica column chromatography (0-80% gradient of a 3:1 EtOAc:EtOH blend in heptanes) to obtain 1.05 g of an inseparable mixture of 3-bromo-5-nitro-pyridin-2-amine and 8-bromo-6-nitro-2-tetrahydropyran-4-yl-imidazo[1,2-a]pyridine (1:2 ratio) which was used without further purification in the next reaction. LCMS (ESI) m/z 328.0

### Intermediate 16: 8-bromo-2-(tetrahydro-2H-pyran-4-yl)imidazo[1,2-a]pyridin-6-amine

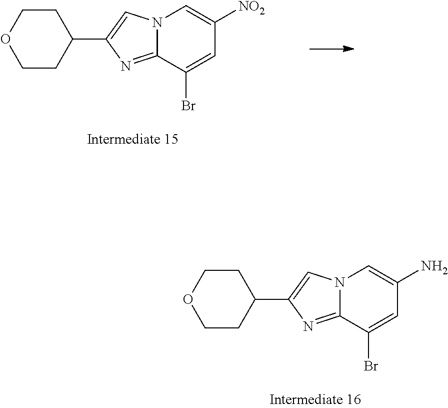 [SMILES: O=[N+]([O-])c1cc(Br)c2nc(C3CCOCC3)cn2c1 ; Nc1cc(Br)c2nc(C3CCOCC3)cn2c1]

A mixture of 8-bromo-6-nitro-2-tetrahydropyran-4-yl-imidazo[1,2-a]pyridine (950 mg, 2.91 mmol) and dichlorotin; dihydrate (1.97 g, 8.73 mmol) in EtOH (5 mL) was stirred at 70° C. for 1 h. After cooling to rt, the mixture was quenched by the addition of ice water (10 mL), diluted with EtOAc (50 mL) and saturated aqueous NaHCO₃ (50 mL). The layers were separated and the aqueous layer was extracted with EtOAc (3×20 mL), washed with brine (20 mL), dried over MgSO₄, filtered, and concentrated to obtain 8-bromo-2-tetrahydropyran-4-yl-imidazo[1,2-a]pyridin-6-amine which was used without further purification in next reaction. LCMS (ESI) m/z 204.1

### Example 106: N-(8-bromo-2-(tetrahydro-2H-pyran-4-yl)imidazo[1,2-a]pyridin-6-yl)-6-(trifluoromethyl)picolinamide

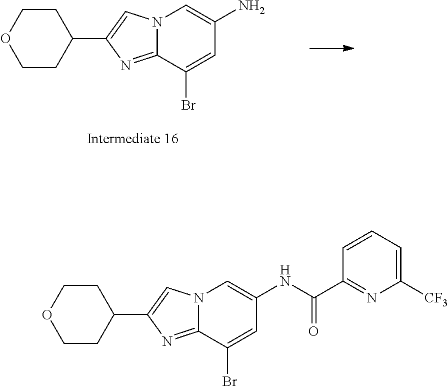 [SMILES: Nc1cc(Br)c2nc(C3CCOCC3)cn2c1 ; O=C(Nc1cc(Br)c2nc(C3CCOCC3)cn2c1)c1cccc(C(F)(F)F)n1]

Example 106: N-(8-bromo-2-(tetrahydro-2H-pyran-4-yl)imidazo[1,2-a]pyridin-6-yl)-6-(trifluoromethyl)picolinamide was prepared in a similar manner to that described for Intermediate 3c, starting from 8-bromo-2-(tetrahydro-2H-pyran-4-yl)imidazo[1,2-a]pyridin-6-amine. LCMS (ESI) m/z 471.1 (M+H)+, ¹H NMR (400 MHz, METHANOL-d₄) δ ppm 1.78-1.88 (m, 2H) 2.04 (br dd, J=13.05, 1.76 Hz, 2H) 3.01-3.09 (m, 1H) 3.58-3.64 (m, 2H) 4.02-4.09 (m, 2H) 7.82 (d, J=0.75 Hz, 1H) 7.93 (d, J=1.76 Hz, 1H) 8.07 (dd, J=7.78, 1.00 Hz, 1H) 8.30 (t, J=7.78 Hz, 1H) 8.46 (d, J=7.53 Hz, 1H) 9.24 (d, J=1.76 Hz, 1H).

### Example 107: N-(8-(3,6-dihydro-2H-pyran-4-yl)-2-(tetrahydro-2H-pyran-4-yl)imidazo[1,2-a]pyridin-6-yl)-6-(trifluoromethyl)picolinamide

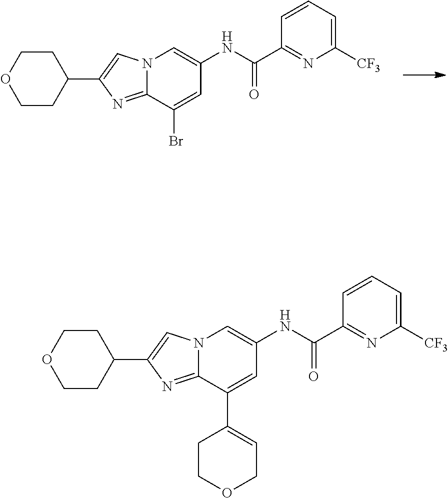 [SMILES: O=C(Nc1cc(Br)c2nc(C3CCOCC3)cn2c1)c1cccc(C(F)(F)F)n1 ; O=C(Nc1cc(C2=CCOCC2)c2nc(C3CCOCC3)cn2c1)c1cccc(C(F)(F)F)n1]

A mixture of K₂CO₃ (35 mg, 260 μmol), N-(8-bromo-2-(tetrahydro-2H-pyran-4-yl)imidazo[1,2-a]pyridin-6-yl)-6-(trifluoromethyl)picolinamide (40 mg, 85 μmol), 1,1′-bis(diphenylphosphino)ferrocene palladium(II) chloride dichloromethane complex (6.9 mg, 8.5 μmol) and 2-(3,6-dihydro-2H-pyran-4-yl)-4,4,5,5-tetramethyl-1,3,2-dioxaborolane (26.8 mg, 128 μmol) in dioxane (1 mL) was heated at 80° C. for 16 h before being quenched by the addition of water (10 mL). The layers were separated and the aqueous layer was extracted with EtOAc (3×10 mL), dried over MgSO₄, filtered, concentrated, and purified by TFA-modified mass-directed HPLC on a reverse phase column (5-95% gradient of water to acetonitrile) to obtain N-(8-(3,6-dihydro-2H-pyran-4-yl)-2-(tetrahydro-2H-pyran-4-yl)imidazo[1,2-a]pyridin-6-yl)-6-(trifluoromethyl)picolinamide 2,2,2-trifluoroacetate (20 mg, 34 μmol). LCMS (ESI) m/z 473.2 (M+H)⁺.

### Example 108: 6-(6-(difluoromethyl)picolinamido)-7-ethoxy-N,N-dimethylimidazo[1,2-a]pyridine-2-carboxamide

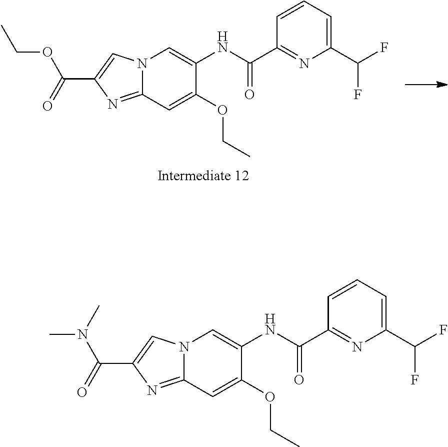 [SMILES: CCOC(=O)c1cn2cc(NC(=O)c3cccc(C(F)F)n3)c(OCC)cc2n1 ; CCOc1cc2nc(C(=O)N(C)C)cn2cc1NC(=O)c1cccc(C(F)F)n1]

In a sealed tube ethyl 6-(6-(difluoromethyl)picolinamido)-7-ethoxyimidazo[1,2-a]pyridine-2-carboxylate (Intermediate 12, 300 mg, 973 μmol) was dissolved in a methanol solution of dimethylamine (6 mL, 1 M) and heated at 80° C. for 16 h. The mixture was cooled to rt. and concentrated in vacuo. The residue was triturated with water and the precipitate formed was collected by filtration. The resulting solid was purified by HPLC (column: Waters SunFire C18 5 mkm, 19×100; ACN-water, 30 ml/min) to give 6-(6-(difluoromethyl)picolinamido)-7-ethoxy-N,N-dimethylimidazo[1,2-a]pyridine-2-carboxamide (5.2 mg, 13 μM, 1.32%) as a white solid, 100% purity by LCMS. LCMS (ESI) m/z 404.2 (M+H)⁺. ¹H NMR: (400 MHz, Methanol-d₄) δ 9.50 (s, 1H), 8.32 (d, J=7.3 Hz, 1H), 8.21 (t, J=7.6 Hz, 1H), 8.04 (s, 1H), 7.90 (d, J=7.4 Hz, 1H), 6.95 (s, 1H), 6.74 (t, J=55.1 Hz, 1H), 4.27 (q, J=6.8 Hz, 2H), 3.36 (s, 3H), 3.11 (s, 3H), 1.59 (t, J=6.8 Hz, 3H).

### Example 109: N-(2-(1,1-difluoroethyl)-7-ethoxyimidazo[1,2-a]pyridin-6-yl)-6-(difluoromethyl)picolinamide

Example 109 was prepared in a similar fashion as Example 82, except 2,2-difluoropropanoic acid was used in place of 2-(1,2,4-triazol-1-yl)acetic acid. LCMS (ESI) m/z 397.2 (M+H)⁺. ¹H NMR (400 MHz, DMSO-d₆) δ 10.53 (s, 1H), 9.53 (s, 1H), 8.32 (m, 2H), 8.19 (s, 1H), 8.01 (dd, J=6.2, 2.4 Hz, 1H), 7.17 (s, 1H), 7.12 (t, J=54.7 Hz, 1H), 4.26 (q, J=6.8 Hz, 2H), 2.01 (t, J=18.6 Hz, 3H), 1.51 (t, J=6.9 Hz, 3H).

### Example 110: 6-(difluoromethyl)-N-(2-(3,3-difluoropropyl)-7-ethoxyimidazo[1,2-a]pyridin-6-yl)picolinamide

Example 110 was prepared in a similar fashion as Example 82 except 4,4-difluorobutanoic acid was used in place of 2-(1,2,4-triazol-1-yl)acetic acid. LCMS (ESI) m/z 411 (M+H)⁺. ¹H NMR (400 MHz, DMSO-d₆) δ 10.48 (s, 1H), 9.41 (s, 1H), 8.32 (m, 2H), 8.00 (m, 1H), 7.72 (s, 1H), 7.12 (t, J=54.8 Hz, 1H), 7.06 (s, 1H), 6.17 (t, J=56.8 Hz, 1H), 4.23 (m, 2H), 2.76 (t, J=7.6 Hz, 2H), 2.20 (m, 1H), 1.49 (t, J=6.8 Hz, 3H).

### Example 111: 6-(difluoromethyl)-N-(7-ethoxy-2-(1-fluorocyclopropyl)imidazo[1,2-a]pyridin-6-yl)picolinamide

Example 111 was prepared in a similar fashion as Example 82, except 1-fluorocyclopropane-1-carboxylic acid was used in place of 2-(1,2,4-triazol-1-yl)acetic acid. LCMS (ESI) m/z 392 (M+H)⁺. ¹H NMR (400 MHz, Chloroform-d) δ 10.53 (s, 1H), 9.44 (s, 1H), 8.37 (d, J=8.0 Hz, 1H), 8.08 (d, J=7.7 Hz, 1H), 7.82 (d, J=7.9 Hz, 1H), 7.53 (s, 1H), 6.86 (s, 1H), 6.65 (t, J=55.4 Hz, 1H), 4.18 (q, J=7.0 Hz, 2H), 1.59 (t, J=7.0 Hz, 3H), 1.51-1.25 (m, 4H).

### Example 112: N-(7-ethoxy-2-(tetrahydro-2H-pyran-3-yl)imidazo[1,2-a]pyridin-6-yl)-6-(trifluoromethyl)picolinamide

Example 112 was prepared in a similar fashion as Example 82, except tetrahydro-2H-pyran-3-carboxylic acid was used in place of 2-(1,2,4-triazol-1-yl)acetic acid. LCMS (ESI) m/z 435.5 ¹H NMR (500 MHz, METHANOL-d₄) δ ppm 1.66 (t, J=7.02 Hz, 3H) 1.72-1.82 (m, 2H) 1.84-1.96 (m, 1H) 2.19-2.30 (m, 1H) 3.15 (td, J=8.70, 3.97 Hz, 1H) 3.58-3.71 (m, 2H) 3.90 (dt, J=10.99, 4.27 Hz, 1H) 4.10 (dd, J=11.60, 3.05 Hz, 1H) 4.45 (q, J=6.92 Hz, 2H) 7.26 (s, 1H) 7.94 (s, 1H) 8.13 (d, J=7.33 Hz, 1H) 8.36 (t, J=7.94 Hz, 1H) 8.51 (d, J=7.94 Hz, 1H) 9.79 (s, 1H)

### Example 113: N-(2-(2,2-dimethyltetrahydro-2H-pyran-4-yl)-7-ethoxyimidazo[1,2-a]pyridin-6-yl)-6-(trifluoromethyl)picolinamide

Example 113 was prepared in a similar fashion as Example 82: except tetrahydro-2H-pyran-3-carboxylic acid was used in place 2-(1,2,4-triazol-1-yl)acetic acid. LCMS (ESI) m/z 464.5 ¹H NMR (500 MHz, METHANOL-d₄) δ ppm 1.29 (s, 4H) 1.37 (s, 3H) 1.66 (t, J=6.71 Hz, 4H) 1.70-1.80 (m, 1H) 1.93-2.05 (m, 2H) 3.34 (br t, J=3.66 Hz, 1H) 3.81-3.94 (m, 2H) 4.45 (q, J=6.71 Hz, 2H) 7.26 (s, 1H) 7.89 (s, 1H) 8.13 (d, J=7.94 Hz, 1H) 8.36 (t, J=7.94 Hz, 1H) 8.51 (d, J=7.94 Hz, 1H) 9.78 (s, 1H).

### Examples 114 and 115: 6-(difluoromethyl)-N-(7-ethoxy-2-((1r,3r)-3-methoxy cyclobutyl)imidazo[1,2-a]pyridin-6-yl)picolinamide and 6-(difluoromethyl)-N-(7-ethoxy-2-((1s,3s)-3-methoxy cyclobutyl)imidazo[1,2-a]pyridin-6-yl)picolinamide

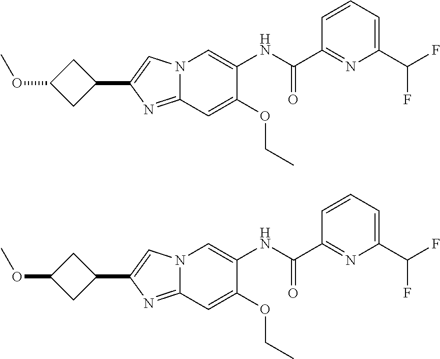

Examples 114 and 115: were prepared in a similar fashion as Example 82: except 3-methoxycyclobutane-1-carboxylic acid was used in place 2-(1,2,4-triazol-1-yl)acetic acid. Separation of the diastereomers was obtained via by chiral HPLC (CHIRALCEL OJ-H, 250 mm*30 mm, 5 mkm, Hexane-IPA-MeOH, 90-5-5, 30 mL/min).

### Example 114: 6-(difluoromethyl)-N-(7-ethoxy-2-((1r,3r)-3-methoxycyclobutyl)imidazo[1,2-a]pyridin-6-yl)picolinamide

Rt (CHIRALCEL OJ-H, Hexane-IPA-MeOH, 70-15-15, 0.6 mL/min)—17.3 min LCMS (ESI) m/z 417.2 (M+H)⁺. 1H NMR (400 MHz, DMSO-d6) δ 10.47 (s, 1H), 9.38 (s, 1H), 8.31 (m, 2H), 7.99 (m, 1H), 7.66 (s, 1H), 7.11 (t, J=54.8 Hz, 1H), 7.04 (s, 1H), 4.22 (q, J=6.8 Hz, 2H), 3.82 (p, J=7.6 Hz, 1H), 3.16 (s, 3H), 3.00 (p, J=9.5, 8.7 Hz, 1H), 2.54 (m, 2H), 2.06 (qd, J=8.4, 2.8 Hz, 2H), 1.50 (s, 3H).

### Example 115: 6-(difluoromethyl)-N-(7-ethoxy-2-((1s,3s)-3-methoxycyclobutyl)imidazo[1,2-a]pyridin-6-yl)picolinamide

Rt (CHIRALCEL OJ-H, Hexane-IPA-MeOH, 70-15-15, 0.6 mL/min)—15.8 min LCMS (ESI) m/z 417.2 (M+H)⁺. 1H NMR (400 MHz, DMSO-d6) δ 10.47 (s, 1H), 9.38 (s, 1H), 8.31 (m, 2H), 7.99 (m, 1H), 7.66 (s, 1H), 7.11 (t, J=54.8 Hz, 1H), 7.04 (s, 1H), 4.22 (q, J=6.8 Hz, 2H), 3.82 (p, J=7.6 Hz, 1H), 3.16 (s, 3H), 3.00 (p, J=9.5, 8.7 Hz, 1H), 2.54 (m, 2H), 2.06 (qd, J=8.4, 2.8 Hz, 2H), 1.50 (s, 3H).

### Example 116: 6-(difluoromethyl)-N-(7-ethoxy-2-(tetrahydro-2H-pyran-4-yl)imidazo[1,2-a]pyridin-6-yl)picolinamide

Example 116 was prepared in a similar fashion to that described for Example 12, using 2-chloro-1-(tetrahydro-2H-pyran-4-yl)ethan-1-one in place of 1-chloro-5-methoxypentan-2-one to provide 6-(difluoromethyl)-N-(7-ethoxy-2-(tetrahydro-2H-pyran-4-yl)imidazo[1,2-a]pyridin-6-yl)picolinamide. LCMS (ESI) m/z 417.2 (M+H)⁺. 1H NMR: (400 MHz, DMSO-d6) δ 10.46 (s, 1H), 9.39 (s, 1H), 8.38-8.17 (m, 2H), 8.04-7.94 (m, 1H), 7.65 (s, 1H), 7.10 (t, J=54.7 Hz, 1H), 7.05 (s, 1H), 4.22 (q, J=6.8 Hz, 2H), 3.91 (d, J=11.1 Hz, 2H), 3.50-3.40 (m, 2H), 2.85 (ddt, J=11.1, 7.5, 3.7 Hz, 1H), 1.89 (d, J=12.8 Hz, 2H), 1.67 (qd, J=12.8, 4.2 Hz, 2H), 1.48 (t, J=6.9 Hz, 3H).

### Example 117: N-(2-cyclopropyl-7-ethoxyimidazo[1,2-a]pyridin-6-yl)-6-methoxypicolinamide

A solution of 6-methoxypyridine-2-carboxylic acid (32 mg, 210 mmol) in chloroform (10 mL) containing a catalytic amount of DMF was treated with oxalyl chloride (29 mg, 230 mmol, 20 μL). The solution was maintained at rt until the gas evolution ceased (approximately 2 h) and concentrated under reduced pressure. The resulting oil was dissolved in chloroform (5 ml) and added to a solution of 2-cyclopropyl-7-ethoxy-imidazo[1,2-a]pyridin-6-amine (Intermediate 5c, 41 mg, 190 mmol) and triethylamine (57 mg, 570 μmol, 80 μL) in chloroform (10 mL). This solution was maintained at rt overnight, washed with water (5 mL), saturated aqueous NaHCO₃ solution (5 mL) and concentrated. The resulting residue was purified by HPLC to provide N-(2-cyclopropyl-7-ethoxy-imidazo[1,2-a]pyridin-6-yl)-6-methoxy-pyridine-2-carboxamide (16 mg, 43 mmol, 23% yield). LCMS (ESI) m/z 353.2 (M+H)⁺. ¹H NMR (400 MHz, Methanol-d₄): δ 9.37 (s, 1H), 7.87-7.81 (m, 1H), 7.76 (d, J=7.2 Hz, 1H), 7.37 (s, 1H), 7.00 (d, J=8.2 Hz, 1H), 6.77 (s, 1H), 4.19 (q, J=6.9 Hz, 2H), 4.02 (s, 3H), 1.98-1.86 (m, 1H), 1.51 (t, J=7.0 Hz, 3H), 0.98-0.87 (m, 2H), 0.85-0.74 (m, 2H).

### Example 118: N-(2-cyclopropyl-7-ethoxyimidazo[1,2-a]pyridin-6-yl)-6-(trifluoromethoxy)picolinamide

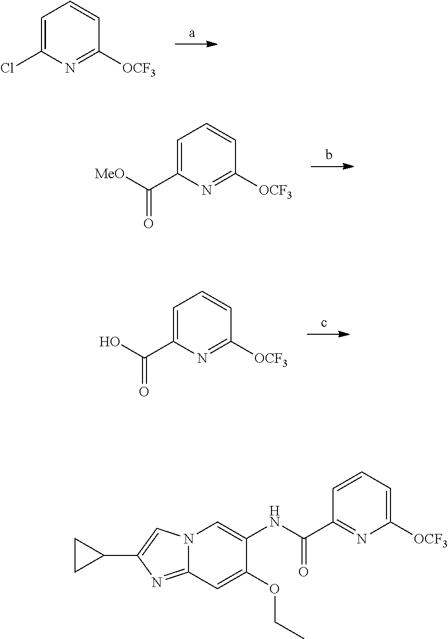 [SMILES: FC(F)(F)Oc1cccc(Cl)n1 ; COC(=O)c1cccc(OC(F)(F)F)n1 ; O=C(O)c1cccc(OC(F)(F)F)n1 ; CCOc1cc2nc(C3CC3)cn2cc1NC(=O)c1cccc(OC(F)(F)F)n1]

Step a: 2-chloro-6-(trifluoromethoxy)pyridine (500 mg, 2.53 mmol), triethylamine (307 mg, 3.04 mmol, 420 mL) and Pd(dppf)Cl₂ (93 mg, 127 μmol) were dissolved in methanol (50 mL), placed in a steel autoclave and heated at 100° C. for 24 h under carbon monoxide atmosphere (38000 Torr). Upon complete conversion (monitored by TLC), the solvent was removed, the residue was taken up in MTBE (100 mL) and filtered through a silica pad. MTBE was then evaporated to obtain methyl 6-(trifluoromethoxy)pyridine-2-carboxylate (4.50 g, crude, 90% by H NMR) as a red liquid, which was used without further purification. ¹H NMR (CDCl₃, 400 MHz): 8.05 (d, J=7.6 Hz, 1H); 7.94 (t, J=8 Hz, 1H); 7.24 (d, J=7.6 Hz, 1H); 3.98 (s, 3H).

Step b: To a solution of methyl 6-(trifluoromethoxy)pyridine-2-carboxylate (450 mg, 1.83 mmol) in THF (30 mL) was added lithium hydroxide monohydrate (76.8 mg, 1.83 mmol) as a solution in water (5 mL) as a single portion. The mixture was stirred overnight at rt. The solvents were then evaporated and the residue was dissolved in water (30 mL). This solution was washed with DCM (2×20 mL) and acidified by the addition of sodium hydrogen sulfate (880 mg, 7.3 mmol) as a solution in water (10 mL). The mixture was extracted with DCM (2×30 mL), the organic phase was dried over sodium sulfate and evaporated to give 6-(trifluoromethoxy)pyridine-2-carboxylic acid (333 mg, 1.61 mmol, 87.9% yield) as a white crystalline powder. LCMS (ESI) m/z 208.2 (M+H)⁺. ¹H NMR (CDCl₃, 500 MHz): 8.19 (d, J=7.6 Hz, 1H); 8.10 (t, J=7.9 Hz 1H); 7.34 (d, J=7.6 Hz, 1H).

Step c: 6-(trifluoromethoxy)pyridine-2-carboxylic acid (36.7 mg, 177 mmol) was dissolved in chloroform (10 mL) containing a catalytic amount of DMF and treated with oxalyl chloride (24 mg, 193 mmol, 16.4 mL). The solution obtained was stirred at rt until the gas evolution ceased (approximately 2 hours) and concentrated under reduced pressure. The resulting oil was dissolved in chloroform (5 mL) and added to the solution of 2-cyclopropyl-7-ethoxy-imidazo[1,2-a]pyridin-6-amine (35 mg, 161 mmol) and triethylamine (48.9 mg, 483 mmol, 67 mL) in chloroform (10 mL). This solution was kept at rt overnight, washed with water (5 mL), saturated aqueous NaHCO₃ (5 mL) and concentrated in vacuo. The resulting residue was purified by HPLC to provide N-(2-cyclopropyl-7-ethoxy-imidazo[1,2-a]pyridin-6-yl)-6-(trifluoromethoxy)pyridine-2-carboxamide (14 mg, 34 mmol, 20.8% yield). LCMS (ESI) m/z 407.0 (M+H)⁺. ¹H NMR: CD₃OD, 400 MHz: 9.37 (s, 1H); 8.14 (m, 2H); 7.34 (m, 2H); 6.75 (s, 1H); 4.19 (q, J=6.8 Hz, 2H), 1.93 (m, 1H); 1.50 (t, J=7.3 Hz, 3H); 0.93 (m, 2H); 0.8 (m, 2H).

### Example 119: N-(2-cyclopropyl-7-ethoxyimidazo[1,2-a]pyridin-6-yl)-2,3-dihydrobenzofuran-7-carboxamide

Example 119, was prepared in a similar fashion to that described for Example 117, using 2,3-dihydrobenzofuran-7-carboxylic acid in place of 6-methoxypyridine-2-carboxylic acid. LCMS (ESI) m/z 364.2 (M+H)⁺. ¹H NMR (400 MHz, Methanol-d₄) δ 9.67 (s, 1H), 7.80 (d, J=7.9 Hz, 1H), 7.70 (s, 1H), 7.44 (d, J=7.2 Hz, 1H), 7.09 (s, 1H), 7.00 (t, J=7.6 Hz, 1H), 4.80 (t, J=8.8 Hz, 2H), 4.34 (q, J=6.9 Hz, 2H), 3.30 (t, J=8.5 Hz, 2H), 2.11-1.99 (m, 1H), 1.65 (t, J=6.9 Hz, 3H), 1.22-1.11 (m, 2H), 0.94-0.84 (m, 2H).

### Example 120: N-(2-cyclopropyl-7-ethoxyimidazo[1,2-a]pyridin-6-yl)-6-(trifluoromethyl)pyrazine-2-carboxamide

Example 120 was prepared in a similar fashion to that described for Example 117, using 6-(trifluoromethyl)pyrazine-2-carboxylic acid in place of 6-methoxypyridine-2-carboxylic acid. LCMS (ESI) m/z 392.2 (M+H)⁺. 1H NMR (400 MHz, Methanol-d4) δ 9.63 (s, 1H), 9.40 (s, 1H), 9.33 (s, 1H), 7.45 (s, 1H), 6.86 (s, 1H), 4.25 (q, J=7.1 Hz, 2H), 2.00-1.89 (m, 1H), 1.56 (t, J=6.9 Hz, 3H), 0.96-0.89 (m, 2H), 0.84-0.76 (m, 2H).

### Example 121: 6-(difluoromethyl)-N-(7-ethoxy-2-(morpholinomethyl)imidazo[1,2-a]pyridin-6-yl)picolinamide

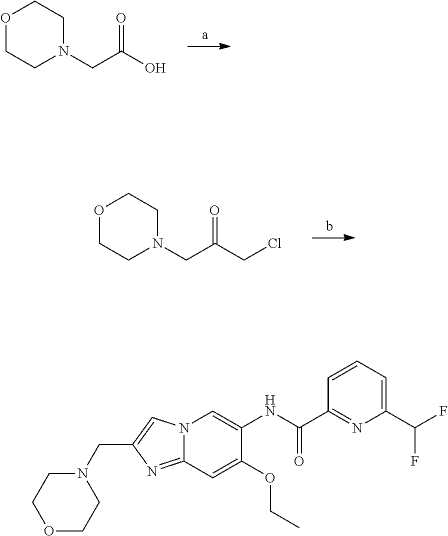 [SMILES: O=C(O)CN1CCOCC1 ; O=C(CCl)CN1CCOCC1 ; CCOc1cc2nc(CN3CCOCC3)cn2cc1NC(=O)c1cccc(C(F)F)n1]

Step a: To a solution of 2-morpholinoacetic acid hydrochloride (500 mg, 2.75 mmol) in chloroform (20 mL) was added thionyl chloride (0.66 g, 5.5 mmol) at 0° C. under N₂. The mixture was stirred at rt. for 12 h. The mixture was concentrated in vacuo to give 2-morpholinoacetyl chloride (0.55 g, 2.75 mmol), which was used immediately. The crude 2-morpholinoacetyl chloride was dissolved in THF (10 mL) and cooled to 0° C. Acetonitrile (10 mL) was added followed by an Et₂O solution of diazomethyl(trimethyl)silane (2 M, 1.6 mL). The solution was maintained at 0° C. for 1 h. Hydrochloric acid (12 M, 2 mL) was added to the mixture at 0° C. The resulting mixture was stirred at 30° C. for 3 h. The mixture was diluted with EtOAc (100 mL) then basified with saturated aqueous NaHCO₃ solution to pH=8-9. The aqueous layer was extracted with EtOAc (3×100 mL). The combined organic layers were washed with brine (100 mL), dried over Na₂SO₄, filtered and concentrated in vacuo to provide 1-chloro-3-morpholinopropan-2-one as a red-brown oil, which was used in the next step without purification. ¹H NMR (400 MHz, Chloroform-d) δ 4.45 (s, 2H), 3.73-3.64 (m, 8H), 3.56-3.52 (m, 2H).

Step b: A vial was charged with Intermediate 3c: N-(6-amino-4-ethoxypyridin-3-yl)-6-(difluoromethyl)picolinamide (300 mg, 973 μmol), 1-chloro-3-morpholinopropan-2-one (180 mg, 1.01 mmol) and NaHCO₃ (90 mg, 1.1 mmol). EtOH (6 mL) was added and the vial was sealed and heated at 80° C. for 16 h. The mixture was then cooled to rt and diluted with H₂O (25 mL) to form a red-brown precipitate, which was filtered and dried in vacuo. The resulting solid was purified by HPLC (column SunFire C18 100*19 mm, 5 mkm, ACN-water 30 mL/min) to yield 6-(difluoromethyl)-N-(7-ethoxy-2-(morpholinomethyl)imidazo[1,2-a]pyridin-6-yl)picolinamide (40.1 mg, 100 μM, 9.5% yield). LCMS (ESI) m/z 432.2 (M+H)⁺. ¹H NMR (400 MHz, Chloroform-d) δ 10.54 (s, 1H), 9.33 (s, 1H), 8.38 (d, J=7.8 Hz, 1H), 8.09 (t, J=7.8 Hz, 1H), 7.82 (d, J=7.8 Hz, 1H), 6.76 (s, 1H), 6.65 (t, J=55.3 Hz, 1H), 4.17 (q, J=6.9 Hz, 2H), 3.91 (t, J=4.5 Hz, 4H), 3.14 (br. s, 4H), 2.44 (s, 3H), 1.58 (t, J=6.9 Hz, 3H).

### Example 122: N-(7-ethoxy-2-((tetrahydrofuran-3-yl)methyl)imidazo[1,2-a]pyridin-6-yl)-1-isopropyl-1H-pyrazole-3-carboxamide

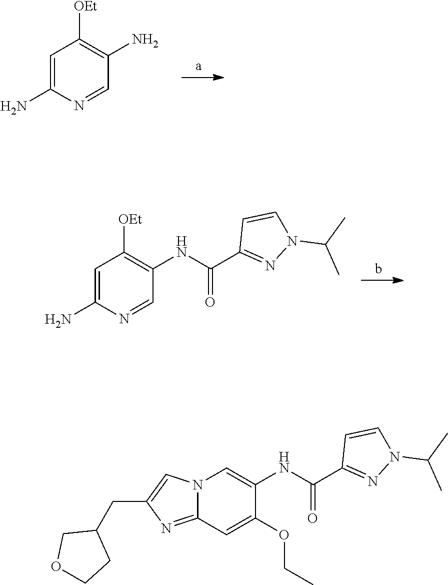 [SMILES: CCOc1cc(N)ncc1N ; CCOc1cc(N)ncc1NC(=O)c1ccn(C(C)C)n1 ; CCOc1cc2nc(CC3CCOC3)cn2cc1NC(=O)c1ccn(C(C)C)n1]

Step a: HATU (538 mg, 1.41 mmol), DIPEA (456 mg, 3.53 mmol, 620 μL) and compound 1-isopropylpyrazole-3-carboxylic acid (163 mg, 1.06 mmol) were added to a solution of Intermediate 3a, 4-ethoxypyridine-2,5-diamine (250 mg, 1.18 mmol) in DMF (3 mL). The mixture was stirred at 30° C. for 3 h then diluted with H₂O (50 mL) and extracted with EtOAc (4×50 mL). The combined EtOAc phases were washed with H₂O (50 mL) and brine (50 mL), dried over Na₂SO₄, filtered and concentrated in vacuo to provide N-(6-amino-4-ethoxy-3-pyridyl)-1-isopropyl-pyrazole-3-carboxamide as a grey solid, which was used in the next step without further purification assuming quantitative yield. LCMS (ESI) m/z 290.2 (M+H)⁺.

Step b: A vial was charged with N-(6-amino-4-ethoxy-3-pyridyl)-1-isopropyl-pyrazole-3-carboxamide (290 mg, 1.00 mmol), 1-chloro-3-tetrahydrofuran-3-yl-propan-2-one (381 mg, 3.00 mmol) and NaHCO₃ (253 mg, 3.00 mmol). EtOH (15 mL) was added and the vial was sealed and heated at 80° C. for 16 h. The mixture was cooled to room temperature, diluted with H₂O (25 mL) and extracted with EtOAc (4×25 mL). The combined organic layers were washed with H₂O (50 mL) and brine (50 mL), dried over Na₂SO₄, filtered and concentrated in vacuo. The resulting brown solid was purified by HPLC to give N-[7-ethoxy-2-(tetrahydrofuran-3-ylmethyl)imidazo[1,2-a]pyridin-6-yl]-1-isopropyl-pyrazole-3-carboxamide (78 mg, 0.2 mmol, 19.6% yield). LCMS (ESI) m/z 398.2 (M+H)⁺. ¹H NMR (400 MHz, DMSO-d₆): δ 9.34 (s, 1H), 9.23 (s, 1H), 7.97 (d, J=2.4 Hz, 1H), 7.62 (s, 1H), 6.99 (s, 1H), 6.77 (d, J=2.4 Hz, 1H), 4.64-4.56 (m, 1H), 4.20 (q, J=6.9 Hz, 2H), 3.80-3.70 (m, 2H), 3.66-3.58 (m, 1H), 3.40 (dd, J=8.3, 6.2 Hz, 1H), 2.65 (d, J=7.3 Hz, 2H), 2.57 (dt, J=13.6, 7.3 Hz, 1H), 2.01-1.89 (m, 1H), 1.63-1.54 (m, 1H), 1.53-1.34 (m, 9H).

### Example 123: N-(7-ethoxy-2-((tetrahydrofuran-3-yl)methyl)imidazo[1,2-a]pyridin-6-yl)-1-(2,2,2-trifluoroethyl)-1H-pyrazole-3-carboxamide

Example 123 was prepared in a similar fashion to that described for Example 122, using 1-(2,2,2-trifluoroethyl)pyrazole-3-carboxylic acid in place of 1-isopropylpyrazole-3-carboxylic acid. LCMS (ESI) m/z 438.4 (M+H)⁺. ¹H NMR (400 MHz, DMSO-d₆): δ 9.26 (s, 1H), 9.19 (s, 1H), 8.06 (d, J=2.5 Hz, 1H), 7.63 (s, 1H), 7.00 (s, 1H), 6.90 (d, J=2.4 Hz, 1H), 5.31 (q, J=9.0 Hz, 2H), 4.20 (q, J=6.9 Hz, 2H), 3.81-3.69 (m, 2H), 3.67-3.56 (m, 1H), 3.40 (dd, J=8.4, 6.4 Hz, 1H), 2.66 (d, J=7.2 Hz, 2H), 2.57 (dd, J=14.3, 7.2 Hz, 1H), 1.97 (dt, J=12.6, 6.4 Hz, 1H), 1.60 (dt, J=12.6, 6.9 Hz, 1H), 1.42 (t, J=6.9 Hz, 3H).

### Example 124: 1-(difluoromethyl)-N-(7-ethoxy-2-((tetrahydrofuran-3-yl)methyl)imidazo[1,2-a]pyridin-6-yl)-1H-pyrazole-3-carboxamide

Example 124 was prepared in a similar fashion to that described for Example 122, using 1-(difluoromethyl)-1H-pyrazole-3-carboxylic acid in place of 1-isopropylpyrazole-3-carboxylic acid. LCMS (ESI) m/z 406.2 (M+H)⁺. ¹H NMR (400 MHz, DMSO-d₆) δ 9.36 (s, 1H), 9.10 (s, 1H), 8.45 (d, J=2.7 Hz, 1H), 7.95 (t, J=58.7 Hz, 1H), 7.62 (s, 1H), 7.03 (d, J=2.7 Hz, 1H), 7.00 (s, 1H), 4.20 (q, J=7.0 Hz, 2H), 3.81-3.70 (m, 2H), 3.63 (q, J=7.6 Hz, 1H), 3.40 (dd, J=8.2, 6.2 Hz, 1H), 2.67 (d, J=7.2 Hz, 2H), 2.57 (q, J=7.2 Hz, 1H), 2.06-1.92 (m, 1H), 1.67-1.55 (m, 1H), 1.41 (t, J=6.9 Hz, 3H).

### Example 125: 1-(2,2-difluoroethyl)-N-(7-ethoxy-2-((tetrahydrofuran-3-yl)methyl)imidazo[1,2-a]pyridin-6-yl)-1H-pyrazole-3-carboxamide

Example 125 was prepared in a similar fashion to that described for Example 122, using 1-(2,2-difluoroethyl)-1H-pyrazole-3-carboxylic acid in place of 1-isopropylpyrazole-3-carboxylic acid. LCMS (ESI) m/z 420.2 (M+H)⁺. ¹H NMR (400 MHz, DMSO-d₆) δ 9.33 (d, J=10.0 Hz, 2H), 8.01 (d, J=2.4 Hz, 1H), 7.82 (s, 1H), 7.13 (s, 1H), 6.88 (d, J=2.4 Hz, 1H), 6.70-6.21 (m, 1H), 4.79 (td, J=15.2, 3.5 Hz, 2H), 4.29 (q, J=6.9 Hz, 2H), 3.79-3.70 (m, 2H), 3.65 (q, J=7.6 Hz, 1H), 3.40 (dd, J=8.4, 6.2 Hz, 1H), 2.73 (d, J=7.4 Hz, 2H), 2.59 (q, J=7.1 Hz, 1H), 2.06-1.90 (m, 1H), 1.60 (dq, J=13.5, 7.1 Hz, 1H), 1.45 (t, J=6.9 Hz, 3H).

### Example 126: 6-(difluoromethyl)-N-(7-ethoxy-2-(morpholine-4-carbonyl)imidazo[1,2-a]pyridin-6-yl)picolinamide

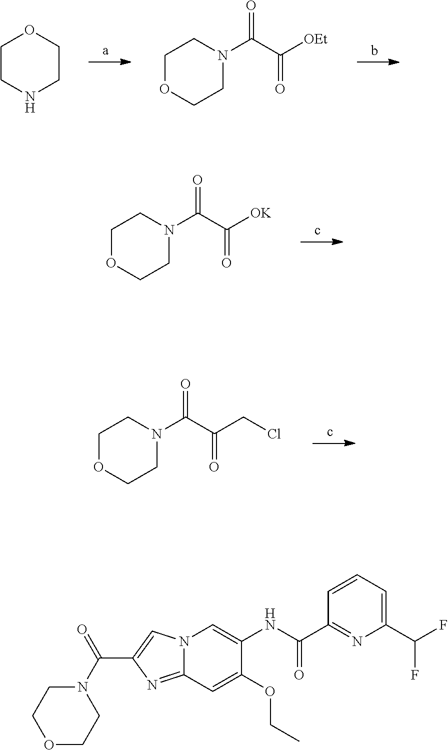

Step a: A solution of ethyl 2-chloro-2-oxoacetate (1.42 g, 10.4 mmol) in dichloromethane (5 mL) was added dropwise to a solution of morpholine (1.0 g, 11.5 mmol) and triethylamine (1.16 g, 11.5 mmol, 1.6 mL) in dichloromethane (20 mL) at 0° C. The reaction mixture was allowed to warm to rt and stirred for 12 h. The resulting mixture was filtered and the filtrate was concentrated under reduced pressure. The resulting yellow-colored residue was poured into 25 mL of water and extracted with ethyl acetate (3×20 mL). Combined organic layers were washed with brine, dried over anhydrous sodium sulphate, and concentrated under reduced pressure to give ethyl 2-morpholino-2-oxoacetate (1.7 g, 9.1 mmol, yield 87%), which was used for the next step without any purification. ¹H NMR (400 MHz, Chloroform-d) δ 4.41-4.09 (m, 2H), 3.77-3.46 (m, 6H), 3.50-3.24 (m, 2H), 1.33 (t, J=7.1 Hz, 3H).

Step b: The ethyl 2-morpholino-2-oxoacetate (1.7 g, 9.1 mmol) was dissolved in EtOH (20 mL), and H₂O (10 mmol) was added. The mixture was heated to 60° C., and a solution of t-BuOK (10 mmol) in EtOH (10 mL) was added dropwise over 30 min. When the addition was complete, the reaction mixture was stirred at 60° C. for 10 h. The solvent was removed in vacuo and the resulting white solid was purified by recrystallization using a mixture of EtOH and Et₂O to afford potassium 2-morpholino-2-oxoacetate (1.6 g, 8.1 mmol, 89%) as a white solid. ¹H NMR 1H NMR (400 MHz, DMSO-D6) δ 3.82-3.71 (m, 4H), 3.62-3.54 (m, 2H), 3.52-3.45 (m, 2H).

Step b: To a suspension of potassium 2-morpholino-2-oxoacetate (1.6 g, 8.1 mmol) in chloroform (20 mL), was added oxalyl chloride (2.06 g, 16.2 mmol) at 0° C. under N₂. The mixture was stirred at rt for 4 h. The mixture was then concentrated in vacuo to give 2-morpholino-2-oxoacetyl chloride, which was immediately dissolved in THF (10 mL) and ACN (10 mL). The solution was cooled to 0° C. and an Et₂O solution of diazomethyl(trimethyl)silane (2 M, 3.6 mL) was added. The mixture was stirred at 0° C. for 1 h at which time concentrated hydrochloric acid (12 M, 5 mL) was added to the mixture at 0° C. The mixture was allowed to warm to rt and maintained 2 h, at which time it was diluted with EtOAc (50 mL) and then basified with saturated aqueous NaHCO₃ solution to pH=8-9. The layers were separated and the aqueous layer was extracted with EtOAc (3×50 mL). The combined organic layers were washed with brine (100 mL), dried over Na₂SO₄, filtered and concentrated in vacuo to provide 3-chloro-1-morpholinopropane-1,2-dione (400 mg, 3.14 mmol, 33.98% yield) as a red-brown oil, which was used in the next step without further manipulation.

Step c: A vial was charged with Intermediate 3c: N-(6-amino-4-ethoxypyridin-3-yl)-6-(difluoromethyl)picolinamide (215 mg, 0.7 mmol), 3-chloro-1-morpholinopropane-1,2-dione (400 mg, 2.1 mmol) and NaHCO₃ (175 mg, 2.1 mmol). EtOH (6 mL) was added and the vial was sealed and heated at 80° C. for 16 h. The mixture was cooled to rt than diluted with H₂O (25 mL). A red-brown precipitate was formed, which was collected by vacuum filtration. The solid was purified by HPLC (column: SunFire C18, 100×19 mm, 5 um; mobile phase; 50-90% water-methanol, 1.3-5.3 min, flow 30 ml/min) to provide 6-(difluoromethyl)-N-(7-ethoxy-2-(morpholine-4-carbonyl)imidazo[1,2-a]pyridin-6-yl)picolinamide (58 mg, 0.10 mmol, 19% yield). LCMS (ESI) m/z 436.0 (M+H)⁺. ¹H NMR (500 MHz, Chloroform-d) δ 10.59 (s, 1H), 9.50 (s, 1H), 8.39 (d, J=7.7 Hz, 1H), 8.12 (t, J=7.7 Hz, 1H), 8.01 (s, 1H), 7.86 (s, 1H), 6.89 (s, 1H), 6.68 (t, J=55.3 Hz, 1H), 4.37 (br. s, 2H), 4.23 (q, J=6.9 Hz, 2H), 3.79 (br. s, 6H), 1.63 (t, J=6.9 Hz, 3H).

### Example 127: 6-(difluoromethyl)-N-(7-ethoxy-2-(3-fluorobicyclo[1.1.1]pentan-1-yl)imidazo[1,2-a]pyridin-6-yl)picolinamide

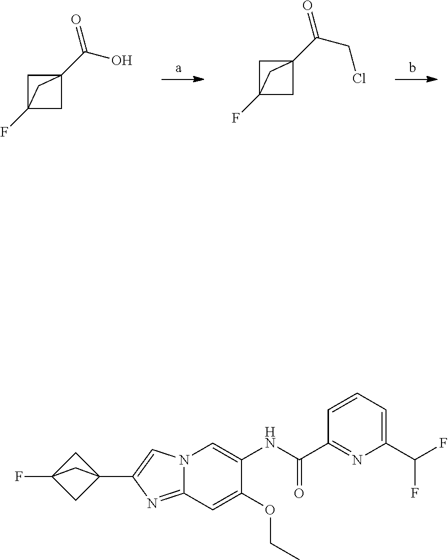 [SMILES: O=C(O)C12CC(F)(C1)C2 ; O=C(CCl)C12CC(F)(C1)C2 ; CCOc1cc2nc(C34CC(F)(C3)C4)cn2cc1NC(=O)c1cccc(C(F)F)n1]

Step a: 3-fluorobicyclo[1.1.1]pentane-1-carboxylic acid (1.00 g, 7.69 mmol) was dissolved in DCM (10 mL). Oxalyl chloride (1.07 g, 8.46 mmol) was added and the mixture was stirred at rt. for 17 h. The solution was immersed in an ice bath and acetonitrile (10 mL) was added, followed by an Et₂O solution of diazomethyl(trimethyl)silane (2M, 4.2 mL), which was introduced cautiously. The solution was allowed to warm to rt and maintained overnight. Concentrated hydrochloric acid (3 eq) was added dropwise and the resulting mixture was stirred at rt for 3 h. The reaction mixture was neutralized with saturated aqueous NaHCO₃ until no CO₂ evolution was observed. The mixture was filtered and the organic layer of the filtrate was obtained and concentrated in vacuo. The residue was distilled at 15 mm Hg (b.p. 82° C.), to give 2-chloro-1-(3-fluorobicyclo[1.1.1]pentan-1-yl)ethan-1-one as a colorless liquid (500 mg, 2.31 mmol, 30% yield, 75% purity by H NMR). 1H NMR (500 MHz, Chloroform-d) δ 4.20 (s, 2H), 2.46-2.43 (m, 6H).

Step b: Intermediate 3c: N-(6-amino-4-ethoxypyridin-3-yl)-6-(difluoromethyl)picolinamide (284 mg, 0.920 mmol), and 2-chloro-1-(3-fluorobicyclo[1.1.1]pentan-1-yl)ethan-1-one (300 mg, 1.38 mmol) were heated under reflux in propionitrile (20 mL) for 24 h. Saturated aqueous Na₂CO₃ (3 mL) was added and the mixture was stirred for 3 h. The organic phase was separated and concentrated under reduced pressure. The resulting residue was purified by HPLC to obtain 6-(difluoromethyl)-N-(7-ethoxy-2-(3-fluorobicyclo[1.1.1]pentan-1-yl)imidazo[1,2-a]pyridin-6-yl)picolinamide (19 mg, 44 μmol, 4.9% yield). LCMS (ESI) m/z 417.2 (M+H)⁺. 1H NMR (400 MHz, DMSO-d6): δ 10.47 (s, 1H), 9.40 (s, 1H), 8.36-8.28 (m, 2H), 8.00 (dd, J=5.3, 3.6 Hz, 1H), 7.75 (s, 1H), 7.28-6.90 (m, 2H), 4.21 (q, J=6.9 Hz, 2H), 2.34 (d, J=2.6 Hz, 6H), 1.49 (t, J=6.9 Hz, 3H).

### Example 128: 6-(difluoromethyl)-N-(7-ethoxy-2-(1-methoxycyclopropyl)imidazo[1,2-a]pyridin-6-yl)picolinamide

Example 128 was prepared in a similar fashion to that described for Example 127: using 1-methoxycyclopropane-1-carboxylic acid in place of 3-fluorobicyclo[1.1.1]pentane-1-carboxylic acid. LCMS (ESI) m/z 403.2 (M+H)⁺. 1H NMR (400 MHz, Methanol-d4) δ 9.36 (s, 1H), 8.26 (d, J=7.8 Hz, 1H), 8.16 (t, J=7.8 Hz, 1H), 7.86 (d, J=7.8 Hz, 1H), 7.61 (s, 1H), 6.96-6.58 (m, 2H), 4.18 (q, J=7.0 Hz, 2H), 3.38 (s, 3H), 1.54 (t, J=7.0 Hz, 3H), 1.21-1.06 (m, 4H).

### Example 129: 6-(difluoromethyl)-N-(7-ethoxy-2-(3-(2-hydroxypropan-2-yl)bicyclo[1.1.1]pentan-1-yl)imidazo[1,2-a]pyridin-6-yl)picolinamide

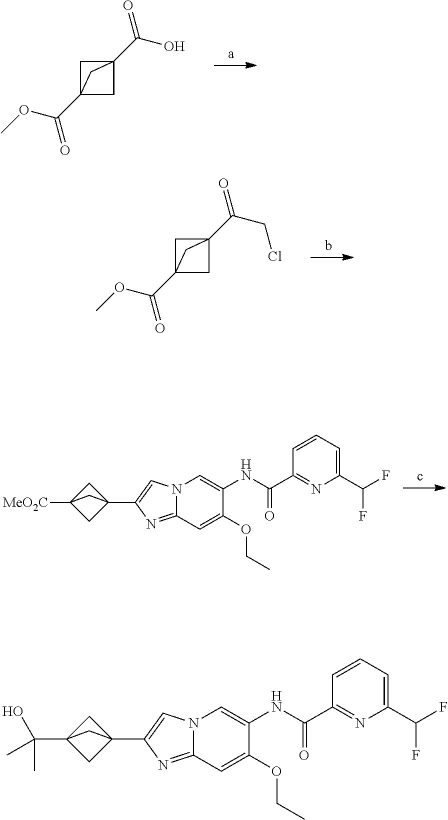 [SMILES: COC(=O)C12CC(C(=O)O)(C1)C2 ; COC(=O)C12CC(C(=O)CCl)(C1)C2 ; CCOc1cc2nc(C34CC(C(=O)OC)(C3)C4)cn2cc1NC(=O)c1cccc(C(F)F)n1 ; CCOc1cc2nc(C34CC(C(C)(C)O)(C3)C4)cn2cc1NC(=O)c1cccc(C(F)F)n1]

Step a: 3-(methoxycarbonyl)bicyclo[1.1.1]pentane-1-carboxylic acid (2.00 g, 11.8 mmol) and oxalyl chloride (1.64 g, 12.9 mmol) were mixed in DCM (5 mL). One drop of DMF was added and the mixture was stirred overnight. The solvent was removed under reduced pressure to yield methyl 3-(chlorocarbonyl)bicyclo[1.1.1]pentane-1-carboxylate (2.40 g), which was immediately taken up in a mixture of acetonitrile (5 mL) and THF (5 mL). An Et₂O solution of diazomethyl(trimethyl)silane (2 M, 5.8 mL) was added dropwise at 0° C. and the mixture was stirred at rt overnight. To this solution was added 33% aqueous HCl (3 eq) and the stirring continued for 4 h at rt, then NaHCO₃ (5 eq) was added and the resulting mixture was stirred for additional 1 h. The reaction mixture was filtered and the mother liquor was concentrated under reduced pressure to yield an oil, which was extracted with hexane (15 mL). The solvent was removed under reduced pressure to yield methyl 3-(2-chloroacetyl)bicyclo[1.1.1]pentane-1-carboxylate (1.80 g, 4.44 mmol, 41.9% yield, 50% purity by ¹H NMR), which was used without further purification. 1H NMR (400 MHz, Chloroform-d) δ 4.21-4.07 (2H), 3.68 (3H), 2.39-2.26 (6H).

Step b: Intermediate 3c: N-(6-amino-4-ethoxypyridin-3-yl)-6-(difluoromethyl)picolinamide (250 mg, 811 μmol), 3-(2-chloroacetyl)bicyclo[1.1.1]pentane-1-carboxylate (400 mg, 987 μmol) were heated to reflux in propionitrile (20 mL) for 24 h. The mixture was cooled and saturated aqueous Na₂CO₃ (10 ml) was added and the mixture was stirred at rt for 3 h. The organic phase was separated and concentrated under reduced pressure and purified by HPLC to yield methyl 3-(6-(6-(difluoromethyl)picolinamido)-7-ethoxyimidazo[1,2-a]pyridin-2-yl)bicyclo[1.1.1]pentane-1-carboxylate (120 mg, 0.3 mmol). LCMS (ESI) m/z 457.2 (M+H)⁺.

Step c: A vial was charged with methyl 3-(6-(6-(difluoromethyl)picolinamido)-7-ethoxyimidazo[1,2-a]pyridin-2-yl)bicyclo[1.1.1]pentane-1-carboxylate (50 mg, 109 μmol), sealed with a septum cap and placed under argon atmosphere. THF (10 mL) was added, followed by a prepared solution of chloro methyl magnesium (diluted with THF to 1 mL, from 0.4 mL of 3M solution in THF), which was added dropwise. The resulting mixture was stirred for 24 h and then quenched by the addition of acetic acid (0.1 mL) and water (0.2 mL) with stirring. The organic phase was separated, concentrated under reduced pressure to afford an oil which was purified by HPLC to yield 6-(difluoromethyl)-N-(7-ethoxy-2-(3-(2-hydroxypropan-2-yl)bicyclo[1.1.1]pentan-1-yl)imidazo[1,2-a]pyridin-6-yl)picolinamide (7.8 mg, 17 μmol). LCMS (ESI) m/z 457.2 (M+H)⁺. 1H NMR (400 MHz, Chloroform-d) δ 10.52 (s, 1H), 9.39 (s, 1H), 8.36 (d, J=7.7 Hz, 1H), 8.09 (t, J=7.7 Hz, 1H), 7.82 (d, J=7.7 Hz, 1H), 7.24 (s, 1H), 6.90 (s, 1H), 4.17 (q, J=7.2 Hz, 2H), 2.03 (m, 9H), 1.23 (m, 6H).

### Example 130: 6-(difluoromethyl)-N-(7-ethoxy-2-(3-(hydroxymethyl)bicyclo[1.1.1]pentan-1-yl)imidazo[1,2-a]pyridin-6-yl)picolinamide

methyl 3-(6-(6-(difluoromethyl)picolinamido)-7-ethoxyimidazo[1,2-a]pyridin-2-yl)bicyclo[1.1.1]pentane-1-carboxylate (40.0 mg, 87.6 μmol) was dissolved in THF (2 mL) and cooled to −20° C. A solution of LiAlH₄ (30.00 mg, 790.42 μmol) in diethyl ether (4 mL) was added maintaining the temperature around −10° C. The mixture was stirred for 10 min, quenched by the addition of aqueous NaOH. The layers were separated and the organic solution was concentrated under reduced pressure to yield a crude residue, which was subjected to HPLC to yield pure 6-(difluoromethyl)-N-(7-ethoxy-2-(3-(hydroxymethyl)bicyclo[1.1.1]pentan-1-yl)imidazo[1,2-a]pyridin-6-yl)picolinamide (2.8 mg, 6.54 μmol, 7.46% yield, 100% purity). LCMS (ESI) m/z 429.0 (M+H)⁺. 1H NMR (400 MHz, Methanol-d4) δ 9.39 (s, 1H), 8.31 (d, J=7.8 Hz, 1H), 8.21 (t, J=7.8 Hz, 1H), 7.89 (d, J=7.8 Hz, 1H), 7.44 (s, 1H), 6.99-6.62 (m, 2H), 4.23 (q, J=6.9 Hz, 2H), 3.60 (s, 2H), 2.03 (s, 6H), 1.57 (t, J=6.9 Hz, 3H).

### Example 131: N-(2-cyclopropyl-7-ethoxyimidazo[1,2-a]pyridin-6-yl)-6-morpholinopicolinamide

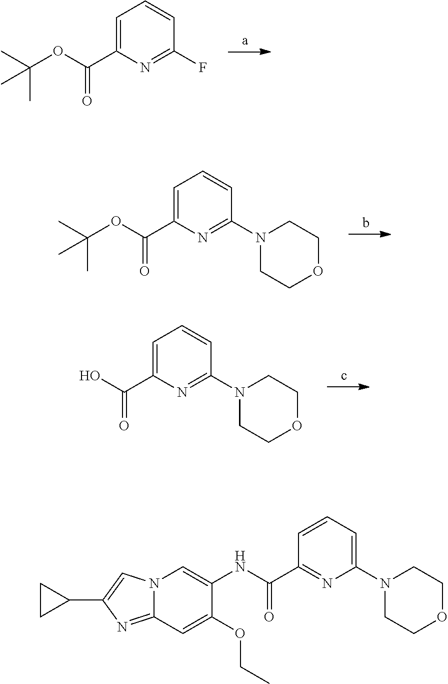 [SMILES: CC(C)(C)OC(=O)c1cccc(F)n1 ; CC(C)(C)OC(=O)c1cccc(N2CCOCC2)n1 ; O=C(O)c1cccc(N2CCOCC2)n1 ; CCOc1cc2nc(C3CC3)cn2cc1NC(=O)c1cccc(N2CCOCC2)n1]

Step a: tert-Butyl 6-fluoropyridine-2-carboxylate (545 mg, 2.76 mmol), cesium carbonate (1.80 g, 5.53 mmol) and morpholine (361 mg, 4.15 mmol, 361 μL) were heated at 100° C. in N-methylpyrrolidone (10 mL) overnight. After cooling to room temperature, the mixture was diluted with water (50 mL) and extracted with DCM (2×25 mL). The organic phase was dried over sodium sulphate and evaporated to yield crude tert-butyl 6-morpholinopyridine-2-carboxylate (400 mg, 1.36 mmol, 49% yield), which was used without further purification. ¹H NMR: (500 MHz, CDCl₃): 7.58 (t, J=8.0 Hz, 1H); 7.39 (d, J=7.5 Hz, 1H); 6.77 (d, J=8.5 Hz, 1H); 3.83 (t, J=4.5 Hz, 4H); 3.59 (t, J=4.5 Hz, 4H); 1.61 (s, 9H).

Step b: tert-Butyl 6-morpholinopyridine-2-carboxylate 6 (400 mg, 1.51 mmol) was added in 10% solution of HCl in Dioxane (30 mL). Catalytic amounts of water were added and the solution was kept at room temperature overnight. Afterwards, the solvents were evaporated and the residue was recrystallized from CH₃CN. The crystals obtained were filtered, washed with cold CH₃CN and dried to give 6-morpholinopyridine-2-carboxylic acid HCl (190 mg, 780 μmol, 51.6% yield). ¹H NMR: (500 MHz, DMSO-d₆): 7.70 (t, J=8.5 Hz, 1H); 7.33 (d, J=7.0 Hz, 1H); 7.06 (d, J=8.5 Hz, 1H); 3.69 (t, J=4.5 Hz, 4H); 3.51 (t, J=4.5 Hz, 4H).

Step c: A vial was charged with 6-morpholinopyridine-2-carboxylic acid hydrochloride (64.6 mg, 265 μmol), HATU (117.9 mg, 309.29 μmol), DIPEA (114.2 mg, 883.68 μmol, 154.3 μL), 2-cyclopropyl-7-ethoxy-imidazo[1,2-a]pyridin-6-amine (48.0 mg, 221 μmol) and DMF (10 mL). The vial was capped and heated at 40° C. overnight. After this period, the volatiles were evaporated, and the residue was dissolved in 10 mL of DCM. This solution was washed with water (5 mL) and sodium bicarbonate solution (5 ml) and evaporated to dryness. The residue was purified by HPLC to yield N-(2-cyclopropyl-7-ethoxy-imidazo[1,2-a]pyridin-6-yl)-6-morpholino-pyridine-2-carboxamide (16.0 mg, 39.3 μmol, 17.8% yield). LCMS (ESI) m/z 408.2 (M+H)⁺. ¹H NMR (400 MHz, CDCl₃): 10.36 (s, 1H); 9.42 (s, 1H); 7.68 (t, J=7.5 Hz, 1H); 7.61 (d, J=7.5 Hz 1H); 7.19 (s, 1H); 6.81 (m, 2H); 4.11 (q, J=6.8, Hz, 2H), 3.84 (t, J=4.2 Hz, 4H); 3.57 (t, J=4.2 Hz, 4H); 1.95 (m, 1H); 1.49 (t, J=6.8 Hz, 3H); 0.89 (m, 4H).

### Example 132: N-(2-cyclopropyl-7-ethoxyimidazo[1,2-a]pyridin-6-yl)-6-(1-hydroxycyclobutyl)picolinamide

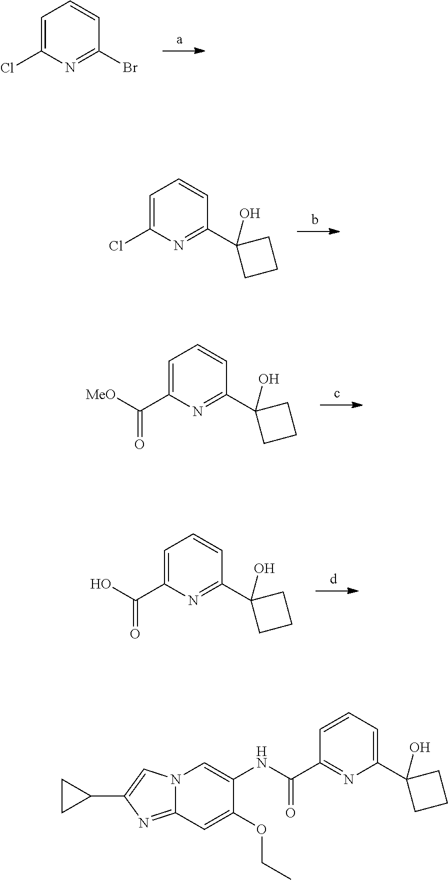 [SMILES: Clc1cccc(Br)n1 ; OC1(c2cccc(Cl)n2)CCC1 ; COC(=O)c1cccc(C2(O)CCC2)n1 ; O=C(O)c1cccc(C2(O)CCC2)n1 ; CCOc1cc2nc(C3CC3)cn2cc1NC(=O)c1cccc(C2(O)CCC2)n1]

Step a: 2-bromo-6-chloro-pyridine (10.6 g, 55.0 mmol) was dissolved in THF (800 mL) under argon and cooled to −80° C. A THF solution of butyllithium (3.87 g, 60.5 mmol, 1 M) was added slowly keeping the temperature below −70° C. The solution was stirred at this temperature for 2 h. Over this period, the color of the mixture changed from yellow to dark red. Cyclobutanone (4.62 g, 66.0 mmol, 4.91 mL) was added to the mixture dropwise, and the resulting solution was stirred at room temperature for 6 h. The reaction was quenched by the addition of saturated aqueous NH₄Cl solution. The organic phase was separated and evaporated. The residue was purified by silica gel chromatography to yield 1-(6-chloro-2-pyridyl)cyclobutanol (2.85 g, 15.5 mmol, 28.2% yield). ¹H NMR: (500 MHz, CDCl₃): 7.81 (t, J=8.0 Hz, 1H); 7.53 (d, J=7.5 Hz, 1H); 7.34 (d, J=7.5 Hz, 1H); 5.84 (s, 1H); 2.49 (m, 2H); 2.19 (m, 2H); 1.89 (m, 1H); 1.79 (m, 1H).

Step b: 1-(6-chloro-2-pyridyl)cyclobutanol (1.00 g, 5.45 mmol), triethylamine (661 mg, 6.54 mmol, 910 μL), Pd(dppf)Cl₂ (199 mg, 273 μmol) and methanol (100 mL) were placed in a steel autoclave and heated at 120° C. for 36 h under carbon monoxide atmosphere (38000 Torr). Upon the completion of conversion (monitored by TLC), the solvent was removed, the residue was treated by MTBE and filtered through the silica pad. The solvent was evaporated to obtain methyl 6-(1-hydroxycyclobutyl)pyridine-2-carboxylate (700 mg, 3.38 mmol, 62% yield) as a reddish oil, which was used in the next step without further purification. ¹H NMR: (500 MHz, CDCl₃): 8.03 (t, J=8.0 Hz, 1H); 7.92 (d, J=7.5 Hz, 1H); 7.79 (d, J=7.5 Hz, 1H); 5.18 (s, 1H); 3.99 (s, 3H); 2.60 (m, 2H); 2.51 (m, 2H); 2.12 (m, 1H); 1.92 (m, 1H).

Step c: To a solution of methyl 6-(1-hydroxycyclobutyl)pyridine-2-carboxylate (700 mg, 3.38 mmol) in THF (60 mL) was added 10 mL of an aqueous solution of lithium hydroxide monohydrate (428 mg, 10.2 mmol) as a single portion. The mixture was stirred overnight at room temperature. The solvents were then evaporated and the residue was dissolved in water (30 mL). This solution was washed with DCM (2×20 mL) and acidified by the addition of sodium hydrogen sulfate (3.27 g, 27.2 mmol) in water (10 mL). The mixture was extracted with DCM (2×30 mL), the layers were separated and the combined organic phases were dried with sodium sulfate and concentrated to give 6-(1-hydroxycyclobutyl)pyridine-2-carboxylic acid (323 mg, 1.67 mol, 49.5% yield) as white crystalline powder, which was used in the next step without further purification. ¹H NMR: (500 MHz, CDCl₃): 8.17 (t, J=8.0 Hz, 1H); 8.01 (d, J=7.5 Hz, 1H); 7.90 (d, J=7.5 Hz, 1H); 7.25-6.3 (broad s, 2H); 2.63 (m, 2H); 2.54 (m, 2H); 2.15 (m, 1H); 1.90 (m, 1H)

Step d: A vial was charged with 6-(1-hydroxycyclobutyl)pyridine-2-carboxylic acid (80.9 mg, 419 μmol, Chloride), HATU (172 mg, 451 μmol), DIPEA (125 mg, 966 μmol, 170 μL) and 2-cyclopropyl-7-ethoxy-imidazo[1,2-a]pyridin-6-amine (70.0 mg, 322 μmol) and DMF (10 mL) was added. The vial was capped and heated at 40° C. overnight. After this period, the volatiles were evaporated and the residue was dissolved in DCM (10 mL). The solution was washed with water (5 mL) and saturated aqueous sodium bicarbonate solution (5 mL) and evaporated to dryness. The residue was purified by HPLC to yield N-(2-cyclopropyl-7-ethoxy-imidazo[1,2-a]pyridin-6-yl)-6-(1-hydroxycyclobutyl)pyridine-2-carboxamide (7.0 mg, 18 μmol, 5.5% yield). LCMS (ESI) m/z 393.0 (M+H)⁺. ¹H NMR: (500 MHz, CDCl₃): 10.47 (s, 1H); 9.39 (s, 1H); 8.16 (d, J=6.5 Hz, 1H); 7.96 (t, J=6.5 Hz, 1H); 7.77 (d, J=6.5 Hz, 1H); 7.24 (s, 1H); 7.17 (s, 1H); 6.80 (s, 1H); 4.12 (q, J=6.8 Hz, 2H); 2.57 (m, 2H); 2.45 (m, 2H); 2.10 (m, 2H); 1.91 (m, 2H); 1.53 (t, J=7.3 Hz, 3H), 0.89 (m, 4H).

### Example 133: N-(7-ethoxy-2-(tetrahydro-2H-pyran-4-yl)imidazo[1,2-a]pyridin-6-yl)-6-methoxypicolinamide

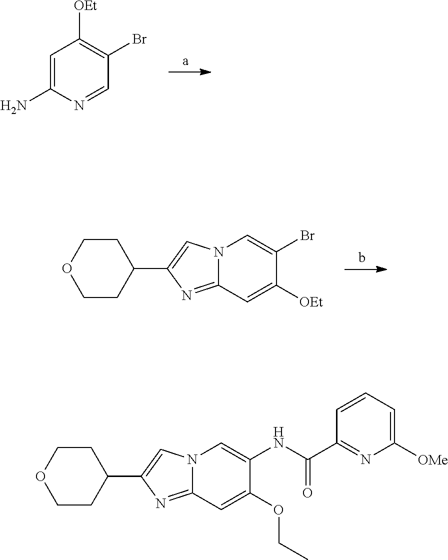 [SMILES: CCOc1cc(N)ncc1Br ; CCOc1cc2nc(C3CCOCC3)cn2cc1Br ; CCOc1cc2nc(C3CCOCC3)cn2cc1NC(=O)c1cccc(OC)n1]

Step a: A vial was charged with 5-bromo-4-ethoxy-pyridin-2-amine (497 mg, 2.29 mmol), NaHCO₃ (577 mg, 6.87 mmol)) and 2-bromo-1-tetrahydropyran-4-yl-ethanone (379 mg, 1.83 mmol). EtOH (9 mL) was added and the mixture was heated at 80° C. overnight. The reaction was cooled to rt, MeOH and silica were added, and the mixture was concentrated. Purification via silica gel chromatography gave 6-bromo-7-ethoxy-2-tetrahydropyran-4-yl-imidazo[1,2-a]pyridine (410 mg, 1.26 mmol, 55% yield). LCMS (ESI) m/z 325.4 (M+H)⁺.

Step b: A 4 mL vial was charged with 6-bromo-7-ethoxy-2-tetrahydropyran-4-yl-imidazo[1,2-a]pyridine (100 mg, 307 μmol), Pd(OAc)₂ (13.8 mg, 61.5 μmol), (5-diphenylphosphanyl-9,9-dimethyl-xanthen-4-yl)-diphenyl-phosphane (71 mg, 123 μmol), Cs₂CO₃ (200 mg, 615 μmol) and 6-methoxypyridine-2-carboxamide (117 mg, 768 μmol). The vial was capped with a septum cap and purged with N₂. Dioxane (3 mL) was added at rt and the vial was sealed with parafilm and heated at 100° C. overnight. The reaction was cooled to rt, filtered through celite, concentrated and purified by TFA-modified mass directed HPLC to obtain N-(7-ethoxy-2-tetrahydropyran-4-yl-imidazo[1,2-a]pyridin-6-yl)-6-methoxy-pyridine-2-carboxamide 2,2,2-trifluoroacetate (37.1 mg, 93.6 μmol, 30.4% yield) as a white solid. LCMS (ESI) m/z 397.5 (M+H)⁺. ¹H NMR (500 MHz, METHANOL-d₄) δ ppm 1.62 (t, J=6.71 Hz, 3H) 1.79-1.90 (m, 2H) 1.95-2.08 (m, 2H) 3.09-3.20 (m, 1H) 3.56-3.70 (m, 2H) 4.02-4.15 (m, 5H) 4.45 (q, J=7.33 Hz, 2H) 7.11 (d, J=9.16 Hz, 1H) 7.24 (s, 1H) 7.83-7.97 (m, 3H) 9.83 (s, 1H).

### Example 134: N-(7-ethoxy-2-((tetrahydrofuran-3-yl)methyl)imidazo[1,2-a]pyridin-6-yl)-6-methoxypicolinamide

Example 134 was prepared in a similar fashion to that described for Example 133, using 1-chloro-3-(tetrahydrofuran-3-yl)propan-2-one in place of 2-bromo-1-tetrahydropyran-4-yl-ethanone. LCMS (ESI) m/z 397.5 (M+H)⁺. ¹H NMR (500 MHz, DMSO-d₆) δ ppm 1.51 (t, J=6.71 Hz, 3H) 1.61 (dd, J=12.51, 7.63 Hz, 1H) 1.98-2.11 (m, 1H) 2.57-2.65 (m, 1H) 2.85 (d, J=7.32 Hz, 2H) 3.68 (q, J=7.73 Hz, 1H) 3.75-3.85 (m, 2H) 4.05 (s, 3H) 4.42 (q, J=7.12 Hz, 2H) 7.21 (d, J=8.55 Hz, 1H) 7.35 (s, 1H) 7.84 (d, J=7.32 Hz, 1H) 8.03 (dd, J=8.55, 7.32 Hz, 1H) 8.15 (s, 1H) 9.79 (s, 1H) 10.49 (s, 1H).

### Example 135 and 136: 6-(difluoromethyl)-N-(7-ethoxy-2-((1r,3r)-3-isopropoxycyclobutyl)imidazo[1,2-a]pyridin-6-yl)picolinamide and 6-(difluoromethyl)-N-(7-ethoxy-2-((1s,3s)-3-isopropoxycyclobutyl)imidazo[1,2-a]pyridin-6-yl)picolinamide

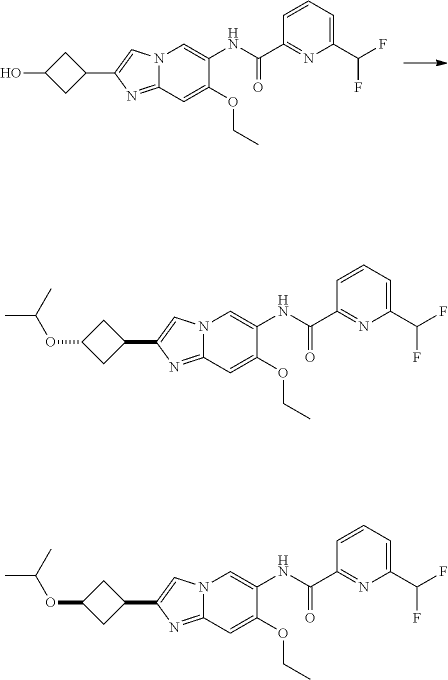

A solution of 6-(difluoromethyl)-N-[7-ethoxy-2-(3-hydroxycyclobutyl)imidazo[1,2-a]pyridin-6-yl]pyridine-2-carboxamide (127 mg, 316 μmol) in acetone (630 μL) was treated with 2-Iodopropane (107 mg, 631 μmol, 63 μL) and Silver(I) oxide (76.8 mg, 331 μmol). The mixture was then heated at 60° C. overnight. The mixture was filtered, concentrated, and purified via HPLC (CHIRALPAK IB 30×250 mm, 5 um, Method: 15% MeOH w/0.1% DEA in CO₂ (flow rate: 100 mL/min, ABPR 120 bar, MBPR 40 psi, column temp 40 deg ° C.) Collection mode was UV collection monitoring at 290 nm) to give 6-(difluoromethyl)-N-[7-ethoxy-2-(3-isopropoxycyclobutyl)imidazo[1,2-a]pyridin-6-yl]pyridine-2-carboxamide (4.5 mg, 10 μmol, 3.2% yield) and 6-(difluoromethyl)-N-[7-ethoxy-2-(3-isopropoxycyclobutyl)imidazo[1,2-a]pyridin-6-yl]pyridine-2-carboxamide (1.7 mg, 3.8 μmol, 1.2% yield).

### Example 135: 6-(difluoromethyl)-N-(7-ethoxy-2-((1r,3r)-3-isopropoxycyclobutyl)imidazo[1,2-a]pyridin-6-yl)picolinamide

LCMS (ESI) m/z 445.3 (M+H)⁺. ¹H NMR (500 MHz, METHANOL-d₄) δ ppm 1.16 (d, J=6.10 Hz, 6H) 1.60 (t, J=6.71 Hz, 3H) 2.02-2.20 (m, 2H) 2.61-2.74 (m, 2H) 3.03-3.12 (m, 1H) 3.64-3.75 (m, 1H) 4.04-4.14 (m, 1H) 4.28 (q, J=6.92 Hz, 2H) 6.69-6.98 (m, 2H) 7.50 (s, 1H) 7.93 (d, J=7.94 Hz, 1H) 8.24 (t, J=7.94 Hz, 1H) 8.37 (d, J=7.94 Hz, 1H) 9.44 (s, 1H)

### Example 136: 6-(difluoromethyl)-N-(7-ethoxy-2-((1s,3s)-3-isopropoxycyclobutyl)imidazo[1,2-a]pyridin-6-yl)picolinamide

LCMS (ESI) m/z 445.3 (M+H)⁺. ¹H NMR (500 MHz, METHANOL-d₄) δ ppm 1.16 (d, J=6.10 Hz, 7H) 1.59 (t, J=7.02 Hz, 3H) 2.39-2.56 (m, 4H) 3.45-3.56 (m, 1H) 3.66 (dt, J=12.21, 6.10 Hz, 1H) 4.27 (q, J=6.71 Hz, 2H) 4.36-4.45 (m, 1H) 6.69-6.96 (m, 2H) 7.55 (s, 1H) 7.92 (d, J=7.94 Hz, 1H) 8.23 (t, J=7.94 Hz, 1H) 8.35 (d, J=7.94 Hz, 1H) 9.43 (s, 1H).

### Example 137: N-(7-ethoxy-2-(1-(tetrahydrofuran-3-yl)ethyl)imidazo[1,2-a]pyridin-6-yl)-6-(trifluoromethyl)picolinamide

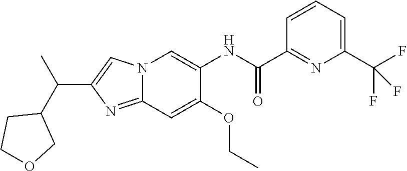 [SMILES: CCOc1cc2nc(C(C)C3CCOC3)cn2cc1NC(=O)c1cccc(C(F)(F)F)n1]

Example 137 was prepared in a similar fashion as Example 82, except 2-(tetrahydrofuran-3-yl)propanoic acid was used in place 2-(1,2,4-triazol-1-yl)acetic acid to provide Example 137 as a mixture of 4 diastereomers. LCMS (ESI) m/z 449.3 (M+H)⁺ Separation of the diastereomers was achieved by chiral SFC separation Column: Chiralpak IC, 250 mm*30 mm, 5 mkm, elution gradient: 40% EtOH w/0.1% DEA in CO₂ (flow rate: 100 mL/min, ABPR 120 bar, MBPR 40 psi, column temp 40 deg C.) to give 4 diastereomers with arbitrarily assigned stereochemistry.

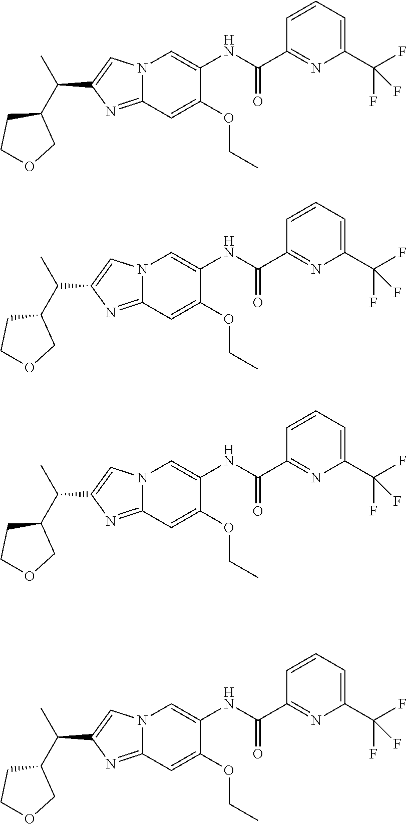

### Example 138: N-(7-ethoxy-2-((R)-1-((S)-tetrahydrofuran-3-yl)ethyl)imidazo[1,2-a]pyridin-6-yl)-6-(trifluoromethyl)picolinamide

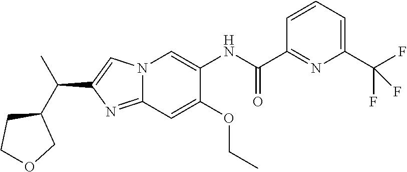 [SMILES: CCOc1cc2nc([C@H](C)[C@@H]3CCOC3)cn2cc1NC(=O)c1cccc(C(F)(F)F)n1]

LCMS (ESI) m/z 449.3 (M+H)⁺; >99% ee; ¹H NMR (400 MHz, CHLOROFORM-d) δ ppm 1.38 (d, J=7.03 Hz, 3H) 1.58 (t, J=6.90 Hz, 3H) 1.66-1.76 (m, 1H) 2.08-2.16 (m, 1H) 2.58-2.66 (m, 1H) 2.76-2.83 (m, 1H) 3.41-3.49 (m, 1H) 3.72-3.79 (m, 2H) 3.89 (td, J=8.28, 3.76 Hz, 1H) 4.17 (q, J=6.94 Hz, 2H) 6.90 (s, 1H) 7.19 (s, 1H) 7.87 (dd, J=7.78, 0.75 Hz, 1H) 8.13 (t, J=7.91 Hz, 1H) 8.43 (d, J=7.78 Hz, 1H) 9.38 (s, 1H) 10.58 (s, 1H).

### Example 139: N-(7-ethoxy-2-((S)-1-((R)-tetrahydrofuran-3-yl)ethyl)imidazo[1,2-a]pyridin-6-yl)-6-(trifluoromethyl)picolinamide

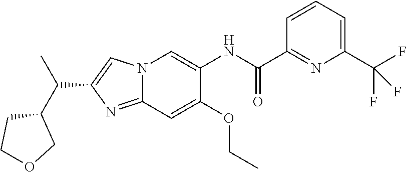

LCMS (ESI) m/z 449.3 (M+H)⁺; 96% ee; ¹H NMR (400 MHz, CHLOROFORM-d) δ ppm 1.38 (d, J=6.78 Hz, 3H) 1.57 (s, 1H) 1.57-1.62 (m, 2H) 1.67-1.76 (m, 1H) 2.07-2.17 (m, 1H) 2.58-2.68 (m, 1H) 2.81 (dq, J=9.29, 6.94 Hz, 1H) 3.43-3.49 (m, 1H) 3.72-3.79 (m, 2H) 3.90 (td, J=8.28, 4.02 Hz, 1H) 4.18 (q, J=7.03 Hz, 2H) 6.92 (s, 1H) 7.20 (s, 1H) 7.88 (dd, J=7.78, 0.75 Hz, 1H) 8.14 (t, J=7.78 Hz, 1H) 8.44 (d, J=7.78 Hz, 1H) 9.39 (s, 1H) 10.59 (s, 1H).

### Example 140: N-(7-ethoxy-2-((S)-1-((S)-tetrahydrofuran-3-yl)ethyl)imidazo[1,2-a]pyridin-6-yl)-6-(trifluoromethyl)picolinamide

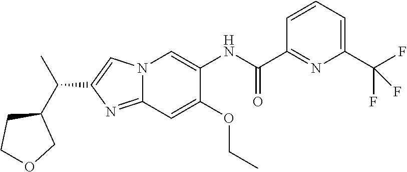 [SMILES: CCOc1cc2nc([C@@H](C)[C@@H]3CCOC3)cn2cc1NC(=O)c1cccc(C(F)(F)F)n1]

LCMS (ESI) m/z 449.3 (M+H)⁺; 96% ee; ¹H NMR (400 MHz, CHLOROFORM-d) δ ppm 1.33 (d, J=7.03 Hz, 3H) 1.56-1.65 (m, 4H) 1.88 (dtd, J=12.17, 7.59, 7.59, 4.27 Hz, 1H) 2.54-2.68 (m, 1H) 2.82 (dq, J=9.22, 6.88 Hz, 1H) 3.56 (t, J=8.03 Hz, 1H) 3.69-3.77 (m, 1H) 3.80-3.86 (m, 1H) 4.00 (t, J=7.91 Hz, 1H) 4.18 (q, J=7.03 Hz, 2H) 6.91-6.97 (m, 1H) 7.22 (s, 1H) 7.88 (dd, J=7.78, 1.00 Hz, 1H) 8.14 (t, J=7.91 Hz, 1H) 8.44 (d, J=7.53 Hz, 1H) 9.40 (s, 1H) 10.60 (s, 1H).

### Example 141: N-(7-ethoxy-2-((R)-1-((R)-tetrahydrofuran-3-yl)ethyl)imidazo[1,2-a]pyridin-6-yl)-6-(trifluoromethyl)picolinamide

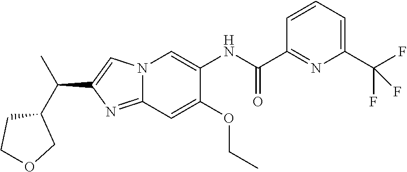 [SMILES: CCOc1cc2nc([C@H](C)[C@H]3CCOC3)cn2cc1NC(=O)c1cccc(C(F)(F)F)n1]

LCMS (ESI) m/z 449.3 (M+H)⁺; >99% ee; ¹H NMR (400 MHz, CHLOROFORM-d) δ ppm 1.34 (d, J=7.03 Hz, 3H) 1.56-1.68 (m, 4H) 1.88 (dtd, J=12.27, 7.61, 7.61, 4.39 Hz, 1H) 2.55-2.68 (m, 1H) 2.82 (dq, J=9.29, 6.94 Hz, 1H) 3.57 (t, J=8.03 Hz, 1H) 3.72 (td, J=8.22, 7.15 Hz, 1H) 3.83 (dt, J=8.28, 4.14 Hz, 1H) 4.00 (t, J=7.91 Hz, 1H) 4.16-4.24 (m, 2H) 6.90-7.01 (m, 1H) 7.22 (s, 1H) 7.85-7.93 (m, 1H) 7.88 (dd, J=7.78, 0.75 Hz, 1H) 7.87-7.87 (m, 1H) 8.14 (t, J=7.78 Hz, 1H) 8.11-8.18 (m, 1H) 8.44 (s, 1H) 8.46 (s, 1H) 9.40 (s, 1H) 10.60 (s, 1H).

### Example 142: N-(7-ethoxy-2-(fluoro(tetrahydrofuran-3-yl)methyl)imidazo[1,2-a]pyridin-6-yl)-6-(trifluoromethyl)picolinamide

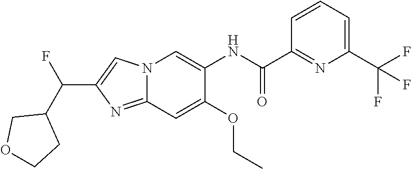 [SMILES: CCOc1cc2nc(C(F)C3CCOC3)cn2cc1NC(=O)c1cccc(C(F)(F)F)n1]

Example 142 was prepared in a similar fashion as Example 82, except 2-fluoro-2-(tetrahydrofuran-3-yl)acetic acid was used in place 2-(1,2,4-triazol-1-yl)acetic acid to provide Example 142 as a mixture of 4 diastereomers. LCMS (ESI) m/z 453.3 (M+H)⁺ Separation of the diastereomers was achieved by chiral SFC separation Column: Chiralpak OX—H, 250 mm*30 mm, 5 mkm, elution gradient: 30% EtOH w/0.1% DEA in CO₂ (flow rate: 100 mL/min, ABPR 120 bar, MBPR 40 psi, column temp 40 deg C.) to provide 4 diastereomers with stereochemistry arbitrarily assigned.

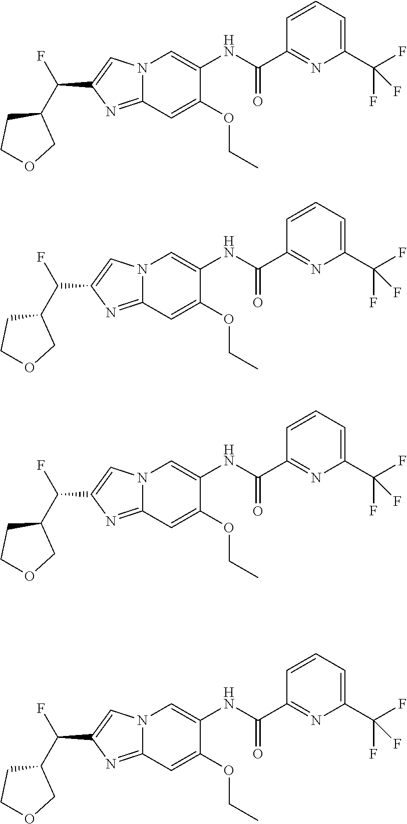

### Example 143: N-(7-ethoxy-2-((R)-fluoro((R)-tetrahydrofuran-3-yl)methyl)imidazo[1,2-a]pyridin-6-yl)-6-(trifluoromethyl)picolinamide

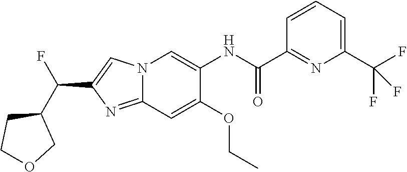 [SMILES: CCOc1cc2nc([C@H](F)[C@@H]3CCOC3)cn2cc1NC(=O)c1cccc(C(F)(F)F)n1]

LCMS (ESI) m/z 453.3 (M+H)⁺; >99% ee; ¹H NMR (400 MHz, METHANOL-d₄) δ ppm 1.58 (t, J=6.90 Hz, 3H) 2.04-2.23 (m, 2H) 3.07-3.15 (m, 1H) 3.51 (dd, J=8.53, 6.53 Hz, 1H) 3.75-3.85 (m, 2H) 3.92 (td, J=8.09, 5.40 Hz, 1H) 4.27 (q, J=6.86 Hz, 2H) 5.36-5.53 (m, 1H) 6.94 (s, 1H) 7.84 (d, J=3.01 Hz, 1H) 8.06 (dd, J=7.91, 0.88 Hz, 1H) 8.30 (t, J=7.91 Hz, 1H) 8.42 (d, J=7.78 Hz, 1H) 9.50 (s, 1H).

### Example 144: N-(7-ethoxy-2-((S)-fluoro((S)-tetrahydrofuran-3-yl)methyl)imidazo[1,2-a]pyridin-6-yl)-6-(trifluoromethyl)picolinamide

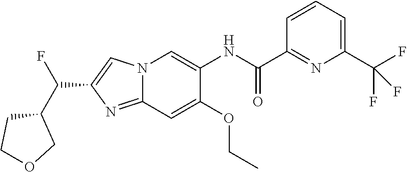

LCMS (ESI) m/z 453.2 (M+H)⁺; 86% ee; ¹H NMR (400 MHz, METHANOL-d₄) δ ppm 1.56 (t, J=6.90 Hz, 3H) 2.04-2.23 (m, 2H) 3.08-3.16 (m, 1H) 3.50 (ddd, J=8.91, 6.27, 0.88 Hz, 1H) 3.73-3.85 (m, 2H) 3.92 (td, J=8.09, 5.40 Hz, 1H) 4.23 (q, J=6.86 Hz, 2H) 5.31-5.48 (m, 1H) 6.88 (s, 1H) 7.77 (d, J=3.01 Hz, 1H) 8.04 (dd, J=7.78, 1.00 Hz, 1H) 8.28 (t, J=7.91 Hz, 1H) 8.40 (d, J=7.78 Hz, 1H) 9.44 (s, 1H).

### Example 145: N-(7-ethoxy-2-((S)-fluoro((R)-tetrahydrofuran-3-yl)methyl)imidazo[1,2-a]pyridin-6-yl)-6-(trifluoromethyl)picolinamide

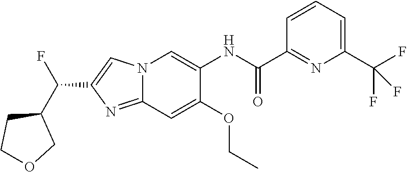 [SMILES: CCOc1cc2nc([C@@H](F)[C@@H]3CCOC3)cn2cc1NC(=O)c1cccc(C(F)(F)F)n1]

LCMS (ESI) m/z 453.2 (M+H)⁺; 96% ee; ¹H NMR (400 MHz, METHANOL-d₄) δ ppm 1.54-1.67 (m, 4H) 1.88-1.98 (m, 1H) 3.03-3.16 (m, 1H) 3.73 (q, J=8.03 Hz, 1H) 3.83-3.89 (m, 1H) 3.92-3.97 (m, 2H) 4.27 (q, J=6.94 Hz, 2H) 5.30-5.47 (m, 1H) 6.94 (s, 1H) 7.83 (d, J=3.26 Hz, 1H) 8.07 (dd, J=7.78, 0.75 Hz, 1H) 8.31 (t, J=7.91 Hz, 1H) 8.45 (d, J=7.78 Hz, 1H) 9.51 (s, 1H).

### Example 146: N-(7-ethoxy-2-((R)-fluoro((S)-tetrahydrofuran-3-yl)methyl)imidazo[1,2-a]pyridin-6-yl)-6-(trifluoromethyl)picolinamide

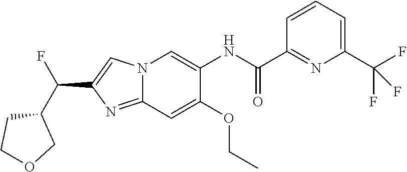 [SMILES: CCOc1cc2nc([C@H](F)[C@H]3CCOC3)cn2cc1NC(=O)c1cccc(C(F)(F)F)n1]

LCMS (ESI) m/z 453.2 (M+H)⁺; 75% ee; ¹H NMR (400 MHz, METHANOL-d₄) δ ppm 1.55-1.65 (m, 4H) 1.89-1.98 (m, 1H) 3.06-3.15 (m, 1H) 3.70-3.77 (m, 1H) 3.87 (td, J=7.72, 5.15 Hz, 1H) 3.94 (d, J=6.27 Hz, 2H) 4.28 (q, J=7.03 Hz, 2H) 5.30-5.47 (m, 1H) 6.94 (s, 1H) 7.83 (d, J=3.01 Hz, 1H) 8.07 (dd, J=7.78, 1.00 Hz, 1H) 8.31 (t, J=7.91 Hz, 1H) 8.45 (d, J=7.78 Hz, 1H) 9.51 (s, 1H).

### Example 147: N-(8-ethoxy-2-(tetrahydro-2H-pyran-4-yl)imidazo[1,2-a]pyrazin-6-yl)-6-methoxypicolinamide

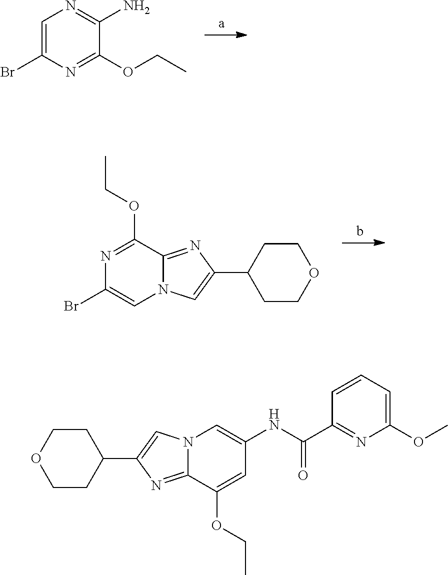 [SMILES: CCOc1nc(Br)cnc1N ; CCOc1nc(Br)cn2cc(C3CCOCC3)nc12 ; CCOc1cc(NC(=O)c2cccc(OC)n2)cn2cc(C3CCOCC3)nc12]

Step a: 6-bromo-8-ethoxy-2-(tetrahydro-2H-pyran-4-yl)imidazo[1,2-a]pyrazine was prepared following the synthetic sequence as described for Intermediate 2 by condensing 5-bromo-3-ethoxypyrazin-2-amine with 2-bromo-1-(tetrahydro-2H-pyran-4-yl)ethan-1-one in place of 2-bromo-1-cyclopropyl-ethanone. LCMS (ESI) m/z 327.0 (M+H)⁺.

Step b: N-(8-ethoxy-2-(tetrahydro-2H-pyran-4-yl)imidazo[1,2-a]pyrazin-6-yl)-6-methoxypicolinamide was prepared following the procedure described for Example 28, except 6-methoxypicolinamide was used in place of 6-(difluoromethyl)picolinamide. LCMS (ESI) m/z 497.2 (M+H)⁺, ¹H NMR (400 MHz, METHANOL-d₄) δ ppm 1.73-1.87 (m, 2H) 1.96-2.03 (m, 2H) 2.93-3.01 (m, 1H) 3.58 (td, J=11.67, 2.01 Hz, 2H) 3.98-4.06 (m, 2H) 5.31 (s, 2H) 7.11 (d, J=1.51 Hz, 1H) 7.31-7.43 (m, 3H) 7.55-7.59 (m, 2H) 7.61 (s, 1H) 8.04 (dd, J=7.78, 0.75 Hz, 1H) 8.28 (t, J=7.91 Hz, 1H) 8.44 (d, J=7.78 Hz, 1H) 8.89 (d, J=1.51 Hz, 1H).

### Example 148: 6-(difluoromethyl)-N-(7-(2-(dimethylamino)ethoxy)-2-(tetrahydro-2H-pyran-4-yl)imidazo[1,2-a]pyridin-6-yl)picolinamide

Example 148 was prepared analogous to Example 105: except 2-bromo-N,N-dimethylethan-1-amine was used in place of iodomethane to yield 6-(difluoromethyl)-N-(7-(2-(dimethylamino)ethoxy)-2-(tetrahydro-2H-pyran-4-yl)imidazo[1,2-a]pyridin-6-yl)picolinamide. LCMS (ESI) m/z: 460.2 (M+H)⁺. ¹H NMR: (400 MHz, MeOD-d₄) δ: 9.80 (s, 1H), 8.42 (d, J=8.0 Hz, 1H), 8.27 (t, J=8.0 Hz, 1H), 7.98 (d, J=8.0 Hz, 1H), 7.94 (s, 1H), 7.48 (s, 1H), 7.00 (t, J=56.0 Hz, 1H), 4.84-4.82 (m, 2H), 4.09-4.05 (m, 2H), 3.88-3.86 (m, 2H), 3.62 (t, J=8.0 Hz, 2H), 3.22-3.16 (m, 1H), 3.10 (s, 6H), 2.07-2.03 (m, 2H), 1.91-1.83 (m, 2H).

### Example 149: N-(2-(1,1-difluoro-3-methoxypropyl)-7-ethoxyimidazo[1,2-a]pyridin-6-yl)-6-(difluoromethyl)picolinamide

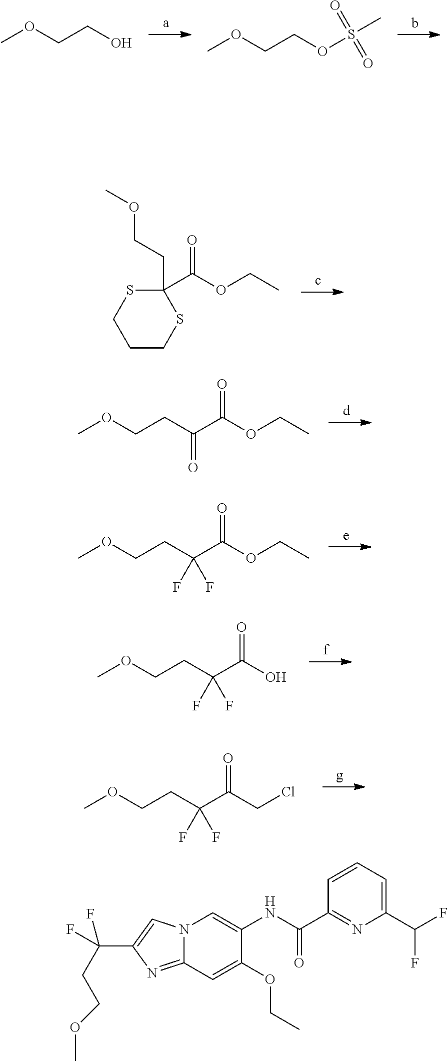

Step a: To a solution of 2-methoxyethan-1-ol (5.00 g, 65.7 mmol) and triethylamine (9.97 g, 98.6 mmol) in DCM (200 mL) was added methanesulfonyl chloride (8.75 g, 76.4 mmol) drop-wise at 0° C. The mixture was stirred at 20° C. for 1 h. The mixture was washed with 1.0N NaOH (100 mL), brine (100 mL), dried over Na₂SO₄, and filtered. The filtrate was concentrated to give 2-methoxyethyl methanesulfonate (6.00 g, 59% yield) as colorless oil, which was used directly in the next step without further purification. ¹H NMR: (500 MHz, CDCl₃) δ: 4.70-4.80 (m, 2H), 3.60-3.70 (m, 2H), 3.40 (s, 3H), 3.06 (s, 3H).

Step b: To a solution of ethyl 1,3-dithiane-2-carboxylate (2.00 g, 10.4 mmol) and 2-methoxyethyl methanesulfonate (3.00 g, 19.5 mmol) in DMF (20 mL) was added NaH (389 mg, 9.73 mmol, 60% weight %) at 0° C. The mixture was stirred at 20° C. for 16 h. The reaction was quenched with saturated aqueous KHSO₄ until pH=5, then it was diluted with water (50 mL) and extracted with EtOAc (50 mL×3). The combined organic layers were washed with brine (100 mL) and dried over Na₂SO₄, filtered. The filtrate was concentrated in vacuo to give the residue, which was purified by column chromatography (PE/EtOAc=10/1 to 3/1) to afford ethyl 2-(2-methoxyethyl)-1,3-dithiane-2-carboxylate (1.00 g, 41% yield) as colorless oil. ¹H NMR: (500 MHz, CDCl₃) δ: 4.26 (q, J=7.5 Hz, 2H), 3.60 (t, J=7.0 Hz, 2H), 3.30 (s, 3H), 3.20-3.30 (m, 2H), 2.60-2.70 (m, 2H), 2.35 (t, J=7.0 Hz, 2H), 2.15-2.20 (m, 1H), 1.80-1.90 (m, 1H), 1.36 (t, J=7.0 Hz, 3H).

Step c: To a solution of ethyl 2-(2-methoxyethyl)-1,3-dithiane-2-carboxylate (1.00 g, 3.99 mmol) in CH₃CN (20 mL) and water (5 mL) was added sodium bromate (1.81 g, 12.0 mmol) and ammonium cerium nitrate (18.7 mg, 0.04 mmol). The mixture was stirred at 20° C. for 1 h. The reaction was quenched with saturated aqueous Na₂SO₃ (30 mL) and washed with DCM (30 mL×3). The combined organic layers were washed with brine (50 mL), dried over Na₂SO₄ and filtered. The filtrate was concentrated in vacuo to give ethyl 4-methoxy-2-oxobutanoate (400 mg, 63% yield) as colorless oil, which was used without further purification. ¹H NMR: (500 MHz, CDCl₃) δ: 4.33 (q, J=7.5 Hz, 2H), 3.73 (t, J=6.0 Hz, 2H), 3.35 (s, 3H), 3.10 (t, J=6.0 Hz, 2H), 1.36 (t, J=7.0 Hz, 3H).

Step d: To a solution of ethyl 4-methoxy-2-oxobutanoate (700 mg, 4.37 mmol) in DCM (20 mL) was added bis(2-methoxyethyl)aminosulfur trifluoride (2.90 g, 13.1 mmol) at 0° C. The mixture was stirred at 20° C. for 16 h. The reaction was quenched with saturated aqueous NaHCO₃ (30 mL) and it was extracted with DCM (30 mL×3). The combined organic layers were washed with brine (50 mL), dried over Na₂SO₄, and filtered. The filtrate was concentrated in vacuo to give a residue, which was purified by silica gel chromatography (PE/EtOAc=10/1 to 3/1) to give ethyl 2,2-difluoro-4-methoxybutanoate (300 mg, 38% yield) as yellow oil. ¹H NMR (500 MHz, CDCl₃) δ: 4.31 (q, J=7.0 Hz, 2H), 3.55 (t, J=6.5 Hz, 2H), 3.29 (s, 3H), 2.40-2.50 (m, 2H), 1.35 (t, J=7.0 Hz, 3H).

Step e: To a solution of ethyl 2,2-difluoro-4-methoxybutanoate (400 mg, 2.20 mmol) in THF (2 mL), MeOH (2 mL) and water (1 mL) was added LiOH (263 mg, 11.0 mmol) at 0° C. The mixture was stirred at 20° C. for 1 h. The reaction was diluted with water (30 mL) and the organic layer was removed and concentrated under reduced pressure to give a residue. HCl (1M) was added until pH=2 and the mixture was extracted with DCM (30 mL×5). The combined organic layers were washed with brine (30 mL), dried over Na₂SO₄, and filtered. The filtrate was concentrated in vacuo to give 2,2-difluoro-4-methoxybutanoic acid (270 mg, 80% yield) as yellow oil, which was used without further purification. ¹H NMR: (500 MHz, CDCl₃) δ: 6.33 (brs, 1H), 3.64 (t, J=8.0 Hz, 2H), 3.36 (s, 3H), 2.40-2.50 (m, 2H).

Step f: To a solution of 2,2-difluoro-4-methoxybutanoic acid (130 mg, 0.844 mmol) in THF (5 mL) was added oxalyl chloride (118 mg, 0.928 mmol) and 1 drop of DMF at 0° C. The mixture was stirred at 0° C. for 1 h before the addition of CH₃CN (5 mL) and trimethylsilyldiazomethane (2 M, 0.845 mL, 1.69 mmol). The mixture was stirred at 20° C. for 2 h, and then to the mixture was added HCl in dioxane (4 M, 0.42 mL, 1.68 mmol). The resulting mixture was stirred at 20° C. for 1 h. The reaction was quenched with saturated aqueous NaHCO₃ (20 mL) and it was extracted with EtOAc (30 mL×3). The combined organic layers were washed with brine (50 mL), dried over Na₂SO₄, and filtered. The filtrate was concentrated in vacuo to give 1-chloro-3,3-difluoro-5-methoxypentan-2-one (100 mg), as yellow oil, which was used in the next step without further purification. ¹H NMR: (500 MHz, CDCl₃) δ: 4.49 (s, 2H), 3.54 (t, J=7.0 Hz, 2H), 3.26 (s, 3H), 2.40-2.50 (m, 2H).

Step g: To a solution of Intermediate 3c: N-(6-amino-4-ethoxypyridin-3-yl)-6-(difluoromethyl)picolinamide (41 mg, 0.14 mmol) and 1-chloro-3,3-difluoro-5-methoxypentan-2-one (50 mg, 0.27 mmol) in EtOH (5 mL) was added NaHCO₃ (23 mg, 0.27 μmol) and KI (2 mg, 0.013 mmol). The mixture was heated at 80° C. for 16 h. The mixture was filtered, and the filtrate was concentrated in vacuo to give the residue, which was purified prep-HPLC to afford N-(2-(1,1-difluoro-3-methoxypropyl)-7-ethoxyimidazo[1,2-a]pyridin-6-yl)-6-(difluoromethyl)picolinamide (12 mg, 20% yield) as an off-white solid. LCMS (ESI) m/z: 441.2 (M+H)⁺. ¹H NMR: (500 MHz, MeOD) δ: 9.55 (s, 1H), 8.36 (d, J=8.0 Hz, 1H), 8.24 (t, J=7.5 Hz, 1H), 8.00-7.90 (m, 2H), 7.00-6.95 (m, 1H), 6.90-6.70 (m, 1H), 4.31 (q, J=7.0 Hz, 2H), 3.56 (t, J=7.0 Hz, 2H), 3.29 (s, 3H), 2.70-2.60 (m, 2H), 1.61 (t, J=7.0 Hz, 3H).

### Example 150: 6-(difluoromethyl)-N-(7-ethoxy-2-(tetrahydrothiophen-3-yl)imidazo[1,2-a]pyridin-6-yl)picolinamide

Example 150 was prepared analogous to Example 82, except tetrahydrothiophene-3-carboxylic acid was used in place of 2-(1,2,4-triazol-1-yl)acetic acid to yield 6-(difluoromethyl)-N-(7-ethoxy-2-(tetrahydrothiophen-3-yl)imidazo[1,2-a]pyridin-6-yl)picolinamide. LCMS (ESI) m/z: 419.1 (M+H)⁺. ¹H NMR: (500 MHz, MeOD) δ: 9.75 (s, 1H), 9.40 (d, J=7.5 Hz, 1H), 8.28 (t, J=8.0 Hz, 1H), 8.00-7.90 (m, 2H), 7.24 (s, 1H), 7.00-6.80 (m, 1H), 4.45 (q, J=7.0 Hz, 2H), 3.80-3.70 (m, 1H), 3.10-3.00 (m, 4H), 2.60-2.50 (m, 1H), 2.40-2.30 (m, 1H) 1.67 (t, J=7.0 Hz, 3H).

### Example 151: 6-(difluoromethyl)-N-(7-ethoxy-2-((tetrahydro-2H-pyran-4-yl)methyl)imidazo[1,2-a]pyridin-6-yl)picolinamide

Example 151 was prepared analogous to Example 82, except 2-(tetrahydro-2H-pyran-4-yl)acetic acid was used in place of 2-(1,2,4-triazol-1-yl)acetic acid to yield 6-(difluoromethyl)-N-(7-ethoxy-2-(tetrahydrothiophen-3-yl)imidazo[1,2-a]pyridin-6-yl)picolinamide. LCMS (ESI) m/z: 431.2 (M+H)⁺. ¹H NMR: (400 MHz, MeOD-d₄) δ: 9.60 (s, 1H), 8.29 (d, J=7.78 Hz, 1H), 8.16 (t, J=7.78 Hz, 1H), 8.09 (br s, 0.5H), 7.86 (d, J=7.78 Hz, 1H), 7.71 (s, 1H), 7.11 (s, 1H), 6.96-6.56 (m, 1H), 4.32 (q, J=7.03 Hz, 2H), 3.85 (br, dd, J=11.29, 3.26 Hz, 2H), 3.32 (br t, J=10.92 Hz, 2H), 2.66 (d, J=7.03 Hz, 2H), 1.83-1.97 (m, 1H), 1.55 (t, J=6.90 Hz, 4H), 1.38-1.15 (m, 3H).

### Example 152: N-(2-(2-cyanopropan-2-yl)-7-ethoxyimidazo[1,2-a]pyridin-6-yl)-6-(difluoromethyl)picolinamide

Example 152 was prepared analogous to Example 82, except 2-cyano-2-methylpropanoic acid was used in place of 2-(1,2,4-triazol-1-yl)acetic acid to yield N-(2-(2-cyanopropan-2-yl)-7-ethoxyimidazo[1,2-a]pyridin-6-yl)-6-(difluoromethyl)picolinamide. LCMS (ESI) m/z: 400.2 (M+H)⁺. ¹H NMR: (500 MHz, MeOD) δ: 9.52 (s, 1H), 8.35 (d, J=8.0 Hz, 1H), 8.24 (t, J=7.5 Hz, 1H), 8.09 (brs, 1H), 7.93 (d, J=7.5 Hz, 1H), 7.78 (s, 1H), 6.70-7.00 (m, 2H), 4.31 (q, J=7.0 Hz, 2H), 1.80 (s, 6H), 1.61 (t, J=7.0 Hz, 3H).

### Example 153: 6-(difluoromethyl)-N-(7-ethoxy-2-(3-methoxybicyclo[1.1.1]pentan-1-yl)imidazo[1,2-a]pyridin-6-yl)picolinamide

Example 153 was prepared in a similar fashion to that described for Example 127, using 3-methoxybicyclo[1.1.1]pentane-1-carboxylic acid in place of 3-fluorobicyclo[1.1.1]pentane-1-carboxylic acid. LCMS (ESI) m/z 429.2 (M+H)⁺. 1H NMR (400 MHz, Methanol-d4) δ 9.42 (s, 1H), 8.34 (d, J=7.8 Hz, 1H), 8.22 (t, J=7.8 Hz, 1H), 7.91 (d, J=7.8 Hz, 1H), 7.50 (s, 1H), 6.95-6.68 (m, 2H), 4.26 (q, J=7.0 Hz, 2H), 3.35 (s, 3H), 2.21 (s, 6H), 1.59 (t, J=7.0 Hz, 3H).

### Example 154: N-(7-ethoxy-2-((tetrahydrofuran-3-yl)methyl)imidazo[1,2-a]pyridin-6-yl)-4-methyloxazole-2-carboxamide

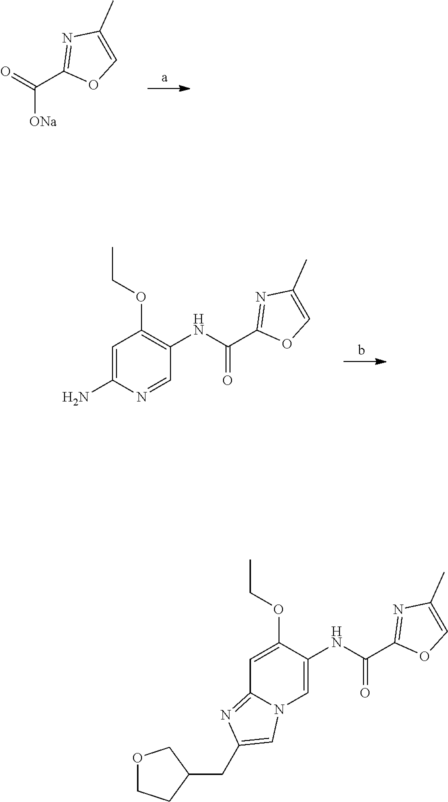

Step a: To a solution of Intermediate 3a, 4-ethoxypyridine-2,5-diamine (200 mg, 1.31 mmol) in DMF (3 mL), HATU (549 mg, 1.44 mmol), DIPEA (500 mg, 3.93 mmol, 690 μL) and sodium 4-methyloxazole-2-carboxylate (167 mg, 1.11 mmol) were added. The mixture was stirred at 30° C. for 3 h. The mixture was diluted with H₂O (50 mL) and extracted with EA (50 mL×4). The combined organic layers were washed with H₂O (50 mL) and brine (50 mL), dried over Na₂SO₄, filtered and concentrated in vacuo. Compound N-(6-amino-4-ethoxy-3-pyridyl)-4-methyl-oxazole-2-carboxamide (363 mg, crude, 82.3% by LCMS) was obtained as brown solid, which was used in the next step without additional purification. LCMS (ESI) m/z 263.2 (M+H)⁺.

Step b: In a sealed tube, a mixture of N-(6-amino-4-ethoxy-3-pyridyl)-4-methyl-oxazole-2-carboxamide (363 mg, 1.14 mmol), 1-chloro-3-tetrahydrofuran-3-yl-propan-2-one (549 mg, 3.42 mmol) and NaHCO₃ (287 mg, 3.42 mmol) in EtOH (10 mL) was heated at 80° C. for 16 h. The mixture was cooled to room temperature, diluted with H₂O (25 mL) and extracted with EtOAc (4×25 mL). The combined organic layers were washed with H₂O (50 mL) and brine (50 mL), dried over Na₂SO₄, filtered and concentrated in vacuo. Purification by HPLC to gave N-[7-ethoxy-2-(tetrahydrofuran-3-ylmethyl)imidazo[1,2-a]pyridin-6-yl]-4-methyl-oxazole-2-carboxamide (21 mg, 5.9% yield). LCMS (ESI) m/z 371.4 (M+H)⁺. ¹H NMR (400 MHz, DMSO-d₆): δ 9.60 (s, 1H), 9.02 (s, 1H), 8.14 (s, 1H), 7.63 (s, 1H), 7.01 (s, 1H), 4.20 (q, J=7.0 Hz, 2H), 3.67-3.82 (m, 2H), 3.50-3.71 (m, 1H), 3.39 (t, J=7.6 Hz, 1H), 2.67 (d, J=7.2 Hz, 2H), 2.52-2.62 (m, 1H), 2.21 (s, 3H), 1.88-2.01 (m, 1H), 1.51-1.64 (m, 1H), 1.39 (t, J=7.0 Hz, 3H).

### Example 155: N-(7-ethoxy-2-((tetrahydrofuran-3-yl)methyl)imidazo[1,2-a]pyridin-6-yl)-1-methyl-1H-pyrazole-3-carboxamide

Example 155 was prepared in a similar fashion to that described for Example 154 using 1-methyl-1H-pyrazole-3-carboxylic acid in place of 1-isopropylpyrazole-3-carboxylic acid. LCMS (ESI) m/z 370.0 (M+H)⁺. ¹H NMR (400 MHz, CDCl₃): δ 9.33 (s, 1H), 9.22 (s, 1H), 7.40 (d, J=2.2 Hz, 1H), 7.18 (s, 1H), 6.86-6.81 (m, 2H), 4.17 (q, J=7.4 Hz, 2H), 3.95 (s, 3H), 3.89 (m, J=7.6 Hz, 2H), 3.75 (q, J=7.7 Hz, 1H), 3.55-3.43 (m, 1H), 2.83-2.62 (m, 2H), 2.11-1.92 (m, 1H), 1.72-1.58 (m 1H), 1.54 (t, J=7.4 Hz, 3H).

### Example 156: 6-(difluoromethyl)-N-(7-ethoxy-2-(1-methyl-2-oxopyrrolidin-3-yl)imidazo[1,2-a]pyridin-6-yl)picolinamide

Example 156, was prepared in a similar fashion to that described for Example 75, using 1-methyl-2-oxopyrrolidine-3-carboxylic acid in place of 1-methyl-2-(400 MHz, DMSO-d₆): δ 10.47 (s, 1H), 9.42 (s, 1H), 8.39-8.22 (m, 2H), 8.00 (dd, J=5.8, 3.1 Hz, 1H), 7.76 (s, 1H), 7.24-6.94 (m, 2H), 4.22 (q, J=6.9 Hz, 2H), 3.66 (t, J=8.4 Hz, 1H), 3.51-3.33 (m, 2H), 2.78 (s, 3H), 2.44-2.17 (m, 2H), 1.49 (t, J=6.9 Hz, 3H). oxopiperidine-4-carboxylic acid. LCMS (ESI) m/z 430.2 (M+H)⁺. ¹H NMR

### Example 157: N-(7-ethoxy-2-((tetrahydrofuran-3-yl)methyl)imidazo[1,2-a]pyridin-6-yl)-1-methyl-1H-imidazole-4-carboxamide

Example 157 was prepared in a similar fashion to that described for Example 154, using 1-methyl-1H-imidazole-4-carboxylic acid in place of 1-isopropylpyrazole-3-carboxylic acid. LCMS (ESI) m/z 370.2 (M+H)⁺. ¹H NMR (400 MHz, Methanol-d4): δ 9.31 (s, 1H), 7.75 (s, 1H), 7.65 (s, 1H), 7.47 (s, 1H), 6.86 (s, 1H), 4.26 (q, J=7.0 Hz, 2H), 3.91-3.82 (m, 2H), 3.79 (s, 3H), 3.79-3.71 (m, 1H), 3.50 (t, J=7.4 Hz, 1H), 2.75 (d, J=7.4 Hz, 2H), 2.69 (m, 1H), 2.14-2.01 (m, 1H), 1.74-1.61 (m, 1H), 1.55 (t, J=7.0 Hz, 3H).

### Example 158: N-(7-ethoxy-2-((tetrahydrofuran-3-yl)methyl)imidazo[1,2-a]pyridin-6-yl)-3-methyl-1,2,4-oxadiazole-5-carboxamide

Example 158 was prepared in a similar fashion to that described for Example 154, using sodium 3-methyl-1,2,4-oxadiazole-5-carboxylate in place of sodium 4-methyloxazole-2-carboxylate. LCMS (ESI) m/z 372.2 (M+H)⁺. ¹H NMR (400 MHz, DMSO-d₆): δ 10.15 (s, 1H), 8.94 (s, 1H), 7.63 (s, 1H), 7.00 (s, 1H), 4.18 (q, J=7.0 Hz, 2H), 3.71-3.80 (m, 2H), 3.63 (q, J=7.4 Hz, 1H), 3.40 (t, J=7.4 Hz, 1H), 2.67 (d, J=7.4 Hz, 2H), 2.55 (m, 1H), 2.50 (s, 3H), 1.89-2.06 (m, 1H), 1.53-1.66 (m, 1H), 1.37 (t, J=7.0 Hz, 3H).

### Example 159: N-(7-ethoxy-2-((tetrahydrofuran-3-yl)methyl)imidazo[1,2-a]pyridin-6-yl)-2-methyloxazole-4-carboxamide

Example 159 was prepared in a similar fashion to that described for Example 154, using 2-methyloxazole-4-carboxylic acid in place of sodium 4-methyloxazole-2-carboxylate. LCMS (ESI) m/z 371.2 (M+H)⁺. ¹H NMR (400 MHz, Methanol-d₄): δ 9.30 (s, 1H), 8.39 (s, 1H), 7.48 (s, 1H), 6.88 (s, 1H), 4.27 (q, J=6.9 Hz, 2H), 3.87 (q, J=7.2, 2H), 3.81-3.72 (m, 1H), 3.56-3.44 (m, 1H), 2.76 (d, J=7.2 Hz, 2H), 2.73-2.62 (m, 1H), 2.52 (s, 3H), 2.12-2.03 (m, 1H), 1.72-1.61 (m, 1H), 1.55 (t, J=6.9 Hz, 3H).

### Example 160: N-(2-(4-cyanotetrahydro-2H-pyran-4-yl)-7-ethoxyimidazo[1,2-a]pyridin-6-yl)-6-(difluoromethyl)picolinamide

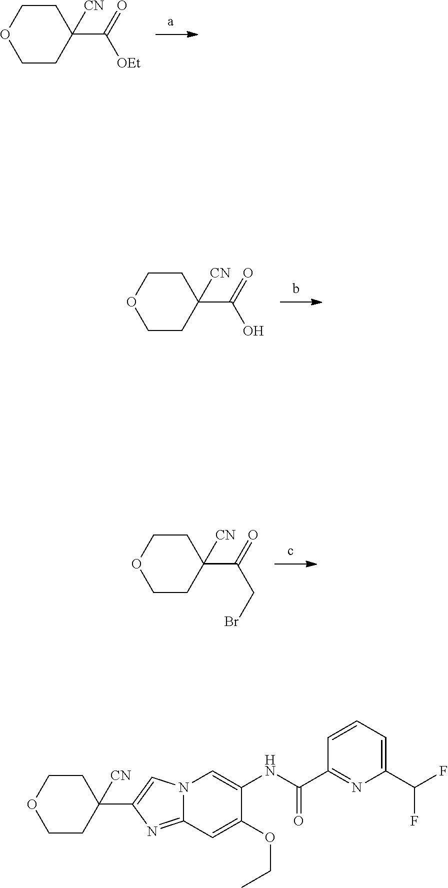 [SMILES: CCOC(=O)C1(C#N)CCOCC1 ; N#CC1(C(=O)O)CCOCC1 ; N#CC1(C(=O)CBr)CCOCC1 ; CCOc1cc2nc(C3(C#N)CCOCC3)cn2cc1NC(=O)c1cccc(C(F)F)n1]

Step a: To a solution of ethyl 4-cyanotetrahydro-2H-pyran-4-carboxylate (2.00 g, 10.9 mmol) in H₂O (10 mL) and CH₃OH (20 mL) was added LiOH (784 mg, 32.8 mmol). The mixture was stirred at 20° C. for 16 h. To the mixture was added 1 M aqueous HCl until pH=3. The mixture was concentrated in vacuo to until aqueous layer remained. The aqueous layer was extracted with EtOAc (30 mL×3). The combined organic layers were washed with brine (50 mL), dried over Na₂SO₄ and filtered. The filtrate was concentrated in vacuo to afford 4-cyanotetrahydro-2H-pyran-4-carboxylic acid (900 mg, 5.80 mmol, 53.1% yield) as white solid, which was used without further purification. ¹H NMR: (400 MHz, CDCl₃) δ: 6.38 (brs, 1H), 4.01-4.05 (m, 2H), 3.82-3.75 (m, 2H), 2.23-2.15 (m, 2H), 2.10-2.06 (m, 2H).

Step b: DIPEA (283 mg, 2.19 mmol, 380 μL) and isobutyl carbonochloridate (264 mg, 1.94 mmol, 250 μL) was added dropwise to a solution of 4-cyanotetrahydro-2H-pyran-4-carboxylic acid in THF (20 mL) at 0° C. The mixture was stirred at 0° C. for 3 h. Then an Et₂O solution of diazomethyl(trimethyl)silane (2 M, 1.3 mL) was added dropwise at 0° C. and the mixture was stirred at 0° C. for 1 h and then 25° C. for 14 h. The mixture was cooled to 0° C., and HBr (652 mg, 3.87 mmol, 440 μL, 48% purity) was added. The reaction mixture stirred at 0° C. for 1 h and was quenched by the addition of saturated aqueous NaHCO₃ until pH=7. The mixture was extracted with EtOAc (20 mL×3) and the combined organic layers were washed with brine (50 mL), dried over Na₂SO₄ and filtered. The filtrate was concentrated in vacuo to give 4-(2-bromoacetyl)tetrahydro-2H-pyran-4-carbonitrile (150 mg, 646 μmol, 50.1% yield) which was used directly in the next step.

Step c: To a solution of Intermediate 3c: N-(6-amino-4-ethoxypyridin-3-yl)-6-(difluoromethyl)picolinamide (50.0 mg, 162 mmol) and 4-(2-bromoacetyl)tetrahydro-2H-pyran-4-carbonitrile (150 mg, 647.1 mmol) in EtOH (5 mL) was added NaHCO₃ (40.9 mg, 486 mmol).

The reaction mixture was stirred at 80° C. for 12 h. The solution was cooled to 20° C. and was concentrated in vacuo to give to give a residue, which was purified by prep-HPLC to afford N-(2-(4-cyanotetrahydro-2H-pyran-4-yl)-7-ethoxyimidazo[1,2-a]pyridin-6-yl)-6-(difluoromethyl)picolinamide (3.0 mg, 6.8 μmol, 4.2% yield) as an off-white solid. LCMS (ESI) m/z: 442.1 (M+H)⁺. ¹H NMR: (400 MHz, MeOD) δ: 9.59 (s, 1H), 8.38 (d, J=7.6 Hz, 1H), 8.25 (t, J=7.6 Hz, 1H), 7.95 (d, J=7.6 Hz, 1H), 7.89 (brs, 1H), 7.03 (s, 1H), 6.99-6.71 (m, 1H), 4.35 (q, J=6.8 Hz, 2H), 4.07-4.03 (m, 2H), 3.88-3.82 (m, 2H), 2.30-2.18 (m, 4H), 1.63 (t, J=6.8 Hz, 3H).

### Example 161: 1-(difluoromethyl)-N-(7-((3-methyloxetan-3-yl)methoxy)-2-(tetrahydro-2H-pyran-4-yl)imidazo[1,2-a]pyridin-6-yl)-1H-pyrazole-4-carboxamide

Example 161 was prepared analogous to example 5, except (3-methyloxetan-3-yl)methanol was used in place of cyclobutylmethanol in Step a, and 2-bromo-1-(tetrahydro-2H-pyran-4-yl)ethan-1-one in place of 2-bromo-1-cyclopropylethan-1-one in Step b, and 1-(difluoromethyl)-1H-pyrazole-4-carboxamide was used in place of 6-(difluoromethyl)picolinamide in Step c to yield 1-(difluoromethyl)-N-(7-((3-methyloxetan-3-yl)methoxy)-2-(tetrahydro-2H-pyran-4-yl)imidazo[1,2-a]pyridin-6-yl)-1H-pyrazole-4-carboxamide. LCMS (ESI) m/z: 462.2 (M+H)⁺. ¹H NMR: (400 MHz, MeOD-d₄) δ: 9.34 (s, 1H), 8.72 (s, 1H), 8.23 (s, 1H), 7.82 (s, 1H), 7.58 (t, J=56.0 Hz, 1H), 7.28 (s, 1H), 4.77 (d, J=4.0 Hz, 2H), 4.61 (d, J=4.0 Hz, 2H), 4.32 (s, 2H), 4.08-4.05 (m, 2H), 3.65-3.56 (m, 2H), 3.17-3.11 (m, 1H), 2.05-2.01 (m, 2H), 1.90-1.81 (m, 2H), 1.42 (s, 3H).

### Example 162: 1-(difluoromethyl)-N-(2-(tetrahydro-2H-pyran-4-yl)imidazo[1,2-a]pyrazin-6-yl)-1H-pyrazole-4-carboxamide

Example 162 was prepared analogous to Example 31, except 2-bromo-1-(tetrahydro-2H-pyran-4-yl)ethan-1-one was used in place of 2-bromo-1-cyclopropylethan-1-one in Step a, and 1-(difluoromethyl)-1H-pyrazole-4-carboxamide was used in place of 6-(difluoromethyl)picolinamide in Step c to yield 1-(difluoromethyl)-N-(2-(tetrahydro-2H-pyran-4-yl)imidazo[1,2-a]pyrazin-6-yl)-1H-pyrazole-4-carboxamide. LCMS (ESI) m/z: 363.1 (M+H)⁺. ¹H NMR: (400 MHz, MeOD-d₄) δ: 9.31 (d, J=4.0 Hz, 1H), 8.78 (s, 1H), 8.76 (s, 1H), 8.27 (s, 1H), 7.93 (s, 1H), 7.59 (t, J=56.0 Hz, 1H), 4.07-4.03 (m, 2H), 3.61 (t, J=12.0 Hz, 2H), 3.14-3.06 (m, 1H), 2.04-2.00 (m, 2H), 1.93-1.83 (m, 2H).

### Example 163: N-(7-((3-methyloxetan-3-yl)methoxy)-2-((tetrahydrofuran-3-yl)methyl)imidazo[1,2-a]pyridin-6-yl)-6-(trifluoromethyl)picolinamide

Example 163 was prepared through the same synthetic sequence as described for the preparation of Example 5, except for substituting (3-methyloxetan-3-yl)methanol in place of cyclobutylmethanol in Step a, and 1-chloro-3-(tetrahydrofuran-3-yl)propan-2-one in place of 2-bromo-1-cyclopropylethan-1-one in Step b, and 6-(trifluoromethyl)picolinamide in place of 6-(difluoromethyl)picolinamide in Step c to yield N-(7-((3-methyloxetan-3-yl)methoxy)-2-((tetrahydrofuran-3-yl)methyl)imidazo[1,2-a]pyridin-6-yl)-6-(trifluoromethyl)picolinamide. LCMS (ESI) m/z 491.2 (M+H)⁺. ¹H NMR (400 MHz, MeOD-d₄) δ ppm 9.48 (s, 1H), 8.45 (d, J=8.0 Hz, 1H), 8.29 (t, J=8.0 Hz, 1H), 8.05 (d, J=8.0 Hz, 1H), 7.51 (s, 1H), 6.99 (s, 1H), 4.67 (d, J=8.0 Hz, 2H), 4.57 (d, J=8.0 Hz, 2H), 4.39 (s, 2H), 3.93-3.86 (m, 2H), 3.78 (q, J=8.0 Hz, 1H), 3.54-3.50 (m, 1H), 2.79-2.77 (m, 2H), 2.75-2.64 (m, 1H), 2.14-2.06 (m, 1H), 1.75-1.66 (m, 1H), 1.57 (s, 3H).

Enantiomers of Example 163 were Separated by Chiral SFC Under the Conditions of:
CHIRALPAK AD-H 30×250 mm, 5 μm
Method: 40% EtOH w/0.1% DEA in CO₂ (flow rate: 100 mL/min, ABPR 120 bar, MBPR 40 psi, column temp 40° C.)

### Example 164: (R)—N-(7-((3-methyloxetan-3-yl)methoxy)-2-((tetrahydrofuran-3-yl)methyl)imidazo[1,2-a]pyridin-6-yl)-6-(trifluoromethyl)picolinamide

Example 164, is peak 1 from the chiral SFC separation and the stereochemistry is arbitrarily assigned. (>99% ee).

LCMS (ESI) m/z 491.2 (M+H)⁺. ¹H NMR (400 MHz, MeOD-d₄) δ ppm 9.48 (s, 1H), 8.45 (d, J=8.0 Hz, 1H), 8.29 (t, J=8.0 Hz, 1H), 8.05 (d, J=8.0 Hz, 1H), 7.51 (s, 1H), 6.99 (s, 1H), 4.67 (d, J=8.0 Hz, 2H), 4.57 (d, J=8.0 Hz, 2H), 4.39 (s, 2H), 3.93-3.86 (m, 2H), 3.78 (q, J=8.0 Hz, 1H), 3.54-3.50 (m, 1H), 2.79-2.77 (m, 2H), 2.75-2.64 (m, 1H), 2.14-2.06 (m, 1H), 1.75-1.66 (m, 1H), 1.57 (s, 3H).

### Example 165: (S)—N-(7-((3-methyloxetan-3-yl)methoxy)-2-((tetrahydrofuran-3-yl)methyl)imidazo[1,2-a]pyridin-6-yl)-6-(trifluoromethyl)picolinamide

Example 165 is peak 2 from the chiral SFC separation and the stereochemistry is arbitrarily assigned. (>99% ee).

LCMS (ESI) m/z 491.2 (M+H)⁺. ¹H NMR (400 MHz, MeOD-d₄) δ ppm 9.48 (s, 1H), 8.45 (d, J=8.0 Hz, 1H), 8.29 (t, J=8.0 Hz, 1H), 8.05 (d, J=8.0 Hz, 1H), 7.51 (s, 1H), 6.99 (s, 1H), 4.67 (d, J=8.0 Hz, 2H), 4.57 (d, J=8.0 Hz, 2H), 4.39 (s, 2H), 3.93-3.86 (m, 2H), 3.78 (q, J=8.0 Hz, 1H), 3.54-3.50 (m, 1H), 2.79-2.77 (m, 2H), 2.75-2.64 (m, 1H), 2.14-2.06 (m, 1H), 1.75-1.66 (m, 1H), 1.57 (s, 3H).

### Example 166: N-(7-((3-methyloxetan-3-yl)methoxy)-2-(tetrahydro-2H-pyran-4-yl)imidazo[1,2-a]pyridin-6-yl)-6-(trifluoromethyl)picolinamide

Example 166 was prepared in a manner analogous to Example 5, except (3-methyloxetan-3-yl)methanol was used in place of cyclobutylmethanol in Step a, and 2-bromo-1-(tetrahydro-2H-pyran-4-yl)ethan-1-one was used in place of 2-bromo-1-cyclopropylethan-1-one in Step b, and 6-(trifluoromethyl)picolinamide was used in place of 6-(difluoromethyl)picolinamide in Step c to yield N-(7-((3-methyloxetan-3-yl)methoxy)-2-(tetrahydro-2H-pyran-4-yl)imidazo[1,2-a]pyridin-6-yl)-6-(trifluoromethyl)picolinamide. LCMS (ESI) m/z: 491.2 (M+H)⁺. ¹H NMR: (400 MHz, MeOD-d₄) δ: 9.86 (s, 1H), 8.54 (d, J=8.0 Hz, 1H), 8.36 (t, J=8.0 Hz, 1H), 8.14 (d, J=8.0 Hz, 1H), 7.93 (s, 1H), 7.41 (s, 1H), 4.68 (d, J=8.0 Hz, 2H), 4.62-4.59 (m, 4H), 4.09-4.07 (m, 2H), 3.62 (t, J=8.0 Hz, 2H), 3.21-3.15 (m, 1H), 2.06-2.03 (m, 2H), 1.91-1.82 (m, 2H), 1.60 (s, 3H).

### Example 167: 6-(difluoromethyl)-N-(7-isopropoxy-2-(tetrahydro-2H-pyran-4-yl)imidazo[1,2-a]pyridin-6-yl)picolinamide

Example 167 was prepared analogous to Example 105, except 2-iodopropane was used in place of iodomethane to yield 6-(difluoromethyl)-N-(7-isopropoxy-2-(tetrahydro-2H-pyran-4-yl)imidazo[1,2-a]pyridin-6-yl)picolinamide. LCMS (ESI) m/z: 431.2 (M+H)⁺. ¹H NMR: (400 MHz, MeOD-d₄) δ: 9.77 (s, 1H), 8.38 (d, J=8.0 Hz, 1H), 8.26 (t, J=8.0 Hz, 1H), 7.96 (d, J=8.0 Hz, 1H), 7.88 (s, 1H), 7.30 (s, 1H), 6.88 (t, J=56.0 Hz, 1H), 5.10-5.01 (m, 1H), 4.08-4.05 (m, 2H), 3.61 (t, J=8.0 Hz, 2H), 3.20-3.12 (m, 1H), 2.06-2.02 (m, 2H), 1.90-1.79 (m, 2H), 1.58 (d, J=8.0 Hz, 6H).

### Example 168: N-(2-(cyclopropyl(hydroxy)methyl)-7-ethoxyimidazo[1,2-a]pyridin-6-yl)-6-(trifluoromethyl)picolinamide

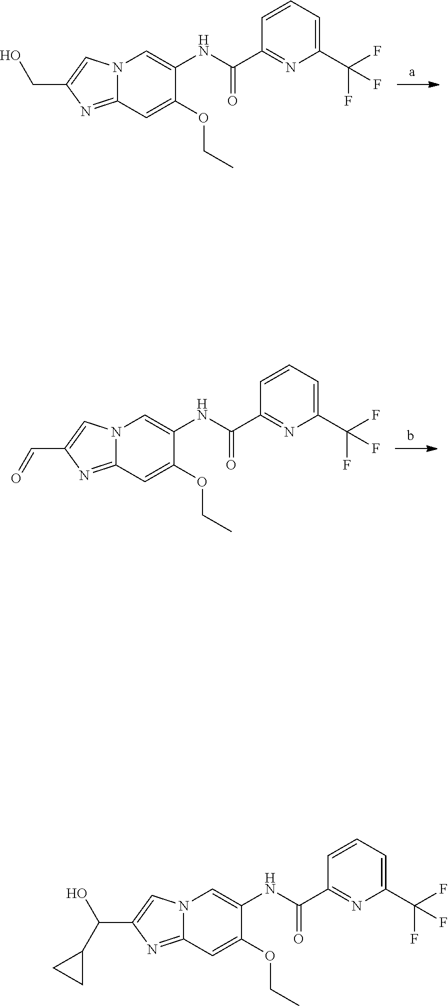 [SMILES: CCOc1cc2nc(CO)cn2cc1NC(=O)c1cccc(C(F)(F)F)n1 ; CCOc1cc2nc(C=O)cn2cc1NC(=O)c1cccc(C(F)(F)F)n1 ; CCOc1cc2nc(C(O)C3CC3)cn2cc1NC(=O)c1cccc(C(F)(F)F)n1]

Step a: To a solution of N-(7-ethoxy-2-(hydroxymethyl)imidazo[1,2-a]pyridin-6-yl)-6-(trifluoromethyl)picolinamide (70 mg, 184 μmol) in DCM (2.4 mL) at rt was added dioxomanganese (160 mg, 1.84 mmol). After stirring for 14 h, the reaction mixture was filtered through celite and concentrated to obtain N-(7-ethoxy-2-formylimidazo[1,2-a]pyridin-6-yl)-6-(trifluoromethyl)picolinamide which was used crude without further purification assuming 100% yield. LCMS (ESI) m/z 379.1 (M+H)+.

Step b: To a solution of N-(7-ethoxy-2-formylimidazo[1,2-a]pyridin-6-yl)-6-(trifluoromethyl)picolinamide (70 mg, 184 μmol) in THF (0.9 mL) at −78° C. mL, was added bromo(cyclopropyl)magnesium (0.5 M THF solution, 0.7 mL). After stirring for 1 h, the reaction mixture was quenched by the addition of saturated aqueous NHCl₄ solution. The layers were separated and the aqueous phase was extracted with EtOAc (3×10 mL), dried over MgSO₄, filtered, concentrated, and purified by TFA-modified mass-directed HPLC on a reverse phase column (5-95% gradient of water to acetonitrile) to obtain N-(2-(cyclopropyl(hydroxy)methyl)-7-ethoxyimidazo[1,2-a]pyridin-6-yl)-6-(trifluoromethyl)picolinamide 2,2,2-trifluoroacetate (1.70 mg, 2% yield over 2 steps). LCMS (ESI) m/z 421.3 (M+H)+, ¹H NMR (400 MHz, METHANOL-d₄) δ ppm 0.50 (s, 1H) 0.59 (s, 1H) 0.71 (d, J=8.03 Hz, 1H) 1.28-1.32 (m, 1H) 1.58-1.62 (m, 1H) 1.64-1.69 (m, 3H) 4.24 (d, J=8.28 Hz, 1H) 4.43-4.49 (m, 2H) 7.25 (s, 1H) 8.03 (s, 1H) 8.13 (d, J=8.03 Hz, 1H) 8.36 (t, J=7.65 Hz, 1H) 8.52 (d, J=8.03 Hz, 1H) 9.81 (s, 1H).

Separation of the diastereomers was achieved via chiral SFC separation Column: Chiralpak AD-H, 250 mm*30 mm, 5 mkm, elution gradient: 40% IPA w/0.1% DEA in CO₂ (flow rate: 100 mL/min, ABPR 120 bar, MBPR 60 psi, column temp 40° C.) to provide 2 enantiomers with stereochemistry arbitrarily assigned.

### Example 169: (S)—N-(2-(cyclopropyl(hydroxy)methyl)-7-ethoxyimidazo[1,2-a]pyridin-6-yl)-6-(trifluoromethyl)picolinamide

Peak 1: LCMS (ESI) m/z 421.3 (M+H)⁺, 100% ee, ¹H NMR (400 MHz, METHANOL-d₄) δ ppm 0.50 (s, 1H) 0.59 (s, 1H) 0.71 (d, J=8.03 Hz, 1H) 1.28-1.32 (m, 1H) 1.58-1.62 (m, 1H) 1.64-1.69 (m, 3H) 4.24 (d, J=8.28 Hz, 1H) 4.43-4.49 (m, 2H) 7.25 (s, 1H) 8.03 (s, 1H) 8.13 (d, J=8.03 Hz, 1H) 8.36 (t, J=7.65 Hz, 1H) 8.52 (d, J=8.03 Hz, 1H) 9.81 (s, 1H), obtained from separation of Example 168, stereochemistry is arbitrarily assigned.

### Example 170: (R)—N-(2-(cyclopropyl(hydroxy)methyl)-7-ethoxyimidazo[1,2-a]pyridin-6-yl)-6-(trifluoromethyl)picolinamide

Peak 2: LCMS (ESI) m/z 421.3 (M+H)⁺, 100% ee, ¹H NMR (400 MHz, METHANOL-d₄) δ ppm 0.50 (s, 1H) 0.59 (s, 1H) 0.71 (d, J=8.03 Hz, 1H) 1.28-1.32 (m, 1H) 1.58-1.62 (m, 1H) 1.64-1.69 (m, 3H) 4.24 (d, J=8.28 Hz, 1H) 4.43-4.49 (m, 2H) 7.25 (s, 1H) 8.03 (s, 1H) 8.13 (d, J=8.03 Hz, 1H) 8.36 (t, J=7.65 Hz, 1H) 8.52 (d, J=8.03 Hz, 1H) 9.81 (s, 1H), obtained from chiral separation of Example 168, stereochemistry is arbitrarily assigned.

### Example 171: 6-(difluoromethyl)-N-(7-ethoxy-2-(5-oxaspiro[2.4]heptan-1-yl)imidazo[1,2-a]pyridin-6-yl)picolinamide

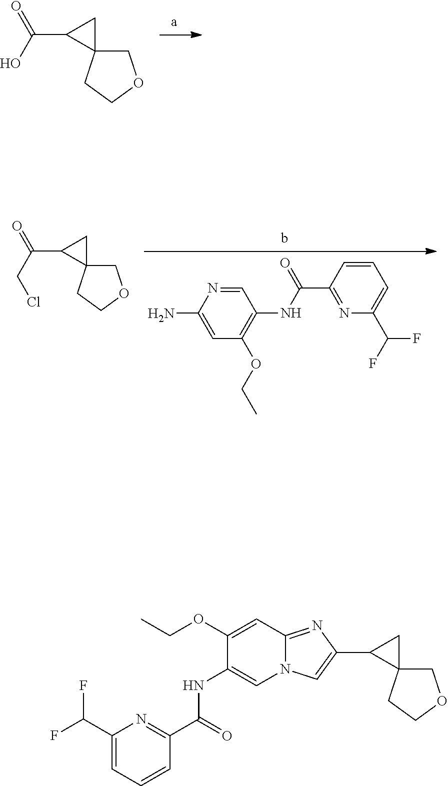 [SMILES: O=C(O)C1CC12CCOC2 ; O=C(CCl)C1CC12CCOC2 ; CCOc1cc(N)ncc1NC(=O)c1cccc(C(F)F)n1 ; CCOc1cc2nc(C3CC34CCOC4)cn2cc1NC(=O)c1cccc(C(F)F)n1]

Step a: A flask was charged with 5-oxaspiro[2.4]heptane-1-carboxylic acid (600 mg, 3.90 mmol), DCM (5 mL), a catalytic amount of DMF and oxalyl chloride (590 mg, 4.29 mmol). The solution was maintained at rt for 20 h, at which time a hexane solution of diazomethyl(trimethyl)silane (2M in hexane, 2.25 mL) was added and the mixture was kept at rt for 20 h. Aqueous hydrochloric acid (15%, 2 eq) was added and the mixture was stirred for 4 h.

The reaction mixture was basified to pH 7 by the addition of saturated aqueous NaHCO₃. The organic phase was separated, concentrated under reduced pressure and distilled at 30 mBar (b.p. 140° C.) to yield 2-chloro-1-(5-oxaspiro[2.4]heptan-1-yl)ethan-1-one (340 mg, 51.1% yield). ¹H NMR (400 MHz, Chloroform-d): δ 4.15 (dd, J=4.8, 2.0 Hz, 2H), 3.59-3.91 (m, 4H), 2.31-2.44 (m, 1H), 1.73-2.08 (m, 2H), 1.27-1.59 (m, 2H).

Step b: 2-chloro-1-(5-oxaspiro[2.4]heptan-1-yl)ethan-1-one (300 mg, 1.37 mmol) and Intermediate 3c, N-(6-amino-4-ethoxypyridin-3-yl)-6-(difluoromethyl)picolinamide, (407 mg, 1.06 mmol) were heated at reflux in propionitrile (20 mL) for 20 h. A saturated aqueous solution of Na₂CO₃ (2 mL) was added and the resulting mixture was stirred for 3 h. The organic phase was separated and concentrated under reduced pressure. The resulting residue was purified by HPLC to yield 6-(difluoromethyl)-N-(7-ethoxy-2-(5-oxaspiro[2.4]heptan-1-yl)imidazo[1,2-a]pyridin-6-yl)picolinamide (23 mg, 5.0% yield) as a mixture of 4 diastereomers. LCMS (ESI) m/z 429.2 (M+H)⁺. 1H NMR (400 MHz, Methanol-d₄): δ 9.35-9.37 (m, 1H), 8.29-8.31 (m, 1H), 8.18-8.21 (m, 1H), 7.8-7.90 (m, 1H), 7.39-7.42 (m, 1H), 6.66-6.94 (m, 2H), 4.19-4.24 (m, 2H), 3.43-3.95 (m, 4H), 2.03-2.25 (m, 2H), 1.66-1.85 (m, 1H), 1.51-1.60 (m, 3H), 1.18-1.31 (m, 2H).

Scheme IV:

### Examples 172 to 198 were Prepared According to Scheme IV, as Follows

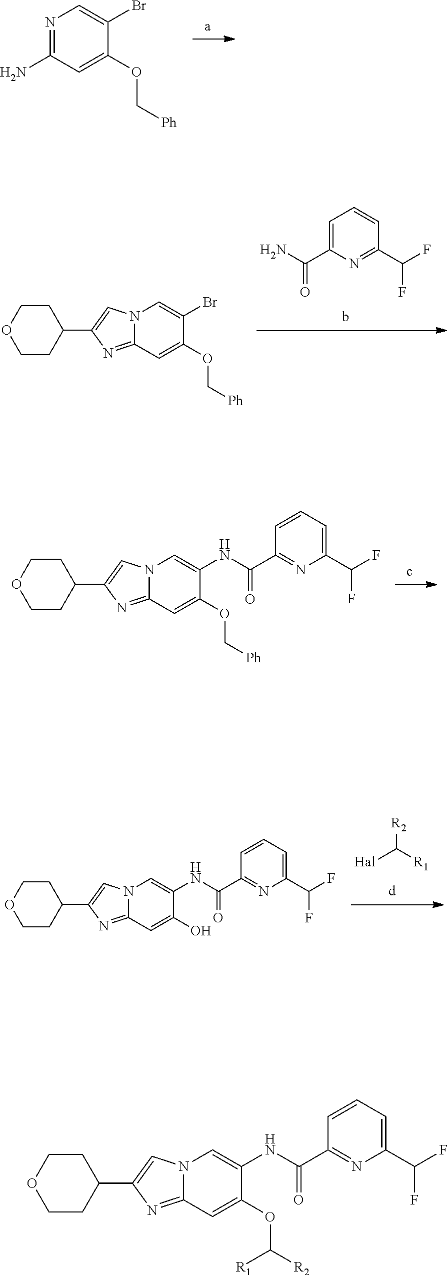

Step a: NaHCO₃ (185.10 mg, 2.21 mmol) was added to a solution of 4-A mg, 1.40 mmol) in EtOH (6 mL) and the reaction heated at 85° C. for 18 h. The cooled reaction was filtered through Celite® washing through with EtOAc and the filtrate evaporated under reduced pressure. The residue was purified by column chromatography via dry load (EtOAc:EtOH in heptanes from 0 to 60%) to give 7-(benzyloxy)-6-bromo-2-(tetrahydro-2H-pyran-4-yl)imidazo[1,2-a]pyridine (233.70 mg, 41.05% yield). Step b: A mixture of 7-(benzyloxy)-6-bromo-2-(tetrahydro-2H-pyran-4-yl)imidazo[1,2-a]pyridine (9.54 g, 24.63 mmol), 6-(difluoromethyl)picolinamide (5.3 g, 30.79 mmol), Cs₂CO₃ (16.05 g, 49.26 mmol) and Xantphos (2.85 g, 4.93 mmol) and Pd(OAc)₂ in dioxane (250 mL) was degassed and the reaction heated under Ar(g) at 100° C. overnight. The reaction was cooled to rt, diluted with EtOAc, filtered and the filtrate concentrated in vacuo. The crude product was purified by silica gel column chromatography (eluting with MeCN/MeOH from 0 to 100%) to give N-(7-(benzyloxy)-2-(tetrahydro-2H-pyran-4-yl)imidazo[1,2-a]pyridin-6-yl)-6-(difluoromethyl)picolinamide, 4.1 g, 34.1% yield, as a yellow solid. LCMS (ESI) m/z 479.2 (M+H)⁺;

Step c: N-(7-(Benzyloxy)-2-(tetrahydro-2H-pyran-4-yl)imidazo[1,2-a]pyridin-6-yl)-6-(difluoromethyl)picolinamide (4.1 g, 8.57 mmol), and 10% Pd/C (400 mg) were dissolved in MeOH (300 mL) and the reaction stirred under 1 psi of H₂ and rt for 24 h. The suspension was filtered through Celite® and the filtrate evaporated under reduced pressure. The solid was washed with DCM (500 mL) and the solvent removed under vacuum to give N-(7-hydroxy-2-(tetrahydro-2H-pyran-4-yl)imidazo[1,2-a]pyridin-6-yl)-6-(difluoromethyl)picolinamide (2.6 g, 6.69 mmol, 78.1% yield) as a tan solid, that was used without purification. LCMS (ESI) m/z 389.2 (M+H)⁺;

Step d: The appropriate alkyl halide (1.2 equiv.) was added to a mixture of 6-(difluoromethyl)-N-(7-hydroxy-2-(tetrahydro-2H-pyran-4-yl)imidazo[1,2-a]pyridin-6-yl)picolinamide (1 equiv.), K₂CO₃ (3 equiv.), KI (0.5 equiv.) and DMF (2 mL) and the reaction stirred at 90° C. for 8 h. The cooled solution was evaporated under reduced pressure at 50° C., the residue was dissolved in DMSO (0.5 mL) and filtered through a paper filter. The solution was purified by prep HPLC eluting with MeOH/H₂O or MeCN/H₂O to obtain the title compound.

Example 172: N-(7-(cyanomethoxy)-2-(tetrahydro-2H-pyran-4-yl)imidazo[1,2-a]pyridin-6-yl)-6-(difluoromethyl)picolinamide: SM: 2-chloroacetonitrile; 5.7 mg, 11.4% as a light brown solid; HPLC gradient: H₂O-MeCN 20-70%; LCMS (ESI) m/z 428.2 (M+H)⁺; ¹H NMR (500 MHz, DMSO-d₆+CCl₄) δ ppm 1.74 (m, 2H), 1.93 (d, J=12.6 Hz, 2H), 2.89 (t, J=13.2 Hz, 1H), 3.47 (t, J=10.9 Hz, 2H), 3.94 (m, 2H), 5.33 (s, 2H), 6.94 (t, J=54.8 Hz, 1H), 7.28 (s, 1H), 7.54 (s, 1H), 7.95 (d, J=7.7 Hz, 1H), 8.28 (t, J=7.7 Hz, 1H), 8.35 (d, J=7.7 Hz, 1H), 9.44 (s, 1H), 10.30 (m, 1H)

Example 173: N-(7-(1-cyanoethoxy)-2-(tetrahydro-2H-pyran-4-yl)imidazo[1,2-a]pyridin-6-yl)-6-(difluoromethyl)picolinamide: SM: 2-bromopropionitrile; 8 mg, 16.0% as brown solid; HPLC gradient: H₂O-MeCN 20-70%; LCMS (ESI) m/z 442.2 (M+H)⁺;

Example 174: 6-(difluoromethyl)-N-(7-isobutoxy-2-(tetrahydro-2H-pyran-4-yl)imidazo[1,2-a]pyridin-6-yl)picolinamide: SM: 1-bromo-2-methylpropane; 4.7 mg, 6.71% yellow solid, HPLC gradient: H₂O-MeOH+NH₃/50-100%; LCMS (ESI) m/z 445.2 (M+H)⁺;

Example 175: N-(7-(cyclopentyloxy)-2-(tetrahydro-2H-pyran-4-yl)imidazo[1,2-a]pyridin-6-yl)-6-(difluoromethyl)picolinamide: SM: bromocyclobutane; 3.6 mg, 7.2% as brown oil, HPLC gradient: H₂O-MeOH, 50-100%, LCMS (ESI) m/z 443.2 (M+H)⁺;

Example 176: N-(7-cyclobutoxy-2-(tetrahydro-2H-pyran-4-yl)imidazo[1,2-a]pyridin-6-yl)-6-(difluoromethyl)picolinamide: SM: bromocyclobutane, 8.3 mg, 16.6% as a yellow solid, HPLC gradient: H₂O/H₂O-MeOH+NH₃/50-100%, LCMS (ESI) m/z 471 (M+H)⁺;

Example 177: 6-(difluoromethyl)-N-(7-(2,2-dimethylcyclobutoxy)-2-(tetrahydro-2H-pyran-4-yl)imidazo[1,2-a]pyridin-6-yl)picolinamide: SM: 2-bromo-1,1-dimethylcyclobutane, 0.3 mg, 16.6% as a yellow solid, HPLC gradient: H₂O/H₂O-MeOH+NH₃/50-100%, LCMS (ESI) m/z 471 (M+H)⁺;

Example 178: 6-(difluoromethyl)-N-(7-((3-methylcyclopentyl)oxy)-2-(tetrahydro-2H-pyran-4-yl)imidazo[1,2-a]pyridin-6-yl)picolinamide: SM: 1-bromo-3-methylcyclopentane, 14.9 mg, 29.8% as a brown solid, HPLC gradient: H₂O-MeOH, 50-100%, LCMS (ESI) m/z 471.2 (M+H)⁺; ¹H NMR (400 MHz, DMSO-d₆+CCl₄) δ ppm 1.12 (d, J=7.0 Hz, 3H), 1.41 (m, 2H), 1.72 (m, 2H), 1.97 (m, 3H), 2.19 (m, 3H), 2.85 (t, 1H), 2.96 (m, 1H), 3.47 (t, J=11.6 Hz, 2H), 3.94 (m, 2H), 5.02 (m, 1H), 6.85 (m, 2H), 7.42 (s, 1H), 7.93 (d, J=7.7 Hz, 1H), 8.26 (t, J=7.9 Hz, 1H), 8.35 (d, J=7.6 Hz, 1H), 9.41 (m, 1H), 10.45 (m, 1H),

Example 179: N-(7-((3-cyanocyclopentyl)oxy)-2-(tetrahydro-2H-pyran-4-yl)imidazo[1,2-a]pyridin-6-yl)-6-(difluoromethyl)picolinamide: SM: 3-bromocyclopentane-1-carbonitrile, 9.4 mg, 18.8% as a brown solid, HPLC gradient H₂O-MeOH, 40-90%, LCMS (ESI) m/z 482.2 (M+H)⁺; ¹H NMR (500 MHz, DMSO-d₆+CCl₄) δ ppm 1.73 (m, 2H), 1.92 (m, 2H), 2.05 (m, 1H), 2.26 (m, 4H), 2.75 (m, 2H), 3.19 (m, 1H), 3.46 (t, J=11.4 Hz, 2H), 3.94 (d, J=11.1 Hz, 2H), 5.18 (m, 1H), 6.97 (m, 2H), 7.46 (s, 1H), 7.93 (t, J=7.1 Hz, 1H), 8.25 (m, 1H), 8.34 (d, J=7.8 Hz, 1H), 9.42 (m, 1H), 10.40 (m, 1H).

Example 180: 6-(difluoromethyl)-N-(7-((3,3-dimethylcyclopentyl)oxy)-2-(tetrahydro-2H-pyran-4-yl)imidazo[1,2-a]pyridin-6-yl)picolinamide: SM: 3-bromo-1,1-dimethylcyclopentane, 15.3 mg, 30.6% as a yellow solid, HPLC gradient H₂O-MeOH+NH₃, 50-100%, LCMS (ESI) m/z 485.2 (M+H)⁺; ¹H NMR (400 MHz, DMSO-d₆+CCl₄) δ ppm 1.10 (s, 3H), 1.17 (s, 3H), 1.56 (m, 1H), 1.73 (m, 3H), 1.83 (m, 1H), 1.92 (m, 2H), 2.07 (m, 2H), 2.35 (m, 1H), 2.85 (t, J=11.2 Hz, 1H), 3.47 (t, J=11.1 Hz, 2H), 3.93 (m, 2H), 5.05 (m, 1H), 6.84 (m, 2H), 7.42 (s, 1H), 7.93 (d, J=7.8 Hz, 1H), 8.26 (t, J=7.8 Hz, 1H), 8.35 (d, J=7.7 Hz, 1H), 9.42 (s, 1H), 10.42 (s, 1H).

Example 181: 6-(difluoromethyl)-N-(7-((1-methyl-2-oxopyrrolidin-3-yl)oxy)-2-(tetrahydro-2H-pyran-4-yl)imidazo[1,2-a]pyridin-6-yl)picolinamide: SM: 3-bromo-1-methylpyrrolidin-2-one, 9.4 mg, 18.8% as a brown solid, HPLC gradient H₂O-MeOH, 40-90%, LCMS (ESI) m/z 486.2 (M+H)⁺; ¹H NMR (500 MHz, DMSO-d₆+CCl₄) δ ppm 1.73 (m, 2H), 1.92 (m, 2H), 2.19 (m, 1H), 2.83 (m, 2H), 2.92 (s, 3H), 3.47 (m, 3H), 3.54 (m, 1H), 3.94 (d, J=10.7 Hz, 2H), 5.10 (t, J=7.2 Hz, 1H), 6.93 (t, J=54.9 Hz, 1H), 7.27 (s, 1H), 7.48 (s, 1H), 7.92 (d, J=7.6 Hz, 1H), 8.26 (t, J=7.8 Hz, 1H), 8.33 (d, J=7.7 Hz, 1H), 9.40 (s, 1H), 10.57 (m, 1H),

Example 182: 6-(difluoromethyl)-N-(7-(oxetan-3-yloxy)-2-(tetrahydro-2H-pyran-4-yl)imidazo[1,2-a]pyridin-6-yl)picolinamide: SM: 3-iodooxetane, 6.8 mg, 13.6% as brown solid, HPLC gradient H₂O-MeOH, 40-90%, LCMS (ESI) m/z 445.2 (M+H)⁺;

Example 183: 6-(difluoromethyl)-N-(2-(tetrahydro-2H-pyran-4-yl)-7-((tetrahydrofuran-3-yl)oxy)imidazo[1,2-a]pyridin-6-yl)picolinamide: SM: 3-bromooxolane, 18 mg, 36% as light brown solid, HPLC gradient H₂O-MeOH, 40-90%, LCMS (ESI) m/z 459.0 (M+H)⁺; 1H NMR (500 MHz, DMSO-d6+CCl₄) δ ppm 1.72 (m, 2H), 1.92 (m, 2H), 2.24 (m, 1H), 2.38 (m, 1H), 2.86 (t, J=11.5 Hz, 1H), 3.46 (t, J=11.4 Hz, 2H), 3.91 (m, 3H), 3.99 (m, 2H), 4.04 (m, 1H), 5.25 (s, 1H), 6.88 (t, J=49.3 Hz, 1H), 7.01 (s, 1H), 7.45 (s, 1H), 7.92 (d, J=7.7 Hz, 1H), 8.26 (t, J=7.8 Hz, 1H), 8.33 (d, J=7.8 Hz, 1H), 9.41 (s, 1H), 10.48 (s, 1H)

Example 184: 6-(difluoromethyl)-N-(7-((5,5-dimethyltetrahydrofuran-3-yl)oxy)-2-(tetrahydro-2H-pyran-4-yl)imidazo[1,2-a]pyridin-6-yl)picolinamide: SM: 4-bromo-2,2-dimethyloxolane, 8.6 mg, 17.2% as brown solid, HPLC gradient H₂O-MeOH, 50-100%, LCMS (ESI) m/z 487.2 (M+H)⁺;

Example 185: 6-(difluoromethyl)-N-(2-(tetrahydro-2H-pyran-4-yl)-7-((tetrahydro-2H-pyran-4-yl)oxy)imidazo[1,2-a]pyridin-6-yl)picolinamide: SM: 4-iodooxane, 7.5 mg, 11.4% as a brown solid, HPLC gradient H₂O-MeOH, 40-90%, LCMS (ESI) m/z 473.2 (M+H)⁺;

Example 186: 6-(difluoromethyl)-N-(7-(((2R*,4S*)-2-methyltetrahydro-2H-pyran-4-yl)oxy)-2-(tetrahydro-2H-pyran-4-yl)imidazo[1,2-a]pyridin-6-yl)picolinamide: SM: trans rac-(2R,4S)-4-bromo-2-methyloxane, 5.2 mg, 10.4%, light brown solid, HPLC gradient H₂O-MeOH, 40-90%, LCMS (ESI) m/z 487.0 (M+H)⁺;

Example 187: 6-(difluoromethyl)-N-(7-(((2R*,4R*,6S*)-2,6-dimethyltetrahydro-2H-pyran-4-yl)oxy)-2-(tetrahydro-2H-pyran-4-yl)imidazo[1,2-a]pyridin-6-yl)picolinamide: SM: rac-(2R,4s,6S)-4-bromo-2,6-dimethyloxane, 5.4 mg, 10.8% as a yellow solid, HPLC gradient:H₂O-MeOH+NH₃/60-100, LCMS (ESI) m/z 501.0 (M+H)⁺;

Example 188: 6-(difluoromethyl)-N-(7-(2-fluoroethoxy)-2-(tetrahydro-2H-pyran-4-yl)imidazo[1,2-a]pyridin-6-yl)picolinamide: SM: 2-fluoroethyl bromide, 9.7 mg, 13.86% as yellow solid, HPLC gradient H₂O-MeOH+NH₃ 40-90%, LCMS (ESI) m/z 435.2 (M+H)⁺; ¹H NMR (400 MHz, CDCl₃) δ ppm 1.84 (m, 2H), 2.01 (m, 2H), 2.96 (t, 1H), 3.55 (t, J=10.9 Hz, 2H), 4.06 (m, 2H), 4.35 (m, 2H), 4.91 (m, 2H), 6.66 (t, J=55.2 Hz, 1H), 6.88 (s, 1H), 7.22 (s, 1H), 7.82 (d, J=7.8 Hz, 1H), 8.08 (t, J=7.8 Hz, 1H), 8.35 (d, J=7.7 Hz, 1H), 9.42 (s, 1H), 10.60 (s, 1H),

Example 189: 6-(difluoromethyl)-N-(7-(3-(difluoromethyl)cyclobutoxy)-2-(tetrahydro-2H-pyran-4-yl)imidazo[1,2-a]pyridin-6-yl)picolinamide: SM: 1-bromo-3-(difluoromethyl)cyclobutene, 13 mg, 18.57% as a brown solid, HPLC gradient H₂O-MeOH+NH₃ 40-90%, LCMS (ESI) m/z 493.2 (M+H)⁺; ¹H NMR (400 MHz, CDCl₃) δ ppm 1.82 (m, 2H), 2.00 (m, 3H), 2.34 (m, 1H), 2.54 (m, 1H), 2.73 (m, 2H), 2.94 (m, 1H), 3.55 (t, J=11.1 Hz, 2H), 4.05 (m, 2H), 4.83 (m, 1H), 5.87 (m, 1H), 6.68 (m, 2H), 7.20 (s, 1H), 7.82 (d, J=7.8 Hz, 1H), 8.08 (t, J=7.7 Hz, 1H), 8.36 (m, 1H), 9.41 (s, 1H), 10.47 (s, 1H).

Example 190: 6-(difluoromethyl)-N-(7-((1R*,3R*)-3-ethoxycyclobutoxy)-2-(tetrahydro-2H-pyran-4-yl)imidazo[1,2-a]pyridin-6-yl)picolinamide: SM: trans-1-bromo-3-ethoxycyclobutane, 11.2 mg, 16% as brown solid, HPLC gradient H₂O-MeOH+NH₃50-100%, LCMS (ESI) m/z 487.2 (M+H); ¹H NMR (400 MHz, CDCl₃) δ ppm 1.20 (t, J=7.0 Hz, 3H), 1.83 (m, 2H), 2.00 (m, 2H), 2.26 (m, 2H), 2.57 (t, J=9.6 Hz, 1H), 2.94 (m, 1H), 3.02 (m, 1H), 3.44 (m, 2H), 3.54 (t, J=11.7 Hz, 2H), 3.82 (m, 1H), 4.05 (m, 2H), 4.39 (m, 1H), 6.68 (m, 2H), 7.20 (s, 1H), 7.82 (d, J=7.8 Hz, 1H), 8.08 (t, J=7.8 Hz, 1H), 8.36 (d, J=7.5 Hz, 1H), 9.41 (s, 1H), 10.46 (m, 1H)

Example 191: N-(7-((3,3-difluorocyclobutyl)methoxy)-2-(tetrahydro-2H-pyran-4-yl)imidazo[1,2-a]pyridin-6-yl)-6-(difluoromethyl)picolinamide: SM: 3-(bromomethyl)-1,1-difluorocyclobutane, 49.1 mg, 70.1% as brown oil, HPLC gradient H₂O-MeOH+NH₃50-100%, LCMS (ESI) m/z 493.2 (M+H)⁺; ¹H NMR (400 MHz, DMSO-d₆+CCl₄) δ ppm 1.71 (m, 2H), 1.92 (m, 2H), 2.44 (m, 2H), 2.84 (m, 4H), 3.46 (t, J=11.6 Hz, 2H), 3.93 (m, 2H), 4.26 (d, J=5.2 Hz, 2H), 6.95 (m, 2H), 7.45 (s, 1H), 7.93 (d, J=7.5 Hz, 1H), 8.26 (t, J=7.8 Hz, 1H), 8.35 (d, J=7.6 Hz, 1H), 9.43 (s, 1H), 10.32 (s, 1H)

Example 192: N-(7-(2-(difluoromethoxy)ethoxy)-2-(tetrahydro-2H-pyran-4-yl)imidazo[1,2-a]pyridin-6-yl)-6-(difluoromethyl)picolinamide: SM: 1-bromo-2-(difluoromethoxy)ethane, 3.5 mg, 5% yield as beige solid, HPLC gradient H₂O-MeOH+NH₃/30-80%, LCMS (ESI) m/z 48.2 (M+H)⁺; ¹H NMR (400 MHz, CDCl₃) δ ppm 1.85 (m, 2H), 2.01 (m, 2H), 2.96 (m, 1H), 3.55 (t, J=14.8, 8.8 Hz, 2H), 4.05 (m, 2H), 4.34 (m, 4H), 6.40 (m, 3H), 6.89 (s, 1H), 7.83 (d, J=7.5 Hz, 1H), 8.09 (t, J=7.9 Hz, 1H), 8.37 (d, J=7.8 Hz, 1H), 9.45 (s, 1H), 10.40 (s, 1H)

Example 193: 6-(difluoromethyl)-N-(7-((tetrahydro-2H-pyran-3-yl)methoxy)-2-(tetrahydro-2H-pyran-4-yl)imidazo[1,2-a]pyridin-6-yl)picolinamide: SM: 3-(bromomethyl)oxane, 2.4 mg, 3.43% as a brown oil, HPLC gradient: H₂O-MeOH+NH₃/30-80%, LCMS (ESI) m/z 487.2 (M+H)⁺;

Example 194: N-(7-((5-oxaspiro[2.4]heptan-1-yl)methoxy)-2-(tetrahydro-2H-pyran-4-yl)imidazo[1,2-a]pyridin-6-yl)-6-(difluoromethyl)picolinamide: SM: 1-(bromomethyl)-5-oxaspiro[2.4]heptane, 24 mg, 34.3% as brown oil, HPLC gradient H₂O-MeOH+NH₃ 30-80%, LCMS (ESI) m/z 499.2 (M+H)⁺; ¹H NMR (400 MHz, DMSO-d₆+CCl₄) δ ppm 0.68 (m, 1H), 1.18 (m, 1H), 1.72 (m, 2H), 1.90 (m, 2H), 2.01 (m, 1H), 2.42 (m, 1H), 2.86 (t, J=14.4 Hz, 1H), 3.19 (d, J=4.3 Hz, 1H), 3.46 (t, J=11.0 Hz, 2H), 3.77 (m, 4H), 3.96 (m, 3H), 4.26 (m, 1H), 6.91 (m, 2H), 7.44 (s, 1H), 7.94 (d, J=7.8 Hz, 1H), 8.27 (t, J=7.8 Hz, 1H), 8.36 (d, J=7.7 Hz, 1H), 9.45 (m, 1H), 10.27 (m, 1H).

Example 195: 6-(difluoromethyl)-N-(2-(tetrahydro-2H-pyran-4-yl)-7-(2-(tetrahydrofuran-3-yl)ethoxy)imidazo[1,2-a]pyridin-6-yl)picolinamide: SM: 3-(2-bromoethyl)oxolane, 25 mg, 35.7% as beige solid, HPLC gradient H₂O-MeOH+NH₃40-90%, LCMS (ESI) m/z 487.2 (M+H)⁺; ¹H NMR (400 MHz, DMSO-d₆+CCl₄) δ ppm 1.68 (m, 3H), 1.92 (m, 2H), 2.04 (m, 2H), 2.13 (m, 1H), 2.54 (m, 1H), 2.86 (m, 1H), 3.46 (m, 3H), 3.67 (q, J=7.8 Hz, 1H), 3.79 (m, 1H), 3.91 (m, 3H), 4.22 (m, 2H), 6.91 (m, 2H), 7.44 (s, 1H), 7.94 (d, J=7.6 Hz, 1H), 8.26 (t, J=7.8 Hz, 1H), 8.36 (d, J=8.0 Hz, 1H), 9.44 (s, 1H), 10.33 (s, 1H)

Example 196: N-(7-((2,2-difluorocyclopropyl)methoxy)-2-(tetrahydro-2H-pyran-4-yl)imidazo[1,2-a]pyridin-6-yl)-6-(difluoromethyl)picolinamide: SM: 2-(bromomethyl)-1,1-difluorocyclopropane, 22.6 mg, 32.3% yield as light brown solid, HPLC gradient H₂O-MeOH+NH₃40-90%, LCMS (ESI) m/z 479.2 (M+H)⁺; ¹H NMR (400 MHz, DMSO-d₆+CCl₄) 8 ppm: 1.56 (m, 1H), 1.74 (m, 3H), 1.92 (m, 2H), 2.33 (m, 1H), 2.87 (m, 1H), 3.46 (t, J=13.5 Hz, 2H), 3.94 (m, 2H), 4.25 (t, J=9.7 Hz, 1H), 4.34 (m, 1H), 6.90 (m, 2H), 7.46 (s, 1H), 7.94 (d, J=7.6 Hz, 1H), 8.35 (d, J=7.7 Hz, 1H), 8.27 (t, J=7.7 Hz, 1H), 9.44 (s, 1H), 10.45 (s, 1H).

Example 197: 6-(difluoromethyl)-N-(7-((3-methyltetrahydrofuran-3-yl)methoxy)-2-(tetrahydro-2H-pyran-4-yl)imidazo[1,2-a]pyridin-6-yl)picolinamide: SM: 3-(bromomethyl)-3-methyloxolane, 18.6 mg, 26.6% yield, as brown solid, HPLC gradient H₂O-MeOH+NH₃ 50-100% LCMS (ESI) m/z 487.2 (M+H)⁺; ¹H NMR (400 MHz, DMSO-d₆+CCl₄) δ ppm: 1.38 (s, 3H), 1.72 (m, 2H), 1.82 (m, 1H), 1.93 (m, 2H), 2.05 (dd, J=13.0, 7.4 Hz, 1H), 2.87 (m, 1H), 3.48 (m, 3H), 3.85 (m, 3H), 3.93 (m, 2H), 4.04 (dd, J=24.3, 9.1 Hz, 2H), 6.86 (m, 2H), 7.45 (s, 1H), 7.94 (d, J=7.6 Hz, 1H), 8.27 (t, J=7.8 Hz, 1H), 8.37 (d, J=7.8 Hz, 1H), 9.46 (s, 1H), 10.36 (m, 1H)

Example 198: 6-(difluoromethyl)-N-(7-((1-(difluoromethyl)cyclobutyl)methoxy)-2-(tetrahydro-2H-pyran-4-yl)imidazo[1,2-a]pyridin-6-yl)picolinamide: SM: 1-(bromomethyl)-1-(difluoromethyl)cyclobutene, 10.9 mg, 15.6%, as beige solid, HPLC gradient H₂O-MeOH+NH₃ 30-80%, LCMS (ESI) m/z 507.2 (M+H)⁺; ¹H NMR (400 MHz, DMSO-d₆) δ ppm: 1.73 (m, 2H), 1.93 (m, 2H), 2.06 (m, 3H), 2.14 (m, 1H), 2.37 (m, 2H), 2.88 (t, J=11.4 Hz, 1H), 3.47 (t, J=12.0 Hz, 2H), 3.94 (m, 2H), 4.34 (s, 2H), 6.16 (t, J=56.7 Hz, 1H), 6.82 (t, J=55.0 Hz, 1H), 7.09 (s, 1H), 7.47 (s, 1H), 7.94 (d, J=7.6 Hz, 1H), 8.26 (t, J=7.8 Hz, 1H), 8.36 (d, J=7.6 Hz, 1H), 9.46 (s, 1H), 10.27 (s, 1H)

Example 199: N-(8-(benzyloxy)-2-(1-methyl-2-oxabicyclo[2.1.1]hexan-4-yl)imidazo[1,2-a]pyrazin-6-yl)-6-(difluoromethyl)picolinamide; was prepared in a manner similar to scheme I or IV, where the Step a condensation reactants are 2-bromo-1-(1-methyl-2-oxabicyclo[2.1.1]hexan-4-yl)ethan-1-one and 3-benzyloxy-5-bromopyrazin-2-amine to yield 8-benzyloxy-6-bromo-2-(1-methyl-2-oxabicyclo[2.1.1]hexan-4-yl)imidazo[1,2-a]pyrazine and Step b proceeds as in scheme 4 to yield Example 199 as a as a pale yellow solid. LCMS (ESI) m/z 492.3 (M+H)⁺; ¹H NMR (400 MHz, METHANOL-d₄) δ ppm: 1.51 (s, 3H), 1.91 (dd, J=1.63, 4.64 Hz, 2H), 2.08-2.25 (m, 2H), 4.05 (s, 2H), 5.68 (s, 2H), 6.78-7.13 (m, 1H), 7.31-7.49 (m, 3H), 7.55-7.70 (m, 2H), 7.87-8.03 (m, 2H), 8.27 (t, J=7.78 Hz, 1H), 8.35-8.50 (m, 1H), 9.07 (s, 1H)

Example 200: 6-(difluoromethyl)-N-(8-hydroxy-2-(1-methyl-2-oxabicyclo[2.1.1]hexan-4-yl)imidazo[1,2-a]pyrazin-6-yl)picolinamide; was prepared by hydrogenation at 1 atmosphere using Pd/C in MeOH to yield Example 200, as a white solid, 10.8 mg. LCMS (ESI) m/z 402.2 (M+H)⁺; ¹H NMR (600 MHz, METHANOL-d₄) δ ppm: 1.51 (s, 3H), 1.82-1.94 (m, 2H), 2.10-2.22 (m, 2H), 3.98-4.09 (m, 2H), 6.76-7.03 (m, 1H), 7.71 (s, 1H), 7.99 (d, J=8.07 Hz, 1H), 8.21-8.31 (m, 2H), 8.38 (d, J=7.34 Hz, 1H).

Example 201: 6-(difluoromethyl)-N-(8-ethoxy-2-(1-methyl-2-oxabicyclo[2.1.1]hexan-4-yl)imidazo[1,2-a]pyrazin-6-yl)picolinamide; was prepared by alkylation of Example 200 as follows,

Ethyl iodide (14.30 mg, 91.68 μmol) was added via a syringe to a sealed vessel containing Example 200: 6-(difluoromethyl)-N-(8-hydroxy-2-(1-methyl-2-oxabicyclo[2.1.1]hexan-4-yl)imidazo[1,2-a]pyrazin-6-yl)picolinamide (34.10 mg, 84.96 μmol) and K₂CO₃ (28.00 mg, 202.59 μmol) in DMF (2 mL) and the reaction was heated at 50° C. for 2 h. The cooled reaction was partitioned between water and EtOAc and the layers separated. The aqueous layer was extracted with EtOAc, the combined organic phases were concentrated in vacuo and the crude purified by column chromatography on silica gel (EtOAc in heptane 50-100%) to give 6-(difluoromethyl)-N-(8-ethoxy-2-(1-methyl-2-oxabicyclo[2.1.1]hexan-4-yl)imidazo[1,2-a]pyrazin-6-yl)picolinamide, 22 mg as a white solid. LCMS (ESI) m/z 430.2 (M+H)⁺; ¹H NMR (400 MHz, CDCl₃) δ ppm: 1.53 (s, 3H), 1.51-1.54 (m, 3H), 1.87-2.15 (m, 4H), 4.09 (s, 2H), 4.69 (q, J=7.03 Hz, 2H), 6.59-6.96 (m, 1H), 7.49 (s, 1H), 7.88 (d, J=7.53 Hz, 1H), 8.12 (t, J=7.78 Hz, 1H), 8.41 (d, J=7.53 Hz, 1H), 8.93 (s, 1H), 9.82 (s, 1H).

### Example 202 to 231 were Prepared According to Scheme V, as Follows

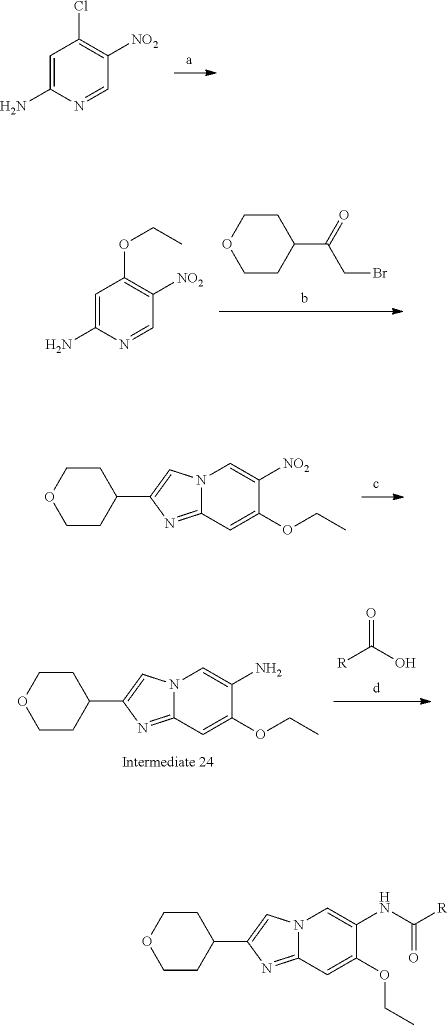

Step a: Sodium ethoxide (39.2 g, 576.18 mmol) in EtOH (200 mL) was added to a suspension of 4-chloro-5-nitropyridin-2-amine (40.0 g, 230.47 mmol) in EtOH (500 mL) and the reaction stirred under reflux for 12 h. The cooled reaction was concentrated in vacuo and the residue suspended in water (250 mL). The crystalline precipitate was filtered off and washed with water to give 4-ethoxy-5-nitropyridin-2-amine (34.3 g, 81.3% yield) as an orange solid. LCMS (ESI) m/z 184.0 (M+H)⁺;

Step b: A mixture of 4-ethoxy-5-nitropyridin-2-amine (20.0 g, 109.19 mmol), 2-bromo-1-(oxan-4-yl)ethan-1-one (45.22 g, 218.38 mmol) and NaHCO₃ (18.34 g, 218.38 mmol) in EtOH (200 mL) was heated at 80° C. for 48 h. The mixture was cooled to rt then diluted with H₂O (100 mL) and extracted with DCM (3×100 mL). The combined organic layers were washed with brine, dried over Na₂SO₄, filtered and evaporated under reduced pressure to give 7-ethoxy-6-nitro-2-(tetrahydro-2H-pyran-4-yl)imidazo[1,2-a]pyridine (24.0 g, 75.5% yield) as brown solid, which was used without further purification. LCMS (ESI) m/z 292.2 (M+H)⁺;

Step c: Iron powder (46 g, 823.89 mmol) was added portion-wise to a solution of 7-ethoxy-6-nitro-2-(tetrahydro-2H-pyran-4-yl)imidazo[1,2-a]pyridine (24.0 g, 82.39 mmol), EtOH (300 mL), H₂O (20 mL), NH₄Cl (100 mg) and 12 N HCl (2 mL) and the reaction stirred under reflux for 5 h. NH₄OH was added to neutralize the reaction, the mixture filtered and the residue washed with hot EtOH. The filtrate was evaporated under reduced pressure to give Intermediate 24: 7-ethoxy-2-(tetrahydro-2H-pyran-4-yl)imidazo[1,2-a]pyridin-6-amine (9.1 g, 34.82 mmol, % yield) as a pale-yellow solid. (ESI) m/z 262.2 (M+H)⁺;

Step d condition 1: was used to prepare examples 202-222 as follows; All the syntheses were performed on a 60 mg-scale (product). Intermediate 24, 7-ethoxy-2-(oxan-4-yl)imidazo[1,2-a]pyridin-6-amine (1.0 equiv.) was added to a mixture of the appropriate carboxylic acid (1.1 equiv.) and EDC (1.21 equiv.) in DMSO (0.8 mL). The reaction mixture was stirred at rt for 24 h. After LCMS showed full conversion, the reaction mixture was filtered and the solution was purified by prep. HPLC (Waters SunFire C18 19*100 5 mkm column; gradient mixture H₂O-MeOH as a mobile phase) to afford the desired product.

Step d condition 2: was used to prepare Examples 223-224 as follows; Intermediate 24, 7-Ethoxy-2-(oxan-4-yl)imidazo[1,2-a]pyridin-6-amine (1.0 equiv.) was added to a mixture of the appropriate carboxylic acid (1.1 equiv.), EDC (1.05 equiv.), HOAt (1.05 equiv.) and DIPEA (2.5 equiv.) in DMF (1 mL) and the reaction stirred at rt for 16 h.

Step d condition 3: was used to prepare Examples 225-231: Methanesulfonyl chloride (1.2 equiv.) was added dropwise to a solution of the appropriate acid (1.2 equiv.) and 1-methyl-1H-imidazole (2.4 equiv.) in DMF (1 mL). The resulting mixture was stirred for 30 min, Intermediate 24, 7-ethoxy-2-(oxan-4-yl)imidazo[1,2-a]pyridin-6-amine (1.0 equiv.) added and the reaction stirred for 1 h at rt, followed by a further 2 h at 100° C. After LCMS showed full conversion, the mixture was evaporated and the residue was dissolved in DMSO (1 mL) and purified by prep. HPLC (Waters SunFire C18 19*100 5 μm column; gradient mixture H₂O-MeOH as a mobile phase) to afford the title compound.

Example 202: N-(7-ethoxy-2-(tetrahydro-2H-pyran-4-yl)imidazo[1,2-a]pyridin-6-yl)-2-ethylthiazole-4-carboxamide: SM: 2-ethyl-1,3-thiazole-4-carboxylic acid, 1.3 mg, 1.19%, HPLC gradient: H₂O-MeOH/50-100%, LCMS (ESI) m/z 401.2 (M+H)⁺;

Example 203: 2-cyclopropyl-N-(7-ethoxy-2-(tetrahydro-2H-pyran-4-yl)imidazo[1,2-a]pyridin-6-yl)thiazole-4-carboxamide: SM: 2-cyclopropyl-1,3-thiazole-4-carboxylic acid, 15.1 mg, 14.5%, HPLC gradient: H₂O-MeOH/50-100%, LCMS (ESI) m/z 413.2 (M+H)⁺; 1H NMR (400 MHz, DMSO-d₆+CCl₄) δ ppm 1.13-1.24 (m, 4H), 1.58 (t, J=6.9 Hz, 3H), 1.68-1.78 (m, 2H), 1.87-1.95 (m, 2H), 2.37-2.45 (m, 1H), 2.80-2.90 (m, 1H), 3.43-3.51 (m, 2H), 3.90-3.97 (m, 2H), 4.22 (q, J=6.9 Hz, 2H), 6.90 (s, 1H), 7.40 (s, 1H), 8.04 (s, 1H), 9.31 (s, 1H), 9.70 (s, 1H)

Example 204: N-(7-ethoxy-2-(tetrahydro-2H-pyran-4-yl)imidazo[1,2-a]pyridin-6-yl)-1-methyl-1H-pyrazole-3-carboxamide: SM: 1-methyl-1H-pyrazole-3-carboxylic acid, 29.9 mg, 24.41%, HPLC gradient:H₂O-MeOH/30-80%, LCMS (ESI) m/z 370.2 (M+H)⁺; 1H NMR (400 MHz, DMSO-d₆+CCl₄) δ ppm 1.57 (t, J=7.0, 7.0 Hz, 3H), 1.66-1.76 (m, 2H), 1.88-1.96 (m, 2H), 2.78-2.91 (m, 1H), 3.42-3.50 (m, 2H), 3.91-3.97 (m, 2H), 4.00 (s, 3H), 4.23-4.32 (m, 2H), 6.70-6.74 (m, 1H), 6.90 (s, 1H), 7.39 (s, 1H), 7.72-7.77 (m, 1H), 9.12 (s, 1H), 9.31 (s, 1H)

Example 205: N-(7-ethoxy-2-(tetrahydro-2H-pyran-4-yl)imidazo[1,2-a]pyridin-6-yl)-1-ethyl-1H-pyrazole-3-carboxamide: SM: 1-ethyl-1H-pyrazole-3-carboxylic acid, 18.6 mg, 19.17%, HPLC gradient:H₂O-MeOH/35-85%, LCMS (ESI) m/z 384.2 (M+H)⁺; ¹H NMR (400 MHz, CDCl₃) δ ppm 1.47-1.58 (m, 6H), 1.75-1.89 (m, 2H), 1.96-2.04 (m, 2H), 2.88-2.99 (m, 1H), 3.48-3.59 (m, 2H), 4.00-4.08 (m, 2H), 4.13-4.18 (m, 2H), 4.18-4.25 (m, 2H), 6.78-6.86 (m, 2H), 7.16 (s, 1H), 7.42 (d, J=2.4 Hz, 1H), 9.29 (s, 1H), 9.34 (s, 1H)

Example 206: N-(7-ethoxy-2-(tetrahydro-2H-pyran-4-yl)imidazo[1,2-a]pyridin-6-yl)-1-isopropyl-1H-pyrazole-3-carboxamide: SM: 1-(propan-2-yl)-1H-pyrazole-3-carboxylic acid, 19.9 mg, 20.22%, HPLC gradient: H₂O-MeOH/35-85%, LCMS (ESI) m/z 398.2 (M+H)⁺; ¹H NMR (400 MHz, CDCl₃) δ ppm 1.49-1.57 (m, 9H), 1.82 (qd, J=12.2, 4.3 Hz, 2H), 1.95-2.02 (m, 2H), 2.88-3.00 (m, 1H), 3.54 (td, J=11.6, 2.1 Hz, 2H), 4.01-4.08 (m, 2H), 4.15 (q, J=6.9, Hz, 2H), 4.45-4.56 (m, 1H), 6.79-6.85 (m, 2H), 7.16 (s, 1H), 7.45 (d, J=2.4 Hz, 1H), 9.33 (s, 1H), 9.36 (s, 1H).

Example 207: 1-(tert-butyl)-N-(7-ethoxy-2-(tetrahydro-2H-pyran-4-yl)imidazo[1,2-a]pyridin-6-yl)-1H-pyrazole-3-carboxamide: SM: 1-(tert-butyl)-1H-pyrazole-3-carboxylic acid, 32.4 mg, 30.99%, HPLC gradient: H₂O-MeOH/40-90%. LCMS (ESI) m/z 412.2 (M+H)⁺; ¹H NMR (400 MHz, CDCl₃) δ ppm 1.54 (t, J=7.0 Hz, 3H), 1.61 (s, 9H), 1.82 (qd, J=12.3, 12.3, 12.1, 4.2 Hz, 2H), 1.98-2.03 (m, 2H), 2.88-2.98 (m, 1H), 3.50-3.58 (m, 2H), 4.01-4.07 (m, 2H), 4.14 (q, J=6.9 Hz, 2H), 6.80-6.85 (m, 2H), 7.16 (s, 1H), 7.55 (d, J=2.4 Hz, 1H), 9.33 (s, 1H), 9.45 (s, 1H)

Example 208: 1-cyclopentyl-N-(7-ethoxy-2-(tetrahydro-2H-pyran-4-yl)imidazo[1,2-a]pyridin-6-yl)-1H-pyrazole-3-carboxamide: SM: 1-cyclopentyl-1H-pyrazole-3-carboxylic acid, 16 mg, 15.9%, HPLC gradient: H₂O-MeOH/40-90%. LCMS (ESI) m/z 424.2 (M+H)⁺; ¹H NMR (400 MHz, CDCl₃) δ ppm 1.54 (t, J=6.9 Hz, 3H), 1.74-1.88 (m, 3H), 1.88-1.96 (m, 2H), 1.97-2.10 (m, 5H), 2.11-2.21 (m, 2H), 2.89-2.99 (m, 1H), 3.48-3.59 (m, 2H), 4.00-4.08 (m, 2H), 4.14 (q, J=6.9 Hz, 2H), 4.65-4.68 (m, 1H), 6.82 (d, J=2.4 Hz, 1H), 6.84 (s, 1H), 7.16 (s, 1H), 7.44 (d, J=2.4 Hz, 1H), 9.34 (s, 1H), 9.36 (s, 1H),

Example 209: 1-(2,2-difluorocyclopropyl)-N-(7-ethoxy-2-(tetrahydro-2H-pyran-4-yl)imidazo[1,2-a]pyridin-6-yl)-1H-pyrazole-3-carboxamide: SM: 1-(2,2-difluorocyclopropyl)-1H-pyrazole-3-carboxylic acid, 16.3 mg, 17.06%, HPLC gradient: H₂O-MeOH/30-80%. LCMS (ESI) m/z 432.2 (M+H); ¹H NMR (400 MHz, CDCl₃) δ ppm 1.54 (t, J=6.9 Hz, 3H), 1.82 (qd, J=12.5, 4.3 Hz, 2H), 1.97-2.04 (m, 2H), 2.09-2.19 (m, 1H), 2.24-2.34 (m, 1H), 2.88-3.00 (m, 1H), 3.49-3.59 (m, 2H), 4.0-4.08 (m, 2H), 4.16 (q, J=6.5, Hz, 3H), 6.84 (s, 1H), 6.90 (d, J=2.3 Hz, 1H), 7.17 (s, 1H), 7.56 (d, J=2.0 Hz, 1H), 9.24 (s, 1H), 9.32 (s, 1H)

Example 210: N-(7-ethoxy-2-(tetrahydro-2H-pyran-4-yl)imidazo[1,2-a]pyridin-6-yl)-5-fluoro-1-methyl-1H-pyrazole-3-carboxamide: SM: 5-fluoro-1-methyl-1H-pyrazole-3-carboxylic acid, 24.2 mg, 26.1%, HPLC gradient: H₂O-MeOH/30-80%. LCMS (ESI) m/z 441.0 (M+H)⁺; 1H NMR (400 MHz, DMSO-d₆+CCl₄) δ ppm 1.56 (t, J=6.9 Hz, 3H), 1.67-1.77 (m, 2H), 1.88-1.95 (m, 2H), 2.82-2.89 (m, 1H), 3.43-3.51 (m, 2H), 3.86 (s, 3H), 3.91-3.96 (m, 2H), 4.26 (q, J=6.9 Hz, 2H), 6.36 (d, J=5.8 Hz, 1H), 6.91 (s, 1H), 7.40 (s, 1H), 9.08 (s, 1H), 9.27 (s, 1H).

Example 211: N-(7-ethoxy-2-(tetrahydro-2H-pyran-4-yl)imidazo[1,2-a]pyridin-6-yl)-3-methoxy-4-methylthiophene-2-carboxamide: SM: 3-methoxy-4-methylthiophene-2-carboxylic acid, 7.4 mg, 8.43%, HPLC gradient: H₂O-MeOH/50-100%. LCMS (ESI) m/z 416.2 (M+H)⁺

Example 212: N-(7-ethoxy-2-(tetrahydro-2H-pyran-4-yl)imidazo[1,2-a]pyridin-6-yl)-2-fluorobenzamide: SM: 2-fluorobenzoic acid, 18.7 mg, 17.08%, HPLC gradient:H₂O-MeOH/40-90%. LCMS (ESI) m/z 384.1 (M+H)⁺; ¹H NMR (400 MHz, CDCl₃) δ ppm 1.53 (t, J=7.0 Hz, 3H), 1.82 (td, J=12.9, 4.4 Hz, 2H), 1.96-2.04 (m, 2H), 2.89-2.99 (m, 1H), 3.49-3.59 (m, 2H), 4.00-4.08 (m, 2H), 4.15 (q, J=6.9 Hz, 2H), 6.84 (s, 1H), 7.13-7.22 (m, 2H), 7.31 (t, J=7.5 Hz, 1H), 7.49-7.56 (m, 1H), 8.18 (td, J=7.9, 1.8 Hz, 1H), 9.32 (d, J=17.0 Hz, 1H), 9.44 (s, 1H)

Example 213: N-(7-ethoxy-2-(tetrahydro-2H-pyran-4-yl)imidazo[1,2-a]pyridin-6-yl)-2-methoxybenzamide: SM: 2-methoxybenzoic acid, 14.2 mg, 14.66%, HPLC gradient:H₂O-MeOH/50-100%. LCMS (ESI) m/z 396.1 (M+H)⁺

Example 214: N-(7-ethoxy-2-(tetrahydro-2H-pyran-4-yl)imidazo[1,2-a]pyridin-6-yl)-2-fluoro-3-methylbenzamide: SM: 2-fluoro-3-methylbenzoic acid, 16.3 mg, 15.8% HPLC gradient:H₂O-MeOH/50-100%. LCMS (ESI) m/z 398.2 (M+H)⁺; 1H NMR (400 MHz, DMSO-d₆+CCl₄) δ ppm 1.55 (t, J=6.9 Hz, 3H), 1.67-1.78 (m, 2H), 1.88-1.96 (m, 2H), 2.36-2.41 (m, 3H), 2.81-2.91 (m, 1H), 3.42-3.51 (m, 2H), 3.90-3.97 (m, 2H), 4.24 (q, J=6.9 Hz, 2H), 6.92 (s, 1H), 7.23 (t, J=7.7 Hz, 1H), 7.41 (s, 1H), 7.43-7.50 (m, 1H), 7.84-7.92 (m, 1H), 9.27 (d, J=15.0 Hz, 1H), 9.38 (s, 1H).

Example 215: N-(7-ethoxy-2-(tetrahydro-2H-pyran-4-yl)imidazo[1,2-a]pyridin-6-yl)-2-fluoro-3-methoxybenzamide: SM: 2-fluoro-3-methoxybenzoic acid, 19.6 mg, 18.48%

HPLC gradient:H₂O-MEOH. LCMS (ESI) m/z 414.2 (M+H)⁺; 1H NMR (400 MHz, DMSO-d₆+CCl₄) δ ppm 1.55 (t, J=7.0 Hz, 3H), 1.68-1.79 (m, 2H), 1.88-1.96 (m, 2H), 2.81-2.90 (m, 1H), 3.46 (t, J=11.1 Hz, 2H), 3.91-3.96 (m, 5H), 4.19-4.27 (m, 2H), 6.91 (s, 1H), 7.23-7.33 (m, 2H), 7.41 (s, 1H), 7.53-7.59 (m, 1H), 9.25 (d, J=14.4 Hz, 1H), 9.36 (s, 1H).

Example 216: N-(7-ethoxy-2-(tetrahydro-2H-pyran-4-yl)imidazo[1,2-a]pyridin-6-yl)picolinamide: SM: pyridine-2-carboxylic acid, 14.7 mg, 14.3%. HPLC gradient: H₂O-MeOH/30-80%. LCMS (ESI) m/z 380.2 (M+H)⁺; 1H NMR (400 MHz, METHANOL-d₄) δ ppm 1.59 (t, J=6.9 Hz, 3H), 1.68-1.78 (m, 2H), 1.97-2.06 (m, 2H), 2.92-3.00 (m, 1H), 3.56-3.64 (m, 2H), 4.01-4.08 (m, 2H), 4.31 (q, J=6.9 Hz, 2H), 6.92 (s, 1H), 7.51 (s, 1H), 7.60-7.66 (m, 1H), 8.01-8.08 (m, 1H), 8.24 (d, J=7.8 Hz, 1H), 9.49 (s, 1H)

Example 217: N-(7-ethoxy-2-(tetrahydro-2H-pyran-4-yl)imidazo[1,2-a]pyridin-6-yl)-6-ethylpicolinamide: SM: 6-ethylpyridine-2-carboxylic acid, 22.9 mg, 20.98%. HPLC gradient: H₂O-MeOH/50-100%. LCMS (ESI) m/z 395.2 (M+H)⁺; 1H NMR (400 MHz, DMSO-d₆+CCl₄) 6 ppm 1.41 (t, J=7.5 Hz, 3H), 1.60 (t, J=6.9 Hz, 3H), 1.67-1.79 (m, 2H), 1.88-1.96 (m, 2H), 2.84-2.95 (m, 3H), 3.41-3.52 (m, 2H), 3.90-3.97 (m, 2H), 4.24 (q, J=7.0 Hz, 2H), 6.92 (s, 1H), 7.42 (s, 1H), 7.45 (d, J=7.6 Hz, 1H), 7.91 (t, J=7.7 Hz, 1H), 8.01 (d, J=7.6 Hz, 1H), 9.44 (s, 1H), 10.61 (s, 1H).

Example 218: N-(7-ethoxy-2-(tetrahydro-2H-pyran-4-yl)imidazo[1,2-a]pyridin-6-yl)-6-isopropylpicolinamide: SM: 6-(propan-2-yl)pyridine-2-carboxylic acid, 20.1 mg, 22.35% HPLC gradient:H₂O-MeOH/50-100%. LCMS (ESI) m/z 409.2 (M+H)⁺; 1H NMR (400 MHz, DMSO-d₆+CCl₄) δ ppm 1.38 (d, J=6.8 Hz, 6H), 1.60 (t, J=7.0 Hz, 3H), 1.69-1.79 (m, 2H), 1.89-1.96 (m, 2H), 2.80-2.91 (m, 1H), 3.12-3.19 (m, 1H), 3.42-3.52 (m, 2H), 3.90-3.98 (m, 2H), 4.24 (q, J=6.8 Hz, 2H), 6.93 (s, 1H), 7.42 (s, 1H), 7.46 (d, J=7.7 Hz, 1H), 7.92 (t, J=7.7 Hz, 1H), 8.01 (d, J=7.5 Hz, 1H), 9.45 (s, 1H), 10.62 (s, 1H).

Example 219: N-(7-ethoxy-2-(tetrahydro-2H-pyran-4-yl)imidazo[1,2-a]pyridin-6-yl)-5-fluoropicolinamide: SM: 5-fluoropyridine-2-carboxylic acid, 4.6 mg, 4.32%. HPLC gradient: H₂O-MeOH/30-80%. LCMS (ESI) m/z 385.2 (M+H)⁺;

Example 220: N-(7-ethoxy-2-(tetrahydro-2H-pyran-4-yl)imidazo[1,2-a]pyridin-6-yl)-6-isopropoxypicolinamide: SM: 6-(propan-2-yloxy)pyridine-2-carboxylic acid, 13.8 mg, 12.7% HPLC gradient:H₂O-MEOH. LCMS (ESI) m/z 425.2 (M+H)⁺; 1H NMR (400 MHz, DMSO-d₆+CCl₄) δ ppm 1.42 (d, J=6.1 Hz, 6H), 1.54 (t, J=6.9 Hz, 3H), 1.71-1.73 (m, 2H), 1.88-1.95 (m, 2H), 2.85 (tt, J=11.3, 3.8, Hz, 1H), 3.42-3.51 (m, 2H), 3.90-3.97 (m, 2H), 4.25 (q, J=6.9 Hz, 2H), 5.44-5.52 (m, 1H), 6.92 (d, J=8.2 Hz, 1H), 6.94 (s, 1H), 7.42 (s, 1H), 7.76 (d, J=7.2 Hz, 1H), 7.86 (t, J=7.7 Hz, 1H), 9.47 (s, 1H), 10.15 (s, 1H)

Example 221: N-(7-ethoxy-2-(tetrahydro-2H-pyran-4-yl)imidazo[1,2-a]pyridin-6-yl)-2,3-dihydrothieno[3,4-b][1,4]dioxine-5-carboxamide: SM: 2H,3H-thieno[3,4-b][1,4]dioxine-5-carboxylic acid, 14.8 mg, 16.8%. HPLC gradient:H₂O-MeOH/40-90%. LCMS (ESI) m/z 430.2 (M+H)⁺;

Example 222: N-(7-ethoxy-2-(tetrahydro-2H-pyran-4-yl)imidazo[1,2-a]pyridin-6-yl)-5,6,7,8-tetrahydroindolizine-1-carboxamide: SM: 5,6,7,8-tetrahydroindolizine-1-carboxylic acid, 12.7 mg, 11.77%. HPLC gradient:H₂O-MeOH/50-100%. LCMS (ESI) m/z 395.2 (M+H)⁺; 1H NMR (400 MHz, DMSO-d₆+CCl₄) δ ppm 1.54 (t, J=6.9 Hz, 3H), 1.70-1.72 (m, 2H), 1.84-1.89 (m, 2H), 1.89-1.94 (m, 2H), 1.94-2.02 (m, 2H), 2.84 (tt, J=11.2, 3.9 Hz, 1H), 3.09 (t, J=6.4 Hz, 2H), 3.45 (td, J=11.5, 2.2 Hz, 2H), 3.89-3.96 (m, 2H), 3.98 (t, J=5.9 Hz, 2H), 4.21 (q, J=6.9 Hz, 2H), 6.33 (d, J=3.1 Hz, 1H), 6.55 (d, J=3.0 Hz, 1H), 6.85 (s, 1H), 7.34 (s, 1H), 8.02 (s, 1H), 9.22 (s, 1H).

Examples 223: N-(7-ethoxy-2-(tetrahydro-2H-pyran-4-yl)imidazo[1,2-a]pyridin-6-yl)-2-methylthiazole-4-carboxamide: SM: 2-methylthiazole-4-carboxylic acid, 8.5 mg, 17%. HPLC gradient: H₂O-MeOH/40-90%. LCMS (ESI) m/z 387.0 (M+H)⁺;

Examples 224: N-(7-ethoxy-2-(tetrahydro-2H-pyran-4-yl)imidazo[1,2-a]pyridin-6-yl)-6-methylpicolinamide: SM: 6-methylpyridine-2-carboxylic acid, 11.9 mg, 23.8%. HPLC gradient:H₂O-MeOH/40-90%. LCMS (ESI) m/z 381.0 (M+H)⁺;

Example 225: N-(7-ethoxy-2-(tetrahydro-2H-pyran-4-yl)imidazo[1,2-a]pyridin-6-yl)-2,3-dihydro-1H-pyrrolizine-7-carboxamide: SM: 2,3-dihydro-1H-pyrrolizine-7-carboxylic acid, 5 mg, 5%. HPLC gradient:H₂O-MeOH/65-90%. LCMS (ESI) m/z 395.2 (M+H)⁺;

Example 226: N-(7-ethoxy-2-(tetrahydro-2H-pyran-4-yl)imidazo[1,2-a]pyridin-6-yl)-2-methoxypyrimidine-4-carboxamide: SM: 2-methoxypyrimidine-4-carboxylic acid, 5.5 mg, 4.92%. HPLC gradient: H₂O-MeOH/50-100%. LCMS (ESI) m/z 398.2 (M+H)⁺;

Example 227: 6-(1,1-difluoroethyl)-N-(7-ethoxy-2-(tetrahydro-2H-pyran-4-yl)imidazo[1,2-a]pyridin-6-yl)picolinamide: SM: 6-(1,1-difluoroethyl)picolinic acid, 10.2 mg, 10.64%. HPLC gradient:H₂O-MeOH/55-100%. LCMS (ESI) m/z 431.0 (M+H)⁺;

Example 228: 3-(difluoromethyl)-N-(7-ethoxy-2-(tetrahydro-2H-pyran-4-yl)imidazo[1,2-a]pyridin-6-yl)benzamide: SM: 3-(difluoromethyl)benzoic acid, 19.6 mg, 18.74%. HPLC gradient: H₂O-MeOH/50-90%. LCMS (ESI) m/z 416.2 (M+H)⁺; 1H NMR (400 MHz, DMSO-d₆) δ ppm 1.52 (t, J=6.9 Hz, 3H), 1.74 (qd, J=12.1, 4.3 Hz, 2H), 1.90-2.03 (m, 2H), 2.92-2.95 (m, 1H), 3.48 (td, J=11.6, 2.1 Hz, 2H), 3.92-3.99 (m, 2H), 4.26 (q, J=6.9 Hz, 2H), 6.85-7.10 (m, 2H), 7.58 (s, 1H), 7.65 (t, J=7.7 Hz, 1H), 7.77 (d, J=7.6 Hz, 1H), 8.10 (d, J=7.8 Hz, 1H), 8.15 (s, 1H), 9.08 (s, 1H), 9.47 (s, 1H).

Example 229: N-(7-ethoxy-2-(tetrahydro-2H-pyran-4-yl)imidazo[1,2-a]pyridin-6-yl)-3-methylbenzamide: SM: 3-methylbenzoic acid, 20.4 mg, 18.3%. HPLC gradient:H₂O-MeCN/35-60%. LCMS (ESI) m/z 380.2 (M+H)⁺; ¹H NMR (400 MHz, DMSO-d₆+CCl₄) δ ppm 1.52 (t, J=6.9 Hz, 3H), 1.67-1.79 (m, 2H), 1.88-1.96 (m, 2H), 2.46 (s, 3H), 2.80-2.91 (m, 1H), 3.42-3.52 (m, 2H), 3.90-3.98 (m, 2H), 4.22 (q, J=6.8 Hz, 2H), 6.89 (s, 1H), 7.35-7.39 (m, 2H), 7.41 (s, 1H), 7.69 (d, J=6.7 Hz, 1H), 7.73 (s, 1H), 8.95 (s, 1H), 9.08 (s, 1H)

Example 230: 1-(difluoromethyl)-N-(7-ethoxy-2-(tetrahydro-2H-pyran-4-yl)imidazo[1,2-a]pyridin-6-yl)-1H-pyrazole-3-carboxamide: SM: 1-(difluoromethyl)-1H-pyrazole-3-carboxylic acid, 20.4 mg, 19.19%. HPLC gradient:H₂O-MeOH/50-100%. LCMS (ESI) m/z 380.2 (M+H)⁺; ¹H NMR (400 MHz, DMSO-d₆+CCl₄) δ ppm 1.56 (t, J=6.9 Hz, 3H), 1.67-1.75 (m, 2H), 1.90-1.96 (m, 2H), 2.81-2.90 (m, 1H), 3.44-3.50 (m, 2H), 3.91-3.96 (m, 2H), 4.26 (q, J=7.1 Hz, 2H), 6.92 (s, 1H), 6.95 (d, J=2.5 Hz, 1H), 7.42 (s, 1H), 7.78 (t, J=58.9 Hz, 1H), 8.27 (d, J=2.6 Hz, 1H), 9.24 (s, 1H), 9.28 (s, 1H)

Example 231: N-(7-ethoxy-2-(tetrahydro-2H-pyran-4-yl)imidazo[1,2-a]pyridin-6-yl)-2-(trifluoromethyl)thiazole-4-carboxamide: SM: 2-(trifluoromethyl)-1,3-thiazole-4-carboxylic acid, 27.4 mg, 24.5%. HPLC gradient:H₂O-MeOH/45-80%. LCMS (ESI) m/z 441.0 (M+H)⁺; ¹H NMR (400 MHz, DMSO-d₆+CCl₄) δ ppm 1.56 (t, J=6.9 Hz, 3H), 1.67-1.78 (m, 2H), 1.89-1.96 (m, 2H), 2.83-2.90 (m, 1H), 3.43-3.51 (m, 2H), 3.91-3.97 (m, 2H), 4.25 (q, J=6.9 Hz, 2H), 6.96 (s, 1H), 7.46 (s, 1H), 8.82 (s, 1H), 9.32 (s, 1H), 9.65 (s, 1H)

### Intermediate 18: 7-isopropoxy-2-(tetrahydro-2H-pyran-4-yl)imidazo[1,2-a]pyridin-6-amine

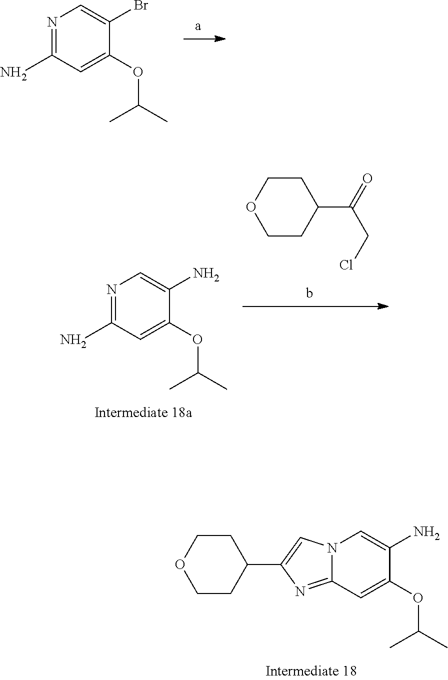 [SMILES: CC(C)Oc1cc(N)ncc1Br ; O=C(CCl)C1CCOCC1 ; CC(C)Oc1cc(N)ncc1N ; CC(C)Oc1cc2nc(C3CCOCC3)cn2cc1N]

Step a: A mixture of 5-bromo-4-isopropoxypyridine-2-amine (13.50 g, 58.42 mmol), copper sulfate (93.24 mg, 584.20 μmol) and 25% aq. NH₄OH (160 mL) was heated in a sealed vessel at 140° C. overnight. The cooled reaction was evaporated under reduced pressure, saturated aqueous K₂CO₃ (150 mL) was added and the mixture extracted with DCM (3×120 mL). The combined organic extracts were dried over Na₂SO₄, filtered and concentrated in vacuo to give Intermediate 18a 4-isopropoxypyridine-2,5-diamine (8.80 g, 88.29% yield) as a dark-brown solid. LCMS (ESI) m/z 168.2 (M+H)⁺

Step b: A mixture of 4-isopropoxypyridine-2,5-diamine (3.03 g, 18.12 mmol), 2-chloro-1-(tetrahydro-2H-pyran-4-yl)ethan-1-one (3.24 g, 19.93 mmol) and NaHCO₃ (4.57 g, 54.36 mmol) in EtOH (150 mL) was heated under reflux for 72 h. The cooled mixture was filtered and the filtrate concentrated in vacuo. The residue was purified by HPLC to give Intermediate 18 7-isopropoxy-2-(tetrahydro-2H-pyran-4-yl)imidazo[1,2-a]pyridin-6-amine (1.44 g, 28.86% yield). LCMS (ESI) m/z 276.2 (M+H)⁺ Example 232: 4-(difluoromethyl)-N-(7-isopropoxy-2-(tetrahydro-2H-pyran-4-yl)imidazo[1,2-a]pyridin-6-yl)thiophene-2-carboxamide was prepared in a similar manner to that described for Step b: of Example 1, to a mixture of Intermediate 18 (30 mg, 108.95 μmol) and 4-(difluoromethyl)thiophene-2-carboxylic acid (29.12 mg, 163.43 μmol) in pyridine (544.75 μL) was added T3P® (346.66 mg, 544.75 μmol, 50% purity) and the reaction stirred at rt for 2 h. The mixture was partitioned between EtOAc and water, the layers separated, the aqueous extracted with EtOAc, washed with brine, dried over MgSO₄, filtered and evaporated under reduced pressure. The crude product was purified by TFA-modified mass-directed HPLC to give 4-(difluoromethyl)-N-(7-isopropoxy-2-(tetrahydro-2H-pyran-4-yl)imidazo[1,2-a]pyridin-6-yl)thiophene-2-carboxamide 2,2,2-trifluoroacetate (25.30 mg, 53.32% yield). LCMS (ESI) m/z 337.1 (M+H)⁺; ¹H NMR (500 MHz, DMSO-d₆) δ ppm 1.39 (br d, J=5.49 Hz, 12H), 1.64-1.77 (m, 4H), 1.95 (br d, J=11.60 Hz, 4H), 3.97 (br d, J=10.38 Hz, 14H), 4.95-5.02 (m, 4H), 7.25 (s, 2H), 7.97 (s, 1H), 8.20 (s, 1H), 8.30 (s, 1H), 9.14 (s, 1H), 10.07 (s, 2H)

Example 233: N-(7-isopropoxy-2-(tetrahydro-2H-pyran-4-yl)imidazo[1,2-a]pyridin-6-yl)-2-(2-methylpyridin-4-yl)oxazole-4-carboxamide; was prepared in a similar manner to that described for Example 232, to a mixture of Intermediate 18 (70 mg, 254.22 μmol) and 2-(2-methyl-4-pyridyl)oxazole-4-carboxylic acid (51.91 mg, 254.22 μmol) in pyridine (2.00 mL) was added T3P® (808.88 mg, 1.27 mmol, 50% purity) at rt and the reaction stirred overnight. The reaction was partitioned between with EtOAc (5 mL) and water (2 mL), the phases separated and the aqueous phase extracted with EtOAc (5 mL×2). The combined organic extracts were dried over anhydrous MgSO₄, filtered and the filtrate concentrated in vacuo. The crude was purified by TFA-modified mass-directed reverse phase HPLC (5-70% gradient of H₂O to MeCN) to give N-(7-isopropoxy-2-(tetrahydro-2H-pyran-4-yl)imidazo[1,2-a]pyridin-6-yl)-2-(2-methylpyridin-4-yl)oxazole-4-carboxamide 2,2,2-trifluoroacetate (39.30 mg, 26.86% yield) as a white solid. LCMS (ESI) m/z 462.3 (M+H)⁺; ¹H NMR (400 MHz, METHANOL-d₄) δ ppm 1.62 (d, J=6.02 Hz, 6H), 1.80-1.93 (m, 2H), 2.01-2.11 (m, 2H), 2.81-2.95 (m, 3H), 3.12-3.24 (m, 1H), 3.63 (td, J=11.73, 2.13 Hz, 2H), 4.09 (dt, J=9.66, 2.20 Hz, 2H), 5.09-5.11 (m, 1H), 7.36 (s, 1H), 7.79-7.91 (m, 1H), 8.35 (dd, J=6.02, 1.76 Hz, 1H), 8.38-8.47 (m, 1H), 8.85-8.94 (m, 1H), 8.94-9.02 (m, 1H), 9.62-9.77 (m, 1H).

Example 234: 6-(difluoromethyl)-N-(2-(3-methoxypropyl)imidazo[1,2-a]pyridin-6-yl)picolinamide; was prepared in a similar manner to that described for Intermediate 7 and Example 30, where 1-chloro-5-methoxypentan-2-one was used instead of 2-bromo-1-cyclopropyl-ethanone to yield 6-(difluoromethyl)-N-(2-(3-methoxypropyl)imidazo[1,2-a]pyridin-6-yl]picolinamide (30 mg, 12.17% yield) as a brown solid. LCMS (ESI) m/z 361.2 (M+H)⁺; ¹H NMR (500 MHz, CDCl₃) δ ppm 1.91-2.16 (m, 2H), 2.87 (t, J=7.6 Hz, 2H), 3.36 (s, 3H), 3.47 (t, J=6.4 Hz, 2H), 6.74 (t, J=5.2 Hz, 1H), 7.08 (d, J=9.6 Hz, 1H), 7.41 (s, 1H), 7.54 (d, J=9.6 Hz, 1H), 7.87 (d, J=7.7 Hz, 1H), 8.12 (t, J=7.7 Hz, 1H), 8.41 (d, J=7.7 Hz, 1H), 9.29 (s, 1H), 9.70 (s, 1H),

### Example 235: N-(2-(3-oxabicyclo[3.1.0]hexan-6-yl)-7-ethoxyimidazo[1,2-a]pyridin-6-yl)-1-(2,2,2-trifluoroethyl)-1H-pyrazole-3-carboxamide

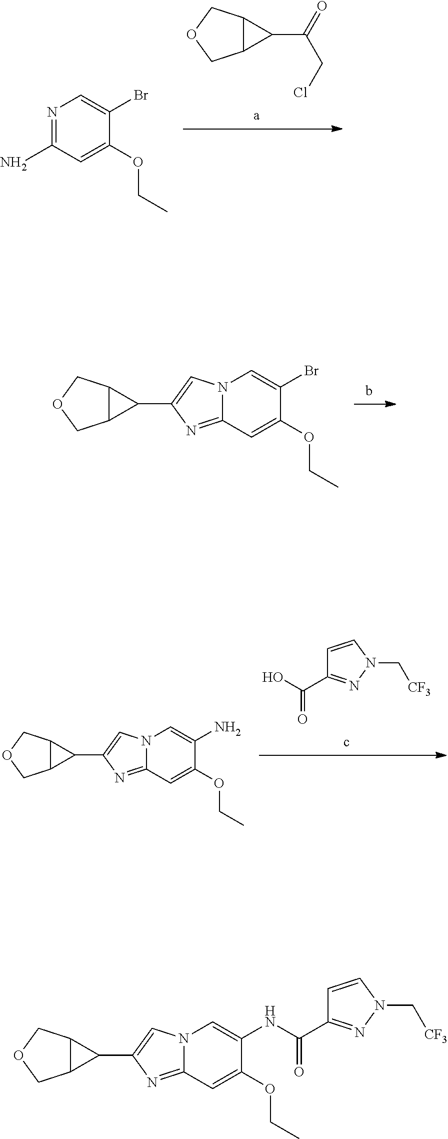 [SMILES: O=C(CCl)C1C2COCC21 ; CCOc1cc(N)ncc1Br ; CCOc1cc2nc(C3C4COCC43)cn2cc1Br ; O=C(O)c1ccn(CC(F)(F)F)n1 ; CCOc1cc2nc(C3C4COCC43)cn2cc1N ; CCOc1cc2nc(C3C4COCC43)cn2cc1NC(=O)c1ccn(CC(F)(F)F)n1]

Step a: A mixture of 5-bromo-4-ethoxypyridin-2-amine (1.69 g, 7.80 mmol), 1-(3-oxabicyclo[3.1.0]hexan-6-yl)-2-chloroethan-1-one (1.25 g, 7.80 mmol) and NaHCO₃ (1.97 g, 23.40 mmol) in EtOH (19.50 mL) was heated at 80° C. overnight. The mixture was cooled to rt, silica gel added and the mixture concentrated. The solid was purified via silica gel column chromatography to give 6-bromo-7-ethoxy-2-(3-oxabicyclo[3.1.0]hexan-6-yl)imidazo[1,2-a]pyridine (1.50 g, 59.50% yield). LCMS (ESI) m/z 324.9 (M+H)⁺;

Step b: A mixture of 6-bromo-7-ethoxy-2-(3-oxabicyclo[3.1.0]hexan-6-yl)imidazo[1,2-a]pyridine, NaOtBu (628.28 mg, 6.54 mmol), Pd₂(dba)₃ (128.29 mg, 140.10 μmol) and BINAP (232.62 mg, 373.60 μmol) in toluene (4.67 mL) was sealed and purged with N₂. Diphenylmethanimine (940.38 μL, 5.60 mmol) was added and the reaction heated at 100° C. overnight. The cooled reaction was concentrated onto silica gel and purified via column chromatography to give 2-(3-oxabicyclo[3.1.0]hexan-6-yl)-7-ethoxyimidazo[1,2-a]pyridin-6-amine (1.90 g, 96.07% yield).

Step c: DIPEA (74.76 mg, 578.49 μmol) was added to a mixture of 2-(3-oxabicyclo[3.1.0]hexan-6-yl)-7-ethoxyimidazo[1,2-a]pyridin-6-amine (50 mg, 192.83 μmol), HATU (73.51 mg, 192.83 μmol) and 1-(2,2,2-trifluoroethyl)pyrazole-3-carboxylic acid (37.43 mg, 192.83 μmol) in DMF (292.17 μL) at 0° C., and the reaction stirred overnight at rt. The reaction was quenched with brine, extracted with EtOAc (3×), the combined organic phases washed with brine, dried, filtered and concentrated in vacuo. The crude was purified by reverse phase HPLC to give N-(2-(3-oxabicyclo[3.1.0]hexan-6-yl)-7-ethoxyimidazo[1,2-a]pyridin-6-yl)-1-(2,2,2-trifluoroethyl)-1H-pyrazole-3-carboxamide 2,2,2-trifluoroacetate (3.30 mg, 3.12% yield) as a yellow solid. LCMS (ESI) m/z 436.0 (M+H)⁺; ¹H NMR (500 MHz, DMSO-d₆) δ ppm 1.48 (t, J=7.02 Hz, 3H), 1.95 (s, 1H), 2.18 (br d, J=3.05 Hz, 2H), 3.73 (s, 2H), 3.94 (d, J=8.55 Hz, 2H), 4.39 (d, J=6.71 Hz, 2H), 5.34 (br d, J=9.16 Hz, 2H), 6.91-7.07 (m, 1H), 7.26 (s, 1H), 7.97-8.18 (m, 2H), 9.44 (s, 1H).

Example 236: 6-cyclobutoxy-N-(7-ethoxy-2-(tetrahydro-2H-pyran-4-yl)imidazo[1,2-a]pyridin-6-yl)picolinamide was prepared in a similar manner to that described for Example 202, where Intermediate 24 was coupled through standard amide coupling to 6-(cyclobutoxy)pyridine-2-carboxylic acid to give 6-cyclobutoxy-N-(7-ethoxy-2-(tetrahydro-2H-pyran-4-yl)imidazo[1,2-a]pyridin-6-yl)picolinamide as a white solid (10 mg, 4.43% yield). LCMS (ESI) m/z 437.2 (M+H)⁺; ¹H NMR (400 MHz, CDCl₃) δ 1.60 (t, J=6.9 Hz, 3H), 1.63-2.07 (m, 6H), 2.12-2.29 (m, 2H), 2.51-2.63 (m, 2H), 2.90-2.99 (m, 1H), 3.55 (t, J=11.5 Hz, 2H), 4.06 (d, J=11.3 Hz, 2H), 4.14-4.29 (m, 2H), 5.29 (t, J=7.6 Hz, 1H), 6.87-6.94 (m, 2H), 7.19 (s, 1H), 7.64-7.87 (m, 2H), 9.51 (s, 1H), 10.17 (s, 1H).

Example 237: 6-(dimethylamino)-N-(7-ethoxy-2-(tetrahydro-2H-pyran-4-yl)imidazo[1,2-a]pyridin-6-yl)picolinamide was prepared in a similar manner to that described for Example 202, where Intermediate 24 was coupled through standard amide coupling to 6-bromopyridine-2-carboxylic acid to give 6-bromo-N-(7-ethoxy-2-tetrahydropyran-4-yl-imidazo[1,2-a]pyridin-6-yl)picolinamide (118 mg, 27.65% yield) as a white solid, which was used for the next step without further purification. LCMS (ESI) m/z 445.2 (M+H)⁺;

Step b: 6-Bromo-N-(7-ethoxy-2-tetrahydropyran-4-yl-imidazo[1,2-a]pyridin-6-yl)picolinamide (110 mg, 247.02 μmol) was dissolved in THF (10 mL) and excess aqueous dimethylamine, piperidine-2-carboxylic acid (1.60 mg, 12.35 μmol) and copper(I) iodide (2.35 mg, 12.35 μmol) were added. The mixture was heated in a steel autoclave at 80° C. overnight. The cooled mixture was concentrated in vacuo, the residue was dissolved in chloroform (10 mL), washed with water (5 mL), dried and evaporated. The crude was purified by prep HPLC (SunFire C18 100×19 mm, 5 um) eluting with water/MeOH 65-100% to give Example 237 6-(dimethylamino)-N-(7-ethoxy-2-(tetrahydro-2H-pyran-4-yl)imidazo[1,2-a]pyridin-6-yl)picolinamide (48 mg, 46% yield). LCMS (ESI) m/z 410.2 (M+H)⁺; ¹H NMR (400 MHz, DMSO-d₆) δ ppm: 1.44 (t, J=6.3 Hz, 3H), 1.59-1.74 (m, 2H), 1.89 (d, J=13.5 Hz, 2H), 2.80-2.93 (m, 1H), 3.14 (s, 6H), 3.45 (t, J=11.3 Hz, 2H), 3.91 (d, J=11.1 Hz, 2H), 4.20 (q, J=6.3 Hz, 2H), 6.95 (d, J=8.4 Hz, 1H), 7.02 (s, 1H), 7.36 (d, J=7.2 Hz, 1H), 7.63 (s, 1H), 7.74 (t, J=7.9 Hz, 1H), 9.46 (s, 1H), 10.48 (s, 1H).

Example 238: N-(2-cyclopropyl-7-ethoxyimidazo[1,2-a]pyridin-6-yl)-6-(tetrahydrofuran-3-yl)picolinamide was prepared in a similar manner to that described for Example 2, where Intermediate 5c was coupled through standard amide coupling to 6-(tetrahydrofuran-3-yl)picolinic acid to yield N-(2-cyclopropyl-7-ethoxyimidazo[1,2-a]pyridin-6-yl)-6-(tetrahydrofuran-3-yl)picolinamide (4.2 mg, 10.7 μmol, 3.3% yield). LCMS (ESI) m/z 393.0 (M+H)⁺; ¹H NMR (500 MHz, METHANOL-d₄) δ ppm 0.82 (m, 2H), 0.93 (m, 2H), 1.59 (t, J=7.0 Hz, 3H), 1.96 (m, 1H), 2.34 (m, 1H), 2.42 (m, 1H), 3.73 (m, 1H), 4.00 (m, 2H), 4.10 (m, 1H), 4.23 (m, 3H), 6.80 (s, 1H), 7.40 (s, 1H), 7.55 (d, J=7.0 Hz, 1H), 7.95 (t, J=7.0 Hz, 1H), 8.04 (d, J=7.0 Hz, 1H), 9.40 (s, 1H). 6-(tetrahydrofuran-3-yl)picolinic acid was prepared as follows:

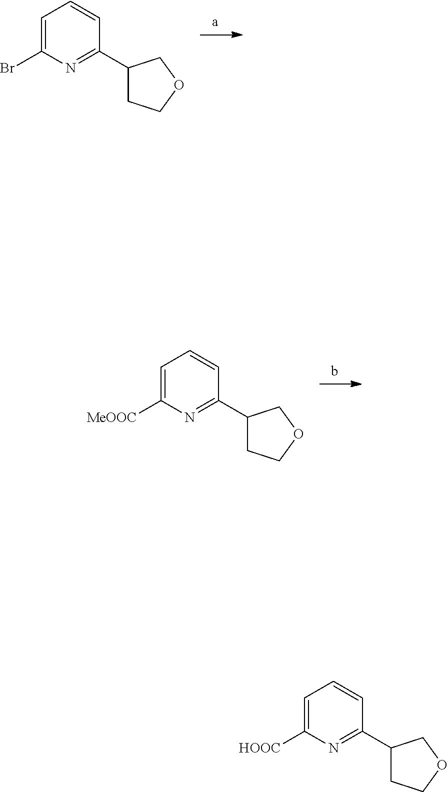 [SMILES: Brc1cccc(C2CCOC2)n1 ; COC(=O)c1cccc(C2CCOC2)n1 ; O=C(O)c1cccc(C2CCOC2)n1]

Step a: A mixture of 2-bromo-6-(tetrahydrofuran-3-yl)pyridine (1.24 g, 5.45 mmol), TEA (661.26 mg, 6.54 mmol) and Pd(dppf)Cl₂ (199.23 mg, 272.50 μmol) in MeOH (100 mL) was heated at 110° C. for 24 h under CO atmosphere (38000 Torr) in a sealed vessel. The reaction mixture was concentrated in vacuo, the residue suspended in MTBE and filtered through a silica gel pad. The filtrate was evaporated under reduced pressure to give methyl 6-(tetrahydrofuran-3-yl)picolinate (400 mg, 35.42% yield) as a reddish oil. ¹H NMR: (500 MHz, CDCl₃) δ ppm 2.18 (m, 1H), 2.45 (m, 1H), 3.75 (m, 1H), 3.93 (m, 2H), 4.00 (s, 3H), 4.11 (m, 1H), 4.18 (m, 1H), 7.45 (d, J=7.5 Hz, 1H), 7.79 (t, J=7.5 Hz, 1H), 7.98 (d, J=7.5 Hz, 1H).

Step b: A solution of LiOH.H₂O (242.98 mg, 5.79 mmol) in water (10 mL) was added in one portion to a solution of methyl 6-(tetrahydrofuran-3-yl)picolinate (400 mg, 1.93 mmol) in THF (60 mL) and the reaction stirred overnight at rt. The reaction was concentrated in vacuo and the residue was dissolved in water (30 mL). This solution was washed with DCM (2×20 mL) and acidified by the addition of sodium hydrogen sulfate (1.85 g, 15.44 mmol) in water (10 mL). The mixture was extracted with DCM (2×30 mL), the organic phase was dried over Na₂SO₄ and evaporated under reduced pressure to give 6-(tetrahydrofuran-3-yl)picolinic acid (40 mg, 10.73% yield) as brown oil. ¹H NMR: (500 MHz, CDCl₃) δ ppm 2.20 (m, 1H), 2.43 (m, 1H), 3.69 (m, 1H), 3.95 (m, 1H), 4.11 (m, 2H), 4.20 (m, 1H), 7.51 (d, J=7.5 Hz, 1H), 7.90 (t, J=7.5 Hz, 1H), 8.08 (d, J=7.5 Hz, 1H)

Example 239: 6-(1,2-difluoroethyl)-N-(7-ethoxy-2-(tetrahydro-2H-pyran-4-yl)imidazo[1,2-a]pyridin-6-yl)picolinamide was prepared in a similar manner to that described for Example 2, where Intermediate 24 was coupled through standard amide coupling to methyl 6-(1,2-Difluoroethyl)picolinic acid to yield (6.9 mg, 6.9%) of Example 239. LCMS (ESI) m/z 432.4 (M+H)⁺; ¹H NMR (400 MHz, METHANOL-d₄) δ ppm: 1.59 (t, J=6.9 Hz, 3H), 1.74-1.86 (m, 2H), 1.93-2.02 (m, 2H), 2.87-2.98 (m, 1H), 3.52-3.69 (m, 2H), 4.03 (d, J=11.5 Hz, 2H), 4.24 (q, J=6.9 Hz, 2H), 4.88-5.10 (m, 2H), 5.74-6.05 (m, 1H), 6.86 (s, 1H), 7.47 (s, 1H), 7.80 (d, J=7.7 Hz, 1H), 8.12 (t, J=7.8 Hz, 1H), 8.20 (d, J=7.8 Hz, 1H), 9.42 (s, 1H). 6-(1,2-Difluoroethyl)picolinic acid was prepared as follows: To a mixture of methyl 6-bromopicolinate (5.00 g, 23.1 mmol) and tributyl(vinyl)stannane (8.76 g, 27.6 mmol) in dioxane (150 mL) were added bis(triphenylphosphine)palladium(II) dichloride (1.62 g, 2.31 mmol) and the mixture degassed with N₂, then stirred at 100° C. for 16 h. The cooled reaction was quenched with KF aq. (200 mL) and extracted with EtOAc (200 mL×3). The combined organic extracts were washed with brine (300 mL), dried over Na₂SO₄ and filtered. The filtrate was concentrated in vacuo and the residue was purified by column chromatography on silica gel using Combiflash® (PE/EtOAc=10/1 to 3/1) to give methyl 6-vinylpicolinate (3.50 g, 93% yield) as yellow oil. ¹H NMR (500 MHz, CDCl₃) δ ppm: 4.02 (s, 3H), 5.60 (d, J=11.0 Hz, 1H), 6.23 (d, J=18.0 Hz, 1H), 7.00-6.80 (m, 1H), 7.62 (d, J=7.5 Hz, 1H), 7.81 (t, J=8.0 Hz, 1H), 8.01 (d, J=7.5 Hz, 1H).

Step b: To a solution of methyl 6-vinylpicolinate (500 mg, 3.06 mmol) in water (2 mL) and tBuOH (2 mL) was added N-methylmorpholine-N-oxide (430 mg, 3.67 mmol) and K₂OsO₄ (56 mg, 0.153 mmol) and the reaction stirred at 10-15° C. for 1 h. The reaction was quenched with saturated Na₂SO₃ aq. (50 mL) and extracted with DCM/MeOH (10/1, 50 mL×10). The combined organic extracts were dried over Na₂SO4, filtered and the filtrate concentrated in vacuo to give methyl 6-(1,2-dihydroxyethyl)picolinate (400 mg, 66% yield) as a yellow oil. LCMS (ESI) m/z 197.8 (M+H)⁺;

Step c: To a solution of methyl 6-(1,2-dihydroxyethyl)picolinate (400 mg, 2.03 mmol) in DCM (20 mL) was added DAST (1.35 g, 6.09 mmol) at 0° C. and the reaction stirred at 10-15° C. for 16 h. The reaction was quenched with saturated NaHCO₃ aq. (30 mL) and extracted with DCM (30 mL×3). The combined organic extracts were washed with brine (50 mL), dried over Na₂SO₄, filtered and concentrated in vacuo. The residue was purified by silica gel column chromatography using Combiflash® (PE/EtOAc=10/1 to 3/1) to give methyl 6-(1,2-difluoroethyl)picolinate (300 mg, 73% yield) as an off-white solid. ¹H NMR (500 MHz, CHLOROFORM-d) δ ppm: 4.02 (m, 3H), 4.70-5.10 (m, 2H), 5.80-6.00 (m, 1H), 7.76 (d, J=8.0 Hz, 1H), 7.95 (t, J=8.0 Hz, 1H), 8.12 (d, J=8.0 Hz, 1H)

Step d: To a solution of methyl 6-(1,2-difluoroethyl)picolinate (300 mg, 1.49 mmol) in MeOH (5 mL) and water (1 mL) was added LiOH (107 mg, 4.47 mmol) and the reaction stirred at 10-15° C. for 12 h. The mixture was acidified to pH with 1M HCl and then concentrated in vacuo. The remaining aqueous layer was extracted with EtOAc (30 mL×3), the combined organic extracts washed with brine (50 mL), dried over Na₂SO₄ and filtered. The filtrate was evaporated under reduced pressure to give 6-(1,2-difluoroethyl)picolinic acid (250 mg, 90% yield) as yellow oil. ¹H NMR: (500 MHz, DMSO-d₆) δ ppm: 4.70-5.10 (m, 2H), 5.90-6.10 (m, 2H), 7.70-7.80 (m, 1H), 8.00-8.15 (m, 2H).

Example 240: N-(7-ethoxy-2-(tetrahydro-2H-pyran-4-yl)imidazo[1,2-a]pyridin-6-yl)-2,3-dihydrofuro[2,3-b]pyridine-5-carboxamide was prepared in a similar manner to that described for Example 202, where Intermediate 24 was coupled through standard amide coupling to 2,3-dihydrofuro[2,3-b]pyridine-6-carboxylic acid to give N-(7-ethoxy-2-(tetrahydro-2H-pyran-4-yl)imidazo[1,2-a]pyridin-6-yl)-2,3-dihydrofuro[2,3-b]pyridine-5-carboxamide, 24.5 mg, 22.1% yield. LCMS (ESI) m/z 409.2 (M+H)⁺; ¹H NMR (400 MHz, CHLOROFORM-d) δ ppm: 1.56 (t, J=7.0 Hz, 3H), 1.76-1.89 (m, 2H), 1.95-2.05 (m, 2H), 2.89-3.00 (m, 1H), 3.27-3.37 (m, 2H), 3.49-3.60 (m, 2H), 3.94-4.06 (m, 2H), 4.18 (q, J=7.0 Hz, 2H), 4.66-4.75 (m, 2H), 6.84 (s, 1H), 7.16 (s, 1H), 7.63 (d, J=7.4 Hz, 1H), 7.78 (d, J=7.4 Hz, 1H), 9.40 (s, 1H), 10.25 (s, 1H). 3-dihydrofuro[2,3-b]pyridine-6-carboxylic acid was prepared by a mixture of methyl 2,3-dihydrofuro[2,3-b]pyridine-6-carboxylate (1.3 g, 7.26 mmol), K₂CO₃ (2.51 g, 18.1 mmol) in H₂O (15 mL) and EtOH (10 mL) was stirred at rt for 48 h. The mixture was evaporated in vacuo, H₂O and activated carbon added and the mixture filtered. The filtrate was acidified using conc. HCl to pH ˜4-5 and the precipitate was filtered off, washed with water and air-dried. The solid was recrystallized from 2-propanol, to give 2,3-dihydrofuro[2,3-b]pyridine-6-carboxylic acid 280 mg, as a pale-yellow solid. LCMS (ESI) m/z 166 (M+H)⁺; ¹H NMR (500 MHz, DMSO-d₆) δ ppm: 3.21-3.29 (m, 2H), 4.54-4.61 (m, 2H), 7.50 (d, J=7.3 Hz, 1H), 7.67 (d, J=7.3 Hz, 1H). water/MeOH 66%

### Example 241: N-(2-(2-cyanopropan-2-yl)-7-ethoxyimidazo[1,2-a]pyridin-6-yl)-2-methylthiazole-4-carboxamide

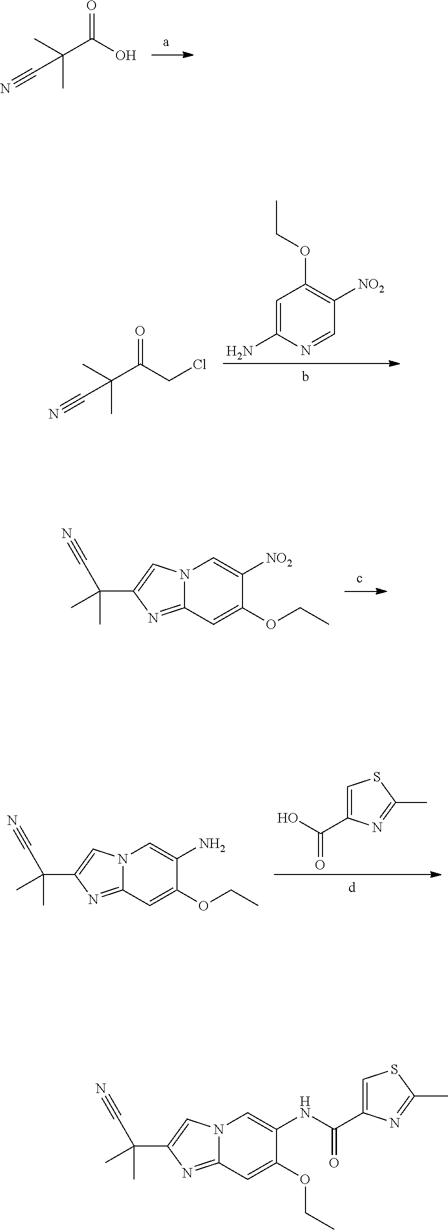

Step a: Oxalyl chloride (1.23 g, 9.72 mmol) was added to a solution of 2-cyano-2-methyl-propanoic acid 1 (1.00 g, 8.84 mmol) in benzene (20 mL) and the reaction stirred at rt for 20 h. The reaction was evaporated under reduced pressure and the crude 2-cyano-2-methyl-propanoyl chloride dissolved in benzene (30 mL). A solution of diazomethane (958.51 mg, 22.80 mmol) in MTBE (30 mL) was added at 0° C. and the reaction stirred at 15° C. for 4 h. Aq. HCl (15 mL, 15%) was added dropwise, maintaining the temperature below 15° C. and the layers separated. The organic phase was evaporated under reduced pressure and the product distilled off to give 4-chloro-2,2-dimethyl-3-oxo-butanenitrile (1.00 g, 75.31% yield). ¹H NMR (500 MHz, CHLOROFORM-d) δ ppm: 1.60 (s, 6H), 4.61 (s, 2H).

Step b: 4-Chloro-2,2-dimethyl-3-oxo-butanenitrile (1.00 g, 6.87 mmol) and 4-ethoxy-5-nitropyridin-2-amine (1.26 g, 6.87 mmol) were heated at 105° C. in propionitrile (25 mL) for 48 h. The solvent was evaporated under reduced pressure and the residue partitioned between EtOAc (50 mL) and 5% aq. Na₂CO₃ (20 mL) and the layers separated. The organic solution was concentrated under reduced pressure, and the residue purified by column chromatography on silica gel eluting with DCM to give 2-(7-ethoxy-6-nitro-imidazo[1,2-a]pyridin-2-yl)-2-methylpropanenitrile (201.45 mg, 10.69% yield). ¹H NMR (400 MHz, CHLOROFORM-d) 6 ppm: 1.51 (t, J=6.9 Hz, 3H), 1.78 (s, 6H), 4.17 (q, J=6.9 Hz, 2H), 6.96 (s, 1H), 7.55 (s, 1H), 8.84 (s, 1H).

Step c: A mixture of 2-(7-ethoxy-6-nitro-imidazo[1,2-a]pyridin-2-yl)-2-methylpropanenitrile (200 mg, 0.510 mmol), iron powder (570.17 mg, 10.21 mmol), ammonium chloride (54.61 mg, 1.02 mmol) and conc. HCl (catalytic) in EtOH (20 mL) was heated at 50° C. for 20 h. The reaction mixture was filtered, and the solvent evaporated under reduced pressure. The residue was suspended in EtOAc (20 mL), washed with water (10 mL) and the solvent evaporated under reduced pressure to give 2-(6-amino-7-ethoxyimidazo[1,2-a]pyridin-2-yl)-2-methylpropanenitrile (100 mg, 56.14% yield). LCMS (ESI) m/z 245.2 (M+H)⁺;

Step d: 2-Methylthiazole-4-carboxylic acid (38.68 mg, 0.27 mmol), TEA (27.34 mg, 0.27 mmol) and HATU (103 mg, 0.27 mmol) were dissolved in MeCN (5 mL) and the solution stirred at rt for 1 h. 2-(6-Amino-7-ethoxyimidazo[1,2-a]pyridin-2-yl)-2-methylpropanenitrile (33 mg, 0.135 mmol) was added and the reaction heated at 60° C. for 48 h. The solvent was evaporated under reduced pressure, the residue diluted with EtOAc (20 mL) and washed with water (10 mL). The solvent was evaporated under reduced pressure, and the residue purified by prep HPLC to give N-(2-(2-cyanopropan-2-yl)-7-ethoxyimidazo[1,2-a]pyridin-6-yl)-2-methylthiazole-4-carboxamide (12.20 mg, 24.45% yield). LCMS (ESI) m/z 370.2 (M+H)⁺; ¹H NMR (400 MHz, CHLOROFORM-d) δ ppm 1.57 (t, J=7.0 Hz, 3H), 1.78 (s, 6H), 2.75 (s, 3H), 4.19 (q, J=7.0 Hz, 2H), 6.90 (s, 1H), 7.43 (s, 1H), 8.03 (s, 1H), 9.39 (s, 1H), 9.77 (s, 1H).

Example 242: 6-(difluoromethyl)-N-(7-ethoxy-2-(3-methoxypropyl)imidazo[1,2-a]pyrimidin-6-yl)picolinamide was prepared in a manner similar to Example 2 where 1-chloro-5-methoxypentan-2-one (1.13 g, 7.50 mmol) and 5-bromo-4-ethoxypyrimidin-2-amine (818.05 mg, 3.75 mmol) are used to give 7-ethoxy-2-(3-methoxypropyl)imidazo[1,2-a]pyrimidin-6-amine hydrochloride (50 mg, 41% yield) as a yellow solid. LCMS (ESI) m/z 264 (M+H)⁺; followed by standard peptide coupling conditions with 6-(difluoromethyl)picolinic acid to yield 6-(difluoromethyl)-N-[7-ethoxy-2-(3-methoxypropyl)imidazo[1,2-a]pyrimidin-6-yl]pyridine-2-carboxamide (1.70 mg, 4.19 μmol, 2.74% yield) as a yellow solid. LCMS (ESI) m/z 406.0 (M+H)⁺; ¹H NMR (400 MHz, METHANOL-d₄) δ ppm 1.55 (t, J=7.1 Hz, 3H), 1.94-2.02 (m, 2H), 2.72-2.78 (m, 2H), 3.33 (s, 1H), 3.41-3.49 (m, 2H), 4.63 (q, J=7.1 Hz, 2H), 6.85 (t, J=54.0 Hz, 1H), 7.40 (s, 1H), 7.94 (d, J=7.8 Hz 1H), 8.24 (t, J=7.8 Hz, 1H), 8.36 (d, J=7.8 Hz, 1H), 9.59 (s, 1H).

### Example 243: N-(2-cyclopropyl-7-ethoxyimidazo[1,2-a]pyrimidin-6-yl)-6-(difluoromethyl)picolinamide

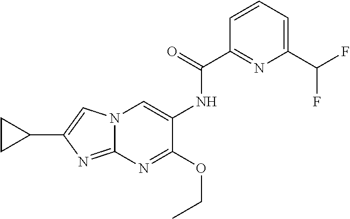 [SMILES: CCOc1nc2nc(C3CC3)cn2cc1NC(=O)c1cccc(C(F)F)n1]

N-(2-cyclopropyl-7-ethoxyimidazo[1,2-a]pyrimidin-6-yl)-6-(difluoromethyl)picolinamide was prepared from 4-ethoxypyrimidin-2-amine in a similar fashion to Example 242 6-(difluoromethyl)-N-(7-ethoxy-2-(3-methoxypropyl)imidazo[1,2-a]pyrimidin-6-yl)picolinamide (above), except 2-chloro-1-cyclopropyl-ethanone was used instead of 1-chloro-5-methoxypentan-2-one, (5.90 mg, 4.06% yield). LCMS (ESI) m/z 374.2 (M+H)⁺; ¹H NMR (400 MHz, METHANOL-d₄) δ ppm 0.80-0.99 (m, 4H), 1.54 (t, J=7.1 Hz, 3H), 1.90-2.01 (m, 1H), 4.63 (q, J=7.1 Hz, 2H), 6.85 (t, J=54 Hz, 1H), 7.41 (s, 1H), 7.94 (d, J=7.9 Hz, 1H), 8.24 (t, J=7.9 Hz, 1H), 8.35 (d, J=7.9 Hz, 1H), 9.57 (s, 1H).

Example 244: 2-cyclopropyl-N-(8-methoxy-2-(tetrahydro-2H-pyran-4-yl)imidazo[1,2-a]pyridin-6-yl)thiazole-4-carboxamide was prepared in a manner similar to scheme V Example 202 where 3-methoxy-5-nitropyridin-2-amine was used instead of 4-ethoxy-5-nitropyridin-2-amine to give 8-methoxy-2-(tetrahydro-2H-pyran-4-yl)imidazo[1,2-a]pyridin-6-amine (3.50 g, 44.6% yield, 86.3%) as a brown solid and was used without further purification. LCMS (ESI) m/z 248.2 (M+H); ¹H NMR (500 MHz, CDCl₃): δ 1.73-1.87 (m, 2H), 1.96-2.07 (m, 2H), 2.95-3.01 (m, 1H), 3.50-3.55 (m, 2H), 3.93 (s, 3H), 3.97-4.08 (m, 2H), 5.53 (br s, 2H), 6.05 (s, 1H), 7.12 (s, 1H), 7.27 (s, 1H). Followed by standard peptide coupling conditions with 2-cyclopropylthiazole-4-carboxylic acid to yield 2-cyclopropyl-N-(8-methoxy-2-(tetrahydro-2H-pyran-4-yl)imidazo[1,2-a]pyridin-6-yl)thiazole-4-carboxamide as brown solid, (80.6 mg, 20.0% yield). LCMS (ESI) m/z 399.2 (M+H)⁺; ¹H NMR (400 MHz, DMSO-d₆): δ ppm: 1.09-1.13 (m, 2H), 1.17-1.22 (m, 2H), 1.62-1.72 (m, 2H), 1.87-1.91 (m, 2H), 2.78-2.98 (m, 1H), 3.40-3.49 (m, 2H), 3.91 (s, 3H), 3.86-3.95 (m, 2H), 7.03 (s, 1H), 7.75 (s, 1H), 8.19 (s, 1H), 8.88 (s, 1H), 10.00 (br s, 1H).

### Example 245: N-(2-(2-cyanopropan-2-yl)-7-ethoxyimidazo[1,2-a]pyridin-6-yl)-6-(1,2-difluoroethyl)picolinamide

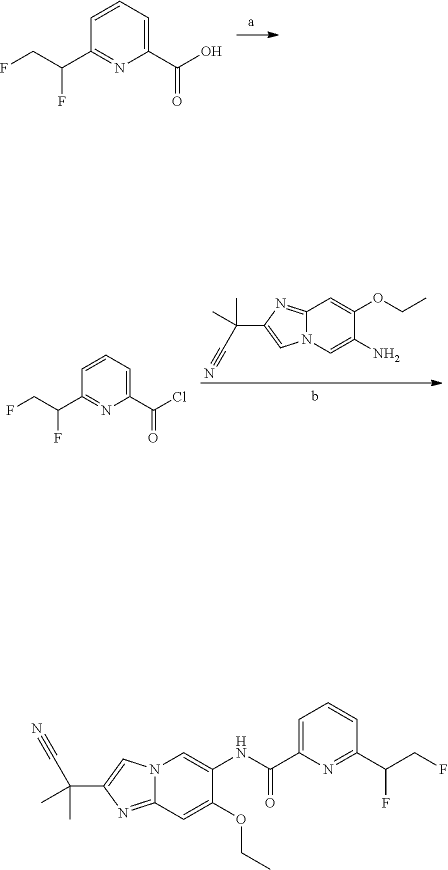

Step a: 6-(1,2-Difluoroethyl)pyridine-2-carboxylic acid (150 mg, 0.801 mmol) and oxalyl chloride (111.91 mg, 0.881 mmol) were stirred in benzene (5 mL) at rt overnight. The solvent was removed under reduced pressure to give 6-(1,2-difluoroethyl)picolinoyl chloride (175 mg), which was used for the next step without further purification.

Step b: A solution of Example 241, step c:2-(6-amino-7-ethoxyimidazo[1,2-a]pyridin-2-yl)-2-methylpropanenitrile (50 mg, 0.204 mmol) and 6-(1,2-difluoroethyl)picolinoyl chloride (63.12 mg, 0.307 mmol) in DCM (20 mL) was cooled to −40° C. TEA (42 mg, 0.409 mmol) was added, and the reaction stirred at rt for 3 h. The mixture was washed with water (20 mL) and evaporated under reduced pressure. The crude was purified by HPLC to give N-(2-(2-cyanopropan-2-yl)-7-ethoxyimidazo[1,2-a]pyridin-6-yl)-6-(1,2-difluoroethyl)picolinamide (21.20 mg, 25.05% yield). LCMS (ESI) m/z 414.2 (M+H); ¹H NMR (400 MHz, DMSO-d₆) δ ppm 1.51 (t, J=6.9 Hz, 3H), 1.71 (s, 6H), 4.24 (q, J=6.9 Hz, 2H), 4.89-5.25 (m, 2H), 6.08 (m, 1H), 7.16 (s, 1H), 7.87 (d, J=7.3 Hz, 1H), 7.94 (s, 1H), 8.15-8.29 (m, 2H), 9.46 (s, 1H), 10.45 (s, 1H).

### Example 246: 6-(difluoromethyl)-N-(7-ethoxy-2-(1-methyl-6-oxopiperidin-2-yl)imidazo[1,2-a]pyridin-6-yl)picolinamide

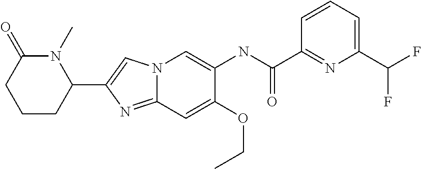

6-(Difluoromethyl)-N-(7-ethoxy-2-(1-methyl-6-oxopiperidin-2-yl)imidazo[1,2-a]pyridin-6-yl)picolinamide was prepared from 1-methyl-6-oxopiperidine-2-carboxylic acid and Intermediate 3c: N-(6-amino-4-ethoxypyridin-3-yl)-6-(difluoromethyl)picolinamide, following the procedure described in Example 75, 18.6 mg, 2.64% yield. LCMS (ESI) m/z 444.2 (M+H)⁺; ¹H NMR 400 MHz, METHANOL-d₄): δ ppm 1.60 (t, J=7.0 Hz, 3H), 1.48-1.88 (m, 2H), 2.04-2.25 (m, 2H), 2.38-2.49 (m, 2H), 2.91 (s, 3H), 4.29 (q, J=7.0 Hz, 2H), 4.70-4.78 (m, 1H), 6.94 (t, J=55.4 Hz, 1H), 6.97 (s, 1H), 7.63 (s, 1H), 7.92 (d, J=7.8 Hz, 1H), 8.23 (t, J=7.8 Hz, 2H), 8.36 (d, J=7.8 Hz, 1H), 9.49 (s, 1H).

### Example 247: 6-(difluoromethyl)-N-(7-ethoxy-2-(1-methyl-2-oxabicyclo[2.1.1]hexan-4-yl)imidazo[1,2-a]pyridin-6-yl)picolinamide

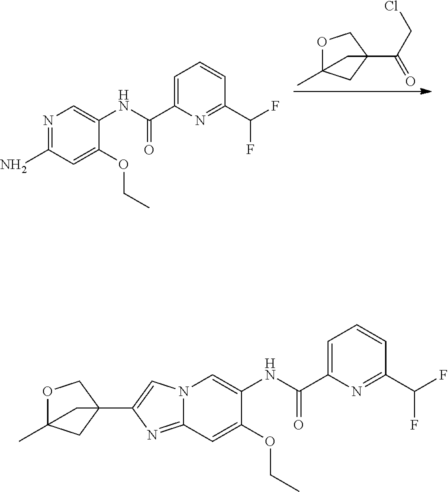

A mixture of 2-chloro-1-(1-methyl-2-oxabicyclo[2.1.1]hexan-4-yl)ethan-1-one (200 mg, 1.15 mmol), Intermediate 3c: N-(6-amino-4-ethoxypyridin-3-yl)-6-(difluoromethyl)picolinamide (354.52 mg, 1.15 mmol) and NaHCO₃ (96.61 mg, 1.15 mmol) in EtOH (15 mL) was heated under reflux for 12 h. The mixture was cooled to rt and concentrated under reduced pressure. The residue was dissolved in EtOAc (10 mL), washed with water (10 mL), dried and evaporated under reduced pressure. The crude product was purified by prep HPLC (SunFire C18 150×19 Sum) eluting with (water/MeOH from 65%-70%) to give 6-(difluoromethyl)-N-(7-ethoxy-2-(1-methyl-2-oxabicyclo[2.1.1]hexan-4-yl)imidazo[1,2-a]pyridin-6-yl)picolinamide (106 mg, 20.87% yield). LCMS (ESI) m/z 429.2 (M+H)⁺; ¹H NMR (400 MHz, DMSO-d₆) δ ppm: 1.42 (s, 3H), 1.49 (t, J=6.9 Hz, 3H), 1.69-1.77 (m, 2H), 1.91-1.99 (m, 2H), 3.86 (s, 2H), 4.22 (q, J=6.9 Hz, 2H), 7.07 (s, 1H), 7.11 (t, J=54.0 Hz, 1H), 7.76 (s, 1H), 8.00 (dd, J=5.7, 3.3 Hz, 1H), 8.16-8.45 (m, 2H), 9.41 (s, 1H), 10.48 (s, 1H)

### Example 248: 6-(difluoromethyl)-N-(7-ethoxy-2-(1-methyl-2-oxabicyclo[2.2.1]heptan-4-yl)imidazo[1,2-a]pyridin-6-yl)picolinamide

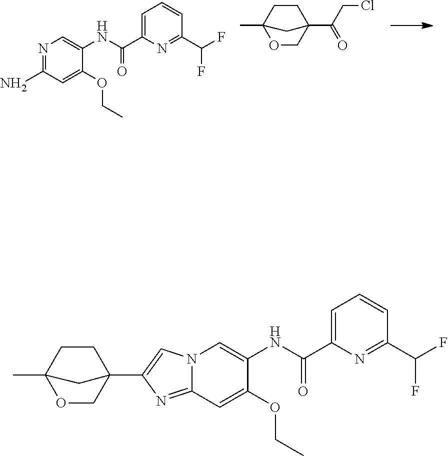

A mixture of Intermediate 3c: N-(6-amino-4-ethoxypyridin-3-yl)-6-(difluoromethyl)picolinamide (81.71 mg, 0.265 mmol), 2-chloro-1-(1-methyl-2-oxabicyclo[2.2.1]heptan-4-yl)ethan-1-one (50 mg, 0.265 mmol) and NaHCO₃ (66.80 mg, 0.795 mmol) in EtOH (662.60 μL) was heated at 80° C. overnight. The cooled reaction was filtered, concentrated in vacuo and the residue purified by TFA modified mass-directed HPLC, to provide 6-(difluoromethyl)-N-(7-ethoxy-2-(1-methyl-2-oxabicyclo[2.2.1]heptan-4-yl)imidazo[1,2-a]pyridin-6-yl)picolinamide 2,2,2-trifluoroacetate (9.10 mg, 6.18% yield). LCMS (ESI) m/z 442.9 (M+H); ¹H NMR (500 MHz, DMSO-d₆): δ ppm 1.41 (s, 3H), 1.56 (t, J=7.02 Hz, 3H), 1.70-1.78 (m, 1H), 1.84 (td, J=12.06, 4.58 Hz, 1H), 1.94 (s, 2H), 1.98-2.11 (m, 2H), 3.84 (d, J=6.71 Hz, 1H), 3.92 (dd, J=6.41, 3.36 Hz, 1H), 4.33-4.50 (m, 2H), 6.89-7.35 (m, 2H), 8.06 (dd, J=7.32, 1.83 Hz, 1H), 8.27-8.43 (m, 2H), 9.72 (br s, 1H), 10.63 (s, 1H),

### Example 249: N-(2-((1S*,6R*)-3-oxabicyclo[4.1.0]heptan-6-yl)-7-ethoxyimidazo[1,2-a]pyridin-6-yl)-6-(difluoromethyl)picolinamide

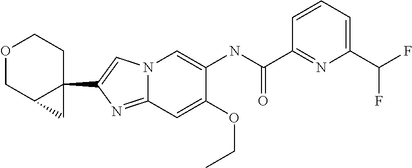

N-(2-((1S*,6R*)-3-oxabicyclo[4.1.0]heptan-6-yl)-7-ethoxyimidazo[1,2-a]pyridin-6-yl)-6-(difluoromethyl)picolinamide was prepared from cis 3-oxabicyclo[4.1.0]heptane-6-carboxylic acid and Intermediate 3c: N-(6-amino-4-ethoxypyridin-3-yl)-6-(difluoromethyl)picolinamide following the procedure described in Example 82. ¹H NMR (400 MHz, DMSO-d₆): δ ppm 0.89-0.91 (m, 1H), 1.24-1.28 (m, 1H), 1.42-1.51 (m, 4H), 1.95-2.02 (m, 1H), 2.27-2.33 (m, 1H), 3.35-3.37 (m, 1H), 3.49-3.55 (m, 1H), 3.72-3.80 (m, 1H), 3.82-3.96 (m, 1H), 4.17-4.22 (m, 2H), 6.89-7.18 (m, 2H), 7.72 (s, 1H), 7.99 (t, J=4.4 Hz, 1H), 8.31 (d, J=4.7 Hz, 2H), 9.36 (s, 1H), 10.46 (s, 1H).

### Example 250: N-(2-((2R*,6S*)-2,6-dimethyltetrahydro-2H-pyran-4-yl)-7-ethoxyimidazo[1,2-a]pyridin-6-yl)-6-(trifluoromethyl)picolinamide

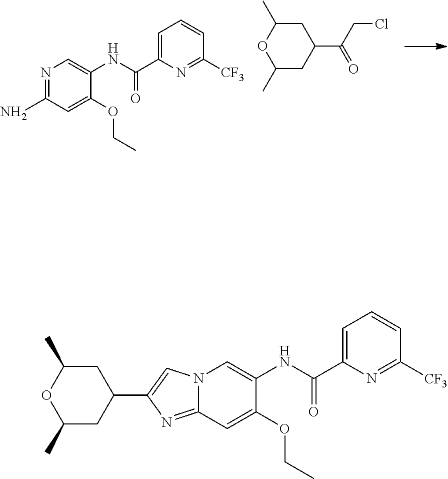

A mixture of Intermediate 4:N-(6-amino-4-ethoxypyridin-3-yl)-6-(trifluoromethyl)picolinamide (171.12 mg, 524.47 μmol), 2-chloro-1-(2,6-dimethyltetrahydropyran-4-yl)ethan-1-one (100 mg, 524.47 μmol) and NaHCO₃ (132.18 mg, 1.57 mmol) in EtOH (1.31 mL) was sealed and the reaction heated at 85° C. for 18 h. The cooled reaction was filtered, and the filtrate concentrated in vacuo. The residue was purified by prep HPLC to give N-[2-(2,6-dimethyltetrahydropyran-4-yl)-7-ethoxyimidazo[1,2-a]pyridin-6-yl]-6-(trifluoromethyl)picolinamide (46 mg, 18.97% yield). This compound was further purified by SFC using a CHIRALPAK-OX—H 30×250 mm, 5 μm column, eluting with 30% EtOH w/0.1% DEA in CO₂ at 100 mL/min, ABPR 120 bar, column temp 40 C) to obtain diastereoisomer 1, (cis stereochem) Rf=1.86 min. ¹H NMR (400 MHz, METHANOL-d₄) δ ppm 1.26 (d, J=6.27 Hz, 6H) 1.38 (d, J=11.29 Hz, 3H), 1.61 (t, J=6.90 Hz, 4H), 2.01-2.08 (m, 2H), 2.95-3.07 (m, 1H), 3.61-3.81 (m, 3H), 4.29 (d, J=6.78 Hz, 3H), 6.92 (s, 1H), 7.49 (s, 1H), 8.09 (d, J=7.78 Hz, 1H), 8.34 (s, 1H), 8.44-8.57 (m, 1H), 9.48 (s, 1H). Further elution provided the second diastereoisomer, (trans stereochem) at 2.37 min.

### Example 251 and 252: N-(7-ethoxy-2-((tetrahydrofuran-3-yl)methyl)imidazo[1,2-a]pyridin-6-yl)-2-(1-methyl-1H-pyrazol-4-yl)oxazole-4-carboxamide, Enantiomer 1 and 2

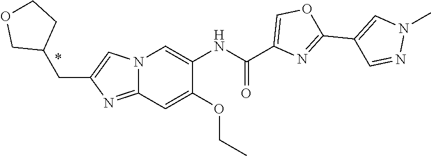 [SMILES: CCOc1cc2nc(CC3CCOC3)cn2cc1NC(=O)c1coc(-c2cnn(C)c2)n1]

Example 22, N-(7-ethoxy-2-((tetrahydrofuran-3-yl)methyl)imidazo[1,2-a]pyridin-6-yl)-2-(1-methyl-1H-pyrazol-4-yl)oxazole-4-carboxamide was further purified by SFC using a CHIRALPAK AD-H 30×250 mm, 5 μm column, eluting with 40% EtOH w/0.1% DEA in CO₂, flow rate 100 mL/min, ABPR 120 bar, column temp 40 C) to give enantiomer 1, Rf=3.63 min. Further elution provided enantiomer 2, Rf=4.01 min.

### Example 253 and 254: 6-(difluoromethyl)-N-(7-ethoxy-2-((1R*,2S*)-2-fluorocyclopropyl)imidazo[1,2-a]pyridin-6-yl)picolinamide and 6-(difluoromethyl)-N-(7-ethoxy-2-((1S*, 2R*)-2-fluorocyclopropyl)imidazo[1,2-a]pyridin-6-yl)picolinamide

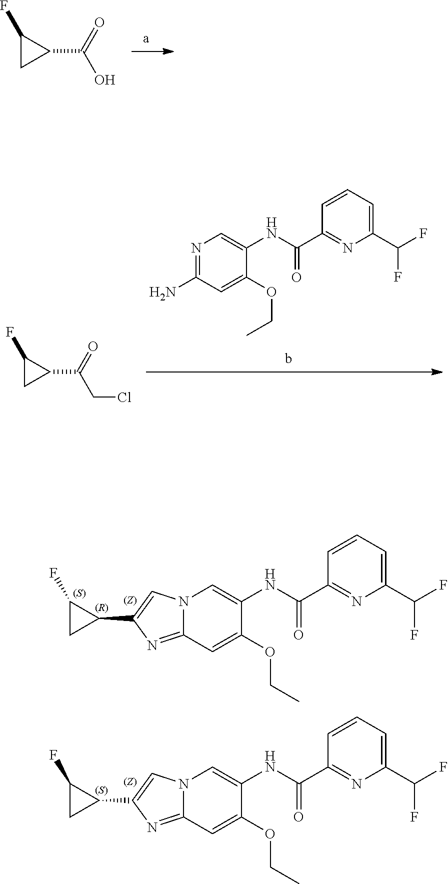

Step a: To a solution of trans 2-fluorocyclopropane-1-carboxylic acid (500 mg, 4.80 mmol) in DCM (5 mL) was added SOCl₂ (571.53 mg, 4.80 mmol) and DMF (2 drops) and the reaction stirred at 0° C. for 1 h. The mixture was concentrated in vacuo, the residue diluted with THF (5 mL) and MeCN (3 mL) and the solution cooled to 0° C. TMSCHN₂ (2 M, 4.80 mL) was added and the reaction stirred at 0° C. for 1 h. HCl (4 M, 2.40 mL) was added and the reaction allowed to warm to rt and stirred for 1 h. The reaction was quenched with saturated aq.NaHCO₃ (30 mL) and extracted with EtOAc (50 mL×3). The combined organic layers were washed with brine (20 mL), dried over Na₂SO₄, filtered and the filtrate evaporated under reduced pressure to give trans 2-chloro-1-(2-fluorocyclopropyl)ethan-1-one (170 mg, crude) as a yellow oil. ¹H NMR: (400 MHz, CHLOROFORM-d) 6 μm: 1.49-1.58 (m, 1H), 1.58-1.68 (m, 1H), 2.66-2.75 (m, 1H), 4.25 (s, 2H), 4.70-4.89 (m, 1H).

Step b: To a solution of trans 2-chloro-1-(2-fluorocyclopropyl)ethan-1-one (170 mg, 1.24 mmol) in EtOH (1 mL) was added NaHCO₃ (209.18 mg, 2.49 mmol), Intermediate 3c: N-(6-amino-4-ethoxypyridin-3-yl)-6-(difluoromethyl)picolinamide (170 mg, 551.45 μmol) and KI (20.67 mg, 124.50 μmol) and the reaction stirred at 80° C. for 14 h. The reaction was cooled, filtered and the filtrate concentrated in vacuo. The residue was purified by prep-HPLC (Column: Phenomenex Synergi C18 150×30 mm×4 μm, water (0.05% HCl)-MeCN, from 27% to 47%, to give trans 6-(difluoromethyl)-N-(7-ethoxy-2-(2-fluorocyclopropyl)imidazo[1,2-a]pyridin-6-yl)picolinamide (30 mg, 6.10% yield) as a brown solid. LCMS (ESI) m/z 391.0 (M+H)⁺; The product was further purified by prep-SFC (Column: DAICEL CHIRALCEL OJ-H (250 mm×30 mm, 5 um) eluting with 0.1% NH₄OH EtOH from 0% to 15% to give diastereoisomer 1 (7.30 mg, 24.24% yield) as a white solid. Rf=2.001 min. LCMS (ESI) m/z 391.0 (M+H)⁺; ¹H NMR: (500 MHz, METHANOL-d₄) δ ppm: 1.26-1.32 (m, 1H), 1.60-1.70 (m, 4H), 2.50-2.57 (m, 1H), 4.36-4.41 (m, 2H), 4.91-4.94 (m, 1H), 6.74-6.97 (m, 1H), 7.11 (s, 1H), 7.70 (s, 1H), 7.96 (d, J=8.0 Hz, 1H), 8.24-8.28 (m, 1H), 8.38 (d, J=8.0 Hz, 1H), 9.62 (s, 1H) and diastereoisomer 2 (6.50 mg, 21.69% yield) as a white solid.

¹H NMR: (500 MHz, METHANOL-d₄) δ ppm: 1.21-1.29 (m, 1H), 1.51-1.59 (m, 1H), 1.61 (t, J=7.0 Hz, 3H), 2.43-2.51 (m, 1H), 4.28-4.33 (m, 2H), 4.85-4.88 (m, 1H), 6.72-6.95 (m, 2H), 7.57 (s, 1H), 7.93 (d, J=7.5 Hz, 1H), 8.22-8.26 (m, 1H), 8.35 (d, J=7.5 Hz, 1H), 9.48 (s, 1H).

### Example 255: N-(7-ethoxy-2-((tetrahydrofuran-3-yl)methyl)imidazo[1,2-a]pyridin-6-yl)-2-(1H-pyrazol-4-yl)oxazole-4-carboxamide

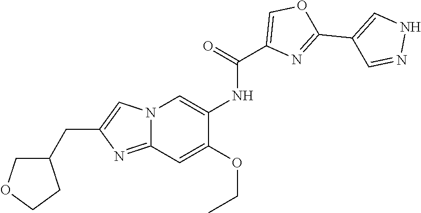 [SMILES: CCOc1cc2nc(CC3CCOC3)cn2cc1NC(=O)c1coc(-c2cn[nH]c2)n1]

N-(7-Ethoxy-2-((tetrahydrofuran-3-yl)methyl)imidazo[1,2-a]pyridin-6-yl)-2-(1H-pyrazol-4-yl)oxazole-4-carboxamide was prepared from 4-(4,4,5,5-tetramethyl-1,3,2-dioxaborolan-2-yl)-1H-pyrazole and Intermediate 3c: N-(6-amino-4-ethoxypyridin-3-yl)-6-(difluoromethyl)picolinamide hydrochloride following the method described in Example 22.

LCMS (ESI) m/z 423.0 (M+H)⁺; ¹H NMR (500 MHz, DMSO-d₆): δ ppm 1.62 (t, J=6.9 Hz, 3H), 1.74-1.65 (m, 1H), 2.01-2.12 (m, 1H), 2.71-2.80 (m, 1H), 2.81 (d, J=7.4 Hz, 2H), 3.50-3.59 (m, 1H), 3.71-3.82 (m, 1H), 3.85-3.96 (m, 2H), 4.23 (q, J=6.9 Hz, 2H), 6.90 (s, 1H), 7.23 (s, 1H), 8.15 (s, 2H), 8.25 (s, 1H), 9.35 (s, 1H), 9.42 (s, 1H).

### Example 256: 6-(difluoromethyl)-N-(7-ethoxy-2-(3-methoxycyclohexyl)imidazo[1,2-a]pyridin-6-yl)picolinamide

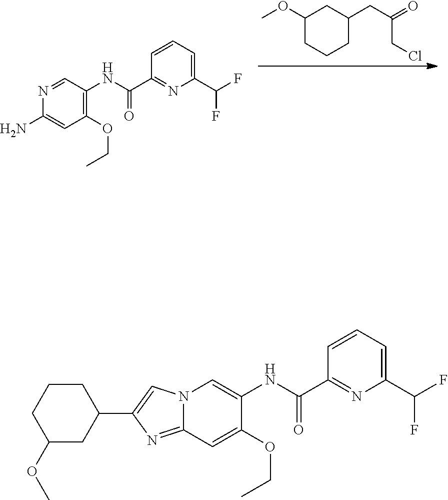 [SMILES: CCOc1cc(N)ncc1NC(=O)c1cccc(C(F)F)n1 ; COC1CCCC(CC(=O)CCl)C1 ; CCOc1cc2nc(C3CCCC(OC)C3)cn2cc1NC(=O)c1cccc(C(F)F)n1]

6-(difluoromethyl)-N-(7-ethoxy-2-(3-methoxycyclohexyl)imidazo[1,2-a]pyridin-6-yl)picolinamide was prepared from 4-methoxycyclohexane-1-carboxylic acid and Intermediate 3c: N-(6-amino-4-ethoxypyridin-3-yl)-6-(difluoromethyl)picolinamide hydrochloride, following a similar synthetic sequence to that described in Example 121. LCMS (ESI) m/z 445.1 (M+H)⁺; ¹H NMR (500 MHz, METHANOL-d₄) δ ppm: 1.34-1.55 (m, 4H), 1.64 (t, J=6.94 Hz, 3H), 1.95-1.99 (m, 1H), 2.00-2.10 (m, 1H), 2.17-2.20 (m, 1H), 2.46-2.49 (m, 1H), 2.83-2.89 (m, 1H), 3.34-3.37 (m, 1H), 3.41 (s, 3H), 4.38 (q, J=6.97 Hz, 2H), 6.96-6.73 (m, 1H), 7.10 (s, 1H), 7.70 (s, 1H), 7.95 (d, J=7.78 Hz, 1H), 8.25 (t, J=7.78 Hz, 1H), 8.37 (d, J=7.78 Hz, 1H), 9.62 (s, 1H)

### Example 257: 6-(difluoromethyl)-N-(2-((1,1-dioxidotetrahydrothiophen-3-yl)methyl)-7-ethoxyimidazo[1,2-a]pyridin-6-yl)picolinamide

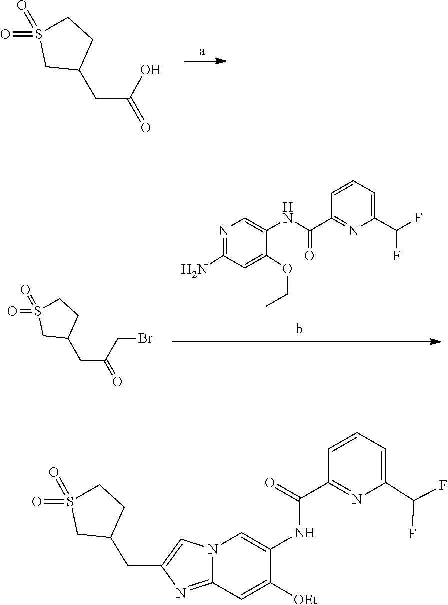 [SMILES: O=C(O)CC1CCS(=O)(=O)C1 ; O=C(CBr)CC1CCS(=O)(=O)C1 ; CCOc1cc(N)ncc1NC(=O)c1cccc(C(F)F)n1 ; CCOc1cc2nc(CC3CCS(=O)(=O)C3)cn2cc1NC(=O)c1cccc(C(F)F)n1]

Step a: DIPEA (739.72 mg, 5.72 mmol) and isobutyl carbonochloridate (689.76 mg, 5.05 mmol) were added drop wise to a mixture of 2-(1,1-dioxidotetrahydrothiophen-3-yl)acetic acid (600 mg, 3.37 mmol) in THF (5 mL) at 0° C. and the reaction stirred for 3 h. Diazomethyl(trimethyl)silane (2 M, 3.37 mL) was added dropwise at 0° C. and the reaction stirred at 0° C. for 1 h and at rt for a further for 14 h. The reaction mixture was cooled to 0° C., HBr (2.84 g, 16.83 mmol, 48% purity) added and the reaction stirred at 0° C. for 1 h. The reaction was quenched with aq. saturated NaHCO₃, the mixture extracted with EtOAc (20 mL×3) and the combined organic phases were washed with brine (50 mL), dried over Na₂SO₄ and filtered. The filtrate was evaporated under reduced pressure to give 1-bromo-3-(1,1-dioxidotetrahydrothiophen-3-yl)propan-2-one (210 mg, 24.45% yield) which was used without purification.

Step b: To a solution of Intermediate 3c: N-(6-amino-4-ethoxypyridin-3-yl)-6-(difluoromethyl)picolinamide (20 mg, 0.065 mmol) and 1-bromo-3-(1,1-dioxidotetrahydrothiophen-3-yl)propan-2-one (100 mg, 0.392 mmol) in EtOH (5 mL) was added NaHCO₃ (16.35 mg, 0.195 mmol). And the reaction stirred at 80° C. for 16 h. The mixture was filtered, and the filtrate concentrated in vacuo. The residue was purified by formic acid mediated prep-HPLC to give 6-(difluoromethyl)-N-(2-((1,1-dioxidotetrahydrothiophen-3-yl)methyl)-7-ethoxyimidazo[1,2-a]pyridin-6-yl)picolinamide (1 mg, 2.15 μmol, 20.01% yield) as a brown solid. LCMS (ESI) m/z 465.0 (M+H)⁺; ¹H NMR (400 MHz, METHANOL-d₄) δ ppm: 1.61 (t, J=7.2 Hz, 3H), 1.99-2.04 (m, 1H), 2.35-2.38 (m, 1H), 2.85-2.91 (m, 4H), 3.19-3.23 (m, 1H), 3.25-3.31 (m, 2H), 4.29 (q, J=6.8 Hz, 2H), 6.70-6.99 (m, 2H), 7.57 (s, 1H), 7.94 (d, J=7.6 Hz, 1H), 8.25 (t, J=8.0 Hz, 1H), 8.37 (d, J=8.0 Hz, 1H), 9.47 (s, 1H),

### Intermediate 19a: 6-(difluoromethyl)-N-(7-isopropoxyimidazo[1,2-a]pyridin-6-yl)picolinamide

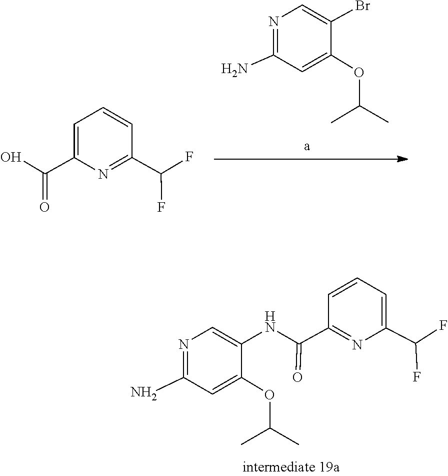 [SMILES: O=C(O)c1cccc(C(F)F)n1 ; CC(C)Oc1cc(N)ncc1Br ; CC(C)Oc1cc(N)ncc1NC(=O)c1cccc(C(F)F)n1]

Step a: DMF (1 drop) followed by oxalyl chloride (1.61 g, 12.71 mmol) were added to a solution of 6-(difluoromethyl)picolinic acid (2.0 g, 11.55 mmol) in benzene (20 mL) and the reaction was stirred at rt overnight. The reaction was evaporated under reduced pressure to give 6-(difluoromethyl)picolinyl chloride. A solution of 4-isopropoxypyridine-2,5-diamine (1.0 g, 5.98 mmol) and TEA (1.21 g, 11.96 mmol) in DCM (30 mL) was cooled to −40° C., a solution of 6-(difluoromethyl)picolinyl chloride (1.20 g, 6.28 mmol) in DCM (5 mL) was added and the reaction was stirred for 2 h at rt. The mixture was washed with water (20 mL) and brine (20 mL), dried over Na₂SO₄ and evaporated under reduced pressure to give Intermediate 19a N-(6-amino-4-isopropoxypyridin-3-yl)-6-(difluoromethyl)picolinamide (1.50 g, 77.82% yield) which was used in the next step without additional purification. ¹H NMR (400 MHz, CHLOROFORM-d) δ ppm 1.42 (d, J=6.1 Hz, 6H), 4.38 (br s, 2H), 4.61-4.63 (m, 1H), 6.05 (br s, 1H), 6.63 (t, J=55.4 Hz, 1H), 7.76 (d, J=7.9 Hz, 1H), 8.04 (t, J=7.8 Hz, 1H), 8.35 (d, J=7.9 Hz, 1H), 9.12 (s, 1H), 10.15 (s, 1H).

Step b: 2-Chloroacetaldehyde (150.25 mg, 957.01 μmol) was added to a solution of N-(6-amino-4-isopropoxypyridin-3-yl)-6-(difluoromethyl)picolinamide (118.64 mg, 368.08 μmol) in MeCN (2.38 mL) and the reaction stirred under reflux for 1 h. The cooled mixture was acidified with HCl 4N dioxane (0.1 mL) then concentrated in vacuo. The residue was suspended in MeOH, water (1 drop) and the mixture purified by ion exchange chromatography on Hypersep™ SCX (resin bound sulfonate) eluting with MeOH to 2N methanolic ammonia to give Intermediate 19b 6-(difluoromethyl)-N-(7-isopropoxyimidazo[1,2-a]pyridin-6-yl)picolinamide. LCMS (ESI) m/z 347.0 (M+H)⁺; ¹H NMR (500 MHz, CHLOROFORM-d) δ ppm: 1.46 (d, J=6.10 Hz, 6H), 4.96-5.07 (m, 1H), 7.01-7.28 (m, 1H), 7.36 (br s, 1H), 7.75 (s, 1H), 8.04 (d, J=6.71 Hz, 1H), 8.16 (br s, 1H), 8.29-8.41 (m, 2H), 9.72 (s, 1H), 9.69-9.73 (m, 1H), 10.61 (s, 1H).

Example 258: 6-(difluoromethyl)-N-(2-(3-fluorobicyclo[1.1.1]pentan-1-yl)-7-isopropoxyimidazo[1,2-a]pyridin-6-yl)picolinamide was prepared by the standard condensation reaction conditions as in Example 55 with A mixture of 2-chloro-1-(3-fluorobicyclo[1.1.1]pentan-1-yl)ethan-1-one (250 mg, 1.54 mmol), and Intermediate 19a: to give 6-(difluoromethyl)-N-(2-(3-fluorobicyclo[1.1.1]pentan-1-yl)-7-isopropoxyimidazo[1,2-a]pyridin-6-yl)picolinamide (110 mg, 18.17% yield). LCMS (ESI) m/z 431.2 (M+H); ¹H NMR (400 MHz, DMSO-d₆) δ ppm 1.41 (d, J=6.0 Hz, 6H), 2.34 (d, J=2.6 Hz, 6H), 4.83-4.85 (m, 1H), 6.93-7.31 (m, 2H), 7.75 (s, 1H), 7.96-8.05 (m, 1H), 8.24-8.36 (m, 2H), 9.43 (s, 1H), 10.50 (s, 1H)

Example 259: 6-(difluoromethyl)-N-(7-isopropoxy-2-(1-methyl-2-oxabicyclo[3.1.1]heptan-5-yl)imidazo[1,2-a]pyridin-6-yl)picolinamide was prepared by the standard condensation reaction conditions as in Example 55 with A mixture of intermediate 19a: N-(6-amino-4-isopropoxypyridin-3-yl)-6-(difluoromethyl)picolinamide (15.0 mg, 46.54 μmol), and 2-bromo-1-(1-methyl-2-oxabicyclo[3.1.1]heptan-5-yl)ethanone (16.50 mg, 47.47 μmol) to give 6-(difluoromethyl)-N-(7-isopropoxy-2-(1-methyl-2-oxabicyclo[3.1.1]heptan-5-yl)imidazo[1,2-a]pyridin-6-yl)picolinamide as an off-white solid. 19.20 mg. LCMS (ESI) m/z 457.3 (M+H)⁺; ¹H NMR (500 MHz, CHLOROFORM-d) δ ppm 1.35 (s, 3H), 1.52 (d, J=6.10 Hz, 6H), 2.27 (s, 4H), 2.39 (t, J=6.87 Hz, 2H), 4.23 (t, J=6.87 Hz, 2H), 4.64-4.78 (m, 1H), 6.50-6.83 (m, 1H), 7.01 (s, 1H), 7.19 (s, 1H), 7.84 (d, J=7.78 Hz, 1H), 8.11 (t, J=7.78 Hz, 1H), 8.39 (d, J=7.78 Hz, 1H), 9.44 (s, 1H), 10.57 (s, 1H)

Example 260: N-(2-(2-oxabicyclo[2.2.1]heptan-4-yl)-7-isopropoxyimidazo[1,2-a]pyridin-6-yl)-6-(difluoromethyl)picolinamide was prepared by the standard condensation reaction conditions as in Example 55 with A mixture of 2-bromo-1-(2-oxabicyclo[2.2.1]heptan-4-yl)ethanone (25.94 mg, 118.4 μmol), and intermediate 19a: N-(6-amino-4-isopropoxypyridin-3-yl)-6-(difluoromethyl)picolinamide (36 mg, 111.7 μmol) to give N-(2-(2-oxabicyclo[2.2.1]heptan-4-yl)-7-isopropoxyimidazo[1,2-a]pyridin-6-yl)-6-(difluoromethyl)picolinamide, 42 mg as a pale yellow solid. LCMS (ESI) m/z 443.3 (M+H)⁺; ¹H NMR (400 MHz, CHLOROFORM-d) δ ppm 1.51 (d, J=6.02 Hz, 6H), 1.76-2.21 (m, 6H), 3.83-4.05 (m, 2H), 4.61-4.79 (m, 1H), 6.46-6.83 (m, 1H), 6.95 (s, 1H), 7.29 (s, 1H), 7.84 (d, J=7.03 Hz, 1H), 8.11 (t, J=7.78 Hz, 1H), 8.38 (d, J=7.78 Hz, 1H), 9.44 (s, 1H), 10.56 (s, 1H).

Example 261: methyl 2-(6-(6-(difluoromethyl)picolinamido)-7-isopropoxyimidazo[1,2-a]pyridin-2-yl)-2-methylpropanoate was prepared by the standard condensation reaction conditions as in Example 55 with A mixture of intermediate a: N-(6-amino-4-isopropoxypyridin-3-yl)-6-(difluoromethyl)picolinamide (57 mg, 176.85 μmol), and methyl 4-bromo-2,2-dimethyl-3-oxo-butanoate to give methyl 2-(6-(6-(difluoromethyl)picolinamido)-7-isopropoxyimidazo[1,2-a]pyridin-2-yl)-2-methylpropanoate as a white solid, 15 mg. LCMS (ESI) m/z 447.4 (M+H)⁺; 1H NMR (400 MHz, METHANOL-d₄) δ ppm: 1.52 (d, J=6.02 Hz, 6H), 1.63 (s, 6H), 3.67 (s, 3H), 4.87-4.90 (m, 1H), 6.68-7.01 (m, 2H), 7.63 (d, J=0.75 Hz, 1H), 7.93 (d, J=7.03 Hz, 1H), 8.24 (t, J=7.65 Hz, 1H), 8.37 (dd, J=0.75, 7.78 Hz, 1H), 9.49 (d, J=0.75 Hz, 1H).

Example 262: 2-(6-(6-(difluoromethyl)picolinamido)-7-isopropoxyimidazo[1,2-a]pyridin-2-yl)-2-methylpropanoic acid was prepared by standard ester hydrolysis of example 261 to yield 2-(6-(6-(difluoromethyl)picolinamido)-7-isopropoxyimidazo[1,2-a]pyridin-2-yl)-2-methylpropanoic acid as a white solid, 9 mg. LCMS (ESI) m/z 433.3 (M+H)⁺; ¹H NMR (400 MHz, METHANOL-d₄) δ ppm: 1.57 (d, J=6.02 Hz, 6H), 1.65 (s, 6H), 4.94-5.06 (m, 1H), 6.68-7.03 (m, 1H), 7.11 (s, 1H), 7.76 (s, 1H), 7.96 (d, J=7.28 Hz, 1H), 8.27 (t, J=7.78 Hz, 1H), 8.40 (dd, J=1.00, 7.78 Hz, 1H), 9.65 (s, 1H).

Example 263: 6-(difluoromethyl)-N-(2-(1,3-dimethyl-1H-pyrazol-4-yl)-7-isopropoxyimidazo[1,2-a]pyridin-6-yl)picolinamide was prepared by the standard condensation reaction conditions as in Example 55 with A mixture of intermediate 19a: N-(6-amino-4-isopropoxypyridin-3-yl)-6-(difluoromethyl)picolinamide (80 mg, 248.21 μmol) and 2-bromo-1-(1,3-dimethylpyrazol-4-yl)ethan-1-one (53.88 mg, 248.21 μmol) to obtain 6-(difluoromethyl)-N-(2-(1,3-dimethyl-1H-pyrazol-4-yl)-7-isopropoxyimidazo[1,2-a]pyridin-6-yl)picolinamide (26.80 mg, 24.51% yield). LCMS (ESI) m/z 441.3 (M+H)⁺; ¹H NMR (500 MHz, DMSO-d₆) δ ppm 1.47 (d, J=5.49 Hz, 6H), 1.47-1.51 (m, 1H), 2.35-2.40 (m, 4H), 3.82-3.87 (m, 3H), 4.99-5.09 (m, 1H), 7.04-7.29 (m, 2H), 8.01-8.06 (m, 2H), 8.21 (br s, 1H), 8.33-8.39 (m, 2H), 9.66 (br s, 1H), 10.61 (s, 1H)

Example 264: 6-(difluoromethyl)-N-(2-(3-(difluoromethyl)-1-methyl-1H-pyrazol-4-yl)-7-isopropoxyimidazo[1,2-a]pyridin-6-yl)picolinamide was prepared by the standard condensation reaction conditions as in Example 55 with A mixture of intermediate 19a: N-(6-amino-4-isopropoxypyridin-3-yl)-6-(difluoromethyl)picolinamide (80 mg, 248.21 μmol) and 2-chloro-1-(3-(difluoromethyl)-1-methyl-1H-pyrazol-4-yl)ethan-1-one (77.66 mg, 372.32 μmol) to obtain 6-(difluoromethyl)-N-(2-(3-(difluoromethyl)-1-methyl-1H-pyrazol-4-yl)-7-isopropoxyimidazo[1,2-a]pyridin-6-yl)picolinamide (26.40 mg, 22.32% yield). LCMS (ESI) m/z 477.3 (M+H)⁺; 1H NMR (500 MHz, DMSO-d₆) δ ppm 1.48 (d, J=6.10 Hz, 7H), 3.94-4.03 (m, 3H), 5.09 (br s, 1H), 7.02-7.42 (m, 3H), 8.05 (dd, J=7.32, 1.22 Hz, 1H), 8.28-8.44 (m, 3H), 9.80 (br s, 1H), 10.63 (s, 1H).

Example 265: 6-(difluoromethyl)-N-(7-isopropoxy-2-((tetrahydrofuran-3-yl)methyl)imidazo[1,2-a]pyridin-6-yl)picolinamide was prepared by the standard condensation reaction conditions as in Example 55 with a solution of intermediate 19a: N-(6-amino-4-isopropoxypyridin-3-yl)-6-(difluoromethyl)picolinamide (150 mg, 465.39 μmol) and 1-bromo-3-(tetrahydrofuran-3-yl)propan-2-one (104.74 mg, 465.39 μmol) to give 6-(difluoromethyl)-N-(7-isopropoxy-2-((tetrahydrofuran-3-yl)methyl)imidazo[1,2-a]pyridin-6-yl)picolinamide (58.90 mg, 29.40% yield). LCMS (ESI) m/z 431.2 (M+H)⁺; ¹H NMR (400 MHz, DMSO-d₆): δ 1.41 (d, J=5.9 Hz, 6H), 1.55-1.66 (m, 1H), 1.94-2.04 (m, 1H), 2.53-2.61 (m, 1H), 2.64-2.70 (m, 2H), 3.38-3.43 (m, 1H), 3.60-3.68 (m, 1H), 3.71-3.81 (m, 2H), 4.83-4.91 (m, 1H), 6.94-7.31 (m, 2H), 7.68 (s, 1H), 8.00 (dd, J=6.0, 2.9 Hz, 1H), 8.27-8.36 (m, 2H), 9.42 (s, 1H), 10.50 (s, 1H). 1-bromo-3-(tetrahydrofuran-3-yl)propan-2-one was prepared as follows: To a solution of 2-(tetrahydrofuran-3-yl)acetic acid (2.0 g, 15.37 mmol) in DCM (50 mL) was added oxalyl chloride (3.90 g, 30.74 mmol) at 0° C. under Ar(g) and the reaction stirred at 30° C. for 16 h. The mixture was concentrated in vacuo and the residue co-evaporated with anhydrous DCM (20 mL×3) to give 2-(tetrahydrofuran-3-yl)acetyl chloride (2.50 g) as a yellowish oil. This was dissolved in THF (100 mL) and diazomethane (1 M solution in MTBE, 67.28 mL) added dropwise at 5° C.

The solution was stirred at this temperature for 0.5 h, then cooled to 0° C. and aqueous HBr (7 M, 4.81 mL) was added dropwise. The resulting mixture was stirred at rt for 40 min, then diluted with saturated NaHCO₃ aqueous solution (100 mL), the layers separated and the aqueous layer extracted with MTBE (50 mL). The combined organic phases were washed with brine (20 mL), dried over Na₂SO₄, filtered and concentrated in vacuo to give 1-bromo-3-(tetrahydrofuran-3-yl)propan-2-one as a yellow liquid, 3.2 g, which was used in the next step without further purification. ¹H NMR (400 MHz, CDCl₃): δ 1.42-1.53 (m, 1H), 2.06-2.16 (m, 1H), 2.57-2.67 (m, 1H), 2.68-2.86 (m, 2H), 3.29-3.37 (m, 1H), 3.68-3.75 (m, 1H), 3.77-3.87 (m, 3H), 3.87-3.95 (m, 1H).

Step b: A solution of intermediate a: N-(6-amino-4-isopropoxypyridin-3-yl)-6-(difluoromethyl)picolinamide (150 mg, 465.39 μmol) and 1-bromo-3-(tetrahydrofuran-3-yl)propan-2-one (104.74 mg, 465.39 μmol) in EtOH (10 mL) was heated under reflux for 48 h. The solvent was evaporated under reduced pressure, H₂O (10 mL) and NaHCO₃ (78.19 mg, 930.78 μmol) were added, and the product extracted with CHCl₃ (3×10 mL). The combined organic phases were dried over Na₂SO₄, filtered and evaporated under reduced pressure. The crude product was purified by prep HPLC (YMC-Actus Triart C18 100×20 mm, Sum) eluting with water/MeOH+0.1% vol. 0.25 aq. NH₃ from 55 to 90%) to give 6-(difluoromethyl)-N-(7-isopropoxy-2-((tetrahydrofuran-3-yl)methyl)imidazo[1,2-a]pyridin-6-yl)picolinamide (58.90 mg, 29.40% yield). LCMS (ESI) m/z 431.2 (M+H)⁺; ¹H NMR (400 MHz, DMSO-d₆): δ 1.41 (d, J=5.9 Hz, 6H), 1.55-1.66 (m, 1H), 1.94-2.04 (m, 1H), 2.53-2.61 (m, 1H), 2.64-2.70 (m, 2H), 3.38-3.43 (m, 1H), 3.60-3.68 (m, 1H), 3.71-3.81 (m, 2H), 4.83-4.91 (m, 1H), 6.94-7.31 (m, 2H), 7.68 (s, 1H), 8.00 (dd, J=6.0, 2.9 Hz, 1H), 8.27-8.36 (m, 2H), 9.42 (s, 1H), 10.50 (s, 1H).

### Example 266: N-(7-isopropoxy-2-tetrahydropyran-4-yl-imidazo[1,2-a]pyridin-6-yl)-6-(trifluoromethyl)picolinamide

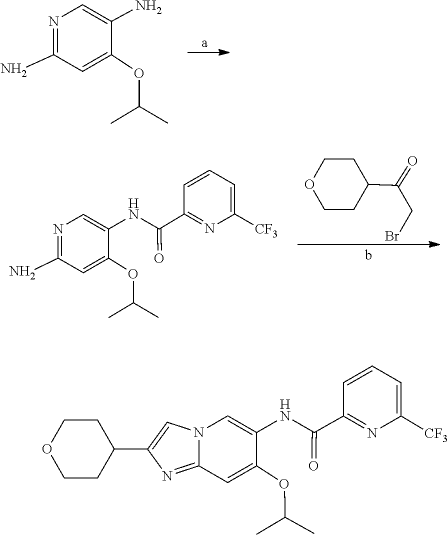 [SMILES: CC(C)Oc1cc(N)ncc1N ; CC(C)Oc1cc(N)ncc1NC(=O)c1cccc(C(F)(F)F)n1 ; O=C(CBr)C1CCOCC1 ; CC(C)Oc1cc2nc(C3CCOCC3)cn2cc1NC(=O)c1cccc(C(F)(F)F)n1]

Step a: DIPEA (2.32 g, 17.94 mmol) was added to a mixture of 4-isopropoxypyridine-2,5-diamine (1.00 g, 5.98 mmol), HATU (2.28 g, 5.98 mmol) and 6-(trifluoromethyl)picolinic acid (1.14 g, 5.98 mmol) in DMF (9.06 mL) at 0° C. and the reaction stirred at rt overnight. The reaction was quenched with brine, extracted with EtOAc (3×), the combined organic extracts washed with brine, dried over MgSO₄, filtered and concentrated in vacuo. The crude was purified by silica gel column chromatography (dry loaded, 0-100% 3:1 EtOAc:EtOH+1% TEA in heptanes) to obtain N-(6-amino-4-isopropoxy-3-pyridyl)-6-(trifluoromethyl)picolinamide as a yellow solid.

Step b: A mixture of N-(6-amino-4-isopropoxy-3-pyridyl)-6-(trifluoromethyl)picolinamide (100 mg, 293.86 μmol), 2-bromo-1-tetrahydropyran-4-yl-ethanone (30.42 mg, 146.93 μmol) and NaHCO₃ (74.06 mg, 881.58 μmol) in MeCN (705.26 μL) and toluene (470.18 μL) was heated at 90° C. overnight. The reaction was cooled to rt, filtered and the filtrate evaporated under reduced pressure. The residue was purified by TFA modified mass directed prep. HPLC to give N-(7-isopropoxy-2-tetrahydropyran-4-yl-imidazo[1,2-a]pyridin-6-yl)-6-(trifluoromethyl)picolinamide (25.90 mg, 19.65% yield) LCMS (ESI) m/z 449.4 (M+H)⁺; ¹H NMR (500 MHz, DMSO-d₆) δ ppm 1.47 (d, J=6.10 Hz, 6H), 1.63-1.79 (m, 2H), 1.96 (br dd, J=12.82, 1.83 Hz, 2H), 3.06-3.19 (m, 1H), 3.46-3.54 (m, 1H), 3.91-4.01 (m, 2H), 5.07-5.16 (m, 1H), 7.39 (s, 1H), 8.14 (s, 1H), 8.30 (dd, J=7.32, 1.22 Hz, 1H), 8.44-8.53 (m, 2H), 9.76 (s, 1H), 10.56 (s, 1H)

### Example 267: 1-(difluoromethyl)-N-(2-(difluoromethyl)-7-isopropoxyimidazo[1,2-a]pyridin-6-yl)-1H-pyrazole-3-carboxamide

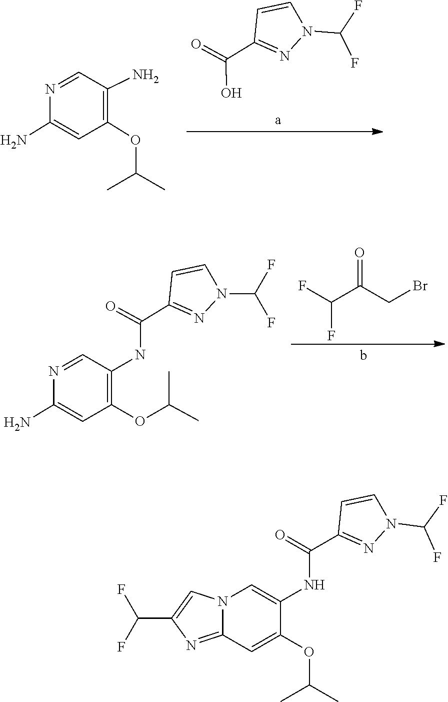

Step a: To a solution of 1-(difluoromethyl)-1H-pyrazole-3-carboxylic acid (1.00 g, 6.17 mmol) in DCM (20 mL) was added oxalyl chloride (1.57 g, 12.34 mmol) at 0° C. under Ar(g) and the reaction stirred at 30° C. for 16 h. The mixture was concentrated in vacuo and the residue azeotroped with anhydrous DCM (10 mL×3) to give 1-(difluoromethyl)-1H-pyrazole-3-carbonyl chloride as a yellowish oil. A solution of 4-isopropoxypyridine-2,5-diamine (1.03 g, 6.16 mmol) and TEA (1.23 g, 12.2 mmol) in DCM (50 mL) was cooled to −40° C. and a solution of 1-(difluoromethyl)-1H-pyrazole-3-carbonyl chloride (1.11 g, 6.16 mmol) in DCM (10 mL) added dropwise. The resulting mixture was stirred at −40° C. for 1 h and then at rt overnight. The reaction mixture was washed with brine, dried over Na₂SO₄, filtered and concentrated in vacuo to give N-(6-amino-4-isopropoxypyridin-3-yl)-1-(difluoromethyl)-1H-pyrazole-3-carboxamide (1.90 g, 99% yield) as a dark-brown solid. LCMS (ESI) m/z 312.2 (M+H)⁺;

Step b: A mixture of N-(6-amino-4-isopropoxypyridin-3-yl)-1-(difluoromethyl)-1H-pyrazole-3-carboxamide (200 mg, 578.24 μmol) and 3-bromo-1,1-difluoropropan-2-one (150.02 mg, 607.15 μmol) in EtOH (10 mL) was heated under reflux for 48 h. The solvent was evaporated in vacuo, H₂O (10 mL) and NaHCO₃ (97.16 mg, 1.16 mmol) were added, and the mixture was extracted with CHCl₃ (3×10 mL). The combined organic phases were dried over Na₂SO₄, filtered and evaporated under reduced pressure. The crude was purified by TFA modified mass directed prep. HPLC to give 1-(difluoromethyl)-N-(2-(difluoromethyl)-7-isopropoxyimidazo[1,2-a]pyridin-6-yl)-1H-pyrazole-3-carboxamide (69.1 mg, 31.0% yield). LCMS (ESI) m/z 386.2 (M+H)⁺; ¹H NMR (400 MHz, DMSO-d₆): δ 1.39 (d, J=6.0 Hz, 6H), 4.84-4.93 (m, 1H), 6.89-7.23 (m, 3H), 7.96 (t, J=58.6 Hz, 1H), 8.20 (s, 1H), 8.47 (d, J=2.7 Hz, 1H), 9.35 (s, 1H), 9.41 (s, 1H).

### Intermediate 19: N-(6-amino-5-methoxypyridin-3-yl)-6-(difluoromethyl)picolinamide

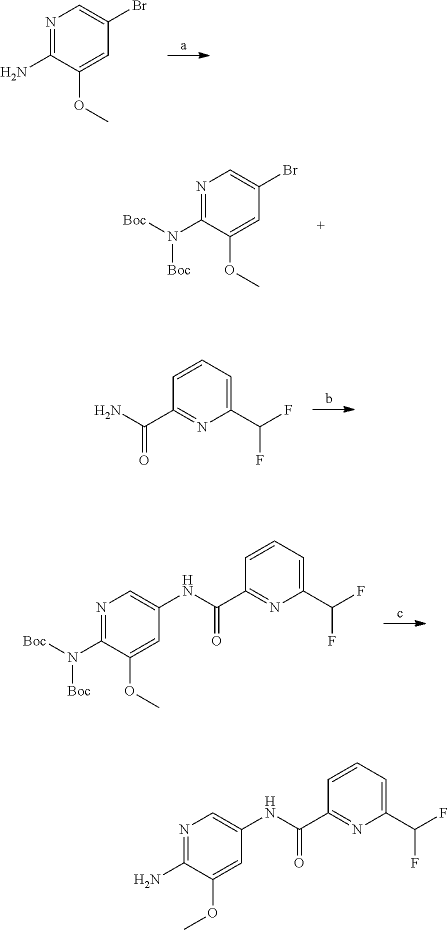

Step a: To a solution of 5-bromo-3-methoxypyridin-2-amine (3.00 g, 14.78 mmol) and Boc₂O (12.9 g, 59.2 mmol) in DCM (59.12 mL) were added DMAP (1.81 g, 14.78 mmol) and TEA (4.49 g, 44.34 mmol, 6.15 mL) and the reaction was stirred at 25° C. overnight. The cooled mixture was concentrated in vacuo and purified by column chromatography on silica gel to give tert-butyl (5-bromo-3-methoxypyridin-2-yl)(tert-butoxycarbonyl)carbamate. LCMS (ESI) m/z 403.1 (M+H)⁺;

Step b: A mixture of tert-butyl (5-bromo-3-methoxypyridin-2-yl)(tert-butoxycarbonyl)carbamate (3.90 g, 9.67 mmol), Pd(OAc)₂ (434.24 mg, 1.93 mmol), Xantphos (2.24 g, 3.87 mmol), Cs₂CO₃ (6.30 g, 19.34 mmol) and 6-(difluoromethyl)picolinamide (4.16 g, 24.17 mmol) was purged with N₂. Dioxane (96.70 mL) was added at rt, the reaction vessel sealed and heated at 80° C. overnight. The cooled reaction mixture was filtered through Celite® and the filtrate concentrated in vacuo. The residue was purified by column chromatography to give tert-butyl (tert-butoxycarbonyl)(5-(6-(difluoromethyl)picolinamido)-3-methoxypyridin-2-yl)carbamate, which contained some impurities. LCMS (ESI) m/z 495.2 (M+H)⁺;

Step c: TFA (14.75 g, 129.40 mmol) was added to a solution of tert-butyl (tert-butoxycarbonyl)(5-(6-(difluoromethyl)picolinamido)-3-methoxypyridin-2-yl)carbamate (6.40 g, 12.94 mmol) in DCM (32.35 mL) and the reaction stirred at rt. When LCMS indicated that the reaction was complete, the mixture was evaporated under reduced pressure was to give N-(6-amino-5-methoxy-3-pyridyl)-6-(difluoromethyl)pyridine-2-carboxamide 2,2,2-trifluoroacetate. LCMS (ESI) m/z 295.0 (M+H)⁺;

### Example 268, 269 and 270: 6-(difluoromethyl)-N-(8-methoxy-2-(tetrahydro-2H-pyran-3-yl)imidazo[1,2-a]pyridin-6-yl)picolinamide

Enantiomer 1 and 2 were prepared by standard condensation reaction conditions with

A mixture of Intermediate 19: N-(6-amino-5-methoxy-3-pyridyl)-6-(difluoromethyl)pyridine-2-carboxamide 2,2,2-trifluoroacetate (242.49 mg, 824.06 μmol), and 2-chloro-1-tetrahydropyran-3-yl-ethanone (134 mg, 824.06 μmol). The crude product was purified by reverse phase HPLC to give Example 268: 6-(difluoromethyl)-N-(8-methoxy-2-(tetrahydro-2H-pyran-3-yl)imidazo[1,2-a]pyridin-6-yl)picolinamide. LCMS (ESI) m/z 403.2 (M+H)⁺; ¹H NMR (500 MHz, DMSO-d₆) δ ppm 1.51-1.93 (m, 4H), 2.04-2.21 (m, 1H), 3.42-3.56 (m, 1H), 3.69-3.97 (m, 2H), 4.03 (br dd, J=10.68, 3.36 Hz, 1H), 4.10 (s, 3H), 6.87-7.24 (m, 1H), 7.80 (br s, 1H), 8.05 (br d, J=6.71 Hz, 1H), 8.18-8.43 (m, 2H), 9.35 (s, 1H), 10.89 (br s, 1H).

This compound was further purified by SFC using a CHIRALPAK IB 30×250 mm, 5 μm column, eluting with 30% MeOH w/0.1% DEA in CO₂, flow rate of 100 mL/min, MBPR 40 psi, 40 C column to provide enantiomer 1, Rf=2.58 min.

Further elution provided enantiomer 2, Rf=2.90 min.

Example 271: 6-(difluoromethyl)-N-(8-methoxy-2-((1r*,3r*)-3-methoxy-1-methylcyclobutyl)imidazo[1,2-a]pyridin-6-yl)picolinamide was prepared under standard condensation reaction conditions with Intermediate 19: N-(6-amino-5-methoxy-3-pyridyl)-6-(difluoromethyl)picolinamide (100 mg, 0.340 mmol) and 2-chloro-1-(3-methoxy-1-methylcyclobutyl)ethan-1-one (120 mg, 0.680 mmol) to give 6-(difluoromethyl)-N-(8-methoxy-2-((1r*,3r*)-3-methoxy-1-methylcyclobutyl)imidazo[1,2-a]pyridin-6-yl)picolinamide (10 mg, 7% yield, Rf=1.579 min) as an off-white solid. LCMS (ESI) m/z 417.0 (M+H)⁺; ¹H NMR (500 MHz, METHANOL-d₄) δ ppm: 1.59 (s, 3H), 2.05-2.15 (m, 2H), 2.75-2.85 (m, 2H), 3.25 (s, 3H), 3.90-3.95 (m, 1H), 3.60-3.65 (m, 1H), 4.12 (s, 3H), 6.75-7.00 (m, 1H), 7.38 (s, 1H), 7.92 (s, 1H), 7.96 (d, J=8.0 Hz, 1H), 8.10-8.20 (m, 1H), 8.24 (t, J=8.0 Hz, 1H), 8.37 (d, J=8.0 Hz, 1H), 9.13 (s, 1H).

### Example 272: N-(8-chloro-7-ethoxy-2-(tetrahydro-2H-pyran-4-yl)imidazo[1,2-a]pyridin-6-yl)-6-(difluoromethyl)picolinamide

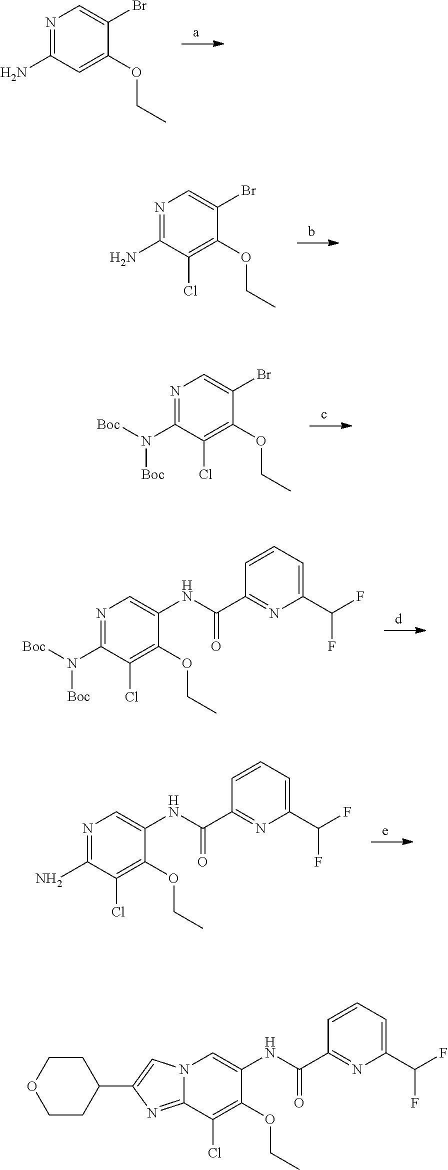 [SMILES: CCOc1cc(N)ncc1Br ; CCOc1c(Br)cnc(N)c1Cl ; CCOc1c(Br)cnc(N(C(=O)OC(C)(C)C)C(=O)OC(C)(C)C)c1Cl ; CCOc1c(NC(=O)c2cccc(C(F)F)n2)cnc(N(C(=O)OC(C)(C)C)C(=O)OC(C)(C)C)c1Cl ; CCOc1c(NC(=O)c2cccc(C(F)F)n2)cnc(N)c1Cl ; CCOc1c(NC(=O)c2cccc(C(F)F)n2)cn2cc(C3CCOCC3)nc2c1Cl]

Step a: NCS (677.13 mg, 5.07 mmol) was added to a solution of 5-bromo-4-ethoxypyridin-2-amine (1.00 g, 4.61 mmol) in DMF (4.61 mL) and the reaction stirred overnight. The solution was treated with sat. aq. NaHCO₃ and brine, then extracted with EtOAc. The combined organic extracts were dried, filtered and evaporated under reduced pressure to give 5-bromo-3-chloro-4-ethoxypyridin-2-amine, which was used without further purification. ¹H NMR (500 MHz, METHANOL-d₄) δ ppm 1.33-1.56 (m, 4H), 4.07-4.25 (m, 2H), 7.91-7.97 (m, 1H).

Step b: To a solution of 5-bromo-3-chloro-4-ethoxypyridin-2-amine (1.24 g, 4.94 mmol) and Boc₂O (4.31 g, 19.76 mmol) in DCM (16.47 mL) were added DMAP (602.92 mg, 4.94 mmol) and TEA (1.50 g, 14.82 mmol) and the reaction was stirred at 25° C. overnight. The mixture was concentrated in vacuo and the residue purified by column chromatography on silica gel to give tert-butyl (5-bromo-3-chloro-4-ethoxypyridin-2-yl)(tert-butoxycarbonyl)carbamate (900 mg, 40.33% yield). ¹H NMR (500 MHz, CDCl₃) δ ppm 0.83-0.99 (m, 5H), 1.43 (s, 13H), 4.22-4.34 (m, 2H), 8.27-8.60 (m, 1H).

Step c: A vial charged with tert-butyl (5-bromo-3-chloro-4-ethoxypyridin-2-yl)(tert-butoxycarbonyl)carbamate (898.96 mg, 1.99 mmol), Xantphos (230.29 mg, 398.00 μmol), Cs₂CO₃ (1.30 g, 3.98 mmol), Pd₂(dba)₃ (182.23 mg, 199 μmol) and 6-(difluoromethyl)picolinamide (342.54 mg, 1.99 mmol) was purged with N₂ and closed with a screw cap with septa. Toluene (19.90 mL) was added, the vial was sealed and heated at 100° C. overnight. Silica was added to the cooled mixture, and the solvent removed in vacuo. The solid was purified by column chromatography on silica gel to give tert-butyl (tert-butoxycarbonyl)(3-chloro-5-(6-(difluoromethyl)picolinamido)-4-ethoxypyridin-2-yl)carbamate (260 mg, 24.06% yield). ¹H NMR (500 MHz, CDCl₃) δ ppm 1.60 (s, 3H), 4.18-4.49 (m, 2H), 6.56-6.93 (m, 1H), 7.90 (d, J=7.94 Hz, 1H), 8.02-8.21 (m, 1H), 8.31-8.50 (m, 1H), 9.60 (s, 1H), 9.46-9.75 (m, 1H).

Step d: tert-Butyl (tert-butoxycarbonyl)(3-chloro-5-(6-(difluoromethyl)picolinamido)-4-ethoxypyridin-2-yl)carbamate (260 mg, 478.86 μmol) was dissolved in DCM (1.60 mL) and TFA (1.09 g, 9.58 mmol) was added. The solution was stirred at rt and then evaporated under reduced pressure to give N-(6-amino-5-chloro-4-ethoxy-3-pyridyl)-6-(difluoromethyl)pyridine-2-carboxamide 2,2,2-trifluoroacetate. LCMS (ESI) m/z 342.9 (M+H)⁺

Step e: A mixture of N-(6-amino-5-chloro-4-ethoxy-3-pyridyl)-6-(difluoromethyl)pyridine-2-carboxamide 2,2,2-trifluoroacetate (116.00 mg, 338.46 μmol), 2-bromo-1-tetrahydropyran-4-yl-ethan-1-one (70.08 mg, 338.46 μmol) and NaHCO₃ (85.30 mg, 1.02 mmol) in EtOH (846.15 μL) was heated at 80° C. overnight. The reaction was cooled to rt, filtered, and the filtrate concentrated in vacuo. The residue was purified via reverse phase HPLC, to give N-(8-chloro-7-ethoxy-2-(tetrahydro-2H-pyran-4-yl)imidazo[1,2-a]pyridin-6-yl)-6-(difluoromethyl)picolinamide (2.20 mg, 4.88 μmol, 1.44% yield). LCMS (ESI) m/z 450.9 (M+H)⁺; ¹H NMR (500 MHz, DMSO-d₆) δ ppm 1.52 (t, J=7.02 Hz, 3H), 1.70 (qd, J=12.31, 3.97 Hz, 2H), 1.94 (br dd, J=12.51, 2.14 Hz, 2H), 3.30-3.40 (m, 1H), 3.44-3.57 (m, 1H), 3.95 (br dd, J=11.29, 2.14 Hz, 2H), 4.25-4.43 (m, 2H), 6.99-7.38 (m, 1H), 8.05 (dd, J=7.02, 1.53 Hz, 1H), 8.26-8.44 (m, 2H), 9.56 (br s, 1H), 10.42 (br s, 1H).

### Intermediate 20: N-(5-aminopyrazin-2-yl)-6-(difluoromethyl)picolinamide

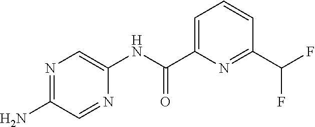 [SMILES: Nc1cnc(NC(=O)c2cccc(C(F)F)n2)cn1]

N-(5-aminopyrazin-2-yl)-6-(difluoromethyl)picolinamide was prepared from 5-bromopyrazin-2-amine and 6-(difluoromethyl)pyridine-2-carboxamide following the method described in Example 272, steps b and c. LCMS (ESI) m/z 266.0 (M+H)⁺;

### Example 273: N-(2-(6-oxaspiro[3.4]octan-2-yl)imidazo[1,2-a]pyrazin-6-yl)-6-(difluoromethyl)picolinamide

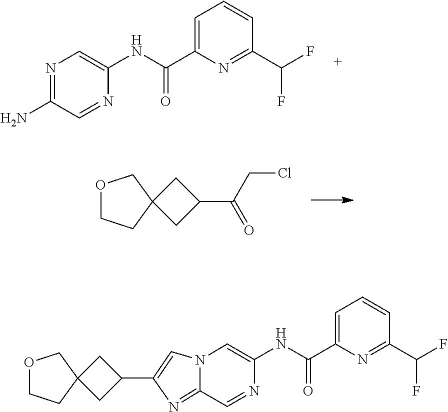 [SMILES: Nc1cnc(NC(=O)c2cccc(C(F)F)n2)cn1 ; O=C(CCl)C1CC2(CCOC2)C1 ; O=C(Nc1cn2cc(C3CC4(CCOC4)C3)nc2cn1)c1cccc(C(F)F)n1]

A mixture of Intermediate 20: N-(5-aminopyrazin-2-yl)-6-(difluoromethyl)picolinamide (100 mg, 377.05 μmol), 2-chloro-1-(6-oxaspiro[3.4]octan-2-yl)ethan-1-one (71.13 mg, 377.05 μmol) in MeCN (754.10 μL) and toluene (189 μL) was heated at 80° C. overnight. The reaction mixture was cooled to rt, filtered, and the filtrate concentrated in vacuo. The crude was purified by TFA modified mass directed prep HPLC, to give N-(2-(6-oxaspiro[3.4]octan-2-yl)imidazo[1,2-a]pyrazin-6-yl)-6-(difluoromethyl)picolinamide 2,2,2-trifluoroacetate (3 mg, 5.85 μmol, 1.55% yield). LCMS (ESI) m/z 400.0 (M+H)⁺; ¹H NMR (500 MHz, DMSO-d₆) δ ppm 2.26-2.39 (m, 4H), 3.60-3.80 (m, 4H), 7.00-7.33 (m, 1H), 8.01 (dd, J=7.32, 1.22 Hz, 1H), 8.19 (d, J=3.05 Hz, 1H), 8.29-8.40 (m, 2H), 8.93 (s, 1H), 9.38 (t, J=1.53 Hz, 1H), 10.33 (s, 1H).

Example 274: 6-(difluoromethyl)-N-(2-(1-methyl-2-oxabicyclo[2.1.1]hexan-4-yl)imidazo[1,2-a]pyrazin-6-yl)picolinamide was prepared under condensation conditions as in Example 273 with A mixture of Intermediate 20: N-(5-aminopyrazin-2-yl)-6-(difluoromethyl)pyridine-2-carboxamide (912.36 mg, 3.44 mmol), and 2-chloro-1-(1-methyl-2-oxabicyclo[2.1.1]hexan-4-yl)ethan-1-one (300.35 mg, 1.72 mmol) to give 6-(difluoromethyl)-N-(2-(1-methyl-2-oxabicyclo[2.1.1]hexan-4-yl)imidazo[1,2-a]pyrazin-6-yl)picolinamide 2,2,2-trifluoroacetate (2 mg, 0.23% yield). LCMS (ESI) m/z 499.8 (M+H)⁺; ¹H NMR (500 MHz, METHANOL-d₄) δ ppm 1.52 (s, 4H), 1.95 (dd, J=4.58, 1.53 Hz, 3H), 2.18 (dd, J=4.58, 1.53 Hz, 2H), 4.07 (s, 2H), 6.71-7.07 (m, 1H), 7.97 (d, J=7.33 Hz, 1H), 8.13 (s, 1H), 8.26 (t, J=7.63 Hz, 1H), 8.40 (d, J=7.94 Hz, 1H), 8.91 (s, 1H), 9.52 (d, J=1.22 Hz, 1H)

### Example 275: N-(2-(tetrahydro-2H-pyran-4-yl)imidazo[1,2-a]pyrazin-6-yl)-2,3-dihydrobenzofuran-7-carboxamide

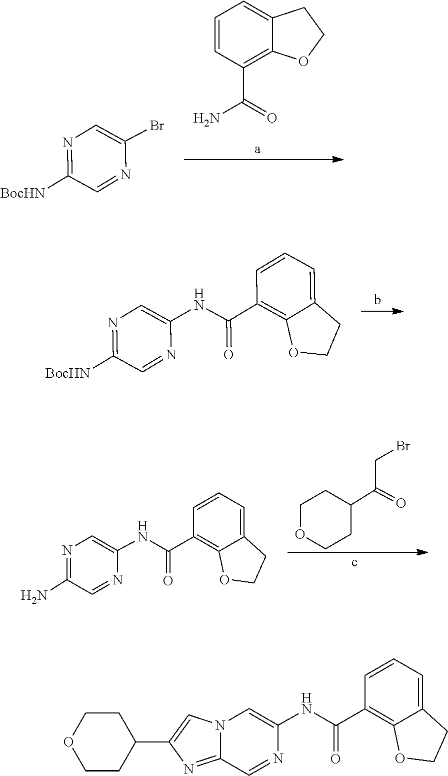

Step a: To a solution of tert-butyl (5-bromopyrazin-2-yl)carbamate (200 mg, 729.63 μmol) in toluene (5.00 mL) was added 2,3-dihydrobenzofuran-7-carboxamide (130.96 mg, 802.59 μmol), Pd₂(dba)₃ (66.81 mg, 72.96 μmol), XantPhos (84.44 mg, 145.93 μmol) and Cs₂CO₃ (475.46 mg, 1.46 mmol) and the reaction was stirred at 105° C. under N₂ for 12 h. The cooled mixture was concentrated in vacuo and the residue was purified by silica gel chromatography using Combiflash® (PE/EtOAc=10/1 to 3/1) to give tert-butyl (5-(2,3-dihydrobenzofuran-7-carboxamido)pyrazin-2-yl)carbamate (140 mg, 51.15% yield) as yellow solid. LCMS (ESI) m/z 357.2 (M+H)⁺;

Step b: To a solution of tert-butyl (5-(2,3-dihydrobenzofuran-7-carboxamido)pyrazin-2-yl)carbamate (140 mg, 392.84 μmol) in DCM (5 mL) was added TFA (5 mL) and the reaction stirred at 10° C. for 2 h. The reaction was neutralized using aq. NaHCO₃ then extracted with DCM (20 mL×3). The combined organic extracts were dried over Na₂SO₄, filtered and evaporated under reduced pressure to give N-(5-aminopyrazin-2-yl)-2,3-dihydrobenzofuran-7-carboxamide (100 mg, crude) as a yellow solid. LCMS (ESI) m/z 257.1 (M+H)⁺;

Step c: To a solution of N-(5-aminopyrazin-2-yl)-2,3-dihydrobenzofuran-7-carboxamide (50 mg, 195.11 μmol) in DMSO (500 μL) and MeCN (2 mL) was added 2-bromo-1-(tetrahydro-2H-pyran-4-yl)ethan-1-one (121.20 mg, 585.33 μmol) and NaHCO₃ (32.78 mg, 390.22 μmol) and the reaction irradiated in the microwave at 115° C. for 2 h. The cooled mixture was concentrated in vacuo and the residue was purified by prep-HPLC (Column: Phenomenex Synergi C18 100×30 mm×3 um) eluting with water/(10 mM NH₄HCO₃-MeCN from 29% to 59%) to give N-(2-(tetrahydro-2H-pyran-4-yl)imidazo[1,2-a]pyrazin-6-yl)-2,3-dihydrobenzofuran-7-carboxamide (6.90 mg, 9.42% yield) as yellow solid. LCMS (ESI) m/z 365.0 (M+H)⁺; ¹H NMR (500 MHz, METHANOL-d₄) δ ppm: 1.90 (qd, J=12.13 Hz, 3.74 Hz, 2H), 2.05 (d, J=11.75 Hz, 2H), 3.12 (t, J=11.67 Hz, 1H), 3.36-3.40 (m, 4H), 3.64 (t, J=11.75 Hz, 2H), 4.08 (d, J=7.63 Hz, 2H), 7.06 (t, J=7.55 Hz, 1H), 7.50 (d, J=7.17 Hz, 1H), 7.89 (d, J=7.48 Hz, 1H), 7.98 (s, 1H), 8.78 (s, 1H), 9.43 (s, 1H).

### Example 276 and 277: 6-(Difluoromethyl)-N-(8-methoxy-2-((1S*,4R*)-1-methyl-2-oxabicyclo[2.2.1]heptan-4-yl)imidazo[1,2-a]pyrazin-6-yl)picolinamide and 6-(Difluoromethyl)-N-(8-methoxy-2-((1R*,4S*)-1-methyl-2-oxabicyclo[2.2.1]heptan-4-yl)imidazo[1,2-a]pyrazin-6-yl)picolinamide

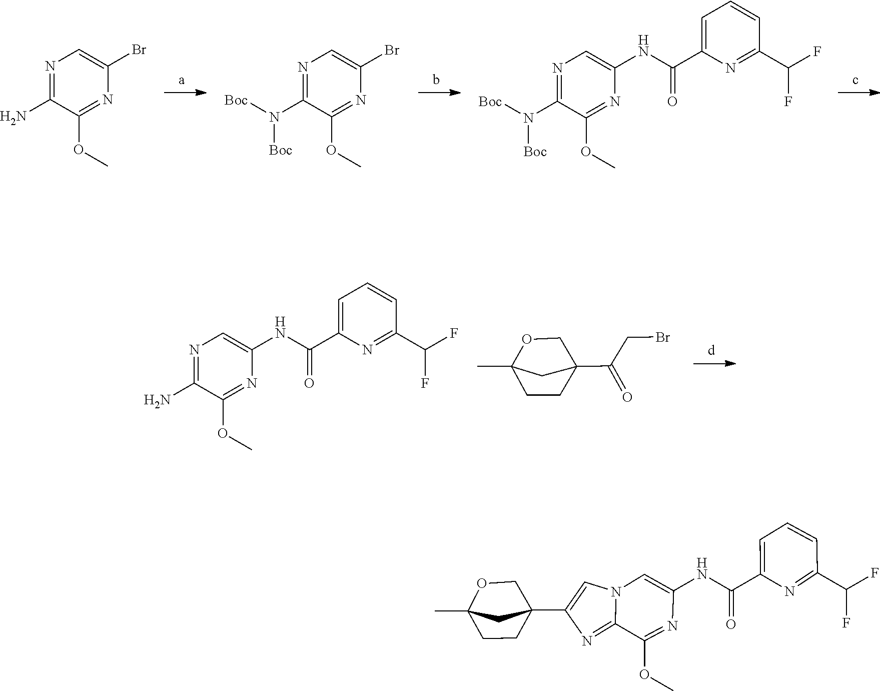

Step a: To a solution of 5-bromo-3-methoxypyrazin-2-amine (4.0 g, 19.6 mmol) in DCM (39 mL) were added DMAP (2.4 g, 19.6 mmol), TEA (8.1 mL, 58.8 mmol) and (Boc)₂O (18 mL, 78.4 mmol) and the reaction stirred at 25° C. overnight. The reaction was concentrated in vacuo and the crude purified by silica gel column chromatography (dry load, 3:1 EtOAC/EtOH in heptanes, 0-100%) to give tert-butyl (5-bromo-3-methoxypyrazin-2-yl)(tert-butoxycarbonyl)carbamate (7.5 g, 94% yield). LCMS (ESI) m/z 250.0 (M-Boc-tBu+H)⁺.

Step b: A vial charged with tert-butyl (5-bromo-3-methoxypyrazin-2-yl)(tert-butoxycarbonyl)carbamate (1.5 g, 3.71 mmol), 6-(difluoromethyl)picolinamide (958 mg, 5.57 mmol), Pd₂(dba)₃ (340 mg, 0.37 mmol), XantPhos (429 mg, 0.74 mmol) and Cs₂CO₃ (2.42 g, 7.42 mmol) was purged with N₂ and closed with a screw cap with septa. Toluene (10.6 mL) was added via syringe and the reaction heated at 100° C. for 16 h. The cooled mixture was quenched with water, the layers separated and the aqueous phase extracted with EtOAc (3×). The combined organic extracts were dried over MgSO₄, filtered and concentrated in vacuo. The crude material was purified by silica gel column chromatography (dry load, 3:1 EtOAc/EtOH in heptanes, 0-40%) to give tert-butyl (tert-butoxycarbonyl)(5-(6-(difluoromethyl)picolinamido)-3-methoxypyrazin-2-yl)carbamate (1.67 g, 91% yield). LCMS (ESI) m/z 340.1 (M+H)⁺.

Step c: A mixture of TFA (4.6 mL, 60.5 mmol) and tert-butyl (tert-butoxycarbonyl)(5-(6-(difluoromethyl)picolinamido)-3-methoxypyrazin-2-yl)carbamate (3.0 g, 6.05 mmol) in DCM (12.1 mL) was stirred for 16 h at rt. The reaction was concentrated in vacuo, the residue diluted with EtOAc and aq. sat. solution of NaHCO₃ and the layers separated. The aqueous phase was extracted with EtOAc (3×), the combined organic extracts washed with bine, dried over MgSO₄, filtered and evaporated under reduced pressure to give N-(5-amino-6-methoxy-pyrazin-2-yl)-6-(difluoromethyl)picolinamide (840 mg, 47% yield), which was used without further purification. LCMS (ESI) m/z 296.1 (M+H)⁺.

Step d: A mixture of N-(5-amino-6-methoxy-pyrazin-2-yl)-6-(difluoromethyl)picolinamide (150 mg, 508 μmol), 2-bromo-1-(1-methyl-2-oxabicyclo[2.2.1]heptan-4-yl)ethanone (177 mg, 762 μmol) and NaHCO₃ (128 mg, 1.52 mmol) in MeCN (2.0 mL) and toluene (1.3 mL) was heated at 90° C. for 16 h. The cooled reaction was filtered through Celite®, the filtrate concentrated in vacuo and the crude material purified by TFA modified reverse-phase HPLC eluting to provide 6-(difluoromethyl)-N-(8-methoxy-2-(1-methyl-2-oxabicyclo[2.2.1]heptan-4-yl)imidazo[1,2-a]pyrazin-6-yl)picolinamide 2,2,2-trifluoroacetate (100 mg, 46% yield). This product was further purified by SFC using a CHIRALPAK AD-H 30×250 mm, 5 μm column; eluting with 45% EtOH w/0.1% DEA in CO₂; flow rate: 100 mL/min) to give 6-(difluoromethyl)-N-(8-methoxy-2-((1S*,4R*)-1-methyl-2-oxabicyclo[2.2.1]heptan-4-yl)imidazo[1,2-a]pyrazin-6-yl)picolinamide (16.6 mg, 99% purity, 100% ee). The stereochemistry of this enantiomer was arbitrarily assigned. LCMS (ESI) m/z 430.3 (M+H)⁺. ¹H NMR (400 MHz, CDCl₃) δ ppm 1.47 (s, 3H), 1.79-1.89 (m, 2H), 1.93-1.98 (m, 1H), 2.01-2.05 (m, 1H), 2.09-2.22 (m, 2H), 4.00 (d, J=6.53 Hz, 1H), 4.12 (dd, J=6.53, 3.26 Hz, 1H), 4.20 (s, 3H), 6.61-6.94 (m, 1H), 7.46 (s, 1H), 7.88 (d, J=7.28 Hz, 1H), 8.12 (t, J=7.78 Hz, 1H), 8.41 (dd, J=7.78, 0.75 Hz, 1H), 8.95 (s, 1H), 9.86 (s, 1H).

Further elution provided diastereoisomer 2.

¹H NMR (400 MHz, CDCl₃ δ ppm 1.47 (s, 3H), 1.78-1.87 (m, 2H), 1.93-1.99 (m, 1H), 2.00-2.06 (m, 1H), 2.11-2.21 (m, 2H), 4.00 (d, J=6.53 Hz, 1H), 4.12 (dd, J=6.53, 3.26 Hz, 1H), 4.19-4.24 (m, 3H), 6.60-6.93 (m, 1H), 7.46 (s, 1H), 7.88 (d, J=7.78 Hz, 1H), 8.12 (t, J=7.78 Hz, 1H), 8.41 (dd, J=7.78, 0.75 Hz, 1H), 8.91-9.02 (m, 1H), 9.86 (s, 1H)

Example 278: N-(2-(1,4-dioxan-2-yl)-8-methoxyimidazo[1,2-a]pyrazin-6-yl)-6-(difluoromethyl)picolinamide was prepared under standard condensation reaction conditions with A mixture of Example 277, step c: N-(5-amino-6-methoxy-pyrazin-2-yl)-6-(difluoromethyl)picolinamide (50 mg, 169.35 μmol), and 2-chloro-1-(1,4-dioxan-2-yl)ethan-1-one (64.55 mg, 372.57 μmol) to give N-(2-(1,4-dioxan-2-yl)-8-methoxyimidazo[1,2-a]pyrazin-6-yl)-6-(difluoromethyl)picolinamide (9.30 mg, 12.87% yield). LCMS (ESI) m/z 406.2 (M+H)⁺; ¹H NMR (500 MHz, DMSO-d₆) δ ppm 3.54-3.63 (m, 2H), 3.75-3.83 (m, 2H), 3.86-3.90 (m, 1H), 3.99 (dd, J=11.60, 3.05 Hz, 1H), 4.11 (s, 3H), 4.74 (dd, J=10.07, 2.75 Hz, 1H), 7.07-7.31 (m, 1H), 8.04 (dd, J=7.32, 1.83 Hz, 1H), 8.19 (s, 1H), 8.29-8.37 (m, 2H), 9.08 (s, 1H), 9.98 (s, 1H).

### Example 279: 6-(difluoromethyl)-N-(7-isopropoxy-2-(1-methyl-2-oxabicyclo[2.1.1]hexan-4-yl)imidazo[1,2-a]pyrimidin-6-yl)picolinamid

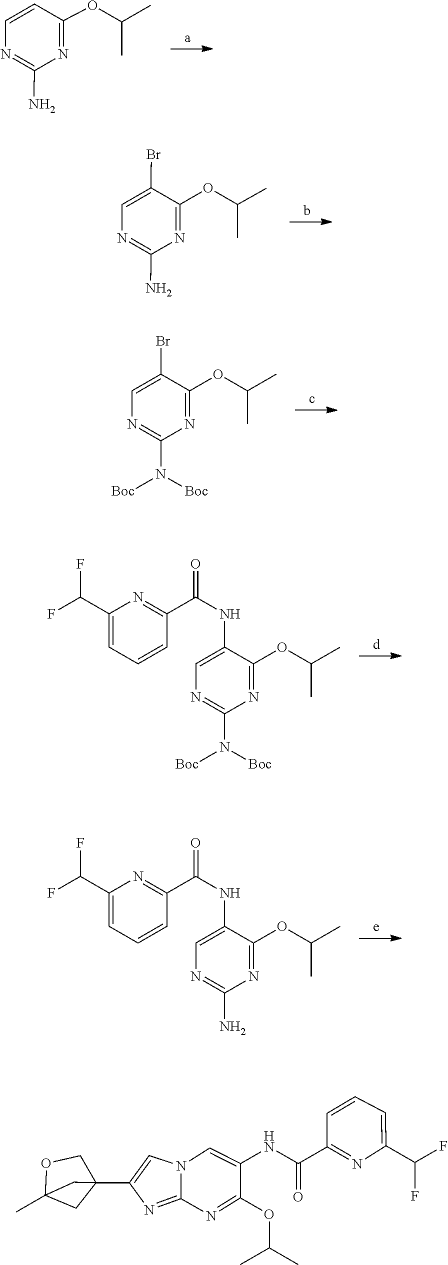

Step a: 4-Isopropoxypyrimidin-2-amine (5.90 g, 38.52 mmol) in CHCl₃ (256.8 mL) was treated with NBS (6.86 g, 38.52 mmol) and the reaction stirred overnight in the absence of light. The reaction was washed with sat. aq. NaHCO₃, the layers separated and the organic layer was dried and evaporated under reduced pressure to give 5-bromo-4-isopropoxypyrimidine-2-amine, which was used without further purification. LCMS (ESI) m/z 232.0 (M+H)⁺.

Step b: To a solution of 5-bromo-4-isopropoxypyrimidin-2-amine (8.82 g, 38.0 mmol) and (Boc)₂O (24.88 g, 114.0 mmol) in DCM (95.0 mL) were added DMAP (4.64 g, 38.0 mmol) and TEA (15.81 mL, 114.0 mmol) and the reaction stirred at 25° C. overnight. The reaction mixture was concentrated in vacuo and the crude purified by column chromatography to give tert-butyl (5-bromo-4-isopropoxypyrimidin-2-yl)(tert-butoxycarbonyl)carbamate (5.80 g, 35.31% yield). ¹H NMR (500 MHz, CDCl₃) δ ppm 1.42 (d, J=6.10 Hz, 8H) 1.47-1.53 (m, 32H) 5.35 (d, J=6.10 Hz, 1H) 8.55 (s, 1H) Step c: A mixture of tert-butyl (5-bromo-4-isopropoxypyrimidin-2-yl)(tert-butoxycarbonyl)carbamate (2.00 g, 4.63 mmol), XantPhos (1.07 g, 1.85 mmol), Cs₂CO₃ (3.02 g, 9.26 mmol), Pd₂(dba)₃ (847.96 mg, 926.00 μmol) and 6-(difluoromethyl)pyridine-2-carboxamide (1.59 g, 9.26 mmol) was purged under N₂ and the reaction vial sealed with a septa. Dioxane (46.30 mL) was added, the vial was sealed and the reaction heated at 80° C. overnight. The reaction was cooled to rt, silica gel added, the mixture concentrated in vacuo, and the powder purified by column chromatography (5-100% EtOAc in Heptanes) to obtain tert-butyl (tert-butoxycarbonyl)(5-(6-(difluoromethyl)picolinamido)-4-isopropoxypyrimidin-2-yl)carbamate (1.30 g, 53.63% yield). LCMS (ESI) m/z 523.9 (M+H)⁺.

Step d: tert-Butyl (tert-butoxycarbonyl)(5-(6-(difluoromethyl)picolinamido)-4-isopropoxypyrimidin-2-yl)carbamate (1.30 g, 2.48 mmol) was dissolved in DCM (8.27 mL), TFA (5.66 g, 49.60 mmol) added and the solution stirred at rt for 1 hr. The solution was concentrated in vacuo to give N-(2-amino-4-isopropoxypyrimidin-5-yl)-6-(difluoromethyl)picolinamide 2,2,2-trifluoroacetate. LCMS (ESI) m/z 323.9 (M+H)⁺.

Step e: A mixture of N-(2-amino-4-isopropoxypyrimidin-5-yl)-6-(difluoromethyl)picolinamide trifluoroacetate (295.14 mg, 0.913 mmol), 2-bromo-1-(1-methyl-2-oxabicyclo[2.1.1]hexan-4-yl)ethanone (200.0 mg, 0.913 mmol) and NaHCO₃ (230.08 mg, 2.74 mmol) in EtOH (2.28 mL) was heated at 80° C. overnight. The reaction was cooled to rt, filtered, concentrated in vacuo, and the residue purified by TFA modified mass directed prep HPLC to give 6-(difluoromethyl)-N-(7-isopropoxy-2-(1-methyl-2-oxabicyclo[2.1.1]hexan-4-yl)imidazo[1,2-a]pyrimidin-6-yl)picolinamide 2,2,2-trifluoroacetate (78.10 mg, 15.37% yield). LCMS (ESI) m/z 443.9 (M+H)⁺. ¹H NMR (500 MHz, METHANOL-d₄) δ ppm 1.52 (s, 3H) 1.60 (d, J=6.10 Hz, 6H) 1.97 (dd, J=4.58, 1.53 Hz, 2H) 2.18 (d, J=4.88 Hz, 2H) 4.03 (s, 2H) 5.63 (quin, J=6.10 Hz, 1H) 6.71-7.03 (m, 1H) 7.86 (s, 1H) 7.99 (d, J=7.94 Hz, 1H) 8.28 (t, J=7.94 Hz, 1H) 8.40 (d, J=7.94 Hz, 1H)

### Example 280: 3-(difluoromethyl)-N-(2-morpholinoimidazo[1,2-a]pyridin-6-yl)benzamide

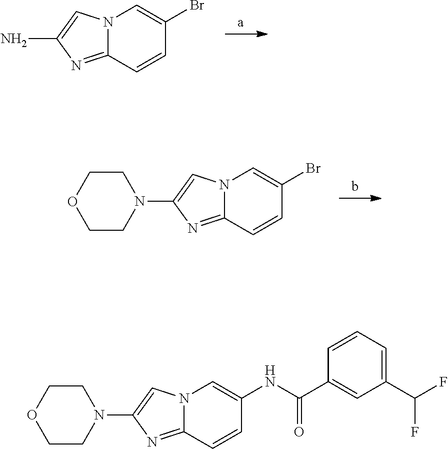 [SMILES: Nc1cn2cc(Br)ccc2n1 ; Brc1ccc2nc(N3CCOCC3)cn2c1 ; O=C(Nc1ccc2nc(N3CCOCC3)cn2c1)c1cccc(C(F)F)c1]

Step a: 1-Bromo-2-(2-bromoethoxy)ethane (1.07 g, 4.62 mmol) and DIPEA (1.19 g, 9.24 mmol) were added to a solution of 6-bromoimidazo[1,2-a]pyridin-2-amine trifluoroacetate (1.0 g, 3.08 mmol) in DMA (10.27 mL) and the reaction heated at 120° C. overnight. The cooled solution was diluted with brine, extracted with EtOAc, dried, concentrated in vacuo, and purified by column chromatography to give 4-(6-bromoimidazo[1,2-a]pyridin-2-yl)morpholine (191 mg, 21.98% yield). ¹H NMR (400 MHz, CDCl₃) δ ppm 3.21-3.34 (m, 4H), 3.77-3.92 (m, 5H), 6.85 (s, 1H), 7.06-7.20 (m, 1H), 7.28 (d, J=0.75 Hz, 2H), 8.07-8.23 (m, 1H).

Step b: A mixture of 4-(6-bromoimidazo[1,2-a]pyridin-2-yl)morpholine (191.01 mg, 677 μmol), Pd(OAc)₂ (30.40 mg, 135.40 μmol), XantPhos (156.69 mg, 270.80 μmol), Cs₂CO₃ (441.16 mg, 1.35 mmol) and 6-(difluoromethyl)picolinamide (291.33 mg, 1.69 mmol) was purged with N₂, dioxane (6.77 mL) added, the reaction vessel sealed and heated at 100° C. overnight. The cooled reaction mixture was filtered through Celite® and the filtrate concentrated in vacuo. The crude product was purified by prep. HPLC (XSelect CSH Prep C18 OBD 5 μm 50×100 mm column) eluting with H₂O/0.2% NH₄OH in MeCN (10-55%) over 15 min at 80 mL/min), to obtain 3-(difluoromethyl)-N-(2-morpholinoimidazo[1,2-a]pyridin-6-yl)benzamide (5.0 mg, 1.98% yield). LCMS (ESI) m/z 374.2 (M+H)⁺; ¹H NMR (500 MHz, METHANOL-d₄) δ ppm 3.21-3.28 (m, 4H), 3.81-3.94 (m, 4H), 6.78-7.09 (m, 1H), 7.25 (s, 1H), 7.35 (d, J=9.16 Hz, 1H), 7.40-7.50 (m, 1H), 7.95 (d, J=7.94 Hz, 1H), 8.24 (s, 1H), 8.32-8.42 (m, 1H), 9.18 (d, J=1.83 Hz, 1H).

### Example 281: 1-(difluoromethyl)-N-(8-methoxy-2-(tetrahydro-2H-pyran-4-yl)imidazo[1,2-a]pyridin-6-yl)-1H-pyrazole-3-carboxamide

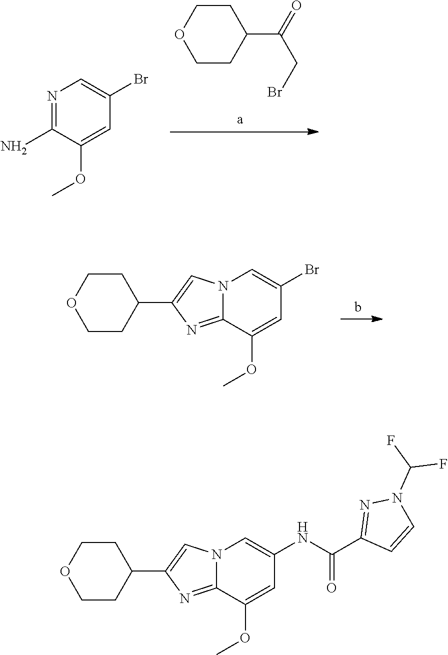

Step a: A mixture of 5-bromo-3-methoxypyridin-2-amine (1.00 g, 4.93 mmol), 2-bromo-1-(tetrahydro-2H-pyran-4-yl)ethan-1-one (1.02 g, 4.93 mmol) and NaHCO₃ (1.24 g, 14.79 mmol) in EtOH (12.32 mL) was heated at 80° C. overnight. The mixture was cooled to rt, silica gel added, the mixture concentrated in vacuo and the powder purified by silica gel chromatography via dry load eluting with 0-50% 3:1 EtOAC/EtOH in Hept to give 6-bromo-8-methoxy-2-(tetrahydro-2H-pyran-4-yl)imidazo[1,2-a]pyridine (740 mg, 48.24% yield). LCMS (ESI) m/z 313 (M+H)⁺;

Step b: A mixture of 6-bromo-8-methoxy-2-(tetrahydro-2H-pyran-4-yl)imidazo[1,2-a]pyridine (200 mg, 642.74 μmol), XantPhos (148.76 mg, 257.10 μmol), Pd(OAc)₂ (28.86 mg, 128.55 μmol), Cs₂CO₃ (418.84 mg, 1.29 mmol) and 1-(difluoromethyl)-1H-pyrazole-3-carboxamide (310.65 mg, 1.93 mmol) was purged with N₂, dioxane (4.28 mL) added, the reaction vessel sealed was sealed and heated at 100° C. overnight. The mixture was cooled to rt, filtered through Celite®, the filtrate concentrated in vacuo and the residue purified by TFA-modified mass directed HPLC to give 1-(difluoromethyl)-N-(8-methoxy-2-(tetrahydro-2H-pyran-4-yl)imidazo[1,2-a]pyridin-6-yl)-1H-pyrazole-3-carboxamide (8.90 mg, 22.74 μmol, 3.54% yield). LCMS (ESI) m/z 392.2 (M+H)⁺; ¹H NMR (400 MHz, CDCl₃) δ ppm 1.83-1.92 (m, 2H), 2.09 (br dd, J=12.93, 1.88 Hz, 2H), 3.04-3.12 (m, 1H), 3.54-3.61 (m, 2H), 4.03 (s, 3H), 4.07-4.11 (m, 1H), 4.07-4.11 (m, 1H), 6.43 (s, 1H), 7.10 (d, J=2.76 Hz, 1H), 7.24 (s, 1H), 7.34-7.40 (m, 1H), 7.93 (d, J=2.76 Hz, 1H), 8.53 (br s, 1H), 8.79 (d, J=1.51 Hz, 1H).

### Example 282: 6-(difluoromethyl)-N-(8-methoxy-2-(3-methoxypropyl)imidazo[1,2-a]pyridin-6-yl)picolinamide

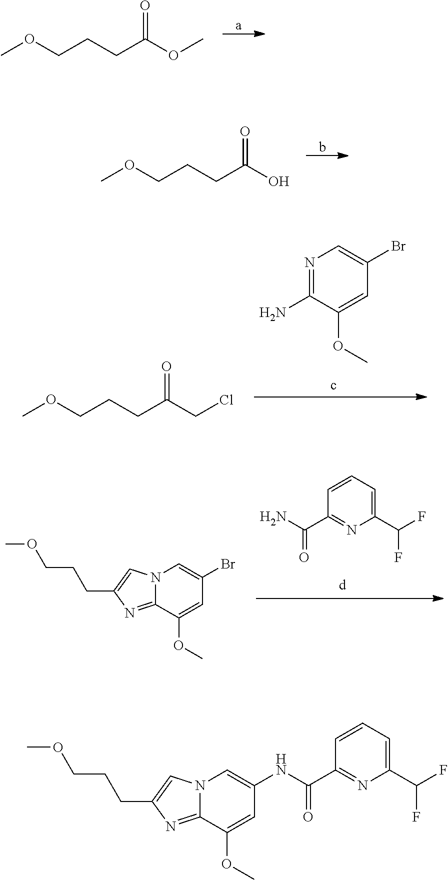

Step a: To a solution of methyl 4-methoxybutanoate (5 g, 37.83 mmol) in THF (50 mL) was added LiOH.H₂O (3.17 g, 75.66 mmol) and water (50 mL) and the reaction was stirred at 20° C. for 12 h. The mixture was acidified with aqueous HCl to pH<2, then extracted with DCM (50 mL×2). The combined organic extracts were washed with water (30 mL), dried over Na₂SO₄, filtered and evaporated under reduced pressure to give 4-methoxybutanoic acid (4 g, 89% yield) as a white solid. ¹H NMR (500 MHz, CDCl₃) δ ppm: 1.89-1.94 (m, 2H), 2.47 (t, J=7.0 Hz, 2H), 3.35 (s, 3H), 3.44 (t, J=6.0 Hz, 2H), 11.37 (br s, 1H).

Step b: To a solution of 4-methoxybutanoic acid (2 g, 16.93 mmol) in DCM (20 mL) was added (COCl)₂ (4.3 g, 33.86 mmol) and the reaction stirred at 20° C. for 4 h. The mixture was filtered, the filtrate concentrated in vacuo and the residue was dissolved in THF (20 mL) and MeCN (20 mL). TMSCHN₂ (12.63 mL, 25.26 mmol, 2 M in hexane) was added, the mixture was stirred at 0° C. for 1 h then 12 M HCl (4.2 mL, 50.43 mmol) was added. The reaction was stirred at 0° C. for 30 mins, basified with aq. NaHCO₃ to pH>7 and the mixture extracted with DCM (20 mL×2).

The organic layer was washed with water (20 mL), dried over Na₂SO₄, filtered and evaporated under vacuum to give 1-chloro-5-methoxypentan-2-one (2.2 g, 86% yield) as a colorless liquid. ¹H NMR (500 MHz, CDCl₃) δ ppm: 1.94-1.88 (m, 2H), 2.49 (t, J=7.0 Hz, 2H), 3.31 (s, 3H), 3.40 (t, J=6.0 Hz, 2H), 4.11 (s, 2H).

Step c: To a solution of 1-chloro-5-methoxypentan-2-one (500 mg, 2.46 mmol) and 5-bromo-3-methoxypyridin-2-amine (445.04 mg, 2.96 mmol) in EtOH (10 mL) was added KI (81.76 mg, 0.49 mmol) and NaHCO₃ (620.64 mg, 7.39 mmol) and the reaction stirred at 80° C. for 4 h. The cooled mixture was evaporated under reduced pressure and the residue was purified by silica gel chromatography (PE/EtOAc from 2/1 to 0/1) to give 6-bromo-8-methoxy-2-(3-methoxypropyl)imidazo[1,2-a]pyridine (300 mg, 40% yield) as a brown liquid. ¹H NMR (500 MHz, CDCl₃) δ: 2.00-2.07 (m, 2H), 2.84 (t, J=7.0 Hz, 2H), 3.34 (s, 3H), 3.53 (t, J=6.5 Hz, 2H), 3.99 (s, 3H), 6.50-6.51 (m, 1H), 7.29 (s, 1H), 7.83-7.85 (m, 1H).

Step d: To a mixture of 6-bromo-8-methoxy-2-(3-methoxypropyl)imidazo[1,2-a]pyridine (100 mg, 0.33 mmol) and 6-(difluoromethyl)picolinamide (57.54 mg, 0.33 mmol) in toluene (5 mL) was added Pd(OAc)₂ (7.5 mg, 0.03 mmol), XantPhos (38.68 mg, 0.07 mmol) and Cs₂CO₃ (217.82 mg, 0.57 mmol). The mixture was stirred at 110° C. for 4 h and the cooled mixture then evaporated under reduced pressure. The residue was purified by prep-HPLC (Phenomenex Synergi C18 150×30 mm×4 μm, water (0.05% HCl)-MeOH from 25% to 45%) to give 6-(difluoromethyl)-N-(8-methoxy-2-(3-methoxypropyl)imidazo[1,2-a]pyridin-6-yl)picolinamide hydrochloride (12 mg, 9% yield) as a white solid. LCMS (ESI) m/z 391.0 (M+H)⁺; ¹H NMR (400 MHz, METHANOL) δ: 2.00-2.08 (m, 2H), 2.98-2.93 (m, 2H), 3.36 (s, 3H), 3.50 (t, J=6.0 Hz, 2H), 4.19 (s, 3H), 6.93 (t, J=54.8 Hz, 1H), 7.79 (s, 1H), 7.97 (d, J=7.6 Hz, 1H), 8.05 (s, 1H), 8.25 (t, J=8.0 Hz, 1H), 8.37-8.40 (m, 1H), 9.33-9.34 (m, 1H).

### Example 283 and 284: N-(2-((1S,2S)-2-cyanocyclobutyl)-8-methoxyimidazo[1,2-a]pyridin-6-yl)-6-(difluoromethyl)picolinamide and N-(2-((1R,2R)-2-cyanocyclobutyl)-8-methoxyimidazo[1,2-a]pyridin-6-yl)-6-(difluoromethyl)picolinamide

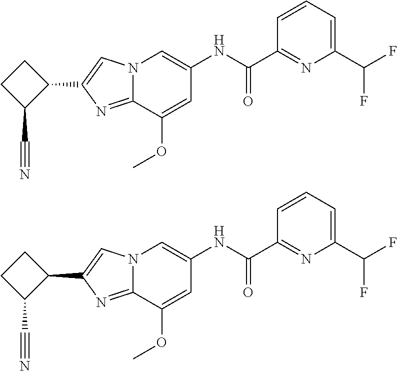 [SMILES: COc1cc(NC(=O)c2cccc(C(F)F)n2)cn2cc([C@H]3CC[C@@H]3C#N)nc12 ; COc1cc(NC(=O)c2cccc(C(F)F)n2)cn2cc([C@@H]3CC[C@H]3C#N)nc12]

N-(2-(2-cyanocyclobutyl)-8-methoxyimidazo[1,2-a]pyridin-6-yl)-6-(difluoromethyl)picolinamide was prepared from trans 2-cyanocyclobutane-1-carboxylic acid, 5-bromo-3-methoxypyridin-2-amine and 6-(difluoromethyl)picolinamide, following a similar synthetic sequence to that described in Example 281. It was purified further by SFC using a CHIRALPAK AD-H 30×250 mm, 5 μm column, eluting with 45% MeOH w/0.1% DEA in CO₂ (flow rate: 100 mL/min, ABPR 120 bar, MBPR 40 psi, column temp 40° C.) to provide diastereoisomer 1, 18.7 mg. ¹H NMR (400 MHz, CDCl₃) δ ppm 2.31-2.40 (m, 3H) 2.59-2.67 (m, 1H) 3.58-3.65 (m, 1H) 3.89-3.95 (m, 1H) 4.08 (s, 3H) 6.51 (d, J=1.51 Hz, 1H) 6.60-6.90 (m, 1H) 7.39-7.44 (m, 1H) 7.86 (d, J=7.78 Hz, 1H) 8.11 (t, J=7.78 Hz, 1H) 8.40 (d, J=7.28 Hz, 1H) 8.87 (d, J=1.51 Hz, 1H) 9.65 (s, 1H)

Further elution provided diastereoisomer 2, 17.2 mg. ¹H NMR (400 MHz, CDCl₃ δ ppm 2.30-2.41 (m, 3H) 2.57-2.66 (m, 1H) 3.57-3.65 (m, 1H) 3.87-3.95 (m, 1H) 4.08 (s, 3H) 6.51 (d, J=1.51 Hz, 1H) 6.60-6.89 (m, 1H) 7.43 (s, 1H) 7.86 (d, J=7.78 Hz, 1H) 8.11 (t, J=7.78 Hz, 1H) 8.40 (dd, J=8.03, 0.75 Hz, 1H) 8.87 (d, J=1.51 Hz, 1H) 9.65 (s, 1H).

The stereochemistry was arbitrarily assigned.

### Example 285 and 286: 6-(difluoromethyl)-N-(8-ethoxy-2-(tetrahydro-2H-pyran-3-yl)imidazo[1,2-a]pyridin-6-yl)picolinamide, Enantiomer 1 and 2

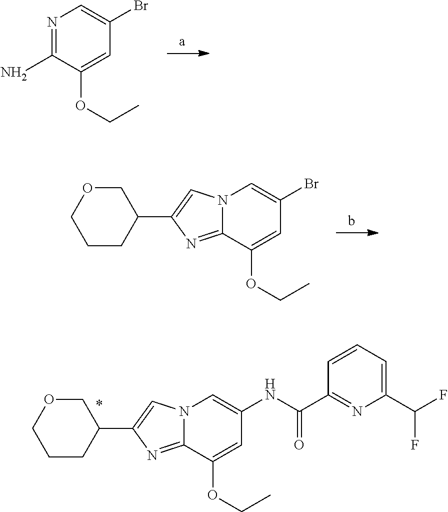 [SMILES: CCOc1cc(Br)cnc1N ; CCOc1cc(Br)cn2cc(C3CCCOC3)nc12 ; CCOc1cc(NC(=O)c2cccc(C(F)F)n2)cn2cc(C3CCCOC3)nc12]

Step a: A mixture of 5-bromo-3-ethoxypyridin-2-amine (1.00 g, 4.61 mmol), 2-bromo-1-(tetrahydro-2H-pyran-3-yl)ethan-1-one (954.55 mg, 4.61 mmol) and NaHCO₃ (1.16 g, 13.83 mmol) in MeCN (9.22 mL) was heated at 80° C. overnight. The reaction was cooled to rt, filtered through a frit and the filtrate concentrated in vacuo. The residue was purified by column chromatography on silica gel eluting with (0-30% EtOAc in Hept) to obtain 6-bromo-8-ethoxy-2-(tetrahydro-2H-pyran-3-yl)imidazo[1,2-a]pyridine (715 mg, 2.20 mmol, 47.69% yield). LCMS (ESI) m/z 324.9 (M+H)⁺;

Step b: A vial charged with 6-bromo-8-ethoxy-2-(tetrahydro-2H-pyran-3-yl)imidazo[1,2-a]pyridine (260 mg, 799.51 μmol), XantPhos (185.04 mg, 319.80 μmol), Cs₂CO₃ (520.99 mg, 1.60 mmol), Pd(OAc)₂ (35.90 mg, 159.90 μmol) and 6-(difluoromethyl)picolinamide (412.86 mg, 2.40 mmol) was purged with N₂ and closed with a screw cap with septa. Dioxane (5.33 mL) was added, the vial sealed with and heated at 100° C. overnight. The cooled reaction mixture was filtered and concentrated in vacuo. The crude product was purified by HPLC using a Sunfire Prep C18 OBD 5 μm 50×100 mm column eluting with H₂O (0.1% TFA in MeCN from 5 to 55%) over 12 min (flow rate: 50 mL/min) to give 6-(difluoromethyl)-N-(8-ethoxy-2-(tetrahydro-2H-pyran-3-yl)imidazo[1,2-a]pyridin-6-yl)picolinamide, 35.3 mg, 10.66%.

This product was further purified by SFC using a CHIRALPAK IB 30×250 mm, 5 μm column, eluting with 40% MeOH w/0.1% DEA in CO2 (flow rate: 100 mL/min, ABPR 120 bar, MBPR 40 psi, column temp 40° C.) to provide enantiomer 1: ¹H NMR (400 MHz, CDCl₃) δ ppm 1.60 (t, J=7.03 Hz, 3H), 1.71-1.90 (m, 4H) 2.18-2.27 (m, 1H), 3.11-3.24 (m, 1H), 3.53-3.64 (m, 2H), 3.88-3.96 (m, 1H), 4.20 (dd, J=11.04, 3.01 Hz, 1H), 4.30 (q, J=7.03 Hz, 2H), 6.48 (s, 1H), 6.59-6.89 (m, 1H), 7.40 (s, 1H), 7.86 (d, J=7.78 Hz, 1H), 8.12 (t, J=7.78 Hz, 1H), 8.41 (d, J=7.78 Hz, 1H), 8.87 (s, 1H), 9.63 (s, 1H).

Further elution gave enantiomer 2: ¹H NMR (400 MHz, CDCl₃) δ ppm 1.60 (t, J=7.03 Hz, 3H), 1.70-1.87 (m, 4H), 2.14-2.26 (m, 1H), 3.10-3.23 (m, 1H), 3.51-3.64 (m, 2H), 3.89-3.98 (m, 1H), 4.20 (dd, J=10.92, 2.64 Hz, 1H), 4.30 (q, J=7.03 Hz, 2H), 6.47 (s, 1H), 6.57-6.89 (m, 1H), 7.40 (s, 1H), 7.86 (d, J=7.78 Hz, 1H), 8.11 (t, J=7.78 Hz, 1H), 8.41 (d, J=7.78 Hz, 1H), 8.87 (s, 1H), 9.62 (s, 1H)

### Example 287 and 288: N-(8-ethoxy-2-(tetrahydro-2H-pyran-3-yl)imidazo[1,2-a]pyridin-6-yl)-6-(trifluoromethyl)picolinamide, Enantiomer 1 and 2

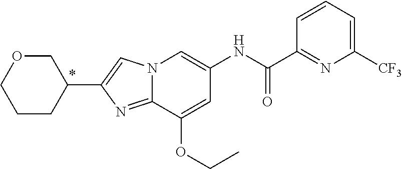 [SMILES: CCOc1cc(NC(=O)c2cccc(C(F)(F)F)n2)cn2cc(C3CCCOC3)nc12]

N-(8-ethoxy-2-(tetrahydro-2H-pyran-3-yl)imidazo[1,2-a]pyridin-6-yl)-6-(trifluoromethyl)picolinamide was prepared from 5-bromo-3-ethoxypyridin-2-amine and (6-trifluoromethyl)picolinamide following the method described in Example 285 and 286. LCMS (ESI) m/z 435.2 (M+H)⁺;

The compound was further purified by SFC using a CHIRALPAK OX—H 30×250 mm, 5 μm column, eluting with 40% EtOH w/0.1% DEA in CO₂ (flow rate: 100 mL/min, ABPR 120 bar, MBPR 40 psi, column temp 40° C.) to give enantiomer 1. ¹H NMR (400 MHz, CDCl₃) δ ppm 1.59-1.70 (m, 3H), 1.71-1.91 (m, 3H), 2.26 (br s, 1H), 3.10-3.26 (m, 1H), 3.54-3.65 (m, 2H), 3.93-4.02 (m, 1H), 4.24 (br d, J=10.29 Hz, 1H), 4.30-4.39 (m, 2H), 6.48 (br s, 1H), 7.41 (s, 1H), 7.89-7.99 (m, 1H), 8.15-8.23 (m, 1H), 8.51 (br d, J=7.78 Hz, 1H), 8.87 (br s, 1H), 9.59 (br s, 1H).

Further elution gave enantiomer 2. ¹H NMR (400 MHz, CDCl₃) δ ppm 1.62 (t, J=7.03 Hz, 3H), 1.71-1.88 (m, 3H), 2.18-2.27 (m, 1H), 3.13-3.24 (m, 1H), 3.52-3.65 (m, 2H), 3.89-4.01 (m, 1H), 4.17-4.28 (m, 1H), 4.33 (q, J=7.03 Hz, 2H), 6.48 (d, J=1.76 Hz, 1H), 7.42 (s, 1H), 7.93 (d, J=8.03 Hz, 1H), 8.18 (t, J=7.91 Hz, 1H), 8.51 (d, J=7.78 Hz, 1H), 8.87 (d, J=1.51 Hz, 1H), 9.60 (s, 1H)

### Example 289: N-(8-(2,2-difluoroethoxy)-2-(tetrahydro-2H-pyran-4-yl)imidazo[1,2-a]pyridin-6-yl)-6-(difluoromethyl)picolinamide

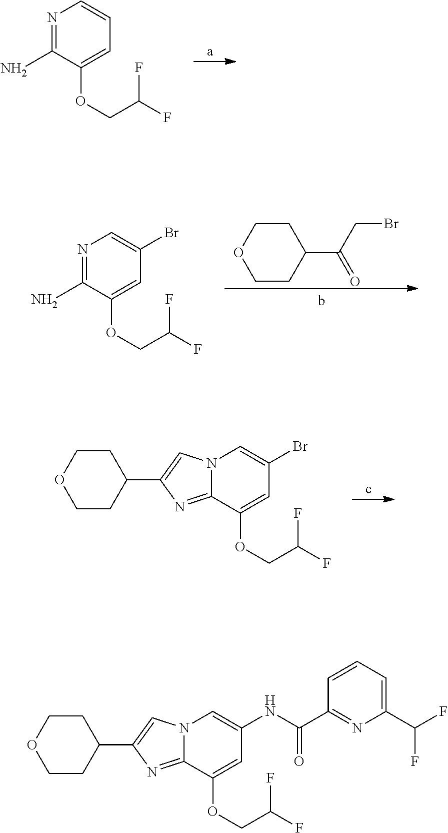 [SMILES: Nc1ncccc1OCC(F)F ; Nc1ncc(Br)cc1OCC(F)F ; O=C(CBr)C1CCOCC1 ; FC(F)COc1cc(Br)cn2cc(C3CCOCC3)nc12 ; O=C(Nc1cc(OCC(F)F)c2nc(C3CCOCC3)cn2c1)c1cccc(C(F)F)n1]

Step a: 1-Bromopyrrolidine-2,5-dione (511 mg, 2.87 mmol) was added to a mixture of 3-(2,2-difluoroethoxy)pyridin-2-amine (500 mg, 2.87 mmol) in MeCN (8.20 mL) at 0° C. and the reaction stirred at rt for 2 h. The reaction was quenched with aq. sat. NaHCO₃, extracted with EtOAc (3×), the combined organic extracts washed with brine, dried over MgSO₄, filtered and evaporated under reduced pressure to give 5-bromo-3-(2,2-difluoroethoxy)pyridin-2-amine (620 mg, 85.37% yield), that was used without additional purification. LCMS (ESI) m/z 255.1 (M+H)⁺;

Step b: A mixture of 5-bromo-3-(2,2-difluoroethoxy)pyridin-2-amine (250 mg, 987.99 μmol), 2-bromo-1-(tetrahydro-2H-pyran-4-yl)ethan-1-one (163.66 mg, 790.39 μmol) and NaHCO₃ (249 mg, 2.96 mmol) in MeCN (1.69 mL) and toluene (1.13 mL) was heated at 90° C. overnight. The reaction was cooled to rt, silica gel added and the mixture concentrated in vacuo. The solid was purified by dry load column chromatography on silica gel (0-100% 3:1 EtOAc/EtOH in Hept), to give 6-bromo-8-(2,2-difluoroethoxy)-2-tetrahydropyran-4-yl-imidazo[1,2-a]pyridine (70 mg, 19.62% yield). LCMS (ESI) m/z 363.1 (M+H)⁺;

Step c: A mixture of 6-bromo-8-(2,2-difluoroethoxy)-2-tetrahydropyran-4-yl-imidazo[1,2-a]pyridine (70 mg, 193.81 μmol), XantPhos (44.86 mg, 77.52 μmol), Cs₂CO₃ (126.29 mg, 387.62 μmol), Pd(OAc)₂ (8.70 mg, 38.76 μmol) and 6-(difluoromethyl)picolinamide (100.08 mg, 581.43 μmol) was purged with N₂, dioxane (3.23 mL) added, the reaction vessel sealed and heated at 100° C. overnight. The reaction was cooled to rt, filtered, and the filtrate concentrated in vacuo. The crude product was purified by prep HPLC using XSelect CSH Prep C18 OBD 5 μm 50×100 mm column eluting with H₂O/(0.2% NH₄OH in MeCN 5 to 70%) over 12 min (flow rate: 50 mL/min) to give N-(8-(2,2-difluoroethoxy)-2-(tetrahydro-2H-pyran-4-yl)imidazo[1,2-a]pyridin-6-yl)-6-(difluoromethyl)picolinamide (24.70 mg, 54.60 μmol, 28.17% yield). ¹H NMR (400 MHz, CDCl₃) δ ppm 1.74-1.93 (m, 2H), 2.11 (br d, J=13.05 Hz, 2H), 3.15 (br t, J=11.80 Hz, 1H), 3.59 (td, J=11.73, 1.88 Hz, 2H), 4.09 (dd, J=11.55, 2.51 Hz, 2H), 4.57 (td, J=12.80, 4.27 Hz, 2H), 6.18-6.50 (m, 1H), 6.63-6.93 (m, 1H), 6.72 (br s, 1H), 7.41 (s, 1H), 7.90 (d, J=7.03 Hz, 1H), 8.15 (t, J=7.91 Hz, 1H), 8.44 (dd, J=7.78, 0.75 Hz, 1H), 9.02 (s, 1H), 9.74 (s, 1H).

### Example 290: 6-(difluoromethyl)-N-(8-ethyl-2-(tetrahydro-2H-pyran-4-yl)imidazo[1,2-a]pyridin-6-yl)picolinamide

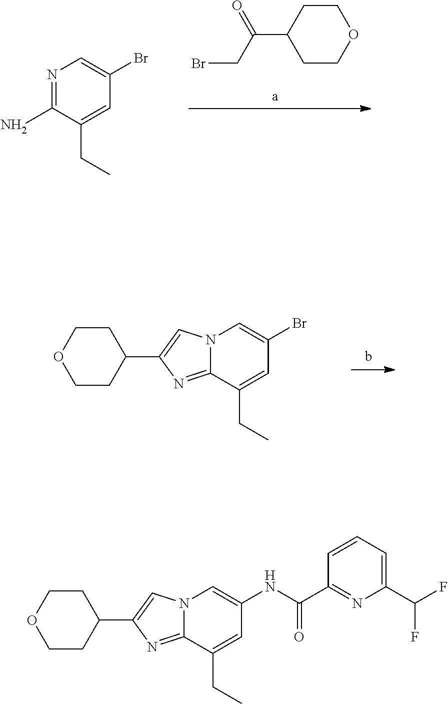

Step a: A mixture of 5-bromo-3-ethylpyridin-2-amine (200 mg, 994.73 μmol), 2-bromo-1-(tetrahydro-2H-pyran-4-yl)ethan-1-one (205.98 mg, 994.73 μmol) and NaHCO₃ (250.70 mg, 2.98 mmol) in MeCN (1.71 mL) and toluene (1.14 mL) was heated at 90° C. overnight. The reaction mixture was cooled to rt, filtered, silica gel added to the filtrate and the mixture concentrated in vacuo. The solid was purified by silica gel column chromatography to give 6-bromo-8-ethyl-2-tetrahydropyran-4-yl-imidazo[1,2-a]pyridine (230 mg, 74.78% yield). LCMS (ESI) m/z 311.1 (M+H)⁺;

Step b: A mixture of 6-bromo-8-ethyl-2-tetrahydropyran-4-yl-imidazo[1,2-a]pyridine (230 mg, 743.86 μmol), XantPhos (172.16 mg, 297.54 μmol), Cs₂CO₃ (484.73 mg, 1.49 mmol), Pd(OAc)₂ (33.40 mg, 148.77 μmol) and 6-(difluoromethyl)picolinamide (384.12 mg, 2.23 mmol) was purged with N₂, dioxane (4.96 mL) added, the reaction vessel sealed and heated at rt and heated at 100° C. overnight. The reaction mixture was cooled to rt, filtered, concentrated in vacuo, and purified by TFA-modified mass directed HPLC to give 6-(difluoromethyl)-N-(8-ethyl-2-(tetrahydro-2H-pyran-4-yl)imidazo[1,2-a]pyridin-6-yl)picolinamide 2,2,2-trifluoroacetate (64 mg, 21.49% yield). LCMS (ESI) m/z 401.3 (M+H)⁺; ¹H NMR (400 MHz, CDCl₃) δ ppm 1.46 (t, J=7.40 Hz, 3H), 1.76-1.91 (m, 2H), 2.10-2.21 (m, 2H), 3.15 (q, J=7.45 Hz, 2H), 3.28-3.38 (m, 1H), 3.63 (td, J=11.92, 1.76 Hz, 2H), 4.13 (dd, J=11.80, 3.26 Hz, 1H), 4.04-4.18 (m, 1H), 6.65-6.96 (m, 1H), 7.49 (s, 2H), 7.96 (d, J=7.03 Hz, 1H), 8.20 (t, J=7.91 Hz, 1H), 8.39-8.48 (m, 1H), 9.57 (d, J=1.51 Hz, 1H), 10.00 (s, 1H).

### Example 291: 1-(2,2-difluoroethyl)-N-(2-(1-(2,2-difluoroethyl)azetidin-3-yl)-7-ethoxyimidazo[1,2-a]pyridin-6-yl)-1H-pyrazole-3-carboxamide

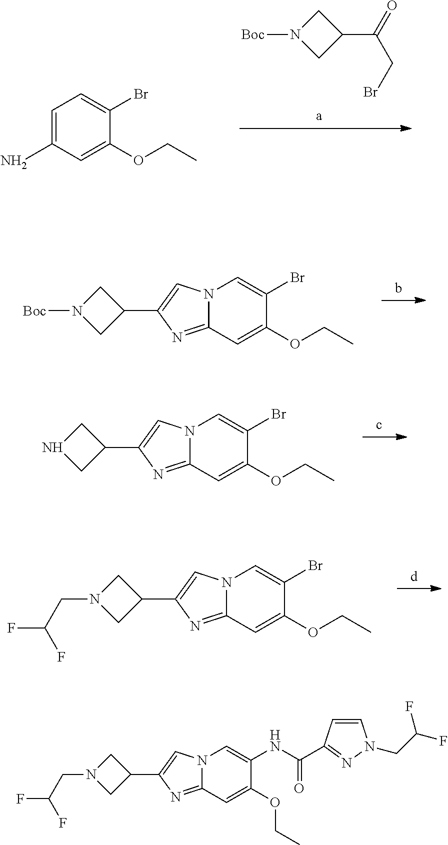 [SMILES: CC(C)(C)OC(=O)N1CC(C(=O)CBr)C1 ; CCOc1cc(N)ccc1Br ; CCOc1cc2nc(C3CN(C(=O)OC(C)(C)C)C3)cn2cc1Br ; CCOc1cc2nc(C3CNC3)cn2cc1Br ; CCOc1cc2nc(C3CN(CC(F)F)C3)cn2cc1Br ; CCOc1cc2nc(C3CN(CC(F)F)C3)cn2cc1NC(=O)c1ccn(CC(F)F)n1]

Step a: A mixture of 5-bromo-4-ethoxypyridin-2-amine (868.24 mg, 4.00 mmol), tert-butyl 3-(2-bromoacetyl)azetidine-1-carboxylate (1.0 g, 3.60 mmol) and NaHCO₃ (1.01 g, 12.0 mmol) in EtOH (10 mL) was heated at 80° C. overnight. The reaction was cooled to rt, silica gel added, the mixture concentrated in vacuo and purified by silica gel chromatography to obtain tert-butyl 3-(6-bromo-7-ethoxyimidazo[1,2-a]pyridin-2-yl)azetidine-1-carboxylate (1.00 g, 63.09% yield). LCMS (ESI) m/z 398.1 (M+H)⁺;

Step b: Tert-butyl 3-(6-bromo-7-ethoxyimidazo[1,2-a]pyridin-2-yl)azetidine-1-carboxylate (1.00 g, 2.52 mmol) was dissolved in HCl (4 M, 3.15 mL) and the solution stirred at rt for 2 h. The reaction was evaporated under reduced pressure to give 2-(azetidin-3-yl)-6-bromo-7-ethoxyimidazo[1,2-a]pyridine hydrochloride. LCMS (ESI) m/z 296.0 (M+H)⁺;

Step c: 60% NaH (220 mg, 5.50 mmol) was added to a solution of 2-(azetidin-3-yl)-6-bromo-7-ethoxyimidazo[1,2-a]pyridine hydrochloride (831.55 mg, 2.50 mmol) in THF (6.25 mL) and the solution stirred for 30 mins. 2,2-Difluoroethyl trifluoromethanesulfonate (802.91 mg, 3.75 mmol) was added and the reaction stirred overnight at rt. The solution was quenched with aq. NH₄Cl, extracted with EtOAc, dried and concentrated in vacuo. The crude product was purified by column chromatography on silica gel to give 6-bromo-2-(1-(2,2-difluoroethyl)azetidin-3-yl)-7-ethoxyimidazo[1,2-a]pyridine (170 mg, 18.88% yield). LCMS (ESI) m/z 360.1 (M+H)⁺;

Step d: A mixture of 6-bromo-2-(1-(2,2-difluoroethyl)azetidin-3-yl)-7-ethoxy-imidazo[1,2-a]pyridine (50 mg, 138.81 μmol), Pd(OAc)₂ (6.23 mg, 27.76 μmol), XantPhos (32.13 mg, 55.52 μmol), Cs₂CO₃ (90.46 mg, 277.62 μmol) and 1-(2,2-difluoroethyl)pyrazole-3-carboxamide (60.78 mg, 347.03 μmol) was purged with N₂, dioxane (1.39 mL) added, the reaction vessel sealed and the reaction heated at 80° C. overnight. The cooled reaction mixture was filtered through Celite® and the filtrate concentrated in vacuo. The residue was purified by reverse phase HPLC, to give 1-(2,2-difluoroethyl)-N-(2-(1-(2,2-difluoroethyl)azetidin-3-yl)-7-ethoxyimidazo[1,2-a]pyridin-6-yl)-1H-pyrazole-3-carboxamide (6.20 mg, 13.64 μmol, 9.83% yield). LCMS (ESI) m/z 455.2 (M+H)⁺; ¹H NMR (500 MHz, DMSO-d₆) δ ppm 1.44 (t, J=6.71 Hz, 3H), 2.85 (td, J=16.18, 4.27 Hz, 3H), 4.22 (d, J=7.32 Hz, 3H), 4.78 (td, J=15.26, 3.05 Hz, 3H), 5.82-6.14 (m, 1H), 6.30-6.55 (m, 1H), 6.86 (d, J=2.44 Hz, 1H), 7.06 (s, 1H), 7.71 (s, 1H), 7.99 (s, 1H), 9.16-9.30 (m, 2H)

### Example 292: 6-(difluoromethyl)-N-(7-(2-hydroxypropan-2-yl)-2-(tetrahydro-2H-pyran-4-yl)imidazo[1,2-a]pyridin-6-yl)picolinamide

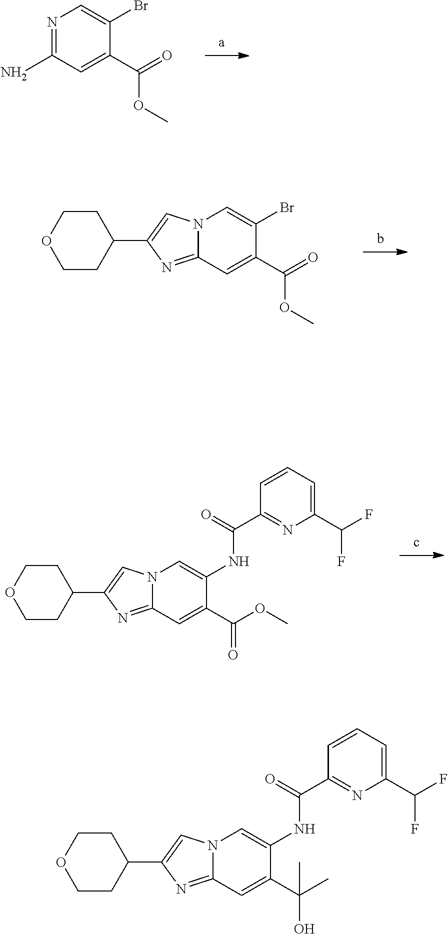 [SMILES: COC(=O)c1cc(N)ncc1Br ; COC(=O)c1cc2nc(C3CCOCC3)cn2cc1Br ; COC(=O)c1cc2nc(C3CCOCC3)cn2cc1NC(=O)c1cccc(C(F)F)n1 ; CC(C)(O)c1cc2nc(C3CCOCC3)cn2cc1NC(=O)c1cccc(C(F)F)n1]

Step a: A mixture of methyl 2-amino-5-bromopyridine-4-carboxylate (400 mg, 1.73 mmol), 2-bromo-1-tetrahydropyran-4-yl-ethan-1-one (358.21 mg, 1.73 mmol) and NaHCO₃ (290.67 mg, 3.46 mmol) in MeCN (3.46 mL) and toluene (2.31 mL) was heated at 90° C. overnight. The reaction was cooled to rt, silica gel added and the mixture concentrated in vacuo. The solid was purified by column chromatography on silica gel via dry load (0-100% 3:1 EtOAC/EtOH in Hept), to give methyl 6-bromo-2-(tetrahydro-2H-pyran-4-yl)imidazo[1,2-a]pyridine-7-carboxylate (350 mg, 59.65% yield). LCMS (ESI) m/z 341.1 (M+H)⁺;

Step b: A mixture of methyl 6-bromo-2-(tetrahydro-2H-pyran-4-yl)imidazo[1,2-a]pyridine-7-carboxylate (349.37 mg, 1.03 mmol), XantPhos (238.39 mg, 412 μmol), Cs₂CO₃ (671.19 mg, 2.06 mmol), Pd(OAc)₂ (46.25 mg, 206.00 μmol) and 6-(difluoromethyl)picolinamide (531.88 mg, 3.09 mmol) was purged with N₂, dioxane (6.87 mL) added, the reaction vessel sealed and heated at 100° C. overnight. The reaction was cooled to rt, filtered, silica gel added and the mixture concentrated in vacuo. The solid was purified by column chromatography on silica gel via dry load (0-100% 3:1 EtOAc/EtOH in Heptanes) to give methyl 6-(6-(difluoromethyl)picolinamido)-2-(tetrahydro-2H-pyran-4-yl)imidazo[1,2-a]pyridine-7-carboxylate (210.00 mg, 487.92 μmol, 47.37% yield), LCMS (ESI) m/z 431.3 (M+H)⁺.

Step c: MeMgBr (3 M, 774.47 uL) was added dropwise to a solution of methyl 6-(6-(difluoromethyl)picolinamido)-2-(tetrahydro-2H-pyran-4-yl)imidazo[1,2-a]pyridine-7-carboxylate (100 mg, 232.34 μmol) in THF (2.32 mL) at 0° C. and the reaction stirred for 3 h. The reaction was quenched with aq. NH₄Cl, extracted with EtOAc (3×), the combined organic extracts dried over MgSO₄, filtered, and concentrated in vacuo. The crude product was purified by TFA-modified mass directed HPLC to give 6-(difluoromethyl)-N-(7-(2-hydroxypropan-2-yl)-2-(tetrahydro-2H-pyran-4-yl)imidazo[1,2-a]pyridin-6-yl)picolinamide 2,2,2-trifluoroacetate (65.60 mg, 49.78% yield). LCMS (ESI) m/z 431.3 (M+H)⁺; ¹H NMR (400 MHz, CDCl₃) δ ppm 1.72 (br d, J=4.27 Hz, 1H), 1.61-1.70 (m, 6H), 1.64-1.72 (m, 1H), 1.86 (br d, J=12.30 Hz, 2H), 2.81-2.87 (m, 1H), 3.29 (br t, J=11.55 Hz, 2H), 3.81-3.92 (m, 3H), 6.50-6.82 (m, 1H), 7.17 (s, 1H), 7.33 (s, 1H), 7.76 (d, J=7.53 Hz, 1H), 8.01 (t, J=7.78 Hz, 1H), 8.29 (d, J=7.53 Hz, 1H), 9.40 (s, 1H), 12.06 (s, 1H).

### Example 293: 6-(difluoromethyl)-N-(7-fluoro-2-(tetrahydro-2H-pyran-4-yl)imidazo[1,2-a]pyridin-6-yl)picolinamide

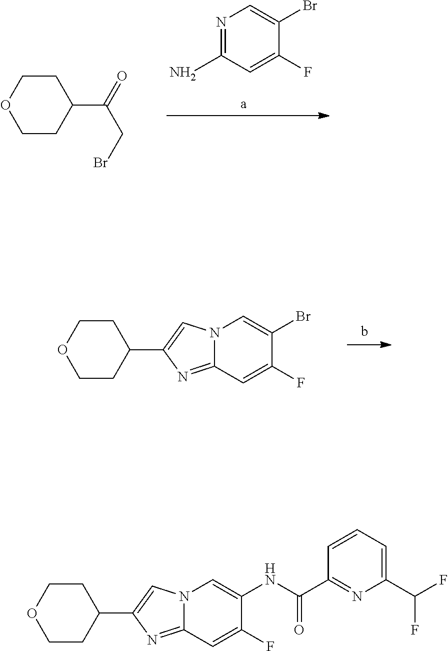 [SMILES: Nc1cc(F)c(Br)cn1 ; O=C(CBr)C1CCOCC1 ; Fc1cc2nc(C3CCOCC3)cn2cc1Br ; O=C(Nc1cn2cc(C3CCOCC3)nc2cc1F)c1cccc(C(F)F)n1]

Step a: A mixture of 5-bromo-4-fluoropyridin-2-amine (500 mg, 2.62 mmol), 2-bromo-1-(tetrahydro-2H-pyran-4-yl)ethan-1-one (542.04 mg, 2.62 mmol) in MeCN (9.83 mL) and toluene (3.28 mL) was heated at 100° C. overnight. The reaction was cooled to rt, silica gel was added and the mixture concentrated in vacuo. The product was purified by column chromatography to give 6-bromo-7-fluoro-2-(tetrahydro-2H-pyran-4-yl)imidazo[1,2-a]pyridine (520 mg, 66.35% yield). LCMS (ESI) m/z 381.3 (M+H)⁺;

Step b: A mixture of 6-bromo-7-fluoro-2-(tetrahydro-2H-pyran-4-yl)imidazo[1,2-a]pyridine (520 mg, 1.74 mmol), Pd(OAc)₂ (78.13 mg, 348 μmol), XantPhos (402.33 mg, 696 μmol), Cs₂CO₃ (1.13 g, 3.48 mmol) and 6-(difluoromethyl)picolinamide (748.04 mg, 4.35 mmol) was purged with N₂, dioxane (17.40 mL) added and the reaction vessel sealed and heated at 100° C. overnight. The reaction mixture was cooled to rt, filtered through Celite®, and the filtrate concentrated in vacuo. The residue was purified by mass directed HPLC, to give 6-(difluoromethyl)-N-(7-fluoro-2-(tetrahydro-2H-pyran-4-yl)imidazo[1,2-a]pyridin-6-yl)picolinamide (32.50 mg, 4.78% yield). ¹H NMR (400 MHz, METHANOL-d₄) δ ppm: 1.78-2.10 (m, 3H), 3.02 (s, 1H), 3.53-3.69 (m, 2H), 4.06 (br d, J=11.29 Hz, 2H), 6.71-7.06 (m, 1H), 7.36 (d, J=11.04 Hz, 1H), 7.71 (s, 1H), 7.93-8.07 (m, 1H), 8.27 (s, 1H), 8.39 (d, J=1.00 Hz, 1H), 9.35 (d, J=7.28 Hz, 1H)

### Example 294: N-(2-chloro-7-isopropoxyimidazo[1,2-a]pyridin-6-yl)-6-(difluoromethyl)picolinamide

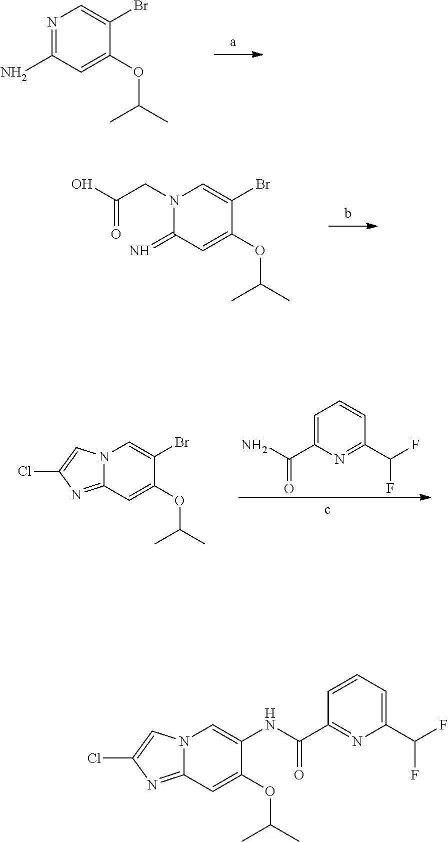 [SMILES: CC(C)Oc1cc(N)ncc1Br ; CC(C)Oc1cc(=N)n(CC(=O)O)cc1Br ; CC(C)Oc1cc2nc(Cl)cn2cc1Br ; NC(=O)c1cccc(C(F)F)n1 ; CC(C)Oc1cc2nc(Cl)cn2cc1NC(=O)c1cccc(C(F)F)n1]

Step a: TEA (117.99 mg, 1.17 mmol) was added dropwise to a mixture of 2-chloroacetic acid (100.17 mg, 1.06 mmol) and water (1.06 mL) and the solution stirred for 10 mins. 5-Bromo-4-isopropoxypyridin-2-amine (293.95 mg, 1.27 mmol) was added and the reaction mixture heated at 90° C. for 2 h. The reaction was cooled to rt, EtOH was added and the mixture stirred at 0° C. for 30 mins. The resulting mixture was filtered and the solid dried to give 2-(5-bromo-2-imino-4-isopropoxypyridin-1(2H)-yl)acetic acid. LCMS (ESI) m/z 290.0 (M+H)⁺;

Step b: Phosphorus (V) oxide chloride (58.50 uL, 629.48 μmol) was added to a solution of 2-(5-bromo-2-imino-4-isopropoxypyridin-1(2H)-yl)acetic acid (91.00 mg, 314.74 μmol) in toluene (3 mL) and the mixture was warmed to 120° C. for 2 h under microwave irradiation. The cooled mixture was poured onto ice water, stirred for 10 mins and the phases separated. The aqueous phase was neutralised with 1N NaOH, extracted with EtOAc, dried over Na₂SO₄ filtered and concentrated in vacuo. The crude was purified by column chromatography on silica gel (0-100% EtOAc/Heptane) to afford 6-bromo-2-chloro-7-isopropoxyimidazo[1,2-a]pyridine. LCMS (ESI) m/z 290.8 (M+H)⁺;

Step c: A mixture of 6-bromo-2-chloro-7-isopropoxyimidazo[1,2-a]pyridine (60 mg, 207.21 μmol), 6-(difluoromethyl)picolinamide (37 mg, 214.95 μmol), XantPhos-Pd-G3 (19.65 mg, 20.72 μmol) and Cs₂CO₃ (135.03 mg, 414.42 μmol) in toluene (2.07 ml) was heated at 90° C. overnight. The cooled mixture was concentrated in vacuo, the residue suspended in MeOH and filtered through PTFE and purified by reverse phase HPLC eluting with 0.1% aq. NH₄OH (0.1% aq. NH₄OH in MeCN 5-75%) to give N-(2-chloro-7-isopropoxyimidazo[1,2-a]pyridin-6-yl)-6-(difluoromethyl)picolinamide. LCMS (ESI) m/z 380.9 (M+H)⁺; ¹H NMR (500 MHz, DMSO-d₆) δ ppm: 1.43 (d, J=6.10 Hz, 7H), 4.93 (quin, J=6.03 Hz, 1H), 6.98-7.29 (m, 2H), 7.97-8.06 (m, 2H), 8.28-8.40 (m, 2H), 9.52 (s, 1H), 10.56 (s, 1H)

### Example 295: 6-(difluoromethyl)-N-(7-methoxy-8-methyl-2-(1-methyl-2-oxabicyclo[2.1.1]hexan-4-yl)imidazo[1,2-a]pyridin-6-yl)picolinamide

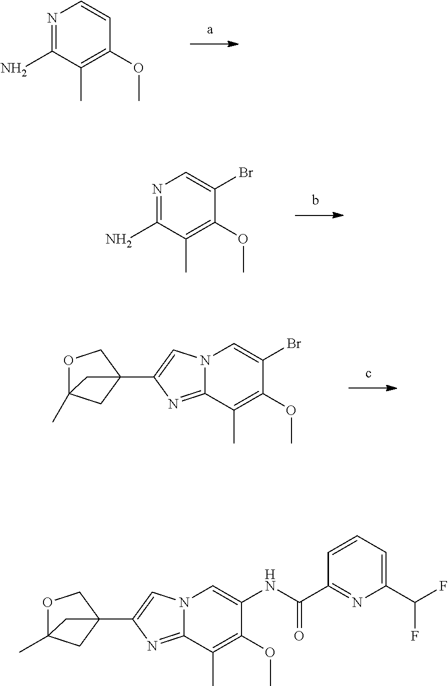

6-(Difluoromethyl)-N-(7-methoxy-8-methyl-2-(1-methyl-2-oxabicyclo[2.1.1]hexan-4-yl)imidazo[1,2-a]pyridin-6-yl)picolinamide 2,2,2-trifluoroacetate was prepared following the same synthetic sequence described in Example 289, except 4-methoxy-3-methylpyridin-2-amine was used instead of 3-(2,2-difluoroethoxy)pyridin-2-amine and 2-bromo-1-(1-methyl-2-oxabicyclo[2.1.1]hexan-4-yl)ethan-1-one was used instead of 2-bromo-1-(tetrahydro-2H-pyran-4-yl)ethan-1-one. (38.50 mg, 33.67% yield) LCMS (ESI) m/z 429.3 (M+H)⁺; ¹H NMR (400 MHz, CDCl₃) δ ppm 2.00-2.09 (m, 2H), 2.27-2.34 (m, 2H), 2.69 (s, 3H), 4.10-4.14 (m, 4H), 6.60-6.91 (m, 1H), 7.43 (s, 1H), 7.95 (d, J=7.03 Hz, 1H), 8.19 (t, J=7.78 Hz, 1H), 8.41-8.46 (m, 1H), 9.66 (s, 1H), 10.62 (s, 1H).

### Example 296: 6-(difluoromethyl)-N-(8-methyl-2-(tetrahydro-2H-pyran-4-yl)imidazo[1,2-a]pyrazin-6-yl)picolinamide

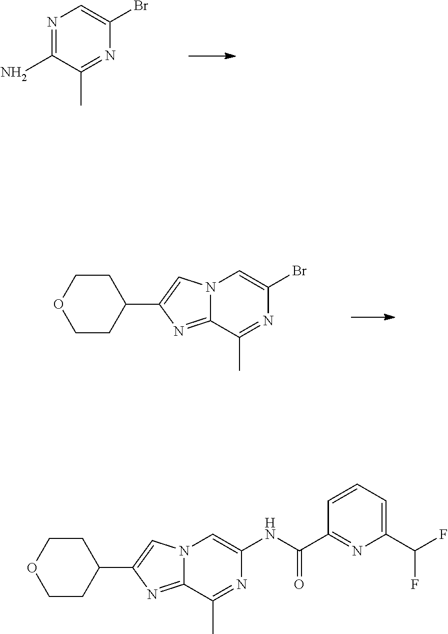

Step a: A mixture of 5-bromo-3-methylpyrazin-2-amine (300.85 mg, 1.60 mmol), 2-bromo-1-(tetrahydro-2H-pyran-4-yl)ethan-1-one (331.30 mg, 1.60 mmol) and NaHCO₃ (403.25 mg, 4.80 mmol) in EtOH (6.40 mL) was heated at 80° C. overnight. Further 2-bromo-1-(tetrahydro-2H-pyran-4-yl)ethan-1-one (331.30 mg, 1.60 mmol) and toluene (2 mL) were added and the reaction heated at 95° C. for 6 h. The reaction was cooled to rt, silica gel added, the mixture concentrated in vacuo and purified via dry load (12 g, 0-50% 3:1 EtOAc/EtOH in Hept) to give 6-bromo-8-methyl-2-tetrahydropyran-4-yl-imidazo[1,2-a]pyrazine (140 mg, 29.54% yield). LCMS (ESI) m/z 295.9 (M+H)⁺;

Step b: 6-(difluoromethyl)-N-(8-methyl-2-(tetrahydro-2H-pyran-4-yl)imidazo[1,2-a]pyrazin-6-yl)picolinamide was prepared from give 6-bromo-8-methyl-2-tetrahydropyran-4-yl-imidazo[1,2-a]pyrazine, following a similar method to that described in Example 281 (12.70 mg, 13.87% yield), LCMS (ESI) m/z 388.2 (M+H)⁺; ¹H NMR (500 MHz, DMSO-d₆) δ ppm 1.69-1.81 (m, 2H) 1.95 (br dd, J=12.82, 2.44 Hz, 2H) 2.75 (s, 3H) 3.04 (tt, J=11.60, 3.97 Hz, 1H) 3.49 (td, J=11.60, 1.83 Hz, 1H) 3.95 (dt, J=9.46, 2.29 Hz, 2H) 7.06-7.33 (m, 1H) 8.04 (dd, J=7.02, 1.53 Hz, 1H) 8.15 (s, 1H) 8.30-8.39 (m, 2H) 9.27 (s, 1H) 10.22 (s, 1H)

### Example 297 and 298: 6-(difluoromethyl)-N-(8-methoxy-2-(tetrahydro-2H-pyran-3-yl)imidazo[1,2-a]pyrazin-6-yl)picolinamide, Enantiomer 1 and 2

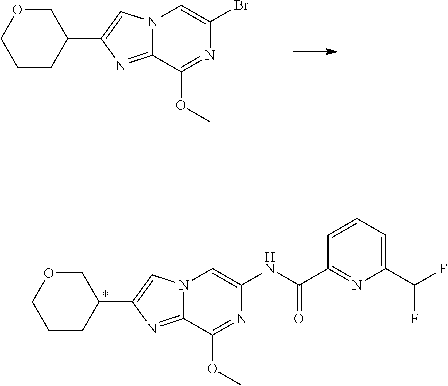 [SMILES: COc1nc(Br)cn2cc(C3CCCOC3)nc12 ; COc1nc(NC(=O)c2cccc(C(F)F)n2)cn2cc(C3CCCOC3)nc12]

6-(difluoromethyl)-N-(8-methoxy-2-(tetrahydro-2H-pyran-3-yl)imidazo[1,2-a]pyrazin-6-yl)picolinamide was prepared from 5-bromo-3-methoxypyrazin-2-amine, 2-bromo-1-(tetrahydro-2H-pyran-3-yl)ethan-1-one and 6-(difluoromethyl)picolinamide, following a similar method to that described in Example 285 and 286. LCMS (ESI) m/z 404.3 (M+H)⁺; The compound was further purified by SFC using a CHIRALPAK AD-H 30×250 mm, 5 μm column, 40% MeOH w/0.1% DEA in CO2 (flow rate: 100 mL/min, ABPR 120 bar, MBPR 40 psi, column temp 40° C.) to provide enantiomer 1, ¹H NMR (400 MHz, CDCl₃ δ ppm 1.71-1.81 (m, 2H), 1.89-1.98 (m, 1H), 2.14-2.20 (m, 1H), 3.05-3.17 (m, 1H), 3.49 (d, J=5.52 Hz, 1H), 3.50-3.57 (m, 1H), 3.64 (dd, J=11.04, 9.79 Hz, 1H), 3.95 (dt, J=10.92, 3.45 Hz, 1H), 4.16 (ddd, J=11.04, 4.14, 1.88 Hz, 1H), 4.19 (s, 3H), 6.62-6.95 (m, 1H), 7.49 (s, 1H), 7.83-7.88 (m, 1H), 8.11 (t, J=7.78 Hz, 1H), 8.41 (dd, J=7.78, 0.75 Hz, 1H), 8.94 (s, 1H), 9.85 (s, 1H)

Further Elution Provided Enantiomer 2

¹H NMR (400 MHz, CDCl₃) δ ppm 1.74-1.78 (m, 1H), 1.90-1.97 (m, 1H), 2.18 (br dd, J=12.80, 4.02 Hz, 1H), 3.10-3.17 (m, 1H), 3.49 (d, J=5.52 Hz, 1H), 3.51-3.58 (m, 1H), 3.64 (dd, J=11.04, 9.79 Hz, 1H), 3.89-3.98 (m, 1H), 4.14-4.18 (m, 1H), 4.20 (s, 3H), 6.62-6.94 (m, 1H), 7.49 (s, 1H), 7.88 (d, J=7.78 Hz, 1H), 8.12 (t, J=7.91 Hz, 1H), 8.41 (dd, J=7.78, 0.75 Hz, 1H), 8.94 (s, 1H), 9.85 (s, 1H).

### Example 299 and 300: N-(8-methoxy-2-(tetrahydro-2H-pyran-3-yl)imidazo[1,2-a]pyrazin-6-yl)-6-(trifluoromethyl)picolinamide, enantiomer 1 and 2

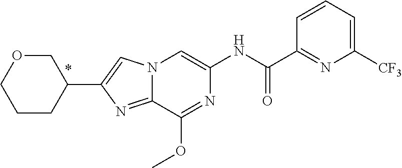 [SMILES: COc1nc(NC(=O)c2cccc(C(F)(F)F)n2)cn2cc(C3CCCOC3)nc12]

N-(8-methoxy-2-(tetrahydro-2H-pyran-3-yl)imidazo[1,2-a]pyrazin-6-yl)-6-(trifluoromethyl)picolinamide, enantiomer 1 and 2 were prepared from 5-bromo-3-methoxypyrazin-2-amine, 2-bromo-1-(tetrahydro-2H-pyran-3-yl)ethan-1-one and 6-(trifluoromethyl)picolinamide, following a similar method to that described in Example 285 and 286. LCMS (ESI) m/z 422.2 (M+H)⁺; The enantiomers were separated by SFC using a CHIRALPAK IB 30×250 mm, 5 μm column, eluting with 45% EtOH w/0.1% DEA in CO₂ (flow rate: 100 mL/min, ABPR 120 bar, MBPR 40 psi, column temp 40° C.) to provide enantiomer 1 ¹H NMR (400 MHz, METHANOL-d₄) δ ppm 1.74-1.82 (m, 2H), 1.86-1.95 (m, 1H), 2.15-2.23 (m, 1H), 3.04-3.13 (m, 1H), 3.53-3.62 (m, 2H), 3.91-3.99 (m, 1H), 4.09-4.17 (m, 1H), 4.22 (s, 3H), 7.88 (s, 1H), 8.10 (dd, J=7.78, 1.00 Hz, 1H), 8.34 (t, J=7.91 Hz, 1H), 8.51 (d, J=7.78 Hz, 1H), 9.05 (s, 1H).

Further Elution Provided Enantiomer 2.

¹H NMR (400 MHz, METHANOL-d₄) δ ppm 1.71-1.82 (m, 2H), 1.86-1.96 (m, 1H), 2.20 (br dd, J=12.93, 3.89 Hz, 1H), 3.01-3.12 (m, 1H), 3.53-3.62 (m, 2H), 3.92-3.97 (m, 1H), 4.10-4.17 (m, 1H), 4.22 (s, 3H), 7.88 (s, 1H), 8.11 (dd, J=7.78, 1.00 Hz, 1H), 8.29-8.37 (m, 1H), 8.51 (d, J=7.78 Hz, 1H), 9.05 (s, 1H).

### Example 301 and 302: N-(8-(difluoromethoxy)-2-(tetrahydro-2H-pyran-3-yl)imidazo[1,2-a]pyrazin-6-yl)-6-(difluoromethyl)picolinamide, enantiomer 1 and

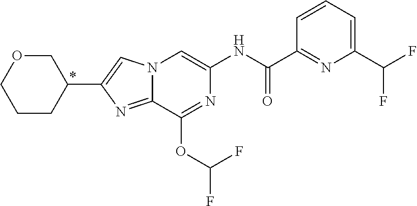 [SMILES: O=C(Nc1cn2cc(C3CCCOC3)nc2c(OC(F)F)n1)c1cccc(C(F)F)n1]

N-(8-(difluoromethoxy)-2-(tetrahydro-2H-pyran-3-yl)imidazo[1,2-a]pyrazin-6-yl)-6-(difluoromethyl)picolinamide was prepared from 2-amino-3-(difluoromethoxy)pyrazine, 2-bromo-1-(tetrahydro-2H-pyran-3-yl)ethan-1-one and 6-(difluoromethyl)picolinamide following the same synthetic sequence as described in Example 295. LCMS (ESI) m/z 439.2 (M+H)⁺; The product was further purified by SFC using a LUX Cellulose-4 LC 4.6×150 mm, 3 μm column, eluting with 40% MeOH w/0.1% DEA in CO2 (flow rate: 100 mL/min, ABPR 120 bar, MBPR 40 psi, column temp 40° C.) to give enantiomer 1: ¹H NMR (400 MHz, CDCl₃) δ ppm 1.74-1.94 (m, 3H), 2.22 (br d, J=9.29 Hz, 1H), 3.09-3.20 (m, 1H), 3.51-3.66 (m, 2H), 3.93-4.00 (m, 1H), 4.20 (dd, J=11.04, 2.76 Hz, 1H), 6.63-6.92 (m, 1H), 6.96 (d, J=1.51 Hz, 1H), 7.33-7.71 (m, 2H), 7.90 (d, J=7.28 Hz, 1H), 8.15 (t, J=7.78 Hz, 1H), 8.44 (d, J=7.78 Hz, 1H), 9.21 (d, J=1.51 Hz, 1H), 9.69 (s, 1H)

Further elution gave enantiomer 2: ¹H NMR (400 MHz, CDCl₃) δ ppm 1.64-1.76 (m, 2H), 2.00-2.09 (m, 1H), 2.18-2.26 (m, 1H), 3.32-3.40 (m, 1H), 3.75-3.84 (m, 2H), 3.90 (dd, J=11.17, 6.15 Hz, 1H), 4.06 (dd, J=11.55, 3.51 Hz, 1H), 6.66-7.11 (m, 2H), 7.63 (s, 1H), 7.79 (s, 1H), 7.96 (d, J=7.78 Hz, 1H), 8.19 (t, J=7.78 Hz, 1H), 8.44 (d, J=7.28 Hz, 1H), 9.56 (d, J=1.76 Hz, 1H), 10.11 (br s, 1H).

### Example 303: 6-(difluoromethyl)-N-(2-(tetrahydro-2H-pyran-4-yl)-8-(2,2,2-trifluoroethoxy)imidazo[1,2-a]pyrazin-6-yl)picolinamide

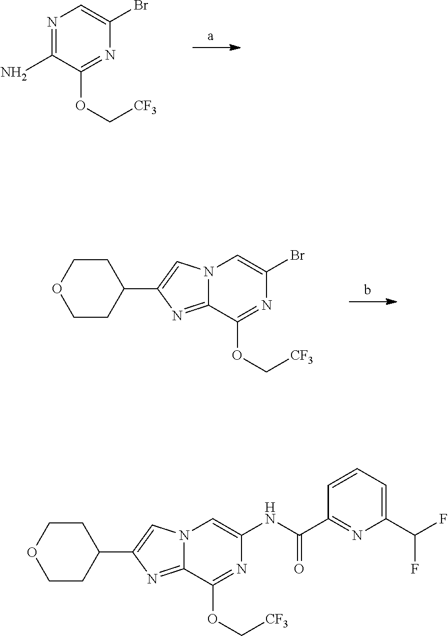 [SMILES: Nc1ncc(Br)nc1OCC(F)(F)F ; FC(F)(F)COc1nc(Br)cn2cc(C3CCOCC3)nc12 ; O=C(Nc1cn2cc(C3CCOCC3)nc2c(OCC(F)(F)F)n1)c1cccc(C(F)F)n1]

6-(Difluoromethyl)-N-(2-(tetrahydro-2H-pyran-4-yl)-8-(2,2,2-trifluoroethoxy)imidazo[1,2-a]pyrazin-6-yl)picolinamide 2,2,2-trifluoroacetate was prepared from 5-bromo-3-(2,2,2-trifluoroethoxy)pyrazin-2-amine, 2-bromo-1-(tetrahydro-2H-pyran-4-yl)ethan-1-one and 6-(difluoromethyl)picolinamide following the synthetic sequence described in Example 293. (8.50 mg, 18.03 μmol, 5.27% yield). LCMS (ESI) m/z 472.1 (M+H)⁺; ¹H NMR (400 MHz, CDCl₃) δ ppm 1.74-1.93 (m, 2H), 2.09 (br dd, J=12.92, 1.88 Hz, 2H), 3.26 (tt, J=11.86, 3.70 Hz, 1H), 3.62 (td, J=11.86, 1.88 Hz, 2H), 4.12 (dd, J=11.67, 2.89 Hz, 2H), 5.03 (q, J=8.11 Hz, 2H), 6.66-6.97 (m, 1H), 7.57 (s, 1H), 7.94 (d, J=7.78 Hz, 1H), 8.17 (t, J=7.91 Hz, 1H), 8.44 (dd, J=7.78, 0.75 Hz, 1H), 9.16 (s, 1H), 9.98 (s, 1H)

### Example 304: 6-(difluoromethyl)-N-(8-methoxy-2-(tetrahydro-2H-pyran-4-yl)imidazo[1,2-a]pyridin-6-yl)picolinamide

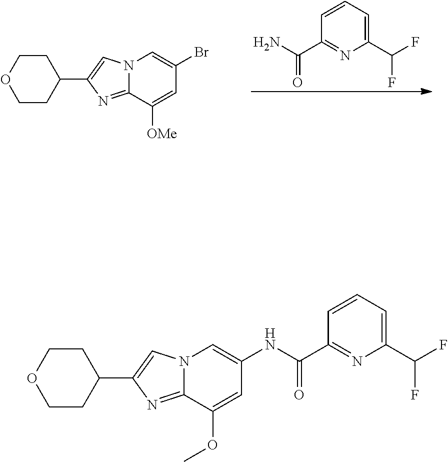 [SMILES: COc1cc(Br)cn2cc(C3CCOCC3)nc12 ; NC(=O)c1cccc(C(F)F)n1 ; COc1cc(NC(=O)c2cccc(C(F)F)n2)cn2cc(C3CCOCC3)nc12]

Pd₂(dba)₃ (5.54 mg, 0.0096 mmol), XantPhos (11.16 mg, 0.0193 mmol) and Cs₂CO₃ (62.82 mg, 0.193 mmol) were added to a solution of Example 281, step a: 6-bromo-8-methoxy-2-(tetrahydro-2H-pyran-4-yl)imidazo[1,2-a]pyridine (30 mg, 0.0964 mmol) and 6-(difluoromethyl)picolinamide (49.79 mg, 0.289 mmol) in toluene (5 mL) and the reaction purged with N₂. The reaction was stirred at 110° C. for 12 h, the mixture concentrated in vacuo and the residue purified by prep-HPLC eluting with MeCN:0.225% aq. formic acid to give 6-(difluoromethyl)-N-(8-methoxy-2-(tetrahydro-2H-pyran-4-yl)imidazo[1,2-a]pyridin-6-yl)picolinamide (1.50 mg, 0.373 mmol, 3.87% yield) as a brown solid. LCMS (ESI) m/z 403.0 (M+H)⁺; ¹H NMR (500 MHz, METHANOL-d₄) δ: 1.83-1.88 (m, 2H), 2.00-2.04 (m, 2H), 3.01-3.06 (m, 1H), 3.57-3.63 (m, 2H), 4.03-4.07 (m, 2H), 4.09 (s, 3H), 6.79-7.02 (m, 1H), 7.24 (s, 1H), 7.96 (d, J=6.4 Hz, 1H), 8.24 (t, J=6.4 Hz, 1H), 8.38 (d, J=6.4 Hz, 1H), 9.06 (s, 1H).

### Example 305: N-(8-methoxy-2-(tetrahydro-2H-pyran-4-yl)imidazo[1,2-a]pyridin-6-yl)-2-methylthiazole-4-carboxamide

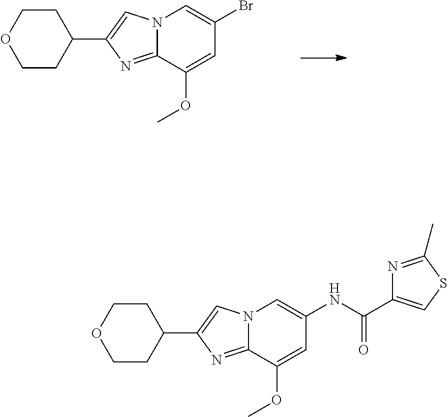 [SMILES: COc1cc(Br)cn2cc(C3CCOCC3)nc12 ; COc1cc(NC(=O)c2csc(C)n2)cn2cc(C3CCOCC3)nc12]

A mixture of 2-methylthiazole-4-carboxamide (58.49 mg, 411.35 μmol), Example 280, step a: 6-bromo-8-methoxy-2-(tetrahydro-2H-pyran-4-yl)imidazo[1,2-a]pyridine (80 mg, 257.09 μmol), iodocopper (29.38 mg, 154.26 μmol) and K₂CO₃ (106.60 mg, 771.28 μmol) was purged with N₂, dioxane (2.14 mL) and trans-N,N′-dimethylcyclohexane-1,2-diamine (21.94 mg, 154.26 μmol) were added, the reaction vessel sealed and heated at 100° C. for 24 h. The reaction was cooled to rt, filtered through Celite®, the filtrate concentrated in vacuo and the residue purified by TFA-modified mass directed HPLC to give N-(8-methoxy-2-(tetrahydro-2H-pyran-4-yl)imidazo[1,2-a]pyridin-6-yl)-2-methylthiazole-4-carboxamide 2,2,2-trifluoroacetate, 28.80 mg. LCMS (ESI) m/z 373.1 (M+H)⁺; ¹H NMR (400 MHz, METHANOL-d₄) δ ppm 1.71-1.77 (m, 2H), 1.94-1.97 (m, 2H), 2.80 (s, 3H), 3.08-3.11 (m, 1H), 3.45-3.49 (m, 2H), 3.94-3.97 (m, 2H), 4.08 (s, 3H), 7.89 (s, 1H), 8.31 (s, 1H), 9.32 (s, 1H), 10.73 (s, 1H).

### Example 306: N-(2-((1R*,5S*,6s*)-3-oxabicyclo[3.1.0]hexan-6-yl)-8-ethoxyimidazo[1,2-a]pyridin-6-yl)-6-(difluoromethyl)picolinamide

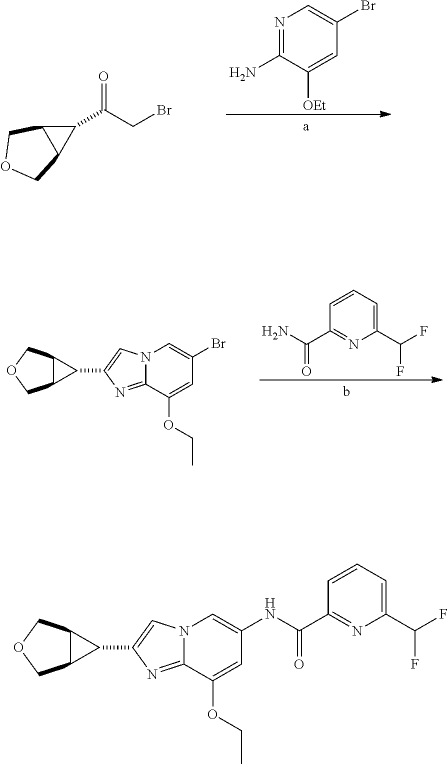

Step a: NaHCO₃ (58.06 mg, 0.691 mmol) was added to a solution of 5-bromo-3-ethoxypyridine-2-amine (50 mg, 0.231 mmol) and 1-rac-3-oxabicyclo[3.1.0]hexan-6-yl)-2-bromoethan-1-one (85.02 mg, 0.415 mmol) in EtOH (1 mL) and the reaction mixture stirred at 80° C. for 12 h. The reaction mixture was cooled to rt and concentrated in vacuo. The crude product was purified by column chromatography on silica gel eluting with EtOAc/DCM=1/3 to give 2-((1R,5S,6s)-3-oxabicyclo[3.1.0]hexan-6-yl)-6-bromo-8-ethoxyimidazo[1,2-a]pyridine (20 mg, 26.87% yield) as a yellow oil. LCMS (ESI) m/z 322.9 (M+H)⁺; ¹H NMR (500 MHz, CDCl₃) δ: 1.59 (t, J=7.0 Hz, 3H), 2.03 (br s, 1H), 3.81 (d, J=8.5 Hz, 1H), 4.02 (d, J=8.0 Hz, 1H), 4.24 (q, J=7.0 Hz, 2H), 6.50 (s, 1H), 7.25 (s, 1H), 7.80 (s, 1H).

Step b: 2-((1R,5S,6s)-3-oxabicyclo[3.1.0]hexan-6-yl)-6-bromo-8-ethoxyimidazo[1,2-a]pyridine (30 mg, 0.0964 mmol) and 6-(difluoromethyl)picolinamide (69.24 mg, 0.402 mmol) was dissolved in toluene (10 mL). Pd₂(dba)₃ (11.56 mg, 0.0201 mmol), XantPhos (23.27 mg, 0.0402 mmol) and Cs₂CO₃ (131.06 mg, 0.402 mmol) were added, the reaction mixture purged with N₂ and stirred at 110° C. for 12 h. The cooled mixture was concentrated in vacuo and the residue purified by prep-HPLC (Column: Welch Xtimate C18 150×25 mm×5 μm, eluting with water/(10 mM, NH₄HCO₃-MeCN from 33% to 63%), to give N-(2-((1R*,5S*,6s*)-3-oxabicyclo[3.1.0]hexan-6-yl)-8-ethoxyimidazo[1,2-a]pyridin-6-yl)-6-(difluoromethyl)picolinamide (45 mg, 53.45% yield) as a yellow solid. LCMS (ESI) m/z 415.0 (M+H)⁺; ¹H NMR (500 MHz, METHANOL-d₄) δ: 1.55 (t, J=7.0 Hz, 3H), 1.92 (t, J=3.5 Hz, 1H), 2.11 (brs, 2H), 3.80 (d, J=8.0 Hz, 2H), 3.99 (d, J=8.0 Hz, 2H), 4.27 (q, J=7.0 Hz, 2H), 6.77-7.00 (m, 2H), 7.57 (s, 1H), 7.92 (d, J=7.0 Hz, 1H), 8.21 (t, J=7.5 Hz, 1H), 8.33 (d, J=7.5 Hz, 1H), 8.87 (s, 1H).

### Example 307: N-(8-(difluoromethoxy)-2-(tetrahydro-2H-pyran-4-yl)imidazo[1,2-a]pyridin-6-yl)-6-(difluoromethyl)picolinamide

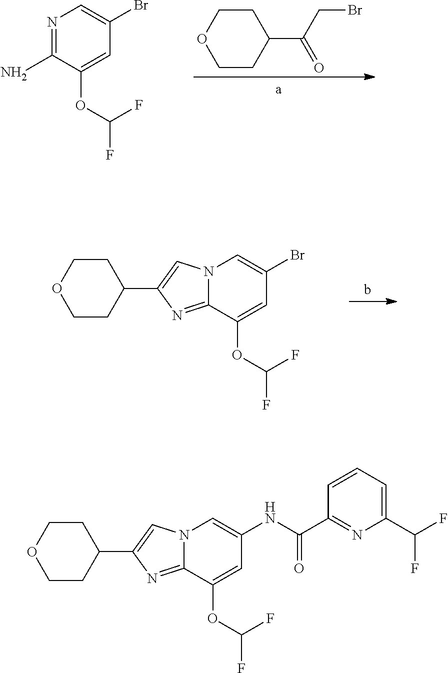 [SMILES: Nc1ncc(Br)cc1OC(F)F ; O=C(CBr)C1CCOCC1 ; FC(F)Oc1cc(Br)cn2cc(C3CCOCC3)nc12 ; O=C(Nc1cc(OC(F)F)c2nc(C3CCOCC3)cn2c1)c1cccc(C(F)F)n1]

Step a: A mixture of 5-bromo-3-(difluoromethoxy)pyridin-2-amine (399.16 mg, 1.67 mmol), 2-bromo-1-tetrahydropyran-4-yl-ethan-1-one (345.79 mg, 1.67 mmol) and NaHCO₃ (420.89 mg, 5.01 mmol) in MeCN (2.86 mL) was heated at 90° C. overnight. The reaction was cooled to rt, silica added, the mixture concentrated in vacuo and purified by column chromatography via dry load (0-50% 3:1 EtOAC/EtOH in Hept) to give 6-bromo-8-(difluoromethoxy)-2-(tetrahydro-2H-pyran-4-yl)imidazo[1,2-a]pyridine (480 mg, 82.80% yield). LCMS (ESI) m/z 349.0 (M+H)⁺;

Step b: A mixture of 6-bromo-8-(difluoromethoxy)-2-(tetrahydro-2H-pyran-4-yl)imidazo[1,2-a]pyridine (75 mg, 216.04 μmol), XantPhos (25.0 mg, 43.21 μmol), Pd₂(dba)₃ (19.78 mg, 21.60 μmol), Cs₂CO₃ (140.78 mg, 432.08 μmol) and 6-(difluoromethyl)picolinamide (55.78 mg, 324.06 μmol) was purged with N₂, toluene (2.16 mL) added, the reaction vessel sealed and heated at 100° C. overnight. The reaction was cooled to rt, filtered through Celite®, concentrated in vacuo, and the crude purified by TFA modified mass directed HPLC to give N-(8-(difluoromethoxy)-2-(tetrahydro-2H-pyran-4-yl)imidazo[1,2-a]pyridin-6-yl)-6-(difluoromethyl)picolinamide 2,2,2-trifluorocetate. (44 mg, 46.46% yield). LCMS (ESI) m/z 439.2 (M+H)⁺; ¹H NMR (500 MHz, DMSO-d₆) δ ppm 1.72-1.76 (m, 2H), 1.97 (br d, J=2.44 Hz, 2H), 3.06 (br s, 1H), 3.49 (td, J=11.60, 1.83 Hz, 2H), 3.95-3.98 (m, 3H), 6.98-7.21 (m, 1H), 7.44-7.75 (m, 2H), 7.87 (br s, 1H), 8.02-8.07 (m, 1H), 8.19 (br s, 1H), 8.31-8.35 (m, 3H), 9.42 (br s, 1H), 10.90 (br s, 1H).

### Example 308: N-(8-chloro-2-(tetrahydro-2H-pyran-4-yl)imidazo[1,2-a]pyridin-6-yl)-6-(difluoromethyl)picolinamide

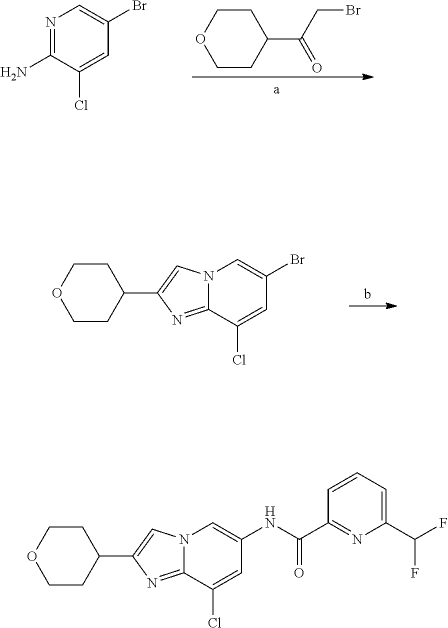 [SMILES: Nc1ncc(Br)cc1Cl ; O=C(CBr)C1CCOCC1 ; Clc1cc(Br)cn2cc(C3CCOCC3)nc12 ; O=C(Nc1cc(Cl)c2nc(C3CCOCC3)cn2c1)c1cccc(C(F)F)n1]

Step a: A mixture of 5-bromo-3-chloropyridin-2-amine (600 mg, 2.89 mmol), NaHCO₃ (728.90 mg, 8.67 mmol) and 2-bromo-1-(tetrahydro-2H-pyran-4-yl)ethan-1-one (718.61 mg, 3.47 mmol) in MeCN (5.78 mL) and toluene (2.48 mL) was stirred at 100° C. overnight. The cooled mixture was diluted with MeOH, silica gel added, the mixture concentrated in vacuo, and the powder purified (0-100% 3:1 EtOAc:EtOH in hept) to give 6-bromo-8-chloro-2-(tetrahydro-2H-pyran-4-yl)imidazo[1,2-a]pyridine (560 mg, 61.40% yield). LCMS (ESI) m/z 316.9 (M+H)⁺;

Step b: N-(8-chloro-2-(tetrahydro-2H-pyran-4-yl)imidazo[1,2-a]pyridin-6-yl)-6-(difluoromethyl)picolinamide 2,2,2-trifluoroacetate was prepared from 6-bromo-8-chloro-2-(tetrahydro-2H-pyran-4-yl)imidazo[1,2-a]pyridine and 6-(difluoromethyl)picolinamide, following the same method described in Example 307 (19.40 mg, 30.10% yield). LCMS (ESI) m/z 407.2 (M+H)⁺; ¹H NMR (500 MHz, DMSO-d₆) δ ppm 1.67-1.77 (m, 3H), 1.95 (br dd, J=12.82, 1.83 Hz, 2H), 3.04 (br t, J=11.29 Hz, 1H), 3.48 (td, J=11.75, 2.14 Hz, 1H), 3.95 (dt, J=9.61, 1.91 Hz, 2H), 6.95-7.21 (m, 1H), 7.99-8.05 (m, 1H), 8.05-8.22 (m, 1H), 8.07-8.18 (m, 1H), 8.29-8.35 (m, 2H), 9.42 (br s, 1H), 10.83 (br s, 1H).

### Example 309: N-(7-methoxy-2-(1-methyl-2-oxabicyclo[2.1.1]hexan-4-yl)imidazo[1,2-a]pyridin-6-yl)-6-(trifluoromethyl)picolinamide

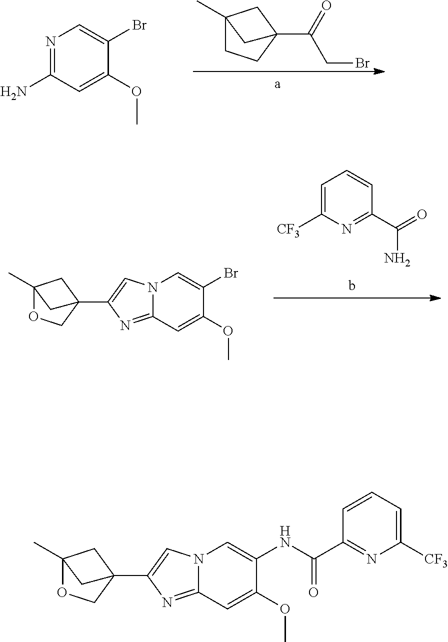

Step a: To a mixture of 5-bromo-4-methoxypyridin-2-amine (100 mg, 0.493 mmol) and 2-bromo-1-(1-methyl-2-oxabicyclo[2.1.1]hexan-4-yl)ethan-1-one (171 mg, 0.625 mmol) in tBuOH (2 mL) was added NaHCO₃ (83 mg, 0.985 mmol) and the reaction stirred at 100° C. for 16 h. The cooled mixture was concentrated in vacuo and the residue was purified by column chromatography using Combiflash® (PE/EtOAc=1/1) to give 6-bromo-7-methoxy-2-(1-methyl-2-oxabicyclo[2.1.1]hexan-4-yl)imidazo[1,2-a]pyridine (70 mg, 39.6% yield) as yellow solid. LCMS (ESI) m/z 324.8 (M+H)⁺;

Step b: To a solution of 6-bromo-7-methoxy-2-(1-methyl-2-oxabicyclo[2.1.1]hexan-4-yl)imidazo[1,2-a]pyridine (40 mg, 0.124 mmol) and 6-(trifluoromethyl)picolinamide (28 mg, 0.149 mmol) in toluene (3 mL) were added Pd₂(dba)₃ (11 mg, 0.012 mmol), XantPhos (14 mg, 0.025 mmol) and Cs₂CO₃ (80 mg, 0.248 mmol), the mixture degassed with N₂ and stirred at 110° C. for 16 h. The cooled mixture was diluted with water (30 mL) and extracted with EtOAc (30 mL×3). The combined organic extracts were washed with brine (50 mL), dried over Na₂SO₄ and filtered. The filtrate was concentrated in vacuo and the residue purified by prep-HPLC (Column: Welch Xtimate C18 150×25 mm×5 m) eluting with water (10 mm NH₄HCO₃-MeCN from 42 to 72% to give N-(7-methoxy-2-(1-methyl-2-oxabicyclo[2.1.1]hexan-4-yl)imidazo[1,2-a]pyridin-6-yl)-6-(trifluoromethyl)picolinamide (12.3 mg, 22.8% yield) as an off-white solid. LCMS (ESI) m/z 433.1 (M+H)⁺; ¹H NMR (500 MHz, METHANOL-d₄) δ ppm: 1.50 (s, 3H), 1.80-1.90 (m, 2H), 2.10-2.20 (m, 2H), 4.01 (s, 2H), 4.09 (s, 3H), 6.97 (s, 1H), 7.61 (s, 1H), 8.07 (d, J=8.0 Hz, 1H), 8.32 (t, J=8.0 Hz, 1H), 8.48 (d, J=8.0 Hz, 1H), 9.49 (s, 1H).

### Intermediate 21: 6-bromo-7-isopropoxy-2-(tetrahydro-2H-pyran-4-yl)imidazo[1,2-a]pyridine

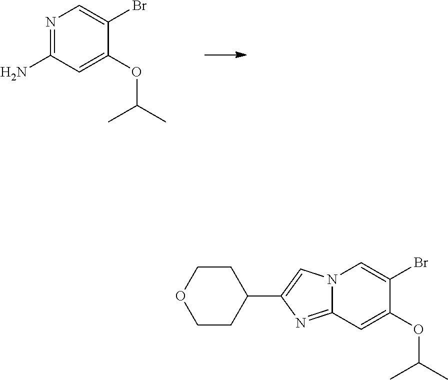 [SMILES: CC(C)Oc1cc(N)ncc1Br ; CC(C)Oc1cc2nc(C3CCOCC3)cn2cc1Br]

To a solution of 5-bromo-4-isopropoxypyridin-2-amine (100 mg, 0.433 mmol) and 2-bromo-1-(tetrahydro-2H-pyran-4-yl)ethan-1-one (99 mg, 0.476 mmol) in i-PrOH (2 mL) was added NaHCO₃ (73 mg, 0.865 mmol) and the reaction mixture stirred at 100° C. for 24 h. The cooled mixture was concentrated in vacuo and the residue was purified by silica gel column chromatography using Combiflash® (PE/EtOAc=3/1 to 1/1) to give 6-bromo-7-isopropoxy-2-(tetrahydro-2H-pyran-4-yl)imidazo[1,2-a]pyridine (80 mg, 55% yield) as yellow oil. LCMS (ESI) m/z 340.8 (M+H)⁺;

### Example 310: 6-(1,2-difluoroethyl)-N-(7-isopropoxy-2-(tetrahydro-2H-pyran-4-yl)imidazo[1,2-a]pyridin-6-yl)picolinamide

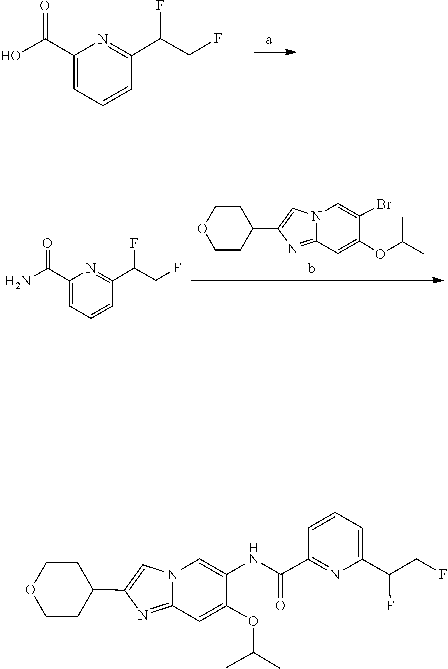

Step a: To a solution of 6-(1,2-difluoroethyl)picolinic acid prepared as in Example 239 (250 mg, 1.34 mmol) in DCM (5 mL) was added SOCl₂ (191 mg, 1.61 mmol) and DMF (1 drop) at 0° C. and the reaction stirred at 10-15° C. for 16 h. The mixture was concentrated in vacuo, the residue diluted with THF (5 mL) and NH4OH (1.68 g, 13.4 mmol, 28% purity) added. The mixture was stirred at 20° C. for 1 h then diluted with water (20 mL) and extracted with EtOAc (30 mL×3). The combined organic extracts were washed with brine (50 mL), dried over Na₂SO₄ and filtered. The filtrate was evaporated under reduced pressure to give 6-(1,2-difluoroethyl)picolinamide (150 mg, 60% yield) as an off-white solid. ¹H NMR (500 MHz, DMSO-d₆) δ ppm: 5.00-5.20 (m, 2H), 5.90-6.10 (m, 1H), 7.40-7.50 (m, 2H), 8.00-8.10 (m, 1H), 8.10-8.20 (m, 1H), 8.20 (br s, 1H).

Step b: To a solution of 6-bromo-7-isopropoxy-2-(tetrahydro-2H-pyran-4-yl)imidazo[1,2-a]pyridine (50 mg, 0.147 mmol) and 6-(1,2-difluoroethyl)picolinamide (41 mg, 0.221 mmol) in toluene (5 mL) were added Pd₂(dba)₃ (14 mg, 0.015 mmol), XantPhos (17 mg, 0.029 mmol) and Cs₂CO₃ (96 mg, 0.295 mmol), the mixture degassed with N₂ and stirred at 110° C. for 6 h. The cooled mixture was diluted with water (30 mL) and extracted with EtOAc (30 mL×3). The combined organic extracts were washed with brine (50 mL), dried over Na₂SO₄ and filtered. The filtrate was concentrated in vacuo and the residue, purified by HCl modified prep-HPLC to give 6-(1,2-difluoroethyl)-N-(7-isopropoxy-2-(tetrahydro-2H-pyran-4-yl)imidazo[1,2-a]pyridin-6-yl)picolinamide hydrochloride (12.5 mg, 18% yield) as an off-white solid. LCMS (ESI) m/z 445.1 (M+H)⁺; ¹H NMR (500 MHz, METHANOL-d₄) δ ppm: 1.60 (d, J=6.0 Hz, 6H), 1.80-1.90 (m, 2H), 2.00-2.10 (m, 2H), 3.10-3.20 (m, 1H), 3.60-3.70 (m, 2H), 4.00-4.10 (m, 2H), 5.00-5.20 (m, 3H), 5.80-6.00 (m, 1H), 7.28 (s, 1H), 7.80-7.90 (m, 2H), 8.10-8.20 (m, 1H), 8.30-8.40 (m, 1H), 9.81 (s, 1H).

### Example 311: N-(2-(3-cyanobicyclo[1.1.1]pentan-1-yl)-7-isopropoxyimidazo[1,2-a]pyridin-6-yl)-2-methoxybenzamide

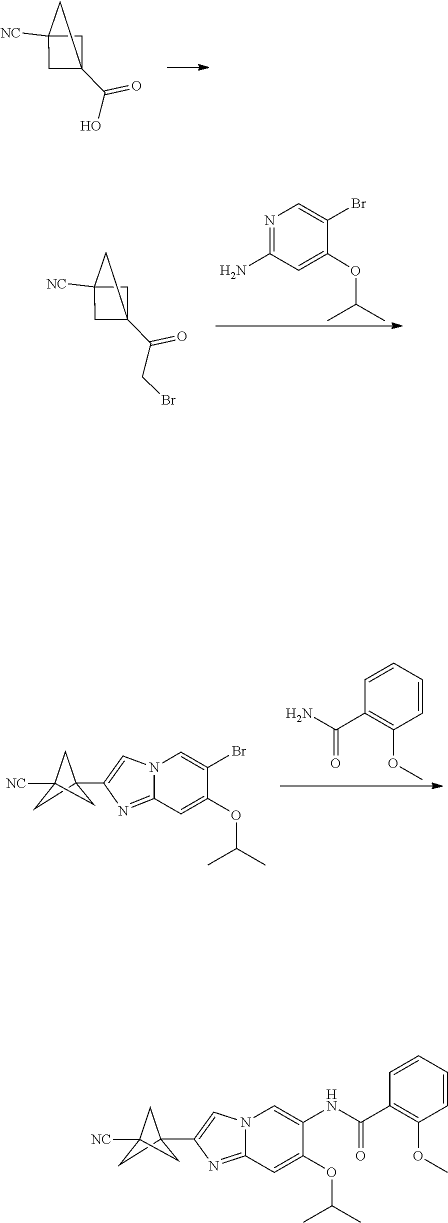 [SMILES: N#CC12CC(C(=O)O)(C1)C2 ; N#CC12CC(C(=O)CBr)(C1)C2 ; CC(C)Oc1cc(N)ncc1Br ; CC(C)Oc1cc2nc(C34CC(C#N)(C3)C4)cn2cc1Br ; COc1ccccc1C(N)=O ; COc1ccccc1C(=O)Nc1cn2cc(C34CC(C#N)(C3)C4)nc2cc1OC(C)C]

Step a: To a solution of 3-cyanobicyclo[1.1.1]pentane-1-carboxylic acid (200 mg, 1.46 mmol) in DCM (10 mL) was added (COCl)₂ (370.64 mg, 2.92 mmol) and the reaction stirred at 25° C. for 2 h. The mixture was concentrated in vacuo, the residue dissolved in THF (5 mL) and MeCN (5 mL) and the solution cooled to 0° C. TMSCHN₂ (1.1 mL, 2.19 mmol, 2 M in hexane) was added and the reaction stirred at 0° C. for 1 h. HBr (738.30 mg, 4.38 mmol, 48% in water) was added and the mixture stirred at 0° C. for 30 mins. The mixture was basified with aqueous NaHCO₃ to pH>7, diluted with EtOAc (20 mL) and washed with water (10 mL×2). The organic layer was dried over Na₂SO₄, filtered and evaporated under reduced pressure to give 3-(2-bromoacetyl)bicyclo[1.1.1]pentane-1-carbonitrile (250 mg, 71% yield) as yellow liquid. ¹H NMR (500 MHz, CDCl₃) δ: 2.61 (s, 6H), 3.88-4.13 (m, 2H),

Step b: To a mixture of 3-(2-bromoacetyl)bicyclo[1.1.1]pentane-1-carbonitrile (250 mg, 1.17 mmol) and 5-bromo-4-isopropoxypyridine-2-amine (269.89 mg, 1.17 mmol) in t-BuOH (3 mL) was added NaHCO₃ (294.35 mg, 3.50 mmol) and the reaction stirred at 80° C. for 12 h. The cooled mixture was concentrated in vacuo and the residue was purified by silica gel chromatography (PE/EtOAc from 5/1 to 1/1) to give 3-(6-bromo-7-isopropoxyimidazo[1,2-a]pyridin-2-yl)bicyclo[1.1.1]pentane-1-carbonitrile (300 mg, 66% yield) as a yellow liquid. ¹H NMR (500 MHz, CDCl₃) δ: 1.43 (d, J=6.0 Hz, 6H), 2.60 (s, 6H), 4.53-4.62 (m, 1H), 6.85 (s, 1H), 7.17 (s, 1H), 8.16 (s, 1H).

Step c: To a mixture of 3-(6-bromo-7-isopropoxyimidazo[1,2-a]pyridin-2-yl)bicyclo[1.1.1]pentane-1-carbonitrile (80 mg, 0.23 mmol) and 2-methoxybenzamide (41.91 mg, 0.28 mmol) in toluene (4 mL) were added Pd₂(dba)₃ (21.16 mg, 0.02 mmol), XantPhos (26.74 mg, 0.05 mmol) and Cs₂CO₃ (150.57 mg, 0.46 mmol) and the reaction stirred at 110° C. for 12 h. The cooled mixture was filtered through Celite® and the filtrate was evaporated under vacuum. The residue was purified by prep-HPLC (Phenomenex Synergi C18 150×30 mm×4 um), eluting with water (0.05% HCl)-MeCN from 25% to 45% to give N-(2-(3-cyanobicyclo[1.1.1]pentan-1-yl)-7-isopropoxyimidazo[1,2-a]pyridin-6-yl)-2-methoxybenzamide hydrochloride (23 mg, 23% yield) as a white solid. LCMS (ESI) m/z 417.1 (M+H)⁺ H NMR (500 MHz, METHANOL-d₄) 8: 1.62 (d, J=6.5 Hz, 6H), 2.75 (s, 6H), 4.18 (s, 3H), 5.08-5.16 (m, 1H), 7.18-7.21 (m, 1H), 7.27 (s, 1H), 7.30-7.32 (m, 1H), 7.63-7.66 (m, 1H), 7.94 (s, 1H), 8.20-8.23 (m, 1H), 9.91 (s, 1H), 10.67 (s, 1H)

### Example 312: 6-(difluoromethyl)-N-(2-(tetrahydro-2H-pyran-4-yl)-7-(trifluoromethyl)imidazo[1,2-a]pyridin-6-yl)picolinamide

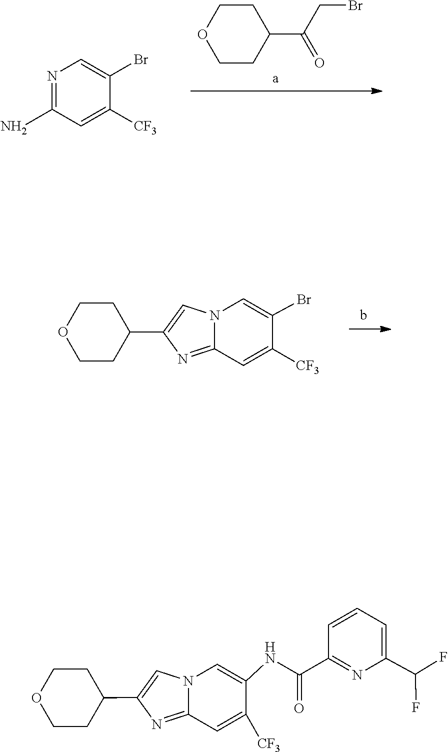 [SMILES: Nc1cc(C(F)(F)F)c(Br)cn1 ; O=C(CBr)C1CCOCC1 ; FC(F)(F)c1cc2nc(C3CCOCC3)cn2cc1Br ; O=C(Nc1cn2cc(C3CCOCC3)nc2cc1C(F)(F)F)c1cccc(C(F)F)n1]

Step a: A mixture of 5-bromo-4-(trifluoromethyl)pyridin-2-amine (400.07 mg, 1.66 mmol), 2-bromo-1-(tetrahydro-2H-pyran-4-yl)ethan-1-one (343.65 mg, 1.66 mmol) and NaHCO₃ (418.29 mg, 4.98 mmol) in EtOH (6.64 mL) was heated at 80° C. for 6 h. Further 2-bromo-1-(tetrahydro-2H-pyran-4-yl)ethan-1-one (343.65 mg, 1.66 mmol) was added and the reaction heated overnight. The reaction mixture was cooled to rt, silica gel added and the mixture concentrated in vacuo. The solid was purified by silica gel column chromatography via dry load (0-30% 3:1 EtOAc/EtOH in Hept) to give 6-bromo-2-tetrahydropyran-4-yl-7-(trifluoromethyl)imidazo[1,2-a]pyridine (250 mg, 43.13% yield). LCMS (ESI) m/z 351.0 (M+H)⁺;

Step b: 6-(difluoromethyl)-N-(2-(tetrahydro-2H-pyran-4-yl)-7-(trifluoromethyl)imidazo[1,2-a]pyridin-6-yl)picolinamide was prepared from 6-bromo-2-tetrahydropyran-4-yl-7-(trifluoromethyl)imidazo[1,2-a]pyridine and 6-(difluoromethyl)picolinamide, following the same method described in Example 307, step b. (35.80 mg, 37.85% yield). LCMS (ESI) m/z 441.2 (M+H)⁺; 1H NMR (500 MHz, DMSO-d₆) δ ppm 1.71-1.80 (m, 2H), 1.96 (br dd, J=13.12, 2.14 Hz, 2H), 3.08 (tt, J=11.37, 3.89 Hz, 1H), 3.50 (td, J=11.60, 1.83 Hz, 1H), 3.95 (dt, J=9.16, 2.14 Hz, 2H), 6.98-7.22 (m, 1H), 8.03-8.06 (m, 1H), 8.09 (s, 1H), 8.15 (s, 1H), 8.29-8.36 (m, 2H), 9.18 (s, 1H), 10.38 (s, 1H)

### Example 313: 6-(difluoromethyl)-N-(7-(1-methoxyethyl)-2-(tetrahydro-2H-pyran-4-yl)imidazo[1,2-a]pyridin-6-yl)picolinamide

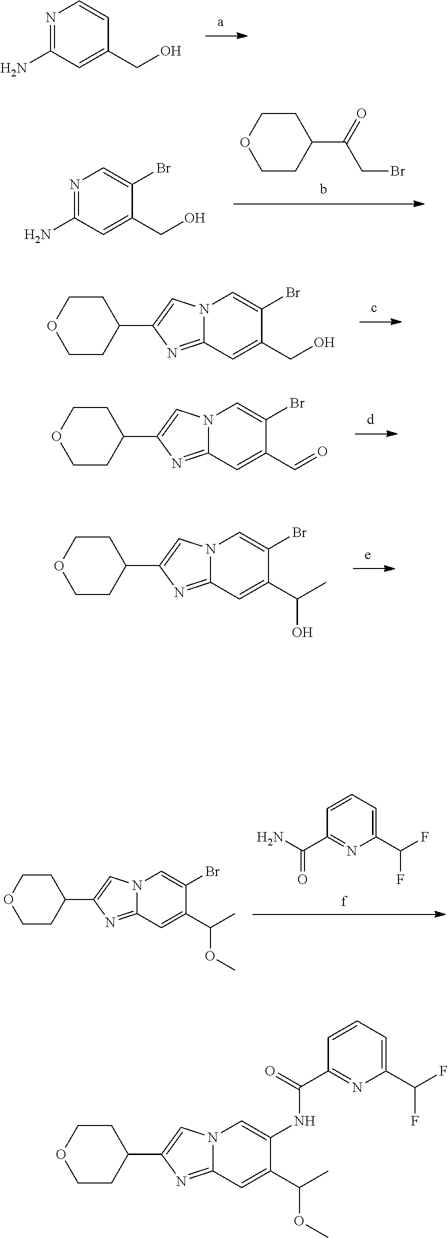

Step a: To a solution of 2-aminopyridine-4-methanol (1 g, 8.06 mmol) in MeCN (10 mL) was added NBS (1.43 g, 8.06 mmol) at 0° C. and the reaction stirred at 0° C. for 1 h. The mixture was filtered and the filter cake washed with MeCN (5 mL). The solid was dried under vacuum to give 2-amino-5-bromopyridin-4-yl)methanol (800 mg, 48% yield) as a white solid. ¹H NMR (500 MHz, DMSO-d₆) δ: 4.35 (d, J=5.5 Hz, 2H), 5.45 (t, J=5.5 Hz, 1H), 6.11 (s, 2H), 6.66 (s, 1H), 7.86 (s, 1H).

Step b: To a mixture of 2-amino-5-bromopyridin-4-yl)methanol (200 mg, 0.99 mmol) and NaHCO₃ (249.26 mg, 2.96 mmol) in EtOH (10 mL) was added 2-bromo-1-(tetrahydro-2H-pyran-4-yl)ethan-1-one (203.96 mg, 0.99 mmol) and the reaction stirred at 80° C. for 4 h. The cooled reaction was concentrated in vacuo and the residue purified by prep-TLC (DCM/MeOH=15/1) to give (6-bromo-2-(tetrahydro-2H-pyran-4-yl)imidazo[1,2-a]pyridin-7-yl)methanol (140 mg, 45% yield) as a white solid. ¹H NMR (500 MHz, DMSO-d₆) δ ppm: 1.64-1.68 (m, 2H), 1.89-1.93 (m, 2H), 2.89-2.93 (m, 1H), 3.42-3.48 (m, 2H), 3.90-3.93 (m, 2H), 4.50 (d, J=5.5 Hz, 2H), 5.56 (t, J=5.5 Hz, 1H), 7.49 (s, 1H), 7.64 (s, 1H), 8.81 (s, 1H).

Step c: To a mixture of (6-bromo-2-(tetrahydro-2H-pyran-4-yl)imidazo[1,2-a]pyridin-7-yl)methanol (100 mg, 0.32 mmol) in DCM (10 mL) was added MnO₂ (419.1 mg, 4.82 mmol) and the reaction stirred at 40° C. for 12 h. The mixture was filtered through Celite® and washed with DCM (10 mL). The filtrate was evaporated under reduced pressure to give 6-bromo-2-(tetrahydro-2H-pyran-4-yl)imidazo[1,2-a]pyridine-7-carbaldehyde (90 mg, 90% yield) as white solid. ¹H NMR (500 MHz, DMSO-d₆) δ ppm: 1.73-1.67 (m, 2H), 1.91-1.94 (m, 2H), 3.00-3.05 (m, 1H), 3.44-3.50 (m, 2H), 3.91-3.94 (m, 2H), 7.93 (s, 1H), 8.08 (s, 1H), 8.97 (s, 1H), 10.09 (s, 1H).

Step d: To a solution of 6-bromo-2-(tetrahydro-2H-pyran-4-yl)imidazo[1,2-a]pyridine-7-carbaldehyde (100 mg, 0.32 mmol) in THF (10 mL) was added MeMgBr (0.54 mL, 1.62 mmol) at 0° C. and the mixture was stirred at 15° C. for 2 h. The mixture was quenched with water (1 mL) and evaporated under vacuum. The residue was purified by prep-TLC (DCM/MeOH=10/1) to give 1-(6-bromo-2-(tetrahydro-2H-pyran-4-yl)imidazo[1,2-a]pyridin-7-yl)ethan-1-ol (50 mg, 47% yield) as a colorless liquid. LCMS (ESI) m/z 327.0 (M+H)⁺;

Step e: To a solution of 1-(6-bromo-2-(tetrahydro-2H-pyran-4-yl)imidazo[1,2-a]pyridin-7-yl)ethan-1-ol (50 mg, 0.15 mmol) in THF (5 mL) was added NaH (12.30 mg, 0.31 mmol) at 0° C. and the reaction mixture stirred at 15° C. for 30 mins. Mel (490 mg, 3.45 mmol) was added at 0° C. and the reaction stirred at 15° C. for 2 h. The mixture was quenched with water (10 mL) and extracted with EtOAc (10 mL×2). The combined organic extracts were washed with brine (5 mL), filtered and evaporated under reduced pressure. The residue was purified by prep-TLC (EtOAc) to give 6-bromo-7-(1-methoxyethyl)-2-(tetrahydro-2H-pyran-4-yl)imidazo[1,2-a]pyridine (30 mg, 51% yield) as colorless liquid. LCMS (ESI) m/z 341.0 (M+H)⁺;

Step f: To a mixture of 6-bromo-7-(1-methoxyethyl)-2-(tetrahydro-2H-pyran-4-yl)imidazo[1,2-a]pyridine (30 mg, 0.088 mmol) and 6-(difluoromethyl)picolinamide (18.27 mg, 0.11 mmol) in toluene (4 mL) was added Pd₂(dba)₃ (8.10 mg, 0.01 mmol), XantPhos (10.23 mg, 0.02 mmol) and Cs₂CO₃ (57.63 mg, 0.18 mmol). The mixture was stirred at 100° C. for 12 hours. Solvent was removed under vacuum. The residue was purified by Prep-HPLC (Phenomenex Synergi C18 150×30 mm×4 μm, water (0.05% HCl)-MeOH from 27% to 47%) to give 6-(difluoromethyl)-N-(7-(1-methoxyethyl)-2-(tetrahydro-2H-pyran-4-yl)imidazo[1,2-a]pyridin-6-yl)picolinamide (4 mg, 10% yield) as colorless liquid. LCMS (ESI) m/z 431.0 (M+H)⁺; ¹H NMR (500 MHz, METHANOL-d₄) δ ppm: 1.62 (d, J=6.5 Hz, 3H), 1.86-1.92 (m, 2H), 2.04-2.07 (m, 2H), 3.23-3.31 (m, 1H), 3.53 (s, 3H), 3.62 (t, J=12.0 Hz, 2H), 4.06-4.10 (m, 2H), 4.90-4.93 (m, 1H), 6.73-6.96 (m, 1H), 7.82 (s, 1H), 7.99-7.96 (m, 1H), 8.12 (s, 1H), 8.25-8.29 (m, 1H), 8.40-8.42 (m, 1H), 9.92 (s, 1H).

### Example 314: 6-(difluoromethyl)-N-(7-(methoxymethyl)-2-(tetrahydro-2H-pyran-4-yl)imidazo[1,2-a]pyridin-6-yl)picolinamide

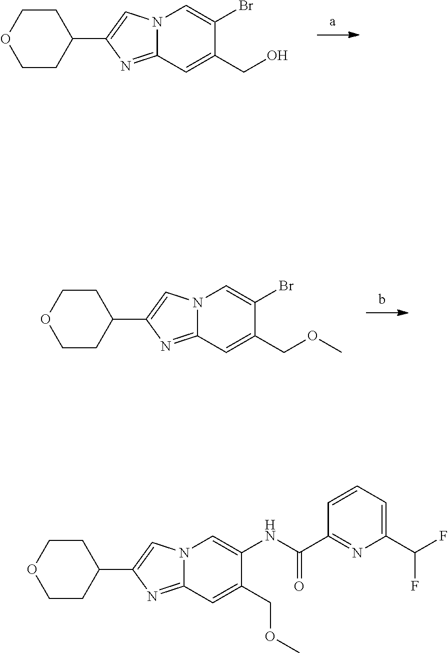 [SMILES: OCc1cc2nc(C3CCOCC3)cn2cc1Br ; COCc1cc2nc(C3CCOCC3)cn2cc1Br ; COCc1cc2nc(C3CCOCC3)cn2cc1NC(=O)c1cccc(C(F)F)n1]

Step a: NaH (38.56 mg, 964.11 μmol, 60% purity) was added to a solution of Example 313, step b: (6-bromo-2-(tetrahydro-2H-pyran-4-yl)imidazo[1,2-a]pyridin-7-yl)methanol (200 mg, 642.74 μmol) in THF (6.43 mL) at 0° C., the solution stirred for 15 min, then iodomethane (127.72 mg, 899.84 μmol) was added and the reaction stirred at rt for 2 h. The mixture was quenched with water, extracted with EtOAc (3×), dried over MgSO₄, filtered and concentrated in vacuo. The crude product was purified by silica gel column chromatography (0-50% 3:1 EtOAc/EtOH in Hept), to give 6-bromo-7-(methoxymethyl)-2-tetrahydropyran-4-yl-imidazo[1,2-a]pyridine (120 mg, 57.41% yield). LCMS (ESI) m/z 327.0 (M+H)⁺;

Step b: 6-(difluoromethyl)-N-(7-(methoxymethyl)-2-(tetrahydro-2H-pyran-4-yl)imidazo[1,2-a]pyridin-6-yl)picolinamide 2,2,2-trifluoroacetate was prepared from 6-bromo-7-(methoxymethyl)-2-tetrahydropyran-4-yl-imidazo[1,2-a]pyridine and 6-(difluoromethyl)picolinamide, following the method described in Example 315 (37.70 mg, 90.53 μmol, 45.29% yield). LCMS (ESI) m/z 417.2 (M+H)⁺; ¹H NMR (500 MHz, DMSO-d₆) δ ppm 1.69-1.77 (m, 2H), 1.92-2.00 (m, 2H), 3.17 (br t, J=11.29 Hz, 1H), 3.46-3.53 (m, 1H), 3.57 (s, 1H), 3.54-3.60 (m, 1H), 3.98 (br dd, J=11.29, 2.14 Hz, 2H), 4.74-4.87 (m, 1H), 4.81 (s, 1H), 7.01-7.26 (m, 1H), 7.92 (s, 1H), 8.06 (d, J=7.32 Hz, 1H), 8.29 (br s, 1H), 8.33-8.40 (m, 2H), 9.69 (s, 1H), 11.08 (s, 1H).

### Example 315: N-(7-(difluoromethyl)-2-(tetrahydro-2H-pyran-4-yl)imidazo[1,2-a]pyridin-6-yl)-6-methylpicolinamide

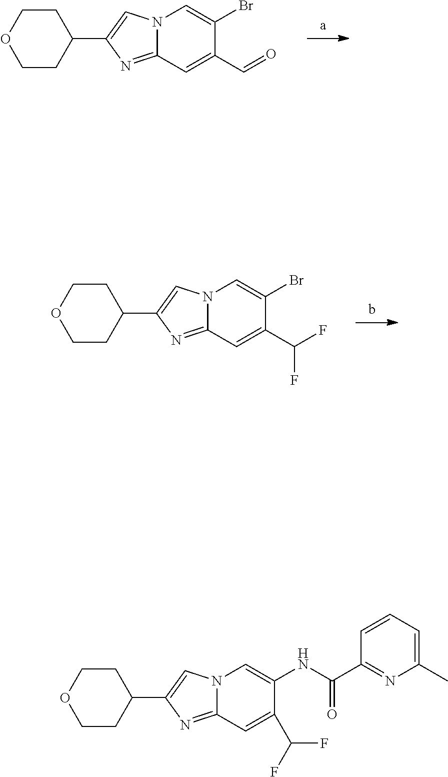 [SMILES: O=Cc1cc2nc(C3CCOCC3)cn2cc1Br ; FC(F)c1cc2nc(C3CCOCC3)cn2cc1Br ; Cc1cccc(C(=O)Nc2cn3cc(C4CCOCC4)nc3cc2C(F)F)n1]

Step a: To a mixture of Example 313, step c: 6-bromo-2-(tetrahydro-2H-pyran-4-yl)imidazo[1,2-a]pyridine-7-carbaldehyde (248 mg, 802.17 μmol) in DCM (10.03 mL) at 0° C., was added 2-methoxy-N-(2-methoxyethyl)-N-(trifluoro-λ4-sulfanyl)ethanamine (887.36 mg, 2.01 mmol, 50% purity) and the reaction allowed to warm to rt and stirred overnight. The reaction was quenched with aq. sat. NaHCO₃, extracted with DCM (3×), dried over MgSO₄, filtered and concentrated in vacuo. The residue was purified by silica gel column chromatography (0-40% EtOAc in Hept) to give 6-bromo-7-(difluoromethyl)-2-(tetrahydro-2H-pyran-4-yl)imidazo[1,2-a]pyridine (100 mg, 301.97 μmol, 37.64% yield. LCMS (ESI) m/z 333.0 (M+H)⁺;

Step b: N-(7-(difluoromethyl)-2-(tetrahydro-2H-pyran-4-yl)imidazo[1,2-a]pyridin-6-yl)-6-methylpicolinamide 2,2,2-trifluoroacetate was prepared from 6-bromo-7-(difluoromethyl)-2-(tetrahydro-2H-pyran-4-yl)imidazo[1,2-a]pyridine and 6-methylpicolinamide following the same method described in Example 306, step b (52 mg, 89.14% yield). LCMS (ESI) m/z 387.3 (M+H)⁺; ¹H NMR (500 MHz, DMSO-d₆) δ ppm 1.74 (qd, J=12.11, 3.97 Hz, 2H), 1.96 (br d, J=12.82 Hz, 2H), 2.63 (s, 3H), 3.14 (br t, J=11.60 Hz, 1H), 3.47-3.53 (m, 1H), 3.92-3.98 (m, 2H), 7.25-7.49 (m, 1H), 7.60-7.65 (m, 1H), 7.98-8.07 (m, 2H), 8.22 (br s, 1H), 9.48 (br s, 1H), 10.72 (br s, 1H).

### Example 316: N-(2-(1-cyanoethyl)-7-ethoxyimidazo[1,2-a]pyridin-6-yl)-6-(difluoromethyl)picolinamide

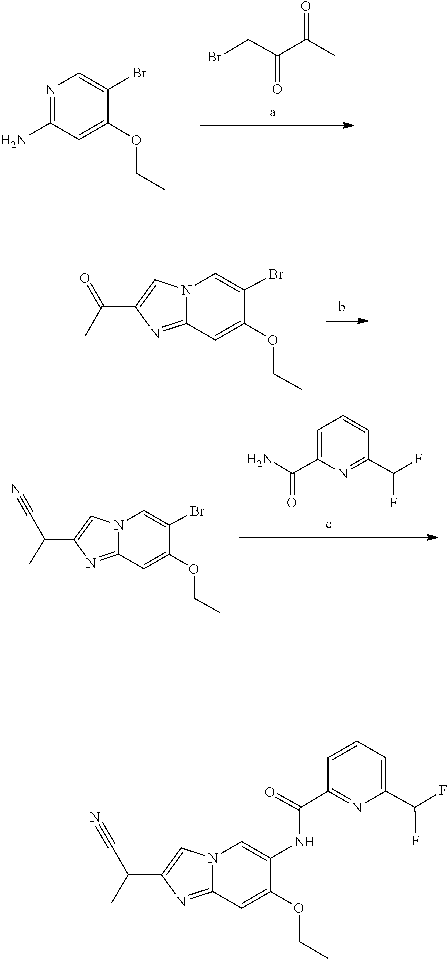

Step a: To a solution of 5-bromo-4-ethoxypyridin-2-amine (1.00 g, 4.61 mmol) in EtOH (10 mL) was added NaHCO₃ (774.07 mg, 9.22 mmol) and 1-bromobutane-2,3-dione (760.11 mg, 4.61 mmol) and the reaction stirred at 80° C. for 14 h. The cooled reaction was filtered through Celite® and the filtrate concentrated in vacuo. The residue was purified by column chromatography on silica gel using combi-flash (DCM/MeOH=20/1) and further purified by prep-TLC (DCM/MeOH=20/1) to give 1-(6-bromo-7-ethoxyimidazo[1,2-a]pyridin-2-yl)ethan-1-one (200 mg, 15% yield) as a yellow solid. ¹H NMR (400 MHz, CHLOROFORM-d) δ ppm: 1.54 (t, J=6.8 Hz, 3H), 2.68 (s, 3H), 4.18-4.12 (m, 2H), 6.92 (s, 1H), 7.94 (s, 1H), 8.28 (s, 1H).

Step b: To a mixture of TosMIC (77.58 mg, 0.4 mmol) in DMSO (5 mL) and MeOH (0.5 mL) was added KOt-Bu (148.63 mg, 1.32 mmol) and the mixture stirred at 15° C. for 15 mins. 1-(6-Bromo-7-ethoxyimidazo[1,2-a]pyridin-2-yl)ethan-1-one (75 mg, 0.26 mmol) was added and the reaction stirred at 15° C. for 45 mins. The mixture was diluted with water (20 mL) and extracted with EtOAc (10 mL×2). The combined organic extracts were washed with brine (10 mL), dried over Na₂SO₄, filtered and evaporated under reduced pressure. The residue was purified by prep-TLC (DCM/MeOH=20/1) to give 2-(6-bromo-7-ethoxyimidazo[1,2-a]pyridin-2-yl)propanenitrile (60 mg, 73% yield) as white solid. ¹H NMR (300 MHz, CDCl₃): 1.53 (t, J=6.8 Hz, 3H), 1.75 (d, J=7.2 Hz, 3H), 4.04-4.16 (m, 3H), 6.86 (s, 1H), 7.43 (s, 1H), 8.21 (s, 1H).

Step c: To a mixture of 2-(6-bromo-7-ethoxyimidazo[1,2-a]pyridin-2-yl)propanenitrile (30 mg, 0.1 mmol) and 6-(difluoromethyl)picolinamide (21.07 mg, 0.12 mmol) in toluene (4 mL) was added Pd₂(dba)₃ (9.34 mg, 0.01 mmol), XantPhos (11.8 mg, 0.02 mmol) and Cs₂CO₃ (66.46 mg, 0.2 mmol) and the reaction stirred at 100° C. for 12 h. The cooled mixture was evaporated under reduced pressure and the residue purified by acid mediated prep-HPLC to give N-(2-(1-cyanoethyl)-7-ethoxyimidazo[1,2-a]pyridin-6-yl)-6-(difluoromethyl)picolinamide (10 mg, 25% yield) as white solid. LCMS (ESI) m/z 386.0 (M+H)⁺; ¹H NMR (500 MHz, METHANOL-d₄) δ ppm: 1.68 (t, J=7.0 Hz, 3H), 1.81 (d, J=7.0 Hz, 3H), 4.46-4.51 (m, 2H), 4.57-4.62 (m, 1H), 6.96-6.99 (m, 1H), 7.33 (s, 1H), 7.96-7.99 (m, 1H), 8.15 (s, 1H), 8.26-8.30 (m, 1H), 8.39-8.42 (m, 1H), 9.82 (s, 1H), 10.77 (s, 1H).

### Intermediate 22: 6-bromo-2-(1-methyl-2-oxabicyclo[2.1.1]hexan-4-yl)-7-((1,1,1-trifluoropropan-2-yl)oxy)imidazo[1,2-a]pyridine

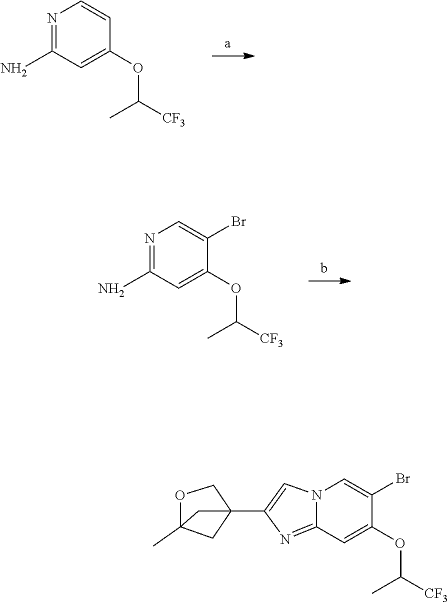 [SMILES: CC(Oc1ccnc(N)c1)C(F)(F)F ; CC(Oc1cc(N)ncc1Br)C(F)(F)F ; CC(Oc1cc2nc(C34COC(C)(C3)C4)cn2cc1Br)C(F)(F)F]

Step a: 5-bromo-4-(2,2,2-trifluoropropan-2-yl)oxy)pyridin-2-amine was prepared from 4-((1,1,1-trifluoropropan-2-yl)oxy)pyridin-2-amine, following the same method described in Example 306, step a, (850 mg, 87.70% yield. LCMS (ESI) m/z 284.9 (M+H)⁺;

Step b: A mixture of NaHCO₃ (442.06 mg, 5.26 mmol), 5-bromo-4-(2,2,2-trifluoropropan-2-yl)oxy)pyridin-2-amine (500 mg, 1.75 mmol) and 2-bromo-1-(1-methyl-2-oxabicyclo[2.1.1]hexan-4-yl)ethan-1-one (384.27 mg, 1.75 mmol) in MeCN (2.92 mL) and toluene (2.92 mL) was stirred at 90° C. overnight. The reaction mixture was cooled to rt, MeOH and silica gel added and the mixture concentrated in vacuo. The solid was purified by silica gel column chromatography (0-50% 3:1 EtOAc:EtOH in heptane) to obtain 6-bromo-2-(1-methyl-2-oxabicyclo[2.1.1]hexan-4-yl)-7-((1,1,1-trifluoropropan-2-yl)oxy)imidazo[1,2-a]pyridine (500 mg, 70.51% yield). LCMS (ESI) m/z 405.1, 407.1 (M+H)⁺;

### Example 317, 318 and 319: 2-methoxy-N-(2-(1-methyl-2-oxabicyclo[2.1.1]hexan-4-yl)-7-((1,1,1-trifluoropropan-2-yl)oxy)imidazo[1,2-a]pyridin-6-yl)nicotinamide and 2-methoxy-N-(2-(1-methyl-2-oxabicyclo[2.1.1]hexan-4-yl)-7-((1,1,1-trifluoropropan-2-yl)oxy)imidazo[1,2-a]pyridin-6-yl)nicotinamide, Enantiomers 1 and 2

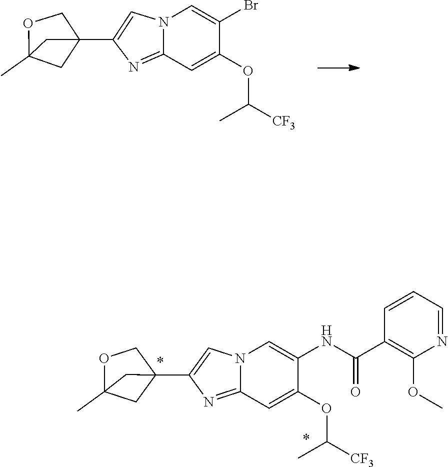 [SMILES: CC(Oc1cc2nc(C34COC(C)(C3)C4)cn2cc1Br)C(F)(F)F ; COc1ncccc1C(=O)Nc1cn2cc(C34COC(C)(C3)C4)nc2cc1OC(C)C(F)(F)F]

Example 317, 2-methoxy-N-(2-(1-methyl-2-oxabicyclo[2.1.1]hexan-4-yl)-7-((1,1,1-trifluoropropan-2-yl)oxy)imidazo[1,2-a]pyridin-6-yl)nicotinamide 2,2,2-trifluoroacetate was prepared from Intermediate 22: 6-bromo-2-(1-methyl-2-oxabicyclo[2.1.1]hexan-4-yl)-7-((1,1,1-trifluoropropan-2-yl)oxy)imidazo[1,2-a]pyridine and 2-methoxypyridine-3-carboxamide, following the method described in Example 307, step b (47.20 mg, 64.78% yield). LCMS (ESI) m/z 477.3 (M+H)⁺; ¹H NMR (500 MHz, DMSO-d₆) δ ppm 1.42-1.52 (m, 3H), 1.64 (d, J=6.71 Hz, 3H), 1.86 (br d, J=3.66 Hz, 2H), 2.13 (br s, 2H), 3.85-4.01 (m, 2H), 4.07-4.19 (m, 3H), 5.91 (br s, 1H), 7.32 (dd, J=7.94, 4.88 Hz, 1H), 7.62 (br s, 1H), 8.08-8.29 (m, 1H), 8.45-8.54 (m, 1H), 9.85 (br s, 1H), 10.31 (br s, 1H)

The compound was further purified by SFC using a LUX Cellulose-4 LC 30×250 mm, 5 μm column eluting with 40% MeOH w/0.1% DEA in CO₂ (flow rate: 100 mL/min, ABPR 120 bar, MBPR 40 psi, column temp 40° C.) to give enantiomer 1.

¹H NMR (400 MHz, CDCl₃) δ ppm 1.58 (s, 2H), 1.67-1.79 (m, 1H), 1.72 (br d, J=4.52 Hz, 2H), 2.01 (br s, 2H), 2.17 (br d, J=12.80 Hz, 2H), 4.10 (s, 2H), 4.24 (s, 3H), 4.98 (br s, 1H), 7.18 (dd, J=7.53, 4.77 Hz, 1H), 7.36 (s, 1H), 8.41 (br d, J=3.26 Hz, 1H), 8.61 (dd, J=7.53, 2.01 Hz, 1H), 9.75 (br d, J=8.03 Hz, 1H), 10.36 (br s, 1H).

Further Elution Gave Enantiomer 2.

¹H NMR (400 MHz, CDCl₃) δ ppm 1.57 (s, 3H) 1.72 (d, J=6.53 Hz, 3H) 1.97-2.04 (m, 2H) 2.25 (br s, 2H) 4.10 (s, 2H) 4.24 (s, 3H) 5.01 (br s, 1H) 7.18 (dd, J=7.53, 4.77 Hz, 1H) 7.35 (s, 1H) 8.41 (br d, J=3.51 Hz, 1H) 8.61 (dd, J=7.78, 2.01 Hz, 1H) 9.78 (br s, 1H) 10.39 (br s, 1H).

### Example 320: N-(7-cyclopropoxy-2-(tetrahydro-2H-pyran-4-yl)imidazo[1,2-a]pyridin-6-yl)-6-(difluoromethyl)picolinamide

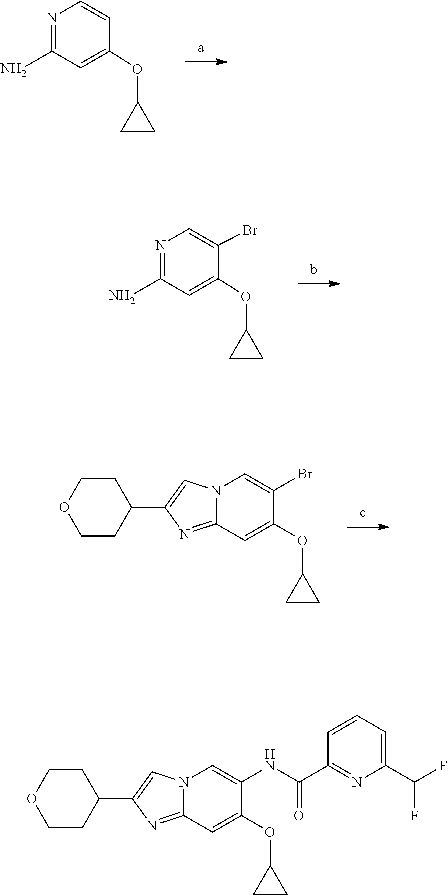 [SMILES: Nc1cc(OC2CC2)ccn1 ; Nc1cc(OC2CC2)c(Br)cn1 ; Brc1cn2cc(C3CCOCC3)nc2cc1OC1CC1 ; O=C(Nc1cn2cc(C3CCOCC3)nc2cc1OC1CC1)c1cccc(C(F)F)n1]

Step a: 1-Bromopyrrolidine-2,5-dione (711.92 mg, 4.00 mmol) was added to a mixture of 4-(cyclopropoxy)pyridin-2-amine (600.72 mg, 4.00 mmol) in MeCN (8 mL) at 0° C. and the reaction stirred at rt for 2 h. The reaction was quenched with aq. sat. NaHCO₃, extracted with EtOAc (3×), the combined organic layers washed with brine, dried over MgSO₄, filtered and evaporated under reduced pressure to give 5-bromo-4-(cyclopropoxy)pyridin-2-amine (900 mg, 3.93 mmol, 98.22% yield). LCMS (ESI) m/z 229.0 (M+H)⁺;

Step b: A mixture of 5-bromo-4-(cyclopropoxy)pyridin-2-amine (700.95 mg, 3.06 mmol), 2-bromo-1-(tetrahydro-2H-pyran-4-yl)ethan-1-one (633.60 mg, 3.06 mmol) and NaHCO₃ (771.21 mg, 9.18 mmol, 357.04 uL) in MeCN (4.37 mL) and toluene (4.37 mL) was heated at 90° C. overnight. The reaction mixture cooled to rt, silica gel added and the mixture concentrated, in vacuo. The solid was purified by column chromatography on silica gel via dry load (0-30% 3:1 EtOAc/EtOH in Hept) to obtain 6-bromo-7-(cyclopropoxy)-2-tetrahydropyran-4-yl-imidazo[1,2-a]pyridine (650 mg, 1.93 mmol, 62.99% yield) LCMS (ESI) m/z 337.0 (M+H)⁺;

Step c: N-(7-cyclopropoxy-2-(tetrahydro-2H-pyran-4-yl)imidazo[1,2-a]pyridin-6-yl)-6-(difluoromethyl)picolinamide 2,2,2-trifluoroacetate was prepared from 6-bromo-7-(cyclopropoxy)-2-tetrahydropyran-4-yl-imidazo[1,2-a]pyridine and 6-(difluoromethyl)picolinamide following the method described in Example 307 step a, (42.80 mg, 99.90 μmol, 48.12% yield). LCMS (ESI) m/z 429.3 (M+H)⁺; ¹H NMR (500 MHz, DMSO-d₆) δ ppm 0.93-0.99 (m, 2H) 1.01-1.05 (m, 2H) 1.64-1.80 (m, 2H) 1.96 (br dd, J=12.51, 2.14 Hz, 2H) 3.13 (br t, J=11.90 Hz, 1H) 3.47-3.51 (m, 2H) 3.95-3.99 (m, 2H) 4.41 (br d, J=3.05 Hz, 1H) 7.05-7.30 (m, 1H) 7.53 (s, 1H) 8.06 (dd, J=6.71, 2.44 Hz, 1H) 8.15 (s, 1H) 8.34-8.38 (m, 1H) 9.72 (s, 1H) 10.41 (s, 1H)

### Example 321: N-(7-chloro-8-methyl-2-(tetrahydro-2H-pyran-4-yl)imidazo[1,2-a]pyridin-6-yl)-3-(difluoromethyl)benzamide

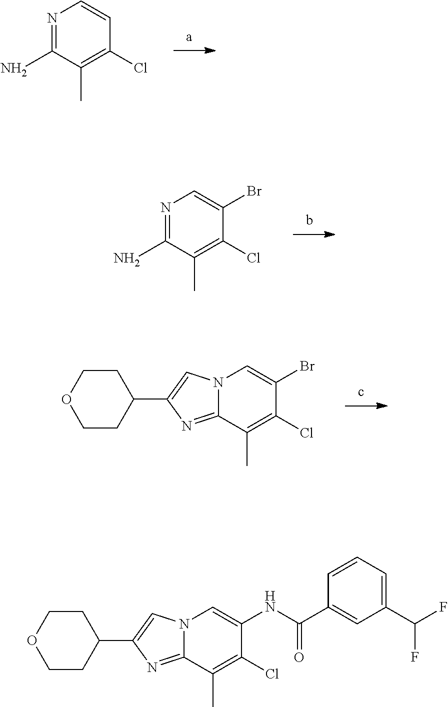

6-bromo-7-chloro-8-methyl-2-(tetrahydro-2H-pyran-4-yl)imidazo[1,2-a]pyridine was prepared from 4-chloro-3-methylpyridin-2-amine and 2-bromo-1-(tetrahydro-2H-pyran-4-yl)ethan-1-one, following the same synthetic sequence described in Example 320 steps a and b (560 mg, 1.77 mmol, 61.40% yield). LCMS (ESI) m/z 316.9 (M+H)⁺;

Step c: A vial charged with 6-bromo-7-chloro-8-methyl-2-(tetrahydro-2H-pyran-4-yl)imidazo[1,2-a]pyridine (110 mg, 333.72 μmol), XantPhos (32.83 mg, 56.73 μmol), Pd₂(dba)₃ (24.45 mg, 26.70 μmol), Cs₂CO₃ (217.46 mg, 667.44 μmol) and 6-(difluoromethyl)picolinamide (54.57 mg, 317.03 μmol) was purged with N₂ and closed with a screw cap with septa. Toluene (2.43 mL) was added at rt and the vial sealed and heated at 100° C. overnight. The reaction mixture was cooled to rt, filtered, concentrated in vacuo and the residue purified by TFA modified mass directed HPLC to give N-(7-chloro-8-methyl-2-(tetrahydro-2H-pyran-4-yl)imidazo[1,2-a]pyridin-6-yl)-3-(difluoromethyl)benzamide 2,2,2-trifluoroacetate (80.10 mg, 190.33 μmol, 57.03% yield). LCMS (ESI) m/z 421.3 (M+H); ¹H NMR (400 MHz, CDCl₃) δ ppm 1.83 (qd, J=12.34, 4.14 Hz, 2H), 2.11 (br d, J=10.79 Hz, 2H), 2.70-2.87 (m, 3H), 3.21-3.36 (m, 1H), 3.56-3.68 (m, 2H), 4.13 (dd, J=11.67, 3.39 Hz, 2H), 6.59-6.90 (m, 1H), 7.49 (s, 1H), 7.95 (d, J=7.78 Hz, 1H), 8.18 (t, J=7.78 Hz, 1H), 8.42 (d, J=7.78 Hz, 1H), 9.81 (s, 1H), 10.79 (s, 1H).

### Example 322: 6-(difluoromethyl)-N-(8-fluoro-7-isopropoxy-2-(1-methyl-2-oxabicyclo[2.1.1]hexan-4-yl)imidazo[1,2-a]pyridin-6-yl)picolinamide

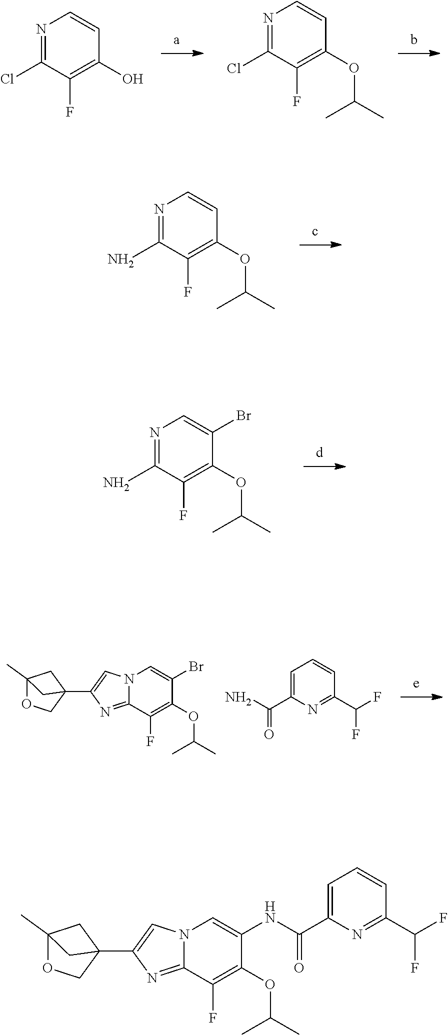

Step a: 2-Iodopropane (11.52 g, 67.78 mmol) was added to a solution of 2-chloro-3-fluoropyridin-4-ol (5.00 g, 33.89 mmol) and K₂CO₃ (14.05 g, 101.67 mmol) in acetone (169.45 mL) and the reaction heated at 50° C. for 2 days. The cooled mixture was filtered, concentrated in vacuo and the residue purified by column chromatography to give 2-chloro-3-fluoro-4-isopropoxypyridine (5.10 g, 26.90 mmol, 79.37% yield). LCMS (ESI) m/z 189.9 (M+H)⁺;

Step b: A mixture of 2-chloro-3-fluoro-4-isopropoxypyridine (5.10 g, 26.90 mmol), t-butyl carbamate (4.41 g, 37.66 mmol), XantPhos-Pd-G3 (1.28 g, 1.35 mmol) and Cs₂CO₃ (17.53 g, 53.80 mmol) in toluene (100 mL) was degassed and the reaction stirred at 70° C. overnight. The cooled mixture was evaporated under reduced pressure. The residue was suspended in dioxane (65 mL), 4M HCl (13 mL) added and the reaction stirred at rt until all starting material had been consumed. The solution was concentrated in vacuo and the crude purified by silica gel column chromatography to give 3-fluoro-4-isopropoxypyridin-2-amine hydrochloride, 1.30 g, 29.4%. LCMS (ESI) m/z 171.0 (M+H)⁺.

Step c: A mixture of 3-fluoro-4-isopropoxypyridin-2-amine hydrochloride (1.30 g, 7.64 mmol) and NBS (1.36 g, 7.64 mmol) in MeCN (76.4 mL) was stirred at rt for 10 mins. The reaction was diluted with sat. aq. NaHCO₃, extracted with EtOAc, the combined organic extracts dried over Na₂SO₄, filtered and concentrated in vacuo. The crude product was purified by silica gel column chromatography using a gradient of 0-10% EtOH in EtOAc to give 5-bromo-3-fluoro-4-isopropoxypyridin-2-amine (1.32 g, 5.30 mmol). LCMS (ESI) m/z 248.9 (M+H)⁺

Step d: A mixture of 5-bromo-3-fluoro-4-isopropoxypyridin-2-amine (1.20 g, 4.82 mmol), 2-bromo-1-(1-methyl-2-oxabicyclo[2.1.1]hexan-4-yl)ethan-1-one (1.06 g, 4.82 mmol) and NaHCO₃ (1.21 g, 14.46 mmol) in MeCN (28.92 mL) was heated at 80° C. overnight. The reaction was cooled to rt, filtered and concentrated in vacuo. The crude was purified by silica gel column chromatography using (0-100% EtOAc in Heptane) to give 6-bromo-8-fluoro-7-isopropoxy-2-(1-methyl-2-oxabicyclo[2.1.1]hexan-4-yl)imidazo[1,2-a]pyridine (750 mg, 42.12% yield). LCMS (ESI) m/z 370.9 (M+H)+

Step e: A mixture of 6-bromo-8-fluoro-7-isopropoxy-2-(1-methyl-2-oxabicyclo[2.1.1]hexan-4-yl)imidazo[1,2-a]pyridine (51 mg, 138.13 μmol), 6-(difluoromethyl)picolinamide (33.29 mg, 193.38 μmol), XantPhos-Pd-G3 (13.10 mg, 13.81 μmol) and Cs₂CO₃ (90.01 mg, 276.3 μmol) in toluene (1.35 mL) under N₂ was stirred overnight at 100° C. The cooled reaction was partitioned between water and EtOAc and the organic phase concentrated in vacuo. The crude was purified by silica gel column chromatography (0-100% EtOAc-Heptane) to give 6-(difluoromethyl)-N-(8-fluoro-7-isopropoxy-2-(1-methyl-2-oxabicyclo[2.1.1]hexan-4-yl)imidazo[1,2-a]pyridin-6-yl)picolinamide. LCMS (ESI) m/z 461.0 (M+H)⁺. ¹H NMR (500 MHz, DMSO-d₆) δ ppm 1.37-1.46 (m, 9H), 1.77 (dd, J=1.37, 4.27 Hz, 2H), 2.01 (dd, J=1.37, 4.27 Hz, 2H), 3.89 (s, 2H), 4.72 (td, J=5.78, 12.40 Hz, 1H), 7.01-7.30 (m, 1H), 7.98-8.11 (m, 2H), 8.25-8.40 (m, 2H), 9.38 (s, 1H), 10.43 (s, 1H),

### Example 323: 2-(difluoromethoxy)-N-(8-fluoro-7-isopropoxy-2-(1-methyl-2-oxabicyclo[2.1.1]hexan-4-yl)imidazo[1,2-a]pyridin-6-yl)nicotinamide

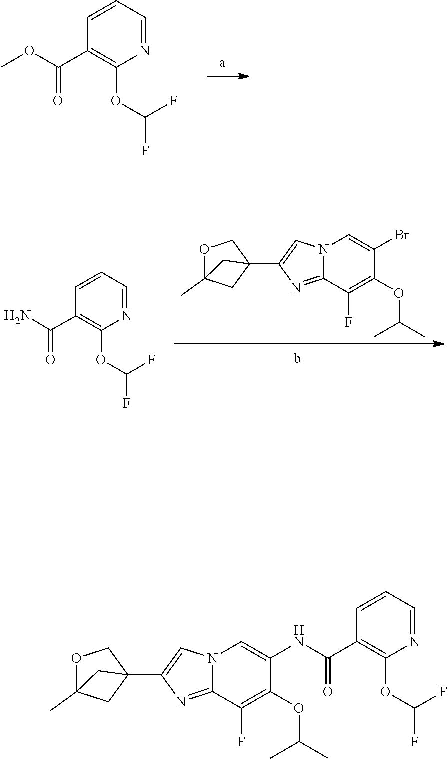 [SMILES: COC(=O)c1cccnc1OC(F)F ; NC(=O)c1cccnc1OC(F)F ; CC(C)Oc1c(Br)cn2cc(C34COC(C)(C3)C4)nc2c1F ; CC(C)Oc1c(NC(=O)c2cccnc2OC(F)F)cn2cc(C34COC(C)(C3)C4)nc2c1F]

Step a: To a solution of methyl 2-(difluoromethoxy)nicotinate (PCT Int. Appl. 2018, 2018065962) (260 mg, 1.28 mmol) in THF (2 mL) was added NH₄OH (3 mL) and the reaction stirred at 40° C. for 2 h. The mixture was neutralized using 1M HCl aq, diluted with water (50 mL) and then extracted with EtOAc (100 mL×3). The combined organic extracts were washed with brine (50 mL), dried over Na₂SO₄, and filtered. The filtrate was concentrated in vacuo and the residue purified by column chromatography on silica gel using Combiflash® (PE/EtOAc=10/1 to 3/1) to give 2-(difluoromethoxy)nicotinamide (200 mg, 74.75% yield) as white solid. LCMS (ESI) m/z 188.0 (M+H)⁺. ¹H NMR (500 MHz, CDCl₃) δ ppm: 6.02-5.85 (m, 1H), 7.29 (dd, J=5.0 Hz, 8.0 Hz, 1H), 7.50-7.79 (m, 1H), 8.32 (dd, J=2.0 Hz, 5.0 Hz, 1H), 8.62 (dd, J=2.0 Hz, 8.0 Hz, 1H)

Step b: To a solution of Example 322 step d: 6-bromo-8-fluoro-7-isopropoxy-2-(1-methyl-2-oxabicyclo[2.1.1]hexan-4-yl)imidazo[1,2-a]pyridine (30 mg, 81.25 μmol) in toluene (2 mL) was added 2-(difluoromethoxy)nicotinamide (30.57 mg, 162.50 μmol), XantPhos (9.40 mg, 16.25 μmol), Cs₂CO₃ (52.95 mg, 162.50 μmol) and Pd₂(dba)₃ (7.44 mg, 8.12 μmol) under N₂ and the reaction stirred at 100° C. for 16 h. The cooled reaction mixture was filtered and the filtrate was purified by prep-HPLC (Column: Welch Xtimate C18 150×25 mm×5 m) eluting with water (10 mm NH₄HCO₃-MeCN from 42 to 72% to give 2-(difluoromethoxy)-N-(8-fluoro-7-isopropoxy-2-(1-methyl-2-oxabicyclo[2.1.1]hexan-4-yl)imidazo[1,2-a]pyridin-6-yl)nicotinamide as an off-white solid.

LCMS (ESI) m/z 477.2 (M+H)⁺; ¹H NMR (500 MHz, METHANOL-d₄) δ ppm: 1.48 (d, J=6.0 Hz, 6H), 1.52 (s, 3H), 1.90 (dd, J=1.5 Hz, 4.5 Hz, 2H), 2.15 (dd, J=1.5 Hz, 4.5 Hz, 2H), 4.04 (s, 2H), 4.96-5.02 (m, 1H), 7.45 (dd, J=5.0 Hz, 7.5 Hz, 1H), 7.75-8.09 (m, 2H), 8.45 (dd, J=2.0 Hz, 5.0 Hz, 1H), 8.60 (dd, J=2.0 Hz, 7.5 Hz, 1H), 9.45 (s, 1H).

### Example 324: 1-(difluoromethyl)-N-(8-fluoro-7-isopropoxy-2-(1-methyl-2-oxabicyclo[2.1.1]hexan-4-yl)imidazo[1,2-a]pyridin-6-yl)-2-oxo-1,2-dihydropyridine-3-carboxamide

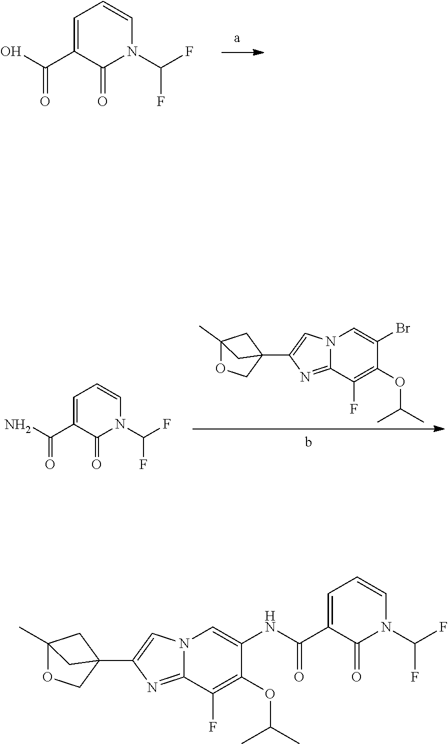 [SMILES: O=C(O)c1cccn(C(F)F)c1=O ; NC(=O)c1cccn(C(F)F)c1=O ; CC(C)Oc1c(Br)cn2cc(C34COC(C)(C3)C4)nc2c1F ; CC(C)Oc1c(NC(=O)c2cccn(C(F)F)c2=O)cn2cc(C34COC(C)(C3)C4)nc2c1F]

Step a: (COCl)₂ (368.60 mg, 2.90 mmol) was added to a solution of 1-(difluoromethyl)-2-oxo-pyridine-3-carboxylic acid (499.28 mg, 2.64 mmol) in DCM (4.40 mL) and the reaction stirred overnight. DIPEA (682.39 mg, 5.28 mmol), followed by ammonia (0.4 M, 13.20 mL) were added and the solution was stirred at rt. The mixture was concentrated in vacuo, the residue was re-dissolved in brine and extracted with EtOAc. The combined organic extracts were washed with brine, dried and evaporated under reduced pressure to give 1-(difluoromethyl)-2-oxo-pyridine-3-carboxamide, which was used without further purification. ¹H NMR (400 MHz, METHANOL-d₄) δ ppm: 6.68 (t, J=7.03 Hz, 1H) 7.65-7.94 (m, 1H) 8.03 (dd, J=7.28, 2.26 Hz, 1H) 8.46-8.60 (m, 1H)

Step b: A mixture of Example 322 step d: 6-bromo-8-fluoro-7-isopropoxy-2-(1-methyl-2-oxabicyclo[2.1.1]hexan-4-yl)imidazo[1,2-a]pyridine (90 mg, 243.75 μmol), XPhos Pd G3 (10.41 mg, 12.19 μmol), K₃PO₄ (103.48 mg, 487.50 μmol), and 1-(difluoromethyl)-2-oxo-pyridine-3-carboxamide (91.71 mg, 487.50 μmol) was purged with N₂ and closed with a screw cap with septa. tBuOH (1.22 mL) was added, the vial sealed and heated at 80° C. overnight. The reaction mixture was cooled to rt, filtered, diluted with water and extracted with EtOAc. The combined organic extracts were dried, concentrated in vacuo and the residue purified by TFA mediated reverse phase HPLC to obtain 1-(difluoromethyl)-N-(8-fluoro-7-isopropoxy-2-(1-methyl-2-oxabicyclo[2.1.1]hexan-4-yl)imidazo[1,2-a]pyridin-6-yl)-2-oxo-1,2-dihydropyridine-3-carboxamide 2,2,2-trifluoroacetate (2.40 mg, 4.07 μmol, 1.67% yield). LCMS (ESI) m/z 476.9 (M+H)⁺; ¹H NMR (500 MHz, METHANOL-d₄) δ ppm: 1.54 (s, 3H), 1.59 (d, J=5.49 Hz, 5H), 1.94-2.05 (m, 3H), 2.10-2.28 (m, 2H), 4.06 (s, 2H), 5.26 (td, J=6.10, 1.83 Hz, 1H), 6.80 (t, J=7.02 Hz, 1H), 7.77-8.21 (m, 3H), 8.72 (dd, J=7.33, 1.83 Hz, 1H), 9.78 (s, 1H), 12.35 (s, 1H).

### Example 325: N-(8-fluoro-7-isopropoxy-2-(1-methyl-2-oxabicyclo[2.2.1]heptan-4-yl)imidazo[1,2-a]pyridin-6-yl)-1-methyl-1H-pyrazole-3-carboxamide

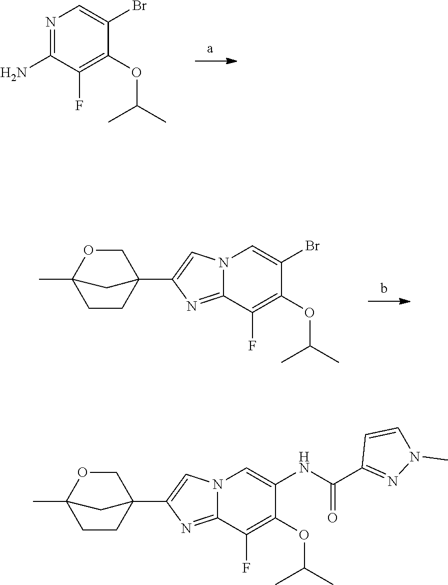

Step a: A mixture of Example 322, step c: 5-bromo-3-fluoro-4-isopropoxypyridin-2-amine (775.90 mg, 3.09 mmol), 2-bromo-1-(1-methyl-2-oxabicyclo[2.2.1]heptan-4-yl)ethan-1-one (720.28 mg, 3.09 mmol) and K₂CO₃ (854.14 mg, 6.18 mmol) in MeCN (30.90 mL) was heated at 80° C. overnight. The cooled reaction mixture was concentrated in vacuo and the residue purified by silica gel column chromatography using a gradient of 0-100% EtOAc-heptane to give 6-bromo-8-fluoro-7-isopropoxy-2-(1-methyl-2-oxabicyclo[2.2.1]heptan-4-yl)imidazo[1,2-a]pyridine. LCMS (ESI) m/z 383.0 (M+H)⁺;

Step b: A mixture of 6-bromo-8-fluoro-7-isopropoxy-2-(1-methyl-2-oxabicyclo[2.2.1]heptan-4-yl)imidazo[1,2-a]pyridine (123 mg, 320.94 μmol), 1-methylpyrazole-3-carboxamide (48.19 mg, 385.13 μmol), XantPhos-Pd-G3 (30.44 mg, 32.09 μmol) and Cs₂CO₃ (418.27 mg, 1.28 mmol) in toluene (3.21 mL) was degassed with N₂ and the reaction stirred at 80° C. overnight. The cooled mixture was evaporated under reduced pressure and the residue purified by reverse phase HPLC (acid gradient) of 5% to 70% MeCN in water. LCMS (ESI) m/z 428.0 (M+H)⁺; ¹H NMR (500 MHz, METHANOL-d₄) δ ppm: 1.48 (d, J=4.27 Hz, 6H), 1.50 (s, 3H), 1.79-1.94 (m, 2H), 1.97-2.04 (m, 2H), 2.07-2.14 (m, 1H), 2.14-2.22 (m, 1H), 3.94-3.98 (m, 1H), 4.03 (s, 3H), 4.04-4.08 (m, 1H), 4.80-4.86 (m, 1H), 6.85 (d, J=2.29 Hz, 1H), 7.74 (dd, J=2.67, 6.94 Hz, 2H), 9.25 (d, J=1.22 Hz, 1H).

### Example 326, 327 and 328: N-(7-(sec-butoxy)-8-fluoro-2-(1-methyl-2-oxabicyclo[2.1.1]hexan-4-yl)imidazo[1,2-a]pyridin-6-yl)-2-methoxynicotinamide and N-(7-(sec-butoxy)-8-fluoro-2-(1-methyl-2-oxabicyclo[2.1.1]hexan-4-yl)imidazo[1,2-a]pyridin-6-yl)-2-methoxynicotinamide Enantiomer 1 and 2

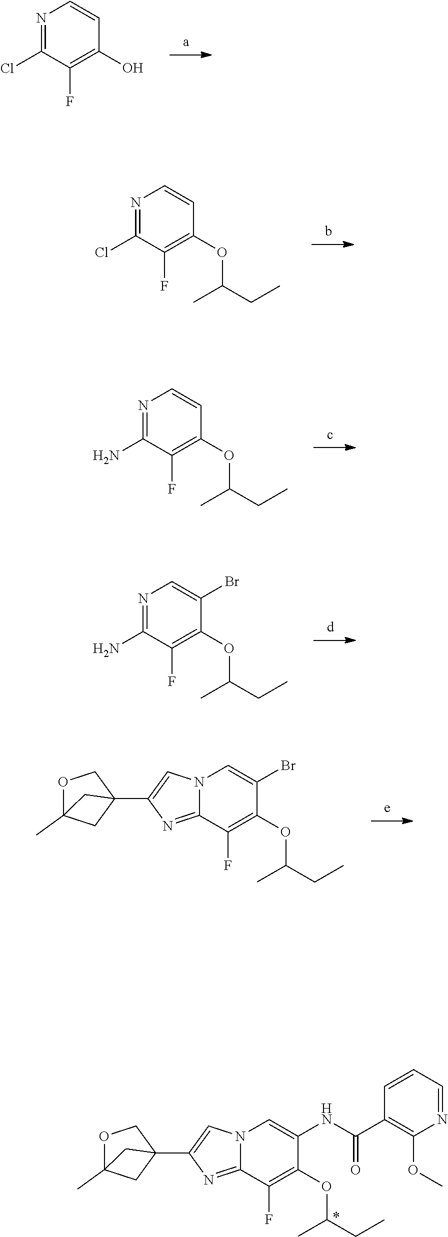

Step a: A mixture of 2-chloro-3-fluoropyridin-4-ol (1.00 g, 6.78 mmol), K₂CO₃ (2.81 g, 20.34 mmol) and 2-iodobutane (2.50 g, 13.56 mmol) in acetone (33.90 mL) was heated under reflux overnight. The cooled mixture was filtered, the solid dissolved in EtOAc and washed with water. The organic solution was dried over Na₂SO₄ and evaporated under reduced pressure to afford 4-(sec-butoxy)-2-chloro-3-fluoropyridine. LCMS (ESI) m/z 203.0 (M+H)⁺;

Step b: A mixture of 4-(sec-butoxy)-2-chloro-3-fluoropyridine (891.95 mg, 4.38 mmol), K₂CO3 (1.21 g, 8.76 mmol), BrettPhos Pd G3 (396.68 mg, 438.0 μmol) and tert-butylcarbamate (1.02 g, 8.76 mmol) in tBuOH (43.80 mL) was degassed with N₂ and the reaction stirred at 80° C. overnight. The cooled reaction was diluted with water, extracted with EtOAc and the combined organic extracts evaporated under reduced pressure. The crude product was purified by column chromatography on silica gel (0-100% EtOAc-Heptane) to give tert-butyl (4-(sec-butoxy)-3-fluoropyridin-2-yl)carbamate. This was then stirred in 4N HCl in dioxane for 2 h and evaporated under reduced pressure to give 4-(sec-butoxy)-3-fluoropyridin-2-amine. LCMS (ESI) m/z 185.0 (M+H)⁺;

Step c: NBS (211.80 mg, 1.19 mmol) was added to a solution of 4-(sec-butoxy)-3-fluoropyridin-2-amine (220 mg, 1.19 mmol) in MeCN (10 mL) and the reaction stirred for 1 h. The reaction was diluted with water and extracted with EtOAc. The combined organic extracts were evaporated under reduced pressure and the residue purified by column chromatography on silica gel (0-100% EtOAc-Heptane) to give 5-bromo-4-(sec-butoxy)-3-fluoropyridin-2-amine. LCMS (ESI) m/z 264.0 (M+H)⁺;

Step d: A mixture of 5-bromo-4-(sec-butoxy)-3-fluoropyridin-2-amine (80 mg, 304.06 μmol), 2-bromo-1-(1-methyl-2-oxabicyclo[2.1.1]hexan-4-yl)ethan-1-one (66.61 mg, 304.06 μmol) and NaHCO₃ (76.63 mg, 912.18 μmol) in MeCN (5 mL) was heated at 80° C. overnight. The reaction was cooled to rt, filtered through a frit and the filtrate concentrated in vacuo. The residue was purified by column chromatography on silica gel (0-100% EtOAc in Hept) to obtain 6-bromo-7-(sec-butoxy)-8-fluoro-2-(1-methyl-2-oxabicyclo[2.1.1]hexan-4-yl)imidazo[1,2-a]pyridine (36 mg, 93.93 μmol, 30.89% yield) LCMS (ESI) m/z 383.0 (M+H)⁺;

Step d: A mixture of 6-bromo-7-(sec-butoxy)-8-fluoro-2-(1-methyl-2-oxabicyclo[2.1.1]hexan-4-yl)imidazo[1,2-a]pyridine (36 mg, 93.93 μmol) and 2-methoxynicotinamide (17.15 mg, 112.72 μmol) XantPhos-Pd-G3 (8.91 mg, 9.39 μmol) and Cs₂CO₃ (122.42 mg, 375.72 μmol) in toluene (469.65 uL) was degassed with N₂ and heated at 80° C. overnight. The cooled reaction was evaporated under reduced pressure and the residue purified by TFA modified reverse phase HPLC to give N-(7-(sec-butoxy)-8-fluoro-2-(1-methyl-2-oxabicyclo[2.1.1]hexan-4-yl)imidazo[1,2-a]pyridin-6-yl)-2-methoxynicotinamide 2,2,2-trifluoroacetate. LCMS (ESI) m/z 455.0 (M+H)⁺;

The compound was further purified by SFC using CHIRALPAK IB 30×250 mm, 5 μm eluting with 30% MeOH w/0.1% DEA in CO₂ (flow rate: 100 mL/min, ABPR 120 bar, MBPR 40 psi, column temp 40° C.) to give enantiomer 1.

¹H NMR (500 MHz, DMSO-d₆) δ ppm: 0.98 (t, J=7.40 Hz, 3H), 1.34 (d, J=6.10 Hz, 3H), 1.43 (s, 3H), 1.65-1.74 (m, 1H), 1.76 (dd, J=1.60, 4.20 Hz, 2H), 1.78-1.87 (m, 1H), 2.00 (dd, J=1.45, 4.20 Hz, 2H), 3.88 (s, 2H), 4.17 (s, 3H), 4.56-4.63 (m, 1H), 7.29 (t, J=6.18 Hz, 1H), 7.97 (d, J=3.05 Hz, 1H), 8.42-8.49 (m, 2H), 9.42 (s, 1H), 10.27 (s, 1H).

Further elution provided the second enantiomer.

¹H NMR (500 MHz, DMSO-d₆) δ ppm: 0.98 (t, J=7.40 Hz, 3H), 1.34 (d, J=6.10 Hz, 3H), 1.43 (s, 3H), 1.66-1.74 (m, 1H), 1.76 (dd, J=1.53, 4.27 Hz, 2H), 1.78-1.88 (m, 1H), 2.00 (dd, J=1.45, 4.35 Hz, 2H), 3.88 (s, 2H), 4.17 (s, 3H), 4.56-4.65 (m, 1H), 7.29 (t, J=6.18 Hz, 1H), 7.97 (d, J=3.05 Hz, 1H), 8.40-8.50 (m, 2H), 9.42 (s, 1H), 10.27 (s, 1H).

### Example 329: 6-(2,2-difluoroethoxy)-N-(8-fluoro-7-isopropoxy-2-(1-methyl-2-oxabicyclo[2.1.1]hexan-4-yl)imidazo[1,2-a]pyridin-6-yl)picolinamide

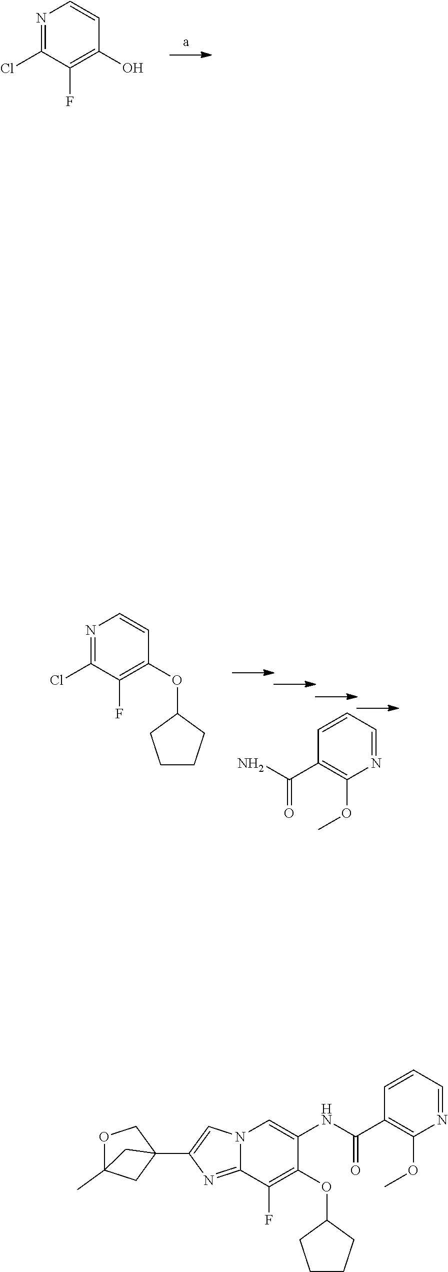 [SMILES: Oc1ccnc(Cl)c1F ; Fc1c(OC2CCCC2)ccnc1Cl ; COc1ncccc1C(N)=O ; COc1ncccc1C(=O)Nc1cn2cc(C34COC(C)(C3)C4)nc2c(F)c1OC1CCCC1]

Step a: Triphenylphosphine (on solid support, 1.77 g, 5.39 mmol, 80% purity), cyclopentanol (437.86 mg, 5.09 mmol) and DIAD (1.10 g, 5.42 mmol) were added sequentially to 2-chloro-3-fluoropyridin-4-ol (500 mg, 3.39 mmol) in THF (20 mL) and the reaction stirred for 2 h at rt.

The mixture was filtered and concentrated in vacuo. The resulting solid was purified by column chromatography on silica gel (0-65% EtOAc-Heptane) to afford 2-chloro-4-(cyclopentyloxy)-3-fluoropyridine. LCMS (ESI) m/z 215.9 (M+H)⁺;

6-(2,2-difluoroethoxy)-N-(8-fluoro-7-isopropoxy-2-(1-methyl-2-oxabicyclo[2.1.1]hexan-4-yl)imidazo[1,2-a]pyridin-6-yl)picolinamide 2,2,2-trifluoroacetate was prepared from 2-chloro-4-(cyclopentyloxy)-3-fluoropyridine and 2-methoxynicotinamide, following a similar synthetic sequence to that described in Example 326. LCMS (ESI) m/z 467.0 (M+H)⁺; ¹H NMR (500 MHz, METHANOL-d₄) δ ppm: 1.42 (s, 3H), 1.62-1.73 (m, 2H), 1.74-1.84 (m, 2H), 1.89 (dd, J=1.68, 4.58 Hz, 2H), 1.99-2.07 (m, 4H), 2.12 (dd, J=1.68, 4.58 Hz, 2H), 3.95 (s, 2H), 4.15 (s, 3H), 5.48-5.57 (m, 1H), 7.14 (dd, J=4.81, 7.55 Hz, 1H), 7.97 (d, J=2.29 Hz, 1H), 8.33 (dd, J=1.91, 4.81 Hz, 1H), 8.47 (dd, J=1.83, 7.63 Hz, 1H), 9.68 (s, 1H), 10.30 (s, 1H).

### Example 330: 6-(difluoromethyl)-N-(2-(tetrahydro-2H-pyran-4-yl)imidazo[1,2-a]pyrazin-6-yl)picolinamide

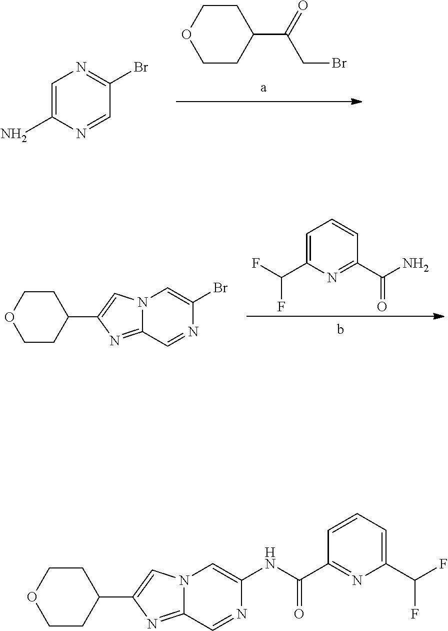

Step a: A solution of 2-bromo-1-(tetrahydro-2H-pyran-4-yl)ethan-1-one (3.20 g, 15.43 mmol) and 5-bromopyrazin-2-amine (1.79 g, 10.29 mmol) in EtOH (100 mL) was heated at 80° C. for 6 h. NaHCO₃ (1.73 g, 20.58 mmol) was added and heating continued for an additional 12 h. The mixture was cooled to rt, the inorganic solids were filtered off and the solvent evaporated under reduced pressure. The residue was purified by HPLC to give 6-bromo-2-tetrahydropyran-4-yl-imidazo[1,2-a]pyrazine (770 mg, 26.52% yield). LCMS (ESI) m/z 283.0 (M+H)⁺;

Step b: A mixture of 6-(difluoromethyl)picolinamide (91.51 mg, 531.65 μmol), 6-bromo-2-tetrahydropyran-4-yl-imidazo[1,2-a]pyrazine (125 mg, 443.04 μmol), KOtBu (69.60 mg, 620.26 μmol) and (R)-(+)-BINAP (27.59 mg, 44.30 μmol) in dioxane (50 mL) was degassed under Ar (g). Pd₂(dba)₃ (12.74 mg, 22.15 μmol) was added and the reaction heated under reflux for 2 days. The cooled mixture was concentrated in vacuo and the residue suspended in MeCN (50 mL). The suspension was filtered through a silica pad and the filtrate evaporated. The resulting brown solid was purified by HPLC to yield 6-(difluoromethyl)-N-(2-(tetrahydro-2H-pyran-4-yl)imidazo[1,2-a]pyrazin-6-yl)picolinamide (17.60 mg, 10.64% yield). LCMS (ESI) m/z 374.2 (M+H)⁺; ¹H NMR (400 MHz, CDCl₃) ppm: 1.77-1.95 (m, 2H), 1.96-2.07 (m, 2H), 3.00-3.12 (m, 1H), 3.44-3.62 (m, 2H), 4.03-4.12 (m, 2H), 6.71 (t, J=55.0 Hz, 1H), 7.54 (s, 1H), 7.86 (d, J=7.8 Hz, 1H), 8.10 (t, J=7.8 Hz, 1H), 8.39 (d, J=7.8 Hz, 1H), 8.84 (s, 1H), 9.33 (s, 1H), 10.18 (s, 1H).

### Example 331: N-(7-chloro-8-methoxy-2-(tetrahydro-2H-pyran-4-yl)imidazo[1,2-a]pyridin-6-yl)-6-(difluoromethyl)picolinamide

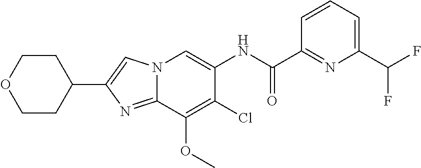 [SMILES: COc1c(Cl)c(NC(=O)c2cccc(C(F)F)n2)cn2cc(C3CCOCC3)nc12]

N-(7-chloro-8-methoxy-2-(tetrahydro-2H-pyran-4-yl)imidazo[1,2-a]pyridin-6-yl)-6-(difluoromethyl)picolinamide 2,2,2-trifluoroacetate was prepared from 4-chloro-3-methoxypyridin-2-amine, 2-bromo-1-(tetrahydro-2H-pyran-4-yl)ethan-1-one and 6-(difluoromethyl)picolinamide following a similar method to that described in Example 322. LCMS (ESI) m/z 437.2 (M+H)⁺; ¹H NMR (400 MHz, CDCl₃) δ ppm 1.86 (qd, J=12.42, 4.39 Hz, 2H), 2.14 (br dd, J=12.80, 2.01 Hz, 2H), 3.33 (tt, J=11.95, 3.61 Hz, 1H), 3.64 (td, J=11.92, 1.76 Hz, 2H), 4.15 (dd, J=11.80, 3.26 Hz, 2H), 4.23 (s, 3H), 6.61-6.92 (m, 1H), 7.54 (s, 1H), 7.97 (d, J=7.78 Hz, 1H), 8.21 (t, J=7.78 Hz, 1H), 8.44 (d, J=7.28 Hz, 1H), 9.66-9.78 (m, 1H), 10.76 (s, 1H).

### Example 332: 2-cyclopropyl-N-(2-(tetrahydro-2H-pyran-4-yl)imidazo[1,2-a]pyrazin-6-yl)thiazole-4-carboxamide

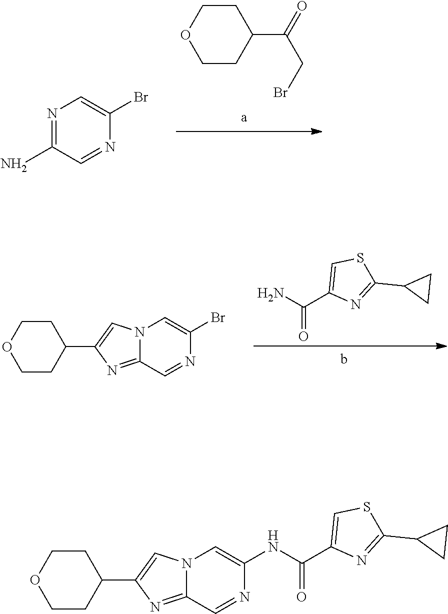 [SMILES: O=C(CBr)C1CCOCC1 ; Nc1cnc(Br)cn1 ; Brc1cn2cc(C3CCOCC3)nc2cn1 ; NC(=O)c1csc(C2CC2)n1 ; O=C(Nc1cn2cc(C3CCOCC3)nc2cn1)c1csc(C2CC2)n1]

Step a: To a solution of 2-bromo-1-(tetrahydro-2H-pyran-4-yl)ethan-1-one (400 mg, 1.93 mmol) and 5-bromopyrazin-2-amine (134.45 mg, 772.72 μmol) in tBuOH (5 mL) was added NaHCO₃ (194.75 mg, 2.32 mmol) and the reaction stirred at 100° C. for 96 h. The reaction was cooled to rt, concentrated in vacuo and the crude purified by prep-HPLC to give 6-bromo-2-(tetrahydro-2H-pyran-4-yl)imidazo[1,2-a]pyrazine (150 mg, 489.12 μmol, 63.36% yield) as a yellow oil. LCMS (ESI) m/z 281.9 (M+H)⁺;

Step b: Pd₂(dba)₃ (10.19 mg, 0.0177 mmol), XantPhos (20.51 mg, 0.0284 mmol) and Cs₂CO₃ (173.23 mg, 0.531 mmol) were added to a solution of 6-bromo-2-(tetrahydro-2H-pyran-4-yl)imidazo[1,2-a]pyrazine (30 mg, 0.177 mmol) and 2-cyclopropylthiazole-4-carboxamide (45.99 mg, 0.284 mmol) in toluene (4 mL) and the reaction purged with N₂. The reaction mixture was stirred at 110° C. for 12 h, then cooled and evaporated under reduced pressure. The residue was purified by prep-HPLC (Column: Phenomenex Synergi C18 150×30 mm×4 um; eluting with water 1 (0.05% HCl-MeCN, from 27% to 47%), to give 2-cyclopropyl-N-(2-(tetrahydro-2H-pyran-4-yl)imidazo[1,2-a]pyrazin-6-yl)thiazole-4-carboxamide hydrochloride (2.00 mg, 0.055 mmol, 3.88% yield) as a brown solid. LCMS (ESI) m/z 370.0 (M+H)⁺; ¹H NMR (500 MHz, METHANOL-d₄) δ ppm: 1.16-1.19 (m, 2H), 1.24-1.27 (m, 2H), 1.88-1.92 (m, 2H), 2.05-2.08 (m, 2H), 2.45-2.51 (m, 1H), 3.30-3.32 (m, 1H), 3.60-3.66 (m, 2H), 4.06-4.10 (m, 2H), 8.23 (s, 1H), 8.30 (s, 1H), 9.15 (d, J=0.5 Hz, 1H), 9.64 (d, J=1.0 Hz, 1H).

### Example 333: 2-(difluoromethyl)-N-(8-methoxy-2-(tetrahydro-2H-pyran-4-yl)imidazo[1,2-a]pyrazin-6-yl)oxazole-4-carboxamide

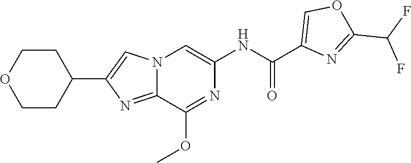 [SMILES: COc1nc(NC(=O)c2coc(C(F)F)n2)cn2cc(C3CCOCC3)nc12]

2-(Difluoromethyl)-N-(8-methoxy-2-(tetrahydro-2H-pyran-4-yl)imidazo[1,2-a]pyrazin-6-yl)oxazole-4-carboxamide 2,2,2-trifluoroacetate was prepared from 5-bromo-3-methoxypyrazin-2-amine, 2-bromo-1-(tetrahydro-2H-pyran-4-yl)ethan-1-one and 2-(difluoromethyl)oxazole-4-carboxamide, following the synthetic sequence described in Example 308 (32.70 mg, 63.81 μmol, 39.84% yield). LCMS (ESI) m/z 394.3 (M+H)⁺; ¹H NMR (500 MHz, DMSO-d₆) δ ppm 1.63-1.76 (m, 2H), 1.91 (br dd, J=13.12, 2.14 Hz, 2H), 2.97 (tt, J=11.22, 3.74 Hz, 1H), 3.49 (d, J=2.44 Hz, 1H), 3.93 (dt, J=9.46, 1.98 Hz, 2H), 4.09 (s, 3H), 6.96-7.48 (m, 1H), 8.06 (s, 1H), 8.89 (s, 1H), 9.14 (s, 1H), 9.98 (br s, 1H).

### Example 334: 6-cyclopropyl-N-(8-methoxy-2-(tetrahydro-2H-pyran-4-yl)imidazo[1,2-a]pyrazin-6-yl)picolinamide

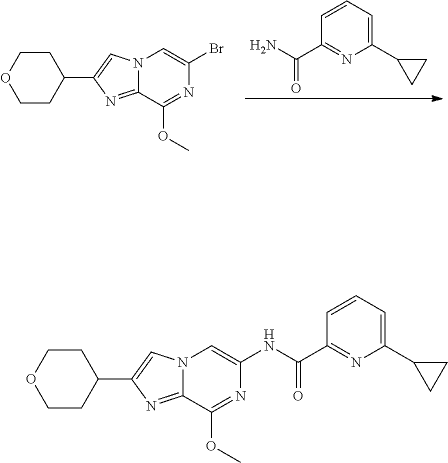 [SMILES: COc1nc(Br)cn2cc(C3CCOCC3)nc12 ; NC(=O)c1cccc(C2CC2)n1 ; COc1nc(NC(=O)c2cccc(C3CC3)n2)cn2cc(C3CCOCC3)nc12]

6-cyclopropyl-N-(8-methoxy-2-(tetrahydro-2H-pyran-4-yl)imidazo[1,2-a]pyrazin-6-yl)picolinamide hydrochloride was prepared from 5-bromo-3-methoxypyrazin-2-amine, 2-bromo-1-(tetrahydro-2H-pyran-4-yl)ethan-1-one and 6-(cyclopropyl)picolinamide, following a similar synthetic sequence to that described in Example 309 (5 mg, 7.62% yield) as a brown solid. LCMS (ESI) m/z 394.0 (M+H)⁺; 1H NMR (500 MHz, METHANOL-d₄) 8:1.24-1.15 (m, 4H), 1.85-1.94 (m, 2H), 2.03-2.07 (m, 2H), 2.31-2.37 (m, 1H), 3.30-3.31 (m, 1H), 3.59-3.64 (m, 2H), 4.06-4.10 (m, 2H), 4.34 (s, 3H), 7.57 (d, J=7.5 Hz, 1H), 8.03 (t, J=7.5 Hz, 1H), 8.10 (d, J=6.5 Hz, 1H), 8.27 (s, 1H), 9.28 (d, J=1.0 Hz, 1H).

### Example 335: 2-methoxy-N-(2-(1-methyl-2-oxabicyclo[2.1.1]hexan-4-yl)-8-propoxyimidazo[1,2-a]pyrazin-6-yl)benzamide

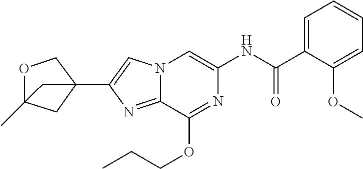 [SMILES: CCCOc1nc(NC(=O)c2ccccc2OC)cn2cc(C34COC(C)(C3)C4)nc12]

2-Methoxy-N-(2-(1-methyl-2-oxabicyclo[2.1.1]hexan-4-yl)-8-propoxyimidazo[1,2-a]pyrazin-6-yl)benzamide 2,2,2-trifluoroacetate was prepared from 3-propoxypyrazin-2-amine, 2-bromo-1-(1-methyl-2-oxabicyclo[2.1.1]hexan-4-yl)ethan-1-one and 2-methoxybenzamide, using the same synthetic sequence as described in Example 321 (28.50 mg, 53.12 μmol, 22.01% yield). LCMS (ESI) m/z 423.4 (M+H); ¹H NMR (500 MHz, DMSO-d₆) δ ppm 0.97-1.05 (m, 4H), 1.44 (s, 4H), 1.79 (dd, J=4.27, 1.22 Hz, 2H), 1.83-1.91 (m, 3H), 2.04 (dd, J=4.27, 1.22 Hz, 2H), 3.91 (s, 2H) 4.01 (s, 3H), 4.45 (t, J=6.71 Hz, 2H), 7.14 (t, J=7.63 Hz, 1H), 7.26 (d, J=7.94 Hz, 1H), 7.58 (td, J=7.94, 1.83 Hz, 1H), 7.89 (dd, J=7.63, 1.53 Hz, 1H), 8.11 (s, 1H), 8.97 (s, 1H), 10.26 (s, 1H).

### Example 336: N-(8-Isopropoxy-2-(1-methyl-2-oxabicyclo[2.1.1]hexan-4-yl)imidazo[1,2-a]pyrazin-6-yl)pyrazolo[1,5-a]pyridine-7-carboxamide

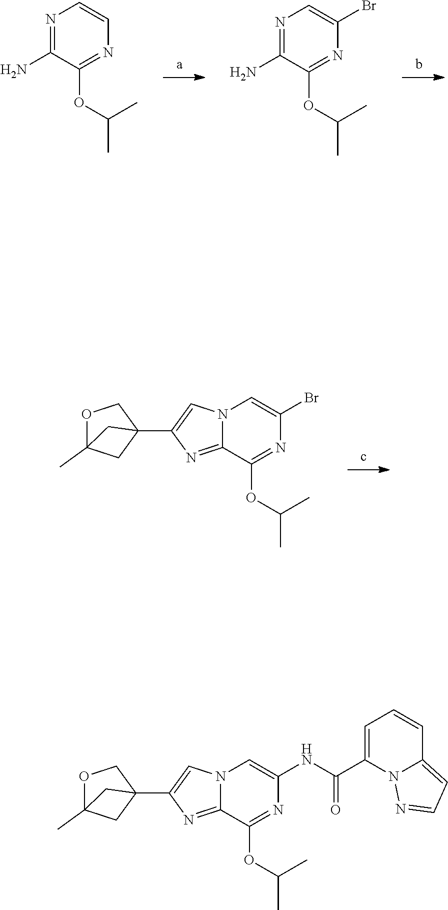 [SMILES: CC(C)Oc1nccnc1N ; CC(C)Oc1nc(Br)cnc1N ; CC(C)Oc1nc(Br)cn2cc(C34COC(C)(C3)C4)nc12 ; CC(C)Oc1nc(NC(=O)c2cccc3ccnn23)cn2cc(C34COC(C)(C3)C4)nc12]

Step a: 1-Bromopyrrolidine-2,5-dione (2.90 g, 16.32 mmol) was added to a solution of 3-isopropoxypyrazin-2-amine (2.50 g, 16.32 mmol) in MeCN (32 mL) at 0° C. and the reaction stirred at rt for 2 h. The mixture was quenched with aq. sat. NaHCO₃ soln., extracted with EtOAc (3×) and the combined organic extracts dried over MgSO₄, filtered and concentrated in vacuo. The crude was purified by silica gel column chromatography (dry load, of 3:1 EtOAc/EtOH in heptanes, 0-50%) to give 5-bromo-3-isopropoxypyrazin-2-amine (1.4 g, 40% yield). LCMS (ESI) m/z 234.1 (M+H)⁺.

Step b: A mixture of 5-bromo-3-isopropoxypyrazin-2-amine (400 mg, 1.72 mmol), 2-bromo-1-(1-methyl-2-oxabicyclo[2.1.1]hexan-4-yl)ethan-1-one (376 mg, 1.72 mmol) and NaHCO₃ (433 mg, 5.16 mmol) in MeCN (3.4 mL) and toluene (3.4 mL) was heated at 90° C. for 16 h. Silica and MeOH were added to the cooled reaction and the mixture concentrated in vacuo. The powder was purified by silica gel column chromatography (dry load, 0-30% gradient of 3:1 EtOAC/EtOH in Heptanes) to give 6-bromo-8-isopropoxy-2-(1-methyl-2-oxabicyclo[2.1.1]hexan-4-yl)imidazo[1,2-a]pyrazine (350 mg, 57% yield). LCMS (ESI) m/z 354.1 (M+H)⁺.

Step c: A vial charged with 6-bromo-8-isopropoxy-2-(1-methyl-2-oxabicyclo[2.1.1]hexan-4-yl)imidazo[1,2-a]pyrazine (60 mg, 170.3 μmol), pyrazolo[1,5-a]pyridine-7-carboxamide (41 mg, 255.5 μmol), Pd₂(dba)₃ (15.6 mg, 17 μmol), XantPhos (19.7 mg, 34 μmol), and Cs₂CO₃ (111 mg, 340 μmol) was purged with N₂ and closed with a screw cap with septa. Toluene (1.7 mL) was added via syringe and the reaction heated at 100° C. for 16 h. The reaction was cooled to rt, the mixture filtered through Celite® and the filtrate concentrated in vacuo. The residue was purified by TFA modified mass directed HPLC to provide N-(8-isopropoxy-2-(1-methyl-2-oxabicyclo[2.1.1]hexan-4-yl)imidazo[1,2-a]pyrazin-6-yl)pyrazolo[1,5-a]pyridine-7-carboxamide 2,2,2-trifluoroacetate (34.5 mg, 46% yield). LCMS (ESI) m/z 433.3 (M+H)⁺. ¹H NMR (500 MHz, DMSO-d₆) δ ppm 1.40-1.50 (m, 9H) 1.79 (dd, J=4.27, 1.83 Hz, 2H) 2.04 (dd, J=4.27, 1.83 Hz, 2H) 3.91 (s, 2H) 5.55 (spt, J=6.21 Hz, 1H) 6.98 (d, J=2.44 Hz, 1H) 7.51 (dd, J=8.85, 7.02 Hz, 1H) 7.89-8.02 (m, 1H) 8.07-8.20 (m, 2H) 8.35 (d, J=2.44 Hz, 1H) 9.08 (s, 1H) 12.93 (s, 1H).

### Example 337: N-(8-isopropoxy-2-(1-methyl-2-oxabicyclo[2.1.1]hexan-4-yl)imidazo[1,2-a]pyrazin-6-yl)-2-methoxynicotinamide

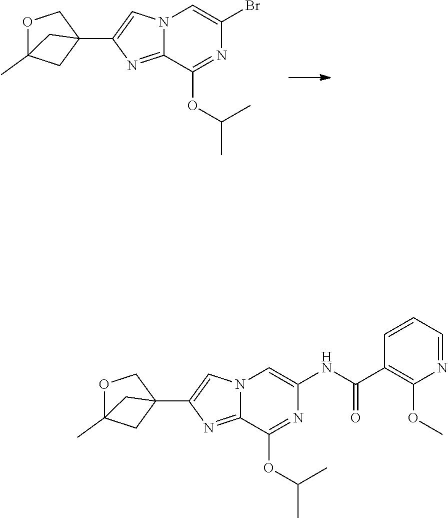 [SMILES: CC(C)Oc1nc(Br)cn2cc(C34COC(C)(C3)C4)nc12 ; COc1ncccc1C(=O)Nc1cn2cc(C34COC(C)(C3)C4)nc2c(OC(C)C)n1]

A vial charged with Example 336, step b: 6-bromo-8-isopropoxy-2-(1-methyl-2-oxabicyclo[2.1.1]hexan-4-yl)imidazo[1,2-a]pyrazine (60 mg, 170.3 μmol), 2-methoxynicotinamide (39 mg, 255.5 μmol), Pd₂(dba)₃ (15.6 mg, 17 μmol), XantPhos (19.7 mg, 34 μmol) and Cs₂CO₃ (111 mg, 340 μmol) was purged with N₂ and closed with a screw cap with septa. Toluene (1.7 mL) was added via syringe and the mixture was heated at 100° C. for 16 h. After cooling to room temperature, the mixture was filtered through Celite®, concentrated in vacuo and purified by reverse-phase HPLC eluting with a 0.1% TFA modified gradient of 5-95% MeCN in H₂O to provide N-[8-isopropoxy-2-(1-methyl-2-oxabicyclo[2.1.1]hexan-4-yl)imidazo[1,2-a]pyrazin-6-yl]-2-methoxy-pyridine-3-carboxamide 2,2,2-trifluoroacetate (25.8 mg, 36% yield). LCMS (ESI) m/z 424.3 (M+H)⁺. ¹H NMR (500 MHz, DMSO-d₆) δ ppm 1.38-1.49 (m, 9H) 1.78 (dd, J=3.97, 1.53 Hz, 2H) 2.02 (dd, J=4.27, 1.83 Hz, 2H) 3.90 (s, 2H) 4.05 (s, 3H) 5.49 (spt, J=6.21 Hz, 1H) 7.20 (dd, J=7.32, 4.88 Hz, 1H) 8.08 (s, 1H) 8.20 (dd, J=7.32, 1.83 Hz, 1H) 8.38 (dd, J=4.88, 1.83 Hz, 1H) 8.93 (s, 1H) 10.29 (s, 1H).

### Example 338: N-(8-isopropoxy-2-(1-methyl-2-oxabicyclo[2.1.1]hexan-4-yl)imidazo[1,2-a]pyrazin-6-yl)-4-methoxypyrazolo[1,5-a]pyridine-3-carboxamide

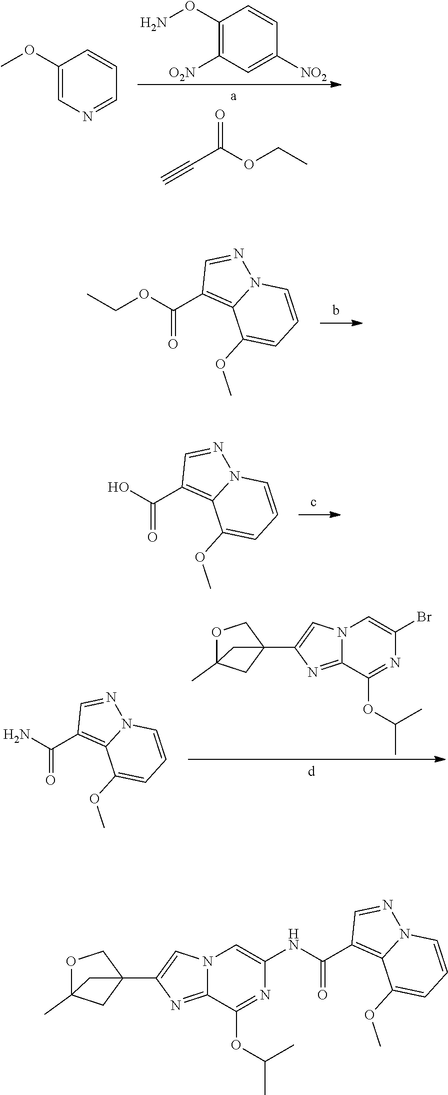

Step a: To a solution of 3-methoxypyridine (500 mg, 4.58 mmol) in MeCN (10 mL) was added 2,4-dinitrophenyl)hydroxylamine (760.26 mg, 3.82 mmol) and the reaction stirred at 40° C. for 4 h. The mixture was filtered and concentrated in vacuo to give an orange solid, 1.18 g. This was dissolved in DMF (3 mL), K₂CO₃ (529.34 mg, 3.83 mmol) and ethyl propiolate (450.87 mg, 4.60 mmol) and the reaction stirred at 25° C. for 16 h. The reaction mixture was diluted with water (30 mL) and extracted with EtOAc (100 mL×3). The combined organic extracts were washed with brine (100 mL), dried over Na₂SO₄, filtered and concentrated in vacuo. The residue was purified by column chromatography on silica gel (PE/EtOAc=10/1 to 1/1) to give ethyl 4-methoxypyrazolo[1,5-a]pyridine-3-carboxylate (490 mg, 52.29% yield) as a brown oil. ¹H NMR (500 MHz, CDCl₃) δ ppm 1.37 (t, J=7.0 Hz, 3H), 3.98 (s, 3H), 4.33 (q, J=7.0 Hz, 2H), 6.63 (d, J=7.5 Hz, 1H), 6.83-6.79 (m, 1H), 8.15 (d, J=6.5 Hz, 1H), 8.34 (s, 1H).

Step b: To a solution of ethyl 4-methoxypyrazolo[1,5-a]pyridine-3-carboxylate (490 mg, 2.22 mmol) in MeOH (5 mL) and H₂O (5 mL) was added NaOH (266.40 mg, 6.66 mmol) and the reaction stirred at 20° C. for 2 h. 1M HCl was added to neutralize the reaction, and the mixture concentrated in vacuo. The aqueous layer was concentrated by vacuum freeze drying, to give 4-methoxypyrazolo[1,5-a]pyridine-3-carboxylic acid (492 mg, crude) as a brown solid, which was used without further purification.

Step c: To a solution of 4-methoxypyrazolo[1,5-a]pyridine-3-carboxylic acid (490.04 mg, 2.55 mmol) in DCM (10 mL) was added DMF (1 drop) and SOCl₂ (1.52 g, 12.75 mmol) at 0° C. and the reaction stirred at 0° C. for 2 h, followed by a further 12 h at rt. The reaction was concentrated in vacuo, the residue dissolved in THF (10 mL) and the solution cooled to 0° C. NH₄OH (408.33 mg, 11.65 mmol) was added and the reaction stirred at 0° C. for 12 h. The reaction was evaporated under reduced pressure to give 4-methoxypyrazolo[1,5-a]pyridine-3-carboxamide, 420 mg, which was used without further purification.

Step d: Cs₂CO₃ (83.25 mg, 255.51 μmol), Pd₂dba₃ (7.80 mg, 8.52 μmol) and XantPhos (9.86 mg, 17.03 μmol) were added to a solution of 4-methoxypyrazolo[1,5-a]pyridine-3-carboxamide (32.5 mg, 170.3 μmol) and Example 336, step b: 6-bromo-8-isopropoxy-2-(1-methyl-2-oxabicyclo[2.1.1]hexan-4-yl)imidazo[1,2-a]pyrazine (30 mg, 85.17 μmol) in toluene (3 mL), the reaction mixture purged with N₂ and stirred at 110° C. for 12 h. The cooled mixture was concentrated in vacuo and the residue purified by prep-HPLC (Column: Welch Xtimate C18 150×25 mm×5 um) eluting with water/(10 mM NH₄HCO₃-MeCN from 33% to 63%) to give N-(8-isopropoxy-2-(1-methyl-2-oxabicyclo[2.1.1]hexan-4-yl)imidazo[1,2-a]pyrazin-6-yl)-4-methoxypyrazolo[1,5-a]pyridine-3-carboxamide (8.3 mg, 98.21% purity) as a brown solid. LCMS (ESI) m/z 463.3 (M+H)⁺. ¹H NMR (500 MHz, DMSO-d₆) δ ppm 1.43 (s, 3H), 1.46 (d, J=6.0 Hz, 6H), 1.78-1.76 (m, 2H), 2.02-2.01 (m, 2H), 3.89 (s, 2H), 4.21 (s, 3H), 5.52-5.47 (m, 1H), 7.11-7.07 (m, 1H), 7.15 (d, J=8.0 Hz, 1H), 8.07 (s, 1H), 8.51 (s, 1H), 8.53 (d, J=6.5 Hz, 1H), 8.92 (s, 1H), 10.58 (s, 1H).

### Example 339: 6-(difluoromethyl)-N-(8-isopropoxy-2-(tetrahydro-2H-pyran-4-yl)imidazo[1,2-a]pyrazin-6-yl)picolinamide

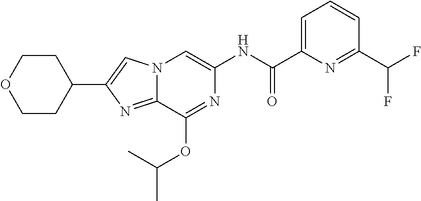 [SMILES: CC(C)Oc1nc(NC(=O)c2cccc(C(F)F)n2)cn2cc(C3CCOCC3)nc12]

6-(Difluoromethyl)-N-(8-isopropoxy-2-(tetrahydro-2H-pyran-4-yl)imidazo[1,2-a]pyrazin-6-yl)picolinamide 2,2,2-trifluoroacetate was prepared from 3-isopropoxypyrazin-2-amine, 2-bromo-1-(tetrahydro-2H-pyran-4-yl)ethan-1-one and (6-difluoromethyl)picolinamide following the method described in Example 336. LCMS (ESI) m/z 432.3 (M+H)⁺; ¹H NMR (500 MHz, DMSO-d₆) δ ppm 1.64-1.76 (m, 3H), 1.92 (br dd, J=12.82, 2.44 Hz, 3H), 2.96 (tt, J=11.52, 3.74 Hz, 1H), 3.47 (br dd, J=11.60, 9.77 Hz, 4H), 3.94 (dt, J=9.46, 2.29 Hz, 2H), 5.54-5.59 (m, 1H), 6.96-7.30 (m, 2H), 8.01-8.05 (m, 2H), 8.30-8.38 (m, 2H), 8.97 (s, 1H), 9.94 (s, 1H).

### Example 340: N-(8-cyclobutoxy-2-(1-methyl-2-oxabicyclo[2.1.1]hexan-4-yl)imidazo[1,2-a]pyrazin-6-yl)-2-cyclopropyloxazole-4-carboxamide

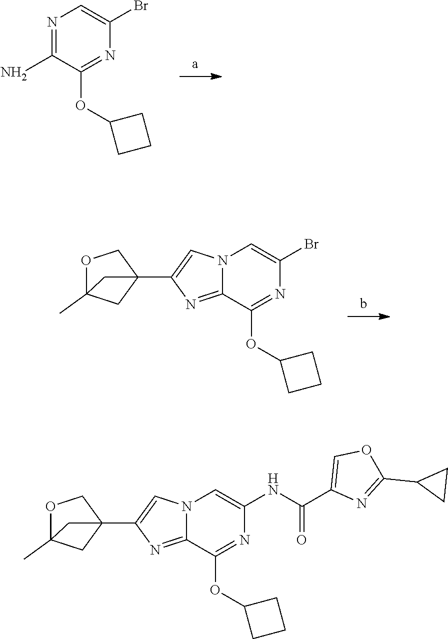 [SMILES: Nc1ncc(Br)nc1OC1CCC1 ; CC12CC(c3cn4cc(Br)nc(OC5CCC5)c4n3)(CO1)C2 ; CC12CC(c3cn4cc(NC(=O)c5coc(C6CC6)n5)nc(OC5CCC5)c4n3)(CO1)C2]

N-[8-(cyclobutoxy)-2-(1-methyl-2-oxabicyclo[2.1.1]hexan-4-yl)imidazo[1,2-a]pyrazin-6-yl]-2-cyclopropyl-oxazole-4-carboxamide 2,2,2-trifluoroacetate was prepared from 3-(cyclobutoxy)pyrazin-2-amine, 2-bromo-1-(1-methyl-2-oxabicyclo[2.1.1]hexan-4-yl)ethan-1-one and 2-cyclopropyloxazole-4-carboxamide following the same synthetic sequence described in Example 336. (17 mg, 35.55% yield) LCMS (ESI) m/z 436.2 (M+H)⁺; ¹H NMR (500 MHz, DMSO-d₆) δ ppm 1.02-1.17 (m, 4H), 1.43 (s, 3H), 1.65-1.78 (m, 1H), 1.75-1.81 (m, 2H), 1.82-1.93 (m, 1H), 1.98-2.06 (m, 2H), 2.14-2.28 (m, 3H), 3.90 (s, 2H), 5.38 (quin, J=7.48 Hz, 1H), 8.10 (s, 1H), 8.66-8.90 (m, 1H), 9.34 (s, 1H).

### Example 341: N-(8-cyclobutoxy-2-(1-methyl-2-oxabicyclo[2.2.1]heptan-4-yl)imidazo[1,2-a]pyrazin-6-yl)-2-cyclopropyloxazole-4-carboxamide

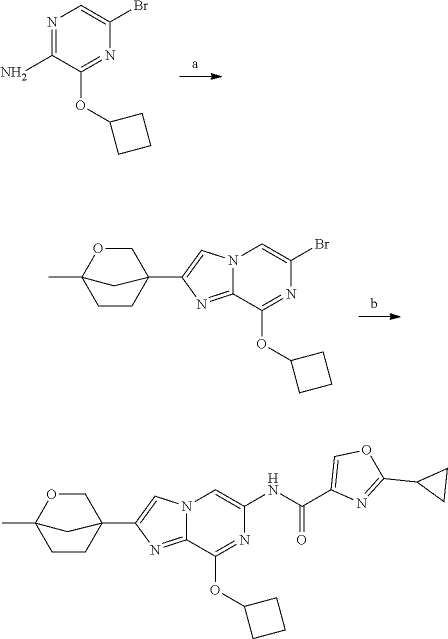 [SMILES: Nc1ncc(Br)nc1OC1CCC1 ; CC12CCC(c3cn4cc(Br)nc(OC5CCC5)c4n3)(CO1)C2 ; CC12CCC(c3cn4cc(NC(=O)c5coc(C6CC6)n5)nc(OC5CCC5)c4n3)(CO1)C2]

N-(8-cyclobutoxy-2-(1-methyl-2-oxabicyclo[2.2.1]heptan-4-yl)imidazo[1,2-a]pyrazin-6-yl)-2-cyclopropyloxazole-4-carboxamide was prepared from 3-(cyclobutoxy)pyrazin-2-amine, 2-bromo-1-(1-methyl-2-oxabicyclo[2.1.1]heptan-4-yl)ethan-1-one and 2-cyclopropyloxazole-4-carboxamide following a similar synthetic sequence to that described in Example 336 (14.90 mg, 33.15 μmol, 31.27% yield). LCMS (ESI) m/z 450.2 (M+H)⁺; ¹H NMR (500 MHz, DMSO-d₆) δ ppm 1.03-1.14 (m, 4H), 1.38 (s, 3H), 1.63-1.85 (m, 5H), 1.92-1.98 (m, 1H), 2.01-2.10 (m, 1H), 2.16-2.28 (m, 3H), 3.78 (d, J=6.71 Hz, 1H), 3.91 (dd, J=6.41, 3.36 Hz, 1H), 5.31-5.43 (m, 1H), 8.09 (s, 1H), 8.64-8.87 (m, 1H), 9.33 (s, 1H)

### Example 342: 6-(difluoromethyl)-N-(8-(pyrrolidin-1-yl)-2-(tetrahydro-2H-pyran-4-yl)imidazo[1,2-a]pyrazin-6-yl)picolinamide

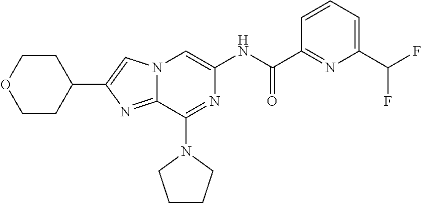 [SMILES: O=C(Nc1cn2cc(C3CCOCC3)nc2c(N2CCCC2)n1)c1cccc(C(F)F)n1]

6-(Difluoromethyl)-N-(8-(pyrrolidin-1-yl)-2-(tetrahydro-2H-pyran-4-yl)imidazo[1,2-a]pyrazin-6-yl)picolinamide 2,2,2-trifluoroacetate was prepared from 5-bromo-3-(pyrrolidin-1-yl)pyrazin-2-amine, 2-bromo-1-(tetrahydro-2H-pyran-4-yl)ethan-1-one and 6-(difluoromethyl)picolinamide following the synthetic sequence described in Example 336. (7.10 mg, 9.30% yield)¹H NMR (500 MHz, DMSO-d₆) δ ppm 1.64-1.74 (m, 3H), 1.89-2.00 (m, 8H), 2.93 (tt, J=11.29, 3.97 Hz, 1H), 3.47 (td, J=11.60, 1.83 Hz, 3H), 3.91-3.95 (m, 3H), 6.99-7.31 (m, 3H), 7.86 (s, 1H), 8.01 (dd, J=6.71, 1.83 Hz, 1H), 8.31-8.35 (m, 2H), 8.59 (s, 1H), 9.66 (s, 1H).

### Example 343: N-(7-ethoxy-2-(tetrahydro-2H-pyran-4-yl)imidazo[1,2-a]pyrimidin-6-yl)-2,3-dihydrobenzofuran-7-carboxamide

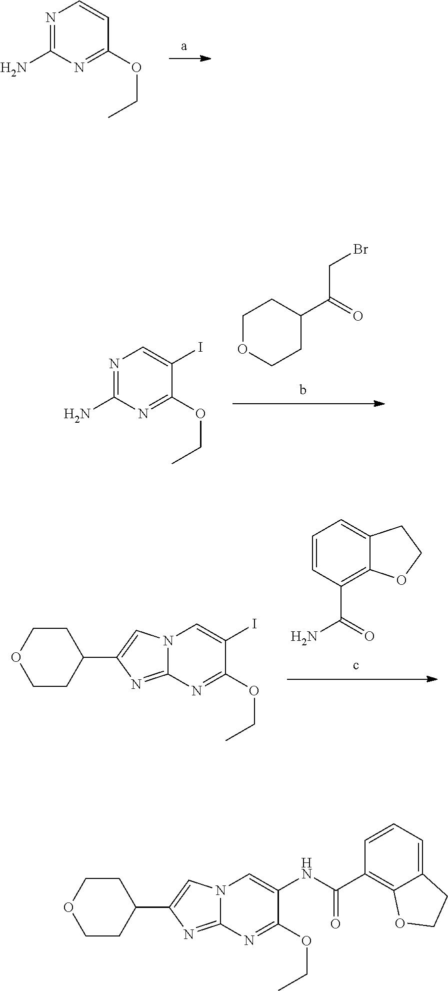

Step a: To a solution of 4-ethoxypyrimidin-2-amine (500 mg, 3.59 mmol) in DCM (15.00 mL) was added NIS (808.35 mg, 3.59 mmol) and the reaction stirred at 25° C. for 16 h. The mixture was concentrated in vacuo and the residue purified by column chromatography on silica gel using Combiflash® (PE/EtOAc=10/1 to 3/1) to give 4-ethoxy-5-iodopyrimidin-2-amine (500 mg, 52.55% yield) as white solid. LCMS (ESI) m/z 265.8 (M+H)⁺; ¹H NMR (500 MHz, CDCl₃) δ ppm: 1.40 (t, J=7.0 Hz, 3H), 4.37 (q, J=7.0 Hz, 2H), 4.89 (s, 1H), 8.24 (s, 1H).

Step b: To a solution of 4-ethoxy-5-iodopyrimidin-2-amine (400 mg, 1.51 mmol) in EtOH (10 mL) was added 2-bromo-1-(tetrahydro-2H-pyran-4-yl)ethan-1-one (468.99 mg, 2.27 mmol) and NaHCO₃ (253.71 mg, 3.02 mmol) and the reaction stirred at 85° C. for 24 h. The solution was concentrated in vacuo and the residue purified by column chromatography on silica gel using Combiflash® (PE/EtOAc=10/1 to 1/3) to give 7-ethoxy-6-iodo-2-(tetrahydro-2H-pyran-4-yl)imidazo[1,2-a]pyrimidine (130 mg, 313.51 μmol, 20.76% yield, 90% purity) as yellow solid. LCMS (ESI) m/z 374.0 (M+H)⁺;

Step c: To a solution of 7-ethoxy-6-iodo-2-(tetrahydro-2H-pyran-4-yl)imidazo[1,2-a]pyrimidine (30 mg, 80.39 μmol) in toluene (3 mL) was added 2,3-dihtdrobenzofuran-7-carboxamide (19.68 mg, 120.58 μmol), Pd₂(dba)₃ (7.36 mg, 8.04 μmol), XantPhos (9.30 mg, 16.08 μmol) and Cs₂CO₃ (52.39 mg, 160.78 μmol) and the reaction stirred at 110° C. for 12 h. The cooled mixture was concentrated in vacuo and the residue purified by prep-HPLC (Column: Phenomenex Synergi C18 150×30 mm×5 um) eluting with water (0.225% Formic acid in MeCN from 25% to 55%) to afford N-(7-ethoxy-2-(tetrahydro-2H-pyran-4-yl)imidazo[1,2-a]pyrimidin-6-yl)-2,3-dihydrobenzofuran-7-carboxamide (2.60 mg, 7.59% yield, 95.79% purity) as yellow solid. LCMS (ESI) m/z 409.0 (M+H)⁺; ¹H NMR (400 MHz, METHANOL-d4) δ: 1.64 (t, J=7.04 Hz, 3H), 1.78-1.88 (m, 2H), 2.01 (d, J=12.91 Hz, 2H), 3.07-3.12 (m, 1H), 3.38 (t, J=8.80 Hz, 2H), 3.59 (t, J=11.35 Hz, 2H), 4.06 (d, J=11.74 Hz, 2H), 4.72 (q, J=7.04 Hz, 2H), 4.88-4.93 (m, 2H), 7.06 (t, J=7.63 Hz, 1H), 7.51 (d, J=7.43 Hz, 1H), 7.75 (s, 1H), 7.88 (d, J=7.43 Hz, 1H), 9.91 (s, 1H), 10.40 (s, 1H).

### Example 344: 6-(difluoromethyl)-N-(7-ethoxy-2-(1-methyl-2-oxabicyclo[2.1.1]hexan-4-yl)imidazo[1,2-a]pyrimidin-6-yl)picolinamide

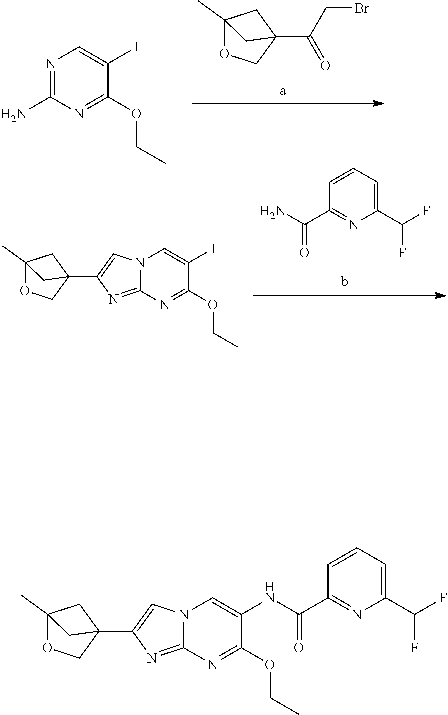 [SMILES: CCOc1nc(N)ncc1I ; CC12CC(C(=O)CBr)(CO1)C2 ; CCOc1nc2nc(C34COC(C)(C3)C4)cn2cc1I ; NC(=O)c1cccc(C(F)F)n1 ; CCOc1nc2nc(C34COC(C)(C3)C4)cn2cc1NC(=O)c1cccc(C(F)F)n1]

Step a: To a solution of 4-ethoxy-5-iodopyrimidin-2-amine (100 mg, 377.29 μmol) and 2-bromo-1-(1-methyl-2-oxabicyclo[2.1.1]hexan-4-yl)ethan-1-one (107.45 mg, 490.47 μmol) in t-BuOH (1.50 mL) was added NaHCO₃ (63.39 mg, 754.57 μmol) and the reaction stirred at 100° C. for 16 h. The mixture was diluted with water (50 mL) and extracted with EtOAc (100 mL×3). The combined organic layer was washed with brine (50 mL) and dried over Na₂SO₄, filtered. The filtrate was concentrated in vacuo to give the residue, which was purified by column chromatography using Combiflash® (PE/EtOAc=5/1 to 1/5) to give 7-ethoxy-6-iodo-2-(1-methyl-2-oxabicyclo[2.1.1]hexan-4-yl)imidazo[1,2-a]pyrimidine (20 mg, 10.32% yield) as yellow solid. LCMS (ESI) m/z 386.0 (M+H)⁺;

Step b: To a solution of 7-ethoxy-6-iodo-2-(1-methyl-2-oxabicyclo[2.1.1]hexan-4-yl)imidazo[1,2-a]pyrimidine (34 mg, 88.27 μmol) in toluene (2 mL) was added 6-(difluoromethyl)picolinamide (22.79 mg, 132.40 μmol), XantPhos (10.21 mg, 17.65 μmol), Cs₂CO₃ (57.52 mg, 176.53 μmol) and Pd₂(dba)₃ (8.08 mg, 8.83 μmol) under N₂ and the reaction stirred at 100° C. for 16 h. The cooled reaction mixture was filtered and the filtrate was purified by prep-HPLC (Column: Welch Xtimate C18 150×25 mm×5 m) eluting with water (10 mm NH₄HCO₃-MeCN from 42 to 72%) to give 6-(difluoromethyl)-N-(7-ethoxy-2-(1-methyl-2-oxabicyclo[2.1.1]hexan-4-yl)imidazo[1,2-a]pyrimidin-6-yl)picolinamide (15.40 mg, 40.63% yield) as an off-white solid. LCMS (ESI) m/z 430.1 (M+H)⁺; ¹H NMR (500 MHz, METHANOL-d₄) δ ppm: 1.51 (s, 1H), 1.57 (t, J=7.0 Hz, 3H), 1.87 (dd, J=1.5 Hz, 5.0 Hz, 2H), 2.11 (dd, J=1.5 Hz, 4.5 Hz, 2H), 4.65 (t, J=7.0 Hz, 2H), 4.01 (s, 2H), 6.76-6.99 (m, 1H), 7.53 (s, 1H), 7.96 (d, J=8.0 Hz, 1H), 8.26 (t, J=8.0 Hz, 1H), 8.38 (d, J=7.5 Hz, 1H), 9.65 (s, 1H).

### Example 345: N-(7-isopropoxy-2-(1-methyl-2-oxabicyclo[2.1.1]hexan-4-yl)imidazo[1,2-a]pyrimidin-6-yl)pyrazolo[1,5-a]pyridine-7-carboxamide

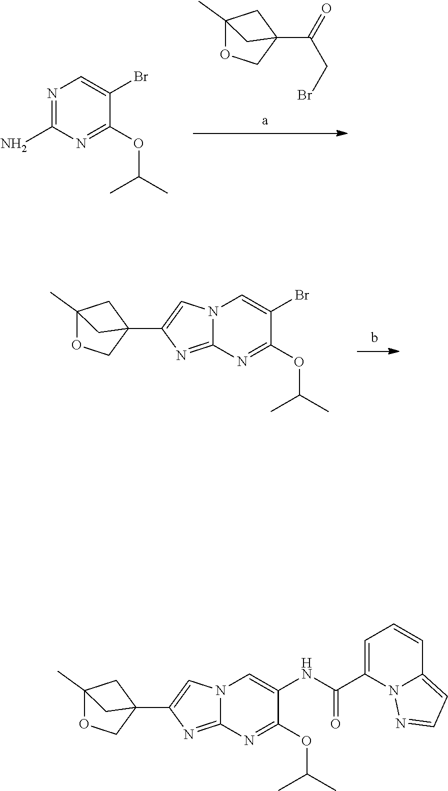

Step a: A mixture of 5-bromo-4-isopropoxy-pyrimidin-2-amine (1.38 g, 5.93 mmol), 2-bromo-1-(1-methyl-2-oxabicyclo[2.1.1]hexan-4-yl)ethan-1-one (1.56 g, 7.12 mmol) and NaHCO₃ (1.50 g, 17.80 mmol) in EtOH (17.80 mL) was heated at 80° C. overnight. The reaction mixture was cooled to rt, silica gel added, the mixture concentrated in vacuo and purified by column chromatography on silica gel to obtain 6-bromo-7-isopropoxy-2-(1-methyl-2-oxabicyclo[2.1.1]hexan-4-yl)imidazo[1,2-a]pyrimidine (330 mg, 15.79% yield). LCMS (ESI) m/z 351.9 (M+H)⁺; ¹H NMR (400 MHz, CDCl₃) δ ppm 1.44 (d, J=6.27 Hz, 6H), 1.53 (s, 3H), 1.93 (dd, J=4.52, 1.76 Hz, 2H), 2.08 (dd, J=4.52, 1.76 Hz, 2H), 4.06 (s, 2H), 5.43-5.62 (m, 1H), 7.11 (s, 1H), 8.35 (s, 1H).

Step b: A vial charged with 6-bromo-7-isopropoxy-2-(1-methyl-2-oxabicyclo[2.1.1]hexan-4-yl)imidazo[1,2-a]pyrimidine (50 mg, 141.95 μmol), Cs₂CO₃ (92.50 mg, 283.90 μmol), XantPhos Pd G3; DCM (14.67 mg, 14.19 μmol) and pyrazolo[1,5-a]pyridine-7-carboxamide (45.75 mg, 283.90 μmol) was purged with N₂ and closed with a screw cap with septa. Toluene (2.84 mL) was added at rt and the vial sealed and heated at 100° C. overnight. The reaction was cooled to rt, filtered and concentrated in vacuo. The residue was purified by TFA modified mass directed HPLC to obtain N-(7-isopropoxy-2-(1-methyl-2-oxabicyclo[2.1.1]hexan-4-yl)imidazo[1,2-a]pyrimidin-6-yl)pyrazolo[1,5-a]pyridine-7-carboxamide 2,2,2-trifluoroacetate (15.60 mg, 20.15% yield). LCMS (ESI) m/z 433.0 (M+H)⁺. ¹H NMR (500 MHz, DMSO-d₆) δ ppm 1.45 (s, 3H) 1.59 (d, J=6.10 Hz, 6H), 1.84 (br d, J=3.05 Hz, 2H), 2.11 (br d, J=1.83 Hz, 2H), 3.92 (s, 2H), 5.30-5.56 (m, 1H), 7.03 (d, J=2.44 Hz, 1H), 7.55 (dd, J=8.85, 7.02 Hz, 1H) 7.93-8.06 (m, 1H) 8.16 (dd, J=8.55, 1.22 Hz, 1H), 8.36 (d, J=2.44 Hz, 1H), 9.99 (br s, 1H), 13.66 (br s, 1H).

### Example 346: N-(7-isopropoxy-2-(1-methyl-2-oxabicyclo[2.1.1]hexan-4-yl)imidazo[1,2-a]pyrimidin-6-yl)-2-methoxynicotinamide

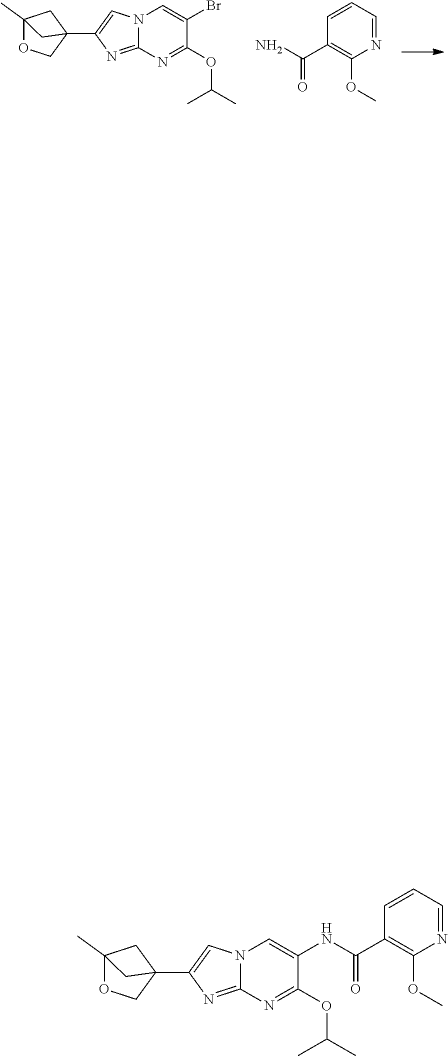 [SMILES: CC(C)Oc1nc2nc(C34COC(C)(C3)C4)cn2cc1Br ; COc1ncccc1C(N)=O ; COc1ncccc1C(=O)Nc1cn2cc(C34COC(C)(C3)C4)nc2nc1OC(C)C]

A vial charged with Example 346, step a: 6-bromo-7-isopropoxy-2-(1-methyl-2-oxabicyclo[2.1.1]hexan-4-yl)imidazo[1,2-a]pyrimidine (100 mg, 283.91 μmol), Cs₂CO₃ (185.0 mg, 567.82 μmol), XantPhos Pd G3; DCM (29.3 mg, 28.4 μmol), and 2-methoxynicotinamide (86.39 mg, 567.82 μmol) was purged with N₂ and closed with a screw cap with septa. Toluene (2.84 mL) was added at rt and the vial sealed and heated at 80° C. overnight. The cooled mixture was filtered, concentrated in vacuo, and the residue purified by TFA modified mass directed HPLC to give N-(7-isopropoxy-2-(1-methyl-2-oxabicyclo[2.1.1]hexan-4-yl)imidazo[1,2-a]pyrimidin-6-yl)-2-methoxynicotinamide 2,2,2-trifluoroacetate (66.50 mg, 43.58% yield). LCMS (ESI) m/z 424.1 (M+H)⁺. ¹H NMR (500 MHz, METHANOL-d₄) δ ppm 1.54 (s, 3H) 1.65 (d, J=6.10 Hz, 6H) 1.98 (dd, J=4.27, 1.83 Hz, 2H) 2.20 (dd, J=4.58, 1.53 Hz, 2H), 4.05 (s, 2H) 4.30 (s, 3H) 5.70 (dt, J=12.36, 6.33 Hz, 1H) 7.27 (dd, J=7.33, 4.88 Hz, 1H) 7.87 (s, 1H) 8.46 (dd, J=4.88, 1.83 Hz, 1H) 8.59 (dd, J=7.32, 1.83 Hz, 1H) 10.00 (s, 1H) 10.55-10.68 (m, 1H)

### Intermediate 23: 6-iodo-7-isopropoxy-2-(1-methyl-2-oxabicyclo[2.1.1]hexan-4-yl)imidazo[1,2-a]pyrimidine

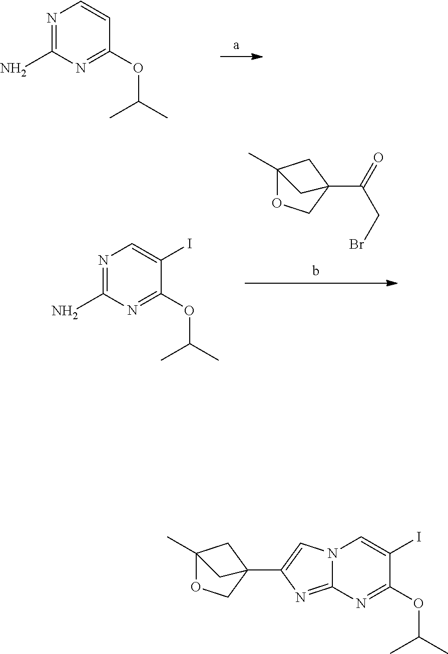 [SMILES: CC(C)Oc1ccnc(N)n1 ; CC(C)Oc1nc(N)ncc1I ; CC12CC(C(=O)CBr)(CO1)C2 ; CC(C)Oc1nc2nc(C34COC(C)(C3)C4)cn2cc1I]

Step a: To a solution of 4-isopropoxypyrimidin-2-amine (800 mg, 5.22 mmol) in MeCN (50 mL) was added 1-iodopyrrolidine-2,5-dione (1.41 g, 6.26 mmol) and the reaction stirred at 30° C. for 16 h. The mixture was concentrated in vacuo and the residue was purified by silica gel chromatography (EtOAc/PE=30%) to give 5-iodo-4-isopropoxypyrimidin-2-amine (1.10 g, 3.55 mmol, 68.01% yield) as sticky oil. LCMS (ESI) m/z 279.8 (M+H)⁺;

Step b: To a mixture of 5-iodo-4-isopropoxypyrimidin-2-amine (1.00 g, 3.58 mmol) and 2-bromo-1-(1-methyl-2-oxabicyclo[2.1.1]hexan-4-yl)ethan-1-one (997 mg, 4.55 mmol) in t-BuOH (2 mL) was added NaHCO₃ (602 mg, 7.17 mmol) and the reaction stirred at 100° C. for 16 h. The mixture was concentrated in vacuo to give the residue, which was purified by Combiflash© (PE/EtOAc=1/1) to give 6-iodo-7-isopropoxy-2-(1-methyl-2-oxabicyclo[2.1.1]hexan-4-yl)imidazo[1,2-a]pyrimidine (1.00 g, 70% yield) as yellow solid. LCMS (ESI) m/z 399.9 (M+H)⁺; ¹H NMR (500 MHz, CDCl₃) δ ppm: 1.42 (d, J=6.0 Hz, 6H), 1.52 (s, 3H), 1.85-1.90 (m, 2H), 2.05-2.10 (m, 2H), 4.04 (s, 2H), 5.40-5.50 (m, 1H), 7.07 (s, 1H), 8.46 (s, 1H)

### Example 347: 3,3-difluoro-N-(7-isopropoxy-2-(1-methyl-2-oxabicyclo[2.1.1]hexan-4-yl)imidazo[1,2-a]pyrimidin-6-yl)-2,3-dihydrobenzofuran-7-carboxamide

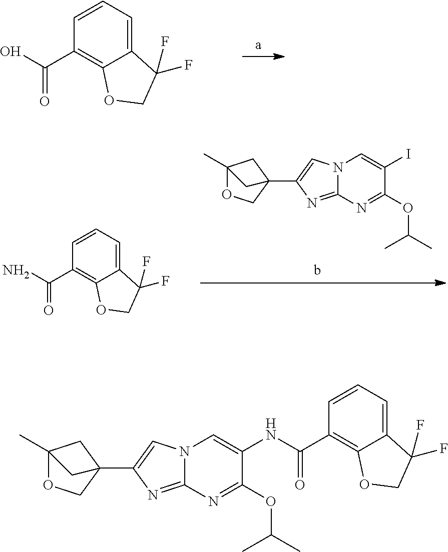 [SMILES: O=C(O)c1cccc2c1OCC2(F)F ; NC(=O)c1cccc2c1OCC2(F)F ; CC(C)Oc1nc2nc(C34COC(C)(C3)C4)cn2cc1I ; CC(C)Oc1nc2nc(C34COC(C)(C3)C4)cn2cc1NC(=O)c1cccc2c1OCC2(F)F]

Step a: To a solution of 3,3-difluoro-2,3-dihydrobenzofuran-7-carboxylic acid (70 mg, 0.350 mmol) in DCM (2 mL) was added SOCl₂ (54.09 mg, 0.455 mmol) and DMF (1 drop) at 0° C. and the reaction stirred at 10-15° C. for 12 h. The mixture was concentrated in vacuo, the residue diluted with THF (2 mL), NH₄OH (713.95 mg, 5.71 mmol) added and the reaction stirred at 15° C. for 1 h. The mixture was diluted with water (30 mL) and extracted with EtOAc (30 mL×3). The combined organic extracts were washed with brine (50 mL) and dried over Na₂SO₄ and filtered. The filtrate was evaporated under reduced pressure to give 3,3-difluoro-2,3-dihydrobenzofuran-7-carboxamide (50 mg, 71.78% yield) as a white solid. ¹H NMR (500 MHz, CDCl₃) δ ppm 4.80 (t, J=15.0 Hz, 2H), 5.81 (br s, 1H), 7.24 (t, J=8.0 Hz, 1H), 7.72 (d, J=7.5 Hz, 1H), 8.29 (d, J=7.0 Hz, 1H).

Step b: To a solution of Intermediate 23: 6-iodo-7-isopropoxy-2-(1-methyl-2-oxabicyclo[2.1.1]hexan-4-yl)imidazo[1,2-a]pyrimidine (50 mg, 125 μmol) and 3,3-difluoro-2,3-dihydrobenzofuran-7-carboxamide (37.41 mg, 187.9 μmol) in toluene (2 mL) were added XantPhos (14.49 mg, 25.1 μmol), Pd₂(dba)₃ (11.47 mg, 12.5 μmol) and Cs₂CO₃ (81.61 mg, 250.5 μmol), the reaction degassed with N₂ and stirred at 110° C. for 6 h. The cooled mixture was diluted with water (30 mL) and extracted with EtOAc (30 mL×3). The combined organic extracts were washed with brine (50 mL) and dried over Na₂SO₄ and filtered. The filtrate was concentrated in vacuo and the residue was purified by base-mediated prep-HPLC to give 3,3-difluoro-N-(7-isopropoxy-2-(1-methyl-2-oxabicyclo[2.1.1]hexan-4-yl)imidazo[1,2-a]pyrimidin-6-yl)-2,3-dihydrobenzofuran-7-carboxamide (20 mg, 33.94% yield) as a white solid. LCMS (ESI) m/z 471.1 (M+H)⁺. ¹H NMR (500 MHz, METHANOL-d₄) δ ppm 1.49 (s, 3H), 1.54 (d, J=6.0 Hz, 6H), 1.86-1.83 (m, 2H), 2.10-2.06 (m, 2H), 4.02-3.98 (m, 2H), 5.03 (t, J=8.0 Hz, 2H), 5.48 (d, J=12.0 Hz, 1H), 7.29 (t, J=8.0 Hz, 1H), 7.45 (s, 1H), 7.79 (d, J=8.0 Hz, 1H), 8.22 (d, J=8.0 Hz, 1H), 9.61 (s, 1H),

### Example 348: N-(7-isopropoxy-2-(1-methyl-2-oxabicyclo[2.1.1]hexan-4-yl)imidazo[1,2-a]pyrimidin-6-yl)-[1,2,4]triazolo[1,5-a]pyridine-5-carboxamide

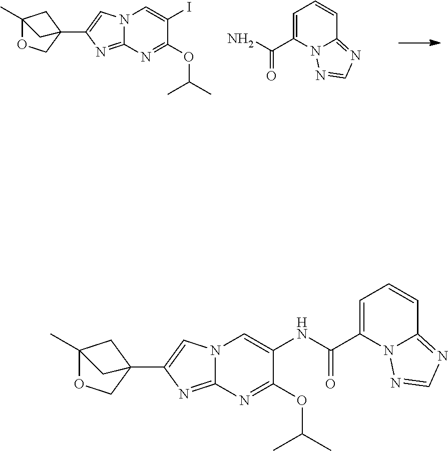 [SMILES: CC(C)Oc1nc2nc(C34COC(C)(C3)C4)cn2cc1I ; NC(=O)c1cccc2ncnn12 ; CC(C)Oc1nc2nc(C34COC(C)(C3)C4)cn2cc1NC(=O)c1cccc2ncnn12]

To a solution of Intermediate 23: 6-iodo-7-isopropoxy-2-(1-methyl-2-oxabicyclo[2.1.1]hexan-4-yl)imidazo[1,2-a]pyrimidine (30 mg, 75 μmol) and [1,2,4]triazolo[1,5-a]pyridine-5-carboxamide (18.28 mg, 112.7 mmol) in toluene (3 mL), were added Pd₂(dba)₃ (6.88 mg, 7 μmol), XantPhos (8.70 mg, 150.3 μmol) and Cs₂CO₃ (48.97 mg, 150.3 μmol), the reaction degassed with N₂ and then stirred at 110° C. for 6 h. The cooled mixture was diluted with water (30 mL), the layers separated and the aqueous layer extracted with EtOAc (30 mL×3). The combined organic layers were washed with brine (50 mL), dried over Na₂SO₄, filtered and concentrated in vacuo. The residue was purified by prep-HPLC (Welch Xtimate C18 150×30 mm×5 μm column), eluting with water (NH₄HCO₃)-MeCN from 25% to 55%), to give N-(7-isopropoxy-2-(1-methyl-2-oxabicyclo[2.1.1]hexan-4-yl)imidazo[1,2-a]pyrimidin-6-yl)-[1,2,4]triazolo[1,5-a]pyridine-5-carboxamide (10.60 mg, 32.55% yield) as an off-white solid. LCMS (ESI) m/z 471.1 (M+H)⁺. ¹H NMR (500 MHz, METHANOL-d₄) δ ppm 1.50 (s, 3H), 1.61 (d, J=6.5 Hz, 6H), 1.86 (d, J=4.5 Hz, 2H), 2.10 (d, J=4.5 Hz, 2H), 4.01 (s, 2H), 5.56-5.58 (m, 1H), 7.53 (s, 1H), 7.94-7.96 (m, 1H), 8.12 (d, J=8.5, 1H), 8.26 (d, J=7.0, 1H), 8.73 (s, 1H), 9.78 (s, 1H).

### Example 349: 4-fluoro-N-(7-isopropoxy-2-(1-methyl-2-oxabicyclo[2.1.1]hexan-4-yl)imidazo[1,2-a]pyrimidin-6-yl)pyrazolo[1,5-a]pyridine-3-carboxamide

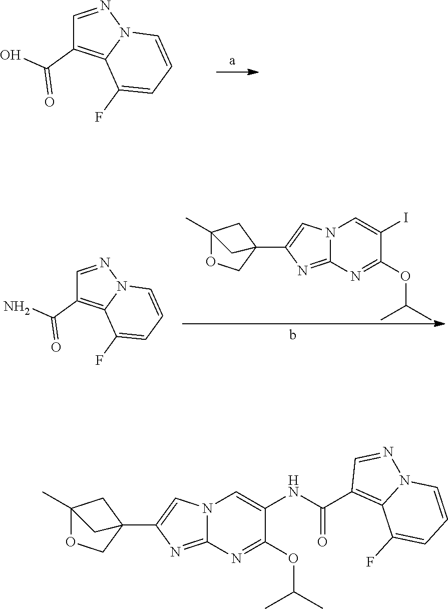 [SMILES: O=C(O)c1cnn2cccc(F)c12 ; NC(=O)c1cnn2cccc(F)c12 ; CC(C)Oc1nc2nc(C34COC(C)(C3)C4)cn2cc1I ; CC(C)Oc1nc2nc(C34COC(C)(C3)C4)cn2cc1NC(=O)c1cnn2cccc(F)c12]

Step a: To a solution of 4-fluoropyrazolo[1,5-a]pyridine-3-carboxylic acid (110 mg, 610.64 μmol) in DCM (10 mL) was added DMF (1 drop) and SOCl₂ (363.24 mg, 3.05 mmol) at 0° C. and the reaction stirred at 0° C. for 2 h, followed by a further 12 h at 25° C. The reaction was concentrated in vacuo, the residue dissolved in THF (10 mL) and the solution cooled to 0° C. NH₄OH (21.35 mg, 609.33 μmol) was added and the reaction stirred at 0° C. for 12 h. The reaction was concentrated in vacuo to give 4-fluoropyrazolo[1,5-a]pyridine-3-carboxamide (85 mg, crude)

Step b: Cs₂CO₃ (73.45 mg, 225.42 μmol), Pd₂dba₃ (6.88 mg, 7.51 μmol) and XantPhos (8.70 mg, 15.03 μmol) were added sequentially to a solution of 4-fluoropyrazolo[1,5-a]pyridine-3-carboxamide (26.92 mg, 150.28 μmol) and Intermediate 23: 6-iodo-7-isopropoxy-2-(1-methyl-2-oxabicyclo[2.1.1]hexan-4-yl)imidazo[1,2-a]pyrimidine (30 mg, 75.14 μmol) in toluene (3.00 mL) and the mixture purged with N₂. The reaction was stirred at 110° C. for 12 h, then concentrated in vacuo. The residue was purified by prep-HPLC (Column: Welch Xtimate C18 150×25 mm×5 um) eluting with water/(10 mM NH₄HCO₃-MeCN from 33% to 66%) to give 4-fluoro-N-(7-isopropoxy-2-(1-methyl-2-oxabicyclo[2.1.1]hexan-4-yl)imidazo[1,2-a]pyrimidin-6-yl)pyrazolo[1,5-a]pyridine-3-carboxamide (11.30 mg, 33.39% yield) as a brown solid. LCMS (ESI) m/z 451.2 (M+H)⁺. ¹H NMR (500 MHz, METHANOL-d₄) δ ppm: 1.49-1.53 (m, 9H), 1.84-1.86 (m, 2H), 2.08-2.10 (m, 2H), 3.99 (s, 2H), 5.52-5.58 (m, 1H), 7.08-7.11 (m, 1H), 7.37-7.42 (m, 1H), 7.47 (s, 1H), 8.53 (s, 1H), 8.61 (d, J=7.0 Hz, 1H), 9.55 (s, 1H).

### Example 350: N-(7-cyclopropoxy-2-(1-methyl-2-oxabicyclo[2.1.1]hexan-4-yl)imidazo[1,2-a]pyrimidin-6-yl)-2-methoxybenzamide

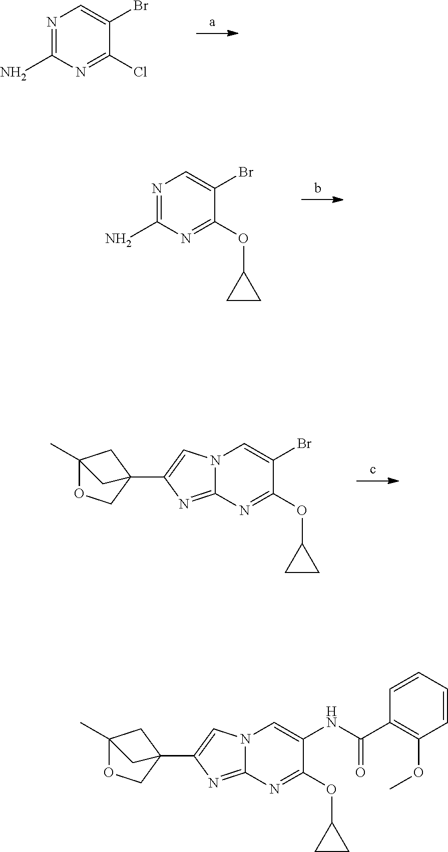 [SMILES: Nc1ncc(Br)c(Cl)n1 ; Nc1ncc(Br)c(OC2CC2)n1 ; CC12CC(c3cn4cc(Br)c(OC5CC5)nc4n3)(CO1)C2 ; COc1ccccc1C(=O)Nc1cn2cc(C34COC(C)(C3)C4)nc2nc1OC1CC1]

Step a: Cyclopropanol (1.67 g, 28.79 mmol) and Cs₂CO₃ (12.51 g, 38.38 mmol) were added to 5-bromo-4-chloropyrimidin-2-amine (4.00 g, 19.19 mmol) in DMF (47.98 mL) and the reaction heated at 70° C. for 2 h. The cooled solution was diluted with brine, extracted with EtOAc, dried and concentrated in vacuo. The residue was purified by column chromatography to give 5-bromo-4-cyclopropoxypyrimidin-2-amine (2.00 g, 8.69 mmol, 45.30% yield). LCMS (ESI) m/z 231.9 (M+H)⁺.

Step b: A mixture of 5-bromo-4-cyclopropoxypyrimidin-2-amine (600 mg, 2.61 mmol), 2-bromo-1-(1-methyl-2-oxabicyclo[2.1.1]hexan-4-yl)ethan-1-one (571.36 mg, 2.61 mmol) and NaHCO₃ (657.30 mg, 7.83 mmol) in EtOH (6.53 mL) was heated at 80° C. overnight. The reaction was cooled to rt, silica gel added, the mixture concentrated in vacuo and purified by dry load silica gel column chromatography to obtain 6-bromo-7-cyclopropoxy-2-(1-methyl-2-oxabicyclo[2.1.1]hexan-4-yl)imidazo[1,2-a]pyrimidine (300 mg, 856.63 μmol, 32.82% yield). LCMS (ESI) m/z 349.9 (M+H)⁺.

Step c: A mixture of 6-bromo-7-cyclopropoxy-2-(1-methyl-2-oxabicyclo[2.1.1]hexan-4-yl)imidazo[1,2-a]pyrimidine (50 mg, 142.77 μmol), Cs₂CO₃ (93.04 mg, 285.54 μmol), XantPhos Pd G3:DCM (14.75 mg, 14.28 μmol), and 2-methoxybenzamide (43.16 mg, 285.54 μmol) was purged with N₂, toluene (2.84 mL) added, the reaction vessel sealed and the reaction heated at 100° C. overnight. The cooled mixture was filtered, concentrated in vacuo, and the residue purified by reverse phase HPLC to give N-(7-cyclopropoxy-2-(1-methyl-2-oxabicyclo[2.1.1]hexan-4-yl)imidazo[1,2-a]pyrimidin-6-yl]-2-methoxybenzamide trifluoroacetate (27.10 mg, 50.80 μmol, 35.58% yield). LCMS (ESI) m/z 421.1 (M+H)⁺.

### Example 351: 6-(difluoromethyl)-N-(3-fluoro-8-methoxy-2-(tetrahydro-2H-pyran-4-yl)imidazo[1,2-a]pyridin-6-yl)picolinamide

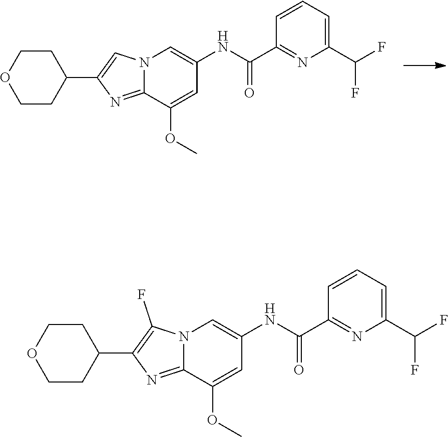 [SMILES: COc1cc(NC(=O)c2cccc(C(F)F)n2)cn2cc(C3CCOCC3)nc12 ; COc1cc(NC(=O)c2cccc(C(F)F)n2)cn2c(F)c(C3CCOCC3)nc12]

A mixture of Example 305: 6-(difluoromethyl)-N-(8-methoxy-2-(tetrahydro-2H-pyran-4-yl)imidazo[1,2-a]pyridin-6-yl)picolinamide (120 mg, 298.22 μmol), DMAP (36.43 mg, 298.22 μmol) and 1-chloromethyl-4-fluoro-1,4-diazoniabicyclo[2.2.2]octane bis(tetrafluoroborate) (211.29 mg, 596.44 μmol) in chloroform (1.79 mL) and water (1.19 mL) was stirred at 0° C. for 2 h, then allowed to warm to rt and stirred overnight. The reaction was quenched with sat. aq. Na₂CO₃, extracted with EtOAc, concentrated in vacuo and the crude product purified by TFA modified mass directed HPLC to give 6-(difluoromethyl)-N-(3-fluoro-8-methoxy-2-(tetrahydro-2H-pyran-4-yl)imidazo[1,2-a]pyridin-6-yl)picolinamide 2,2,2-trifluoroacetate (28.80 mg, 22.97%). LCMS (ESI) m/z 421.3 (M+H)⁺; ¹H NMR (400 MHz, CDCl₃) δ ppm 1.99-2.10 (m, 4H) 3.27-3.37 (m, 1H), 3.55-3.63 (m, 2H), 4.09-4.17 (m, 5H), 6.62-6.91 (m, 1H), 6.99-7.04 (m, 1H), 7.94 (d, J=7.78 Hz, 1H), 8.15-8.20 (m, 1H), 8.40-8.45 (m, 1H), 9.09 (s, 1H), 9.99 (br s, 1H).

### Example 352: 6-(difluoromethyl)-N-(3-fluoro-7-isopropoxy-2-(tetrahydro-2H-pyran-4-yl)imidazo[1,2-a]pyridin-6-yl)picolinamide

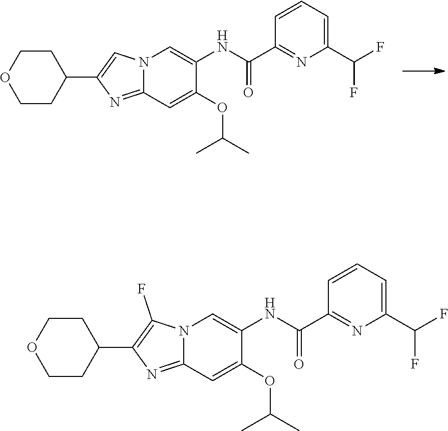 [SMILES: CC(C)Oc1cc2nc(C3CCOCC3)cn2cc1NC(=O)c1cccc(C(F)F)n1 ; CC(C)Oc1cc2nc(C3CCOCC3)c(F)n2cc1NC(=O)c1cccc(C(F)F)n1]

A solution of 1-chloromethyl-4-fluoro-1,4-diazoniabicyclo[2.2.2]octane bis(tetrafluoroborate) (41.15 mg, 116.16 μmol) in THF (291.43 uL) and water (291.43 uL) was added to 6-(difluoromethyl)-N-(7-isopropoxy-2-(tetrahydro-2H-pyran-4-yl)imidazo[1,2-a]pyridin-6-yl)picolinamide (Example 167) (50 mg, 116.16 μmol) in MeCN (1.36 ml) at 0° C., the reaction allowed to warm to rt and stirred overnight. The mixture was quenched with sat. aq. Na₂CO₃, extracted with EtOAc and the combined organic extracts concentrated in vacuo. The residue was purified by HPLC using an XSelect CSH Prep C18 OBD 5 μm 50×100 mm column, eluting with water/(0.2% NH₄OH-MeCN from 20 to 75%) to give 6-(difluoromethyl)-N-(3-fluoro-7-isopropoxy-2-(tetrahydro-2H-pyran-4-yl)imidazo[1,2-a]pyridin-6-yl)picolinamide, 0.5 mg. LCMS (ESI) m/z 449.3 (M+H)⁺; ¹H NMR (600 MHz, CDCl₃) δ ppm 1.52 (d, J=5.87 Hz, 8H), 1.96-2.07 (m, 5H), 3.09 (br t, J=11.01 Hz, 1H), 3.57 (td, J=11.56, 2.57 Hz, 2H), 4.03-4.10 (m, 2H), 4.73 (dt, J=11.74, 5.87 Hz, 1H), 6.57-6.78 (m, 1H), 6.94 (br s, 1H), 7.85 (d, J=7.34 Hz, 1H), 8.12 (t, J=7.70 Hz, 1H), 8.39 (d, J=8.07 Hz, 1H), 9.29 (s, 1H), 10.62 (s, 1H).

### Example 353: N-(3-chloro-7-isopropoxy-2-(tetrahydro-2H-pyran-4-yl)imidazo[1,2-a]pyridin-6-yl)-6-(difluoromethyl)picolinamide

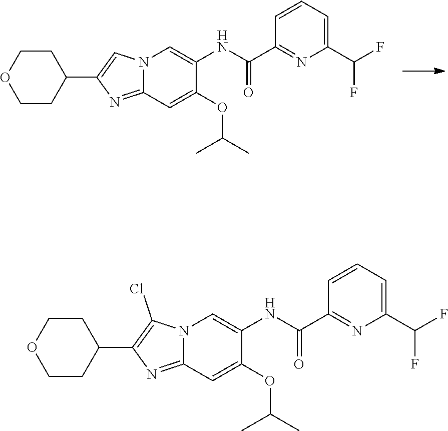 [SMILES: CC(C)Oc1cc2nc(C3CCOCC3)cn2cc1NC(=O)c1cccc(C(F)F)n1 ; CC(C)Oc1cc2nc(C3CCOCC3)c(Cl)n2cc1NC(=O)c1cccc(C(F)F)n1]

1-Chloropyrrolidine-2,5-dione (15.51 mg, 116.16 μmol) was added to a solution of 6-(difluoromethyl)-N-(7-isopropoxy-2-(tetrahydro-2H-pyran-4-yl)imidazo[1,2-a]pyridin-6-yl)picolinamide (Example 167) (50 mg, 116.16 μmol) in THF (580 uL) and EtOH (580 uL) at 0° C., and the reaction stirred at rt for 1.5 h. The reaction was quenched with aq. sat. NaHCO₃, extracted with EtOAc (3×), the combined organic extracts washed brine, dried over MgSO₄, filtered and concentrated in vacuo. The crude was purified by TFA modified mass-directed HPLC to give N-(3-chloro-7-isopropoxy-2-tetrahydropyran-4-ylimidazo[1,2-a]pyridin-6-yl)-6-(difluoromethyl)picolinamide 2,2,2-trifluoroacetate (20.70 mg, 35.40 μmol, 30.47% yield). LCMS (ESI) m/z 465.2 (M+H)⁺; ¹H NMR (500 MHz, DMSO-d₆) δ ppm 1.46 (d, J=6.10 Hz, 6H), 1.74 (br dd, J=12.82, 1.83 Hz, 2H), 1.84-1.94 (m, 2H), 3.10 (br t, J=11.60 Hz, 1H), 3.44-3.53 (m, 1H), 3.97 (br dd, J=11.29, 3.36 Hz, 2H), 4.96-5.05 (m, 1H), 7.03-7.29 (m, 1H), 7.36 (s, 1H), 8.00-8.06 (m, 1H), 8.34-8.38 (m, 1H), 9.35 (s, 1H), 10.66 (s, 1H)

Example 354: N-(3-chloro-7-ethoxy-2-(2-hydroxypropan-2-yl)imidazo[1,2-a]pyridin-6-yl)-6-(difluoromethyl)picolinamide was prepared by reacting Example 100 under to similar conditions and using a similar method to that described in Example 353, 3.80 mg, 34.9%. LCMS (ESI) m/z 391.3 (M+H); ¹H NMR (500 MHz, DMSO-d₆) δ ppm 1.54 (t, J=7.02 Hz, 4H) 1.58 (s, 8H) 4.31-4.37 (m, 2H) 7.07-7.20 (m, 1H) 7.23-7.29 (m, 2H) 8.01-8.07 (m, 1H) 8.34-8.39 (m, 2H) 9.36 (s, 1H) 10.64 (s, 1H)

The following compounds were synthesized according to the procedures similar to those described in Examples 1 to 354

Example 355: 6-(difluoromethyl)-N-(2-((tetrahydrofuran-3-yl)methyl)imidazo[1,2-a]pyridin-6-yl)picolinamide; LCMS (ESI) m/z 373.0 (M+H)⁺; ¹H NMR (500 MHz, CHLOROFORM-d) δ ppm 1.64-1.76 (m, 1H), 2.02-2.13 (m, 1H), 2.66-2.92 (m, 3H), 3.54 (dd, J=8.5, 6.1 Hz, 1H), 3.78 (q, J=7.6 Hz, 1H), 3.91 (dt, J=8.2, 6.0 Hz, 2H), 6.73 (t, J=55.1 Hz, 1H), 7.09 (dd, J=9.6, 2.1 Hz, 1H), 7.40 (s, 1H), 7.53 (d, J=9.4 Hz, 1H), 7.86 (d, J=7.8 Hz, 1H), 8.11 (t, J=7.8 Hz, 1H), 8.40 (d, J=7.8 Hz, 1H), 9.23-9.36 (m, 1H), 9.71 (s, 1H).

Example 356: 6-(difluoromethyl)-N-(2-(tetrahydro-2H-pyran-4-yl)imidazo[1,2-a]pyridin-6-yl)picolinamide; LCMS (ESI) m/z 373.2 (M+H)⁺; ¹H NMR (400 MHz, CHLOROFORM-d) δ ppm 1.24-1.27 (m, 1H), 1.85-1.87 (m, 1H), 1.96-2.11 (m, 2H), 3.01-3.08 (m, 1H), 3.57 (t, J=11.6 Hz, 2H), 4.07 (d, J=11.6 Hz, 2H), 6.73 (t, J=55.1 Hz, 1H), 7.18 (d, J=9.2 Hz, 1H), 7.40 (s, 1H), 7.64 (d, J=9.2 Hz, 1H), 7.86 (d, J=7.8 Hz, 1H), 8.11 (t, J=7.8 Hz, 1H), 8.40 (d, J=7.8 Hz, 1H), 9.36 (s, 1H), 9.74 (s, 1H)

Example 357: 6-(difluoromethyl)-N-(2-(1-methyl-2-oxabicyclo[2.1.1]hexan-4-yl)imidazo[1,2-a]pyridin-6-yl)picolinamide; LCMS (ESI) m/z 385.0 (M+H)^{+ 1}H NMR (500 MHz, METHANOL-d₄) δ ppm: 1.51 (s, 3H), 1.89-1.92 (m, 2H), 2.13-2.14 (m, 2H), 4.04 (s, 2H), 6.90 (t, J=55.0 Hz, 1H), 7.51-7.58 (m, 2H), 7.80 (s, 1H), 7.95 (d, J=8.0 Hz, 1H), 8.24 (t, J=7.5 Hz, 1H), 8.37 (d, J=7.5 Hz, 1H), 9.32 (s, 1H)

Example 358: N-(2-(2-cyanopropan-2-yl)-7-ethoxyimidazo[1,2-a]pyridin-6-yl)-2-isopropyloxazole-4-carboxamide; LCMS (ESI) m/z 382.2 (M+H)⁺; ¹H NMR (400 MHz, CHLOROFORM-d) δ ppm 1.38 (d, J=6.9 Hz, 6H), 1.57 (t, J=7.0 Hz, 3H), 1.78 (s, 6H), 3.07-3.14 (m, 1H), 4.17 (q, J=7.0 Hz, 2H), 6.89 (s, 1H), 7.43 (s, 1H), 8.17 (s, 1H), 9.34 (s, 1H), 9.41 (s, 1H).

Example 359: N-(2-(2-cyanopropan-2-yl)-7-ethoxyimidazo[1,2-a]pyridin-6-yl)-2-(difluoromethyl)oxazole-4-carboxamide; LCMS (ESI) m/z 390.0 (M+H)⁺; ¹H NMR (400 MHz, DMSO-d₆) δ ppm 1.42 (t, J=6.9 Hz, 3H), 1.70 (s, 6H), 4.23 (q, J=6.9 Hz, 2H), 7.14 (s, 1H), 7.36 (t, J=51.9 Hz, 1H), 7.91 (s, 1H), 9.12 (s, 1H), 9.18 (s, 1H), 9.39 (s, 1H).

Example 360: N-(2-(2-cyanopropan-2-yl)-7-ethoxyimidazo[1,2-a]pyridin-6-yl)-2-cyclopropyloxazole-4-carboxamide; LCMS (ESI) m/z 380.2 (M+H)⁺; ¹H NMR (400 MHz, DMSO-d₆) δ ppm 1.00-1.04 (m, 2H), 1.11-1.16 (m, 2H), 1.45 (t, J=6.9 Hz, 3H), 1.70 (s, 6H), 2.18-2.25 (m, 1H), 4.23 (q, J=6.9 Hz, 2H), 7.28 (s, 1H), 7.91 (s, 1H), 8.68 (s, 1H), 9.27 (s, 1H), 9.29 (s, 1H).

Example 361: 6-cyclopropoxy-N-(7-ethoxy-2-(tetrahydro-2H-pyran-4-yl)imidazo[1,2-a]pyridin-6-yl)picolinamide; LCMS (ESI) m/z 423.2 (M+H)⁺; ¹H NMR (400 MHz, CHLOROFORM-d) δ ppm: 0.78-0.88 (m, 4H), 1.44 (t, J=7.0 Hz, 3H), 1.76-1.91 (m, 2H), 2.02 (d, J=13.0 Hz, 2H), 2.96 (t, J=11.7 Hz, 1H), 3.55 (t, J=11.4 Hz, 2H), 3.91-4.25 (m, 4H), 4.34-4.45 (m, 1H), 6.79-6.97 (m, 2H), 7.19 (s, 1H), 7.77 (t, J=7.8 Hz, 1H), 7.88 (d, J=7.3 Hz, 1H), 9.50 (s, 1H), 10.37 (s, 1H)

Example 362: 6-(difluoromethoxy)-N-(7-ethoxy-2-(tetrahydro-2H-pyran-4-yl)imidazo[1,2-a]pyridin-6-yl)picolinamide; LCMS (ESI) m/z 433.2 (M+H)⁺; ¹H NMR: (400 MHz, CHLOROFORM-d) δ ppm: 1.58 (t, J=7.0 Hz, 3H), 1.74-1.85 (m, 2H), 2.01 (m, 2H), 2.90-2.99 (m, 1H), 3.55 (t, J=11.4 Hz, 2H), 4.05 (m, 2H), 4.17 (q, J=7.0 Hz, 2H), 6.87 (s, 1H), 7.12 (d, J=7.8 Hz, 1H), 7.45 (s, 1H), 7.27-7.63 (t, J=75 Hz, 1H), 7.97 (t, J=7.8 Hz, 1H), 8.08 (d, J=7.8 Hz, 1H), 9.44 (s, 1H), 10.07 (s, 1H),

Example 363: N-(7-ethoxy-2-(tetrahydro-2H-pyran-4-yl)imidazo[1,2-a]pyridin-6-yl)-6-(2,2,2-trifluoroethoxy)picolinamide; LCMS (ESI) m/z 465.2 (M+H)⁺; ¹H NMR (400 MHz, METHANOL-d₄) δ ppm: 1.57 (t, J=6.9 Hz, 3H), 1.74-1.87 (m, 2H), 1.98 (d, J=13.2 Hz, 2H), 2.91-3.00 (m, 1H), 3.58 (t, J=11.6 Hz, 2H), 4.03 (d, J=11.6 Hz, 2H), 4.24 (q, J=7.1 Hz, 2H), 5.00 (q, J=8.6 Hz, 2H), 6.86 (s, 1H), 7.17 (d, J=8.1 Hz, 1H), 7.47 (s, 1H), 7.86-8.08 (m, 2H), 9.46 (s, 1H)

Example 364: N-(7-ethoxy-2-(tetrahydro-2H-pyran-4-yl)imidazo[1,2-a]pyridin-6-yl)-6-(methoxymethyl)picolinamide; LCMS (ESI) m/z 411.2 (M+H)⁺; ¹H NMR (400 MHz, CHLOROFORM-d) δ ppm: 1.59 (t, J=6.9 Hz, 3H), 1.79-1.89 (m, 2H), 2.00 (d, J=13.7 Hz, 2H), 3.52-3.63 (m, 2H), 2.94 (t, J=11.4 Hz, 1H), 3.50 (s, 3H), 4.05 (d, J=11.4 Hz, 2H), 4.17 (q, J=6.9 Hz, 2H), 4.61 (s, 2H), 6.85 (s, 1H), 7.18 (s, 1H), 7.61 (d, J=7.7 Hz, 1H), 7.91 (t, J=7.7 Hz, 1H), 8.13 (d, J=7.7 Hz, 1H), 9.41 (s, 1H), 10.60 (s, 1H)

Example 365: 6-cyano-N-(7-ethoxy-2-(tetrahydro-2H-pyran-4-yl)imidazo[1,2-a]pyridin-6-yl)picolinamide; LCMS (ESI) m/z 392.2 (M+H)⁺; ¹H NMR (400 MHz, CHLORFORM-d) δ ppm: 1.61 (t, J=7.0 Hz, 3H), 1.73-1.89 (m, 2H), 2.03 (d, J=13.5 Hz, 2H), 2.95-3.05 (m, 1H), 3.56 (t, J=11.4 Hz, 2H), 4.06 (d, J=11.5 Hz, 2H), 4.21 (t, J=6.9 Hz, 2H), 6.93 (s, 1H), 7.20 (s, 1H), 7.88 (d, J=7.7 Hz, 1H), 8.10 (t, J=7.9 Hz, 1H), 8.47 (d, J=7.9 Hz, 1H), 9.39 (s, 1H), 10.36 (s, 1H)

Example 366: 6-cyclopropyl-N-(7-ethoxy-2-(tetrahydro-2H-pyran-4-yl)imidazo[1,2-a]pyridin-6-yl)picolinamide; LCMS (ESI) m/z 407.3 (M+H)⁺; ¹H NMR: (400 MHz, CHLOROFORM-d): 1.01-1.19 (m, 4H), 1.62 (t, J=7.0 Hz, 3H), 1.81 (t, J=6.8 Hz, 2H), 2.00 (m, 3H), 2.95 (m, 1H), 3.54 (t, J=6.9 Hz, 2H), 4.04 (m, 2H), 4.17 (q, J=7.0 Hz, 2H), 6.90 (s, 1H), 7.10 (s, 1H), 7.38 (d, J=7.7 Hz, 1H), 7.72 (t, J=7.7 Hz, 1H), 7.96 (d, J=7.6 Hz, 1H), 9.45 (s, 1H), 10.54 (s, 1H)

Example 367: N-(7-ethoxy-2-(tetrahydro-2H-pyran-4-yl)imidazo[1,2-a]pyridin-6-yl)-6-(tetrahydrofuran-3-yl)picolinamide; LCMS (ESI) m/z 437.1 (M+H)⁺; ¹H NMR (400 MHz, METHANOL-d₄) δ ppm: 1.68 (t, J=6.9 Hz, 3H), 1.76-1.89 (m, 2H), 1.98-2.07 (m, 2H), 2.28-2.44 (m, 2H), 3.09-3.16 (m, 1H), 3.60-3.76 (m, 3H), 3.92-4.24 (m, 5H), 4.46 (q, J=7.0 Hz, 2H), 7.25 (s, 1H), 7.63 (d, J=7.6 Hz, 1H), 7.89 (s, 1H), 8.00 (t, J=7.7 Hz, 1H), 8.12 (d, J=7.4 Hz, 1H), 9.82 (s, 1H).

Example 368: N-(7-ethoxy-2-(tetrahydro-2H-pyran-4-yl)imidazo[1,2-a]pyrimidin-6-yl)-6-methoxypicolinamide; LCMS (ESI) m/z 398.0 (M+H); ¹H NMR (400 MHz, METHANOL-d₄) δ ppm: 1.51 (t, J=7.1 Hz, 3H), 1.73-1.85 (m, 2H), 1.93-2.01 (m, 2H), 2.85-2.99 (m, 1H), 3.25-3.35 (m, 5H), 4.60 (q, J=7.1 Hz, 2H), 7.04 (d, J=7.9 Hz, 1H), 7.43 (s, 1H), 7.79 (d, J=7.9 Hz, 1H), 7.87 (t, J=7.9 Hz, 1H), 9.62 (s, 1H)

Example 369: N-(7-ethoxy-2-((tetrahydrofuran-3-yl)methyl)imidazo[1,2-a]pyridin-6-yl)-6,7-dihydro-5H-cyclopenta[b]pyridine-2-carboxamide; LCMS (ESI) m/z 407.2 (M+H)⁺; ¹H NMR (400 MHz, CHLOROFORM-d) δ ppm: 1.54-1.61 (m, 3H), 1.63-1.73 (m, 1H), 1.98-2.10 (m, 1H), 2.14-2.24 (m, 2H), 2.65-2.81 (m, 3H), 2.96-3.07 (m, 4H), 3.45-3.55 (m, 1H), 3.71-3.81 (m, 1H), 3.84-3.94 (m, 2H), 4.18 (q, J=6.9 Hz, 2H), 6.83 (s, 1H), 7.19 (s, 1H), 7.66 (d, J=8.0 Hz, 1H), 7.99 (d, J=8.0 Hz, 1H), 9.40 (s, 1H), 10.61 (s, 1H),

Example 370: 6-(difluoromethyl)-N-(8-methyl-2-(tetrahydro-2H-pyran-4-yl)imidazo[1,2-a]pyridin-6-yl)picolinamide 2,2,2-trifluoroacetate; LCMS (ESI) m/z 387.3 (M+H)⁺; ¹H NMR (500 MHz, DMSO-d₆) δ ppm 1.71-1.79 (m, 3H), 1.96 (br dd, J=12.82, 1.83 Hz, 3H), 2.54 (s, 4H), 3.12-3.20 (m, 1H), 3.34 (br s, 1H), 3.49 (td, J=11.60, 1.83 Hz, 1H), 3.96-4.00 (m, 2H), 7.00-7.25 (m, 1H), 7.83 (s, 1H), 8.03-8.08 (m, 1H), 8.15 (s, 1H), 8.31-8.37 (m, 2H), 9.28 (s, 1H), 10.47 (s, 1H).

Example 371: N-(7-(methoxymethyl)-2-(tetrahydro-2H-pyran-4-yl)imidazo[1,2-a]pyridin-6-yl)-6-methylpicolinamide 2,2,2-trifluoroacetate; LCMS (ESI) m/z 381.3 (M+H)⁺; ¹H NMR (500 MHz, DMSO-d₆) δ ppm 1.67-1.80 (m, 2H), 1.95-2.05 (m, 2H), 2.65 (s, 3H), 3.09-3.21 (m, 1H), 3.45-3.52 (m, 1H), 3.60 (s, 2H), 3.95-3.99 (m, 2H), 4.81 (s, 2H), 7.63 (dd, J=7.02, 1.53 Hz, 1H), 7.93 (s, 1H), 8.00-8.06 (m, 2H), 8.30 (s, 1H), 9.73 (s, 1H), 11.20 (s, 1H).

Example 372: 6-(difluoromethyl)-N-(7-ethoxy-2-(tetrahydro-2H-pyran-4-yl)imidazo[1,2-a]pyrimidin-6-yl)picolinamide; LCMS (ESI) m/z 418 (M+H)⁺; ¹H NMR (400 MHz, METHANOL-d₄) δ ppm 1.55 (t, J=7.0 Hz, 3H), 1.72-1.85 (m, 2H), 1.94-2.03 (m, 2H), 2.88-2.98 (m, 1H), 3.52-3.62 (m, 2H), 3.98-4.06 (m, 2H), 4.64 (q, J=7.0 Hz, 2H), 6.85 (t, J=55.0 Hz, 1H), 7.42 (s, 1H), 7.94 (d, J=7.8 Hz 1H), 8.24 (t, J=7.8 Hz, 1H), 8.36 (d, J=8.0 Hz, 1H), 9.61 (s, 1H).

Example 373: N-(7-ethoxy-2-((tetrahydrofuran-3-yl)methyl)imidazo[1,2-a]pyridin-6-yl)-1-methyl-2-oxo-1,2-dihydropyridine-3-carboxamide; LCMS (ESI) m/z 397.0 (M+H)⁺; ¹H NMR (400 MHz, METHANOL-d₄) δ ppm: 1.58 (t, J=6.9 Hz, 3H), 1.62-1.74 (m, 1H), 2.02-2.13 (m, 1H), 2.57-2.81 (m, 3H), 3.42-3.57 (m, 1H), 3.68 (s, 3H), 3.76 (q, J=7.6 Hz, 1H), 3.88 (dt, J=14.1, 7.7 Hz, 2H), 4.25 (q, J=7.0 Hz, 2H), 6.55 (t, J=7.0 Hz, 1H), 6.83 (s, 1H), 7.44 (s, 1H), 7.96 (dd, J=6.4, 2.2 Hz, 1H), 8.51 (dd, J=7.4, 2.2 Hz, 1H), 9.46 (s, 1H).

Example 374: N-(2-(3-oxabicyclo[3.1.0]hexan-6-yl)-7-ethoxyimidazo[1,2-a]pyridin-6-yl)-1-(2,2-difluorocyclopropyl)-1H-pyrazole-3-carboxamide; LCMS (ESI) m/z 430.0 (M+H)⁺;

Example 375: N-(2-(3-oxabicyclo[3.1.0]hexan-6-yl)-7-ethoxyimidazo[1,2-a]pyridin-6-yl)-2-cyclopropyloxazole-4-carboxamide; LCMS (ESI) m/z 395.0 (M+H)⁺;

Example 376: N-(2-(3-oxabicyclo[3.1.0]hexan-6-yl)-7-ethoxyimidazo[1,2-a]pyridin-6-yl)-2-(difluoromethyl)oxazole-4-carboxamide 2,2,2-trifluoroacetate; LCMS (ESI) m/z 405.1 (M+H)⁺; ¹H NMR (500 MHz, DMSO-d₆) δ ppm 1.47 (t, J=7.02 Hz, 4H), 1.96 (s, 1H), 2.19 (br d, J=3.66 Hz, 2H), 3.72 (d, J=8.55 Hz, 2H), 3.94 (d, J=8.55 Hz, 2H), 4.39 (d, J=7.32 Hz, 2H), 7.20-7.52 (m, 2H), 8.01 (s, 1H), 9.10-9.28 (m, 1H), 9.41 (s, 1H), 9.60 (s, 1H)

Example 377: N-(2-(3-oxabicyclo[3.1.0]hexan-6-yl)-7-ethoxyimidazo[1,2-a]pyridin-6-yl)-2-(trifluoromethyl)thiazole-4-carboxamide 2,2,2-trifluoroacetate; LCMS (ESI) m/z 438.9 (M+H)⁺;

Example 378: 3-(difluoromethyl)-2-fluoro-N-(7-isopropoxy-2-(tetrahydro-2H-pyran-4-yl)imidazo[1,2-a]pyridin-6-yl)benzamide 2,2,2-trifluoroacetate; LCMS (ESI) m/z 448.3 (M+H)⁺; ¹H NMR (500 MHz, DMSO-d₆) δ ppm 1.64-1.75 (m, 3H), 1.88-1.96 (m, 2H), 3.04-3.15 (m, 1H), 3.40-3.55 (m, 1H), 3.47-3.53 (m, 1H), 3.97 (dt, J=9.77, 2.14 Hz, 2H), 5.04 (dt, J=12.06, 5.88 Hz, 1H), 7.20-7.45 (m, 2H), 7.55 (t, J=7.63 Hz, 1H), 7.90 (br t, J=7.02 Hz, 1H), 7.99 (br t, J=7.02 Hz, 1H), 8.04 (s, 1H), 9.51 (s, 1H), 10.01 (br d, J=4.27 Hz, 1H).

Example 379: N-(7-isopropoxy-2-(tetrahydro-2H-pyran-4-yl)imidazo[1,2-a]pyridin-6-yl)-2,3-dihydrobenzofuran-7-carboxamide; LCMS (ESI) m/z 422.1 (M+H)⁺; ¹H NMR (400 MHz, CHLOROFORM-d) δ ppm 1.47 (d, J=6.1 Hz, 6H), 1.74-1.90 (m, 2H), 2.01 (d, J=13.4 Hz, 2H), 2.86-2.99 (m, 1H), 3.32 (t, J=8.7 Hz, 2H), 3.54 (t, J=11.7 Hz, 2H), 4.05 (d, J=11.9 Hz, 2H), 4.54-4.94 (m, 3H), 6.84 (s, 1H), 7.00 (t, J=8.0 Hz, 1H), 7.15 (s, 1H), 7.35 (d, J=7.3 Hz, 1H), 7.95 (d, J=7.9 Hz, 1H), 9.52 (s, 1H), 10.15 (s, 1H)

Example 380: N-(7-isopropoxy-2-(tetrahydro-2H-pyran-4-yl)imidazo[1,2-a]pyridin-6-yl)-6-(tetrahydrofuran-3-yl)picolinamide hydrochloride; LCMS (ESI) m/z 451.1 (M+H)⁺; ¹H NMR: (400 MHz, METHANOL-d₄) δ: 1.60 (d, J=5.87 Hz, 6H), 1.85 (qd, J=12.19, 4.11 Hz, 2H), 2.05 (d, J=12.13 Hz, 2H), 2.30-2.40 (m, 1H), 2.40-2.50 (m, 1H), 3.17 (t, J=11.93 Hz, 1H), 3.62 (t, J=11.15 Hz, 2H), 3.78 (q, 7.83 Hz, 1H), 3.98 (q, J=7.83 Hz, 1H), 4.03-4.10 (m, 3H), 4.10-4.14 (m, 1H), 4.25 (t, J=8.02 Hz, 1H), 5.11 (t, J=11.74, 5.87 Hz, 1H), 7.34 (s, 1H), 7.63 (d, J=7.43 Hz, 1H), 7.89 (s, 1H), 8.01 (t, J=7.83 Hz, 1H), 8.12 (d, J=7.43 Hz, 1H), 9.83 (s, 1H),

Example 381: N-(7-isopropoxy-2-(tetrahydro-2H-pyran-4-yl)imidazo[1,2-a]pyridin-6-yl)pyrazolo[1,5-a]pyridine-2-carboxamide 2,2,2-trifluoroacetate; LCMS (ESI) m/z 442.9 (M+Na)⁺; ¹H NMR (500 MHz, DMSO-d₆) δ ppm 1.49 (d, J=6.10 Hz, 6H), 1.71 (br dd, J=12.21, 3.66 Hz, 2H), 1.96 (br dd, J=12.51, 2.14 Hz, 2H), 3.12 (s, 1H), 3.42-3.59 (m, 1H), 3.97 (dt, J=9.77, 2.14 Hz, 2H), 5.11 (dt, J=12.21, 6.10 Hz, 1H), 7.15 (td, J=6.87, 1.53 Hz, 1H), 7.23 (s, 1H), 7.34 (s, 1H), 7.36-7.44 (m, 1H), 7.87 (d, J=9.16 Hz, 1H), 8.12 (s, 1H), 8.83 (d, J=7.94 Hz, 1H), 9.66 (s, 1H), 9.71 (s, 1H).

Example 382: N-(7-isopropoxy-2-(tetrahydro-2H-pyran-4-yl)imidazo[1,2-a]pyridin-6-yl)pyrazolo[1,5-a]pyrimidine-2-carboxamide 2,2,2-trifluoroacetate; LCMS (ESI) m/z 421.0 (M+H)⁺; ¹H NMR (500 MHz, METHANOL-d₄) δ ppm 1.58 (d, J=6.10 Hz, 6H), 1.85 (br dd, J=12.82, 3.66 Hz, 3H), 2.03 (br dd, J=12.82, 1.83 Hz, 2H), 3.09-3.19 (m, 1H), 3.61 (td, J=11.60, 1.83 Hz, 3H), 3.98-4.16 (m, 3H), 5.07 (dt, J=12.21, 6.10 Hz, 1H), 7.20 (dd, J=7.32, 4.27 Hz, 1H), 7.27 (d, J=6.10 Hz, 2H), 7.88 (s, 1H), 8.59-8.70 (m, 1H), 9.06 (s, 1H), 9.74 (s, 1H)

Example 383: N-(7-isopropoxy-2-(tetrahydro-2H-pyran-4-yl)imidazo[1,2-a]pyridin-6-yl)-1-(2-methoxyethyl)-1H-pyrazole-3-carboxamide 2,2,2-trifluoroacetate; LCMS (ESI) m/z 428.1 (M+H)⁺; ¹H NMR (500 MHz, DMSO-d₆) δ ppm 1.46 (d, J=6.10 Hz, 6H), 1.70 (br dd, J=12.51, 3.97 Hz, 2H), 1.95 (br dd, J=12.51, 2.14 Hz, 2H), 3.11 (br s, 1H), 3.49 (br d, J=1.83 Hz, 2H), 3.79 (t, J=5.19 Hz, 2H), 3.97 (br dd, J=11.90, 2.14 Hz, 2H), 4.41 (t, J=5.19 Hz, 2H), 5.09 (s, 1H), 6.85 (d, J=2.44 Hz, 1H), 7.32 (s, 1H), 7.97 (d, J=2.44 Hz, 1H), 8.11 (s, 1H), 9.44 (s, 1H), 9.60 (s, 1H)

Example 384: N-(7-isopropoxy-2-(tetrahydro-2H-pyran-4-yl)imidazo[1,2-a]pyridin-6-yl)-1-methyl-1H-pyrazole-3-carboxamide; LCMS (ESI) m/z 384.0 (M+H)⁺; ¹H NMR (400 MHz, METHANOL-d₄) δ ppm: 1.56 (d, J=6.0 Hz, 6H), 1.80-1.90 (m, 2H), 2.10-2.00 (m, 2H), 3.10-3.20 (m, 1H), 3.60-3.70 (m, 2H), 4.01 (s, 3H), 4.10-4.20 (m, 2H), 5.00-5.10 (m, 1H), 6.85 (s, 1H), 7.26 (s, 1H), 7.73 (d, J=2.0 Hz, 1H), 7.86 (s, 1H), 9.67 (s, 1H)

Example 385: 1-(difluoromethyl)-N-(7-isopropoxy-2-(tetrahydro-2H-pyran-4-yl)imidazo[1,2-a]pyridin-6-yl)-1H-pyrazole-3-carboxamide; LCMS (ESI) m/z 420.0 (M+H); ¹H NMR (400 MHz, METHANOL-d₄) δ: 1.53 (d, J=6.11 Hz, 6H), 1.77-1.88 (m, 2H), 2.01 (dd, J=12.84 Hz, 1.83 Hz, 2H), 3.17-3.09 (m, 1H), 3.59 (td, J=11.74 Hz, 1.71 Hz, 2H), 4.04 (dd, J=11.62 Hz, 2.57 Hz, 2H), 5.04 (dt, J=12.10 Hz, 5.93 Hz, 1H), 7.05 (d, J=2.69 Hz, 1H), 7.27 (s, 1H), 7.50-7.61 (m, 1H), 7.85 (s, 1H), 8.24 (d, J=2.69 Hz, 1H), 9.64 (s, 1H)

Example 386: N-(7-isopropoxy-2-(tetrahydro-2H-pyran-4-yl)imidazo[1,2-a]pyridin-6-yl)-2-methyloxazole-4-carboxamide; LCMS (ESI) m/z 358.0 (M+H)⁺; ¹H NMR (500 MHz, METHANOL-d₄) δ: 1.55 (d, J=5.95 Hz, 6H), 1.80-1.90 (m, 2H), 2.04 (d, J=11.75 Hz, 2H), 2.54 (s, 3H), 3.10-3.20 (m, 1H), 3.55-3.65 (m, 2H), 4.06 (dd, J=11.52, 2.98 Hz, 2H), 5.03-5.10 (m, 1H), 7.31 (s, 1H), 7.86 (s, 1H), 8.47 (s, 1H), 9.66 (s, 1H)

Example 387: N-(7-isopropoxy-2-(tetrahydro-2H-pyran-4-yl)imidazo[1,2-a]pyridin-6-yl)-2-(tetrahydro-2H-pyran-4-yl)oxazole-4-carboxamide 2,2,2-trifluoroacetate; LCMS (ESI) m/z 455.1 (M+H)⁺; ¹H NMR (500 MHz, DMSO-d₆) δ ppm: 1.46 (d, J=5.49 Hz, 6H), 1.62-1.85 (m, 4H), 1.88-2.06 (m, 4H), 3.11 (tt, J=11.67, 3.89 Hz, 1H), 3.25 (tt, J=10.83, 3.81 Hz, 1H), 3.44-3.59 (m, 3H), 3.82-4.04 (m, 4H), 5.04-5.12 (m, 1H), 7.34 (s, 1H), 8.13 (s, 1H), 8.90 (s, 1H), 9.53 (s, 1H), 9.60 (s, 1H).

Example 388: 3,3-difluoro-N-(7-isopropoxy-2-(1-methyl-2-oxabicyclo[2.1.1]hexan-4-yl)imidazo[1,2-a]pyridin-6-yl)-2,3-dihydrobenzofuran-7-carboxamide; LCMS (ESI) m/z 470.0 (M+H)⁺; ¹H NMR (500 MHz, METHANOL-d₄) δ ppm: 1.51 (s, 3H), 1.54 (d, J=6.0 Hz, 6H), 1.80-1.90 (m, 2H), 2.10-2.20 (m, 2H), 4.01 (s, 2H), 5.05 (t, J=15.5 Hz, 2H), 6.92 (s, 1H), 7.30-7.40 (m, 1H), 7.60 (s, 1H), 7.80-7.90 (m, 1H), 8.30-8.40 (m, 1H), 9.57 (s, 1H)

Example 389: N-(7-(difluoromethyl)-2-(tetrahydro-2H-pyran-4-yl)imidazo[1,2-a]pyridin-6-yl)-6-methoxypicolinamide 2,2,2-trifluoroacetate; LCMS (ESI) m/z 403.2 (M+H)⁺; ¹H NMR (500 MHz, DMSO-d₆) δ ppm 1.68-1.79 (m, 2H), 1.96 (br d, J=12.82 Hz, 2H), 3.11 (br t, J=11.29 Hz, 1H), 3.49 (br t, J=10.99 Hz, 1H), 3.96 (br dd, J=11.60, 1.83 Hz, 2H), 4.02 (s, 3H), 7.19 (d, J=8.55 Hz, 1H), 7.27-7.51 (m, 1H), 7.81 (d, J=7.32 Hz, 1H), 8.02 (dd, J=15.87, 8.55 Hz, 2H), 8.21 (br s, 1H), 9.57 (s, 1H), 10.49 (s, 1H).

Example 390: N-(7-(difluoromethyl)-2-(1-methyl-2-oxabicyclo[2.1.1]hexan-4-yl)imidazo[1,2-a]pyridin-6-yl)-1-methyl-1H-pyrazole-3-carboxamide 2,2,2-trifluoroacetate; LCMS (ESI) m/z 388.2 (M+H); ¹H NMR (500 MHz, DMSO-d₆) δ ppm 1.43-1.47 (m, 3H), 1.83 (dd, J=4.27, 1.22 Hz, 2H), 2.04-2.10 (m, 2H), 3.93 (s, 2H), 3.98 (s, 2H), 6.79 (d, J=2.44 Hz, 1H), 7.07-7.32 (m, 1H), 7.81-7.93 (m, 1H), 8.12 (s, 1H), 8.98 (s, 1H), 9.88 (s, 1H)

Example 391: N-(7-(difluoromethyl)-2-(1-methyl-2-oxabicyclo[2.1.1]hexan-4-yl)imidazo[1,2-a]pyridin-6-yl)-2-methoxynicotinamide 2,2,2-trifluoroacetate; LCMS (ESI) m/z 415.2 (M+H)⁺; ¹H NMR (500 MHz, DMSO-d₆) δ ppm: 1.45 (s, 3H), 1.78-1.87 (m, 2H), 2.08 (dd, J=4.58, 1.53 Hz, 2H), 3.94 (s, 2H), 4.09 (s, 3H), 7.17-7.42 (m, 2H), 7.95 (s, 1H), 8.18 (s, 1H), 8.38 (dd, J=7.63, 2.14 Hz, 1H), 8.44 (dd, J=4.58, 2.14 Hz, 1H), 9.34-9.43 (m, 1H), 9.39 (s, 1H), 10.29 (s, 1H)

Example 392: N-(7-(difluoromethyl)-2-(1-methyl-2-oxabicyclo[2.1.1]hexan-4-yl)imidazo[1,2-a]pyridin-6-yl)-6-methylpicolinamide 2,2,2-trifluoroacetate; LCMS (ESI) m/z 399.2 (M+H)⁺; ¹H NMR (500 MHz, DMSO-d₆) δ ppm 1.45 (s, 3H), 1.83 (dd, J=3.97, 1.53 Hz, 2H), 2.08 (dd, J=4.27, 1.22 Hz, 2H), 2.62 (s, 2H), 2.64 (br s, 1H), 3.94 (s, 2H), 7.14-7.43 (m, 1H), 7.55-7.63 (m, 1H), 7.95 (s, 1H), 8.00 (d, J=4.88 Hz, 1H), 8.18 (s, 1H), 9.35 (s, 1H), 10.63 (s, 1H).

Example 393: 6-(difluoromethyl)-N-(7-(difluoromethyl)-2-(1-methyl-2-oxabicyclo[2.1.1]hexan-4-yl)imidazo[1,2-a]pyridin-6-yl)picolinamide 2,2,2-trifluoroacetate; LCMS (ESI) m/z 435.1 (M+H)⁺; ¹H NMR (500 MHz, DMSO-d₆) δ ppm: 1.45 (s, 3H), 1.79-1.87 (m, 2H), 2.08 (dd, J=4.27, 1.83 Hz, 2H), 3.94 (s, 2H), 6.97-7.39 (m, 2H) 7.94 (s, 1H), 8.04 (dd, J=6.41, 2.14 Hz, 1H), 8.16 (s, 1H), 8.28-8.38 (m, 2H), 9.23 (s, 1H), 10.54 (s, 1H)

Example 394: N-(7-(difluoromethoxy)-2-(tetrahydro-2H-pyran-4-yl)imidazo[1,2-a]pyridin-6-yl)-6-(difluoromethyl)picolinamide; LCMS (ESI) m/z 439.2 (M+H)⁺; ¹H NMR (500 MHz, DMSO-d₆) δ ppm 1.67-1.78 (m, 3H), 1.95 (br dd, J=12.82, 1.83 Hz, 3H), 3.12 (br t, J=11.60 Hz, 1H), 3.46-3.52 (m, 2H), 3.92-3.99 (m, 2H), 7.00-7.25 (m, 1H), 7.47-7.77 (m, 2H), 8.06 (dd, J=7.32, 1.83 Hz, 1H), 8.15 (br s, 1H), 8.31-8.39 (m, 2H), 9.64 (br s, 1H), 10.44 (s, 1H).

Example 395: N-(7-(difluoromethoxy)-2-(1-methyl-2-oxabicyclo[2.1.1]hexan-4-yl)imidazo[1,2-a]pyridin-6-yl)-6-(difluoromethyl)picolinamide 2,2,2-trifluoroacetate; LCMS (ESI) m/z 451.2 (M+H)⁺; ¹H NMR (500 MHz, DMSO-d₆) δ ppm 1.45 (s, 4H), 1.75-1.87 (m, 2H), 2.05-2.13 (m, 2H), 3.93 (s, 2H), 6.99-7.26 (m, 1H), 7.38-7.71 (m, 2H), 8.05 (dd, J=6.71, 1.83 Hz, 1H), 8.16 (br s, 1H), 8.30-8.40 (m, 2H), 9.56 (br s, 1H), 10.41 (s, 1H)

Example 396: N-(7-(difluoromethoxy)-2-(1-methyl-2-oxabicyclo[2.1.1]hexan-4-yl)imidazo[1,2-a]pyridin-6-yl)-2-methoxynicotinamide 2,2,2-trifluoroacetate; LCMS (ESI) m/z 431.3 (M+H)⁺; ¹H NMR (500 MHz, DMSO-d₆) δ ppm 1.45 (s, 3H), 1.79-1.88 (m, 2H), 2.08 (br d, J=3.05 Hz, 2H), 3.92 (s, 2H), 4.15 (s, 3H), 7.31 (dd, J=7.32, 4.88 Hz, 1H), 7.45-7.66 (m, 2H), 8.16 (br s, 1H), 8.40-8.52 (m, 1H), 9.74 (br s, 1H), 10.52 (br s, 1H)

Example 397: N-(7-(difluoromethoxy)-2-(1-methyl-2-oxabicyclo[2.1.1]hexan-4-yl)imidazo[1,2-a]pyridin-6-yl)-1-methyl-1H-pyrazole-3-carboxamide 2,2,2-trifluoroacetate; LCMS (ESI) m/z 404.2 (M+H); ¹H NMR (500 MHz, DMSO-d₆) δ ppm 1.44 (s, 3H), 1.75-1.83 (m, 2H), 2.04 (br d, J=3.66 Hz, 2H), 3.90 (s, 2H), 3.98 (s, 3H), 6.80 (d, J=1.83 Hz, 1H), 7.25-7.58 (m, 2H), 7.91 (d, J=1.83 Hz, 1H), 7.98 (br s, 1H), 9.18 (br s, 1H), 9.45 (br s, 1H)

Example 398: 6-(difluoromethyl)-N-(2-(1-methyl-2-oxabicyclo[2.1.1]hexan-4-yl)-7-((1,1,1-trifluoropropan-2-yl)oxy)imidazo[1,2-a]pyridin-6-yl)picolinamide 2,2,2-trifluoroacetate; LCMS (ESI) m/z 497.2 (M+H); ¹H NMR (500 MHz, DMSO-d₆) δ ppm 1.46 (s, 3H), 1.62 (d, J=6.10 Hz, 3H), 1.86 (br d, J=3.05 Hz, 2H), 2.13 (br s, 2H), 3.94 (s, 2H), 5.85 (br s, 1H), 6.95-7.21 (m, 1H), 7.62 (br s, 1H), 8.06 (dd, J=6.71, 1.83 Hz, 1H), 8.22 (br s, 1H), 8.32-8.40 (m, 2H), 9.75 (br s, 1H), 10.55 (br s, 1H)

Example 399: 1-methyl-N-(2-(1-methyl-2-oxabicyclo[2.1.1]hexan-4-yl)-7-((1,1,1-trifluoropropan-2-yl)oxy)imidazo[1,2-a]pyridin-6-yl)-1H-pyrazole-3-carboxamide 2,2,2-trifluoracetate; LCMS (ESI) m/z 450.3 (M+H)⁺; ¹H NMR (500 MHz, DMSO-d₆) δ ppm 1.46 (s, 3H), 1.58 (d, J=6.10 Hz, 3H), 1.81-1.91 (m, 2H), 2.14 (br d, J=3.05 Hz, 2H), 3.88-4.00 (m, 4H), 5.83 (br s, 1H), 6.83 (d, J=1.83 Hz, 1H), 7.57 (s, 1H), 7.94 (d, J=2.44 Hz, 1H), 8.20 (br s, 1H), 9.45 (br s, 1H), 9.54 (br s, 1H)

Example 400: N-(2-chloro-7-isopropoxyimidazo[1,2-a]pyridin-6-yl)-2-methoxybenzamide 2,2,2-trifluoroacetate; LCMS (ESI) m/z 360.0 (M+H)⁺; ¹H NMR (400 MHz, DMSO-d₆) δ ppm: 1.44 (d, J=6.02 Hz, 6H), 4.10 (s, 4H), 4.88-4.99 (m, 1H), 7.15 (s, 1H), 7.16-7.22 (m, 1H), 7.33 (d, J=8.03 Hz, 1H), 7.59-7.68 (m, 1H), 7.99 (s, 1H), 8.10 (dd, J=1.76, 7.78 Hz, 1H), 9.64 (s, 1H), 10.40 (s, 1H)

Example 401: N-(2-chloro-7-isopropoxyimidazo[1,2-a]pyridin-6-yl)-2-methoxynicotinamide; LCMS (ESI) m/z 361.0 (M+H)⁺; ¹H NMR (400 MHz, METHANOL-d₄) δ ppm: 1.58 (d, J=6.02 Hz, 6H), 4.27 (s, 3H), 7.12 (s, 1H), 7.20-7.25 (m, 1H), 7.92 (s, 1H), 8.41 (dd, J=2.01, 4.77 Hz, 1H), 8.57 (dd, J=2.01, 7.53 Hz, 1H), 9.76 (s, 1H)

Example 402: N-(2-chloro-7-isopropoxyimidazo[1,2-a]pyridin-6-yl)-3-fluoro-2-methoxybenzamide 2,2,2-trifluoroacetate; LCMS (ESI) m/z 378.0 (M+H)⁺; ¹H NMR (400 MHz, DMSO-d₆) δ ppm: 1.42 (d, J=6.02 Hz, 6H), 4.11 (d, J=2.01 Hz, 5H), 4.89-4.99 (m, 1H), 7.16 (s, 1H), 7.30-7.37 (m, 1H), 7.55-7.63 (m, 1H), 7.84-7.90 (m, 1H), 8.00 (s, 1H), 9.58 (s, 1H), 10.47 (s, 1H).

Example 403: 6-(difluoromethyl)-N-(7-isopropoxy-2-(3-methoxypropyl)imidazo[1,2-a]pyridin-6-yl)picolinamide hydrochloride; LCMS (ESI) m/z 419.0 (M+H); ¹H NMR (500 MHz, METHANOL-d₄) δ ppm: 1.59 (d, J=6.0 Hz, 6H), 2.00-2.06 (m, 2H), 2.90-2.94 (m, 2H), 3.37 (s, 3H), 3.50 (t, J=6.0 Hz, 2H), 5.05-5.10 (m, 1H), 6.76-6.99 (m, 1H), 7.30 (s, 1H), 7.86 (s, 1H), 7.96-7.98 (m, 1H), 8.26-8.30 (m, 1H), 8.39-8.42 (m, 1H), 9.78 (s, 1H).

Example 404: 6-(difluoromethyl)-N-(7-isobutyl-2-(tetrahydro-2H-pyran-4-yl)imidazo[1,2-a]pyridin-6-yl)picolinamide; LCMS (ESI) m/z 429.3 (M+H)⁺; ¹H NMR (500 MHz, DMSO-d₆) δ ppm 0.94 (d, J=6.71 Hz, 7H), 1.71-1.79 (m, 2H), 1.95-2.02 (m, 3H), 2.83 (d, J=6.71 Hz, 2H), 3.12-3.22 (m, 1H), 3.50 (br dd, J=11.60, 9.77 Hz, 3H), 3.93-4.00 (m, 2H), 7.01-7.25 (m, 1H), 7.77 (s, 1H), 8.05-8.08 (m, 1H), 8.21 (s, 1H), 8.34-8.38 (m, 2H), 9.45 (s, 1H), 10.46 (s, 1H)

Example 405: N-(7-chloro-2-(tetrahydro-2H-pyran-4-yl)imidazo[1,2-a]pyridin-6-yl)-6-(difluoromethyl)picolinamide 2,2,2-trifluoroacetate; LCMS (ESI) m/z 407.2 (M+H)⁺; ¹H NMR (500 MHz, DMSO-d₆) δ ppm 1.69-1.78 (m, 2H), 1.95 (br dd, J=12.82, 2.44 Hz, 2H), 3.09 (br t, J=10.99 Hz, 1H), 3.49 (td, J=11.60, 1.83 Hz, 1H), 3.96 (dt, J=9.61, 1.91 Hz, 2H), 6.98-7.30 (m, 1H), 8.07 (dd, J=6.71, 1.83 Hz, 1H), 8.12 (br s, 1H), 8.33-8.39 (m, 2H), 9.47 (br s, 1H), 10.60 (s, 1H)

Example 406: 6-(difluoromethyl)-N-(8-methyl-2-(1-methyl-2-oxabicyclo[2.1.1]hexan-4-yl)imidazo[1,2-a]pyridin-6-yl)picolinamide; LCMS (ESI) m/z 399.3 (M+H); ¹H NMR (500 MHz, CHLOROFORM-d) δ ppm: 1.55-1.60 (m, 3H) 2.01-2.08 (m, 2H) 2.30-2.35 (m, 2H) 2.75-2.79 (m, 3H) 4.12 (s, 2H) 6.63-6.89 (m, 1H) 7.50-7.55 (m, 2H) 7.95 (d, J=6.71 Hz, 1H) 8.19 (t, J=7.94 Hz, 1H) 8.45 (d, J=7.33 Hz, 1H) 9.56 (d, J=1.22 Hz, 1H) 10.00 (s, 1H)

Example 407: 3-fluoro-2-methoxy-N-(8-methyl-2-(1-methyl-2-oxabicyclo[2.1.1]hexan-4-yl)imidazo[1,2-a]pyridin-6-yl)benzamide; LCMS (ESI) m/z 396.3 (M+H)⁺; ¹H NMR (500 MHz, DMSO-d₆) δ ppm 1.44 (s, 3H), 1.77 (br s, 2H), 2.02 (br s, 2H), 3.90 (s, 2H), 3.93 (d, J=1.83 Hz, 2H), 7.10 (br s, 1H), 7.24 (td, J=7.94, 4.88 Hz, 1H), 7.39 (d, J=7.94 Hz, 1H), 7.46 (td, J=10.07, 1.22 Hz, 1H), 7.92 (br s, 1H), 9.12 (br s, 1H), 10.36 (br s, 1H)

Example 408: N-(8-methyl-2-(1-methyl-2-oxabicyclo[2.1.1]hexan-4-yl)imidazo[1,2-a]pyridin-6-yl)pyrazolo[1,5-a]pyridine-7-carboxamide 2,2,2-trifluoroacetate; LCMS (ESI) m/z 388.3 (M+H)⁺; ¹H NMR (500 MHz, DMSO-d₆) δ ppm: 1.47 (s, 3H), 1.88 (br s, 2H), 2.17 (br s, 2H), 3.97 (s, 2H), 6.98 (d, J=2.44 Hz, 1H), 7.50 (dd, J=9.16, 7.32 Hz, 1H), 7.86 (dd, J=7.02, 1.53 Hz, 1H), 8.01-8.16 (m, 1H), 8.30 (d, J=2.44 Hz, 1H), 9.55 (br s, 1H), 12.78 (br s, 1H)

Example 409: 1-methyl-N-(8-methyl-2-(1-methyl-2-oxabicyclo[2.1.1]hexan-4-yl)imidazo[1,2-a]pyridin-6-yl)-1H-pyrazole-3-carboxamide 2,2,2-trifluoroacetate; LCMS (ESI) m/z 352.2 (M+H)⁺; ¹H NMR (500 MHz, DMSO-d₆) δ ppm: 1.41-1.51 (m, 3H), 1.81-1.94 (m, 2H), 2.12-2.24 (m, 2H), 3.92-3.98 (m, 2H), 3.99 (s, 3H), 6.81 (d, J=2.44 Hz, 1H), 6.96-7.22 (m, 1H), 7.90 (d, J=2.44 Hz, 1H), 8.02 (br s, 1H), 8.37 (br s, 1H), 9.40 (br s, 1H), 10.62 (br s, 1H)

Example 410: 6-(difluoromethyl)-N-(8-methyl-2-(tetrahydro-2H-pyran-4-yl)imidazo[1,2-a]pyridin-6-yl)picolinamide 2,2,2-trifluoroacetate; LCMS (ESI) m/z 387.3 (M+H)⁺; ¹H NMR (500 MHz, DMSO-d₆) δ ppm 1.72-1.81 (m, 2H), 1.98 (br dd, J=12.82, 1.83 Hz, 2H), 2.60 (s, 3H), 3.11-3.19 (m, 1H), 3.47-3.55 (m, 2H), 3.95-4.02 (m, 2H), 6.96-7.20 (m, 2H), 7.99-8.12 (m, 2H), 8.29-8.34 (m, 2H), 9.51 (br s, 1H), 10.94 (br s, 1H)

Example 411: 6-(difluoromethyl)-N-(8-methoxy-2-((tetrahydrofuran-3-yl)methyl)imidazo[1,2-a]pyridin-6-yl)picolinamide; LCMS (ESI) m/z 403.2 (M+H)⁺; ¹H NMR (500 MHz, METHANOL-d₄) δ ppm: 1.67-1.74 (m, 1H), 2.05-2.12 (m, 1H), 2.69-2.76 (m, 1H), 2.82 (d, J=7.5 Hz, 2H), 3.30-3.53 (m, 1H), 3.75-3.80 (m, 1H), 3.85-3.92 (m, 2H), 4.05 (s, 3H), 6.79-7.03 (m, 2H), 7.66 (s, 1H), 7.95 (d, J=9.0 Hz, 1H), 8.23 (t, J=7.5 Hz, 1H), 8.36 (d, J=7.5 Hz, 1H), 8.94 (s, 1H)

Example 412: N-(8-methoxy-2-(tetrahydro-2H-pyran-4-yl)imidazo[1,2-a]pyridin-6-yl)-1-methyl-1H-pyrazole-3-carboxamide 2,2,2-trifluoroacetate; ¹H NMR (500 MHz, DMSO-d₆) δ ppm 1.67-1.78 (m, 2H) 1.95 (br dd, J=12.82, 1.83 Hz, 2H) 3.04-3.14 (m, 1H) 3.47 (td, J=11.60, 1.83 Hz, 1H) 3.92-3.98 (m, 2H) 4.00 (s, 2H) 4.07 (s, 3H) 6.82 (d, J=1.83 Hz, 1H) 7.77-7.86 (m, 1H) 7.91 (d, J=2.44 Hz, 1H) 8.29 (s, 1H) 9.27 (s, 1H) 10.56 (br s, 1H)

Example 413: 6-(difluoromethyl)-N-(8-ethoxy-2-(tetrahydro-2H-pyran-4-yl)imidazo[1,2-a]pyridin-6-yl)picolinamide; LCMS (ESI) m/z 417.1 (M+H)⁺; ¹H NMR (500 MHz, METHANOL-d₄) δ: 1.55 (t, J=7.0 Hz, 3H), 1.75-1.90 (m, 2H), 2.01 (d, J=13.0 Hz, 2H), 2.94-3.01 (m, 1H), 3.59 (t, J=11.5 Hz, 2H), 4.03 (dd, J=11.0 Hz, J=3.0 Hz, 2H), 4.27 (q, J=7.0 Hz, 2H), 7.00-6.70 (m, 2H), 7.60 (s, 1H), 7.92 (d, J=8.0 Hz, 1H), 8.20 (t, J=8.0 Hz, 1H), 8.33 (d, J=8.0 Hz, 1H), 8.90 (s, 1H)

Example 414: 6-(difluoromethyl)-N-(8-ethoxy-2-(1-methyl-2-oxabicyclo[2.1.1]hexan-4-yl)imidazo[1,2-a]pyridin-6-yl)picolinamide 2,2,2-trifluoroacetate; LCMS (ESI) m/z 429.3 (M+H)⁺; ¹H NMR (400 MHz, CHLOROFORM-d) δ ppm: 1.53-1.57 (m, 5H), 2.04 (dd, J=4.77, 1.76 Hz, 2H), 2.30 (dd, J=4.77, 1.76 Hz, 2H), 4.10 (s, 2H), 4.33 (q, J=7.03 Hz, 2H), 6.61-6.92 (m, 1H), 6.96 (d, J=1.51 Hz, 1H), 7.47 (s, 1H), 7.93 (d, J=8.03 Hz, 1H), 8.17 (t, J=7.91 Hz, 1H), 8.42 (d, J=7.78 Hz, 1H), 9.20 (d, J=1.26 Hz, 1H), 9.92 (s, 1H).

Example 415: N-(8-ethoxy-2-(tetrahydro-2H-pyran-4-yl)imidazo[1,2-a]pyridin-6-yl)-6-(trifluoromethyl)picolinamide hydrochloride; LCMS (ESI) m/z 435.0 (M+H)⁺; ¹H NMR (500 MHz, METHANOL-d₄) δ: 1.60 (t, J=7.5 Hz, 3H, 1.80-2.00 (m, 2H), 2.06 (d, J=12.5 Hz, 2H), 3.21 (t, J=12.0 Hz, 1H), 3.62 (t, J=12.0 Hz, 2H), 4.07 (dd, J=11.0 Hz, J=3.0 Hz, 2H), 4.45 (q, J=7.0 Hz, 2H), 7.77 (s, 1H), 8.06-8.14 (m, 2H), 8.33 (t, J=8.0 Hz, 1H), 8.48 (d, J=8.0 Hz, 1H), 9.32 (s, 1H)

Example 416: N-(2-((1R,5S,6s)-3-oxabicyclo[3.1.0]hexan-6-yl)-8-ethoxyimidazo[1,2-a]pyridin-6-yl)-6-(trifluoromethyl)picolinamide; LCMS (ESI) m/z 433.0 (M+H)⁺; ¹H NMR (500 MHz, METHANOL-d₄) δ: 1.57 (t, J=7.0 Hz, 3H), 1.95 (t, J=3.5 Hz, 1H), 2.12 (t, J=3.5 Hz, 2H), 3.83 (d, J=8.5 Hz, 2H), 4.02 (d, J=8.0 Hz, 2H), 4.30 (q, J=7.0 Hz, 2H), 7.00 (d, J=1.5 Hz, 1H), 7.61 (s, 1H), 8.08 (d, J=8.0 Hz, 1H), 8.32 (t, J=8.0 Hz, 1H), 8.48 (d, J=8.0 Hz, 1H), 8.89 (s, 1H)

Example 417: N-(2-((1R,5S,6s)-3-oxabicyclo[3.1.0]hexan-6-yl)-8-ethoxyimidazo[1,2-a]pyridin-6-yl)-6-methylpicolinamide; LCMS (ESI) m/z 378.4 (M+H)⁺; ¹H NMR (500 MHz, METHANOL-d₄) δ: 1.56 (t, J=7.0 Hz, 3H), 1.93 (t, J=3.5 Hz, 1H), 2.12 (br s, 2H), 2.67 (s, 3H), 3.81 (d, J=8.0 Hz, 2H), 4.00 (d, J=8.5 Hz, 2H), 4.29 (q, J=7.0 Hz, 2H), 6.96 (s, 1H), 7.49 (d, J=7.5 Hz, 1H), 7.59 (s, 1H), 7.89 (t, J=7.5 Hz, 1H), 8.01 (d, J=7.5 Hz, 1H), 8.87 (s, 1H)

Example 418: 6-(difluoromethyl)-N-(8-isopropoxy-2-(tetrahydro-2H-pyran-4-yl)imidazo[1,2-a]pyridin-6-yl)picolinamide 2,2,2-trifluoroacetate; LCMS (ESI) m/z 431.3 (M+H)⁺; ¹H NMR (500 MHz, DMSO-d₆) δ ppm 1.48 (d, J=6.10 Hz, 7H), 1.74 (qd, J=12.31, 3.97 Hz, 2H), 1.97 (br dd, J=12.51, 2.14 Hz, 2H), 3.11 (br s, 1H), 3.48 (td, J=11.60, 1.83 Hz, 1H), 3.93-4.03 (m, 2H), 4.91 (quin, J=6.10 Hz, 1H), 6.99-7.23 (m, 1H), 7.82 (br s, 1H), 8.06 (dd, J=7.02, 2.14 Hz, 1H), 8.28-8.37 (m, 2H), 9.32 (br s, 1H), 10.86 (br s, 1H).

Example 419: N-(8-(difluoromethoxy)-2-(1-methyl-2-oxabicyclo[2.1.1]hexan-4-yl)imidazo[1,2-a]pyridin-6-yl)-6-(difluoromethyl)picolinamide; LCMS (ESI) m/z 451.2 (M+H)⁺; ¹H NMR (400 MHz, CHLOROFORM-d) δ ppm: 1.56 (s, 3H), 2.02-2.08 (m, 2H), 2.32 (dd, J=4.77, 1.76 Hz, 2H), 4.11 (s, 2H), 6.64-7.08 (m, 2H), 7.57-7.60 (m, 2H), 7.92-7.97 (m, 1H), 8.17 (t, J=7.91 Hz, 1H), 8.42 (d, J=7.78 Hz, 1H), 9.56 (d, J=1.76 Hz, 1H), 10.05 (s, 1H)

Example 420: N-(8-(difluoromethoxy)-2-(1-methyl-2-oxabicyclo[2.1.1]hexan-4-yl)imidazo[1,2-a]pyridin-6-yl)-2-methoxynicotinamide 2,2,2-trifluoroacetate; LCMS (ESI) m/z 431.2 (M+H)⁺; ¹H NMR (500 MHz, DMSO-d₆) δ ppm 1.44 (s, 3H), 1.73-1.84 (m, 2H), 2.05-2.12 (m, 2H), 3.92 (s, 2H), 4.00 (s, 3H), 7.18 (dd, J=7.32, 4.88 Hz, 1H), 7.37 (br s, 1H), 7.43-7.75 (m, 1H), 8.08 (dd, J=7.63, 2.14 Hz, 1H), 8.17 (br s, 1H), 8.37 (dd, J=4.88, 1.83 Hz, 1H), 9.25 (br s, 1H), 10.45 (br s, 1H).

Example 421: N-(8-(difluoromethoxy)-2-(1-methyl-2-oxabicyclo[2.1.1]hexan-4-yl)imidazo[1,2-a]pyridin-6-yl)-2,3-dihydrobenzofuran-7-carboxamide 2,2,2-trifluoroacetate; LCMS (ESI) m/z 442.1 (M+H)⁺; ¹H NMR (500 MHz, DMSO-d₆) δ ppm 1.44 (s, 3H), 1.80 (dd, J=4.27, 1.22 Hz, 2H), 2.03-2.09 (m, 2H), 3.29 (t, J=8.85 Hz, 1H), 3.51-3.60 (m, 3H), 3.92 (s, 2H), 4.75 (t, J=8.55 Hz, 2H), 6.97-7.04 (m, 1H), 7.39 (br s, 1H), 7.44-7.51 (m, 1H), 7.53-7.62 (m, 1H), 8.13 (br s, 1H), 9.23 (br s, 1H), 9.95 (br s, 1H).

Example 422: N-(8-(difluoromethoxy)-2-(1-methyl-2-oxabicyclo[2.1.1]hexan-4-yl)imidazo[1,2-a]pyridin-6-yl)pyrazolo[1,5-a]pyridine-7-carboxamide 2,2,2-trifluoroacetate; LCMS (ESI) m/z 440.1 (M+H)⁺; ¹H NMR (500 MHz, DMSO-d₆) δ ppm 1.45 (s, 3H), 1.76-1.85 (m, 2H), 2.04-2.11 (m, 2H), 3.93 (s, 2H), 6.96 (d, J=2.44 Hz, 1H), 7.40 (br s, 1H), 7.44-7.54 (m, 1H), 7.67 (s, 1H), 7.77-7.84 (m, 1H), 8.01-8.09 (m, 1H), 8.17 (s, 1H), 8.29 (d, J=2.44 Hz, 1H), 9.32 (s, 1H), 12.51 (s, 1H).

Example 423: N-(8-(difluoromethoxy)-2-(1-methyl-2-oxabicyclo[2.1.1]hexan-4-yl)imidazo[1,2-a]pyridin-6-yl)-1-methyl-1H-pyrazole-3-carboxamide 2,2,2-trifluoroacetate; LCMS (ESI) m/z 404.2 (M+H); ¹H NMR (500 MHz, DMSO-d₆) δ ppm 3.89 (s, 3H), 4.08 (dd, J=11.29, 3.97 Hz, 1H), 4.37 (dd, J=10.07, 3.97 Hz, 1H), 4.52 (dd, J=11.29, 7.02 Hz, 1H), 4.79-4.86 (m, 1H), 5.59-5.67 (m, 1H), 5.71 (dd, J=10.38, 1.83 Hz, 1H), 6.14 (dd, J=17.09, 1.83 Hz, 1H), 6.38 (dd, J=17.09, 10.38 Hz, 1H), 6.89-6.94 (m, 1H), 8.00-8.08 (m, 1H), 8.24 (s, 1H), 8.76-8.83 (m, 1H)

Example 424: N-(8-fluoro-7-isopropoxy-2-(1-methyl-2-oxabicyclo[2.1.1]hexan-4-yl)imidazo[1,2-a]pyridin-6-yl)-2-methoxybenzamide 2,2,2-trifluoroacetate; LCMS (ESI) m/z 440.0 (M+H); ¹H NMR (500 MHz, DMSO-d₆) δ ppm: 1.35-1.51 (m, 10H), 1.82 (br d, J=3.20 Hz, 2H), 2.08 (br s, 2H), 3.92 (s, 4H), 4.12 (s, 4H), 4.92 (br s, 1H), 7.20 (t, J=7.25 Hz, 1H), 7.36 (d, J=8.09 Hz, 1H), 7.58-7.71 (m, 1H), 8.06-8.18 (m, 2H), 9.58 (br s, 1H), 10.43 (s, 1H)

Example 425: 2-fluoro-N-(8-fluoro-7-isopropoxy-2-(1-methyl-2-oxabicyclo[2.1.1]hexan-4-yl)imidazo[1,2-a]pyridin-6-yl)-3-methylbenzamide 2,2,2-trifluoroacetate; LCMS (ESI) m/z 442.0 (M+H); ¹H NMR (500 MHz, METHANOL-d₄) δ ppm: 1.52 (s, 3H), 1.54 (d, J=5.49 Hz, 6H), 1.97 (dd, J=1.83, 4.58 Hz, 2H), 2.20 (dd, J=1.68, 4.58 Hz, 2H), 2.40 (d, J=2.29 Hz, 3H), 4.05 (s, 2H), 5.14-5.24 (m, 1H), 7.29 (t, J=7.71 Hz, 1H), 7.51-7.61 (m, 1H), 7.85-7.95 (m, 1H), 8.02 (d, J=2.44 Hz, 1H), 9.58 (s, 1H)

Example 426: N-(8-fluoro-7-isopropoxy-2-(1-methyl-2-oxabicyclo[2.1.1]hexan-4-yl)imidazo[1,2-a]pyridin-6-yl)-6-methoxypicolinamide; LCMS (ESI) m/z 441.2 (M+H)⁺; ¹H NMR (500 MHz, DMSO-d₆) δ ppm: 1.38 (d, J=6.10 Hz, 6H), 1.43 (s, 3H), 1.76 (dd, J=1.60, 4.35 Hz, 2H), 2.01 (dd, J=1.45, 4.20 Hz, 2H), 3.89 (s, 2H), 4.07 (s, 3H), 4.71-4.86 (m, 1H), 7.18 (dd, J=0.76, 8.24 Hz, 1H), 7.82 (dd, J=0.69, 7.25 Hz, 1H), 7.94-8.06 (m, 2H), 9.39 (s, 1H), 10.19 (s, 1H)

Example 427: 6-(difluoromethyl)-N-(7-methoxy-8-methyl-2-(tetrahydro-2H-pyran-4-yl)imidazo[1,2-a]pyridin-6-yl)picolinamide; LCMS (ESI) m/z 417.3 (M+H)⁺; ¹H NMR (400 MHz, CHLOROFORM-d) δ ppm 1.72-1.90 (m, 2H), 2.07-2.16 (m, 2H), 2.66 (s, 3H), 3.25 (tt, J=12.02, 3.80 Hz, 1H), 3.53-3.66 (m, 2H), 4.05-4.14 (m, 5H), 6.58-6.91 (m, 1H), 7.36 (s, 1H), 7.92 (d, J=7.78 Hz, 1H), 8.17 (t, J=7.78 Hz, 1H), 8.42 (d, J=7.78 Hz, 1H), 9.64 (s, 1H), 10.58 (s, 1H).

Example 428: N-(8-fluoro-7-isopropoxy-2-(1-methyl-2-oxabicyclo[2.1.1]hexan-4-yl)imidazo[1,2-a]pyridin-6-yl)-2-methoxynicotinamide; LCMS (ESI) m/z 441.0 (M+H)⁺; ¹H NMR (500 MHz, DMSO-d₆) δ ppm: 1.40 (d, J=6.10 Hz, 6H), 1.43 (s, 3H), 1.73-1.79 (m, 2H), 1.97-2.04 (m, 2H), 3.89 (s, 2H), 3.97 (s, 1H), 4.74-4.83 (m, 1H), 7.26-7.33 (m, 1H), 7.98 (d, J=3.05 Hz, 1H), 8.46 (d, J=6.10 Hz, 2H), 9.42 (s, 1H), 10.27 (s, 1H)

Example 429: 6-(2,2-difluoroethoxy)-N-(8-fluoro-7-isopropoxy-2-(1-methyl-2-oxabicyclo[2.1.1]hexan-4-yl)imidazo[1,2-a]pyridin-6-yl)picolinamide 2,2,2-trifluoroacetate; LCMS (ESI) m/z 490.9 (M+H)⁺; ¹H NMR (500 MHz, DMSO-d₆) δ ppm: 1.41 (d, J=5.49 Hz, 6H), 1.45 (s, 3H), 1.81 (dd, J=3.97, 1.53 Hz, 2H), 2.07 (br d, J=3.66 Hz, 2H), 3.92 (s, 2H), 4.78 (td, J=14.50, 3.97 Hz, 2H), 4.89 (br s, 1H), 6.29-6.68 (m, 1H), 7.30 (d, J=8.55 Hz, 1H), 7.90 (d, J=7.32 Hz, 1H), 7.99-8.17 (m, 2H), 9.44 (br s, 1H), 10.14 (s, 1H)

Example 430: 2-(difluoromethyl)-N-(8-fluoro-7-isopropoxy-2-(1-methyl-2-oxabicyclo[2.1.1]hexan-4-yl)imidazo[1,2-a]pyridin-6-yl)oxazole-4-carboxamide 2,2,2-trifluoroacetate; LCMS (ESI) m/z 451.0 (M+H)⁺; ¹H NMR (500 MHz, METHANOL-d₄) δ ppm: 1.52 (s, 3H), 1.56 (dd, J=0.76, 6.10 Hz, 6H), 1.99 (dd, J=1.68, 4.58 Hz, 2H), 2.22 (dd, J=1.60, 4.65 Hz, 2H), 3.35 (s, 6H), 4.05 (s, 2H), 5.12-5.24 (m, 1H), 6.90-7.18 (m, 1H), 8.08 (d, J=2.29 Hz, 1H), 8.84 (s, 1H), 9.56 (d, J=0.92 Hz, 1H)

Example 431: 2-cyclopropyl-N-(8-fluoro-7-isopropoxy-2-(1-methyl-2-oxabicyclo[2.1.1]hexan-4-yl)imidazo[1,2-a]pyridin-6-yl)oxazole-4-carboxamide; LCMS (ESI) m/z 441.0 (M+H)⁺; 1H NMR (500 MHz, METHANOL-d₄) δ 1.07-1.14 (m, 2H), 1.15-1.23 (m, 2H), 1.55 (dd, J=0.76, 6.10 Hz, 7H), 1.52 (s, 4H), 1.91-2.03 (m, 2H), 2.12-2.25 (m, 3H), 4.04 (s, 2H), 5.05-5.19 (m, 1H), 8.02 (d, J=2.44 Hz, 1H), 8.42 (s, 1H), 9.51 (s, 1H)

Example 432: N-(8-fluoro-7-isopropoxy-2-(1-methyl-2-oxabicyclo[2.1.1]hexan-4-yl)imidazo[1,2-a]pyridin-6-yl)-1-methyl-1H-pyrazole-3-carboxamide 2,2,2-trifluoroacetate; LCMS (ESI) m/z 414.0 (M+H)⁺; ¹H NMR (500 MHz, METHANOL-d₄) δ ppm: 1.52 (s, 3H), 1.55 (dd, J=0.69, 6.03 Hz, 6H), 1.98 (dd, J=1.75, 4.65 Hz, 2H), 2.21 (dd, J=1.68, 4.58 Hz, 2H), 3.98-4.08 (m, 5H), 5.06-5.18 (m, 1H), 6.86 (d, J=2.29 Hz, 1H), 7.74 (d, J=2.29 Hz, 1H), 8.04 (d, J=2.44 Hz, 1H), 9.52 (s, 1H)

Example 433: N-(8-fluoro-7-isopropoxy-2-(1-methyl-2-oxabicyclo[2.1.1]hexan-4-yl)imidazo[1,2-a]pyridin-6-yl)pyrazolo[1,5-a]pyrimidine-7-carboxamide; LCMS (ESI) m/z 451.1 (M+H)⁺; ¹H NMR (500 MHz, CHLOROFORM-d₄) δ ppm: 1.53 (s, 3H), 1.54 (d, J=6.0 Hz, 6H), 1.97-1.99 (m, 2H), 2.11-2.13 (m, 2H), 4.10 (s, 2H), 5.07-5.10 (m, 1H), 6.99 (d, J=2.5 Hz, 1H), 7.41 (d, J=3.0 Hz, 1H), 7.91 (d, J=4.5 Hz, 1H), 8.32 (d, J=2.5 Hz, 1H), 8.77 (d, J=4.5 Hz, 1H), 9.48 (s, 1H), 13.11 (s, 1H).

Example 434: N-(8-fluoro-7-isopropoxy-2-(1-methyl-2-oxabicyclo[2.1.1]hexan-4-yl)imidazo[1,2-a]pyridin-6-yl)-[1,2,4]triazolo[1,5-a]pyridine-5-carboxamide; LCMS (ESI) m/z 451.1 (M+H); ¹H NMR (500 MHz, METHANOL-d₄) δ ppm: 1.49 (s, 3H), 1.51-1.54 (m, 6H), 1.88-1.92 (m, 2H), 2.12-2.15 (m, 2H), 4.03 (s, 2H), 5.01-5.07 (m, 1H), 7.74 (s, 1H), 7.90-7.93 (m, 1H), 8.09 (d, J=8.0 Hz, 1H), 8.23 (d, J=6.5 Hz, 1H), 8.76 (s, 1H), 9.54 (s, 1H).

Example 435: N-(8-fluoro-7-isopropoxy-2-(1-methyl-2-oxabicyclo[2.2.1]heptan-4-yl)imidazo[1,2-a]pyridin-6-yl)-2-methoxynicotinamide 2,2,2-trifluoroacetate; LCMS (ESI) m/z 455.0 (M+H); ¹H NMR (500 MHz, METHANOL-d₄) δ ppm: 1.46 (s, 4H), 1.49 (d, J=6.10 Hz, 6H), 1.76-1.92 (m, 3H), 1.96-2.02 (m, 2H), 2.05-2.13 (m, 1H), 2.13-2.22 (m, 1H), 3.95 (d, J=6.41 Hz, 1H), 4.05 (dd, J=3.13, 6.48 Hz, 1H), 4.27 (s, 3H), 4.93-5.02 (m, 1H), 7.22 (dd, J=4.88, 7.63 Hz, 1H), 7.73 (d, J=2.90 Hz, 1H), 8.40 (dd, J=1.98, 4.88 Hz, 1H), 8.55 (dd, J=1.83, 7.48 Hz, 1H), 9.46 (s, 1H)

Example 436: N-(7-cyclobutoxy-8-fluoro-2-(1-methyl-2-oxabicyclo[2.1.1]hexan-4-yl)imidazo[1,2-a]pyridin-6-yl)-2-methoxynicotinamide 2,2,2-trifluoroacetate; LCMS (ESI) m/z 453.0 (M+H)⁺; ¹H NMR (500 MHz, METHANOL-d₄) δ ppm: 1.54 (s, 4H), 1.62 (s, 1H), 1.72-1.84 (m, 2H), 2.01 (dd, J=1.75, 4.65 Hz, 3H), 2.24 (dd, J=1.37, 4.73 Hz, 3H), 2.42-2.54 (m, 2H), 2.59-2.69 (m, 2H), 3.68-3.73 (m, 1H), 4.06 (s, 2H), 4.28-4.31 (m, 3H), 7.26 (dd, J=4.88, 7.48 Hz, 1H), 8.06-8.12 (m, 1H), 8.46 (dd, J=1.91, 4.81 Hz, 1H), 8.60 (dd, J=1.98, 7.78 Hz, 1H), 9.81 (s, 1H)

Example 437: N-(7-(cyclopentyloxy)-8-fluoro-2-(1-methyl-2-oxabicyclo[2.1.1]hexan-4-yl)imidazo[1,2-a]pyridin-6-yl)-1-methyl-1H-pyrazole-3-carboxamide 2,2,2-trifluoroacetate; LCMS (ESI) m/z 440.0 (M+H)⁺; ¹H NMR (500 MHz, METHANOL-d₄) δ ppm: 1.52 (s, 3H), 1.76-1.86 (m, 2H), 1.95-2.07 (m, 7H), 2.09-2.17 (m, 2H), 2.20 (dd, J=1.68, 4.58 Hz, 2H), 4.00 (s, 3H), 4.04 (s, 2H), 5.50-5.58 (m, 1H), 6.86 (d, J=2.44 Hz, 1H), 7.74 (d, J=2.44 Hz, 1H), 8.02 (d, J=2.14 Hz, 1H), 9.50 (s, 1H),

Example 438: 6-ethyl-N-(2-(tetrahydro-2H-pyran-4-yl)imidazo[1,2-a]pyrazin-6-yl)picolinamide; LCMS (ESI) m/z 404.2 (M+H)⁺; ¹H NMR (400 MHz, METHANOL-d₄) δ ppm: 1.40 (t, J=7.6 Hz, 3H), 1.83-1.95 (m, 2H), 1.99-2.07 (m, 2H), 2.96 (q, J=7.6 Hz, 2H), 3.06-3.15 (m, 1H), 3.62 (td, J=11.6 Hz, 2.4 Hz, 2H), 4.02-4.08 (m, 2H), 7.54 (d, J=7.6 Hz, 1H), 7.92-7.99 (m, 2H), 8.08 (d, J=7.6 Hz, 1H), 8.81 (d, J=0.8 Hz, 1H), 9.44 (d, J=1.6 Hz, 1H)

Example 439: 2-(difluoromethyl)-N-(8-methoxy-2-(1-methyl-2-oxabicyclo[2.1.1]hexan-4-yl)imidazo[1,2-a]pyrazin-6-yl)oxazole-4-carboxamide; LCMS (ESI) m/z 406.2 (M+H); ¹H NMR (500 MHz, DMSO-d₆) δ ppm 1.43 (s, 3H), 1.77 (dd, J=4.27, 1.22 Hz, 2H), 2.01 (d, J=4.27 Hz, 2H), 3.90 (s, 2H), 4.08 (s, 3H), 7.26-7.49 (m, 1H), 8.12 (s, 1H), 8.88 (s, 1H), 9.14 (s, 1H), 9.95 (s, 1H)

Example 440: 2-ethoxy-N-(8-methoxy-2-(1-methyl-2-oxabicyclo[2.1.1]hexan-4-yl)imidazo[1,2-a]pyrazin-6-yl)oxazole-4-carboxamide; LCMS (ESI) m/z 400.3 (M+H)⁺; ¹H NMR (500 MHz, DMSO-d₆) δ ppm 1.40-1.43 (m, 7H), 1.77 (dd, J=4.27, 1.22 Hz, 2H), 2.01 (dd, J=4.27, 1.83 Hz, 2H), 3.89 (s, 2H), 4.07 (s, 4H), 4.51-4.58 (m, 3H), 8.12 (s, 1H), 8.44 (s, 1H), 8.85 (s, 1H), 9.28 (s, 1H)

Example 441: N-(8-methoxy-2-(1-methyl-2-oxabicyclo[2.1.1]hexan-4-yl)imidazo[1,2-a]pyrazin-6-yl)-1-methyl-1H-pyrazole-3-carboxamide; LCMS (ESI) m/z 369.2 (M+H); ¹H NMR (500 MHz, DMSO-d₆) δ ppm 1.43 (s, 3H), 1.77 (dd, J=4.27, 1.22 Hz, 2H), 2.02 (dd, J=4.27, 1.83 Hz, 2H), 3.90 (s, 2H), 3.98 (s, 3H), 4.08 (s, 3H), 6.84 (d, J=2.44 Hz, 1H), 7.90 (d, J=2.44 Hz, 1H), 8.12 (s, 1H), 8.87 (s, 1H), 9.28 (s, 1H)

Example 442: N-(8-methoxy-2-(1-methyl-2-oxabicyclo[2.1.1]hexan-4-yl)imidazo[1,2-a]pyrazin-6-yl)pyrazolo[1,5-a]pyridine-7-carboxamide; LCMS (ESI) m/z 405.3 (M+H); ¹H NMR (500 MHz, DMSO-d₆) δ ppm 1.44 (s, 5H), 1.78 (dd, J=4.27, 1.83 Hz, 3H), 2.00-2.05 (m, 3H), 3.91 (s, 3H), 4.12 (s, 5H), 6.99 (d, J=2.44 Hz, 1H), 7.51 (dd, J=8.55, 7.32 Hz, 1H), 7.97 (dd, J=7.32, 1.22 Hz, 1H), 8.11 (d, J=1.22 Hz, 1H), 8.16 (s, 1H), 8.37 (d, J=2.44 Hz, 1H), 9.12 (s, 1H), 12.99 (s, 2H)

Example 443: 2-methoxy-N-(8-methoxy-2-(1-methyl-2-oxabicyclo[2.1.1]hexan-4-yl)imidazo[1,2-a]pyrazin-6-yl)benzamide; LCMS (ESI) m/z 395.3 (M+H)⁺; ¹H NMR (500 MHz, DMSO-d₆) δ ppm 1.44 (s, 3H), 1.78 (dd, J=3.97, 1.53 Hz, 2H), 2.02 (dd, J=4.27, 1.83 Hz, 2H), 3.90 (s, 2H), 4.02 (s, 3H), 4.07 (s, 2H), 7.07-7.18 (m, 1H), 7.27 (d, J=8.55 Hz, 1H), 7.53-7.63 (m, 1H), 7.91 (dd, J=7.32, 1.83 Hz, 1H), 8.11 (s, 1H), 8.99 (s, 1H), 10.27 (s, 1H).

Example 444: 2-fluoro-N-(8-methoxy-2-(1-methyl-2-oxabicyclo[2.1.1]hexan-4-yl)imidazo[1,2-a]pyrazin-6-yl)-3-methylbenzamide; LCMS (ESI) m/z 397.2 (M+H)⁺; ¹H NMR (500 MHz, DMSO-d₆) δ ppm 1.44 (s, 4H), 1.78 (dd, J=4.58, 1.53 Hz, 2H), 2.02 (dd, J=4.58, 1.53 Hz, 2H), 2.31 (s, 4H), 3.90 (s, 2H), 4.01-4.05 (m, 1H), 4.05 (s, 1H), 7.20-7.25 (m, 1H), 7.43-7.52 (m, 2H), 8.11 (s, 1H), 8.97 (s, 1H), 10.60 (s, 1H)

Example 445: N-(8-methoxy-2-(1-methyl-2-oxabicyclo[2.1.1]hexan-4-yl)imidazo[1,2-a]pyrazin-6-yl)-2,3-dihydrobenzofuran-7-carboxamide 2,2,2-trifluoroacetate; LCMS (ESI) m/z 407.2 (M+H); ¹H NMR (500 MHz, DMSO-d₆) δ ppm 1.43 (s, 3H), 1.77 (dd, J=3.97, 1.53 Hz, 2H), 2.02 (dd, J=4.27, 1.22 Hz, 2H), 3.32 (t, J=8.55 Hz, 1H), 3.90 (s, 2H), 4.07 (s, 3H), 4.87 (t, J=8.55 Hz, 2H), 6.98-7.19 (m, 2H), 7.52 (dd, J=7.32, 1.22 Hz, 1H), 7.77 (d, J=7.32 Hz, 1H), 8.11 (s, 1H), 9.01 (s, 1H), 9.73 (s, 1H).

Example 446: 3,3-difluoro-N-(8-methoxy-2-(1-methyl-2-oxabicyclo[2.1.1]hexan-4-yl)imidazo[1,2-a]pyrazin-6-yl)-2,3-dihydrobenzofuran-7-carboxamide; LCMS (ESI) m/z 443.1 (M+H)⁺; ¹H NMR: (500 MHz, METHANOL-d₄) δ ppm: 1.51 (s, 3H), 1.80-1.90 (m, 2H), 2.10-2.20 (m, 2H), 4.04 (s, 2H), 4.18 (s, 3H), 5.02 (t, J=16.0 Hz, 2H), 7.30-7.40 (m, 1H), 7.70-7.80 (m, 1H), 7.91 (m, 1H), 8.20-8.30 (m, 1H), 9.03 (s, 1H)

Example 447: 2-methoxy-N-(8-methoxy-2-(1-methyl-2-oxabicyclo[2.1.1]hexan-4-yl)imidazo[1,2-a]pyrazin-6-yl)nicotinamide 2,2,2-trifluoroacetate; LCMS (ESI) m/z 396.3 (M+H)⁺; ¹H NMR (500 MHz, DMSO-d₆) δ ppm 1.44 (s, 3H), 1.78 (dd, J=4.27, 1.22 Hz, 2H), 2.03 (dd, J=4.27, 1.83 Hz, 2H), 3.90 (s, 2H), 4.08 (d, J=4.27 Hz, 5H), 7.21 (dd, J=7.94, 4.88 Hz, 1H), 8.13 (s, 1H), 8.23 (dd, J=7.32, 1.83 Hz, 1H), 8.39 (dd, J=4.88, 1.83 Hz, 1H), 8.99 (s, 1H), 10.32 (s, 1H).

Example 448: 6-methoxy-N-(8-methoxy-2-(1-methyl-2-oxabicyclo[2.1.1]hexan-4-yl)imidazo[1,2-a]pyrazin-6-yl)picolinamide 2,2,2-trifluoroacetate; LCMS (ESI) m/z 396.3 (M+H)⁺; ¹H NMR (500 MHz, DMSO-d₆) δ ppm 1.44 (s, 3H), 1.78 (dd, J=4.27, 1.83 Hz, 2H), 2.03 (dd, J=4.27, 1.83 Hz, 2H), 3.91 (s, 2H), 4.05 (s, 3H), 4.10 (s, 3H), 7.15-7.20 (m, 1H), 7.80 (d, J=7.32 Hz, 1H), 7.96-8.03 (m, 1H), 8.14 (s, 1H), 8.97 (s, 1H), 9.93 (s, 1H).

Example 449: N-(8-methoxy-2-(1-methyl-2-oxabicyclo[2.1.1]hexan-4-yl)imidazo[1,2-a]pyrazin-6-yl)-6-(trifluoromethyl)picolinamide 2,2,2-trifluoroacetate; LCMS (ESI) m/z 434.2 (M+H)⁺; ¹H NMR (400 MHz, CHLOROFORM-d) δ ppm: 1.58 (s, 3H), 2.07-2.13 (m, 2H), 2.29-2.34 (m, 2H), 4.15 (s, 2H), 4.28 (s, 3H), 7.65 (s, 1H), 7.99 (dd, J=7.78, 1.00 Hz, 1H), 8.21 (t, J=7.66 Hz, 1H), 8.50 (d, J=7.78 Hz, 1H), 9.19 (s, 1H), 10.13 (s, 1H)

Example 450: 6-(difluoromethyl)-N-(8-methoxy-2-(tetrahydro-2H-pyran-4-yl)imidazo[1,2-a]pyrazin-6-yl)picolinamide; LCMS (ESI) m/z 404.2 (M+H)⁺; ¹H NMR (500 MHz, DMSO-d₆) δ ppm 1.71 (td, J=12.06, 3.97 Hz, 2H), 1.91 (br dd, J=12.82, 1.83 Hz, 2H), 2.96 (ddd, J=11.44, 7.48, 3.66 Hz, 1H), 3.43-3.51 (m, 2H), 3.93 (dt, J=9.46, 1.98 Hz, 2H), 4.09 (s, 2H), 7.07-7.30 (m, 3H), 8.02-8.05 (m, 1H), 8.31-8.36 (m, 2H), 9.00 (s, 1H).

Example 451: N-(8-methoxy-2-(tetrahydro-2H-pyran-4-yl)imidazo[1,2-a]pyrazin-6-yl)-6-(trifluoromethyl)picolinamide 2,2,2-trifluoroacetate; LCMS (ESI) m/z 422.3 (M+H)⁺; ¹H NMR (500 MHz, DMSO-d₆) δ ppm 1.67-1.76 (m, 3H), 1.89-1.96 (m, 2H), 2.96 (ddt, J=15.26, 7.78, 3.74, 3.74 Hz, 2H), 3.47 (td, J=11.60, 1.83 Hz, 2H), 3.93 (dt, J=9.46, 1.98 Hz, 2H), 4.09 (s, 3H), 8.05 (s, 1H), 8.24 (br d, J=7.32 Hz, 1H), 8.39-8.44 (m, 1H), 8.44-8.48 (m, 1H), 8.99 (s, 1H)

Example 452: 1-(2,2-difluoroethyl)-N-(8-methoxy-2-(tetrahydro-2H-pyran-4-yl)imidazo[1,2-a]pyrazin-6-yl)-1H-pyrazole-3-carboxamide; LCMS (ESI) m/z 407.2 (M+H)⁺; ¹H NMR (500 MHz, DMSO-d₆) δ ppm: 1.66-1.75 (m, 2H), 1.88-1.95 (m, 2H), 2.95 (tt, J=11.44, 3.81 Hz, 1H), 3.46 (td, J=11.75, 2.14 Hz, 2H), 3.90-3.96 (m, 2H), 4.04-4.10 (m, 3H), 4.80 (td, J=15.26, 3.66 Hz, 3H), 6.37-6.61 (m, 3H), 6.92 (d, J=2.44 Hz, 1H), 7.96-8.03 (m, 2H), 8.86 (s, 1H)

Example 453: N-(8-methoxy-2-(tetrahydro-2H-pyran-4-yl)imidazo[1,2-a]pyrazin-6-yl)-2,3-dihydrobenzofuran-7-carboxamide; LCMS (ESI) m/z 395.2 (M+H)⁺; ¹H NMR (500 MHz, DMSO-d₆) δ ppm 1.67-1.73 (m, 2H), 1.90 (br dd, J=12.82, 2.44 Hz, 2H), 2.94 (ddd, J=15.26, 7.32, 3.66 Hz, 2H), 3.43-3.49 (m, 2H), 3.90-3.96 (m, 3H), 4.05-4.08 (m, 3H), 4.86 (br t, J=8.85 Hz, 2H), 7.03-7.08 (m, 1H), 7.50-7.53 (m, 1H), 7.76 (br d, J=6.71 Hz, 1H), 7.99-8.03 (m, 1H), 8.99 (s, 1H), 9.70 (s, 1H).

Example 454: 3,3-difluoro-N-(8-methoxy-2-(tetrahydro-2H-pyran-4-yl)imidazo[1,2-a]pyrazin-6-yl)-2,3-dihydrobenzofuran-7-carboxamide; LCMS (ESI) m/z 431.0 (M+H)⁺; ¹H NMR: (400 MHz, DMSO-d₆) δ ppm: 1.66-1.70 (m, 2H), 1.88-1.91 (m, 2H), 2.91-2.95 (m, 1H), 3.43-3.48 (m, 2H), 3.90-3.93 (m, 2H), 4.05 (s, 3H), 5.02-5.10 (t, J=16 Hz, 2H), 7.29-7.33 (m, 1H), 7.90 (d, J=7.6 Hz, 1H), 8.00 (s, 1H), 8.06 (d, J=7.6 Hz, 1H), 8.97 (s, 1H), 9.80 (s, 1H).

Example 455: N-(8-methoxy-2-(tetrahydro-2H-pyran-4-yl)imidazo[1,2-a]pyrazin-6-yl)-6-methylpicolinamide; ¹H NMR (500 MHz, DMSO-d₆) δ ppm: 1.62-1.72 (m, 2H), 1.86-1.96 (m, 2H), 2.64 (s, 3H), 2.98 (tt, J=11.52, 3.74 Hz, 1H), 3.48 (td, J=11.60, 2.44 Hz, 1H), 3.94 (dt, J=9.61, 1.91 Hz, 2H), 4.11 (s, 3H), 7.60 (dd, J=6.71, 1.83 Hz, 1H), 7.95-8.04 (m, 2H), 8.07 (s, 1H), 9.01 (s, 1H), 10.05 (s, 1H)

Example 456: 6-isopropyl-N-(8-methoxy-2-(tetrahydro-2H-pyran-4-yl)imidazo[1,2-a]pyrazin-6-yl)picolinamide; LCMS (ESI) m/z 396.3 (M+H)⁺; ¹H NMR (500 MHz, DMSO-d₆) δ ppm 1.34 (d, J=6.71 Hz, 6H), 1.70 (qd, J=12.21, 4.27 Hz, 2H), 1.92 (br d, J=13.43 Hz, 2H), 2.93-3.02 (m, 1H), 3.20 (dt, J=13.89, 6.79 Hz, 1H), 3.47 (t, J=11.29 Hz, 1H), 3.93 (br d, J=11.60 Hz, 2H), 4.10 (s, 3H), 7.62-7.70 (m, 1H), 7.98-8.09 (m, 2H), 8.99 (s, 1H), 10.09 (s, 1H)

Example 457: 2-methoxy-N-(8-methoxy-2-(tetrahydro-2H-pyran-4-yl)imidazo[1,2-a]pyrazin-6-yl)benzamide 2,2,2-trifluoroacetate; LCMS (ESI) m/z 383.2 (M+H)⁺; ¹H NMR (500 MHz, DMSO-d₆) δ ppm 1.66-1.81 (m, 2H), 1.92 (br dd, J=12.82, 2.44 Hz, 2H), 2.98 (tt, J=11.52, 3.74 Hz, 1H), 3.47 (td, J=11.60, 1.83 Hz, 1H), 3.94 (dt, J=9.46, 1.98 Hz, 2H), 4.02 (s, 3H), 4.09 (s, 3H), 7.13-7.21 (m, 1H), 7.27 (d, J=8.55 Hz, 1H), 7.56-7.65 (m, 1H), 7.91 (dd, J=7.63, 1.53 Hz, 1H), 8.07 (s, 1H), 9.02 (s, 1H), 10.31 (s, 1H).

Example 458: 2-methoxy-N-(8-methoxy-2-(tetrahydro-2H-pyran-4-yl)imidazo[1,2-a]pyrazin-6-yl)nicotinamide 2,2,2-trifluoroacetate; LCMS (ESI) m/z 384.2 (M+H)⁺; ¹H NMR (500 MHz, DMSO-d₆) δ ppm 1.68-1.74 (m, 2H), 1.92 (br dd, J=13.12, 2.14 Hz, 3H), 2.95-3.02 (m, 1H), 3.36-3.60 (m, 1H), 3.37-3.55 (m, 1H), 3.38-3.74 (m, 1H), 3.47 (td, J=11.60, 1.83 Hz, 1H), 3.89-3.97 (m, 3H), 4.07-4.12 (m, 6H), 7.21 (dd, J=7.32, 4.88 Hz, 1H), 8.07 (s, 1H), 8.23 (dd, J=7.63, 2.14 Hz, 1H), 8.40 (dd, J=4.88, 1.83 Hz, 1H), 9.01 (s, 1H), 10.35 (s, 1H)

Example 459: 2-ethoxy-N-(8-methoxy-2-(tetrahydro-2H-pyran-4-yl)imidazo[1,2-a]pyrazin-6-yl)nicotinamide; LCMS (ESI) m/z 398.3 (M+H)⁺; ¹H NMR (500 MHz, DMSO-d₆) δ ppm 1.50-1.55 (m, 3H), 1.65-1.74 (m, 2H), 1.88-1.93 (m, 2H), 2.90-2.97 (m, 1H), 3.43-3.52 (m, 1H), 3.92-3.96 (m, 2H), 4.06 (s, 2H), 4.56 (q, J=7.32 Hz, 2H), 7.24 (dd, J=7.94, 4.88 Hz, 1H), 8.02 (s, 1H), 8.34-8.42 (m, 2H), 8.97 (s, 1H), 10.42 (s, 1H).

Example 460: N-(8-methoxy-2-(tetrahydro-2H-pyran-4-yl)imidazo[1,2-a]pyrazin-6-yl)-2-propoxynicotinamide; LCMS (ESI) m/z 412.4 (M+H)⁺; ¹H NMR (500 MHz, DMSO-d₆) δ ppm 1.17 (t, J=7.32 Hz, 3H), 1.61-1.74 (m, 2H), 1.89-1.95 (m, 4H), 2.96 (tt, J=11.44, 3.81 Hz, 1H), 3.47 (td, J=11.60, 1.83 Hz, 1H), 3.93 (dt, J=9.46, 1.98 Hz, 2H), 4.05 (s, 2H), 4.49 (t, J=6.10 Hz, 2H), 7.25 (dd, J=7.32, 4.88 Hz, 1H), 8.03 (s, 1H), 8.38-8.42 (m, 1H), 9.00 (s, 1H), 10.34 (s, 1H)

Example 461: N-(8-methoxy-2-(tetrahydro-2H-pyran-4-yl)imidazo[1,2-a]pyrazin-6-yl)-1-methyl-1H-pyrazole-3-carboxamide Hydrochloride; LCMS (ESI) m/z 357.1 (M+H)⁺; ¹H NMR (500 MHz, METHANOL-d₄) δ: 1.88-1.91 (m, 2H), 2.03 (d, J=10.0 Hz, 2H), 3.20-3.24 (m, 1H), 3.60 (t, J=20.0 Hz, 2H), 4.02 (s, 3H), 4.05-4.08 (m, 2H), 4.31 (s, 3H), 6.88 (d, J=5.0 Hz, 1H), 7.73 (s, 1H), 8.23 (s, 1H), 9.20 (d, J=5.0 Hz, 1H)

Example 462: 1-isopropyl-N-(8-methoxy-2-(tetrahydro-2H-pyran-4-yl)imidazo[1,2-a]pyrazin-6-yl)-1H-pyrazole-3-carboxamide 2,2,2-trifluoroacetate; ¹H NMR (500 MHz, DMSO-d₆) δ ppm 1.50 (d, J=6.71 Hz, 7H), 1.65-1.73 (m, 2H), 1.92 (br dd, J=13.12, 2.14 Hz, 2H, 2.96-3.02 (m, 1H), 3.47 (td, J=11.60, 2.44 Hz, 1H), 3.58 (br s, 1H), 3.93 (dt, J=9.16, 2.14 Hz, 3H), 4.11 (s, 3H), 4.65 (spt, J=6.71 Hz, 1H), 6.85 (d, J=2.44 Hz, 1H), 7.99 (d, J=2.44 Hz, 1H), 8.09 (s, 1H), 8.91 (s, 1H), 9.33 (br s, 1H)

Example 463: 1-ethyl-N-(8-methoxy-2-(tetrahydro-2H-pyran-4-yl)imidazo[1,2-a]pyrazin-6-yl)-1H-pyrazole-3-carboxamide 2,2,2-trifluoroacetate; ¹H NMR (500 MHz, DMSO-d₆) δ ppm 1.45 (t, J=7.32 Hz, 3H), 1.65-1.73 (m, 2H), 1.92 (br dd, J=12.82, 2.44 Hz, 2H), 2.99 (tt, J=11.37, 3.89 Hz, 1H), 3.44-3.49 (m, 1H), 3.93 (dt, J=9.61, 1.91 Hz, 2H), 4.10 (s, 3H), 4.28 (q, J=7.32 Hz, 2H), 6.85 (d, J=2.44 Hz, 1H), 7.96 (d, J=2.44 Hz, 1H), 8.09 (s, 1H), 8.91 (s, 1H), 9.35 (br s, 1H)

Example 464: 1-isobutyl-N-(8-methoxy-2-(tetrahydro-2H-pyran-4-yl)imidazo[1,2-a]pyrazin-6-yl)-1H-pyrazole-3-carboxamide; ¹H NMR (500 MHz, DMSO-d₆) δ ppm 0.87 (d, J=6.71 Hz, 7H), 1.66-1.73 (m, 2H), 1.91 (br dd, J=12.82, 1.83 Hz, 2H), 2.16-2.24 (m, 1H), 2.90-3.02 (m, 1H), 3.47 (td, J=11.60, 2.44 Hz, 1H), 3.90-3.98 (m, 2H), 4.04-4.09 (m, 5H), 6.85 (d, J=2.44 Hz, 1H), 7.93 (d, J=2.44 Hz, 1H), 8.04 (s, 1H), 8.87 (s, 1H), 9.24 (s, 1H)

Example 465: 2-methoxy-N-(8-methoxy-2-(1-methyl-2-oxabicyclo[2.2.1]heptan-4-yl)imidazo[1,2-a]pyrazin-6-yl)benzamide 2,2,2-trifluoroacetate; LCMS (ESI) m/z 409.2 (M+H)⁺; ¹H NMR (500 MHz, DMSO-d₆) δ ppm 1.39 (s, 4H), 1.64-1.71 (m, 1H), 1.76-1.88 (m, 4H), 1.94-1.98 (m, 1H), 2.04-2.10 (m, 1H), 3.79 (d, J=6.10 Hz, 1H), 3.93 (dd, J=6.41, 3.36 Hz, 1H), 4.03 (s, 3H), 4.08 (s, 3H), 7.14 (t, J=7.94 Hz, 1H), 7.27 (d, J=8.55 Hz, 1H), 7.59 (td, J=7.94, 1.83 Hz, 1H), 7.91 (dd, J=7.63, 1.53 Hz, 1H), 8.11 (s, 1H), 8.99 (s, 1H), 10.28 (s, 1H).

Example 466: 3-fluoro-2-methoxy-N-(8-methoxy-2-(1-methyl-2-oxabicyclo[2.2.1]heptan-4-yl)imidazo[1,2-a]pyrazin-6-yl)benzamide 2,2,2-trifluoroacetate; LCMS (ESI) m/z 427.3 (M+H)⁺; ¹H NMR (500 MHz, DMSO-d₆) δ ppm 1.39 (s, 4H), 1.66-1.74 (m, 1H), 1.76-1.87 (m, 4H), 1.94-1.98 (m, 1H), 2.01-2.11 (m, 1H), 3.79 (d, J=6.10 Hz, 1H), 3.93 (dd, J=6.41, 3.36 Hz, 1H), 3.99 (d, J=1.22 Hz, 3H), 4.06 (s, 3H), 7.24 (td, J=7.94, 4.88 Hz, 1H), 7.42-7.53 (m, 2H), 8.11 (s, 1H), 8.97 (s, 1H), 10.53 (s, 1H)

Example 467: 2-fluoro-N-(8-methoxy-2-(1-methyl-2-oxabicyclo[2.2.1]heptan-4-yl)imidazo[1,2-a]pyrazin-6-yl)-3-methylbenzamide 2,2,2-trifluoroacetate; LCMS (ESI) m/z 411.3 (M+H); ¹H NMR (500 MHz, DMSO-d₆) δ ppm 1.39 (s, 5H), 1.66-1.73 (m, 2H), 1.74-1.88 (m, 5H), 1.94-1.98 (m, 2H), 2.02-2.11 (m, 2H), 2.31 (d, J=1.22 Hz, 4H), 3.78 (d, J=6.71 Hz, 1H), 3.89-3.97 (m, 1H), 4.05 (s, 3H), 7.18-7.27 (m, 1H), 7.40-7.51 (m, 2H), 8.10 (s, 1H), 8.96 (s, 1H), 10.60 (s, 1H)

Example 468: 2-methoxy-N-(8-methoxy-2-(1-methyl-2-oxabicyclo[2.2.1]heptan-4-yl)imidazo[1,2-a]pyrazin-6-yl)nicotinamide 2,2,2-trifluoroacetate; LCMS (ESI) m/z 410.2 (M+H)⁺; ¹H NMR (500 MHz, DMSO-d₆) δ ppm 1.39 (s, 3H), 1.66-1.81 (m, 2H), 1.82-1.88 (m, 2H), 1.91-1.98 (m, 1H), 2.02-2.10 (m, 1H), 3.78 (d, J=6.10 Hz, 1H), 3.93 (dd, J=6.41, 3.36 Hz, 1H), 4.08 (d, J=2.44 Hz, 5H), 7.18-7.26 (m, 1H), 8.10 (s, 1H), 8.23 (dd, J=7.32, 1.83 Hz, 1H), 8.39 (dd, J=4.88, 1.83 Hz, 1H), 8.98 (s, 1H), 10.31 (s, 1H)

Example 469: N-(8-methoxy-2-(1-methyl-2-oxabicyclo[2.2.1]heptan-4-yl)imidazo[1,2-a]pyrazin-6-yl)-2,3-dihydrobenzofuran-7-carboxamide 2,2,2-trifluoroacetate; LCMS (ESI) m/z 421.2 (M+H)⁺; ¹H NMR (500 MHz, DMSO-d₆) δ ppm 1.39 (s, 3H), 1.64-1.74 (m, 1H), 1.75-1.90 (m, 3H), 1.93-1.97 (m, 1H), 2.01-2.11 (m, 1H), 3.32 (t, J=8.55 Hz, 1H), 3.78 (d, J=6.10 Hz, 1H), 3.92 (dd, J=6.41, 3.36 Hz, 1H), 4.07 (s, 3H), 4.87 (t, J=8.85 Hz, 2H), 6.99-7.09 (m, 1H), 7.52 (dd, J=7.32, 1.22 Hz, 1H), 7.77 (d, J=6.71 Hz, 1H), 8.10 (s, 1H), 9.00 (s, 1H), 9.73 (s, 1H)

Example 470: N-(8-methoxy-2-(1-methyl-2-oxabicyclo[2.2.1]heptan-4-yl)imidazo[1,2-a]pyrazin-6-yl)pyrazolo[1,5-a]pyridine-7-carboxamide 2,2,2-trifluoroacetate; LCMS (ESI) m/z 419.3 (M+H); ¹H NMR (500 MHz, DMSO-d₆) δ ppm 1.39 (s, 3H), 1.66-1.74 (m, 1H), 1.78-1.85 (m, 2H), 1.85-1.89 (m, 1H), 1.95-1.99 (m, 1H), 2.04-2.13 (m, 1H), 3.79 (d, J=6.10 Hz, 1H), 3.91-3.96 (m, 1H), 4.12 (s, 3H), 6.98-7.01 (m, 1H), 7.51 (dd, J=8.85, 7.02 Hz, 1H), 7.97 (dd, J=7.02, 1.53 Hz, 1H), 8.10 (dd, J=8.55, 1.22 Hz, 1H), 8.15 (s, 1H), 8.37 (d, J=2.44 Hz, 1H), 9.12 (s, 1H), 13.00 (s, 1H)

Example 471: N-(8-methoxy-2-(1-methyl-2-oxabicyclo[2.2.1]heptan-4-yl)imidazo[1,2-a]pyrazin-6-yl)-2-methyloxazole-4-carboxamide 2,2,2-trifluoroacetate; LCMS (ESI) m/z 384.2 (M+H)⁺; ¹H NMR (500 MHz, DMSO-d₆) δ ppm 1.38 (s, 3H), 1.61-1.74 (m, 1H), 1.75-1.89 (m, 3H), 1.90-1.96 (m, 1H), 2.02-2.11 (m, 1H), 2.53 (s, 4H), 3.77 (d, J=6.71 Hz, 1H), 3.89-3.96 (m, 1H), 4.07 (s, 3H), 8.10 (s, 1H), 8.73-8.88 (m, 1H), 9.41 (s, 1H)

Example 472: 2-cyclopropyl-N-(8-methoxy-2-(1-methyl-2-oxabicyclo[2.2.1]heptan-4-yl)imidazo[1,2-a]pyrazin-6-yl)oxazole-4-carboxamide 2,2,2-trifluoroacetate; LCMS (ESI) m/z 410.2 (M+H)⁺; ¹H NMR (500 MHz, DMSO-d₆) δ ppm 1.03-1.16 (m, 4H), 1.38 (s, 3H), 1.66-1.73 (m, 1H), 1.75-1.87 (m, 3H), 1.90-1.99 (m, 1H), 2.02-2.11 (m, 1H), 2.19-2.26 (m, 1H), 3.78 (d, J=6.71 Hz, 1H), 3.86-3.95 (m, 1H), 4.08 (s, 3H), 8.10 (s, 1H), 8.69-8.87 (m, 1H), 9.37 (s, 1H)

Example 473: N-(8-ethoxy-2-(1-methyl-2-oxabicyclo[2.1.1]hexan-4-yl)imidazo[1,2-a]pyrazin-6-yl)-2-methyloxazole-4-carboxamide; LCMS (ESI) m/z 384.2 (M+H)⁺; ¹H NMR (500 MHz, METHANOL-d₄) δ ppm: 1.52 (s, 3H), 1.56 (t, J=7.5 Hz, 3H), 1.80-1.90 (m, 2H), 2.10-2.20 (m, 2H), 2.56 (s, 3H), 4.05 (s, 2H), 4.60-4.70 (m, 2H), 7.89 (s, 1H), 8.46 (s, 1H), 8.92 (s, 1H)

Example 474: N-(8-ethoxy-2-(tetrahydro-2H-pyran-4-yl)imidazo[1,2-a]pyrazin-6-yl)-6-methylpicolinamide 2,2,2-trifluoroacetate; ¹H NMR (500 MHz, DMSO-d₆) δ ppm 1.46 (t, J=7.02 Hz, 3H), 1.65-1.76 (m, 2H), 1.89-1.95 (m, 2H), 2.64 (s, 3H), 2.98 (tt, J=11.44, 4.12 Hz, 1H), 3.47 (td, J=11.60, 1.83 Hz, 1H), 3.94 (dt, J=9.46, 2.29 Hz, 2H), 4.57 (q, J=7.32 Hz, 2H), 7.58-7.63 (m, 1H), 7.96-8.03 (m, 1H), 8.02-8.09 (m, 1H), 9.01 (s, 1H), 10.03 (s, 1H).

Example 475: 6-(difluoromethyl)-N-(8-ethoxy-2-(tetrahydro-2H-pyran-4-yl)imidazo[1,2-a]pyrazin-6-yl)picolinamide 2,2,2-trifluoroacetate; LCMS (ESI) m/z 418.3 (M+H)⁺; ¹H NMR (500 MHz, DMSO-d₆) δ ppm 1.46 (t, J=7.02 Hz, 3H), 1.66-1.76 (m, 1H), 1.72-1.74 (m, 1H) 1.86-1.96 (m, 2H), 2.96-3.03 (m, 1H), 3.48 (td, J=11.60, 1.83 Hz, 1H), 3.94 (dt, J=9.61, 1.91 Hz, 2H), 4.57 (q, J=6.92 Hz, 2H), 7.06-7.32 (m, 1H), 8.03 (dd, J=7.02, 1.53 Hz, 1H), 8.08 (s, 1H), 8.32-8.37 (m, 2H), 9.02 (s, 1H), 9.97 (s, 1H)

Example 476: 6-(difluoromethyl)-N-(8-propoxy-2-(tetrahydro-2H-pyran-4-yl)imidazo[1,2-a]pyrazin-6-yl)picolinamide 2,2,2-trifluoroacetate; LCMS (ESI) m/z 432.3 (M+H)⁺; ¹H NMR (500 MHz, DMSO-d₆) δ ppm 1.04 (t, J=7.63 Hz, 3H), 1.67-1.74 (m, 2H), 1.85-1.95 (m, 3H), 2.94-3.01 (m, 1H), 3.46 (br d, J=9.77 Hz, 1H), 3.92-3.98 (m, 2H), 4.47 (t, J=6.71 Hz, 2H), 7.07-7.31 (m, 1H), 8.04-8.08 (m, 1H), 8.31-8.37 (m, 1H), 9.00 (s, 1H), 9.95 (s, 1H).

Example 477: N-(8-isopropoxy-2-(1-methyl-2-oxabicyclo[2.1.1]hexan-4-yl)imidazo[1,2-a]pyrazin-6-yl)-2-methoxybenzamide 2,2,2-trifluoroacetate; LCMS (ESI) m/z 423.4 (M+H)⁺; ¹H NMR (500 MHz, DMSO-d₆) δ ppm 1.39-1.49 (m, 1OH), 1.78 (dd, J=4.58, 1.53 Hz, 2H), 2.02 (dd, J=4.58, 1.53 Hz, 2H), 3.90-4.04 (m, 4H), 5.43-5.54 (m, 1H), 7.14 (t, J=7.94 Hz, 1H), 7.26 (d, J=8.55 Hz, 1H), 7.49-7.65 (m, 1H), 7.88 (dd, J=7.63, 1.53 Hz, 1H), 8.08 (s, 1H), 8.94 (s, 1H), 10.24 (s, 1H)

Example 478: 2-fluoro-N-(8-isopropoxy-2-(1-methyl-2-oxabicyclo[2.1.1]hexan-4-yl)imidazo[1,2-a]pyrazin-6-yl)-3-methylbenzamide 2,2,2-trifluoroacetate; LCMS (ESI) m/z 425.4 (M+H); ¹H NMR (500 MHz, DMSO-d₆) δ ppm 1.35-1.46 (m, 13H), 1.76-1.80 (m, 3H), 2.00-2.04 (m, 3H), 2.31 (s, 4H), 3.84-3.92 (m, 2H), 5.44-5.54 (m, 1H), 7.18-7.24 (m, 1H), 7.43-7.53 (m, 2H), 8.04-8.15 (m, 1H), 8.90-9.10 (m, 1H), 10.56 (s, 1H)

Example 479: 4-fluoro-N-(8-isopropoxy-2-(1-methyl-2-oxabicyclo[2.1.1]hexan-4-yl)imidazo[1,2-a]pyrazin-6-yl)-2-methoxybenzamide 2,2,2-trifluoroacetate; LCMS (ESI) m/z 441.2 (M+H); ¹H NMR (500 MHz, DMSO-d₆) δ ppm 1.41-1.45 (m, 7H) 1.72-1.84 (m, 1H) 2.00-2.06 (m, 1H) 3.90 (s, 4H) 3.97-4.05 (m, 6H) 5.48 (dt, J=12.36, 6.33 Hz, 1H) 6.97 (td, J=8.24, 2.44 Hz, 1H) 7.15-7.23 (m, 1H) 7.91 (t, J=7.94 Hz, 1H) 8.07 (s, 1H) 8.91 (s, 1H) 10.13 (s, 1H)

Example 480: N-(8-isopropoxy-2-(1-methyl-2-oxabicyclo[2.1.1]hexan-4-yl)imidazo[1,2-a]pyrazin-6-yl)-[1,2,4]triazolo[1,5-a]pyridine-5-carboxamide; LCMS (ESI) m/z 434.2 (M+H)⁺; ¹H NMR (500 MHz, METHANOL-d₄) δ ppm: 1.51 (s, 3H), 1.53 (d, J=6.5 Hz, 6H), 1.90-1.91 (m, 2H), 2.16 (d, J=6.0 Hz, 2H), 4.05 (s, 2H), 5.65-5.67 (m, 2H), 7.90-7.93 (m, 2H), 8.10 (d, J=7.5, 1H), 8.23 (d, J=7.5, 1H), 8.74 (s, 1H), 9.09 (s, 1H).

Example 481: N-(8-isopropoxy-2-(1-methyl-2-oxabicyclo[2.1.1]hexan-4-yl)imidazo[1,2-a]pyrazin-6-yl)pyrazolo[1,5-a]pyrimidine-7-carboxamide; LCMS (ESI) m/z 434.1 (M+H)⁺; ¹H NMR (400 MHz, CHLOROFORM-d) δ ppm: 1.53 (s, 3H), 1.54 (d, J=6.0 Hz, 6H), 1.98-2.00 (m, 2H), 2.10-2.12 (m, 2H), 4.10 (s, 2H), 5.65-5.72 (m, 1H), 6.97 (d, J=2.0 Hz, 1H), 7.50 (s, 1H), 7.90 (d, J=3.6 Hz, 1H), 8.36 (s, 1H), 8.75 (d, J=4.4 Hz, 1H), 8.97 (s, 1H), 12.81 (s, 1H).

Example 482: 4-fluoro-N-(8-isopropoxy-2-(1-methyl-2-oxabicyclo[2.1.1]hexan-4-yl)imidazo[1,2-a]pyrazin-6-yl)pyrazolo[1,5-a]pyridine-3-carboxamide; LCMS (ESI) m/z 451.2 (M+H)⁺; ¹H NMR (500 MHz, METHANOL-d₄) δ ppm: 1.49 (s, 3H), 1.50 (d, J=1 Hz, 6H), 1.88-1.90 (m, 2H), 2.14-2.16 (m, 2H), 4.04 (s, 2H), 5.59-5.65 (m, 1H), 7.05-7.09 (m, 1H), 7.28-7.32 (m, 1H), 7.86 (s, 1H), 8.57 (d, J=7 Hz, 1H), 8.50 (s, 1H), 8.91 (s, 1H).

Example 483: N-(2-(1-(fluoromethyl)-2-oxabicyclo[2.1.1]hexan-4-yl)-8-isopropoxyimidazo[1,2-a]pyrazin-6-yl)-2-methoxybenzamide 2,2,2-trifluoroacetate; LCMS (ESI) m/z 441.3 (M+H); ¹H NMR (500 MHz, DMSO-d₆) δ ppm 1.43 (d, J=6.10 Hz, 6H), 1.88 (dd, J=4.27, 1.83 Hz, 2H), 2.18 (dd, J=4.58, 1.53 Hz, 2H), 4.00 (d, J=7.94 Hz, 6H), 4.64-4.76 (m, 2H), 5.43-5.53 (m, 1H), 6.93-7.19 (m, 1H), 7.26 (d, J=8.55 Hz, 1H), 7.58 (td, J=7.94, 1.83 Hz, 1H), 7.85-7.89 (m, 1H), 8.14 (s, 1H), 8.95 (s, 1H), 10.26 (s, 1H).

Example 484: N-(8-cyclobutoxy-2-(1-methyl-2-oxabicyclo[2.1.1]hexan-4-yl)imidazo[1,2-a]pyrazin-6-yl)-2-methyloxazole-4-carboxamide; LCMS (ESI) m/z 410.2 (M+H)⁺; ¹H NMR (500 MHz, DMSO-d₆) δ ppm 1.43 (s, 3H), 1.63-1.74 (m, 1H), 1.75-1.80 (m, 2H), 1.79-1.90 (m, 1H), 2.02 (dd, J=4.58, 1.53 Hz, 2H), 2.16-2.27 (m, 2H), 2.53 (s, 4H), 3.90 (s, 2H), 5.32-5.39 (m, 1H), 8.10 (s, 1H), 8.73-8.86 (m, 1H), 9.40 (s, 1H)

Example 485: N-(8-cyclobutoxy-2-(1-methyl-2-oxabicyclo[2.1.1]hexan-4-yl)imidazo[1,2-a]pyrazin-6-yl)-2-(difluoromethyl)oxazole-4-carboxamide 2,2,2-trifluoroacetate; LCMS (ESI) m/z 446.2 (M+H)⁺; 1H NMR (500 MHz, DMSO-d₆) δ ppm 1.43 (s, 3H), 1.64-1.73 (m, 1H), 1.78 (dd, J=4.58, 1.53 Hz, 2H), 1.80-1.88 (m, 1H), 2.02 (dd, J=4.27, 1.83 Hz, 2H), 2.14-2.26 (m, 2H), 3.90 (s, 2H), 5.39 (quin, J=7.48 Hz, 1H), 7.25-7.49 (m, 1H), 8.11 (s, 1H), 8.85-9.15 (m, 1H), 9.90 (s, 1H),

Example 486: N-(8-cyclobutoxy-2-(1-methyl-2-oxabicyclo[2.2.1]heptan-4-yl)imidazo[1,2-a]pyrazin-6-yl)-2-methyloxazole-4-carboxamide 2,2,2-trifluoroacetate; LCMS (ESI) m/z 424.3 (M+H)⁺; ¹H NMR (500 MHz, DMSO-d₆) δ ppm 1.38 (s, 3H), 1.66-1.89 (m, 7H), 1.93-2.00 (m, 1H), 2.03-2.10 (m, 1H), 2.16-2.25 (m, 2H), 3.78 (d, J=6.71 Hz, 1H), 3.91 (dd, J=6.41, 3.36 Hz, 1H), 5.32-5.39 (m, 1H), 8.08 (s, 1H), 8.70-8.88 (m, 1H), 9.38 (s, 1H)

Example 487: 3,3-difluoro-N-(7-isopropoxy-2-(tetrahydro-2H-pyran-4-yl)imidazo[1,2-a]pyrimidin-6-yl)-2,3-dihydrobenzofuran-7-carboxamide; LCMS (ESI) m/z 459.0 (M+H)⁺; 1H NMR (400 MHz, METHANOL-d₄) δ: 1.46 (d, J=6.0 Hz, 6H), 1.64-1.67 (m, 2H), 1.86-1.89 (m, 2H), 2.80-2.90 (m, 1H), 3.45-3.48 (m, 2H), 3.89-3.93 (m, 2H), 5.14-5.22 (m, 2H), 5.33-5.37 (m, 1H), 7.36-7.39 (m, 1H), 7.56 (s, 1H), 7.98 (d, J=8.0 Hz, 1H), 8.20 (d, J=7.6 Hz, 1H), 9.65 (s, 1H), 9.79 (s, 1H).

Example 488: N-(7-isopropoxy-2-(1-methyl-2-oxabicyclo[2.1.1]hexan-4-yl)imidazo[1,2-a]pyrimidin-6-yl)-2-methoxybenzamide; LCMS (ESI) m/z 423.0 (M+H)⁺; ¹H NMR (400 MHz, METHANOL-d₄) δ: 1.49 (s, 3H), 1.54 (d, J=6.0 Hz, 6H), 1.84 (d, J=4.4 Hz, 2H), 2.09 (d, J=3.2 Hz, 2H), 3.99 (s, 2H), 4.13 (s, 3H), 5.55-5.62 (m, 1H), 7.10-7.15 (m, 1H), 7.23 (d, J=8.4 Hz, 1H), 7.45 (s, 1H), 7.55-7.59 (m, 1H), 8.15 (d, J=8.0 Hz, 1H), 9.68 (s, 1H)

Example 489: 3-fluoro-N-(7-isopropoxy-2-(1-methyl-2-oxabicyclo[2.1.1]hexan-4-yl)imidazo[1,2-a]pyrimidin-6-yl)-2-methoxybenzamide 2,2,2-trifluoroacetate; ¹H NMR (500 MHz, DMSO-d₆) δ ppm 1.45 (s, 4H), 1.51 (d, J=6.10 Hz, 7H), 1.83 (br d, J=2.44 Hz, 2H), 2.10 (br s, 2H), 3.92 (s, 2H), 4.15 (d, J=1.83 Hz, 3H), 5.37-5.65 (m, 1H), 7.35 (d, J=4.88 Hz, 1H), 7.52-7.76 (m, 1H), 7.85 (dd, J=7.94, 1.22 Hz, 1H), 7.90-8.11 (m, 1H), 9.70-10.06 (m, 1H), 10.51 (br s, 1H)

Example 490: 2-fluoro-N-(7-isopropoxy-2-(1-methyl-2-oxabicyclo[2.1.1]hexan-4-yl)imidazo[1,2-a]pyrimidin-6-yl)-3-methylbenzamide; LCMS (ESI) m/z 425.1 (M+H)⁺; ¹H NMR (400 MHz, METHANOL-d₄) δ ppm: 1.40-1.50 (m, 9H), 1.80-1.90 (m, 2H), 2.00-2.10 (m, 2H), 2.39 (s, 3H), 4.00 (s, 2H), 5.50-5.60 (m, 1H), 7.26 (t, J=8.0 Hz, 1H), 7.50-7.60 (m, 2H), 7.85 (t, J=7.2 Hz, 1H), 9.55 (s, 1H)

Example 491: N-(7-isopropoxy-2-(1-methyl-2-oxabicyclo[2.1.1]hexan-4-yl)imidazo[1,2-a]pyrimidin-6-yl)-6-methylpicolinamide; LCMS (ESI) m/z 408.1 (M+H)⁺; ¹H NMR (500 MHz, METHANOL-d₄) δ 1.49 (s, 3H), 1.54 (d, J=6.5 Hz, 6H), 1.80-1.90 (m, 2H), 2.00-2.10 (m, 2H), 2.64 (s, 3H), 3.99 (s, 2H), 5.50-5.60 (m, 1H), 7.40-7.50 (m, 2H), 7.90 (t, J=7.5 Hz, 1H), 8.01 (t, J=7.5 Hz, 1H), 9.61 (s, 1H)

Example 492: 6-cyclopropyl-N-(7-isopropoxy-2-(1-methyl-2-oxabicyclo[2.1.1]hexan-4-yl)imidazo[1,2-a]pyrimidin-6-yl)picolinamide; LCMS (ESI) m/z 434.1 (M+H)⁺; ¹H NMR (500 MHz, METHANOL-d₄) δ ppm: 1.49 (s, 3H), 1.56-1.57 (m, 6H), 1.84-1.87 (m, 2H), 2.07-2.11 (m, 2H), 3.99 (s, 2H), 2.20-2.24 (m, 1H), 5.56-5.63 (m, 1H), 7.49-7.56 (m, 2H), 7.85-7.87 (t, J=10.0 Hz, 1H), 7.93-7.95 (t, J=10.0 Hz, 1H), 9.63-9.65 (d, J=10.0 Hz, 1H)

Example 493: N-(7-isopropoxy-2-(1-methyl-2-oxabicyclo[2.1.1]hexan-4-yl)imidazo[1,2-a]pyrimidin-6-yl)-6-(tetrahydrofuran-3-yl)picolinamide; LCMS (ESI) m/z 464.1 (M+H)⁺; ¹H NMR (500 MHz, METHANOL-d₄) δ ppm: 1.50 (s, 3H), 1.54 (d, J=6.26 Hz, 6H), 1.85 (dd, J=4.50, 1.60 Hz, 2H), 2.09 (dd, J=4.58, 1.53 Hz, 2H), 2.40-2.30 (m, 1H), 2.40-2.50 (m, 1H), 3.75 (q, J=7.74 Hz, 1H), 3.95-4.00 (m, 3H), 4.00-4.05 (m, 1H), 4.13 (td, J=8.09, 5.04 Hz, 1H), 4.25 (t, J=8.01 Hz, 1H), 5.59 (q, J=6.22 Hz, 1H), 7.49 (s, 1H), 7.57 (d, J=7.63 Hz, 1H), 7.96 (t, J=7.71 Hz, 1H), 8.04-8.07 (m, 1H), 9.64 (s, 1H)

Example 494: 6-cyclopropoxy-N-(7-isopropoxy-2-(1-methyl-2-oxabicyclo[2.1.1]hexan-4-yl)imidazo[1,2-a]pyrimidin-6-yl)picolinamide; LCMS (ESI) m/z 450.1 (M+H)⁺; ¹H NMR (400 MHz, METHANOL-d₄) δ ppm: 0.83-0.93 (m, 4H), 1.47-1.50 (t, J=15.0 Hz, 9H), 1.87-1.89 (t, J=10.0 Hz, 2H), 2.12-2.13 (m, 2H), 4.01 (s, 2H), 4.42-4.46 (m, 1H), 5.58-5.64 (m, 1H), 7.06-7.08 (d, J=10.0 Hz, 1H), 7.61 (s, 1H), 7.85-7.94 (m, 2H), 9.77 (s, 1H)

Example 495: N-(7-isopropoxy-2-(1-methyl-2-oxabicyclo[2.1.1]hexan-4-yl)imidazo[1,2-a]pyrimidin-6-yl)pyrazolo[1,5-a]pyrimidine-7-carboxamide; LCMS (ESI) m/z 434.0 (M+H)⁺; ¹H NMR (500 MHz, METHANOL-d₄) δ ppm 1.52 (s, 3H), 1.60 (d, J=6.10 Hz, 6H), 1.86 (dd, J=4.58, 1.53 Hz, 2H), 2.03-2.15 (m, 2H), 4.00 (s, 2H), 5.49 (dt, J=12.36, 6.33 Hz, 1H), 6.93 (d, J=2.44 Hz, 1H), 7.41 (s, 1H), 7.79 (d, J=4.27 Hz, 1H), 8.36 (d, J=2.44 Hz, 1H), 8.72 (d, J=4.27 Hz, 1H), 9.67 (s, 1H)

Example 496: 1-(difluoromethyl)-N-(7-isopropoxy-2-(1-methyl-2-oxabicyclo[2.1.1]hexan-4-yl)imidazo[1,2-a]pyrimidin-6-yl)-2-oxo-1,2-dihydropyridine-3-carboxamide 2,2,2-trifluoroacetate; LCMS (ESI) m/z 460.0 (M+H)⁺; 1H NMR (500 MHz, METHANOL-d4) δ ppm: 1.53 (s, 3H) 1.60 (d, J=6.10 Hz, 6H) 1.98 (dd, J=4.88, 1.83 Hz, 2H) 2.20 (dd, J=4.88, 1.83 Hz, 2H) 4.04 (s, 2H) 5.50-5.74 (m, 1H) 6.81 (t, J=7.02 Hz, 1H) 7.81-8.15 (m, 2H) 8.19-8.25 (m, 1H) 8.72 (dd, J=7.33, 1.83 Hz, 1H) 9.94 (s, 1H), 12.24 (s, 1H)

Example 497: N-(7-isopropoxy-2-(1-methyl-2-oxabicyclo[2.1.1]hexan-4-yl)imidazo[1,2-a]pyrimidin-6-yl)-1-methyl-1H-pyrazole-3-carboxamide; LCMS (ESI) m/z 397.0 (M+H); ¹H NMR (500 MHz, DMSO-d₆) δ ppm: 1.42-1.52 (m, 8H), 1.69-1.85 (m, 2H), 2.02-2.18 (m, 2H), 3.85-3.93 (m, 2H), 3.99 (s, 3H), 5.32-5.47 (m, 1H), 6.83 (d, J=2.44 Hz, 1H), 7.38 (s, 1H), 7.94 (s, 2H), 9.25 (br s, 1H), 9.45-9.72 (m, 1H)

Example 498: 1-(difluoromethyl)-N-(7-isopropoxy-2-(1-methyl-2-oxabicyclo[2.1.1]hexan-4-yl)imidazo[1,2-a]pyrimidin-6-yl)-1H-pyrazole-3-carboxamide; LCMS (ESI) m/z 433.0 (M+H)⁺; ¹H NMR (500 MHz, DMSO-d₆) δ ppm: 1.40-1.50 (m, 11H), 1.71-1.85 (m, 2H), 1.97-2.13 (m, 2H), 3.90 (s, 2H), 5.32-5.49 (m, 1H), 7.07 (d, J=2.44 Hz, 1H), 7.80-8.23 (m, 2H), 8.49 (d, J=2.44 Hz, 1H), 9.38-9.68 (m, 2H)

Example 499: N-(7-isopropoxy-2-(1-methyl-2-oxabicyclo[2.1.1]hexan-4-yl)imidazo[1,2-a]pyrimidin-6-yl)-2-(trifluoromethyl)thiazole-4-carboxamide; LCMS (ESI) m/z 468.0 (M+H)⁺; ¹H NMR (500 MHz, METHANOL-d₄) δ 1.51-1.52 (m, 6H), 1.52-1.53 (m, 3H), 1.87 (d, J=4.5 Hz, 2H), 2.12 (d, J=4.5 Hz, 2H), 4.02 (s, 2H), 5.56 (t, J=6.0 Hz, 1H), 7.51-7.66 (m, 1H), 8.74-8.81 (m, 1H), 9.54 (s, 1H),

Example 500: N-(2-(3-oxabicyclo[3.1.0]hexan-6-yl)-7-isopropoxyimidazo[1,2-a]pyrimidin-6-yl)-1-methyl-1H-pyrazole-3-carboxamide Hydrochloride; LCMS (ESI) m/z 383.0 (M+H)⁺; ¹H NMR (500 MHz, METHANOL-d₄) δ: 1.56-1.57 (d, J=6.0 Hz, 6H), 1.96-1.97 (m, 1H), 2.19 (d, J=2 Hz, 2H), 3.79-3.81 (d, J=8.5 Hz, 2H), 4.02-4.04 (m, 5H), 5.59-5.64 (m, 1H), 6.88-6.89 (d, J=2.0 Hz, 2H), 7.67-7.74 (t, J=2.5 Hz, 1H), 9.70-9.74 (t, J=9.5 Hz, 1H),

Example 501: N-(2-(3-oxabicyclo[3.1.0]hexan-6-yl)-7-isopropoxyimidazo[1,2-a]pyrimidin-6-yl)-1-isopropyl-1H-pyrazole-3-carboxamide hydrochloride; LCMS (ESI) m/z 411.1 (M+H)⁺; 1H NMR (500 MHz, METHANOL-d₄) δ: 1.51-1.98 (m, 9H), 2.20-2.21 (d, J=3.0 Hz, 1H), 3.79-3.81 (d, J=8.5 Hz, 2H), 4.02-4.04 (d, J=9.0 Hz, 2H), 4.63-4.68 (m, 1H), 7.69 (s, 1H), 5.57-5.62 (m, 1H), 6.86-6.88 (d, J=12.0 Hz, 1H), 7.82-7.83 (d, J=2.0 Hz, 1H), 9.72 (s, 1H)

Example 502: N-(2-(3-oxabicyclo[3.1.0]hexan-6-yl)-7-isopropoxyimidazo[1,2-a]pyrimidin-6-yl)-1-(difluoromethyl)-1H-pyrazole-3-carboxamide Hydrochloride; LCMS (ESI) m/z 383.0 (M+H)⁺; ¹H NMR (500 MHz, METHANOL-d₄) δ ppm: 1.55 (d, J=6.0 Hz, 6H), 1.97 (t, J=3.5 Hz, 1H), 2.20 (d, J=3.5 Hz, 2H), 3.80 (d, J=8.5 Hz, 2H), 4.03 (d, J=8.5 Hz 2H), 5.61 (t, J=6.0 Hz, 1H), 7.08 (d, J=3.0 Hz, 1H), 7.80 (s, 1H), 8.27 (d, J=3.0 Hz, 1H), 9.72 (s, 1H)

Example 503: N-(2-(3-oxabicyclo[3.1.0]hexan-6-yl)-7-isopropoxyimidazo[1,2-a]pyrimidin-6-yl)-1-(2,2-difluoroethyl)-1H-pyrazole-3-carboxamide; LCMS (ESI) m/z 433.1 (M+H)⁺; ¹H NMR (400 MHz, METHANOL) δ ppm: 1.55-1.60 (m, 6H), 1.98 (t, J=3.6 Hz, 1H), 2.20-2.22 (m, 2H), 3.81 (d, J=8.8 Hz, 2H), 4.02-4.05 (m, 2H), 4.69-4.78 (m, 2H), 5.58-5.66 (m, 2H), 6.13-6.43 (m, 1H), 6.95 (d, J=2.4 Hz, 1H), 7.70 (s, 1H), 7.87-7.89 (m, 1H), 9.40 (s, 1H), 9.73 (s, 1H)

Example 504: N-(2-(3-oxabicyclo[3.1.0]hexan-6-yl)-7-isopropoxyimidazo[1,2-a]pyrimidin-6-yl)-2-isopropyloxazole-4-carboxamide; LCMS (ESI) m/z 412.0 (M+H)⁺; ¹H NMR (500 MHz, METHANOL-d₄) δ ppm: 1.41 (d, J=7.0 Hz, 6H), 1.57 (d, J=6.5 Hz, 6H), 1.97 (t, J=3.5 Hz, 1H), 2.21 (d, J=3.0 Hz, 2H), 3.18-3.21 (m, 1H), 3.80 (d, J=8.5 Hz, 2H), 4.03 (d, J=8.5 Hz, 2H), 5.60 (t, J=6.0 Hz, 1H), 7.69 (s, 1H), 8.52 (s, 1H), 9.73 (s, 1H).

Example 505: N-(7-cyclopropoxy-2-(1-methyl-2-oxabicyclo[2.1.1]hexan-4-yl)imidazo[1,2-a]pyrimidin-6-yl)-2-methoxynicotinamide 2,2,2-trifluoroacetate; LCMS (ESI) m/z 422.0 (M+H)⁺; ¹H NMR (500 MHz, METHANOL-d₄) δ ppm 1.05-1.15 (m, 4H), 1.54 (s, 3H), 1.99 (dd, J=4.27, 1.83 Hz, 2H), 2.21 (dd, J=4.88, 1.83 Hz, 2H), 4.05 (s, 2H), 4.27 (s, 3H), 4.70-4.77 (m, 1H), 7.26 (dd, J=7.33, 4.88 Hz, 1H), 7.91 (s, 1H), 8.45 (dd, J=4.88, 1.83 Hz, 1H), 8.57 (dd, J=7.33, 1.83 Hz, 1H), 9.99 (s, 1H)

Example 506: 6-(difluoromethyl)-N-(8-methoxy-2-((tetrahydro-2H-pyran-4-yl)methyl)imidazo[1,2-a]pyridin-6-yl)picolinamide; LCMS (ESI) m/z 417.2 (M+H)⁺; ¹H NMR (500 MHz, DMSO-d₆) δ ppm 1.15-1.33 (m, 4H), 1.56 (br dd, J=12.82, 1.83 Hz, 3H), 1.85-2.01 (m, 1H), 2.56 (d, J=6.71 Hz, 4H), 3.26 (br d, J=1.83 Hz, 2H), 3.82 (br dd, J=11.29, 2.75 Hz, 3H), 3.94 (s, 4H), 6.93-7.20 (m, 2H), 7.77 (s, 1H), 8.00 (t, J=4.27 Hz, 1H), 8.25-8.35 (m, 2H), 8.97 (d, J=1.83 Hz, 1H).

Example 507: 6-(difluoromethyl)-N-(8-methoxy-2-(6-oxaspiro[3.4]octan-2-yl)imidazo[1,2-a]pyridin-6-yl)picolinamide; LCMS (ESI) m/z 429.3 (M+H)⁺; ¹H NMR (500 MHz, DMSO-d₆) δ ppm 1.93-1.97 (m, 1H), 2.04-2.09 (m, 1H), 2.10-2.21 (m, 2H), 2.35-2.40 (m, 3H), 3.60-3.76 (m, 6H), 4.09-4.13 (m, 3H), 6.97-7.22 (m, 1H), 7.84 (br s, 1H), 8.05 (dd, J=6.71, 1.83 Hz, 1H), 8.31-8.39 (m, 2H), 9.34 (br s, 1H), 10.90 (br s, 1H).

Example 508: 6-(difluoromethyl)-N-(8-methoxy-2-(1-methyl-2-oxabicyclo[2.1.1]hexan-4-yl)imidazo[1,2-a]pyridin-6-yl)picolinamide 2,2,2-trifluoroacetate; ¹H NMR (500 MHz, DMSO-d₆) δ ppm 1.45 (s, 4H) 1.86 (br d, J=4.27 Hz, 2H) 2.16 (br d, J=3.66 Hz, 2H) 3.94 (s, 2H) 4.10 (s, 3H) 6.90-7.27 (m, 1H) 7.80 (br s, 1H) 8.05 (dd, J=6.41, 2.14 Hz, 1H) 8.28-8.40 (m, 2H) 9.32 (br s, 1H) 10.89 (br s, 1H)

Example 509: N-(2-(8-oxabicyclo[3.2.1]octan-3-yl)-8-methoxyimidazo[1,2-a]pyridin-6-yl)-6-(difluoromethyl)picolinamide 2,2,2-trifluoroacetate; LCMS (ESI) m/z 429.2 (M+H)⁺; ¹H NMR (500 MHz, DMSO-d₆) δ ppm 1.66-1.95 (m, 9H), 4.09 (s, 3H), 4.31 (br d, J=7.32 Hz, 1H), 4.42 (br s, 1H), 6.88-7.31 (m, 1H), 8.05 (dd, J=6.71, 2.44 Hz, 1H), 8.17-8.42 (m, 2H), 9.33 (br s, 1H), 10.88 (br s, 1H)

Example 510: 6-(difluoromethyl)-N-(2-(2,2-dimethyltetrahydro-2H-pyran-4-yl)-8-methoxyimidazo[1,2-a]pyridin-6-yl)picolinamide 2,2,2-trifluoroacetate; LCMS (ESI) m/z 431.3 (M+H)⁺; ¹H NMR (400 MHz, CHLOROFORM-d) δ ppm: 1.31 (d, J=9.29 Hz, 6H) 1.60 (t, J=12.93 Hz, 1H) 1.66-1.77 (m, 1H) 2.06 (br d, J=12.55 Hz, 2H) 3.42 (br t, J=12.55 Hz, 1H) 3.79-3.94 (m, 2H) 4.10 (s, 3H) 6.62-6.91 (m, 1H) 7.09 (s, 1H) 7.44 (s, 1H) 7.92 (d, J=7.78 Hz, 1H) 8.16 (t, J=7.78 Hz, 1H) 8.41 (d, J=7.78 Hz, 1H) 9.22 (s, 1H) 10.04 (s, 1H)

Example 511: N-(2-(3-cyanobicyclo[1.1.1]pentan-1-yl)-8-methoxyimidazo[1,2-a]pyridin-6-yl)-6-(difluoromethyl)picolinamide 2,2,2-trifluoroacetate; LCMS (ESI) m/z 410.2 (M+H)⁺; ¹H NMR (500 MHz, DMSO-d₆) δ ppm 2.66 (s, 5H), 4.05 (s, 3H), 6.89-7.30 (m, 1H), 7.65 (br s, 1H), 8.04 (dd, J=6.10, 3.05 Hz, 1H), 8.18-8.41 (m, 2H), 9.23 (br s, 1H), 10.80 (br s, 1H)

Example 512: 6-(difluoromethyl)-N-(2-(3-(difluoromethyl)bicyclo[1.1.1]pentan-1-yl)-8-methoxyimidazo[1,2-a]pyridin-6-yl)picolinamide 2,2,2-trifluoroacetate; LCMS (ESI) m/z 435.2 (M+H)⁺; ¹H NMR (500 MHz, DMSO-d₆) δ ppm 2.25 (s, 6H) 4.07 (s, 3H) 5.99-6.37 (m, 1H) 6.93-7.24 (m, 1H) 8.04 (dd, J=6.10, 2.44 Hz, 1H) 8.23-8.41 (m, 2H) 9.26 (br s, 1H) 10.84 (br s, 1H)

Example 513: N-(2-(2,2-difluorocyclopropyl)-8-methoxyimidazo[1,2-a]pyridin-6-yl)-6-(difluoromethyl)picolinamide, enantiomer 1; LCMS (ESI) m/z 395.0 (M+H)⁺; ¹H NMR (500 MHz, METHANOL-d₄) δ ppm: 1.90-2.00 (m, 2H), 2.85-2.95 (m, 1H), 4.05 (s, 3H), 6.75-7.00 (m, 1H), 7.05 (s, 1H), 7.72 (s, 1H), 7.94 (d, J=8.0 Hz, 1H), 8.22 (t, J=8.0 Hz, 1H), 8.35 (d, J=7.5 Hz, 1H), 8.95 (s, 1H)

Example 514: N-(2-(2,2-difluorocyclopropyl)-8-methoxyimidazo[1,2-a]pyridin-6-yl)-6-(difluoromethyl)picolinamide, enantiomer 2; LCMS (ESI) m/z 395.0 (M+H)⁺; ¹H NMR (500 MHz, METHANOL-d₄) δ ppm: 1.90-2.00 (m, 2H), 2.85-2.95 (m, 1H), 4.06 (s, 3H), 6.75-7.00 (m, 1H), 7.07 (s, 1H), 7.74 (s, 1H), 7.95 (d, J=8.0 Hz, 1H), 8.24 (t, J=8.0 Hz, 1H), 8.37 (d, J=7.5 Hz, 1H), 8.97 (s, 1H)

Example 515: 6-(difluoromethyl)-N-(7-methoxy-2-(1-methyl-2-oxabicyclo[2.1.1]hexan-4-yl)imidazo[1,2-a]pyridin-6-yl)picolinamide 2,2,2-trifluoroacetate; LCMS (ESI) m/z 415.0 (M+H)⁺; ¹H NMR (500 MHz, DMSO-d₆) δ ppm: 1.46 (s, 3H) 1.88 (dd, J=4.27, 1.83 Hz, 2H) 2.15 (d, J=4.27 Hz, 2H) 3.94 (s, 2H) 4.19 (s, 3H) 6.97-7.47 (m, 2H) 8.07 (dd, J=7.02, 1.53 Hz, 1H) 8.32-8.45 (m, 2H) 9.71 (s, 1H) 10.44 (s, 1H)

Example 516: N-(7-ethoxy-2-(tetrahydro-2H-pyran-4-yl)imidazo[1,2-a]pyridin-6-yl)-6-(2-methoxyethoxy)picolinamide; LCMS (ESI) m/z 441.1 (M+H)⁺; ¹H NMR (400 MHz, METHANOL-d₄) δ 1.39 (t, J=6.9 Hz, 3H), 1.60-1.87 (m, 2H), 1.95 (d, J=13.2 Hz, 2H), 2.76-2.91 (m, 1H), 3.43 (s, 3H), 3.56 (dd, J=12.1, 9.7, 2H), 3.73-3.64 (m, 2H), 3.90-4.12 (m, 4H), 4.29 (t, J=6.9, Hz, 2H), 6.54 (s, 1H), 6.77 (d, J=8.2 Hz, 1H), 7.26 (s, 1H), 7.56 (d, J=7.2 Hz, 1H), 7.60-7.73 (m, 1H), 9.19 (s, 1H)

Example 517: 6-(difluoromethyl)-N-(7-ethoxy-2-(1-methyl-2-oxabicyclo[2.2.2]octan-4-yl)imidazo[1,2-a]pyridin-6-yl)picolinamide 2,2,2-trifluoroacetate; LCMS (ESI) m/z 457.0 (M+H)⁺; ¹H NMR (500 MHz, DMSO-d₆) δ ppm 0.96 (s, 3H), 1.36-1.57 (m, 2H), 1.36-1.63 (m, 1H), 1.37-1.48 (m, 4H), 1.72-1.76 (m, 1H), 1.76-1.88 (m, 2H), 1.88-2.09 (m, 2H), 3.78 (s, 2H), 4.17-4.35 (m, 2H), 6.87-7.29 (m, 2H), 7.87-8.05 (m, 2H), 8.15-8.28 (m, 2H), 10.35-10.53 (m, 1H).

Example 518: N-(2-(3-oxabicyclo[3.1.0]hexan-1-yl)-7-ethoxyimidazo[1,2-a]pyridin-6-yl)-6-(difluoromethyl)picolinamide; LCMS (ESI) m/z 415.0 (M+H)⁺; ¹H NMR (500 MHz, DMSO-d₆) δ ppm 0.76-0.86 (m, 1H), 1.09 (dd, J=8.24, 4.58 Hz, 1H) 1.31 (t, J=7.02 Hz, 3H), 1.90 (br d, J=4.27 Hz, 1H), 3.56-3.73 (m, 3H), 3.80 (d, J=7.94 Hz, 1H), 4.11-4.23 (m, 2H), 6.74-7.06 (m, 2H), 7.82 (dd, J=6.71, 1.83 Hz, 1H), 7.95 (br s, 1H) 8.05-8.17 (m, 2H), 9.44 (br s, 1H), 10.38 (s, 1H)

Example 519: Cis 6-(difluoromethyl)-N-(7-ethoxy-2-(2-fluorocyclopropyl)imidazo[1,2-a]pyridin-6-yl)picolinamide hydrochloride; LCMS (ESI) m/z 391.1 (M+H); ¹H NMR: (400 MHz, METHANOL-d₄) δ ppm: 1.45-1.55 (m, 2H), 1.68 (t, J=7.2 Hz, 3H), 2.28-2.35 (m, 1H), 4.43-4.50 (m, 2H), 4.96-5.16 (m, 1H), 6.73-7.02 (m, 1H), 7.26 (s, 1H), 7.94-7.99 (m, 2H), 8.26-8.30 (m, 1H), 8.41 (d, J=8.0 Hz, 1H), 9.78 (s, 1H), 10.76 (s, 1H)

Example 520: N-(7-ethoxy-2-(3-methylbicyclo[1.1.1]pentan-1-yl)imidazo[1,2-a]pyridin-6-yl)-6-(trifluoromethyl)picolinamide 2,2,2-trifluoroacetate; LCMS (ESI) m/z 431.5 (M+H)⁺;

Example 521: N-(2-(3-cyanobicyclo[1.1.1]pentan-1-yl)-7-ethoxyimidazo[1,2-a]pyridin-6-yl)-6-(trifluoromethyl)picolinamide 2,2,2-trifluoroacetate; LCMS (ESI) m/z 442.5 (M+H)⁺;

Example 522: N-(2-(3-(difluoromethyl)bicyclo[1.1.1]pentan-1-yl)-7-ethoxyimidazo[1,2-a]pyridin-6-yl)-6-(trifluoromethyl)picolinamide 2,2,2-trifluoroacetate; LCMS (ESI) m/z 467.5 (M+H)⁺;

Example 523: N-(7-ethoxy-2-(3-(methoxymethyl)bicyclo[1.1.1]pentan-1-yl)imidazo[1,2-a]pyridin-6-yl)-6-(trifluoromethyl)picolinamide; LCMS (ESI) m/z 461.1 (M+H)⁺;

Example 524: N-(7-ethoxy-2-(3-methoxypropyl)imidazo[1,2-a]pyridin-6-yl)-6-methoxypicolinamide; LCMS (ESI) m/z 385.2 (M+H)⁺; ¹H NMR (400 MHz, CHLOROFORM-d) δ ppm: 1.52 (t, J=6.9 Hz, 3H), 2.16-1.95 (m, 2H), 2.78 (t, J=7.6 Hz, 2H), 3.34 (s, 3H), 3.44 (t, J=6.4 Hz, 2H), 4.05 (s, 3H), 4.16 (q, J=6.9 Hz, 2H), 6.85 (s, 1H), 6.95 (d, J=8.2 Hz, 1H), 7.21 (s, 1H), 7.77 (t, J=7.7 Hz, 1H), 7.85 (d, J=7.2 Hz, 1H), 9.45 (s, 1H), 10.40 (s, 1H)

Example 525: 6-(difluoromethyl)-N-(7-isopropoxy-2-(1-methyl-1H-pyrazol-5-yl)imidazo[1,2-a]pyridin-6-yl)picolinamide; LCMS (ESI) m/z 427.3 (M+H)⁺; ¹H NMR (500 MHz, DMSO-d₆) δ ppm 1.48 (d, J=5.49 Hz, 8H), 4.12 (s, 3H), 5.01-5.10 (m, 1H), 6.69 (br s, 1H), 6.94-7.29 (m, 1H), 7.34 (s, 1H), 7.52 (s, 1H), 8.05 (dd, J=7.02, 1.53 Hz, 1H), 8.33-8.40 (m, 2H), 8.51 (br s, 1H), 9.68 (br s, 1H), 10.64 (s, 1H)

Example 526: N-(2-cyclopropyl-7-isopropoxyimidazo[1,2-a]pyridin-6-yl)-6-(difluoromethyl)picolinamide; LCMS (ESI) m/z 387.2 (M+H)⁺; ¹H NMR (400 MHz, DMSO-d₆) δ ppm: 0.75-0.81 (m, 2H), 0.82-0.89 (m, 2H), 1.40 (d, J=6.0 Hz, 6H), 1.89-1.97 (m, 1H), 4.75-4.91 (m, 1H), 6.92-7.31 (m, 2H), 7.96-8.03 (m, 1H), 7.67 (s, 1H), 8.24-8.39 (m, 2H), 9.38 (s, 1H), 10.48 (s, 1H)

Example 527: 6-(difluoromethyl)-N-(7-isopropoxy-2-(1-methoxy-2-methylpropan-2-yl)imidazo[1,2-a]pyridin-6-yl)picolinamide; LCMS (ESI) m/z 433.4 (M+H)⁺; 1H NMR (400 MHz, METHANOL-d₄) δ ppm: 1.37 (s, 6H), 1.54 (d, J=6.02 Hz, 6H), 3.34 (s, 3H), 3.54 (s, 2H), 6.68-7.03 (m, 2H), 7.51 (s, 1H), 7.93 (d, J=7.78 Hz, 1H), 8.24 (t, J=7.78 Hz, 1H), 8.37 (dd, J=1.00, 7.78 Hz, 1H), 9.47 (s, 1H)

Example 528: N-(2-cyclopropyl-7-isopropoxyimidazo[1,2-a]pyridin-6-yl)-1-(difluoromethyl)-1H-pyrazole-3-carboxamide; LCMS (ESI) m/z 376.4 (M+H)⁺; ¹H NMR (400 MHz, DMSO-d₆) δ ppm: 0.74-0.80 (m, 2H), 0.82-0.89 (m, 2H), 1.36 (d, J=6.0 Hz, 6H), 1.87-1.98 (m, 1H), 4.71-4.85 (m, 1H), 7.03 (d, J=2.1 Hz, 2H), 7.64 (s, 1H), 7.79-8.11 (m, 1H) 8.46 (d, J=2.8 Hz, 1H), 9.14 (d, J=5.6 Hz, 1H), 9.34 (s, 1H)

Example 529: N-(7-isopropoxy-2-(1-methyl-2-oxabicyclo[2.1.1]hexan-4-yl)imidazo[1,2-a]pyridin-6-yl)-2-methyloxazole-4-carboxamide; LCMS (ESI) m/z 397.1 (M+H)⁺; ¹H NMR (400 MHz, METHANOL-d₄) δ ppm: 1.49-1.55 (m, 3H), 1.57 (d, J=6.27 Hz, 6H), 1.95-2.04 (m, 2H), 2.18-2.29 (m, 2H), 2.51-2.62 (m, 3H), 4.01-4.09 (m, 2H), 5.01-5.15 (m, 1H), 7.23-7.37 (m, 1H), 7.87-8.04 (m, 1H), 8.43-8.54 (m, 1H), 9.63-9.77 (m, 1H)

Example 530: 6-(difluoromethyl)-N-(7-isopropoxy-2-(tetrahydrofuran-3-yl)imidazo[1,2-a]pyridin-6-yl)picolinamide 2,2,2-trifluoroacetate; LCMS (ESI) m/z 417.0 (M+H)⁺; ¹H NMR (500 MHz, DMSO-d₆) δ ppm 1.48 (d, J=6.10 Hz, 7H), 1.83-1.95 (m, 1H), 2.02-2.11 (m, 1H), 2.19-2.31 (m, 1H), 2.32-2.40 (m, 1H), 3.89-3.97 (m, 3H) 4.97-5.09 (m, 1H) 6.94-7.36 (m, 1H), 7.64 (s, 1H) 8.07 (dd, J=7.02, 1.53 Hz, 1H), 8.29-8.50 (m, 3H), 9.78 (s, 1H), 10.65 (s, 1H)

Example 531: N-(2-(1,4-dioxan-2-yl)-7-isopropoxyimidazo[1,2-a]pyridin-6-yl)-6-(difluoromethyl)picolinamide; LCMS (ESI) m/z 433.2 (M+H)⁺; ¹H NMR (500 MHz, DMSO-d₆) δ ppm 1.40-1.45 (m, 7H), 3.55-3.62 (m, 1H), 3.72-3.79 (m, 2H), 3.84 (br d, J=2.44 Hz, 1H), 3.93-3.97 (m, 1H), 4.66 (dd, J=10.07, 2.75 Hz, 1H), 4.85-4.93 (m, 1H), 7.01-7.26 (m, 2H), 7.84 (s, 1H), 7.97-8.05 (m, 1H), 8.28-8.36 (m, 2H), 9.50 (s, 1H), 10.53 (s, 1H)

Example 532: 6-(difluoromethyl)-N-(7-isopropoxy-2-(1-methyl-2-oxabicyclo[2.1.1]hexan-4-yl)imidazo[1,2-a]pyridin-6-yl)picolinamide; LCMS (ESI) m/z 442.9 (M+H); ¹H NMR (500 MHz, METHANOL-d4) δ ppm 1.52 (s, 3H) 1.59 (d, J=6.10 Hz, 6H) 1.98 (dd, J=4.27, 1.83 Hz, 2H) 2.21 (dd, J=4.58, 1.53 Hz, 2H) 4.04 (s, 2H) 5.03-5.14 (m, 1H) 6.67-7.08 (m, 1H) 7.27 (s, 1H) 7.91-8.00 (m, 2H) 8.27 (t, J=7.94 Hz, 1H) 8.40 (d, J=7.94 Hz, 1H) 9.79 (s, 1H)

Example 533: 6-(difluoromethyl)-N-(7-isopropoxy-2-(1,3,3-trimethyl-2-oxabicyclo[2.1.1]hexan-4-yl)imidazo[1,2-a]pyridin-6-yl)picolinamide; LCMS (ESI) m/z 471.3 (M+H)⁺; ¹H NMR (400 MHz, CHLOROFORM-d) δ ppm: 1.41 (s, 6H), 1.46 (s, 3H), 1.51 (d, J=6.02 Hz, 6H), 1.85-2.00 (m, 2H), 2.36 (dd, J=1.63, 4.64 Hz, 2H), 4.70 (spt, J=5.90 Hz, 1H), 6.49-6.85 (m, 1H), 6.98 (s, 1H), 7.28 (s, 1H), 7.84 (d, J=7.78 Hz, 1H), 8.11 (t, J=7.78 Hz, 1H), 8.39 (d, J=7.78 Hz, 1H), 9.44 (s, 1H), 10.56 (s, 1H).

Example 534: 6-(difluoromethyl)-N-(7-isopropoxy-2-(1-methyl-2-oxabicyclo[2.2.1]heptan-4-yl)imidazo[1,2-a]pyridin-6-yl)picolinamide; LCMS (ESI) m/z 457.2 (M+H)⁺; ¹H NMR (400 MHz, CHLOROFORM-d) δ ppm: 1.48 (s, 3H) 1.51 (d, J=6.02 Hz, 6H), 1.75-2.28 (m, 6H), 3.96 (d, J=6.27 Hz, 1H), 4.10 (dd, J=3.26, 6.53 Hz, 1H), 4.70 (quin, J=6.02 Hz, 1H), 6.47-6.85 (m, 1H), 6.95 (s, 1H), 7.84 (d, J=7.28 Hz, 1H), 8.10 (t, J=7.78 Hz, 1H), 8.32-8.43 (m, 1H), 9.44 (s, 1H), 10.56 (s, 1H)

Example 535: 6-(difluoromethyl)-N-(2-(3-fluorobicyclo[1.1.1]pentan-1-yl)-8-methoxyimidazo[1,2-a]pyridin-6-yl)picolinamide; LCMS (ESI) m/z 403.1 (M+H)⁺; ¹H NMR (500 MHz, METHANOL-d₄) δ ppm: 2.57 (d, J=2.29 Hz, 6H), 4.19 (s, 3H), 6.80-7.03 (m, 1H), 7.70 (s, 1H), 7.98 (d, J=7.78 Hz, 1H), 8.12 (s, 1H), 8.26 (t, J=7.78 Hz, 1H), 8.40 (d, J=7.78 Hz, 1H), 9.34 (d, J=0.92 Hz, 1H),

Example 536: 6-(difluoromethyl)-N-(2-(3-(difluoromethyl)bicyclo[1.1.1]pentan-1-yl)-7-isopropoxyimidazo[1,2-a]pyridin-6-yl)picolinamide; LCMS (ESI) m/z 463.0 (M+H)⁺; ¹H NMR (500 MHz, DMSO-d₆) δ ppm 1.48 (d, J=6.10 Hz, 7H), 2.26 (s, 5H), 5.03-5.14 (m, 1H), 6.00-6.36 (m, 1H), 6.95-7.27 (m, 1H), 7.32 (s, 1H), 7.98-8.09 (m, 1H), 8.16 (br s, 1H), 8.31-8.43 (m, 2H), 9.71 (s, 1H), 10.64 (s, 1H)

Example 537: N-(2-(3-oxabicyclo[3.1.0]hexan-6-yl)-7-isopropoxyimidazo[1,2-a]pyridin-6-yl)-6-(difluoromethyl)picolinamide 2,2,2-trifluoroacetate; LCMS (ESI) m/z 429.0 (M+H)⁺; ¹H NMR (500 MHz, DMSO-d₆) δ ppm 1.46 (d, J=6.10 Hz, 6H), 1.95 (s, 1H), 2.17 (d, J=3.66 Hz, 2H), 3.71 (d, J=8.55 Hz, 2H), 3.93 (d, J=8.55 Hz, 2H), 5.02-5.14 (m, 1H), 6.98-7.28 (m, 1H), 7.34 (s, 1H), 8.01-8.15 (m, 2H), 8.27-8.42 (m, 2H), 9.69 (s, 1H), 10.61 (s, 1H)

Example 538: 6-(difluoromethyl)-N-(2-(3-fluorophenyl)-7-isopropoxyimidazo[1,2-a]pyridin-6-yl)picolinamide 2,2,2-trifluoroacetate; LCMS (ESI) m/z 441.3 (M+H)⁺; ¹H NMR (500 MHz, DMSO-d₆) δ ppm 1.49 (d, J=6.10 Hz, 9H), 4.99-5.15 (m, 2H), 7.04-7.31 (m, 2H), 7.30-7.37 (m, 2H), 7.55-7.65 (m, 1H), 7.68-7.77 (m, 3H), 8.04-8.07 (m, 1H), 8.34-8.39 (m, 2H), 8.68 (br s, 1H), 9.69 (s, 1H), 10.65 (s, 2H)

Example 539: N-(2-(3-fluorobicyclo[1.1.1]pentan-1-yl)-7-isopropoxyimidazo[1,2-a]pyridin-6-yl)-6-(trifluoromethyl)picolinamide 2,2,2-trifluoroacetate; LCMS (ESI) m/z 448.9 (M+H)⁺; ¹H NMR (500 MHz, DMSO-d₆) δ ppm 1.46 (d, J=6.10 Hz, 6H), 2.52 (br d, J=1.83 Hz, 3H), 5.06-5.22 (m, 1H), 7.34 (s, 1H) 8.30 (dd, J=7.32, 1.22 Hz, 1H), 8.41-8.60 (m, 2H), 9.73 (s, 1H), 10.55 (s, 1H)

Example 540: N-(7-isopropoxy-2-(1-methyl-2-oxabicyclo[2.1.1]hexan-4-yl)imidazo[1,2-a]pyridin-6-yl)-2-(2-methylpyridin-4-yl)oxazole-4-carboxamide 2,2,2-trifluoroacetate; LCMS (ESI) m/z 474.4 (M+H)⁺; ¹H NMR (400 MHz, METHANOL-d₄) δ ppm: 1.54 (s, 3H), 1.63 (d, J=6.02 Hz, 6H), 1.94-2.04 (m, 2H), 2.20-2.29 (m, 2H), 2.91 (s, 3H), 4.05-4.10 (m, 2H), 5.10-5.17 (m, 1H), 7.33-7.44 (m, 1H), 7.92-8.01 (m, 1H), 8.41 (dd, J=1.76, 6.02 Hz, 1H), 8.44-8.55 (m, 1H), 8.88-8.96 (m, 1H), 8.96-9.04 (m, 1H), 9.68 (s, 1H).

Example 541: N-(2-(bicyclo[1.1.1]pentan-1-yl)-7-isopropoxyimidazo[1,2-a]pyridin-6-yl)-2-(2-methylpyridin-4-yl)oxazole-4-carboxamide 2,2,2-trifluoroacetate; LCMS (ESI) m/z 444.4 (M+H)⁺; ¹H NMR (400 MHz, METHANOL-d₄) δ ppm: 1.62 (d, J=6.02 Hz, 6H), 2.25-2.36 (m, 6H), 2.63-2.70 (m, 1H), 2.90 (s, 3H), 5.09 (spt, J=6.11 Hz, 1H), 7.23-7.37 (m, 1H), 7.80-7.88 (m, 1H), 8.38 (dd, J=1.76, 6.02 Hz, 1H), 8.46 (d, J=1.51 Hz, 1H), 8.90 (d, J=6.02 Hz, 1H), 8.95-9.04 (m, 1H), 9.58-9.72 (m, 1H)

Example 542: N-(2-(3-(difluoromethyl)bicyclo[1.1.1]pentan-1-yl)-7-isopropoxyimidazo[1,2-a]pyridin-6-yl)-2-(2-methylpyridin-4-yl)oxazole-4-carboxamide 2,2,2-trifluoroacetate; LCMS (ESI) m/z 494.3 (M+H)⁺; ¹H NMR (400 MHz, METHANOL-d₄) δ 1.63 (d, J=6.02 Hz, 6H), 2.37 (s, 6H), 2.84 (s, 3H), 5.07-5.15 (m, 1H), 5.78-6.12 (m, 1H), 7.31 (s, 1H), 7.93 (s, 1H), 8.24 (dd, J=1.13, 5.90 Hz, 1H), 8.28-8.36 (m, 1H), 8.84 (d, J=5.77 Hz, 1H), 8.89-8.99 (m, 1H), 9.68 (s, 1H),

Example 543: N-(7-isopropoxy-2-(3-methoxycyclobutyl)imidazo[1,2-a]pyridin-6-yl)-2-(2-methylpyridin-4-yl)oxazole-4-carboxamide 2,2,2-trifluoroacetate; LCMS (ESI) m/z 462.4 (M+H)⁺; ¹H NMR (400 MHz, METHANOL-d4) δ ppm: 1.63 (d, J=6.02 Hz, 6H), 2.05 (s, 3H), 2.11-2.25 (m, 2H), 2.53-2.59 (m, 1H), 2.78 (s, 3H), 2.80-2.91 (m, 1H), 3.98-4.21 (m, 1H), 5.10 (td, J=6.02, 12.05 Hz, 1H), 7.27-7.35 (m, 1H), 7.84-8.00 (m, 3H), 8.11 (d, J=6.02 Hz, 1H), 8.20 (s, 1H), 8.78 (d, J=5.77 Hz, 1H), 8.87-8.94 (m, 1H), 9.65-9.71 (m, 1H)

Example 544: N-(2-(2-oxabicyclo[2.1.1]hexan-4-yl)-8-methoxyimidazo[1,2-a]pyridin-6-yl)-6-(difluoromethyl)picolinamide 2,2,2-trifluoroacetate; LCMS (ESI) m/z 401.2 (M+H)⁺; ¹H NMR (400 MHz, METHANOL-d₄) δ ppm 2.03 (dd, J=4.64, 1.13 Hz, 2H), 2.38-2.43 (m, 2H), 4.04 (s, 2H), 4.18-4.24 (m, 3H), 4.69-4.75 (m, 1H), 6.79-7.08 (m, 1H), 7.85 (s, 1H), 8.00 (d, J=7.78 Hz, 1H), 8.20-8.22 (m, 1H), 8.27 (t, J=7.91 Hz, 1H), 8.41 (d, J=7.78 Hz, 1H), 9.40 (d, J=1.00 Hz, 1H), 9.38-9.42 (m, 1H)

Example 545: N-(8-chloro-7-ethoxy-2-(tetrahydro-2H-pyran-4-yl)imidazo[1,2-a]pyridin-6-yl)-6-(difluoromethyl)picolinamide; LCMS (ESI) m/z 480.9 (M+H)⁺

Example 546: N-(8-fluoro-7-isopropoxy-2-(1-methyl-2-oxabicyclo[2.1.1]hexan-4-yl)imidazo[1,2-a]pyridin-6-yl)-1-isopropyl-1H-pyrazole-3-carboxamide 2,2,2-trifluoroacetate; LCMS (ESI) m/z 442.0 (M+H)⁺; ¹H NMR (500 MHz, DMSO-d₆) δ ppm 1.40-1.46 (m, 8H), 1.50 (d, J=6.71 Hz, 6H), 1.79 (dd, J=4.27, 1.22 Hz, 2H), 2.04 (br d, J=3.66 Hz, 2H), 3.90 (s, 2H), 4.66 (dt, J=12.97, 6.64 Hz, 1H), 4.72-4.81 (m, 1H), 6.81 (d, J=2.44 Hz, 1H), 8.02 (d, J=2.44 Hz, 1H), 8.05-8.15 (m, 1H), 9.28 (br s, 1H), 9.32-9.46 (m, 1H)

Example 547: 1-(difluoromethyl)-N-(8-fluoro-7-isopropoxy-2-(1-methyl-2-oxabicyclo[2.1.1]hexan-4-yl)imidazo[1,2-a]pyridin-6-yl)-1H-pyrazole-3-carboxamide; LCMS (ESI) m/z 450.0 (M+H); ¹H NMR (500 MHz, DMSO-d₆) δ ppm 1.38 (d, J=6.10 Hz, 6H), 1.44 (s, 3H), 1.80 (dd, J=4.58, 1.53 Hz, 2H), 2.05 (br d, J=3.05 Hz, 2H), 3.91 (s, 2H), 4.69 (br s, 1H), 7.07 (d, J=3.05 Hz, 1H), 7.79-8.18 (m, 2H), 8.48 (d, J=3.05 Hz, 1H), 9.15 (s, 1H), 9.55 (s, 1H)

Example 548: N-(8-fluoro-7-isopropoxy-2-(1-methyl-2-oxabicyclo[2.1.1]hexan-4-yl)imidazo[1,2-a]pyridin-6-yl)pyrazolo[1,5-a]pyridine-7-carboxamide 2,2,2-trifluoroacetate; LCMS (ESI) m/z 450.0 (M+H)⁺; ¹H NMR (500 MHz, DMSO-d₆) δ ppm 1.45 (s, 3H), 1.49 (d, J=6.10 Hz, 6H), 1.81 (dd, J=4.27, 1.22 Hz, 2H), 2.07 (br d, J=3.05 Hz, 2H), 3.92 (s, 2H), 4.88-5.09 (m, 1H), 7.02 (d, J=2.44 Hz, 1H), 7.53 (dd, J=8.85, 7.02 Hz, 1H), 8.02 (dd, J=7.32, 1.22 Hz, 1H), 8.14 (dd, J=8.55, 1.22 Hz, 2H), 8.36 (d, J=2.44 Hz, 1H), 9.67 (s, 1H), 13.34 (br s, 1H)

Example 549: 6-(difluoromethyl)-N-(8-methoxy-2-(tetrahydro-2H-pyran-3-yl)imidazo[1,2-a]pyrazin-6-yl)picolinamide; LCMS (ESI) m/z 404.2 (M+H)⁺; ¹H NMR (500 MHz, DMSO-d₆) δ ppm 1.63-1.70 (m, 2H), 1.75-1.86 (m, 1H), 2.02-2.13 (m, 1H), 2.97 (tt, J=10.30, 4.04 Hz, 1H), 3.40-3.59 (m, 4H), 3.81-3.89 (m, 1H), 3.99-4.04 (m, 1H), 4.10 (s, 3H), 7.06-7.30 (m, 1H), 7.06-7.30 (m, 1H), 8.03 (dd, J=7.02, 1.53 Hz, 1H), 8.10 (s, 1H), 8.31-8.37 (m, 2H), 9.02 (s, 1H), 9.99 (s, 1H)

Example 550: N-(2-(3-oxabicyclo[3.1.0]hexan-1-yl)-8-methoxyimidazo[1,2-a]pyrazin-6-yl)-6-(difluoromethyl)picolinamide 2,2,2-trifluoroacetate; ¹H NMR (500 MHz, DMSO-d₆) δ ppm 0.83 (t, J=4.58 Hz, 1H), 1.20 (dd, J=7.94, 4.27 Hz, 1H), 1.85 (ddd, J=7.94, 4.88, 3.05 Hz, 1H), 3.64-3.73 (m, 2H), 3.88-3.94 (m, 2H), 3.98 (s, 2H), 6.94-7.17 (m, 1H), 7.91 (dd, J=7.32, 1.83 Hz, 1H), 8.00 (s, 1H), 8.18-8.25 (m, 2H), 8.86 (s, 1H), 9.86 (s, 1H).

Example 551: N-(2-(3-oxabicyclo[3.1.0]hexan-6-yl)-8-methoxyimidazo[1,2-a]pyrazin-6-yl)-6-(difluoromethyl)picolinamide 2,2,2-trifluoroactetate; LCMS (ESI) m/z 402.2 (M+H)⁺; ¹H NMR (500 MHz, DMSO-d₆) δ ppm 1.90 (t, J=3.36 Hz, 1H), 2.09 (d, J=3.66 Hz, 2H), 3.71 (d, J=8.55 Hz, 2H), 3.91 (d, J=8.55 Hz, 2H), 4.09 (s, 3H), 7.07-7.30 (m, 1H), 8.00-8.09 (m, 2H), 8.29-8.36 (m, 2H), 8.99 (s, 1H), 9.97 (s, 1H)

Example 552: 6-(difluoromethyl)-N-(2-(3-(difluoromethyl)bicyclo[1.1.1]pentan-1-yl)-8-methoxyimidazo[1,2-a]pyrazin-6-yl)picolinamide 2,2,2-trifluoroacetate; LCMS (ESI) m/z 436.2 (M+H)⁺; ¹H NMR (500 MHz, DMSO-d₆) δ ppm 2.16 (s, 6H), 4.10 (s, 3H), 6.01-6.27 (m, 1H), 7.03-7.32 (m, 1H), 8.03 (dd, J=7.02, 2.14 Hz, 1H), 8.11 (s, 1H) 8.31-8.35 (m, 1H), 9.01 (s, 1H), 9.98 (s, 1H)

Example 553: 6-(difluoromethyl)-N-(8-methoxy-2-(3-methoxybicyclo[1.1.1]pentan-1-yl)imidazo[1,2-a]pyrazin-6-yl)picolinamide 2,2,2-trifluoroacetate; LCMS (ESI) m/z 416.2 (M+H)⁺; ¹H NMR (400 MHz, CHLOROFORM-d) δ ppm 2.40-2.46 (m, 6H) 3.38-3.41 (m, 3H) 4.28 (s, 3H) 6.65-6.96 (m, 1H) 7.59 (s, 1H) 7.92-7.98 (m, 1H) 8.18 (t, J=7.78 Hz, 1H) 8.42-8.45 (m, 1H) 9.15 (s, 1H) 10.14 (s, 1H) 12.43-12.49 (m, 1H)

Example 554: 6-(difluoromethyl)-N-(8-methoxy-2-(1-methyl-2-oxabicyclo[2.1.1]hexan-4-yl)imidazo[1,2-a]pyrazin-6-yl)picolinamide 2,2,2-trifluoroacetate; LCMS (ESI) m/z 416.2 (M+H)⁺; ¹H NMR (400 MHz, CHLOROFORM-d) δ ppm 1.56 (s, 3H) 2.03-2.10 (m, 2H) 2.25-2.31 (m, 2H) 4.11 (s, 2H) 4.26 (s, 3H) 6.64-6.94 (m, 1H) 7.59 (s, 1H) 7.93 (dd, J=7.78, 0.75 Hz, 1H) 8.16 (t, J=7.91 Hz, 1H) 8.42 (dd, J=7.78, 0.75 Hz, 1H) 9.14 (s, 1H) 10.11 (s, 1H)

Example 555: 6-(difluoromethyl)-N-(8-methoxy-2-(1-methyl-2-oxabicyclo[3.1.1]heptan-5-yl)imidazo[1,2-a]pyrazin-6-yl)picolinamide; LCMS (ESI) m/z 430.3 (M+H)⁺;

Example 556: N-(2-(3-oxabicyclo[4.1.0]heptan-7-yl)-8-methoxyimidazo[1,2-a]pyrazin-6-yl)-6-(difluoromethyl)picolinamide 2,2,2-trifluoroacetate; LCMS (ESI) m/z 416.3 (M+H)⁺; ¹H NMR (400 MHz, CHLOROFORM-d) δ ppm 1.78-1.86 (m, 1H), 1.91-2.06 (m, 1H), 1.93-2.04 (m, 2H), 2.19 (t, J=4.77 Hz, 1H), 3.27-3.39 (m, 1H), 3.73 (ddd, J=11.61, 6.21, 2.76 Hz, 1H), 3.94 (dd, J=11.80, 3.51 Hz, 1H), 4.09 (d, J=11.55 Hz, 1H), 4.24 (s, 3H), 6.65-6.97 (m, 1H), 7.52 (s, 1H), 7.94 (d, J=8.03 Hz, 1H), 8.17 (t, J=7.91 Hz, 1H), 8.44 (dd, J=7.78, 0.75 Hz, 1H), 9.07 (s, 1H), 10.06 (s, 1H)

Example 557: 6-(difluoromethyl)-N-(8-methoxy-2-(6-oxaspiro[3.4]octan-2-yl)imidazo[1,2-a]pyrazin-6-yl)picolinamide 2,2,2-trifluoroacetate; LCMS (ESI) m/z 430.3 (M+H)⁺; ¹H NMR (400 MHz, CHLOROFORM-d) δ ppm 2.04 (t, J=7.15 Hz, 1H), 2.19 (t, J=6.78 Hz, 1H), 2.40-2.50 (m, 1H), 2.52-2.65 (m, 3H), 3.78 (s, 1H), 3.85-4.00 (m, 4H), 4.27 (d, J=0.75 Hz, 3H), 6.65-6.94 (m, 1H), 7.55-7.62 (m, 1H), 7.94 (d, J=7.78 Hz, 1H), 8.17 (t, J=7.91 Hz, 1H), 8.42 (d, J=7.78 Hz, 1H), 9.17 (d, J=3.01 Hz, 1H), 10.14 (s, 1H)

Example 558: 6-(difluoromethyl)-N-(8-methoxy-2-(6-oxaspiro[2.5]octan-1-yl)imidazo[1,2-a]pyrazin-6-yl)picolinamide 2,2,2-trifluoroacetate; LCMS (ESI) m/z 430.3 (M+H)⁺; ¹H NMR (500 MHz, DMSO-d₆) δ ppm 0.98 (dd, J=8.55, 4.27 Hz, 1H) 1.11 (t, J=4.88 Hz, 1H) 1.33 (ddd, J=10.07, 6.71, 3.36 Hz, 1H) 1.55 (br s, 1H) 1.46-1.52 (m, 2H) 2.04 (dd, J=8.55, 5.49 Hz, 1H) 3.62-3.71 (m, 2H) 4.10 (s, 3H) 6.99-7.30 (m, 3H) 8.04 (dd, J=7.32, 1.22 Hz, 1H) 8.11 (s, 1H) 8.31-8.37 (m, 2H) 9.00 (s, 1H) 9.99 (s, 1H)

Example 559: 6-(difluoromethyl)-N-(2-(1-(fluoromethyl)-2-oxabicyclo[2.1.1]hexan-4-yl)-8-methoxyimidazo[1,2-a]pyrazin-6-yl)picolinamide; LCMS (ESI) m/z 434.2 (M+H)⁺; ¹H NMR (500 MHz, DMSO-d₆) δ ppm 1.88 (dd, J=4.27, 1.22 Hz, 2H) 2.14-2.19 (m, 2H) 3.99 (s, 2H) 4.11 (s, 3H) 4.65-4.77 (m, 3H) 7.06-7.29 (m, 1H) 8.00-8.06 (m, 1H) 8.20 (s, 1H) 8.30-8.35 (m, 2H) 9.02 (s, 1H) 9.99 (s, 1H)

Example 560: 6-(difluoromethyl)-N-(2-(2,2-dimethyltetrahydro-2H-pyran-4-yl)-8-methoxyimidazo[1,2-a]pyrazin-6-yl)picolinamide 2,2,2-trifluoroacetate; LCMS (ESI) m/z 423.3 (M+H)⁺; ¹H NMR (500 MHz, DMSO-d₆) δ ppm 1.17-1.20 (m, 4H), 1.27 (s, 4H), 1.47-1.59 (m, 2H), 1.84-1.91 (m, 2H), 3.15 (tt, J=12.51, 3.66 Hz, 1H), 3.70-3.75 (m, 2H), 4.11 (s, 3H), 7.06-7.31 (m, 1H), 8.02-8.10 (m, 2H), 8.30-8.37 (m, 2H), 9.03 (s, 1H), 10.00 (s, 1H)

Example 561; N-(2-(2-oxabicyclo[2.2.2]octan-4-yl)-8-methoxyimidazo[1,2-a]pyrazin-6-yl)-6-(difluoromethyl)picolinamide; LCMS (ESI) m/z 444.3 (M+H)⁺; ¹H NMR (400 MHz, CHLOROFORM-d) δ ppm 1.21 (s, 2H), 1.76-1.86 (m, 2H), 2.03 (td, J=11.17, 5.27 Hz, 2H), 2.08-2.24 (m, 1H), 2.17 (td, J=11.55, 5.02 Hz, 2H), 4.12-4.16 (m, 2H) 4.24 (s, 2H), 6.63-6.93 (m, 1H), 7.47 (s, 1H), 7.93 (d, J=7.03 Hz, 1H), 8.15 (t, J=7.78 Hz, 1H), 8.41 (d, J=7.78 Hz, 1H), 9.11 (s, 1H), 10.07 (s, 1H) Example 562: N-(2-cyclopropyl-8-methoxyimidazo[1,2-a]pyrazin-6-yl)-6-(difluoromethyl)picolinamide 2,2,2-trifluoroacetate; LCMS (ESI) m/z 360.2 (M+H)⁺; ¹H NMR (500 MHz, DMSO-d₆) δ ppm 0.81-0.85 (m, 2H), 0.92-0.97 (m, 2H), 2.02-2.06 (m, 1H), 4.08 (s, 3H), 6.98-7.29 (m, 2H), 8.01-8.05 (m, 2H), 8.29-8.36 (m, 2H), 8.97 (s, 1H), 9.95 (s, 1H).

Example 563: 6-(difluoromethyl)-N-(8-methoxy-2-(2-(trifluoromethoxy)ethyl)imidazo[1,2-a]pyrazin-6-yl)picolinamide 2,2,2-trifluoroacetate; LCMS (ESI) m/z 432.3 (M+H)⁺; ¹H NMR (400 MHz, CHLOROFORM-d) δ ppm 3.41 (t, J=5.65 Hz, 2H), 4.29-4.32 (m, 3H), 4.35-4.42 (m, 2H), 6.67-6.97 (m, 1H), 7.68-7.74 (m, 1H), 7.96 (d, J=7.03 Hz, 1H), 8.19 (t, J=7.91 Hz, 1H), 8.45 (d, J=7.78 Hz, 1H), 9.17-9.22 (m, 1H), 10.14 (s, 1H)

Example 564: 6-(difluoromethyl)-N-(7-methoxy-2-(3-methoxycyclobutyl)imidazo[1,2-a]pyrimidin-6-yl)picolinamide; LCMS (ESI) m/z 404.0 (M+H)⁺; ¹H NMR (500 MHz, DMSO-d₆) δ ppm 2.02-2.13 (m, 2H) 2.32-2.44 (m, 2H) 2.62-2.72 (m, 1H) 3.16-3.22 (m, 2H) 3.86-3.96 (m, 1H) 4.21 (s, 3H) 6.94-7.34 (m, 1H) 7.90-8.02 (m, 1H) 8.08 (dd, J=6.71, 1.83 Hz, 1H) 8.27-8.43 (m, 2H) 9.71-9.91 (m, 1H) 10.16-10.33 (m, 1H)

Example 565: 6-(difluoromethyl)-N-(7-methoxy-2-(3-methoxybicyclo[1.1.1]pentan-1-yl)imidazo[1,2-a]pyrimidin-6-yl)picolinamide; ¹H NMR (500 MHz, DMSO-d₆) δ ppm 2.24 (s, 6H), 3.28 (s, 1H) 4.20 (s, 3H) 7.01-7.31 (m, 1H) 7.92 (br s, 1H) 8.08 (dd, J=6.71, 2.44 Hz, 1H), 8.26-8.42 (m, 2H) 10.26 (s, 1H)

Example 566: 6-(difluoromethyl)-N-(2-(3-fluorobicyclo[1.1.1]pentan-1-yl)-7-methoxyimidazo[1,2-a]pyrimidin-6-yl)picolinamide 2,2,2-trifluoroacetate; ¹H NMR (500 MHz, DMSO-d₆) δ ppm 2.37-2.46 (m, 5H), 4.15 (s, 3H), 6.98-7.37 (m, 1H), 7.88 (br s, 1H), 8.05 (dd, J=6.41, 2.75 Hz, 1H), 8.20-8.39 (m, 2H). 9.67-9.76 (m, 1H), 10.21 (s, 1H)

Example 567: 6-(difluoromethyl)-N-(7-methoxy-2-(1-methyl-2-oxabicyclo[2.1.1]hexan-4-yl)imidazo[1,2-a]pyrimidin-6-yl)picolinamide 2,2,2-trifluoroacetate; ¹H NMR (500 MHz, METHANOL-d₄) δ ppm 1.52 (s, 3H), 1.91-2.05 (m, 2H), 2.13-2.24 (m, 2H), 4.03 (s, 2H), 4.37 (s, 3H), 6.68-7.15 (m, 1H), 7.89 (s, 1H) 8.00 (d, J=7.94 Hz, 1H), 8.28 (t, J=7.63 Hz, 1H), 8.41 (d, J=7.94 Hz, 1H), 9.93 (s, 1H)

Example 568: N-(2-(3-oxabicyclo[3.1.0]hexan-6-yl)-7-methoxyimidazo[1,2-a]pyrimidin-6-yl)-6-(difluoromethyl)picolinamide 2,2,2-trifluoroacetate; LCMS (ESI) m/z 402.0 (M+H)⁺; ¹H NMR (500 MHz, DMSO-d₆) δ ppm 1.90 (br s, 1H), 2.15 (br s, 2H), 3.72 (d, J=7.94 Hz, 2H), 3.92 (d, J=8.55 Hz, 2H), 4.17 (s, 3H), 6.97-7.34 (m, 1H), 7.83 (br s, 1H), 8.01-8.13 (m, 1H), 8.30-8.42 (m, 2H), 10.13-10.31 (m, 1H).

Example 569: 6-(difluoromethyl)-N-(7-methoxy-2-(tetrahydro-2H-pyran-3-yl)imidazo[1,2-a]pyrimidin-6-yl)picolinamide; ¹H NMR (500 MHz, DMSO-d₆) δ ppm: 1.58-1.86 (m, 4H) 2.01-2.15 (m, 1H) 3.01 (br s, 1H) 3.24-3.60 (m, 1H) 3.84 (dt, J=10.99, 3.66 Hz, 1H) 3.97-4.06 (m, 1H) 4.19 (s, 3H) 6.98-7.50 (m, 1H) 7.92 (br s, 1H)

Example 570: N-(7-ethoxy-2-(tetrahydro-2H-pyran-4-yl)imidazo[1,2-a]pyrimidin-6-yl)-6,7-dihydro-5H-cyclopenta[b]pyridine-2-carboxamide hydrochloride; LCMS (ESI) m/z 408.2 (M+H)⁺; ¹H NMR (400 MHz, METHANOL-d₄) δ: 1.62 (t, J=7.2 Hz, 3H), 1.79-1.90 (m, 2H), 2.02 (d, J=12.4 Hz, 2H), 2.20-2.27 (m, 2H), 3.05-3.18 (m, 5H), 3.57-3.64 (m, 2H), 4.07 (dd, J=11.6 Hz, J=2.8 Hz, 2H), 4.80 (q, J=7.2 Hz, 2H), 7.78-7.86 (m, 2H), 8.03 (d, J=7.6 Hz, 1H), 9.90 (s, 1H)

Example 571: 6-(difluoromethyl)-N-(7-isopropoxy-2-(tetrahydro-2H-pyran-3-yl)imidazo[1,2-a]pyrimidin-6-yl)picolinamide 2,2,2-trifluoroacetate; LCMS (ESI) m/z 432.0 (M+H)⁺; ¹H NMR (500 MHz, DMSO-d₆) δ ppm 1.50 (d, J=6.10 Hz, 8H), 1.60-1.92 (m, 4H), 2.03-2.17 (m, 2H), 3.84 (br d, J=10.99 Hz, 2H), 3.95-4.09 (m, 1H), 5.34-5.56 (m, 1H), 7.06 (s, 1H), 7.17 (s, 1H), 7.28 (s, 1H) 7.90 (br s, 1H), 8.06 (s, 1H), 8.25-8.38 (m, 2H). 9.79 (br s, 1H), 10.40 (s, 1H)

Example 572: N-(2-(3-oxabicyclo[3.1.0]hexan-6-yl)-7-isopropoxyimidazo[1,2-a]pyrimidin-6-yl)-6-(difluoromethyl)picolinamide 2,2,2-trifluoroacetate; LCMS (ESI) m/z 430.0 (M+H)⁺; ¹H NMR (500 MHz, DMSO-d₆) δ ppm 1.50 (d, J=6.71 Hz, 6H), 3.59-3.67 (m, 1H), 3.71 (d, J=8.55 Hz, 2H), 3.82 (d, J=8.55 Hz, 1H), 3.93 (d, J=8.55 Hz, 2H), 4.03 (s, 2H), 5.38-5.51 (m, 1H), 7.06 (s, 1H), 7.17 (s, 1H), 7.28 (s, 1H), 7.88 (s, 1H) 7.98-8.10 (m, 1H), 8.24-8.48 (m, 2H), 9.79 (s, 1H), 10.40 (s, 1H)

Example 573: N-(2-(bicyclo[1.1.1]pentan-1-yl)-7-isopropoxyimidazo[1,2-a]pyrimidin-6-yl)-6-(difluoromethyl)picolinamide 2,2,2-trifluoroacetate; LCMS (ESI) m/z 414.0 (M+H)⁺; ¹H NMR (500 MHz, DMSO-d6) δ ppm 1.50 (d, J=6.10 Hz, 6H) 2.17 (s, 5H) 5.35-5.52 (m, 1H) 7.06-7.31 (m, 1H) 7.79-7.97 (m, 1H) 8.03-8.17 (m, 1H) 8.24-8.44 (m, 2H) 9.79 (br s, 1H) 10.41 (br s, 1H)

Example 574: 6-(difluoromethyl)-N-(2-(1-methyl-2-oxabicyclo[2.1.1]hexan-4-yl)-8-propoxyimidazo[1,2-a]pyrazin-6-yl)picolinamide; LCMS (ESI) m/z 444.2 (M+H)⁺; ¹H NMR (400 MHz, METHANOL-d₄) δ ppm: 1.14 (t, J=7.40 Hz, 3H), 1.51 (s, 3H), 9.01 (s, 1H), 1.82-2.03 (m, 4H), 2.10-2.24 (m, 2H), 4.04 (s, 2H), 4.44-4.72 (m, 2H), 6.70-7.08 (m, 1H), 7.89 (s, 1H), 7.95 (d, J=7.03 Hz, 1H), 8.24 (t, J=7.78 Hz, 1H), 8.31-8.44 (m, 1H)

Example 575: N-(3-chloro-7-isopropoxy-2-(1-methyl-2-oxabicyclo[2.1.1]hexan-4-yl)imidazo[1,2-a]pyridin-6-yl)-6-(difluoromethyl)picolinamide 2,2,2-trifluoroacetate; LCMS (ESI) m/z 477.3 (M+H); ¹H NMR (500 MHz, DMSO-d₆) δ ppm 1.40-1.49 (m, 9H) 1.80-1.95 (m, 2H) 2.15 (dd, J=4.27, 1.83 Hz, 2H) 3.97 (s, 2H) 4.95 (quin, J=6.10 Hz, 1H) 7.04-7.27 (m, 1H) 7.33 (s, 1H) 8.01-8.08 (m, 1H) 8.35 (d, J=4.88 Hz, 1H) 9.32 (s, 1H) 10.64 (s, 1H)

Example 576: N-(3-chloro-7-isopropoxy-2-(1-methyl-2-oxabicyclo[2.1.1]hexan-4-yl)imidazo[1,2-a]pyrimidine-6-yl)-6-(difluoromethyl)picolinamide 2,2,2-trifluoroacetate; LCMS (ESI) m/z 478.3 (M+H); ¹H NMR (600 MHz, DMSO-d₆) δ ppm 1.45 (s, 3H), 1.48 (d, J=6.60 Hz, 7H), 1.85 (dd, J=4.04, 1.83 Hz, 2H), 2.14 (dd, J=4.40, 1.47 Hz, 2H), 3.95 (s, 2H), 5.35-5.45 (m, 1H), 7.06-7.28 (m, 1H), 8.02-8.08 (m, 1H), 8.34-8.37 (m, 2H), 9.34 (s, 1H), 10.40 (s, 1H).

### Example 577: N-(8-fluoro-7-isopropoxy-2-(1-methyl-2-oxabicyclo[2.1.1]hexan-4-yl)imidazo[1,2-a]pyridin-6-yl)pyrazolo[1,5-a]pyrimidine-3-carboxamide

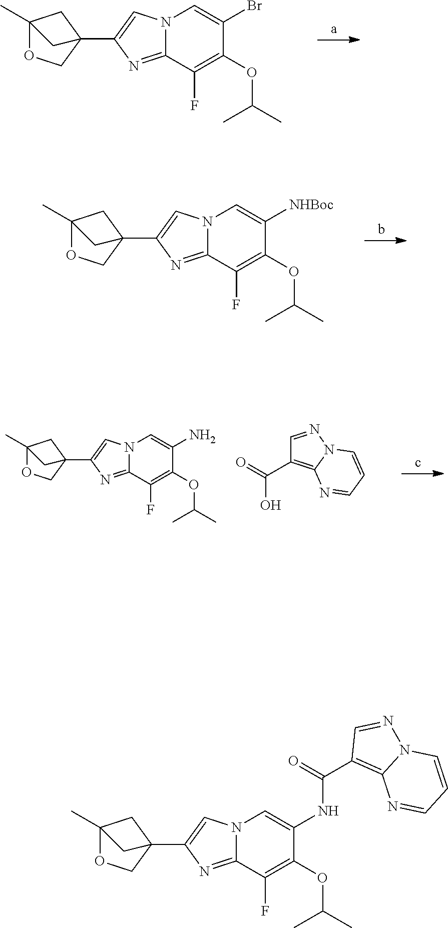

Step a: to a solution of 6-bromo-8-fluoro-7-isopropoxy-2-(1-methyl-2-oxabicyclo[2.1.1]hexan-4-yl)imidazo[1,2-a]pyridine (see Example 322 step d.) (120 mg, 325 μmol), K₂CO₃ (89 mg, 650 μmol), tBu-BrettPhos Pd G3 (14.73 mg, 16.25 μmol) were placed under an atmosphere of N2, and tBuOH (325.00 uL) was added. The vial was sealed, and the solution was heated to 70° C. overnight. Then, MeOH and silica was added, and the solvent was removed. Purification via column chromotography (5-100% EtOAc) gave tert-butyl N-[8-fluoro-7-isopropoxy-2-(1-methyl-2-oxabicyclo[2.1.1]hexan-4-yl)imidazo[1,2-a]pyridin-6-yl]carbamate (90 mg, 221 μmol, 68% yield). LCMS (ESI) m/z 406.0 (M+H)⁺; 1H NMR (400 MHz, CHLOROFORM-d) δ ppm 1.33 (dd, J=6.15, 0.88 Hz, 5H) 1.38 (s, 11H) 1.44-1.48 (m, 5H) 1.85-1.92 (m, 2H) 1.94-2.07 (m, 3H) 3.95-4.03 (m, 2H) 4.59-4.68 (m, 1H) 7.26 (d, J=3.26 Hz, 1H) 8.01 (d, J=1.76 Hz, 1H).

Step b: tert-butyl N-[8-fluoro-7-isopropoxy-2-(1-methyl-2-oxabicyclo[2.1.1]hexan-4-yl)imidazo[1,2-a]pyridin-6-yl]carbamate (90 mg, 221 μmol) was dissolved in HCl (4 M, 1.39 mL) and stirred at rt. The solution was then concentrated to give 8-fluoro-7-isopropoxy-2-(1-methyl-2-oxabicyclo[2.1.1]hexan-4-yl)imidazo[1,2-a]pyridin-6-amine, which was used without any further purification assuming quantitative yield. LCMS (ESI) m/z 306.0 (M+H)⁺.

Step c: To a mixture of 8-fluoro-7-isopropoxy-2-(1-methyl-2-oxabicyclo[2.1.1]hexan-4-yl)imidazo[1,2-a]pyridin-6-amine (67 mg, 219 μmol), pyrazolo[1,5-a]pyrimidine-3-carboxylic acid (53.6 mg, 329 μmol) in pyridine (550 μL) was added T3P® (698 mg, 1.10 mmol, 650 μL, 50% purity) at room temperature, stirred at rt for 24 h, diluted with EtOAc (10 mL) and water (10 mL). The layers were separated and the aqueous layer was extracted with EtOAc (2×10 mL), concentrated and the residue purification via acid modified, mass directed reverse phase HPLC obtain N-[8-fluoro-7-isopropoxy-2-(1-methyl-2-oxabicyclo[2.1.1]hexan-4-yl)imidazo[1,2-a]pyridin-6-yl]pyrazolo[1,5-a]pyrimidine-3-carboxamide (1.0 mg, 1.8 μmol, 0.81% yield, Trifluoroacetate salt). ¹H NMR (500 MHz, METHANOL-d₄) δ ppm 1.54 (s, 3H) 1.63 (d, J=6.10 Hz, 6H) 1.99 (dd, J=4.58, 1.53 Hz, 2H) 2.22 (dd, J=4.27, 1.83 Hz, 2H) 4.07 (s, 2H) 5.27 (td, J=5.95, 1.53 Hz, 1H) 7.35 (dd, J=6.71, 4.27 Hz, 1H) 8.03 (d, J=3.05 Hz, 1H) 8.74 (s, 1H) 8.93 (dd, J=4.27, 1.22 Hz, 1H) 9.21 (dd, J=7.02, 1.53 Hz, 1H) 9.70 (s, 1H) 10.75 (s, 1H).

### Assays

Compounds of the invention were assessed for their ability to inhibit IRAK4 activity. The inhibitory properties of the compounds of the invention described herein can be evidenced by testing in any one of the following assays.

### Biochemical Assay

The 2-hour 10 μM ATP Biochemical Assay employs a MesoScale Detection (MSD) format. The kinase reaction is based on the IRAK4 phosphorylation of a biotin labeled peptide (IRAK1 activation loop sequence 360-389).

The kinase reaction in 30 μl is carried out in wells of a 384 well polypropylene assay plate, with 1 nM IRAK4, 1.6 μM of biotinylated peptide substrate and 10 μM ATP in 50 mM Hepes, pH 7.5, 60 mM NaCl, 5 mM MgCl₂, 0.25 mM MnCl₂, 2 mM DTT, 0.01% BSA, 0.01% Tween-20, and 1% DMSO (from compound DMSO stocks), for 2 hours at room temperature. The activity is quenched with 11 μl of 70 mM EDTA, pH 8.

To detect the phosphorylated biotinylated peptide substrate, 30 μl of the quenched reaction mixture is added to equivalent wells of a 384 well streptavidin coated MesoScale plate (Meso Scale Discovery #L21SA-1). After a 1 hour incubation of the plate for 1 hour at room temperature with gentle mixing, the plate wells are washed 3 times with 50 mM Tris, pH 7.5, 150 mM NaCl, 0.02% Tween-20.

A 25 μl volume of 1:500 anti-P-Threonine Rabbit polyclonal Antibody plus 1:500 Goat-anti-Rabbit Sulfo Tag Antibody (Meso Scale Discovery R32AB-1) in 50 mM Tris, pH 7.5, 150 mM NaCl, 0.02% Tween-20 plus 2% BSA is then added to each well. After a 1-hour incubation of the plate for 1 hour at room temperature with gentle mixing, the plate wells are washed, 3 times with 50 mM Tris, pH 7.5, 150 mM NaCl, 0.02% Tween-20. A 40 μl volume of 2×MSD Read Buffer (Meso Scale Discovery R92TC-1) is added to each well, and the plate is read immediately in an MSD Plate Reader (Meso Scale Discovery).

The 2-hour 1 mM ATP IRAK4 Biochemical assay was performed as described above, but with 100 pM IRAK4 and 1 mM ATP.

Potency Data Table:

| | IRAK4 MSD | IRAK4 MSD | | IRAK4 MSD | IRAK4 MSD |
|---|---|---|---|---|---|
| | BIOCHEMICAL | BIOCHEMICAL | | BIOCHEMICAL | BIOCHEMICAL |
| Ex. | (10 μM ATP) | (1 mM ATP) | Ex. | (10 μM ATP) | (1 mM ATP) |
| No. | IC50 (μM) | IC50 (μM) | No. | IC50 (μM) | IC50 (μM) |
| 1 | 0.003 | 0.011 | 289 | | 0.006 |
| 2 | 0.003 | 0.016 | 290 | | 0.013 |
| 3 | 0.006 | 0.009 | 291 | 0.003 | |
| 4 | 0.004 | 0.015 | 292 | | 0.001 |
| 5 | 0.003 | 0.003 | 293 | 0.031 | |
| 6 | 0.190 | | 294 | | 0.002 |
| 7 | 0.280 | | 295 | | 0.004 |
| 8 | 0.140 | | 296 | | 0.089 |
| 9 | 0.005 | 0.01 | 297 | | 0.004 |
| 10 | 0.059 | | 298 | | 0.004 |
| 11 | 0.079 | | 299 | | 0.007 |
| 12 | 0.000 | 0.001 | 300 | | 0.005 |
| 13 | 0.002 | 0.003 | 301 | | 0.005 |
| 14 | 0.000 | 0.001 | 302 | | 0.008 |
| 15 | 0.001 | 0.001 | 303 | | 0.038 |
| 16 | 0.001 | 0.001 | 304 | 0.006 | 0.013 |
| 17 | 0.005 | 0.016 | 305 | 0.026 | |
| 18 | 0.004 | 0.014 | 306 | | 0.007 |
| 19 | 0.009 | 0.044 | 307 | | 0.015 |
| 20 | 0.003 | 0.006 | 308 | | 0.043 |
| 21 | 0.000 | 0 | 309 | | 0.001 |
| 22 | 0.001 | 0.002 | 310 | | 0 |
| 23 | 0.001 | 0.002 | 311 | | 0.006 |
| 24 | 0.002 | 0.004 | 312 | | 0.052 |
| 25 | 0.002 | 0.004 | 313 | | 0.067 |
| 26 | 0.003 | 0.007 | 314 | | 0.013 |
| 27 | 0.001 | 0.001 | 315 | | 0.021 |
| 28 | 0.043 | | 316 | | 0.003 |
| 29 | 0.023 | | 317 | | 0.001 |
| 30 | 0.048 | | 318 | | 0.005 |
| 31 | 0.089 | | 319 | | 0.001 |
| 32 | 0.064 | | 320 | | 0 |
| 33 | 0.003 | 0.011 | 321 | | 0.003 |
| 34 | 0.023 | | 322 | | 0 |
| 35 | 2.500 | | 323 | | 0.073 |
| 36 | 0.260 | | 324 | | 0.002 |
| 37 | 1.700 | | 325 | | 0.002 |
| 38 | 2.600 | | 326 | | 0.012 |
| 39 | 0.016 | | 327 | | 0.02 |
| 40 | 0.340 | | 328 | | 0.004 |
| 41 | 0.140 | | 329 | | 0.017 |
| 42 | 1.300 | | 330 | 0.023 | 0.038 |
| 43 | 2.600 | | 331 | | 0.031 |
| 44 | 0.012 | 0.027 | 332 | | 0.108 |
| 45 | 2.300 | | 333 | | 0.044 |
| 46 | 0.083 | | 334 | | 0.008 |
| 47 | 1.100 | | 335 | | 0.011 |
| 48 | 2.000 | | 336 | | 0.003 |
| 49 | 0.006 | 0.019 | 337 | | 0.006 |
| 50 | 0.004 | 0.014 | 338 | | 0.002 |
| 51 | 0.210 | | 339 | | 0.016 |
| 52 | 0.033 | | 340 | | 0.006 |
| 53 | 0.110 | | 341 | | 0.006 |
| 54 | 0.140 | | 342 | | 0.055 |
| 55 | 0.003 | 0.015 | 343 | | 0.004 |
| 56 | 0.005 | 0.014 | 344 | | 0.001 |
| 57 | 0.003 | 0.008 | 345 | | 0 |
| 58 | 0.110 | | 346 | | 0.001 |
| 59 | 0.002 | 0.003 | 347 | | 0 |
| 60 | 0.002 | 0.006 | 348 | | 0.001 |
| 61 | 0.001 | 0.002 | 349 | | 0.001 |
| 62 | 0.001 | 0.001 | 350 | | 0.006 |
| 63 | 0.000 | 0 | 351 | | 0.1 |
| 64 | 0.680 | | 352 | | 0.001 |
| 65 | 0.001 | 0.001 | 353 | | 0.011 |
| 66 | 0.000 | 0 | 354 | | 0.438 |
| 67 | 0.001 | 0 | 355 | 0.008 | 0.042 |
| 68 | 0.001 | 0.001 | 356 | 0.015 | 0.033 |
| 69 | 0.001 | 0.001 | 357 | | 0.03 |
| 70 | 0.150 | | 358 | 0.006 | 0.014 |
| 71 | 0.660 | | 359 | 0.01 | 0.023 |
| 72 | 0.001 | 0.001 | 360 | 0.006 | 0.005 |
| 73 | 0.011 | 0.032 | 361 | 0.002 | 0.01 |
| 74 | 0.007 | 0.018 | 362 | 0.006 | 0.017 |
| 75 | 0.001 | 0 | 363 | 0.029 | |
| 76 | 0.170 | | 364 | 0.004 | 0.013 |
| 77 | 0.000 | 0.001 | 365 | 0.002 | 0.006 |
| 78 | 0.000 | 0.001 | 366 | 0.003 | 0.004 |
| 79 | 0.001 | 0.001 | 367 | | 0.011 |
| 80 | 0.002 | 0.005 | 368 | 0.008 | 0.016 |
| 81 | 0.001 | 0.001 | 369 | 0.002 | 0.026 |
| 82 | 0.001 | 0.004 | 370 | 0.017 | |
| 83 | 0.001 | 0.005 | 371 | | 0.035 |
| 84 | 0.082 | 0.015 | 372 | 0.001 | 0.002 |
| 85 | 0.002 | 0.012 | 373 | 0.008 | 0.028 |
| 86 | 0.006 | 0.008 | 374 | | 0.002 |
| 87 | 0.004 | 0.017 | 375 | | 0.004 |
| 88 | 0.003 | 0.004 | 376 | | 0.009 |
| 89 | 0.000 | 0 | 377 | | 0.001 |
| 90 | 0.002 | 0.004 | 378 | | 0 |
| 91 | 0.001 | 0.003 | 379 | | 0.002 |
| 92 | 0.076 | | 380 | | 0.002 |
| 93 | 0.001 | 0.005 | 381 | | 0.004 |
| 94 | 0.001 | 0.004 | 382 | | 0.022 |
| 95 | 0.001 | 0.003 | 383 | | 0.014 |
| 96 | 0.001 | 0.001 | 384 | | 0.004 |
| 97 | 0.002 | 0.002 | 385 | | 0 |
| 98 | 0.008 | 0.018 | 386 | | 0.002 |
| 99 | 0.002 | 0.004 | 387 | | 0.001 |
| 100 | 0.008 | 0.011 | 388 | | 0 |
| 101 | 0.006 | 0.029 | 389 | | 0.016 |
| 102 | 0.061 | | 390 | | 0.072 |
| 103 | 0.023 | | 391 | | 0.021 |
| 104 | 0.130 | | 392 | | 0.008 |
| 105 | 0.006 | 0.015 | 393 | | 0.003 |
| 106 | 0.027 | | 394 | | 0.001 |
| 107 | 0.084 | | 395 | | 0.001 |
| 108 | 0.011 | 0.025 | 396 | | 0.002 |
| 109 | 0.002 | 0.003 | 397 | | 0.021 |
| 110 | 0.001 | 0.001 | 398 | | 0 |
| 111 | 0.002 | 0.003 | 399 | | 0.001 |
| 112 | 0.001 | 0.001 | 400 | | 0.054 |
| 113 | 0.001 | 0 | 401 | | 0.027 |
| 114 | 0.001 | 0 | 402 | | 0.157 |
| 115 | 0.001 | 0.002 | 403 | | 0 |
| 116 | 0.001 | 0.001 | 404 | | 0.004 |
| 117 | 0.027 | 0.063 | 405 | | 0.006 |
| 118 | 0.063 | | 406 | | 0.032 |
| 119 | 0.009 | 0.021 | 407 | | 0.732 |
| 120 | 0.028 | | 408 | | 0.025 |
| 121 | 2.205 | | 409 | | 0.534 |
| 122 | 0.008 | 0.003 | 410 | 0.011 | |
| 123 | 0.008 | 0.014 | 411 | 0.003 | |
| 124 | 0.002 | 0.002 | 412 | 0.037 | |
| 125 | 0.004 | 0.01 | 413 | 0.005 | 0.015 |
| 126 | 0.350 | | 414 | | 0.01 |
| 127 | 0.001 | 0.003 | 415 | | 0.033 |
| 128 | 0.002 | 0.007 | 416 | | 0.023 |
| 129 | 0.001 | 0 | 417 | | 0.022 |
| 130 | 0.001 | 0.001 | 418 | | 0.021 |
| 131 | 0.005 | 0.044 | 419 | | 0.011 |
| 132 | 0.018 | | 420 | | 0.071 |
| 133 | 0.002 | 0.004 | 421 | | 0.018 |
| 134 | 0.002 | 0.009 | 422 | | 0.012 |
| 135 | 0.002 | 0.006 | 423 | | 0.092 |
| 136 | 0.001 | 0.002 | 424 | | 0.017 |
| 137 | 0.001 | 0.001 | 425 | | 0.003 |
| 138 | 0.001 | 0.001 | 426 | | 0.002 |
| 139 | 0.001 | 0.001 | 427 | | 0.003 |
| 140 | 0.001 | 0.001 | 428 | | 0.01 |
| 141 | 0.001 | 0.001 | 429 | | 0.006 |
| 142 | 0.001 | 0.001 | 430 | | 0.008 |
| 143 | 0.001 | 0.002 | 431 | | 0.001 |
| 144 | 0.001 | 0.001 | 432 | | 0.005 |
| 145 | 0.001 | 0.002 | 433 | | 0.013 |
| 146 | 0.001 | 0.001 | 434 | | 0.004 |
| 147 | 0.011 | 0.051 | 435 | | 0.012 |
| 148 | 0.023 | | 436 | | 0.029 |
| 149 | 0.002 | 0.003 | 437 | | 0.004 |
| 150 | 0.001 | 0.002 | 438 | | 0.007 |
| 151 | 0.001 | 0.002 | 439 | | 0.063 |
| 152 | 0.004 | 0.004 | 440 | | 0.047 |
| 153 | 0.001 | 0.001 | 441 | | 0.118 |
| 154 | 0.220 | | 442 | | 0.024 |
| 155 | 0.005 | 0.016 | 443 | | 0.023 |
| 156 | 0.001 | 0.003 | 444 | | 0.039 |
| 157 | 2.300 | | 445 | | 0.012 |
| 158 | 2.900 | | 446 | | 0.008 |
| 159 | 0.006 | 0.051 | 447 | | 0.015 |
| 160 | 0.001 | 0.002 | 448 | | 0.023 |
| 161 | 0.400 | | 449 | | 0.064 |
| 162 | 0.370 | | 450 | 0.003 | |
| 163 | 0.002 | 0.007 | 451 | 0.029 | 0.066 |
| 164 | 0.003 | 0.005 | 452 | 0.031 | |
| 165 | 0.002 | 0.006 | 453 | 0.005 | |
| 166 | 0.002 | 0.009 | 454 | | 0.014 |
| 167 | 0.001 | 0 | 455 | | 0.043 |
| 168 | 0.007 | 0.015 | 456 | | 0.014 |
| 169 | 0.003 | 0.009 | 457 | | 0.096 |
| 170 | 0.004 | 0.012 | 458 | | 0.036 |
| 171 | 0.002 | 0.004 | 459 | | 0.022 |
| 172 | 0.002 | 0.004 | 460 | | 0.025 |
| 173 | 0.001 | 0.001 | 461 | 0.033 | 0.148 |
| 174 | | 0 | 462 | | 0.095 |
| 175 | 0.001 | 0 | 463 | | 0.104 |
| 176 | 0.001 | 0 | 464 | | 0.205 |
| 177 | | 0.001 | 465 | | 0.037 |
| 178 | 0.001 | 0.001 | 466 | | 0.059 |
| 179 | 0.001 | 0.001 | 467 | | 0.051 |
| 180 | 0.001 | 0.001 | 468 | | 0.022 |
| 181 | 0.001 | 0.002 | 469 | | 0.008 |
| 182 | 0.001 | 0.004 | 470 | | 0.006 |
| 183 | 0 | 0.001 | 471 | | 0.022 |
| 184 | 0.001 | 0.002 | 472 | | 0.015 |
| 185 | 0.001 | 0.002 | 473 | | 0.031 |
| 186 | 0.001 | 0.001 | 474 | | 0.025 |
| 187 | | 0.001 | 475 | | 0.008 |
| 188 | | 0.001 | 476 | | 0.006 |
| 189 | | 0 | 477 | | 0.015 |
| 190 | | 0.001 | 478 | | 0.012 |
| 191 | | 0.002 | 479 | | 0.015 |
| 192 | | 0.002 | 480 | | 0.005 |
| 193 | | 0.001 | 481 | | 0.025 |
| 194 | | 0.002 | 482 | | 0.015 |
| 195 | | 0.002 | 483 | | 0.022 |
| 196 | | 0.001 | 484 | | 0.02 |
| 197 | | 0.004 | 485 | | 0.037 |
| 198 | | 0.001 | 486 | | 0.016 |
| 199 | | 0.016 | 487 | | 0.001 |
| 200 | | 0.06 | 488 | | 0.002 |
| 201 | | 0.004 | 489 | | 0.003 |
| 202 | 0.002 | 0.004 | 490 | | 0.001 |
| 203 | 0.001 | 0.001 | 491 | | 0 |
| 204 | 0.004 | 0.021 | 492 | | 0 |
| 205 | 0.003 | 0.012 | 493 | | 0.001 |
| 206 | 0.005 | 0.019 | 494 | | 0.006 |
| 207 | 0.021 | | 495 | | 0.007 |
| 208 | 0.002 | 0.008 | 496 | | 0.001 |
| 209 | 0.005 | 0.009 | 497 | | 0.006 |
| 210 | 0.015 | 0.038 | 498 | | 0.001 |
| 211 | 0.007 | 0.022 | 499 | | 0.001 |
| 212 | 0.022 | | 500 | | 0.005 |
| 213 | 0.005 | 0.019 | 501 | | 0.002 |
| 214 | 0.003 | 0.007 | 502 | | 0.001 |
| 215 | 0.008 | 0.037 | 503 | | 0.002 |
| 216 | 0.007 | 0.023 | 504 | | 0.002 |
| 217 | 0 | 0.001 | 505 | | 0.007 |
| 218 | 0.001 | 0.002 | 506 | 0.004 | |
| 219 | 0.007 | 0.055 | 507 | 0.002 | 0.004 |
| 220 | 0.004 | 0.02 | 508 | 0.002 | |
| 221 | 0.001 | 0.011 | 509 | 0.002 | |
| 222 | 0.01 | 0.041 | 510 | 0.001 | 0.002 |
| 223 | 0.004 | 0.01 | 511 | 0.004 | |
| 224 | 0.001 | 0.003 | 512 | 0.004 | |
| 225 | 0.009 | 0.029 | 513 | 0.015 | |
| 226 | 0.035 | | 514 | 0.016 | |
| 227 | 0.001 | 0.001 | 515 | | 0.001 |
| 228 | 0.013 | 0.056 | 516 | 0.007 | 0.034 |
| 229 | 0.025 | | 517 | | 0 |
| 230 | 0.001 | 0.003 | 518 | | 0.001 |
| 231 | 0.001 | 0.007 | 519 | | 0.008 |
| 232 | | 0.044 | 520 | 0.001 | 0.005 |
| 233 | | 0 | 521 | 0.001 | 0.001 |
| 234 | 0.011 | 0.049 | 522 | 0.001 | 0.002 |
| 235 | | 0.023 | 523 | 0.001 | 0.001 |
| 236 | 0.006 | 0.019 | 524 | 0.004 | 0.015 |
| 237 | 0.001 | 0.005 | 525 | | 0.002 |
| 238 | 0.004 | 0.015 | 526 | | 0.001 |
| 239 | 0.002 | 0.003 | 527 | | 0.003 |
| 240 | 0.006 | 0.019 | 528 | | 0.004 |
| 241 | 0.01 | 0.041 | 529 | | 0.001 |
| 242 | 0.001 | 0.003 | 530 | | 0.045 |
| 243 | 0.003 | 0.014 | 531 | | 0 |
| 244 | | 0.028 | 532 | | 0 |
| 245 | 0.002 | | 533 | | 0 |
| 246 | 0.001 | 0.001 | 534 | | 0 |
| 247 | 0 | 0 | 535 | | 0.05 |
| 248 | | 0 | 536 | | 0 |
| 249 | 0.001 | 0.001 | 537 | | 0 |
| 250 | 0.001 | 0.001 | 538 | | 0.003 |
| 251 | 0.002 | | 539 | | 0.002 |
| 252 | 0.001 | | 540 | | 0 |
| 253 | 0.002 | 0.005 | 541 | | 0 |
| 254 | 0.002 | 0.005 | 542 | | 0 |
| 255 | 0 | 0 | 543 | | 0 |
| 256 | 0.001 | 0.001 | 544 | 0.011 | |
| 257 | 0 | 0.001 | 545 | | 0.007 |
| 258 | | 0.001 | 546 | | 0.001 |
| 259 | | 0 | 547 | | 0.001 |
| 260 | | 0 | 548 | | 0.001 |
| 261 | | 0.002 | 549 | | 0.005 |
| 262 | | 0.006 | 550 | | 0.055 |
| 263 | | 0.001 | 551 | | 0.026 |
| 264 | | 0.002 | 552 | | 0.017 |
| 265 | | 0 | 553 | | 0.011 |
| 266 | | 0 | 554 | | 0.011 |
| 267 | | 0.002 | 555 | | 0.007 |
| 268 | | 0.002 | 556 | | 0.005 |
| 269 | | 0.004 | 557 | | 0.006 |
| 270 | | 0.003 | 558 | | 0.044 |
| 271 | | 0.016 | 559 | | 0.012 |
| 272 | | 0.003 | 560 | | 0.002 |
| 273 | | 0.02 | 561 | | 0.051 |
| 274 | | 0.068 | 562 | | 0.053 |
| 275 | | 0.038 | 563 | | 0.046 |
| 276 | | 0.005 | 564 | | 0.032 |
| 277 | | 0.007 | 565 | | 0.01 |
| 278 | | 0.023 | 566 | | 0.054 |
| 279 | | 0 | 567 | | 0.008 |
| 280 | | | 568 | | 0.009 |
| 281 | | | 569 | | 0.004 |
| 282 | | 0.007 | 570 | | 0.005 |
| 283 | | | 571 | | 0 |
| 284 | | | 572 | | 0 |
| 285 | | 0.006 | 573 | | 0.003 |
| 286 | | 0.005 | 574 | | 0.002 |
| 287 | | 0.006 | 575 | | 0.012 |
| 288 | | 0.008 | 576 | | 0.199 |
| | | | 577 | 0.003 | 0.001 |

What is claimed is:

1. A compound of formula (I′): 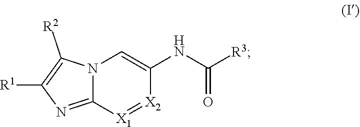
or a pharmaceutically acceptable salt thereof, wherein:
R¹ is selected from the group consisting of halogen, C₁₋₅ alkyl, C₃₋₆ cycloalkyl, —C₁₋₂ alkyl-C₃₋₆ cycloalkyl, C(O)NR⁴R⁵, C₁₋₄ alkyl-NR⁴R⁵, a fully saturated 4 to 7 membered heterocycle containing 1 to 2 heteroatoms independently selected from nitrogen, sulfur and oxygen, 5 to 6 membered heteroaryl, phenyl, —C₁₋₂ alkyl-C₄₋₇ heterocycle, wherein the C₄₋₇ heterocycle may be fully or partially saturated and contains 1 to 2 heteroatoms independently selected from nitrogen, sulfur and oxygen, —C₁₋₂ alkyl-C₅₋₆heteroaryl wherein the heteroaryl contains 1, 2 or 3 heteroatoms independently selected from nitrogen, sulfur and oxygen, —C₁₋₄ alkyl-O—C₁₋₂ alkyl, —C₁₋₂ alkyl-O—C₅₋₆ heteroaryl wherein the heteroaryl contains 1 or 2 heteroatoms independently selected from nitrogen, sulfur and oxygen, a fully saturated 5 to 10 membered bridged-carbocyclic ring, a 5 to 10 membered bridged-heterocyclic ring system having 1 to 2 heteroatoms independently selected from nitrogen and oxygen, a 5 to 10 membered fused heterobicyclic ring system having 1 to 2 heteroatoms independently selected from nitrogen and oxygen, and a 5 to 10 membered spiro heterobicyclic ring system having 1 to 2 heteroatoms independently selected from nitrogen and oxygen, wherein R¹ may be optionally substituted with 1, 2 or 3 substituents which are independently selected from halo, nitrile, oxo, —C(O)OH, halo-substitutedC₁₋₄ alkyl, hydroxy-substitutedC₁₋₄ alkyl, C₁₋₄ alkyl, C₄₋₇heterocycle containing 1 to 2 heteroatoms independently selected from nitrogen and oxygen, C₁₋₄ alkyl-O—C₁₋₂ alkyl, hydroxy, C(O)R⁶ and C₁₋₄ alkoxy;
R² is hydrogen, C₁₋₄ alkyl or halogen; or
R¹ and R² are taken together with their intervening atoms to form a 4 to 7 membered partially saturated carbocyclic ring or a partially saturated heterocyclic ring having one nitrogen said nitrogen may be optionally substituted with C₁₋₄ alkyl;
R⁴ and R⁵ are independently selected from hydrogen, hydroxy-substituted-C₁₋₄ alkyl and C₁₋₄ alkyl; or
R⁴ and R⁵ are taken together with the nitrogen to which they are connected form a 4 to 7 membered saturated heterocyclic ring, said ring optionally having an additional heteroatom selected from nitrogen and oxygen wherein said nitrogen may be optionally substituted with C₁₋₄ alkyl;
R⁶ is C₁₋₄ alkyl, halo-substituted-C₁₋₄ alkyl or O—C₁₋₄ alkyl;
R³ is selected from the group consisting of
ix. a 5 or 6 membered heteroaryl having 1 to 2 heteroatoms independently selected from nitrogen, oxygen and sulfur, said heteroaryl is optionally substituted with 1 to 3 substituents independently selected from the group consisting of R⁷;
x. Phenyl optionally substituted with 1 to 3 substituents independently selected from the group consisting of R⁷;
xi. a 6 membered partially saturated heterocycle having 1 to 2 nitrogen atoms, said heterocycle may be optionally substituted with 1 to 3 substituents independently selected from the group consisting of R⁷; and
xii. an 8 to 10 membered fused bicyclic ring system optionally having 1, 2 or 3 heteroatoms independently selected from nitrogen and oxygen, said ring system is optionally substituted with 1 to 3 substituents independently selected from the group consisting of R⁷;
X₁ and X₂ are independently selected from N, CH and CR⁸; provided at least one of X₁ and X2 is CR⁸;
R⁸ is selected from halogen, nitrile, NR⁴R⁵, —OR⁹ and a partially or fully saturated 4 to 7 membered heterocycle which contains 1 or 2 heteroatoms independently selected from nitrogen and oxygen, said C₁₋₄alkyl and said heterocycle is optionally substituted with 1 to 3 substituents selected from R^{4a};
R^{4a} for each occurrence, is independently selected from hydrogen, oxo, C₁₋₄ alkyl, halo-substitutedC₁₋₄ alkyl, C₁₋₄alkoxy, and C₃₋₆cycloalkyl; or two R^{4a} groups taken together with the carbon to which they are attached may combine to form a spiro 3-8 membered cycloalkyl;
R⁹ is hydrogen, a C₃₋₆cycloalkyl, a partially or fully saturated 4 to 7 membered heterocycle which contains 1 or 2 heteroatoms independently selected from nitrogen and oxygen, or a C₁₋₅alkyl, wherein said C₃₋₆cycloalkyl, said partially or fully saturated 4 to 7 membered heterocycle and said C₁₋₅alkyl represented by R⁹ are optionally substituted with 1 to 3 substituents independently selected from halogen, hydroxyl, nitrile, oxo, NR⁴R⁵, halo-substitutedC₁₋₄alkyl, C₁₋₄alkoxy, halo-substituted C₁₋₄alkoxy, C₃₋₆cycloalkyl, phenyl and a 4 to 7 membered partially or fully saturated heterocycle containing 1 or 2 heteroatoms selected from nitrogen and oxygen, wherein said C₃₋₆cycloalkyl, phenyl and 4 to 7 membered heterocycle may be optionally substituted with 1 to 3 R¹⁰;
R¹⁰ is independently selected from oxo, halo, halo-substitutedC₁₋₄ alkyl and C₁₋₄ alkyl;
R⁷ for each occurrence, is independently selected from C₁₋₄ alkyl, nitrile, oxo, halo, halo-substitutedC₁₋₄alkyl, —NR¹¹R¹², C₁₋₄ alkoxy, halo-substitutedC₁₋₄ alkoxy, a C₃₋₆cycloalkyl, a C₃₋₆cycloalkoxy, 4 to 7 membered partially or fully saturated heterocycle containing 1 or 2 heteroatoms selected from nitrogen and oxygen, and a 5 or 6 membered heteroaryl having 1 to 2 heteroatoms independently selected from nitrogen, oxygen and sulfur, said C₃₋₆cycloalkyl and heteroaryl may be optionally substituted with 1 to 2 substituents independently selected from the group consisting of C₁₋₄ alkyl, hydroxy and halogen and said C₁₋₄ alkyl and C₁₋₄alkoxy may be optionally substituted with C₁₋₄alkoxy; and
R¹¹ and R¹² are each independently selected from hydrogen, —C(O)C₁₋₄ alkyl and C₁₋₄ alkyl; or R¹¹ and R¹² may combine to form a 4 to 6 membered saturated ring optionally containing one additional heteroatom selected from nitrogen or oxygen wherein said additional nitrogen may be optionally substituted with C₁₋₄ alkyl,
provided when X₁ is CR⁸, X₂ is CH, and R⁸ is halogen, then R⁷ is not a 4 to 7 membered partially or fully saturated heterocycle containing 1 or 2 heteroatoms selected from nitrogen and oxygen; and provided that the compound is not:

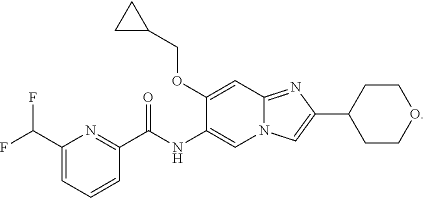 [SMILES: O=C(Nc1cn2cc(C3CCOCC3)nc2cc1OCC1CC1)c1cccc(C(F)F)n1]

2. A compound of formula (I): 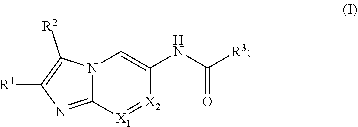
or a pharmaceutically acceptable salt thereof, wherein:
R¹ is selected from the group consisting of C₁₋₅ alkyl, C₃₋₆cycloalkyl, —C₁₋₂ alkyl-C₃₋₆cycloalkyl, C(O)NR⁴R⁵, C₁₋₄ alkyl-NR⁴R⁵, a fully saturated 4 to 7 membered heterocycle containing 1 to 2 heteroatoms independently selected from nitrogen, sulfur and oxygen, —C₁₋₂ alkyl-C₄₋₇ heterocycle, wherein the C₄₋₇heterocycle may be fully or partially saturated and contains 1 to 2 heteroatoms independently selected from nitrogen, sulfur and oxygen, —C₁₋₂ alkyl-C₅₋₆heteroaryl wherein the heteroaryl contains 1, 2 or 3 heteroatoms independently selected from nitrogen, sulfur and oxygen, —C₁₋₄ alkyl-O—C₁₋₂ alkyl, —C₁₋₂ alkyl-O—C₅₋₆heteroaryl wherein the heteroaryl contains 1 or 2 heteroatoms independently selected from nitrogen, sulfur and oxygen, a fully saturated 5 to 10 membered bridged-carbocyclic ring, a 5 to 10 membered fused heterobicyclic ring system having 1 to 2 heteroatoms independently selected from nitrogen and oxygen and a 5 to 10 membered spiro heterobicyclic ring system having 1 to 2 heteroatoms independently selected from nitrogen and oxygen, wherein R¹ may be optionally substituted with 1, 2 or 3 substituents which are independently selected from halo, nitrile, oxo, halo-substitutedC₁₋₄ alkyl, hydroxy-substitutedC₁₋₄ alkyl, C₁₋₄ alkyl, C₄₋₇heterocycle containing 1 to 2 heteroatoms independently selected from nitrogen and oxygen, C₁₋₄ alkyl-O—C₁₋₂ alkyl, hydroxy, C(O)R⁶ and C₁₋₄ alkoxy;
R² is hydrogen, C₁₋₄ alkyl or halogen; or
R¹ and R² are taken together with their intervening atoms to form a 4 to 7 membered partially saturated carbocyclic ring or a partially saturated heterocyclic ring having one nitrogen said nitrogen may be optionally substituted with C₁₋₄ alkyl;
R⁴ and R⁵ are independently selected from hydrogen, hydroxy-substituted-C₁₋₄ alkyl and C₁₋₄ alkyl; or
R⁴ and R⁵ are taken together with the nitrogen to which they are connected form a 4 to 7 membered saturated heterocyclic ring, said ring optionally having an additional heteroatom selected from nitrogen and oxygen wherein said nitrogen may be optionally substituted with C₁₋₄ alkyl;
R⁶ is C₁₋₄ alkyl, halo-substitutedC₁₋₄ alkyl or O—C₁₋₄ alkyl;
R³ is selected from the group consisting of
xiii. a 5 or 6 membered heteroaryl having 1 to 2 heteroatoms independently selected from nitrogen, oxygen and sulfur, said heteroaryl is optionally substituted with 1 to 3 substituents independently selected from the group consisting of R⁷;
xiv. Phenyl optionally substituted with 1 to 3 substituents independently selected from the group consisting of R⁷;
xv. a 6 membered partially saturated heterocycle having 1 to 2 nitrogen atoms, said heterocycle may be optionally substituted with 1 to 3 substituents independently selected from the group consisting of R⁷; and
xvi. an 8 to 10 membered fused bicyclic ring system optionally having 1, 2 or 3 heteroatoms independently selected from nitrogen and oxygen, said ring system is optionally substituted with 1 to 3 substituents independently selected from the group consisting of R⁷;
X₁ and X₂ are independently selected from N, CH and CR⁸, wherein only one of X₁ or X₂ may be CR⁸;
R⁸ is selected from halogen, nitrile, NR⁴R⁵, —OR⁹ and a partially or fully or partially saturated 4 to 7 membered heterocycle which contains 1 or 2 heteroatoms independently selected from nitrogen and oxygen, said heterocycle is optionally substituted with 1 to 3 substituents selected from R^{4a};
R^{4a} for each occurrence, is independently selected from hydrogen, oxo, C₁₋₄ alkyl, halo-substitutedC₁₋₄ alkyl and C₃₋₆cycloalkyl; or two R^{4a} groups taken together with the carbon to which they are attached may combine to form a spiro 3-8 membered cycloalkyl;
R⁹ is hydrogen or an optionally substituted C₁₋₅alkyl having 1 to 3 substituents independently selected from halogen, hydroxyl, NR⁴R⁵, C₁₋₄alkoxy, C₃₋₆cycloalkyl, phenyl and a 4 to 7 membered partially or fully saturated heterocycle containing 1 or 2 heteroatoms selected from nitrogen and oxygen, wherein said C₃₋₆cycloalkyl, phenyl and 4 to 7 membered heterocycle may be optionally substituted with 1 to 3 R¹⁰;
R¹⁰ is independently selected from oxo, halo, halo-substitutedC₁₋₄ alkyl and C₁₋₄ alkyl;
R⁷ for each occurrence, is independently selected from C₁₋₄ alkyl, oxo, halo, halo-substitutedC₁₋₄alkyl, —NR¹¹R¹², C₁₋₄ alkoxy, halo-substitutedC₁₋₄ alkoxy, a C₃₋₆cycloalkyl and a 5 or 6 membered heteroaryl having 1 to 2 heteroatoms independently selected from nitrogen, oxygen and sulfur, said C₃₋₆cycloalkyl and heteroaryl may be optionally substituted with 1 to 2 substituents independently selected from the group consisting of C₁₋₄ alkyl, hydroxy and halogen; and
R¹¹ and R¹² are each independently selected from hydrogen, —C(O)C₁₋₄ alkyl and C₁₋₄ alkyl; or R¹¹ and R¹² may combine to form a 4 to 6 membered saturated ring optionally containing one additional heteroatom selected from nitrogen or oxygen wherein said additional nitrogen may be optionally substituted with C₁₋₄ alkyl

3. The compound of claim 1 or 2 of formula (Ia): 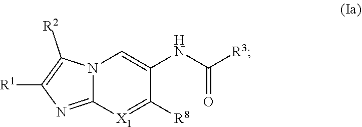
or a pharmaceutically acceptable salt thereof, where X₁ is N or CH.

4. The compound of claim 1 or 2 of formula (Ib): 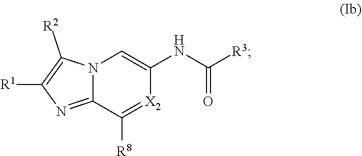
or a pharmaceutically acceptable salt thereof, wherein X₂ is N or CH.

5. The compound of any one of claims 1-4, or a pharmaceutically acceptable salt thereof, wherein:
R³ is selected from the group consisting of
i. a 5 to 6 membered heteroaryl selected from pyridyl, oxazolyl, pyrazinyl, oxadazoyl, thiazolyl, pyrazolyl and imidazolyl, said heteroaryl is optionally substituted with 1 to 2 substituents independently selected from the group consisting of R⁷;
ii. Phenyl optionally substituted with 1 to 2 substituents independently selected from the group consisting of R⁷;
iii. pyridinyl-2(1H)-one optionally substituted with 1 to 2 substituents independently selected from the group consisting of R⁷; and
iv. an 8 to 10 membered fused bicyclic ring system selected from the group consisting of 1,3-dihydroisobenzofuran, 4-methyl-3,4-dihydro-2H-benzo[b][1,4]oxazine, 5,6-dihydro-4H-pyrrolo[1,2-b]pyrazole, 6,7-dihydro-5H-cyclopenta[b]pyridine, pyrazolo[1,5-a]pyrimidine, indolin-2-one, 2,3-dihydrobenzofuran, 1-methyl-2-oxo-1,2,3,4-tetrahydroquinoline, 3,4-dihydroquinolin-2(1H)-one, and isochromane, wherein said fused bicyclic ring system is optionally substituted with 1 to 2 substituents independently selected from the group consisting of R⁷.

6. The compound of claim 1 or 2 of formula (IIa): 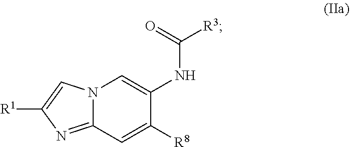
or a pharmaceutically acceptable salt thereof.

7. The compound of claim 1 or 2 of formula (IIc): 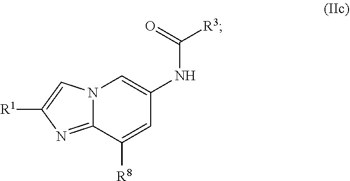
or a pharmaceutically acceptable salt thereof.

8. The compound of claim 1, or a pharmaceutically acceptable salt thereof, wherein the compound is represented by formula (IIe): 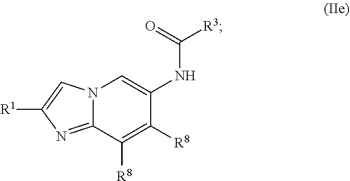
or a pharmaceutically acceptable salt thereof.

9. The compound of claim 1, or a pharmaceutically acceptable salt thereof, wherein the compound is represented by the following formula: 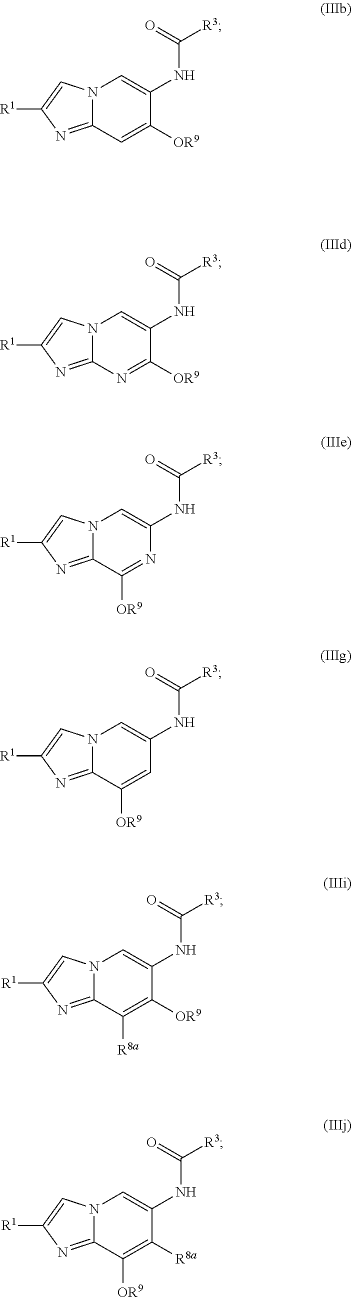
or a pharmaceutically acceptable salt thereof, wherein R^{8a} is C₁₋₄alkyl, halo-substituted C₁₋₄alkyl, or halogen.

10. The compound of claim 9, or a pharmaceutically acceptable salt thereof, wherein R⁹ is hydrogen or an optionally substituted C₁₋₅alkyl having 1 to 3 substituents independently selected from halogen, hydroxyl, NR⁴R⁵, C₁₋₄alkoxy, phenyl and a 4 to 7 membered partially or fully saturated heterocycle containing 1 or 2 heteroatoms selected from nitrogen and oxygen, wherein said phenyl and 4 to 7 membered heterocycle may be optionally substituted with 1 to 3 R¹⁰; and
R¹⁰ is independently selected from oxo, halo, halo-substitutedC₁₋₄ alkyl and C₁₋₄ alkyl.

11. The compound of claim 9, or a pharmaceutically acceptable salt thereof, wherein R⁹ is C₁₋₅alkyl.

12. The compound of any one of the preceding claims, or a pharmaceutically acceptable salt thereof, wherein:
R³ is selected from pyridyl, oxazolyl, pyrazinyl, oxadazoyl, thiazolyl, pyrazolyl, imidazolyl, said R³ is optionally substituted with 1 to 2 substituents independently selected from the group consisting of halo, halo-substitutedC₁₋₄ alkyl, —NR¹¹R¹², and C₁₋₄ alkyl;
or a pharmaceutically acceptable salt thereof.

13. The compound of any one of claim 1 to 11, or a pharmaceutically acceptable salt thereof, wherein:
R³ is pyridinyl-2(1H)-one optionally substituted with 1 to 2 substituents independently selected from the group consisting of halo, halo-substitutedC₁₋₄ alkyl, —NR¹¹R¹², and C₁₋₄ alkyl;
or a pharmaceutically acceptable salt thereof.

14. The compound of any one of claim 1 to 11, or a pharmaceutically acceptable salt thereof, wherein:
R³ is phenyl, said phenyl is optionally substituted with 1 to 2 substituents independently selected from the group consisting of halo, halo-substitutedC₁₋₄ alkyl, —NR¹¹R¹², and C₁₋₄ alkyl;
or a pharmaceutically acceptable salt thereof.

15. The compound of any one of claim 1 to 11, or a pharmaceutically acceptable salt thereof, wherein:
R³ is selected from the group consisting of 1,3-dihydroisobenzofuran, 4-methyl-3,4-dihydro-2H-benzo[b][1,4]oxazine, 5,6-dihydro-4H-pyrrolo[1,2-b]pyrazole, 6,7-dihydro-5H-cyclopenta[b]pyridine, indolin-2-one, 2,3-dihydrobenzofuran, pyrazolo[1,5-a]pyrimidine, 1-methyl-2-oxo-1,2,3,4-tetrahydroquinoline, 3,4-dihydroquinolin-2(1H)-one, and isochromane, wherein said R³ is optionally substituted with 1 to 2 substituents independently selected from the group consisting halo, halo-substitutedC₁₋₄ alkyl, —NR¹¹R¹², and C₁₋₄ alkyl;
or a pharmaceutically acceptable salt thereof.

16. The compound of any one of claim 1 to 11, or a pharmaceutically acceptable salt thereof, wherein R³ is an 8 to 10 membered fused bicyclic heteroaryl containing 1 to 3 heteroatoms independently selected from nitrogen and oxygen or an 8 to 10 membered fused heterobicyclic ring containing 1 to 3 heteroatoms independently selected from nitrogen and oxygen, wherein the 8 to 10 membered fused bicyclic heteroaryl and the 8 to 10 membered fused heterobicyclic ring represented by R³ is optionally substituted with 1 or 2 substituents independently selected from the group consisting halo, halo-substitutedC₁₋₄ alkyl, —NR¹¹R¹², C₁₋₄alkoxy and C₁₋₄ alkyl.

17. The compound of claim 16, or a pharmaceutically acceptable salt thereof, wherein R³ is selected from 2,3-dihydrofuro[2,3-b]pyridine, 5,6,7,8-tetrahydroindolizine, 2,3-dihydrothieno[3,4-b][1,4]dioxine, 6,7-dihydro-5H-cyclopenta[b]pyridine, 2,3-dihydrobenzofuran, 2,3-dihydro-1H-pyrrolizine, pyrazolo[1,5-a]pyridine, pyrazolo[1,5-a]pyrimidine, and [1,2,4]triazolo[1,5-a]pyridine, each of which is optionally substituted with 1 or 2 substituents independently selected from the group consisting of halo, halo-substitutedC₁₋₄ alkyl, —NR¹¹R¹², C₁₋₄alkoxy and C₁₋₄ alkyl.

18. The compound of any one of claims 1 to 11, or a pharmaceutically acceptable salt thereof, wherein:
R³ is pyridyl optionally substituted with 1 or 2 substituents independently selected from R⁷, R⁷ is nitrile, halo, C₁₋₄alkyl, halo-substitutedC₁₋₄alkyl, C₁₋₄alkoxy, halo-substitutedC₁₋₄alkoxy, C₃₋₆cycloalkyl, C₃₋₆cycloalkoxy, —NR¹¹R¹², and 4 to 7 membered fully saturated heterocycle containing 1 or 2 heteroatoms selected fro nitrogen and oxygen, wherein the C₁₋₄alkyl and C₁₋₄alkoxy is optionally substituted with C₁₋₄alkoxy;
R¹¹ and R¹² are each independently hydrogen or C₁₋₄alkyl.

19. The compound of claim 18, or a pharmaceutically acceptable salt thereof, wherein R³ is 2-pyridyl or 3-pyridyl, each of which is optionally substituted with 1 or 2 substituents independently selected from R⁷.

20. The compound of claim 18 or 19, or a pharmaceutically acceptable salt thereof, wherein R⁷, for each occurrence, is independently tetrahydrofuran, F, —CN, —CH₃, —CH(CH₃)₂, —CH₂CH₃, —CHF—CH₂F, —CHF₂, —CF₂CH₃, —CF₃, —OCH₃, —OCH(CH₃)₂, —OCHF₂, —OCH₂CF₃, —OCH₂CHF₂, —OCH₂CH₂OCH₃, —CH₂OCH₃, —O-cyclopropyl, —O-cyclobutyl, cyclopropyl, or —N(CH₃)₂.

21. The compound of any of one claim 1 or 2 of formula (IV): 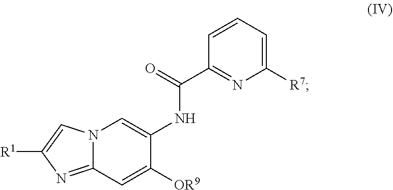
or a pharmaceutically acceptable salt thereof, wherein:
R⁹ is an optionally substituted C₁₋₅alkyl having 1 to 3 substituents independently selected from halogen, hydroxyl, NR⁴R⁵, C₁₋₄alkoxy, C₃₋₆cycloalkyl, phenyl and a 4 to 7 membered partially or fully saturated heterocycle containing 1 or 2 heteroatoms selected from nitrogen and oxygen, wherein said C₃₋₆cycloalkyl, phenyl and 4 to 7 membered heterocycle may be optionally substituted with 1 to 3 R¹⁰.

22. The compound of claim 21, or a pharmaceutically acceptable salt thereof, wherein:
R¹ is C₃₋₆cycloalkyl optionally substituted with 1 or 2 substituents independently selected from the group consisting of C₁₋₄alkyl, halogen, halo-substitutedC₁₋₄ alkyl, hydroxyl and C₁₋₄alkoxy, or R¹ is a C₁₋₅ alkyl which is optionally substituted with 1 or 3 substituents independently selected from the group consisting of halogen, halo-substitutedC₁₋₄ alkyl, hydroxy-substitutedC₁₋₄ alkyl, hydroxyl, C₁₋₄alkoxy and C₃₋₆cycloalkyl, wherein said C₃₋₆cycloalkyl is optionally substituted with 1 or 2 substituents independently selected from the group consisting of halogen, halo-substitutedC₁₋₄ alkyl, hydroxyl and C₁₋₄alkoxy.

23. The compound of claim 21, or a pharmaceutically acceptable salt thereof, wherein:
R¹ is an C₃₋₆cycloalkyl optionally substituted with 1 or 2 substituents independently selected from the group consisting of C₁₋₄alkyl, halogen, halo-substitutedC₁₋₄ alkyl, hydroxyl and C₁₋₄alkoxy.

24. The compound of claim 21, or a pharmaceutically acceptable salt thereof, wherein:
R¹ is a C₁₋₅ alkyl which is optionally substituted with 1 or 3 substituents independently selected from the group consisting of halogen, halo-substitutedC₁₋₄ alkyl, hydroxyl, C₁₋₄alkoxy and C₃₋₆cycloalkyl, wherein said C₃₋₆cycloalkyl is optionally substituted with 1 or 2 substituents independently selected from the group consisting of halogen, halo-substitutedC₁₋₄ alkyl, hydroxyl and C₁₋₄alkoxy.

25. The compound of claim 21, or a pharmaceutically acceptable salt thereof, wherein:
R¹ is an C₃₋₆cycloalkyl optionally substituted with 1 or 2 substituents independently selected from the group consisting of C₁₋₄alkyl, halogen, halo-substitutedC₁₋₄ alkyl, hydroxyl and C₁₋₄alkoxy; and
R³ is optionally substituted with 1 or 2 substituents independently selected from and C₁₋₄ alkyl and halo-substitutedC₁₋₄ alkyl.

26. The compound of any one of claims 1 to 20, or a pharmaceutically acceptable salt thereof, wherein R¹ is a 5 to 10 membered bridged-heterocyclic ring containing 1 to 3 heteroatoms independently selected from nitrogen and oxygen; wherein the 5 to 10 membered bridged-heterocyclic ring is optionally substituted with 1 or 3 substituents independently selected from C₁₋₄alkyl, halogen, halo-substitutedC₁₋₄ alkyl, hydroxyl and C₁₋₄alkoxy.

27. The compound of claim 26, or a pharmaceutically acceptable salt thereof, wherein R¹ is a 6 to 8 membered bridged-heterocyclic ring containing 1 or 2 heteroatoms independently selected from nitrogen and oxygen, wherein the 6 to 8 membered bridged-heterocyclic ring is optionally substituted with 1 or 2 substituents independently selected from C₁₋₄alkyl, halogen, halo-substitutedC₁₋₄ alkyl, hydroxyl and C₁₋₄alkoxy.

28. A compound of claim 26, or a pharmaceutically acceptable salt thereof, wherein R¹ is selected from 2-oxabicyclo[2.1.1]hexan-4-yl 8-oxabicyclo[3.2.1]octan-3-yl, 2-oxabicyclo[2.2.1]heptan-4-yl, 2-oxabicyclo[2.2.2]octan-4-yl, 2-oxabicyclo[3.1.1]heptan-5-yl, each of which is optionally substituted with 1 to 3 substituents independently selected from C₁₋₄alkyl, halogen, halo-substitutedC₁₋₄ alkyl, hydroxyl and C₁₋₄alkoxy.

29. The compound of claim 27 or 28, or a pharmaceutically acceptable salt thereof, wherein R¹ is optionally substituted with 1 to 3 substituents independently selected from —CH₃ and —CH₂F.

30. The compound of any one of claims 1 to 20, or a pharmaceutically acceptable salt thereof, wherein:
R¹ is selected from halogen, —C(═O)NR⁴R⁵, C₁₋₄alkyl, C₃₋₆cycloalkyl, —C₁₋₂alkyl-C₃₋₆cycloalkyl, —C₁₋₄alkyl-O—C₁₋₂alkyl, —C₁₋₂alkyl-C₄₋₇heterocycle, —C₁₋₂alkyl-C₅₋₆heteroaryl, —C₁₋₂alkyl-O—C₅₋₆heteroaryl, a fully saturated 4 to 6 membered heterocycle, 5 to 6 membered heteroaryl, phenyl, 5 to 6 membered bridged-carbocyclic ring, 6 to 8 membered fused heterobicyclic ring, 6 to 8 membered spiro heterobicyclic ring, and 6 to 8 membered bridged heterobicyclic ring, wherein R¹ is optionally substituted with 1 to 3 substituents independently selected from halo, hydroxyl, nitrile, C₁₋₄alkyl, C₁₋₄alkoxy, halo-substitutedC₁₋₄alkyl, hydroxyl-substitutedC₁₋₄alkyl, C₁₋₄alkyl-O—C₁₋₂alkyl, —C(═O)OH, —C(═O)—C₁₋₄alkyl, —C(═O)OC₁₋₄alkyl, and 4 to 6 membered heterocycle;
R⁴ and R⁵ are each independently H or C₁₋₄alkyl.

31. The compound of claim 30, wherein R¹ is selected from —Cl, —C(═O)N(CH₃)₂, —CH₃, —CH₂CH₃, —CH₂F, —CHF₂, —CF₂CH₃, —CH₂CH₂CH₂OCH₃, —CF₂CH₂CH₂OCH₃, —CH₂OCH₂CH₃, —CH₂OCH₃, —C(CH₃)₂CH₂OCH₃, —C(CH₃)₂OH, —CH₂CH₂CH₂OH, —CH₂CH₂C(CH₃)₂OH, —CH₂CHFCH₂OH, —CH₂CH₂OCF₃, —CH₂O-(pyridin-2-yl), —CH₂-(1,2,4-triazol-1-yl), —C(CH₃)₂CN, —CH(CH₃)CN, —C(CH₃)₂C(═O)OCH₃, —C(CH₃)₂C(═O)OH, —CH(OH)-cyclopropyl, —CH₂-tetrahydrofuran, —CH(CH₃)-tetrahydrofuran, —CHF-tetrahydrofuran, —CH₂-tetrahydropyranyl, —CH₂-(1,4-dixoan-2-yl), —CH₂-(2-oxopyrrolidin-1-yl), —CH₂-(pyridin-2-yloxy), —CH₂-morpholinyl, C₃₋₆cycloalkyl selected from cyclopropyl, cyclobutyl, cyclopentyl, and cyclohexyl, bicyclo[1.1.1]pentan-1-yl optionally substituted with F, —CH₃, —OCH₃, —CN, CHF₂, —C(CH₃)₂OH, —CH₂OH, and —CH₂OCH₃, 6 to 8 membered fused heterobicyclic ring selected from 3-oxabicyclo[4.1.0]heptan-6-yl, 3-oxabicyclo[3.1.0]hexan-6-yl, and 3-oxabicyclo[3.1.0]hexan-1-yl, 6 to 8 membered fused heterobicyclic ring selected from 5-oxaspiro[2.4]heptan-1-yl, 6-oxaspiro[2.5]octan-1-yl, and 6-oxaspiro[3.4]octan-2-yl, a fully saturated 4 to 6 membered heterocycle selected from tetrahydrofuran-3-yl, tetrahydro-2H-pyran-4-yl, tetrahydro-2H-pyran-3-yl, azetidinyl, tetrahydrothiophen-3-yl, pyrrolidin-3-yl, 2-oxopyrrolidin-3-yl, piperidin-4-yl, 1-methyl-6-oxopiperidin-2-yl, morpholinyl, and 1,4-dioxan-2-yl, phenyl optionally substituted with —F, pyrazolyl optionally substituted with 1 or 2 substituents independently selected from —CH₃ and —CHF₂, a 6 to 8 membered bridged heterobicyclic ring selected from 2-oxabicyclo[2.1.1]hexan-4-yl 8-oxabicyclo[3.2.1]octan-3-yl, 2-oxabicyclo[2.2.1]heptan-4-yl, 2-oxabicyclo[2.2.2]octan-4-yl, wherein the C₃₋₆cycloalkyl is optionally substituted with 1 to 2 substituents independently selected from F, —CN, —OH, —OCH₃, —OCH(CH₃)₂, and —CH₂OH, optionally substituted with 1 to 2 substituents independently selected from —CH₃, OCH₂CH₃, —OH, CN, —CH₂CHF₂, —CH₂CH₂OCH₃, —C(═O)CH₃, and oxetan-3-yl, and the 6 to 8 membered bridged heterobicyclic ring is optionally substituted with 1 to 2 substituents independently selected form —CH₃.

32. The compound of claim 1, or a pharmaceutically acceptable salt thereof, wherein the compound is represented by formula (V), (VI), (VII) or (VIII): 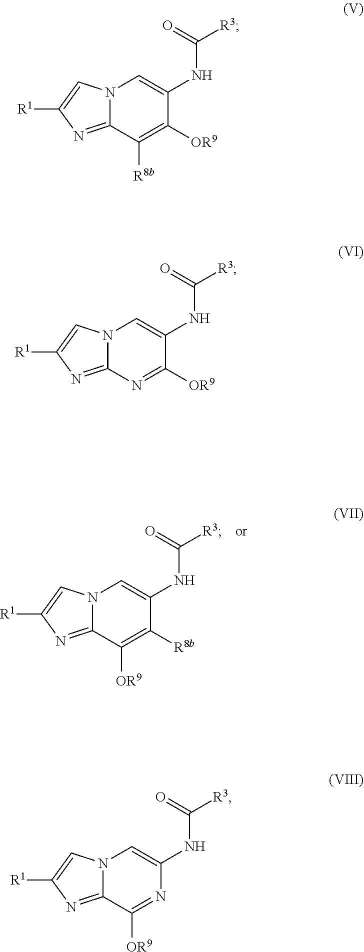
or a pharmaceutically acceptable salt thereof, wherein:
R¹ is a 6 to 8 membered bridged-heterocyclic ring containing 1 to 2 heteroatoms independently selected from oxygen and nitrogen, wherein the 6 to 8 membered bridged-heterocyclic ring is optionally substituted with 1 or 2 substituents independently selected from C₁₋₄alkyl, halogen, halo-substitutedC₁₋₄ alkyl, hydroxyl and C₁₋₄alkoxy;
R³ is a pyridyl, pyridinyl-2(1H)-one, a 9 to 10 membered fused heterobicyclic ring having 1 to 2 heteroatoms independently selected from oxygen and nitrogen, or a 9 to 10 membered fused bicyclic heteroaryl having 1 to 3 heteroatoms independently selected from oxygen and nitrogen, wherein the pyridyl, the 9 to 10 membered fused heterobicyclic ring, and the 9 to 10 membered fused bicyclic heteroaryl represented by R³ are each optionally substituted with 1 to 2 substituents independently selected from C₁₋₄alkyl, halogen, halo-substitutedC₁₋₄ alkyl, hydroxyl and C₁₋₄alkoxy;
R^{8b} is H or halogen; and
R⁹ is C₁₋₅alkyl.

33. The compound of claim 32, or a pharmaceutically acceptable salt thereof, wherein:
R¹ is 6 or 7 membered bridged-heterocyclic ring having an oxygen atom, optionally substituted with a C₁₋₄alkyl;
R³ is pyridyl, pyridinyl-2(1H)-one, 9 membered fused heterobicyclic ring having an oxygen atom or a 9 membered fused bicyclic heteroaryl having 2 or 3 nitrogen atoms, wherein the pyridyl, the 9 membered fused heterobicyclic ring, and the 9 membered fused bicyclic heteroaryl represented by R³ are each optionally substituted with 1 to 2 substituents independently selected from halogen, halo-substitutedC₁₋₄ alkyl and C₁₋₄alkoxy;
R^{8b} is H or F; and
R⁹ is C₁₋₄alkyl.

34. The compound of claim 32, or a pharmaceutically acceptable salt thereof, wherein:
R¹ is selected from 2-oxabicyclo[2.1.1]hexan-4-yl 8-oxabicyclo[3.2.1]octan-3-yl, 2-oxabicyclo[2.2.1]heptan-4-yl, 2-oxabicyclo[2.2.2]octan-4-yl, 2-oxabicyclo[3.1.1]heptan-5-yl, each of which is optionally substituted with C₁₋₄alkyl;
R³ is selected from 2-pyridyl, 3-pyridyl, pyridinyl-2(1H)-one, 2,3-dihydrobenzofuran, pyrazolo[1,5-a]pyridine, and [1,2,4]triazolo[1,5-a]pyridine, each of which is optionally substituted with 1 to 2 substituents independently selected from halo, halo-substitutedC₁₋₄ alkyl and C₁₋₄alkoxy;
R⁹ is C₁₋₄alkyl.

35. The compound of claim 34, or a pharmaceutically acceptable salt thereof, wherein:
R¹ is selected from 2-oxabicyclo[2.1.1]hexan-4-yl 8-oxabicyclo[3.2.1]octan-3-yl, 2-oxabicyclo[2.2.1]heptan-4-yl, 2-oxabicyclo[2.2.2]octan-4-yl, 2-oxabicyclo[3.1.1]heptan-5-yl, each of which is optionally substituted with methyl;
R³ is selected from 2-pyridyl, 3-pyridyl, pyridinyl-2(1H)-one, 2,3-dihydrobenzofuran, pyrazolo[1,5-a]pyridine, and [1,2,4]triazolo[1,5-a]pyridine, each of which is optionally substituted with 1 to 2 substituents independently selected from —F, —CHF₂ and —OCH₃; and
R⁹ is —CH₃, —CH₂CH₃, or —C(CH₃)₂,

36. The compound of claim 1, selected from the group consisting of:
N-(7-ethoxy-2-ethylimidazo[1,2-a]pyridin-6-yl)-6-(trifluoromethyl)picolinamide;
N-(2-cyclopropyl-7-ethoxyimidazo[1,2-a]pyridin-6-yl)-6-(trifluoromethyl)picolinamide;
N-(2-cyclopropyl-7-ethoxyimidazo[1,2-a]pyridin-6-yl)-6-(difluoromethyl)picolinamide;
N-(2-cyclopropyl-7-methoxyimidazo[1,2-a]pyridin-6-yl)-6-(difluoromethyl)picolinamide;
N-(7-(cyclobutylmethoxy)-2-cyclopropylimidazo[1,2-a]pyridin-6-yl)-6-(difluoromethyl)picolinamide;
-3-yl)methoxy)imidazo[1,2-a]pyridin-6-yl)-6-(difluoromethyl)picolinamide;
N-(2-cyclopropyl-7-(2-(2-oxopyridin-1(2H)-yl)propoxy)imidazo[1,2-a]pyridin-6-yl)-6-(difluoromethyl)picolinamide;
(R)—N-(2-cyclopropyl-7-(2-(2-oxopyridin-1(2H)-yl)propoxy)imidazo[1,2-a]pyridin-6-yl)-6-(difluoromethyl)picolinamide;
6-(difluoromethyl)-N-(7-ethoxy-2-(1-methyl-2-oxopiperidin-4-yl)imidazo[1,2-a]pyridin-6-yl)picolinamide;
(S)—N-(2-cyclopropyl-7-(2-(2-oxopyridin-1(2H)-yl)propoxy)imidazo[1,2-a]pyridin-6-yl)-6-(difluoromethyl)picolinamide;
(S)—N-(2-cyclopropyl-7-(2-(5-oxopyrrolidin-2-yl)ethoxy)imidazo[1,2-a]pyridin-6-yl)-6-(difluoromethyl)picolinamide;
N-(7-(benzyloxy)-2-cyclopropylimidazo[1,2-a]pyridin-6-yl)-6-(difluoromethyl)picolinamide;
N-(2-cyclopropyl-7-hydroxyimidazo[1,2-a]pyridin-6-yl)-6-(difluoromethyl)picolinamide;
N-(2-cyclopropyl-7-(((2S,3S,4S)-3-ethyl-4-fluoro-5-oxopyrrolidin-2-yl)methoxy)imidazo[1,2-a]pyridin-6-yl)-6-(difluoromethyl)picolinamide;
6-(difluoromethyl)-N-(7-ethoxy-2-(3-methoxypropyl)imidazo[1,2-a]pyridin-6-yl)picolinamide;
6-(difluoromethyl)-N-(7-ethoxy-2-(2-methoxyethyl)imidazo[1,2-a]pyridin-6-yl)picolinamide;
6-(difluoromethyl)-N-(7-ethoxy-2-((tetrahydrofuran-3-yl)methyl)imidazo[1,2-a]pyridin-6-yl)picolinamide;
(S)-6-(difluoromethyl)-N-(7-ethoxy-2-((tetrahydrofuran-3-yl)methyl)imidazo[1,2-a]pyridin-6-yl)picolinamide;
(R)-6-(difluoromethyl)-N-(7-ethoxy-2-((tetrahydrofuran-3-yl)methyl)imidazo[1,2-a]pyridin-6-yl)picolinamide;
6-(difluoromethyl)-N-(7-ethoxy-2-(1-hydroxy-2-methylpropan-2-yl)imidazo[1,2-a]pyridin-6-yl)picolinamide;
N-(7-ethoxy-2-(hydroxymethyl)imidazo[1,2-a]pyridin-6-yl)-6-(trifluoromethyl)picolinamide;
6-(difluoromethyl)-N-(7-ethoxy-2-(5-oxaspiro[2.4]heptan-1-yl)imidazo[1,2-a]pyridin-6-yl)picolinamide;
N-(7-ethoxy-2-(2-hydroxypropan-2-yl)imidazo[1,2-a]pyridin-6-yl)-6-(trifluoromethyl)picolinamide;
N-(7-ethoxy-2-(fluoromethyl)imidazo[1,2-a]pyridin-6-yl)-6-(trifluoromethyl)picolinamide;
6-(difluoromethyl)-N-(7-ethoxy-2-(3-hydroxypropyl)imidazo[1,2-a]pyridin-6-yl)picolinamide;
N-(2-cyclopropyl-7-(4-(2,2-difluoroethyl)piperazin-1-yl)imidazo[1,2-a]pyridin-6-yl)-6-(trifluoromethyl)picolinamide;
N-(2-cyclopropyl-7-(cyclopropylmethoxy)imidazo[1,2-a]pyridin-6-yl)-6-(difluoromethyl)picolinamide;
N-(2-cyclopropyl-7-(2,2,2-trifluoroethoxy)imidazo[1,2-a]pyridin-6-yl)-6-(difluoromethyl)picolinamide;
6-(difluoromethyl)-N-(7-ethoxy-2-(3-hydroxy-3-methylbutyl)imidazo[1,2-a]pyridin-6-yl)picolinamide;
N-(2-cyclopropyl-8-ethoxyimidazo[1,2-a]pyridin-6-yl)-6-(difluoromethyl)picolinamide;
N-(2-cyclopropyl-7-ethoxy-3-fluoroimidazo[1,2-a]pyridin-6-yl)-6-(difluoromethyl)picolinamide;
N-(2-cyclopropylimidazo[1,2-a]pyridin-6-yl)-6-(difluoromethyl)picolinamide;
N-(2-cyclopropylimidazo[1,2-a]pyrazin-6-yl)-6-(difluoromethyl)picolinamide;
6-acetamido-N-(7-ethoxy-2-ethylimidazo[1,2-a]pyridin-6-yl)picolinamide;
N-(2-(3,3-difluorocyclobutyl)-7-ethoxyimidazo[1,2-a]pyridin-6-yl)-6-(trifluoromethyl)picolinamide;
N-(7-ethoxy-2-ethylimidazo[1,2-a]pyridin-6-yl)-5,6-dimethylpicolinamide;
N-(7-ethoxy-2-ethylimidazo[1,2-a]pyridin-6-yl)-3,4-difluorobenzamide;
N-(7-ethoxy-2-ethylimidazo[1,2-a]pyridin-6-yl)-3-fluoro-6-(trifluoromethyl)picolinamide;
N-(7-ethoxy-2-ethylimidazo[1,2-a]pyridin-6-yl)-1,3-dihydroisobenzofuran-5-carboxamide;
N-(7-ethoxy-2-ethylimidazo[1,2-a]pyridin-6-yl)-5,6-dihydro-4H-pyrrolo[1,2-b]pyrazole-2-carboxamide;
N-(7-ethoxy-2-ethylimidazo[1,2-a]pyridin-6-yl)-4-methyl-3,4-dihydro-2H-benzo[b][1,4]oxazine-8-carboxamide;
N-(7-ethoxy-2-ethylimidazo[1,2-a]pyridin-6-yl)-2,4-difluorobenzamide;
N-(7-ethoxy-2-ethylimidazo[1,2-a]pyridin-6-yl)-2-oxoindoline-6-carboxamide;
N-(7-ethoxy-2-ethylimidazo[1,2-a]pyridin-6-yl)-2,3-dihydrobenzofuran-6-carboxamide;
N-(7-ethoxy-2-ethylimidazo[1,2-a]pyridin-6-yl)isochromane-7-carboxamide;
1-(difluoromethyl)-N-(7-ethoxy-2-ethylimidazo[1,2-a]pyridin-6-yl)-1H-pyrazole-3-carboxamide;
N-(7-ethoxy-2-ethylimidazo[1,2-a]pyridin-6-yl)-4,5-dimethylpicolinamide;
5-chloro-N-(7-ethoxy-2-ethylimidazo[1,2-a]pyridin-6-yl)picolinamide;
N-(7-ethoxy-2-ethylimidazo[1,2-a]pyridin-6-yl)isochromane-6-carboxamide;
N-(7-ethoxy-2-ethylimidazo[1,2-a]pyridin-6-yl)-4,5-dimethylthiazole-2-carboxamide;
N-(7-ethoxy-2-ethylimidazo[1,2-a]pyridin-6-yl)-6,7-dihydro-5H-cyclopenta[b]pyridine-2-carboxamide;
N-(7-ethoxy-2-ethylimidazo[1,2-a]pyridin-6-yl)-5-fluoro-6-(trifluoromethyl)picolinamide;
N-(2-cyclopropyl-7-methoxyimidazo[1,2-a]pyridin-6-yl)-1-methyl-2-oxo-1,2-dihydropyridine-3-carboxamide;
N-(2-cyclopropyl-7-ethoxyimidazo[1,2-a]pyridin-6-yl)pyrazolo[1,5-a]pyrimidine-2-carboxamide;
N-(2-cyclopropyl-7-ethoxyimidazo[1,2-a]pyridin-6-yl)-3-(trifluoromethyl)benzamide;
N-(7-ethoxy-2-(ethoxymethyl)imidazo[1,2-a]pyridin-6-yl)-6-(trifluoromethyl)picolinamide;
N-(7-ethoxy-2-(methoxymethyl)imidazo[1,2-a]pyridin-6-yl)-6-(trifluoromethyl)picolinamide;
N-(2-(difluoromethyl)-7-ethoxyimidazo[1,2-a]pyridin-6-yl)-6-(trifluoromethyl)picolinamide;
N-(7-ethoxy-2,3-dimethylimidazo[1,2-a]pyridin-6-yl)-6-(trifluoromethyl)picolinamide;
N-(7-ethoxy-2-(tetrahydrofuran-3-yl)imidazo[1,2-a]pyridin-6-yl)-6-(trifluoromethyl)picolinamide;
N-(7-ethoxy-2-((tetrahydrofuran-2-yl)methyl)imidazo[1,2-a]pyridin-6-yl)-6-(trifluoromethyl)picolinamide;
N-(2-(azetidin-3-yl)-7-ethoxyimidazo[1,2-a]pyridin-6-yl)-6-(trifluoromethyl)picolinamide;
N-(7-ethoxy-2-(pyrrolidin-3-yl)imidazo[1,2-a]pyridin-6-yl)-6-(trifluoromethyl)picolinamide;
N-(7-ethoxy-2-(piperidin-4-yl)imidazo[1,2-a]pyridin-6-yl)-6-(trifluoromethyl)picolinamide;
N-(7-ethoxy-1,2,3,4-tetrahydroimidazo[1,2-a:5,4-c′]dipyridin-8-yl)-6-(trifluoromethyl)picolinamide;
N-(7-ethoxy-2-(1-methylazetidin-3-yl)imidazo[1,2-a]pyridin-6-yl)-6-(trifluoromethyl)picolinamide;
N-(7-ethoxy-2-(1-methylpyrrolidin-3-yl)imidazo[1,2-a]pyridin-6-yl)-6-(trifluoromethyl)picolinamide;
N-(7-ethoxy-2-(1-methylpiperidin-4-yl)imidazo[1,2-a]pyridin-6-yl)-6-(trifluoromethyl)picolinamide;
N-(2-(1-acetylazetidin-3-yl)-7-ethoxyimidazo[1,2-a]pyridin-6-yl)-6-(trifluoromethyl)picolinamide;
N-(2-(1-acetylpyrrolidin-3-yl)-7-ethoxyimidazo[1,2-a]pyridin-6-yl)-6-(trifluoromethyl)picolinamide;
N-(2-(1-acetylpiperidin-4-yl)-7-ethoxyimidazo[1,2-a]pyridin-6-yl)-6-(trifluoromethyl)picolinamide;
N-(2-cyclopropyl-7-ethoxyimidazo[1,2-a]pyridin-6-yl)-2-(2,2,2-trifluoroethoxy)nicotinamide;
N-(2-cyclopropyl-7-ethoxyimidazo[1,2-a]pyridin-6-yl)-2-oxo-1-(2,2,2-trifluoroethyl)-1,2-dihydropyridine-3-carboxamide;
6-(difluoromethyl)-N-(7-ethoxy-2-ethylimidazo[1,2-a]pyrimidin-6-yl)picolinamide;
N-(7-ethoxy-2-((tetrahydrofuran-3-yl)methyl)imidazo[1,2-a]pyrimidin-6-yl)-6-(trifluoromethyl)picolinamide;
6-(difluoromethyl)-N-(2-ethyl-8-fluoroimidazo[1,2-a]pyridin-6-yl)picolinamide;
6-(difluoromethyl)-N-(7-ethoxy-2-(1-(2,2,2-trifluoroethyl)azetidin-3-yl)imidazo[1,2-a]pyridin-6-yl)picolinamide;
N-(2-(1-(2,2-difluoroethyl)azetidin-3-yl)-7-ethoxyimidazo[1,2-a]pyridin-6-yl)-6-(difluoromethyl)picolinamide;
6-(difluoromethyl)-N-(7-ethoxy-2-(1-(oxetan-3-yl)azetidin-3-yl)imidazo[1,2-a]pyridin-6-yl)picolinamide;
6-(difluoromethyl)-N-(7-ethoxy-2-(1-(2-methoxyethyl)azetidin-3-yl)imidazo[1,2-a]pyridin-6-yl)picolinamide;
N-(2-(1-acetylazetidin-3-yl)-7-ethoxyimidazo[1,2-a]pyridin-6-yl)-6-(difluoromethyl)picolinamide;
N-(2-((1H-1,2,4-triazol-1-yl)methyl)-7-ethoxyimidazo[1,2-a]pyridin-6-yl)-6-(difluoromethyl)picolinamide;
N-(2-((1,4-dioxan-2-yl)methyl)-7-ethoxyimidazo[1,2-a]pyridin-6-yl)-6-(difluoromethyl)picolinamide;
6-(difluoromethyl)-N-(7-ethoxy-2-((1-methyl-1H-pyrazol-3-yl)methyl)imidazo[1,2-a]pyridin-6-yl)picolinamide;
6-(difluoromethyl)-N-(7-ethoxy-2-((2-oxopyrrolidin-1-yl)methyl)imidazo[1,2-a]pyridin-6-yl)picolinamide;
6-(difluoromethyl)-N-(7-ethoxy-2-((pyridin-2-yloxy)methyl)imidazo[1,2-a]pyridin-6-yl)picolinamide;
6-(difluoromethyl)-N-(7-ethoxy-2-((2-oxopyridin-1(2H)-yl)methyl)imidazo[1,2-a]pyridin-6-yl)picolinamide;
6-(difluoromethyl)-N-(7-ethoxy-2-(4-hydroxytetrahydro-2H-pyran-4-yl)imidazo[1,2-a]pyridin-6-yl)picolinamide;
6-(difluoromethyl)-N-(7-ethoxy-2-(4-ethoxytetrahydro-2H-pyran-4-yl)imidazo[1,2-a]pyridin-6-yl)picolinamide;
1-(difluoromethyl)-N-(7-ethoxy-2-(3-methoxypropyl)imidazo[1,2-a]pyridin-6-yl)-1H-pyrazole-3-carboxamide;
N-(2-((1R,6R,7R)-3-oxabicyclo[4.1.0]heptan-7-yl)-7-ethoxyimidazo[1,2-a]pyridin-6-yl)-6-(difluoromethyl)picolinamide;
N-(2-cyclopropylimidazo[1,2-a]pyrimidin-6-yl)-6-(trifluoromethyl)picolinamide;
(S)—N-(7-ethoxy-2-((tetrahydrofuran-3-yl)methyl)imidazo[1,2-a]pyrimidin-6-yl)-6-(trifluoromethyl)picolinamide;
(R)—N-(7-ethoxy-2-((tetrahydrofuran-3-yl)methyl)imidazo[1,2-a]pyrimidin-6-yl)-6-(trifluoromethyl)picolinamide;
N-(7-ethoxy-2-((1r,3r)-3-hydroxycyclobutyl)imidazo[1,2-a]pyridin-6-yl)-6-(trifluoromethyl)picolinamide;
N-(7-ethoxy-2-((1s,3s)-3-hydroxycyclobutyl)imidazo[1,2-a]pyridin-6-yl)-6-(trifluoromethyl)picolinamide;
6-(difluoromethyl)-N-(7-ethoxy-2-(1-(hydroxymethyl)cyclopropyl)imidazo[1,2-a]pyridin-6-yl)picolinamide;
6-(difluoromethyl)-N-(7-ethoxy-2-(2-fluoro-3-hydroxypropyl)imidazo[1,2-a]pyridin-6-yl)picolinamide;
6-(difluoromethyl)-N-(7-ethoxy-2-(2-hydroxypropan-2-yl)imidazo[1,2-a]pyridin-6-yl)picolinamide;
N-(2-cyclopropyl-7-(2-methoxyethoxy)imidazo[1,2-a]pyridin-6-yl)-6-(difluoromethyl)picolinamide;
N-(2-cyclopropyl-7-(2-morpholinoethoxy)imidazo[1,2-a]pyridin-6-yl)-6-(difluoromethyl)picolinamide;
N-(8-(benzyloxy)-2-(tetrahydro-2H-pyran-4-yl)imidazo[1,2-a]pyridin-6-yl)-6-(trifluoromethyl)picolinamide;
N-(8-hydroxy-2-(tetrahydro-2H-pyran-4-yl)imidazo[1,2-a]pyridin-6-yl)-6-(trifluoromethyl)picolinamide;
N-(8-methoxy-2-tetrahydropyran-4-yl-imidazo[1,2-a]pyridin-6-yl)-6-(trifluoromethyl)picolinamide;
N-(8-bromo-2-(tetrahydro-2H-pyran-4-yl)imidazo[1,2-a]pyridin-6-yl)-6-(trifluoromethyl)picolinamide;
N-(8-(3,6-dihydro-2H-pyran-4-yl)-2-(tetrahydro-2H-pyran-4-yl)imidazo[1,2-a]pyridin-6-yl)-6-(trifluoromethyl)picolinamide;
6-(6-(difluoromethyl)picolinamido)-7-ethoxy-N,N-dimethylimidazo[1,2-a]pyridine-2-carboxamide;
N-(2-(1,1-difluoroethyl)-7-ethoxyimidazo[1,2-a]pyridin-6-yl)-6-(difluoromethyl)picolinamide;
6-(difluoromethyl)-N-(2-(3,3-difluoropropyl)-7-ethoxyimidazo[1,2-a]pyridin-6-yl)picolinamide;
6-(difluoromethyl)-N-(7-ethoxy-2-(1-fluorocyclopropyl)imidazo[1,2-a]pyridin-6-yl)picolinamide;
N-(7-ethoxy-2-(tetrahydro-2H-pyran-3-yl)imidazo[1,2-a]pyridin-6-yl)-6-(trifluoromethyl)picolinamide;
N-(2-(2,2-dimethyltetrahydro-2H-pyran-4-yl)-7-ethoxyimidazo[1,2-a]pyridin-6-yl)-6-(trifluoromethyl)picolinamide;
6-(difluoromethyl)-N-(7-ethoxy-2-((1r,3r)-3-methoxycyclobutyl)imidazo[1,2-a]pyridin-6-yl)picolinamide;
6-(difluoromethyl)-N-(7-ethoxy-2-((1s,3s)-3-methoxycyclobutyl)imidazo[1,2-a]pyridin-6-yl)picolinamide;
6-(difluoromethyl)-N-(7-ethoxy-2-(tetrahydro-2H-pyran-4-yl)imidazo[1,2-a]pyridin-6-yl)picolinamide;
N-(2-cyclopropyl-7-ethoxyimidazo[1,2-a]pyridin-6-yl)-6-methoxypicolinamide;
N-(2-cyclopropyl-7-ethoxyimidazo[1,2-a]pyridin-6-yl)-6-(trifluoromethoxy)picolinamide;
N-(2-cyclopropyl-7-ethoxyimidazo[1,2-a]pyridin-6-yl)-2,3-dihydrobenzofuran-7-carboxamide;
N-(2-cyclopropyl-7-ethoxyimidazo[1,2-a]pyridin-6-yl)-6-(trifluoromethyl)pyrazine-2-carboxamide;
6-(difluoromethyl)-N-(7-ethoxy-2-(morpholinomethyl)imidazo[1,2-a]pyridin-6-yl)picolinamide;
N-(7-ethoxy-2-((tetrahydrofuran-3-yl)methyl)imidazo[1,2-a]pyridin-6-yl)-1-isopropyl-1H-pyrazole-3-carboxamide;
N-(7-ethoxy-2-((tetrahydrofuran-3-yl)methyl)imidazo[1,2-a]pyridin-6-yl)-1-(2,2,2-trifluoroethyl)-1H-pyrazole-3-carboxamide;
1-(difluoromethyl)-N-(7-ethoxy-2-((tetrahydrofuran-3-yl)methyl)imidazo[1,2-a]pyridin-6-yl)-1H-pyrazole-3-carboxamide;
1-(2,2-difluoroethyl)-N-(7-ethoxy-2-((tetrahydrofuran-3-yl)methyl)imidazo[1,2-a]pyridin-6-yl)-1H-pyrazole-3-carboxamide;
6-(difluoromethyl)-N-(7-ethoxy-2-(morpholine-4-carbonyl)imidazo[1,2-a]pyridin-6-yl)picolinamide;
6-(difluoromethyl)-N-(7-ethoxy-2-(3-fluorobicyclo[1.1.1]pentan-1-yl)imidazo[1,2-a]pyridin-6-yl)picolinamide;
6-(difluoromethyl)-N-(7-ethoxy-2-(1-methoxycyclopropyl)imidazo[1,2-a]pyridin-6-yl)picolinamide;
6-(difluoromethyl)-N-(7-ethoxy-2-(3-(2-hydroxypropan-2-yl)bicyclo[1.1.1]pentan-1-yl)imidazo[1,2-a]pyridin-6-yl)picolinamide;
6-(difluoromethyl)-N-(7-ethoxy-2-(3-(hydroxymethyl)bicyclo[1.1.1]pentan-1-yl)imidazo[1,2-a]pyridin-6-yl)picolinamide;
N-(2-cyclopropyl-7-ethoxyimidazo[1,2-a]pyridin-6-yl)-6-morpholinopicolinamide;
N-(2-cyclopropyl-7-ethoxyimidazo[1,2-a]pyridin-6-yl)-6-(1-hydroxycyclobutyl)picolinamide;
N-(7-ethoxy-2-(tetrahydro-2H-pyran-4-yl)imidazo[1,2-a]pyridin-6-yl)-6-methoxypicolinamide;
N-(7-ethoxy-2-((tetrahydrofuran-3-yl)methyl)imidazo[1,2-a]pyridin-6-yl)-6-methoxypicolinamide;
6-(difluoromethyl)-N-(7-ethoxy-2-((1r,3r)-3-isopropoxycyclobutyl)imidazo[1,2-a]pyridin-6-yl)picolinamide;
6-(difluoromethyl)-N-(7-ethoxy-2-((1s,3s)-3-isopropoxycyclobutyl)imidazo[1,2-a]pyridin-6-yl)picolinamide;
N-(7-ethoxy-2-(1-(tetrahydrofuran-3-yl)ethyl)imidazo[1,2-a]pyridin-6-yl)-6-(trifluoromethyl)picolinamide;
N-(7-ethoxy-2-((R)-1-((S)-tetrahydrofuran-3-yl)ethyl)imidazo[1,2-a]pyridin-6-yl)-6-(trifluoromethyl)picolinamide;
N-(7-ethoxy-2-((S)-1-((R)-tetrahydrofuran-3-yl)ethyl)imidazo[1,2-a]pyridin-6-yl)-6-(trifluoromethyl)picolinamide;
N-(7-ethoxy-2-((S)-1-((S)-tetrahydrofuran-3-yl)ethyl)imidazo[1,2-a]pyridin-6-yl)-6-(trifluoromethyl)picolinamide;
N-(7-ethoxy-2-((R)-1-((R)-tetrahydrofuran-3-yl)ethyl)imidazo[1,2-a]pyridin-6-yl)-6-(trifluoromethyl)picolinamide;
N-(7-ethoxy-2-(fluoro(tetrahydrofuran-3-yl)methyl)imidazo[1,2-a]pyridin-6-yl)-6-(trifluoromethyl)picolinamide;
N-(7-ethoxy-2-((R)-fluoro((R)-tetrahydrofuran-3-yl)methyl)imidazo[1,2-a]pyridin-6-yl)-6-(trifluoromethyl)picolinamide;
N-(7-ethoxy-2-((S)-fluoro((S)-tetrahydrofuran-3-yl)methyl)imidazo[1,2-a]pyridin-6-yl)-6-(trifluoromethyl)picolinamide;
N-(7-ethoxy-2-((S)-fluoro((R)-tetrahydrofuran-3-yl)methyl)imidazo[1,2-a]pyridin-6-yl)-6-(trifluoromethyl)picolinamide;
N-(7-ethoxy-2-((R)-fluoro((S)-tetrahydrofuran-3-yl)methyl)imidazo[1,2-a]pyridin-6-yl)-6-(trifluoromethyl)picolinamide;
6-(difluoromethyl)-N-(7-ethoxy-2-(1-methyl-2-oxopyrrolidin-3-yl)imidazo[1,2-a]pyridin-6-yl)picolinamide;
N-(8-ethoxy-2-(tetrahydro-2H-pyran-4-yl)imidazo[1,2-a]pyrazin-6-yl)-6-methoxypicolinamide;
6-(difluoromethyl)-N-(2-ethyl-[1,2,4]triazolo[1,5-a]pyridin-6-yl)picolinamide;
N-(2-(1,1-difluoro-3-methoxypropyl)-7-ethoxyimidazo[1,2-a]pyridin-6-yl)-6-(difluoromethyl)picolinamide;
6-(difluoromethyl)-N-(7-ethoxy-2-(tetrahydrothiophen-3-yl)imidazo[1,2-a]pyridin-6-yl)picolinamide;
6-(difluoromethyl)-N-(7-ethoxy-2-((tetrahydro-2H-pyran-4-yl)methyl)imidazo[1,2-a]pyridin-6-yl)picolinamide;
N-(2-(2-cyanopropan-2-yl)-7-ethoxyimidazo[1,2-a]pyridin-6-yl)-6-(difluoromethyl)picolinamide;
6-(difluoromethyl)-N-(7-ethoxy-2-(3-methoxybicyclo[1.1.1]pentan-1-yl)imidazo[1,2-a]pyridin-6-yl)picolinamide;
N-(7-ethoxy-2-((tetrahydrofuran-3-yl)methyl)imidazo[1,2-a]pyridin-6-yl)-4-methyloxazole-2-carboxamide;
N-(7-ethoxy-2-((tetrahydrofuran-3-yl)methyl)imidazo[1,2-a]pyridin-6-yl)-1-methyl-1H-pyrazole-3-carboxamide;
N-(7-ethoxy-2-((tetrahydrofuran-3-yl)methyl)imidazo[1,2-a]pyridin-6-yl)-2-(1-methyl-1H-pyrazol-4-yl)oxazole-4-carboxamide;
N-(2-((1R,5S,6s)-3-oxabicyclo[3.1.0]hexan-6-yl)-7-ethoxyimidazo[1,2-a]pyridin-6-yl)-6-(difluoromethyl)picolinamide;
N-(7-ethoxy-2-((tetrahydrofuran-3-yl)methyl)imidazo[1,2-a]pyridin-6-yl)-1-methyl-1H-1,2,4-triazole-3-carboxamide;
N-(7-ethoxy-2-((tetrahydrofuran-3-yl)methyl)imidazo[1,2-a]pyridin-6-yl)-1-methyl-1H-imidazole-4-carboxamide;
N-(7-ethoxy-2-((tetrahydrofuran-3-yl)methyl)imidazo[1,2-a]pyridin-6-yl)-3-methyl-1,2,4-oxadiazole-5-carboxamide;
N-(7-ethoxy-2-((tetrahydrofuran-3-yl)methyl)imidazo[1,2-a]pyridin-6-yl)-2-methyloxazole-4-carboxamide;
N-(2-(4-cyanotetrahydro-2H-pyran-4-yl)-7-ethoxyimidazo[1,2-a]pyridin-6-yl)-6-(difluoromethyl)picolinamide;
1-(difluoromethyl)-N-(7-((3-methyloxetan-3-yl)methoxy)-2-(tetrahydro-2H-pyran-4-yl)imidazo[1,2-a]pyridin-6-yl)-1H-pyrazole-4-carboxamide;
1-(difluoromethyl)-N-(2-(tetrahydro-2H-pyran-4-yl)imidazo[1,2-a]pyrazin-6-yl)-1H-pyrazole-4-carboxamide;
N-(7-((3-methyloxetan-3-yl)methoxy)-2-((tetrahydrofuran-3-yl)methyl)imidazo[1,2-a]pyridin-6-yl)-6-(trifluoromethyl)picolinamide;
(R)—N-(7-((3-methyloxetan-3-yl)methoxy)-2-((tetrahydrofuran-3-yl)methyl)imidazo[1,2-a]pyridin-6-yl)-6-(trifluoromethyl)picolinamide;
(S)—N-(7-((3-methyloxetan-3-yl)methoxy)-2-((tetrahydrofuran-3-yl)methyl)imidazo[1,2-a]pyridin-6-yl)-6-(trifluoromethyl)picolinamide;
N-(7-((3-methyloxetan-3-yl)methoxy)-2-(tetrahydro-2H-pyran-4-yl)imidazo[1,2-a]pyridin-6-yl)-6-(trifluoromethyl)picolinamide;
6-(difluoromethyl)-N-(7-isopropoxy-2-(tetrahydro-2H-pyran-4-yl)imidazo[1,2-a]pyridin-6-yl)picolinamide;
6-(difluoromethyl)-N-(7-(2-(dimethylamino)ethoxy)-2-(tetrahydro-2H-pyran-4-yl)imidazo[1,2-a]pyridin-6-yl)picolinamide;
6-(difluoromethyl)-N-(2-(2-hydroxypropan-2-yl)pyrazolo[1,5-a]pyridin-5-yl)picolinamide;
5-(6-(difluoromethyl)picolinamido)-N,N-dimethylpyrazolo[1,5-a]pyridine-2-carboxamide;
N-(2-(morpholine-4-carbonyl)pyrazolo[1,5-a]pyridin-5-yl)-6-(trifluoromethyl)picolinamide;
N-(2-(1-morpholinoethyl)pyrazolo[1,5-a]pyridin-5-yl)-6-(trifluoromethyl)picolinamide;
N-(2-cyclopropyl-6-methoxypyrazolo[1,5-a]pyridin-5-yl)-6-(difluoromethyl)picolinamide;
N-(2-cyclopropyl-7-((3-methyloxetan-3-yl)methoxy)imidazo[1,2-a]pyridin-6-yl)-6-(difluoromethyl)picolinamide;
N-(2-(cyclopropyl(hydroxy)methyl)-7-ethoxyimidazo[1,2-a]pyridin-6-yl)-6-(trifluoromethyl)picolinamide;
(S)—N-(2-(cyclopropyl(hydroxy)methyl)-7-ethoxyimidazo[1,2-a]pyridin-6-yl)-6-(trifluoromethyl)picolinamide; and
(R)—N-(2-(cyclopropyl(hydroxy)methyl)-7-ethoxyimidazo[1,2-a]pyridin-6-yl)-6-(trifluoromethyl)picolinamide;
or a pharmaceutically acceptable salt thereof.

37. A pharmaceutical composition comprising a compound of any one of the preceding claims or a pharmaceutically acceptable salt thereof.

38. The pharmaceutical composition of claim 37, further comprising one or more additional pharmaceutical agent(s).

39. A method of treating an iRak4 mediated disease in a subject comprising administering to the subject a compound or a pharmaceutically acceptable salt thereof of any one of claims 1 to 16 or a pharmaceutical composition of any one of claims 37 to 38.

40. The method of claim 39, wherein the iRak4 mediated disease is selected from the group consisting from Rheumatoid Arthritis, Psoriatic arthritis, Osteoarthritis, Systemic Lupus Erythematosus, Lupus nephritis, Ankylosing Spondylitis, Osteoporosis, Systemic sclerosis, Multiple Sclerosis, Psoriasis, Type I diabetes, Type II diabetes, Inflammatory Bowel Disease, Cronh's Disease, Ulcerative Colitis, Hyperimmunoglobulinemia D, periodic fever syndrome, Cryopyrin-associated periodic syndromes, Schnitzler's syndrome, Systemic juvenile idiopathic arthritis, Adult's onset Still's disease, Gout, Pseudogout, SAPHO syndrome, Castleman's disease, Sepsis, Stroke, Atherosclerosis, Celiac disease, Deficiency of IL-1 Receptor Antagonist, Alzheimer's disease, Parkinson's disease, and Cancer.

41. The method of claim 39, wherein the iRak mediated disease is selected from the group consisting from is selected from an autoimmune disease, an inflammatory disease, bone diseases, metabolic diseases, neurological and neurodegenerative diseases and/or disorders, cardiovascular diseases, allergies, asthma, hormone-related diseases, Ischemic stroke, Cerebral Ischemia, hypoxia, Traumatic Brain Injury, Chronic Traumatic Encephalopathy, epilepsy, multiple sclerosis, Parkinson's disease, and Amyotrophic Lateral Sclerosis.
